(19) United States
(12) Patent Application Publication
(10) Pub. No.: US 2024/0182510 A1
(43) Pub. Date: Jun. 6, 2024

(54) Compounds Useful in HIV Treatment

(71) Applicant: ViiV Healthcare Company, Wilmington, DE (US)

(72) Inventor: B. Narasimhulu NAIDU, Branford, CT (US)

(73) Assignee: ViiV Healthcare Company, Wilmington, DE (US)

(21) Appl. No.: 18/547,001

(22) PCT Filed: Feb. 21, 2022
(86) PCT No.: PCT/US2022/017134

§ 371 Date: Aug. 18, 2023

Related U.S. Application Data
(60) Provisional application No. 63/152,356, filed on Feb. 23, 2021.

(51) Int. Cl. C07H 19/20

(52) U.S. Cl. CPC C07H 19/20

(57) ABSTRACT

The invention relates to compounds of Formula (I), salts thereof, pharmaceutical compositions thereof, as well as methods of treating or preventing HIV in subjects.

### FIELD OF THE INVENTION

The present invention relates to compounds, pharmaceutical compositions, and methods of use thereof. In particular, such methods of use encompass e.g., methods for treating HIV and methods of preventing HIV.

### BACKGROUND OF THE INVENTION

Human immunodeficiency virus type 1 (HIV-1) infection leads to the contraction of acquired immune deficiency disease (AIDS). The number of cases of HIV continues to rise, and currently an estimated over thirty-five million individuals worldwide suffer from HIV infection e.g., http://www.sciencedirect.com/science/article/pii/S235230181630087X? via %3Dihub

Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still believed to be required due to a number of issues including, but not limited to undesirable drug-drug interactions; drug-food interactions; non-adherence to therapy; drug resistance due to mutation of the enzyme target; and inflammation related to the immunologic damage caused by the HIV infection.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur and the survival and quality of life are not normalized as compared to uninfected persons [Lohse Ann Intern Med 2007 146; 87-95]. Indeed, the incidence of several non-AIDS morbidities and mortalities, such as cardiovascular disease, frailty, and neurocognitive impairment, are increased in HAART-suppressed, HIV-infected subjects [Deeks Annu Rev Med 2011; 62:141-155]. This increased incidence of non-AIDS morbidity/mortality occurs in the context of, and is potentially caused by, elevated systemic inflammation related to the immunologic damage caused by HIV infection [Hunt J Infect Dis 2014][Byakagwa J Infect Dis 2014][Tenorio J Infect Dis 2014].

Modern antiretroviral therapy (ART) has the ability to effectively suppress HIV replication and improve health outcomes for HIV-infected persons, but is believed to not be capable of completely eliminating HIV viral reservoirs within the individual. HIV genomes can remain latent within mostly immune cells in the infected individual and may reactivate at any time, such that after interruption of ART, virus replication typically resumes within weeks. In a handful of individuals, the size of this viral reservoir has been significantly reduced and upon cessation of ART, the rebound of viral replication has been delayed [Henrich T J J Infect Dis 2013][Henrich T J Ann Intern Med 2014]. In one case, the viral reservoir was eliminated during treatment of leukemia and no viral rebound was observed during several years of follow-up [Hutter G N Engl J Med 2009]. These examples suggest the concept that reduction or elimination of the viral reservoir may be possible and can lead to viral remission or cure. As such, ways have been pursued to eliminate the viral reservoir, by direct molecular means, including excision of viral genomes with CRISPR/Cas9 systems, or to induce reactivation of the latent reservoir during ART so that the latent cells are eliminated. Induction of the latent reservoir typically results in either direct death of the latently infected cell or killing of the induced cell by the immune system after the virus is made visible. As this is performed during ART, viral genomes produced are believed to not result in the infection of new cells and the size of the reservoir may decay.

HAART therapies are often complex because a combination of different drugs must be administered often daily to the patient to avoid the rapid emergence of drug-resistant HIV-1 variants. Despite the positive impact of HAART on patient survival, drug resistance can still occur.

Current guidelines recommend that therapy includes three fully active drugs. See e.g. https://aidsinfo.nih.gov/guidelines. Additionally, two drug combinations may be employed as therapeutic regimens. Typically, first-line therapies combine two to three drugs targeting the viral enzymes reverse transcriptase and integrase. It is believed that sustained successful treatment of HIV-1-infected patients with antiretroviral drugs employ the continued development of new and improved drugs that are effective against HIV strains that have formed resistance to approved drugs. For example, an individual on a regimen containing 3TC/FTC (lamivudine/emtricitabine) may select for the M184V mutation that reduces susceptibility to these drugs by >100 fold. See e g., https://hivdb.stanford.edu/dr-summary/resistance-notes/NRTI

Another way to potentially address preventing formation of mutations is to increase patient adherence to a drug regimen. One manner that may be employed to accomplish this is by reducing the dosing frequency. For parenteral administration, it is believed to be advantageous to have drug substances with high lipophilicity in order to reduce solubility and limit the release rate within interstitial fluid. However, most nucleoside reverse transcriptase inhibitors are hydrophilic thereby potentially limiting their use as long acting parenteral agents.

There remains a need for compounds which may address the shortcomings set forth above.

### SUMMARY OF THE INVENTION

In one aspect, the invention provides a compound of the formula (I):

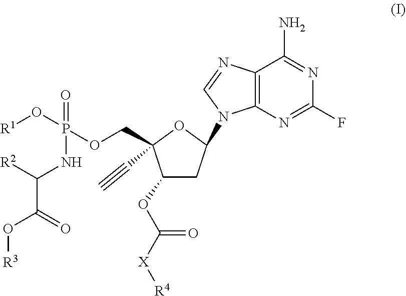

wherein
R¹ is selected from the group consisting of (C₁-C₂₅) alkyl, (C₁-C₂₅) alkylene-aryl, (C₁-C₂₀) alkylene-CO₂R⁵ and aryl;
R² is selected from the group consisting of (C₁-C₁₀) alkyl; (C₁-C₁₀) alkylene-aryl and aryl;
R³ is selected from the group consisting of (C₁-C₂₅) alkyl, (C₁-C₂₅) alkylene-aryl and aryl; and
R⁴ is selected from the group consisting of (C₁-C₂₅) alkyl, (C₁-C₂₅) alkylene-aryl, (C₁-C₂₀) alkylene-CO₂R⁵ and aryl; X is a bond, NR⁶, or O
R⁵ is selected from the group consisting of (C₁-C₂₅) alkyl, (C₁-C₂₅) alkylene-aryl and aryl;
R⁶ is H, (C₁-C₂₅) alkyl, (C₁-C₂₅) alkylene-aryl, (C₁-C₂₀) alkylene-CO₂R⁵ and aryl; and
wherein each of R¹, R², R³, R⁴, R⁵ and R⁶ may be independently and optionally substituted by one or more (C₁-C₁₄) alkyl, Cl, F, oxo, or (C₁-C₆) alkoxy;
or a pharmaceutically acceptable salt thereof.

In another aspect, the invention provides pharmaceutical compositions comprising a compound of Formula (I) or a pharmaceutically acceptable salt thereof and an excipient.

In another aspect, the invention provides a combination comprising a compound of Formula (I) or pharmaceutically acceptable salt thereof and one or more pharmaceutical agents active against HIV.

In another aspect, the invention provides a method of treating an HIV infection in a patient comprising administering to the patient a compound of Formula (I), or a pharmaceutically acceptable salt thereof.

In another aspect, the invention provides a method of preventing an HIV infection in a subject at risk for developing an HIV infection, comprising administering to the subject a compound of Formula (I), or a pharmaceutically acceptable salt thereof.

In another aspect, there is provided a compound of Formula (I) or a pharmaceutically acceptable salt thereof for use in therapy.

In another aspect, there is provided a compound of Formula (I) or a pharmaceutically acceptable salt thereof for use in treating an HIV infection.

In another aspect, there is provided a compound of Formula (I) or a pharmaceutically acceptable salt thereof for use in preventing an HIV infection.

In another aspect, there is provided the use of a compound of Formula (I) or a pharmaceutically acceptable salt thereof in the manufacture of a medicament for treating an HIV infection.

In another aspect, there is provided the use of a compound of Formula (I) or a pharmaceutically acceptable salt thereof in the manufacture of a medicament for preventing an HIV infection.

In another aspect, the invention provides a method of treating an HIV infection in a patient comprising administering to the patient a combination comprising a compound of Formula (I) or pharmaceutically acceptable salt thereof and one or more pharmaceutical agents active against HIV.

In another aspect, the invention provides a method of preventing an HIV infection in a subject at risk for developing an HIV infection, comprising administering to the subject a combination comprising a compound of Formula (I) or pharmaceutically acceptable salt thereof and one or more pharmaceutical agents active against HIV.

In another aspect, there is provided a combination comprising a compound of Formula (I) or pharmaceutically acceptable salt thereof and one or more pharmaceutical agents active against HIV for use in therapy.

In another aspect, there is provided a combination comprising a compound of Formula (I) or pharmaceutically acceptable salt thereof and one or more pharmaceutical agents active against HIV for use in treating an HIV infection.

In another aspect, there is provided a combination comprising a compound of Formula (I) or pharmaceutically acceptable salt thereof and one or more pharmaceutical agents active against HIV for use in preventing an HIV infection.

In another aspect, there is provided the use of a combination comprising a compound of Formula (I) or pharmaceutically acceptable salt thereof and one or more pharmaceutical agents active against HIV in the manufacture of a medicament for treating an HIV infection.

In another aspect, there is provided the use of a combination comprising a compound of Formula (I) or pharmaceutically acceptable salt thereof and one or more pharmaceutical agents active against HIV in the manufacture of a medicament for preventing an HIV infection.

These and other aspects are encompassed by the invention as set forth herein.

### DETAILED DESCRIPTION OF REPRESENTATIVE EMBODIMENTS

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings, unless otherwise noted herein by embodiments set forth.

The term “alkyl” represents a saturated, straight or branched hydrocarbon group. For the purposes of illustration, and as an example, the term “(C₁-C₂₅)alkyl” represents an alkyl group containing from 1 to 6 carbon atoms. Exemplary alkyls include, but are not limited to methyl, ethyl, n-propyl, isopropyl, n-butyl, isobutyl, s-butyl, t-butyl, pentyl, and hexyl, as well as longer chain alkyls.

The term “alkylene” represents a bivalent straight or branched hydrocarbon group. As non-limiting examples, the terms (C₁-C₂₅) alkylene-aryl and (C₁-C₂₀) alkylene-CO₂R⁴ are intended to represent groups having one or more groups as recited which may be the same or different, at one or more carbon atoms of an alkylene moiety containing from, for the purposes of illustration, 1 to 25, or 1 to 20, carbon atoms, which are straight or branched carbon moieties.

“Alkoxy” represents a group containing an alkyl moiety, defined hereinabove, attached through an oxygen linking atom. As an example, the term “(C₁-C₆) alkoxy” represents a straight- or branched-chain hydrocarbon group having at least 1 and up to 4 carbon atoms attached through an oxygen linking atom. In this particular instance, exemplary “(C₁-C₆)alkoxy” groups include, but are not limited to, methoxy, ethoxy, n-propoxy, isopropoxy, n-butoxy, s-butoxy, isobutoxy, and t-butoxy.

The term “aryl” represents a monocyclic or fused bicyclic group having e.g., 6 to 14 carbon atoms (e.g., (C₆ to Ca) aryl) and having at least one aromatic ring that complies with Hückel's Rule. Examples of “aryl” groups are phenyl (C₆), naphthyl, indenyl, dihydroindenyl, anthracenyl, and phenanthrenyl.

The terms “halogen” and “halo” represent a chloro, fluoro, bromo, or iodo substituent.

The term “oxo” represents a double-bonded oxygen moiety; for example, if attached directly to a carbon atom forms a carbonyl moiety (C═O).

The term “pharmaceutically acceptable” represents those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

The term “pharmaceutically acceptable salts” represents salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively. Non-limiting examples include without limitation sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The salt may precipitate from solution and be collected by filtration or may be recovered by evaporation of the solvent. The degree of ionization in the salt may vary from completely ionized to almost non-ionized. Lists of suitable salts are found in Remington's Pharmaceutical Sciences, 17th ed., Mack Publishing Company, Easton, Pa., 1985, p. 1418, the disclosure of which is hereby incorporated by reference only with regards to the lists of suitable salts.

The terms prevention or “preventing” a disease in a patient refers to the prophylactic administration of a drug to substantially diminish the likelihood or severity of a disorder or biological manifestation thereof, or to delay the onset of such disorder or biological manifestation thereof.

The term “treatment” refers to alleviating the specified condition, eliminating or reducing one or more symptoms of the condition, slowing or eliminating the progression of the condition, and delaying the reoccurrence of the condition in a previously afflicted or diagnosed patient or subject.

The term “heteroatom” means nitrogen, oxygen, or sulfur and includes any oxidized form of phosphorus, nitrogen, such as N(O) {N⁺—O⁻} and sulfur such as S(O) and S(O)₂, and the quaternized form of any basic nitrogen.

The terms“patient” or “subject” refers to mammals and includes humans and non-human mammals. Most preferably, a “patient” is construed to refer to humans.

In one aspect, there is provided a compound of the formula (I):

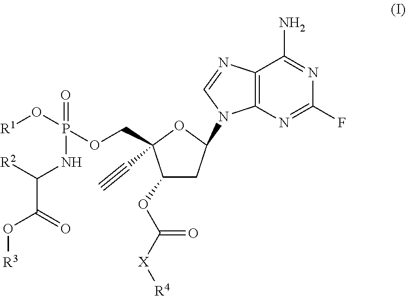

wherein:
R¹ is selected from the group consisting of (C₁-C₂₅) alkyl, (C₁-C₂₅) alkylene-aryl, (C₁-C₂₀) alkylene-CO₂R⁵ and aryl;
R² is selected from the group consisting of (C₁-C₁₀) alkyl; (C₁-C₁₀) alkylene-aryl and aryl;
R³ is selected from the group consisting of (C₁-C₂₅) alkyl, (C₁-C₂₅) alkylene-aryl and aryl; and
R⁴ is selected from the group consisting of (C₁-C₂₅) alkyl, (C₁-C₂₅) alkylene-aryl, (C₁-C₂₀) alkylene-CO₂R⁵ and aryl;
X is single bond, NR⁶, or O
R⁵ is selected from the group consisting of (C₁-C₂₅) alkyl, (C₁-C₂₅) alkylaryl and aryl;
R⁶ is H, (C₁-C₂₅) alkyl, (C₁-C₂₅) alkylaryl, (C₁-C₂₀) alkyl-CO₂R⁵ and aryl; and
wherein each of R¹, R², R³, R⁴, R⁵ and R⁶ may be independently and optionally substituted by one or more (C₁-C₁₄) alkyl, Cl, F, oxo, or (C₁-C₆) alkoxy;

or a pharmaceutically acceptable salt thereof.

In one embodiment, R¹ is (C₆-C₁₄)aryl. More preferably, R¹ is C₆ aryl.

In one embodiment, R¹ is (C₁-C₂₀) alkylene-CO₂R⁵. More preferably, R¹ is selected from (C₁-C₂₀) alkylene-CO₂R⁵, wherein R⁵ is (C₁-C₂₅) alkyl.

In one embodiment, R² is (C₁-C₁₀)alkylene(C₆-C₁₄)aryl. More preferably, R² is C₁ alkylene C₆aryl.

In one embodiment, R² is (C₁-C₁₀)alkyl. In another embodiment, R² is (C₁-C₅)alkyl. More preferably, R² is C₁ alkyl.

In one embodiment, R³ is (C₁-C₂₅)alkyl, e.g., (C₅-C₂₅)alkyl. In another embodiment,
R³ is (C₁-C₄)alkyl. In another embodiment, R³ is (C₁₀-C₁₅)alkyl. In another embodiment, R³ is (C₁₅-C₂₅)alkyl.

In one embodiment, R⁴ is (C₁-C₂₅) alkyl. In another embodiment, R⁴ is (C₅-C₁₀) alkyl. In another embodiment, R⁴ is (C₁₅-C₂₅) alkyl.

In one embodiment, X is a bond.

In one embodiment, X is “O”.

In one embodiment, X is NR⁶

In one embodiment, R¹ is C₆ aryl, R² is C₁ alkyl or C₁ alkylene C₆aryl, R³ is (C₁-C₂₅)alkyl, R⁴ is (C₁-C₂₅ alkyl) and X is a bond.

In one embodiment, R¹ is C₆ aryl, R² is C₁ alkyl or C₁ alkylene C₆aryl, R³ is (C₁-C₂₅)alkyl, R⁴ is (C₁-C₂₅ alkyl) and X is O.

In one embodiment, R¹ is C₆ aryl, R² is C₁ alkyl or C₁ alkylene C₆aryl, R³ is (C₁-C₂₅)alkyl, R⁴ is (C₁-C₂₅ alkyl) and X is NR⁶.

In one embodiment, R¹ is (C₁-C₂₅)alkylene-CO₂R⁵, R² is selected from C₁ alkyl and C₁ alkylene C₆ aryl, R³ is (C₁-C₂₅)alkyl, R⁴ is (C₁-C₂₅ alkyl) and X is a bond.

In one embodiment, R¹ is (C₁-C₂₅)alkylene-CO₂R⁵, R² is selected from C₁ alkyl and C₁ alkylene C₆ aryl, R³ is (C₁-C₂₅)alkyl, R⁴ is (C₁-C₂₅ alkyl) and X is O.

In one embodiment, R¹ is (C₁-C₂₅)alkylene-CO₂R⁵, R² is selected from C₁ alkyl and C₁ alkylene C₆ aryl, R³ is (C₁-C₂₅)alkyl, R⁴ is (C₁-C₂₅ alkyl) and X is NR⁶.

In one embodiment, R² is (C₁-C₁₀)alkyl.

In one embodiment, R² is (C₁-C₅)alkyl.

In additional to that set forth above, in further embodiments, the compounds of the present invention may be optionally substituted by one or more substituents as set forth below. For example, in one embodiment, each of R¹, R², R³, R⁴, R⁵ and R⁶ may be independently and optionally substituted by one or more (C₁-C₆) alkyl, Cl, F, oxo, or (C₁-C₆) alkoxy. Preferably, as an example, each of the aryl groups may be optionally substituted by one or more substituents from (C₁-C₅) alkyl, Cl, F, oxo, or (C₁-C₆) alkoxy.

As an example, in one embodiment, R² is C₁ alkylene C₆aryl, wherein C₆aryl may be optionally substituted by one or more (C₁-C₁₄) alkyl, C₁, or F. More specifically, C₆ aryl may be optionally substituted by one or more F; e.g., two F. As a preferred example: C₆ aryl is

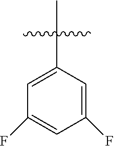 [SMILES: *c1cc(F)cc(F)c1]

In another aspect of the present invention, the invention may encompass various individual compounds. As an example, such specific compounds may be selected from the group consisting of Table 1:

TABLE 1

| Exam- | | |
|---|---|---|
| ple | Structure | Chemical Name |
| 1 | 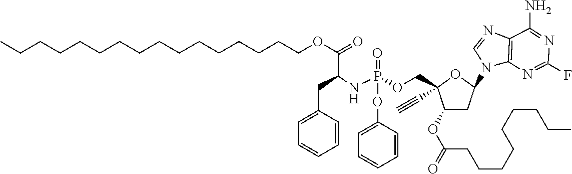 [SMILES: C#C[C@]1(CO[P@@](=O)(N[C@@H](Cc2ccccc2)C(=O)OCCCCCCCCCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((((S)- 1-(hexadecyloxy)-1-oxo-3- phenylpropan-2- yl)amino)(phenoxy)phosphoryl)oxy) methyl)tetrahydrofuran-3-yl decanoate |
| 2 | 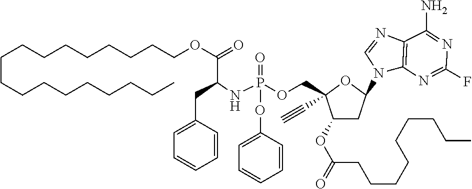 [SMILES: C#C[C@]1(COP(=O)(N[C@@H](Cc2ccccc2)C(=O)OCCCCCCCCCCCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((((S)- 1-(octadecyloxy)-1-oxo-3- phenylpropan-2- yl)amino)(phenoxy)phosphoryl)oxy) methyl)tetrahydrofuran-3-yl decanoate |
| 3 | 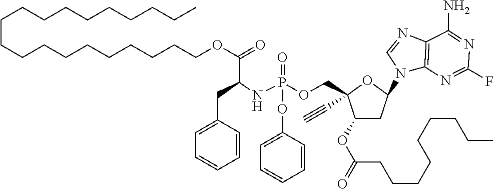 [SMILES: C#C[C@]1(COP(=O)(N[C@@H](Cc2ccccc2)C(=O)OCCCCCCCCCCCCCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((((S)- 1-(icosyloxy)-1-oxo-3- phenylpropan-2- yl)amino)(phenoxy)phosphoryl)oxy) methyl)tetrahydrofuran-3-yl decanoate |
| 4 | 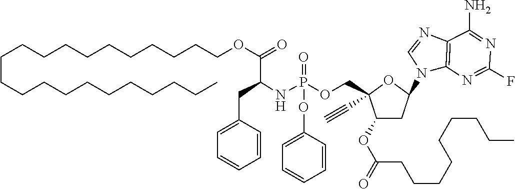 [SMILES: C#C[C@]1(COP(=O)(N[C@@H](Cc2ccccc2)C(=O)OCCCCCCCCCCCCCCCCCCCCCC)Oc2ccccc2)O[C@@H](n2cnc3c(N)nc(F)nc32)C[C@@H]1OC(=O)CCCCCCCCC] | (2R,3S,5R)-5-(6-amino-2-fluoro- 9H-purin-9-yl)-2-((((((S)-1- (docosyloxy)-1-oxo-3- phenylpropan-2- yl)amino)(phenoxy)phosphoryl)oxy) methyl)-2-ethynyltetrahydrofuran-3- yl decanoate |
| 5 | 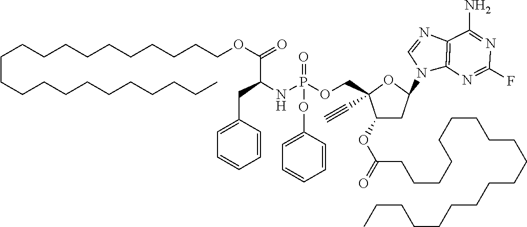 [SMILES: C#C[C@]1(COP(=O)(N[C@@H](Cc2ccccc2)C(=O)OCCCCCCCCCCCCCCCCCCCCCC)Oc2ccccc2)O[C@@H](n2cnc3c(N)nc(F)nc32)C[C@@H]1OC(=O)CCCCCCCCCCCCCCCCCCC] | 2R,3S,5R)-5-(6-amino-2-fluoro-9H- purin-9-yl)-2-((((((S)-1- (docosyloxy)-1-oxo-3- phenylpropan-2- yl)amino)(phenoxy)phosphoryl)oxy) methyl)-2-ethynyltetrahydrofuran-3- yl icosanoate |
| 6 | 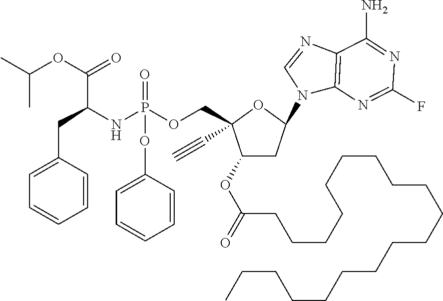 [SMILES: C#C[C@]1(COP(=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2-((((((S)- 1-isopropoxy-1-oxo-3- phenylpropan-2- yl)amino)(phenoxy)phosphoryl)oxy) methyl)tetrahydrofuran-3-yl icosanoate |
| 7 | 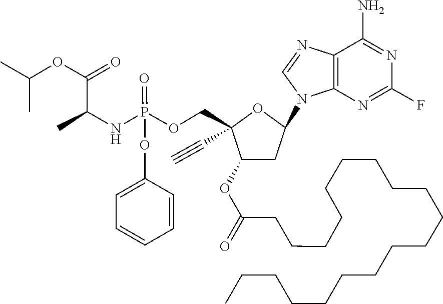 [SMILES: C#C[C@]1(COP(=O)(N[C@@H](C)C(=O)OC(C)C)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2-((((((S)- 1-isopropoxy-1-oxopropan-2- yl)amino)(phenoxy)phosphoryl)oxy) methyl)tetrahydrofuran-3-yl icosanoate |
| 8 | 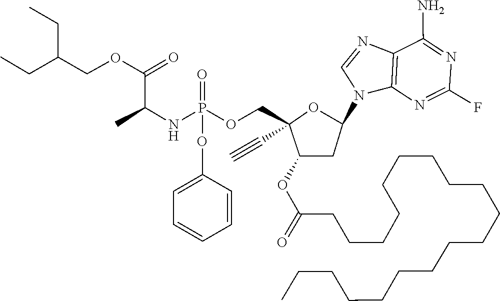 | (2R,3S,5R)-5-(6-amino-2-fluoro- 9H-purin-9-yl)-2-((((((S)-1-(2- ethylbutoxy)-1-oxopropan-2- yl)amino)(phenoxy)phosphoryl)oxy) methyl)-2-ethynyltetrahydrofuran-3- yl icosanoate |
| 9 | 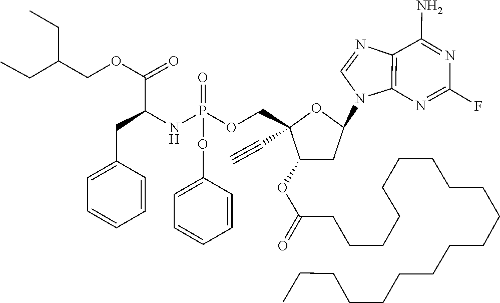 [SMILES: C#C[C@]1(COP(=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-((((((S)-1-(2- ethylbutoxy)-1-oxo-3- phenylpropan-2- yl)amino)(phenoxy)phosphoryl)oxy) methyl)-2-ethynyltetrahydrofuran-3- yl icosanoate |
| 10 | 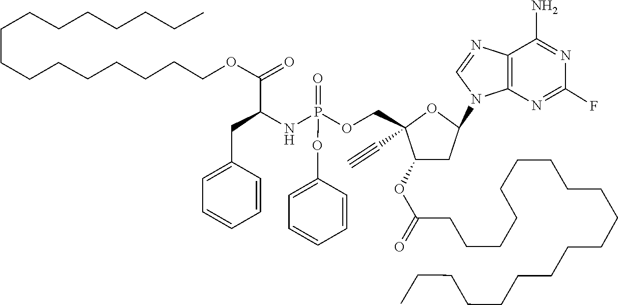 [SMILES: C#C[C@]1(COP(=O)(N[C@@H](Cc2ccccc2)C(=O)OCCCCCCCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2-((((((S)- 1-(hexadecyloxy)-1-oxo-3- phenylpropan-2- yl)amino)(phenoxy)phosphoryl)oxy) methyl)tetrahydrofuran-3-yl icosanoate |
| 11 | 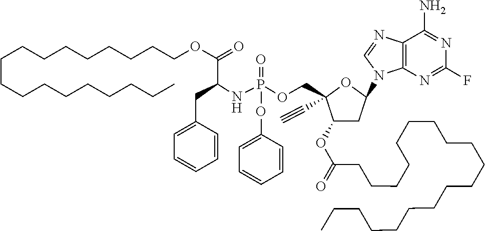 [SMILES: C#C[C@]1(COP(=O)(N[C@@H](Cc2ccccc2)C(=O)OCCCCCCCCCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2-((((((S)- 1-(octadecyloxy)-1-oxo-3- phenylpropan-2- yl)amino)(phenoxy)phosphoryl)oxy) methyl)tetrahydrofuran-3-yl icosanoate |
| 12 | 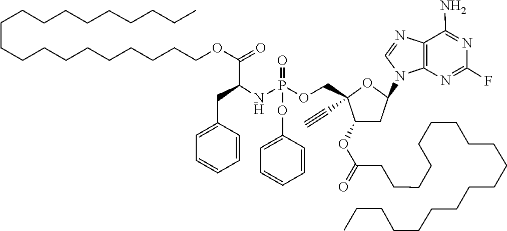 [SMILES: C#C[C@]1(COP(=O)(N[C@@H](Cc2ccccc2)C(=O)OCCCCCCCCCCCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2-((((((S)- 1-(icosyloxy)-1-oxo-3- phenylpropan-2- yl)amino)(phenoxy)phosphoryl)oxy) methyl)tetrahydrofuran-3-yl |
| 13 | 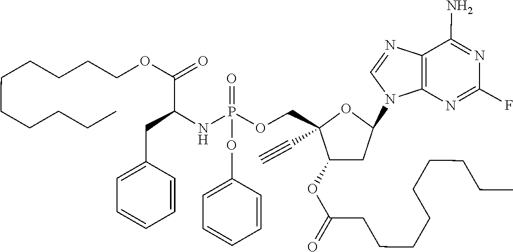 [SMILES: C#C[C@]1(COP(=O)(N[C@@H](Cc2ccccc2)C(=O)OCCCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((((S)-1- (decyloxy)-1-oxo-3-phenylpropan- 2- yl)amino)(phenoxy)phosphoryl)oxy) methyl)-2-ethynyltetrahydrofuran-3- yl decanoate |
| 14 | 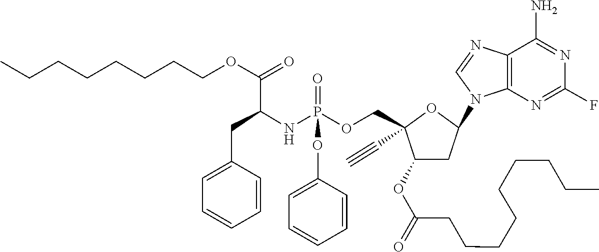 [SMILES: C#C[C@]1(CO[P@@](=O)(N[C@@H](Cc2ccccc2)C(=O)OCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((S)- (((S)-1-(octyloxy)-1-oxo-3- phenylpropan-2- yl)amino)(phenoxy)phosphoryl)oxy) methyl)tetrahydrofuran-3-yl decanoate |
| 15 | 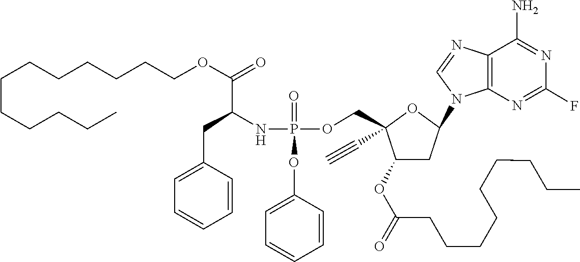 [SMILES: C#C[C@]1(CO[P@@](=O)(N[C@@H](Cc2ccccc2)C(=O)OCCCCCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((S)-(((S)-1- (dodecyloxy)-1-oxo-3- phenylpropan-2- yl)amino)(phenoxy)phosphoryl)oxy) methyl)-2-ethynyltetrahydrofuran-3- yl decanoate |
| 16 | 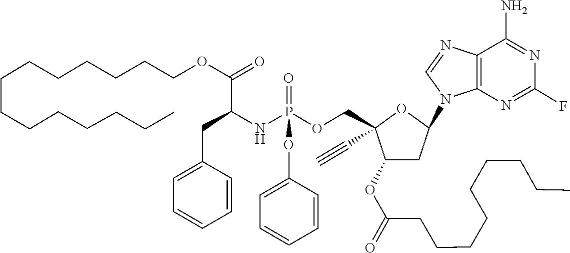 [SMILES: C#C[C@]1(CO[P@@](=O)(N[C@@H](Cc2ccccc2)C(=O)OCCCCCCCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((S)- (((S)-1-oxo-3-phenyl-1- (tetradecyloxy)propan-2- yl)amino)(phenoxy)phosphoryl)oxy) methyl)tetrahydrofuran-3-yl decanoate |
| 17 | 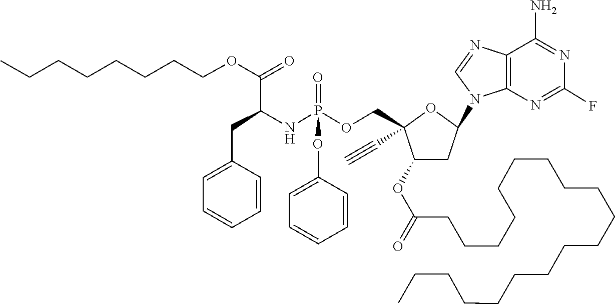 [SMILES: C#C[C@]1(CO[P@@](=O)(N[C@@H](Cc2ccccc2)C(=O)OCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2-((((S)- (((S)-1-(octyloxy)-1-oxo-3- phenylpropan-2- yl)amino)(phenoxy)phosphoryl)oxy) methyl)tetrahydrofuran-3-yl icosanoate |
| 18 | 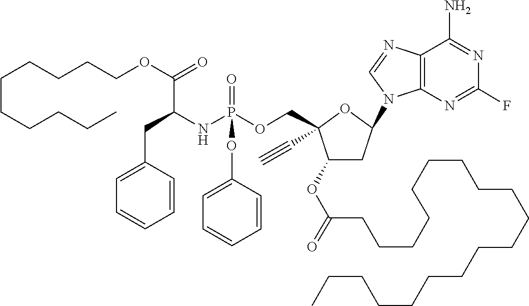 [SMILES: C#C[C@]1(CO[P@@](=O)(N[C@@H](Cc2ccccc2)C(=O)OCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-Amino-2-fluoro- 9H-purin-9-yl)-2-((((S)-(((S)-1- (decyloxy)-1-oxo-3-phenylpropan- 2- yl)amino)(phenoxy)phosphoryl)oxy) methyl)-2-ethynyltetrahydrofuran-3- yl icosanoate |
| 19 | 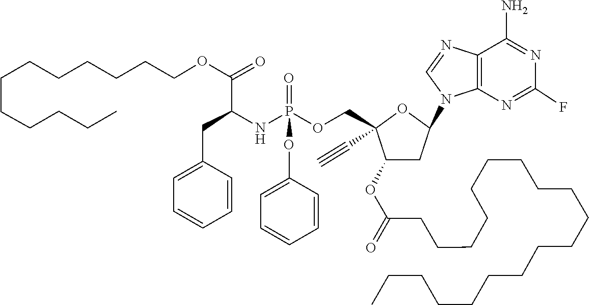 [SMILES: C#C[C@]1(CO[P@@](=O)(N[C@@H](Cc2ccccc2)C(=O)OCCCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-Amino-2-fluoro- 9H-purin-9-yl)-2-((((S)-(((S)-1- (dodecyloxy)-1-oxo-3- phenylpropan-2- yl)amino)(phenoxy)phosphoryl)oxy) methyl)-2-ethynyltetrahydrofuran-3- yl icosanoate |
| 20 | 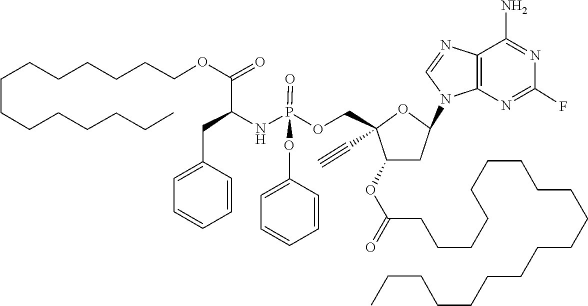 [SMILES: C#C[C@]1(CO[P@@](=O)(N[C@@H](Cc2ccccc2)C(=O)OCCCCCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-Amino-2-fluoro- 9H-purin-9-yl)-2-ethynyl-2-((((S)- (((S)-1-oxo-3-phenyl-1- (tetradecyloxy)propan-2- yl)amino)(phenoxy)phosphoryl)oxy) methyl)tetrahydrofuran-3-yl icosanoate |
| 21 | 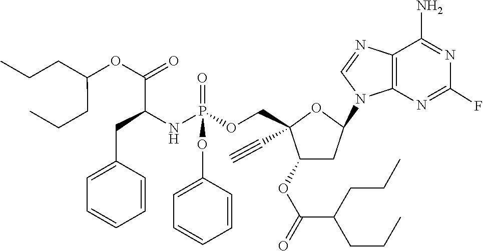 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl 2- propylpentanoate |
| 22 | 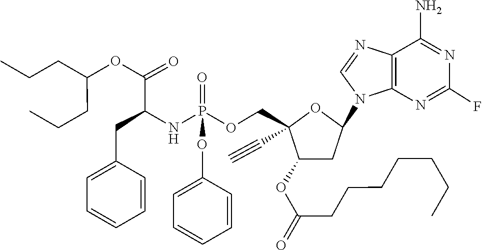 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl octanoate |
| 23 | 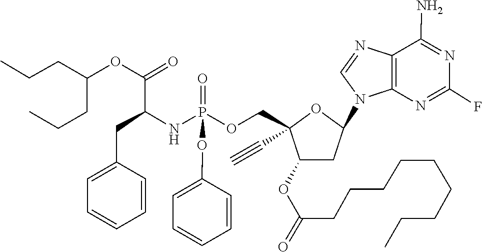 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl decanoate |
| 24 | 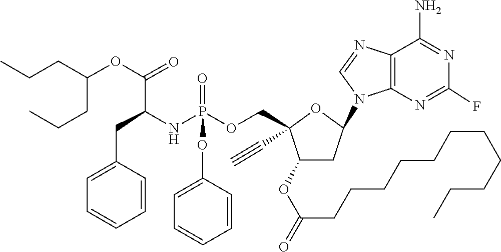 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl dodecanoate |
| 25 | 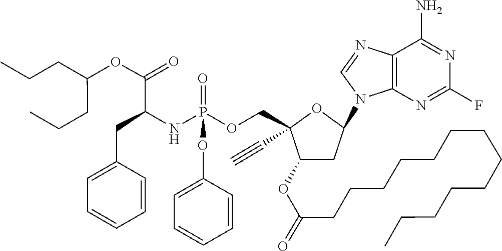 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl tetradecanoate |
| 26 | 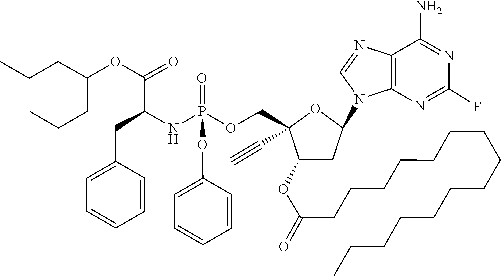 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl palmitate |
| 27 | 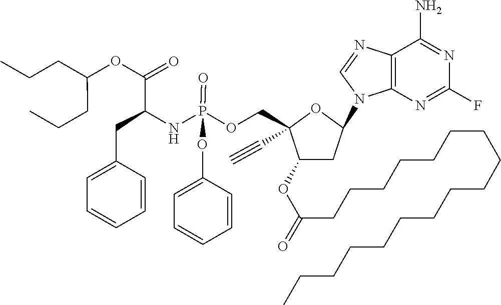 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl stearate |
| 28 | 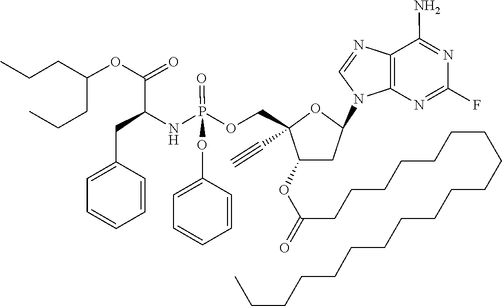 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl icosanoate |
| 29 | 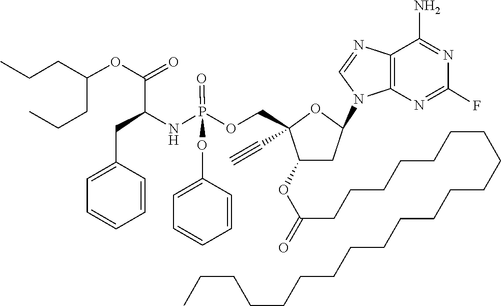 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCCCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl docosanoate |
| 30 | 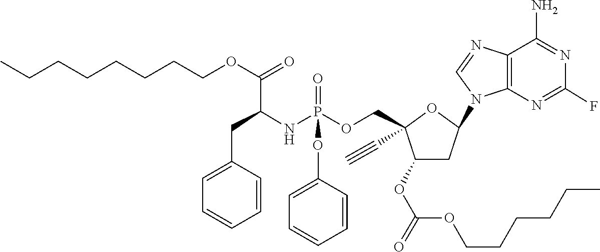 [SMILES: C#C[C@]1(CO[P@@](=O)(N[C@@H](Cc2ccccc2)C(=O)OCCCCCCCC)Oc2ccccc2)O[C@@H](n2cnc3c(N)nc(F)nc32)C[C@@H]1OC(=O)OCCCCCC] | Octyl ((S)-(((2R,3S,5R)-5-(6-amino- 2-fluoro-9H-purin-9-yl)-2-ethynyl-3- (((hexyloxy)carbonyl)oxy)tetrahydro- furan-2- yl)methoxy)(phenoxy)phosphoryl)- L-phenylalaninate |
| 31 | 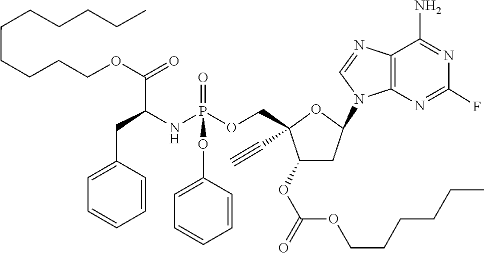 [SMILES: C#C[C@]1(CO[P@@](=O)(N[C@@H](Cc2ccccc2)C(=O)OCCCCCCCCCC)Oc2ccccc2)O[C@@H](n2cnc3c(N)nc(F)nc32)C[C@@H]1OC(=O)OCCCCCC] | Decyl ((S)-(((2R,3S,5R)-5-(6- amino-2-fluoro-9H-purin-9-yl)-2- ethynyl-3- (((hexyloxy)carbonyl)oxy)tetrahydro- furan-2- yl)methoxy)(phenoxy)phosphoryl)- L-phenylalaninate |
| 32 | 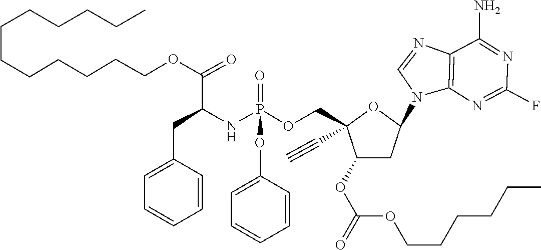 [SMILES: C#C[C@]1(CO[P@@](=O)(N[C@@H](Cc2ccccc2)C(=O)OCCCCCCCCCCCC)Oc2ccccc2)O[C@@H](n2cnc3c(N)nc(F)nc32)C[C@@H]1OC(=O)OCCCCCC] | Dodecyl ((S)-(((2R,3S,5R)-5-(6- amino-2-fluoro-9H-purin-9-yl)-2- ethynyl-3- (((hexyloxy)carbonyl)oxy)tetrahydro- furan-2- yl)methoxy)(phenoxy)phosphoryl)- L-phenylalaninate |
| 33 | 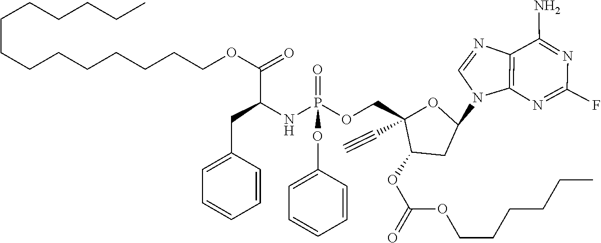 [SMILES: C#C[C@]1(CO[P@@](=O)(N[C@@H](Cc2ccccc2)C(=O)OCCCCCCCCCCCCCC)Oc2ccccc2)O[C@@H](n2cnc3c(N)nc(F)nc32)C[C@@H]1OC(=O)OCCCCCC] | Tetradecyl ((S)-(((2R,3S,5R)-5-(6- amino-2-fluoro-9H-purin-9-yl)-2- ethynyl-3- (((hexyloxy)carbonyl)oxy)tetrahydro- furan-2- yl)methoxy)(phenoxy)phosphoryl)- L-phenylalaninate |
| 34 | 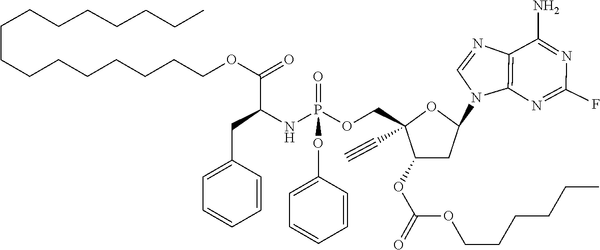 [SMILES: C#C[C@]1(CO[P@@](=O)(N[C@@H](Cc2ccccc2)C(=O)OCCCCCCCCCCCCCCCC)Oc2ccccc2)O[C@@H](n2cnc3c(N)nc(F)nc32)C[C@@H]1OC(=O)OCCCCCC] | Hexadecyl ((S)-(((2R,3S,5R)-5-(6- amino-2-fluoro-9H-purin-9-yl)-2- ethynyl-3- (((hexyloxy)carbonyl)oxy)tetrahydro- furan-2- yl)methoxy)(phenoxy)phosphoryl)- L-phenylalaninate |
| 35 | 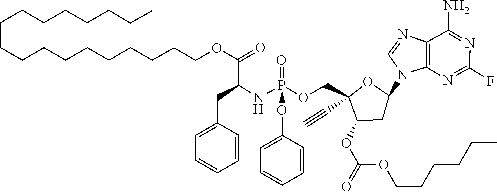 [SMILES: C#C[C@]1(CO[P@@](=O)(N[C@@H](Cc2ccccc2)C(=O)OCCCCCCCCCCCCCCCCCC)Oc2ccccc2)O[C@@H](n2cnc3c(N)nc(F)nc32)C[C@@H]1OC(=O)OCCCCCC] | Octadecyl ((S)-(((2R,3S,5R)-5-(6- amino-2-fluoro-9H-purin-9-yl)-2- ethynyl-3- (((hexyloxy)carbonyl)oxy)tetrahydro- furan-2- yl)methoxy)(phenoxy)phosphoryl)- L-phenylalaninate |
| 36 | 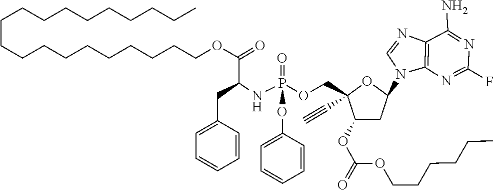 [SMILES: C#C[C@]1(CO[P@@](=O)(N[C@@H](Cc2ccccc2)C(=O)OCCCCCCCCCCCCCCCCCCCC)Oc2ccccc2)O[C@@H](n2cnc3c(N)nc(F)nc32)C[C@@H]1OC(=O)OCCCCCC] | Icosyl ((S)-(((2R,3S,5R)-5-(6- amino-2-fluoro-9H-purin-9-yl)-2- ethynyl-3- (((hexyloxy)carbonyl)oxy)tetrahydro- furan-2- yl)methoxy)(phenoxy)phosphoryl)- L-phenylalaninate |
| 37 | 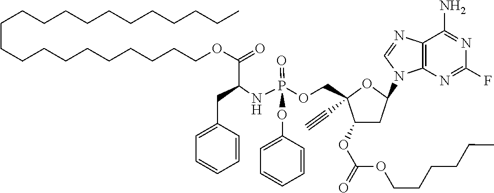 [SMILES: C#C[C@]1(CO[P@@](=O)(N[C@@H](Cc2ccccc2)C(=O)OCCCCCCCCCCCCCCCCCCCCCC)Oc2ccccc2)O[C@@H](n2cnc3c(N)nc(F)nc32)C[C@@H]1OC(=O)OCCCCCC] | Docosyl ((S)-(((2R,3S,5R)-5-(6- amino-2-fluoro-9H-purin-9-yl)-2- ethynyl-3- (((hexyloxy)carbonyl)oxy)tetrahydro- furan-2- yl)methoxy)(phenoxy)phosphoryl)- L-phenylalaninate |
| 38 | 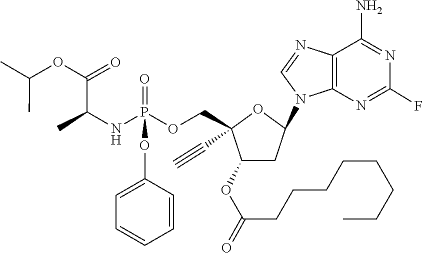 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl nonanoate |
| 39 | 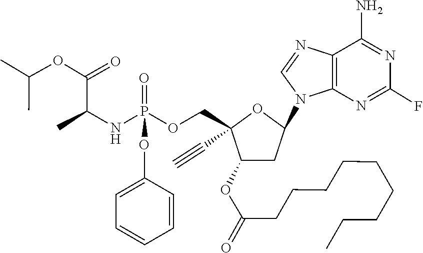 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl decanoate |
| 40 | 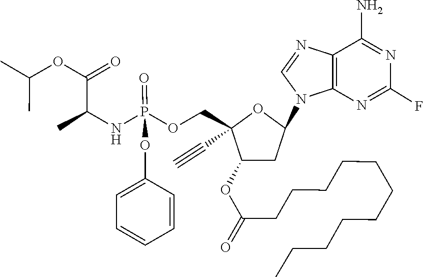 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl dodecanoate |
| 41 | 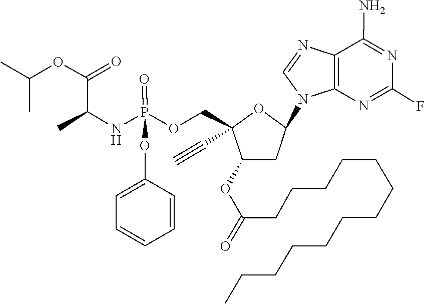 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl tetradecanoate |
| 42 | 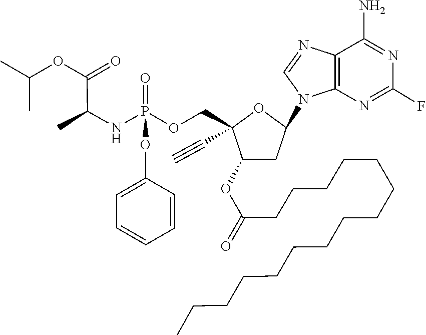 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl palmitate |
| 43 | 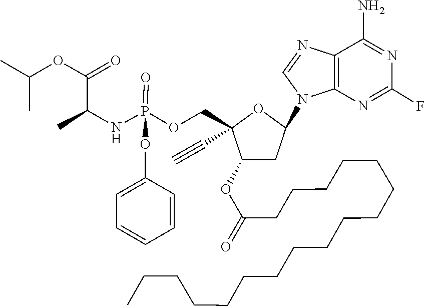 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl stearate |
| 44 | 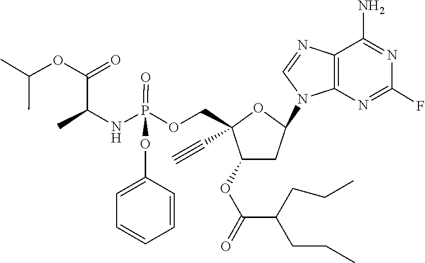 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl 2- propylpentanoate |
| 45 | 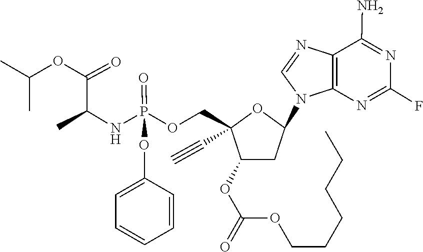 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OCCCCCC] | Isopropyl ((S)-(((2R,3S,5R)-5-(6- amino-2-fluoro-9H-purin-9-yl)-2- ethynyl-3- (((hexyloxy)carbonyl)oxy)tetrahydro- furan-2- yl)methoxy)(phenoxy)phosphoryl)- L-alaninate |
| 46 | 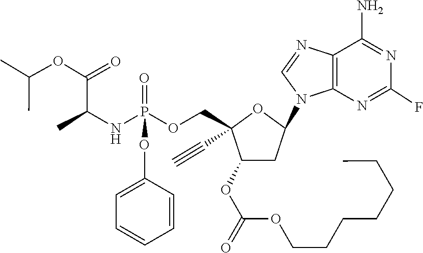 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OCCCCCCC] | Isopropyl ((S)-(((2R,3S,5R)-5-(6- amino-2-fluoro-9H-purin-9-yl)-2- ethynyl-3- (((heptyloxy)carbonyl)oxy)tetrahydro- furan-2- yl)methoxy)(phenoxy)phosphoryl)- L-alaninate |
| 47 | 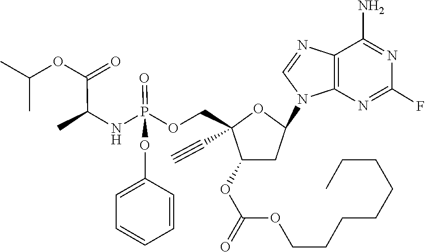 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OCCCCCCCC] | Isopropyl ((S)-(((2R,3S,5R)-5-(6- amino-2-fluoro-9H-purin-9-yl)-2- ethynyl-3- (((octyloxy)carbonyl)oxy)tetrahydro- furan-2- yl)methoxy)(phenoxy)phosphoryl)- L-alaninate |
| 48 | 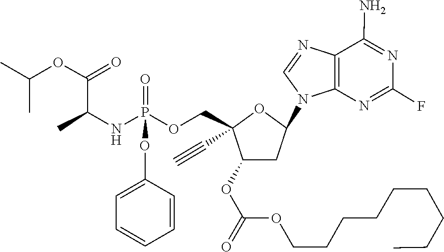 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OCCCCCCCCC] | Isopropyl ((S)-(((2R,3S,5R)-5-(6- amino-2-fluoro-9H-purin-9-yl)-2- ethynyl-3- (((nonyloxy)carbonyl)oxy)tetrahydro- furan-2- yl)methoxy)(phenoxy)phosphoryl)- L-alaninate |
| 49 | 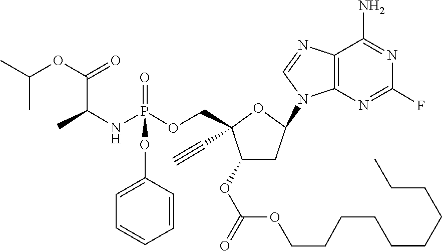 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OCCCCCCCCCC] | Isopropyl ((S)-(((2R,3S,5R)-5-(6- amino-2-fluoro-9H-purin-9-yl)-3- (((decyloxy)carbonyl)oxy)-2- ethynyltetrahydrofuran-2- yl)methoxy)(phenoxy)phosphoryl)- L-alaninate |
| 50 | 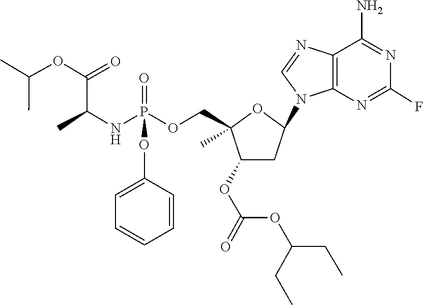 [SMILES: CCC(CC)OC(=O)O[C@H]1C[C@H](n2cnc3c(N)nc(F)nc32)O[C@]1(C)CO[P@@](=O)(N[C@@H](C)C(=O)OC(C)C)Oc1ccccc1] | Isopropyl ((S)-(((2R,3S,5R)-5-(6- amino-2-fluoro-9H-purin-9-yl)-2- ethynyl-3-(((pentan-3- yloxy)carbonyl)oxy)tetrahydrofuran- 2- yl)methoxy)(phenoxy)phosphoryl)- L-alaninate |
| 51 | 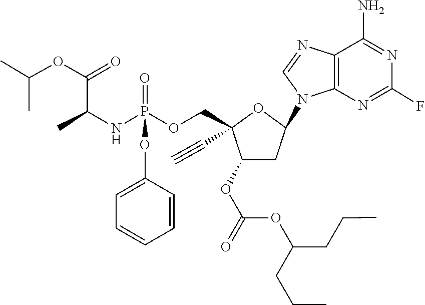 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OC(CCC)CCC] | Isopropyl ((S)-(((2R,3S,5R)-5-(6- amino-2-fluoro-9H-purin-9-yl)-2- ethynyl-3-(((heptan-4- yloxy)carbonyl)oxy)tetrahydrofuran- 2- yl)methoxy)(phenoxy)phosphoryl)- L-alaninate |
| 52 | 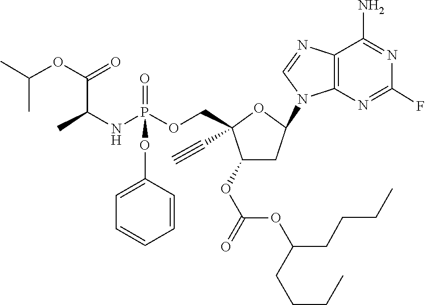 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OC(CCCC)CCCC] | Isopropyl ((S)-(((2R,3S,5R)-5-(6- amino-2-fluoro-9H-purin-9-yl)-2- ethynyl-3-(((nonan-5- yloxy)carbonyl)oxy)tetrahydrofuran- 2- yl)methoxy)(phenoxy)phosphoryl)- L-alaninate |
| 53 | 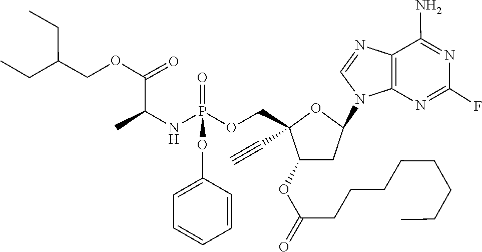 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran-3- yl nonanoate |
| 54 | 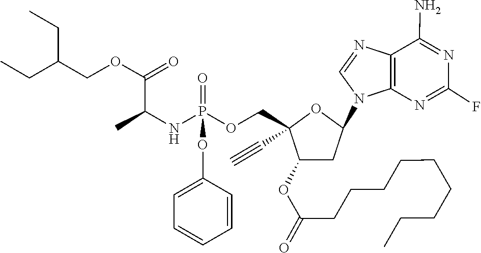 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran-3- yl decanoate |
| 55 | 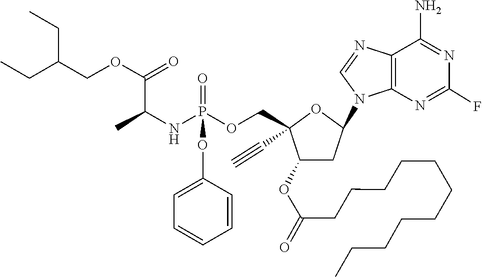 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran-3- yl dodecanoate |
| 56 | 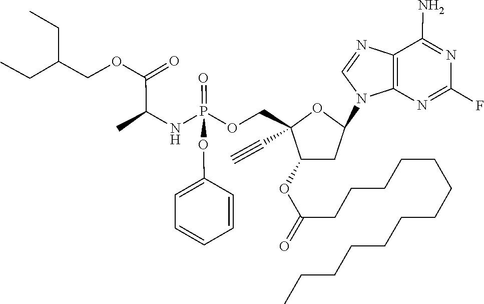 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran-3- yl tetradecanoate |
| 57 | 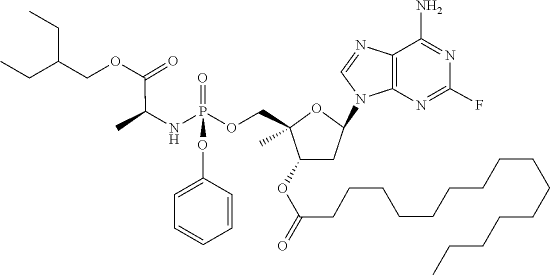 [SMILES: CCCCCCCCCCCCCCCC(=O)O[C@H]1C[C@H](n2cnc3c(N)nc(F)nc32)O[C@]1(C)CO[P@@](=O)(N[C@@H](C)C(=O)OCC(CC)CC)Oc1ccccc1] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran-3- yl palmitate |
| 58 | 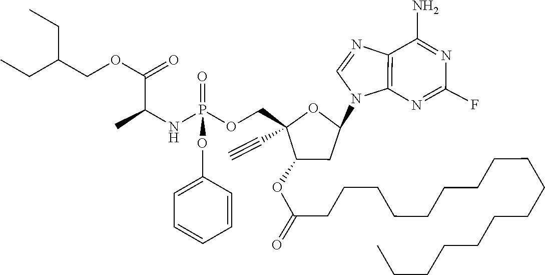 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCCCCCCCCCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran-3- yl stearate |
| 59 | 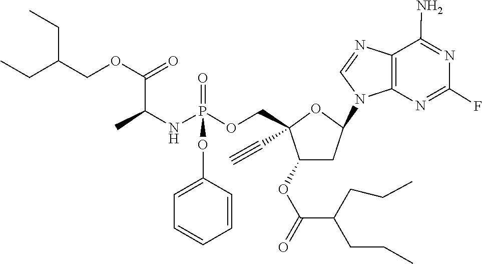 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran-3- yl 2-propylpentanoate |
| 60 | 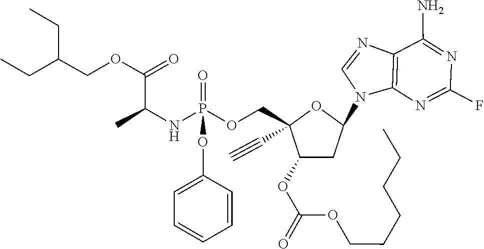 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCC] | 2-Ethylbutyl ((S)-(((2R,3S,5R)-5-(6- amino-2-fluoro-9H-purin-9-yl)-2- ethynyl-3- (((hexyloxy)carbonyl)oxy)tetrahydro- furan-2- yl)methoxy)(phenoxy)phosphoryl)- L-alaninate |
| 61 | 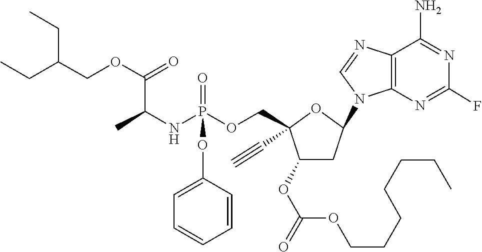 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCC] | 2-Ethylbutyl ((S)-(((2R,3S,5R)-5-(6- amino-2-fluoro-9H-purin-9-yl)-2- ethynyl-3- (((heptyloxy)carbonyl)oxy)tetrahydro- furan-2- yl)methoxy)(phenoxy)phosphoryl)- L-alaninate |
| 62 | 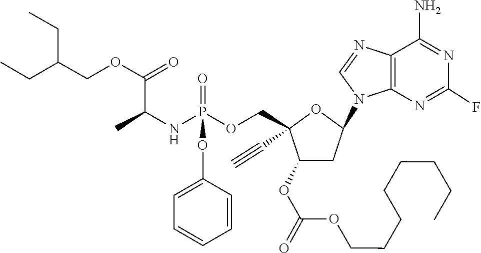 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCCC] | 2-Ethylbutyl ((S)-(((2R,3S,5R)-5-(6- amino-2-fluoro-9H-purin-9-yl)-2- ethynyl-3- (((octyloxy)carbonyl)oxy)tetrahydro- furan-2- yl)methoxy)(phenoxy)phosphoryl)- L-alaninate |
| 63 | 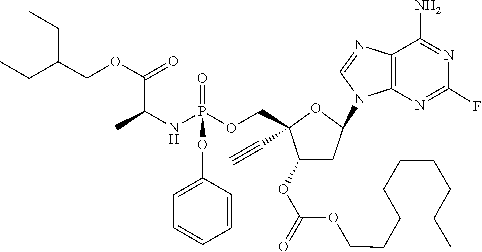 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCCCC] | 2-Ethylbutyl ((S)-(((2R,3S,5R)-5-(6- amino-2-fluoro-9H-purin-9-yl)-2- ethynyl-3- (((nonyloxy)carbonyl)oxy)tetrahydro- furan-2- yl)methoxy)(phenoxy)phosphoryl)- L-alaninate |
| 64 | 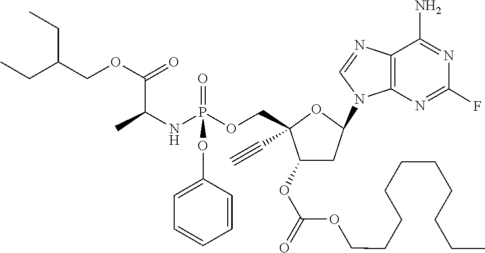 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCCCCC] | 2-Ethylbutyl ((S)-(((2R,3S,5R)-5-(6- amino-2-fluoro-9H-purin-9-yl)-3- (((decyloxy)carbonyl)oxy)-2- ethynyltetrahydrofuran-2- yl)methoxy)(phenoxy)phosphoryl)- L-alaninate |
| 65 | 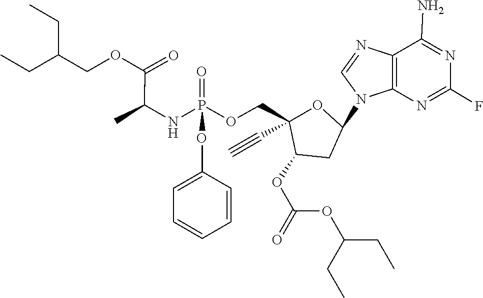 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OC(CC)CC] | 2-Ethylbutyl ((S)-(((2R,3S,5R)-5-(6- amino-2-fluoro-9H-purin-9-yl)-2- ethynyl-3-(((pentan-3- yloxy)carbonyl)oxy)tetrahydrofuran- 2- yl)methoxy)(phenoxy)phosphoryl)- L-alaninate |
| 66 | 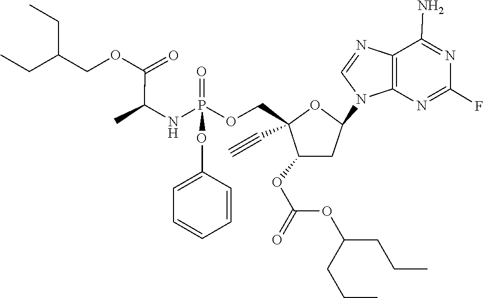 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OC(CCC)CCC] | 2-Ethylbutyl ((S)-(((2R,3S,5R)-5-(6- amino-2-fluoro-9H-purin-9-yl)-2- ethynyl-3-(((heptan-4- yloxy)carbonyl)oxy)tetrahydrofuran- 2- yl)methoxy)(phenoxy)phosphoryl)- L-alaninate |
| 67 | 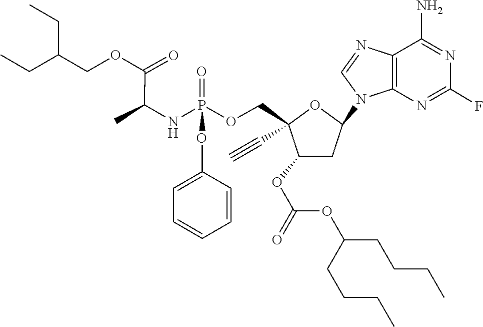 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OC(CCCC)CCCC] | 2-Ethylbutyl ((S)-(((2R,3S,5R)-5-(6- amino-2-fluoro-9H-purin-9-yl)-2- ethynyl-3-(((nonan-5- yloxy)carbonyl)oxy)tetrahydrofuran- 2- yl)methoxy)(phenoxy)phosphoryl)- L-alaninate |
| 68 | 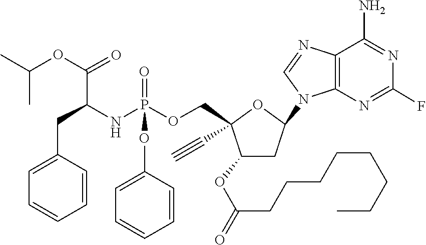 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl nonanoate |
| 69 | 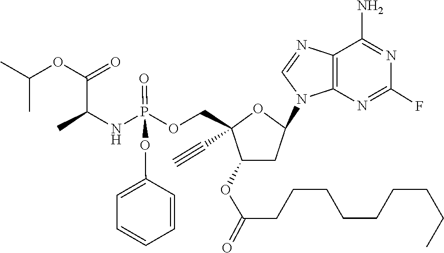 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl decanoate |
| 70 | 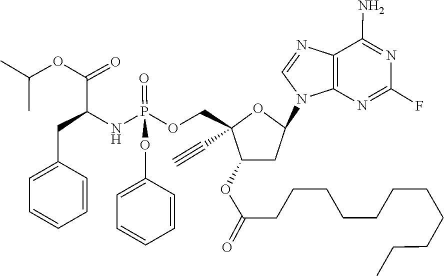 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCC] | (2R,3S,5R)-5-(6-amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl dodecanoate |
| 71 | 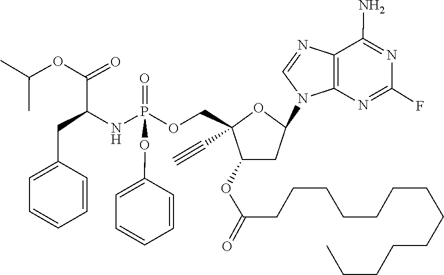 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl tetradecanoate |
| 72 | 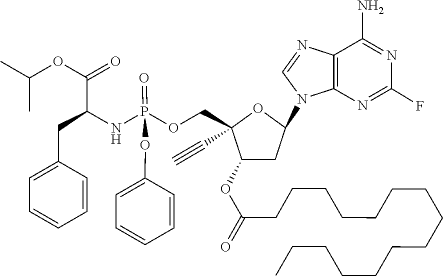 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl palmitate |
| 73 | 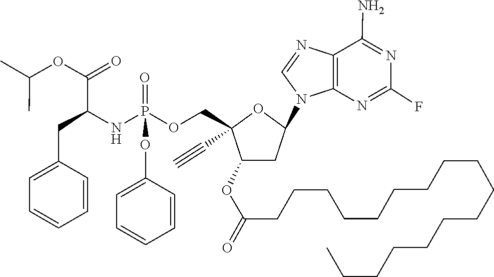 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCCCCCCCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl stearate |
| 74 | 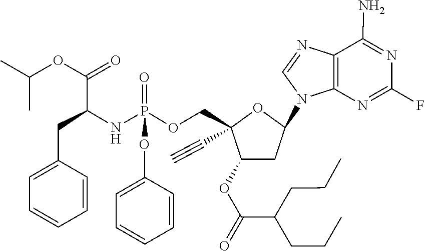 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl 2- propylpentanoate |
| 75 | 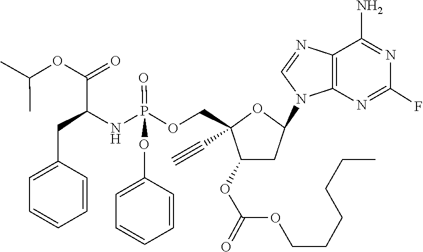 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCC] | Isopropyl ((S)-(((2R,3S,5R)-5-(6- amino-2-fluoro-9H-purin-9-yl)-2- ethynyl-3- (((hexyloxy)carbonyl)oxy)tetrahydro- furan-2- yl)methoxy)(phenoxy)phosphoryl)- L-phenylalaninate |
| 76 | 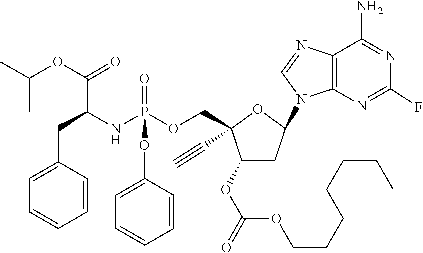 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCC] | Isopropyl ((S)-(((2R,3S,5R)-5-(6- amino-2-fluoro-9H-purin-9-yl)-2- ethynyl-3- (((heptyloxy)carbonyl)oxy)tetrahydro- furan-2- yl)methoxy)(phenoxy)phosphoryl)- L-phenylalaninate |
| 77 | 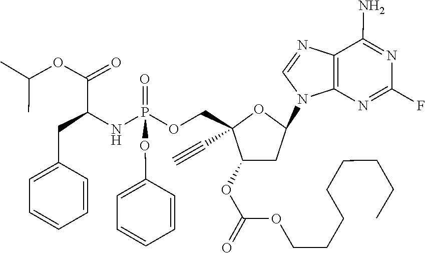 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCCC] | Isopropyl ((S)-(((2R,3S,5R)-5-(6- amino-2-fluoro-9H-purin-9-yl)-2- ethynyl-3- (((octyloxy)carbonyl)oxy)tetrahydro- furan-2- yl)methoxy)(phenoxy)phosphoryl)- L-phenylalaninate |
| 78 | 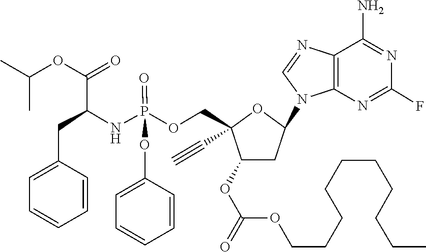 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCCCCC] | Isopropyl ((S)-(((2R,3S,5R)-5-(6- amino-2-fluoro-9H-purin-9-yl)-3- (((decyloxy)carbonyl)oxy)-2- ethynyltetrahydrofuran-2- yl)methoxy)(phenoxy)phosphoryl)- L-phenylalaninat |
| 79 | 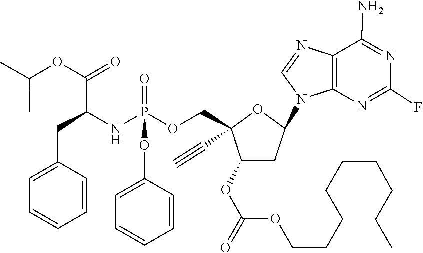 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCCCC] | Isopropyl ((S)-(((2R,3S,5R)-5-(6- amino-2-fluoro-9H-purin-9-yl)-2- ethynyl-3- (((nonyloxy)carbonyl)oxy)tetrahydro- furan-2- yl)methoxy)(phenoxy)phosphoryl)- L-phenylalaninate |
| 80 | 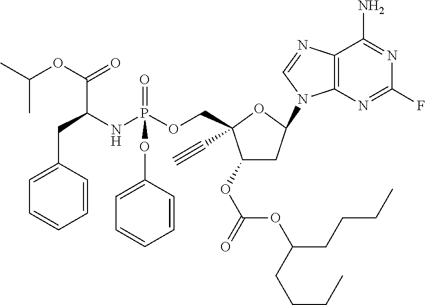 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OC(CCCC)CCCC] | Isopropyl ((S)-(((2R,3S,5R)-5-(6- amino-2-fluoro-9H-purin-9-yl)-2- ethynyl-3-(((nonan-5- yloxy)carbonyl)oxy)tetrahydrofuran- 2- yl)methoxy)(phenoxy)phosphoryl)- L-phenylalaninate |
| 81 | 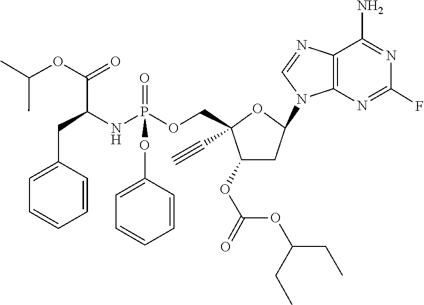 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OC(CC)CC] | Isopropyl ((S)-(((2R,3S,5R)-5-(6- amino-2-fluoro-9H-purin-9-yl)-2- ethynyl-3-(((pentan-3- yloxy)carbonyl)oxy)tetrahydrofuran- 2- yl)methoxy)(phenoxy)phosphoryl)- L-phenylalaninate |
| 82 | 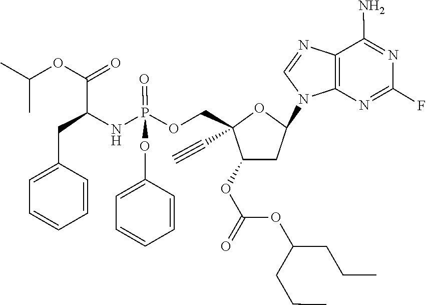 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OC(CCC)CCC] | Isopropyl ((S)-(((2R,3S,5R)-5-(6- amino-2-fluoro-9H-purin-9-yl)-2- ethynyl-3-(((heptan-4- yloxy)carbonyl)oxy)tetrahydrofuran- 2- yl)methoxy)(phenoxy)phosphoryl)- L-phenylalaninate |
| 83 | 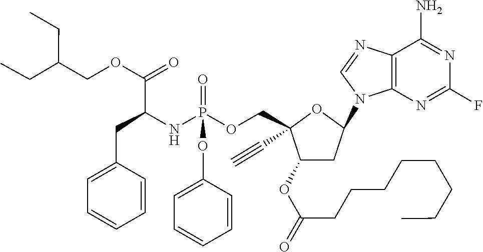 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran-3- yl nonanoate |
| 84 | 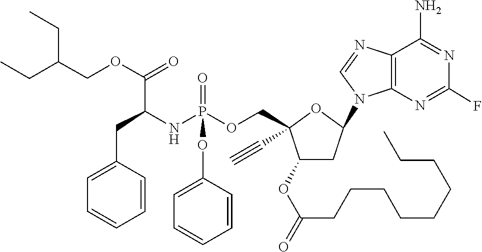 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran-3- yl decanoate |
| 85 | 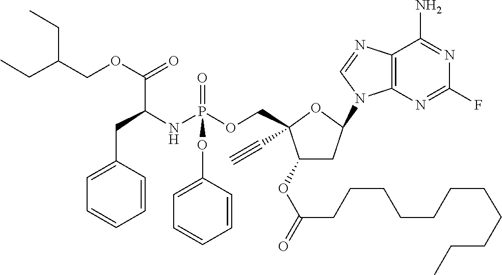 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran-3- yl dodecanoate |
| 86 | 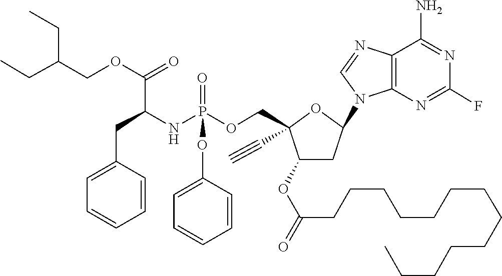 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran-3- yl tetradecanoate |
| 87 | 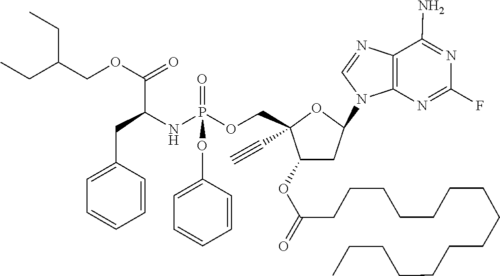 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran-3- yl palmitate |
| 88 | 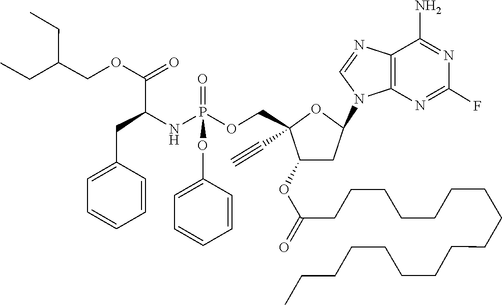 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran-3- yl stearate |
| 89 | 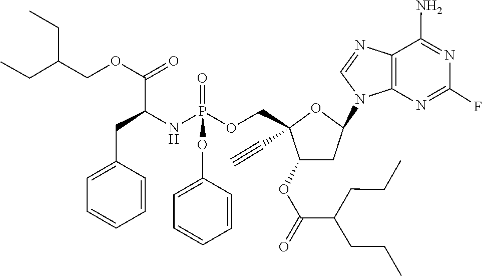 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran-3- yl 2-propylpentanoate |
| 90 | 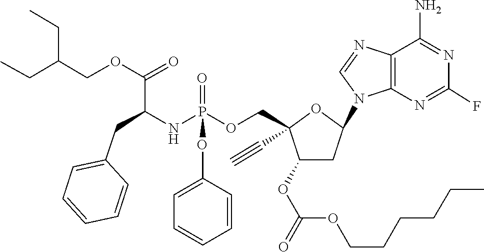 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCC] | 2-Ethylbutyl ((S)-(((2R,3S,5R)-5-(6- amino-2-fluoro-9H-purin-9-yl)-2- ethynyl-3- (((hexyloxy)carbonyl)oxy)tetrahydro- furan-2- yl)methoxy)(phenoxy)phosphoryl)- L-phenylalaninate |
| 91 | 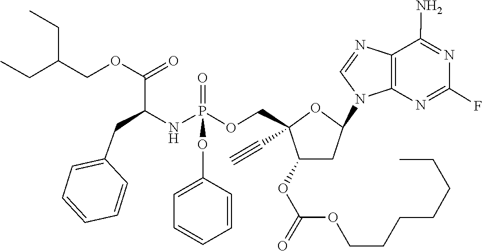 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCC] | 2-Ethylbutyl ((S)-(((2R,3S,5R)-5-(6- amino-2-fluoro-9H-purin-9-yl)-2- ethynyl-3- (((heptyloxy)carbonyl)oxy)tetrahydro- furan-2- yl)methoxy)(phenoxy)phosphoryl)- L-phenylalaninate |
| 92 | 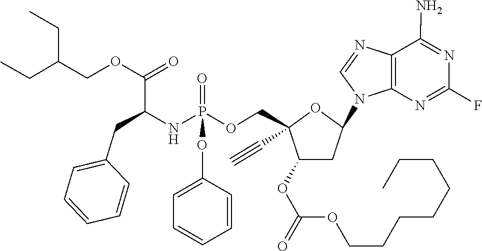 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCCC] | 2-Ethylbutyl ((S)-(((2R,3S,5R)-5-(6- amino-2-fluoro-9H-purin-9-yl)-2- ethynyl-3- (((octyloxy)carbonyl)oxy)tetrahydro- furan-2- yl)methoxy)(phenoxy)phosphoryl)- L-phenylalaninate |
| 93 | 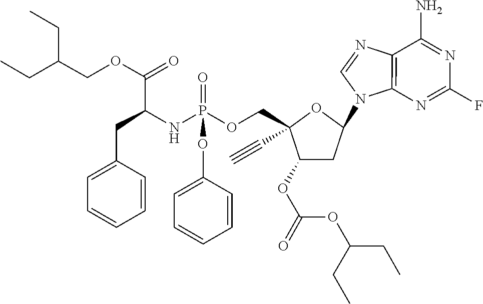 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OC(CC)CC] | 2-Ethylbutyl ((S)-(((2R,3S,5R)-5-(6- amino-2-fluoro-9H-purin-9-yl)-2- ethynyl-3-(((pentan-3- yloxy)carbonyl)oxy)tetrahydrofuran- 2- yl)methoxy)(phenoxy)phosphoryl)- L-phenylalaninate |
| 94 | 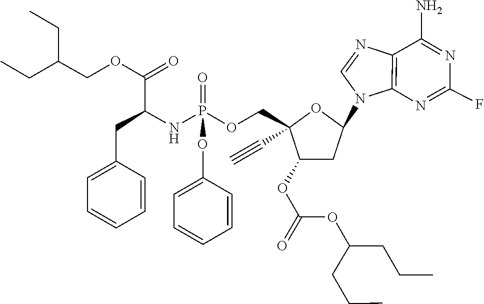 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OC(CCC)CCC] | 2-Ethylbutyl ((S)-(((2R,3S,5R)-5-(6- amino-2-fluoro-9H-purin-9-yl)-2- ethynyl-3-(((heptan-4- yloxy)carbonyl)oxy)tetrahydrofuran- 2- yl)methoxy)(phenoxy)phosphoryl)- L-phenylalaninate |
| 95 | 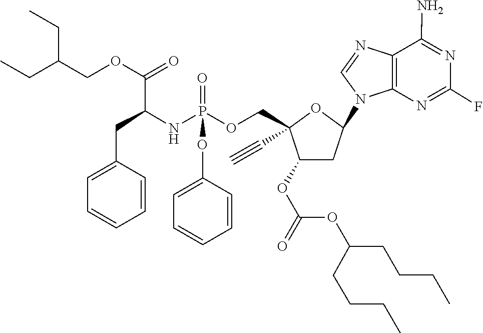 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OC(CCCC)CCCC] | 2-Ethylbutyl ((S)-(((2R,3S,5R)-5-(6- amino-2-fluoro-9H-purin-9-yl)-2- ethynyl-3-(((nonan-5- yloxy)carbonyl)oxy)tetrahydrofuran- 2- yl)methoxy)(phenoxy)phosphoryl)- L-phenylalaninate |
| 96 | 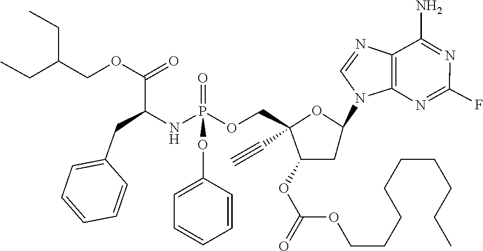 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCCCC] | 2-Ethylbutyl ((S)-(((2R,3S,5R)-5-(6- amino-2-fluoro-9H-purin-9-yl)-2- ethynyl-3- (((nonyloxy)carbonyl)oxy)tetrahydro- furan-2- yl)methoxy)(phenoxy)phosphoryl)- L-phenylalaninate |
| 97 | 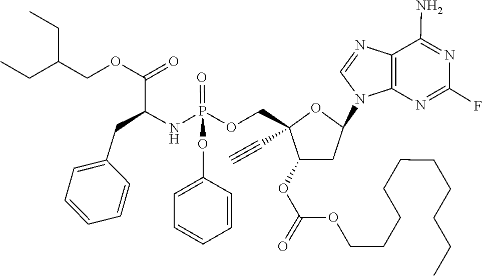 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCCCCC] | 2-Ethylbutyl ((S)-(((2R,3S,5R)-5-(6- amino-2-fluoro-9H-purin-9-yl)-3- (((decyloxy)carbonyl)oxy)-2- ethynyltetrahydrofuran-2- yl)methoxy)(phenoxy)phosphoryl)- L-phenylalaninate |
| 98 | 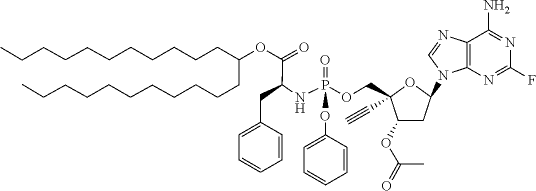 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(C)=O] | Tricosan-12-yl ((S)-(((2R,3S,5R)-3- acetoxy-5-(6-amino-2-fluoro-9H- purin-9-yl)-2- ethynyltetrahydrofuran-2- yl)methoxy)(phenoxy)phosphoryl)- L-phenylalaninate |
| 99 | 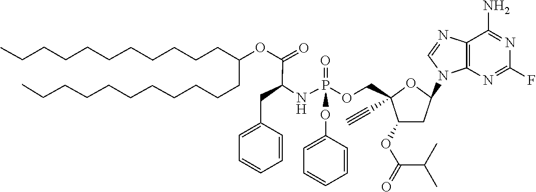 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)C(C)C] | Tricosan-12-yl ((S)-(((2R,3S,5R)-5- (6-amino-2-fluoro-9H-purin-9-yl)-2- ethynyl-3- (isobutyryloxy)tetrahydrofuran-2- yl)methoxy)(phenoxy)phosphoryl)- L-phenylalaninate |
| 100 | 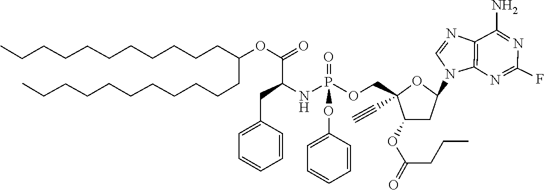 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)CCC] | (2R,3S,5R)-5-(6-Amino-2-fluoro- 9H-purin-9-yl)-2-ethynyl-2-((((S)- (((S)-1-oxo-3-phenyl-1-(tricosan- 12-yloxy)propan-2- yl)amino)(phenoxy)phosphoryl)oxy) methyl)tetrahydrofuran-3-yl butyrate |
| 101 | 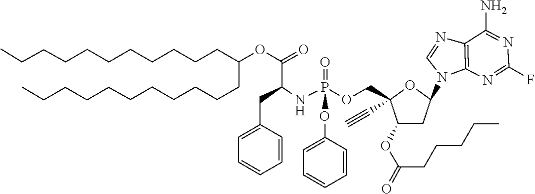 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)CCCCC] | (2R,3S,5R)-5-(6-Amino-2-fluoro- 9H-purin-9-yl)-2-ethynyl-2-((((S)- (((S)-1-oxo-3-phenyl-1-(tricosan- 12-yloxy)propan-2- yl)amino)(phenoxy)phosphoryl)oxy) methyl)tetrahydrofuran-3-yl hexanoate |
| 102 | 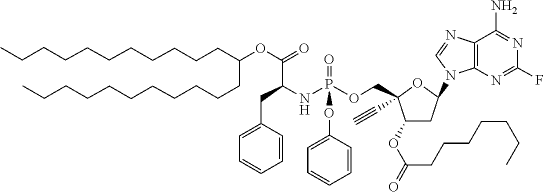 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)CCCCCCC] | (2R,3S,5R)-5-(6-Amino-2-fluoro- 9H-purin-9-yl)-2-ethynyl-2-((((S)- (((S)-1-oxo-3-phenyl-1-(tricosan- 12-yloxy)propan-2- yl)amino)(phenoxy)phosphoryl)oxy) methyl)tetrahydrofuran-3-yl octanoate |
| 103 | 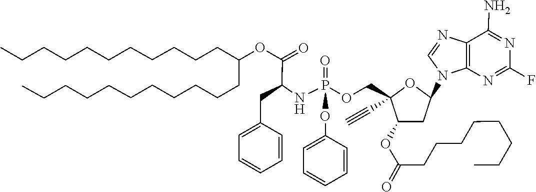 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-ethynyl-2-((((S)- (((S)-1-oxo-3-phenyl-1-(tricosan- 12-yloxy)propan-2- yl)amino)(phenoxy)phosphoryl)oxy) methyl)tetrahydrofuran-3-yl nonanoate |
| 104 | 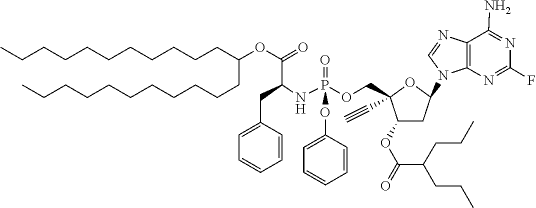 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-ethynyl-2-((((S)- (((S)-1-oxo-3-phenyl-1-(tricosan- 12-yloxy)propan-2- yl)amino)(phenoxy)phosphoryl)oxy) methyl)tetrahydrofuran-3-yl 2- propylpentanoate |
| 105 | 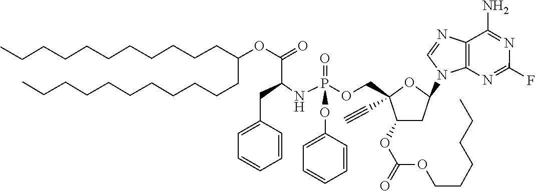 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)OCCCCCC] | Tricosan-12-yl ((S)-(((2R,3S,5R)-5- (6-amino-2-fluoro-9H-purin-9-yl)-2- ethynyl-3- (((hexyloxy)carbonyl)oxy)tetrahydro- furan-2- yl)methoxy)(phenoxy)phosphoryl)- L-phenylalaninate |
| 106 | 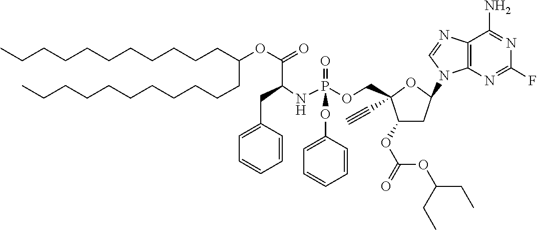 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)OC(CC)CC] | Tricosan-12-yl ((S)-(((2R,3S,5R)-5- (6-amino-2-fluoro-9H-purin-9-yl)-2- ethynyl-3-(((pentan-3- yloxy)carbonyl)oxy)tetrahydrofuran- 2- yl)methoxy)(phenoxy)phosphoryl)- L-phenylalaninate |
| 107 | 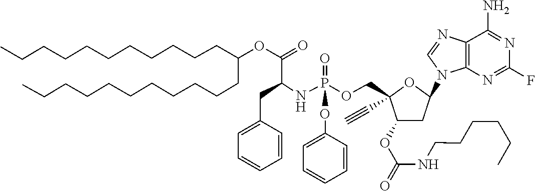 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)NCCCCCC] | Tricosan-12-yl ((S)-(((2R,3S,5R)-5- (6-amino-2-fluoro-9H-purin-9-yl)-2- ethynyl-3- ((hexylcarbamoyl)oxy)tetrahydrofuran- 2- yl)methoxy)(phenoxy)phosphoryl)- L-phenylalaninate |
| 108 | 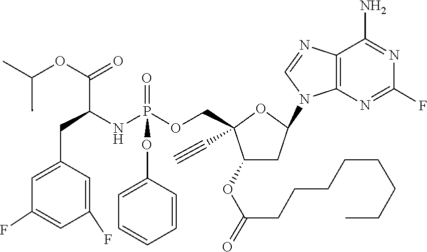 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-((((S)-(((S)-3-(3,5- difluorophenyl)- 1-isopropoxy-1- oxopropan-2- yl)amino)(phenoxy)phosphoryl)oxy) methyl)-2-ethynyltetrahydrofuran-3- yl nonanoate |
| 109 | 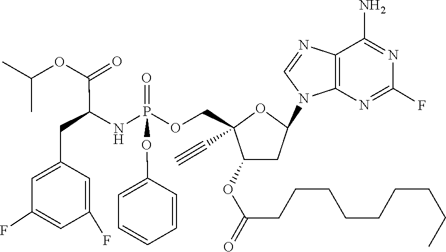 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((S)-(((S)-3-(3,5- difluorophenyl)-1-isopropoxy-1- oxopropan-2- yl)amino)(phenoxy)phosphoryl)oxy) methyl)-2-ethynyltetrahydrofuran-3- yl decanoate |
| 110 | 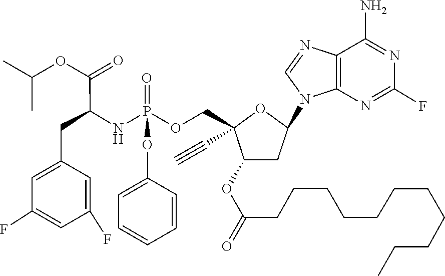 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-((((S)-(((S)-3-(3,5- difluorophenyl)-1-isopropoxy-1- oxopropan-2- yl)amino)(phenoxy)phosphoryl)oxy) methyl)-2-ethynyltetrahydrofuran-3- yl dodecanoate |
| 111 | 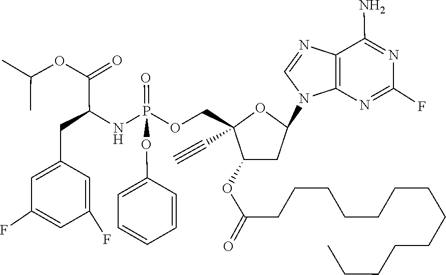 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-((((S)-(((S)-3-(3,5- difluorophenyl)-1-isopropoxy-1- oxopropan-2- yl)amino)(phenoxy)phosphoryl)oxy) methyl)-2-ethynyltetrahydrofuran-3- yl tetradecanoate |
| 112 | 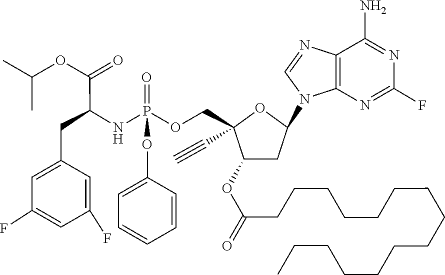 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-((((S)-(((S)-3-(3,5- difluorophenyl)-1-isopropoxy-1- oxopropan-2- yl)amino)(phenoxy)phosphoryl)oxy) methyl)-2-ethynyltetrahydrofuran-3- yl palmitate |
| 113 | 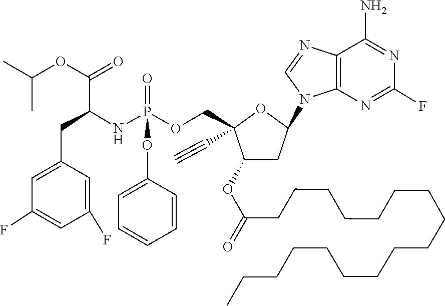 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCCCCCCCCCCC] | (2R,3S,5R)-5-(6-amino-2-fluoro- 9H-purin-9-yl)-2-((((S)-(((S)-3-(3,5- difluorophenyl)-1-isopropoxy-1- oxopropan-2- yl)amino)(phenoxy)phosphoryl)oxy) methyl)-2-ethynyltetrahydrofuran-3- yl stearate |
| 114 | 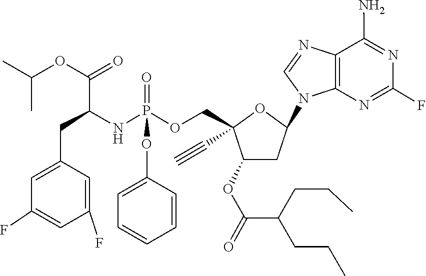 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-((((S)-(((S)-3-(3,5- difluorophenyl)-1-isopropoxy-1- oxopropan-2- yl)amino)(phenoxy)phosphoryl)oxy) methyl)-2-ethynyltetrahydrofuran-3- yl 2-propylpentanoate |
| 115 | 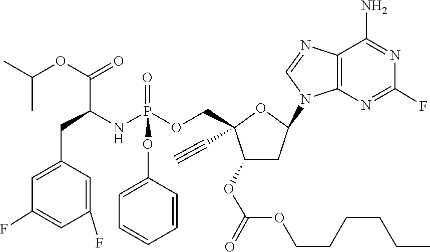 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OCCCCCC] | Isopropyl (S)-2-(((S)-(((2R,3S,5R)- 5-(6-amino-2-fluoro-9H-purin-9-yl)- 2-ethynyl-3- (((hexyloxy)carbonyl)oxy)tetrahydro- furan-2- yl)methoxy)(phenoxy)phosphoryl) amino)-3-(3,5- difluorophenyl)propanoate |
| 116 | 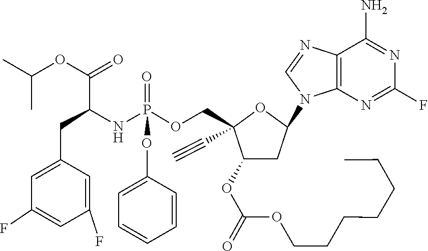 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OCCCCCCC] | Isopropyl (S)-2-(((S)-(((2R,3S,5R)- 5-(6-amino-2-fluoro-9H-purin-9-yl)- 2-ethynyl-3- (((heptyloxy)carbonyl)oxy)tetrahydro- furan-2- yl)methoxy)(phenoxy)phosphoryl) amino)-3-(3,5- difluorophenyl)propanoate |
| 117 | 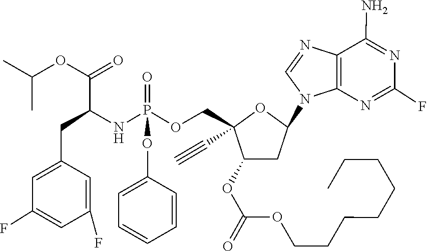 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OCCCCCCCC] | Isopropyl (S)-2-(((S)-(((2R,3S,5R)- 5-(6-amino-2-fluoro-9H-purin-9-yl)- 2-ethynyl-3- (((octyloxy)carbonyl)oxy)tetrahydro- furan-2- yl)methoxy)(phenoxy)phosphoryl) amino)-3-(3,5- difluorophenyl)propanoate |
| 118 | 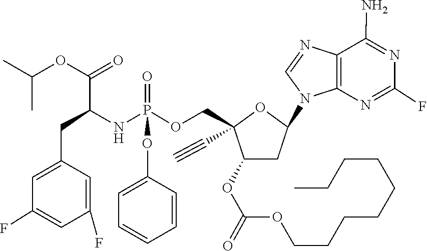 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OCCCCCCCCC] | Isopropyl (S)-2-(((S)-(((2R,3S,5R)- 5-(6-amino-2-fluoro-9H-purin-9-yl)- 2-ethynyl-3- (((nonyloxy)carbonyl)oxy)tetrahydro- furan-2- yl)methoxy)(phenoxy)phosphoryl) amino)-3-(3,5- difluorophenyl)propanoate |
| 119 | 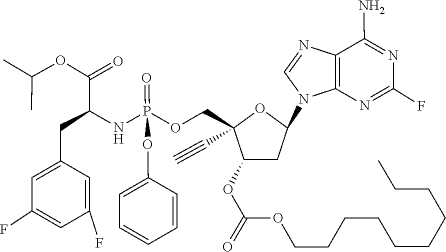 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OCCCCCCCCCC] | Isopropyl (S)-2-(((S)-(((2R,3S,5R)- 5-(6-amino-2-fluoro-9H-purin-9-yl)- 3-(((decyloxy)carbonyl)oxy)-2- ethynyltetrahydrofuran-2- yl)methoxy)(phenoxy)phosphoryl) amino)-3-(3,5- difluorophenyl)propanoate |
| 120 | 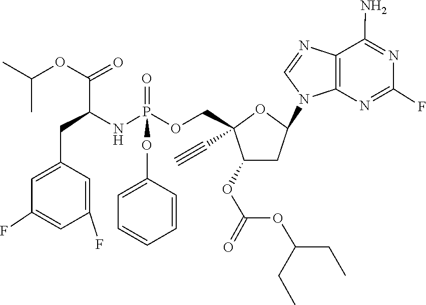 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OC(CC)CC] | Isopropyl (S)-2-(((S)-(((2R,3S,5R)- 5-(6-amino-2-fluoro-9H-purin-9-yl)- 2-ethynyl-3-(((pentan-3- yloxy)carbonyl)oxy)tetrahydrofuran- 2- yl)methoxy)(phenoxy)phosphoryl) amino)-3-(3,5- difluorophenyl)propanoate |
| 121 | 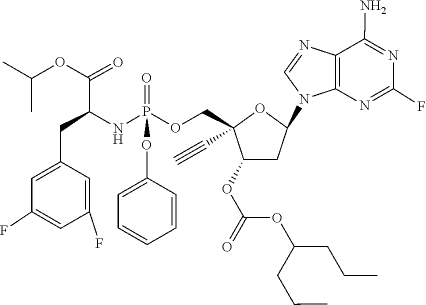 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OC(CCC)CCC] | Isopropyl (S)-2-(((S)-(((2R,3S,5R)- 5-(6-amino-2-fluoro-9H-purin-9-yl)- 2-ethynyl-3-(((heptan-4- yloxy)carbonyl)oxy)tetrahydrofuran- 2- yl)methoxy)(phenoxy)phosphoryl) amino)-3-(3,5- difluorophenyl)propanoate |
| 122 | 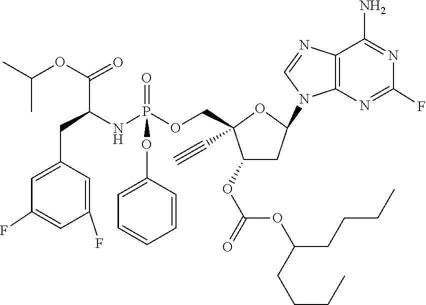 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OC(CCCC)CCCC] | Isopropyl (S)-2-(((S)-(((2R,3S,5R)- 5-(6-amino-2-fluoro-9H-purin-9-yl)- 2-ethynyl-3-(((nonan-5- yloxy)carbonyl)oxy)tetrahydrofuran- 2- yl)methoxy)(phenoxy)phosphoryl) amino)-3-(3,5- difluorophenyl)propanoate |
| 123 | 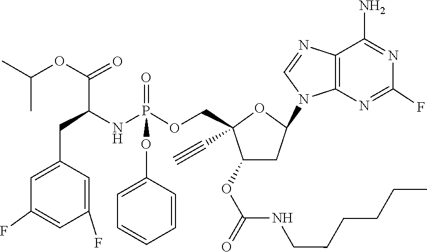 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)NCCCCCC] | Isopropyl (S)-2-(((S)-(((2R,3S,5R)- 5-(6-amino-2-fluoro-9H-purin-9-yl)- 2-ethynyl-3- ((hexylcarbamoyl)oxy)tetrahydrofuran- 2- yl)methoxy)(phenoxy)phosphoryl) amino)-3-(3,5- difluorophenyl)propanoate |
| 124 | 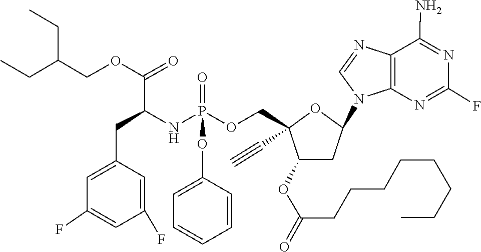 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-((((S)-(((S)-3-(3,5- difluorophenyl)-1-(2-ethylbutoxy)-1- oxopropan-2- yl)amino)(phenoxy)phosphoryl)oxy) methyl)-2-ethynyltetrahydrofuran-3- yl nonanoate |
| 125 | 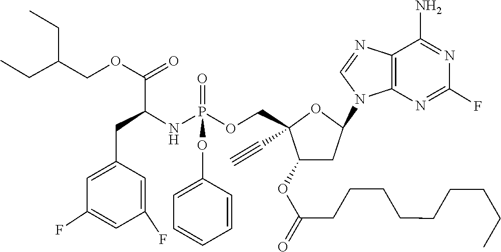 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((S)-(((S)-3-(3,5- difluorophenyl)-1-(2-ethylbutoxy)-1- oxopropan-2- yl)amino)(phenoxy)phosphoryl)oxy) methyl)-2-ethynyltetrahydrofuran-3- yl decanoate |
| 126 | 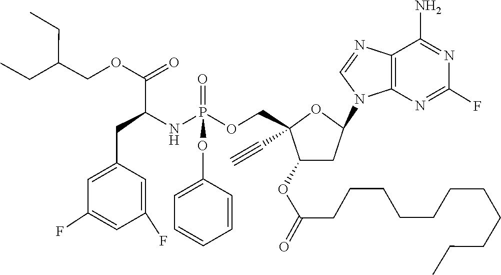 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-((((S)-(((S)-3-(3,5- difluorophenyl)-1-(2-ethylbutoxy)-1- oxopropan-2- yl)amino)(phenoxy)phosphoryl)oxy) methyl)-2-ethynyltetrahydrofuran-3- yl dodecanoate |
| 127 | 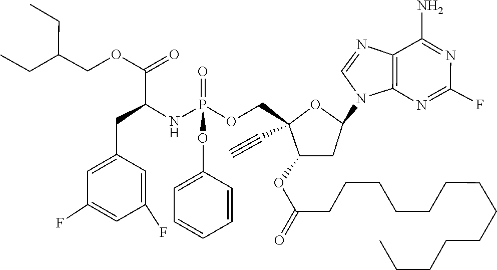 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-((((S)-(((S)-3-(3,5- difluorophenyl)-1-(2-ethylbutoxy)-1- oxopropan-2- yl)amino)(phenoxy)phosphoryl)oxy) methyl)-2-ethynyltetrahydrofuran-3- yl tetradecanoate |
| 128 | 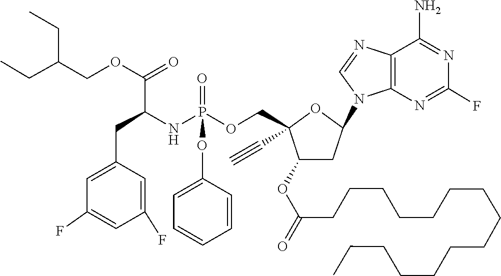 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-((((S)-(((S)-3-(3,5- difluorophenyl)-1-(2-ethylbutoxy)-1- oxopropan-2- yl)amino)(phenoxy)phosphoryl)oxy) methyl)-2-ethynyltetrahydrofuran-3- yl palmitate |
| 129 | 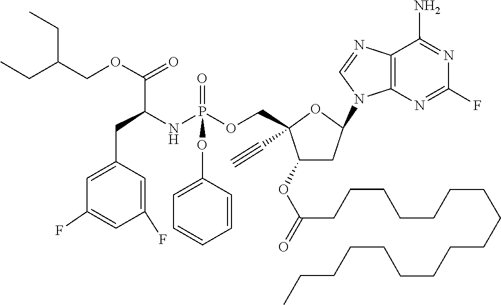 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCCCCCCCC] | (2R,3S,5R)-5-(6-Amino-2-fluoro- 9H-purin-9-yl)-2-((((S)-(((S)-3-(3,5- difluorophenyl)-1-(2-ethylbutoxy)-1- oxopropan-2- yl)amino)(phenoxy)phosphoryl)oxy) methyl)-2-ethynyltetrahydrofuran-3- yl stearate |
| 130 | 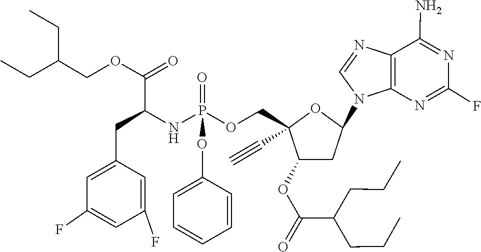 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-((((S)-(((S)-3-(3,5- difluorophenyl)-1-(2-ethylbutoxy)-1- oxopropan-2- yl)amino)(phenoxy)phosphoryl)oxy) methyl)-2-ethynyltetrahydrofuran-3- yl 2-propylpentanoate |
| 131 | 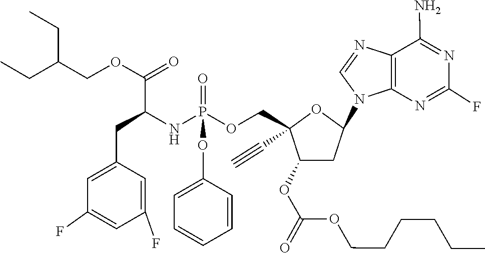 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OCCCCCC] | 2-Ethylbutyl (S)-2-(((S)- (((2R,3S,5R)-5-(6-amino-2-fluoro- 9H-purin-9-yl)-2-ethynyl-3- (((hexyloxy)carbonyl)oxy)tetrahydro- furan-2- yl)methoxy)(phenoxy)phosphoryl) amino)-3-(3,5- difluorophenyl)propanoate |
| 132 | 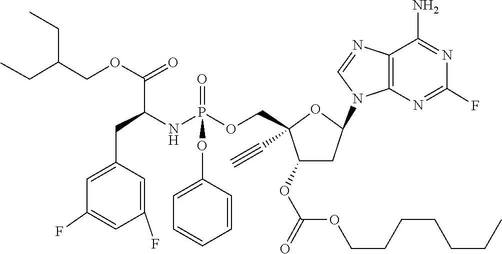 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OCCCCCCC] | 2-Ethylbutyl (S)-2-(((S)- (((2R,3S,5R)-5-(6-amino-2-fluoro- 9H-purin-9-yl)-2-ethynyl-3- (((heptyloxy)carbonyl)oxy)tetrahydro- furan-2- yl)methoxy)(phenoxy)phosphoryl) amino)-3-(3,5- difluorophenyl)propanoate |
| 133 | 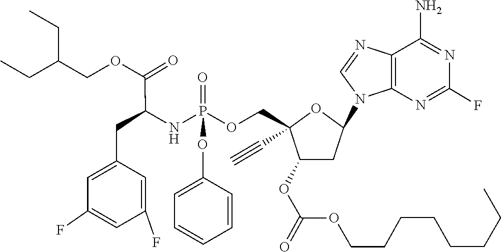 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OCCCCCCCC] | 2-Ethylbutyl (S)-2-(((S)- (((2R,3S,5R)-5-(6-amino-2-fluoro- 9H-purin-9-yl)-2-ethynyl-3- (((octyloxy)carbonyl)oxy)tetrahydro- furan-2- yl)methoxy)(phenoxy)phosphoryl) amino)-3-(3,5- difluorophenyl)propanoate |
| 134 | 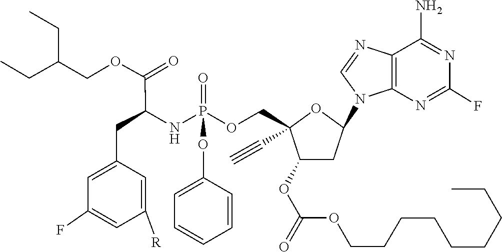 | 2-Ethylbutyl (S)-2-(((S)- (((2R,3S,5R)-5-(6-amino-2-fluoro- 9H-purin-9-yl)-2-ethynyl-3- (((nonyloxy)carbonyl)oxy)tetrahydro- furan-2- yl)methoxy)(phenoxy)phosphoryl) amino)-3-(3,5- difluorophenyl)propanoate |
| 135 | 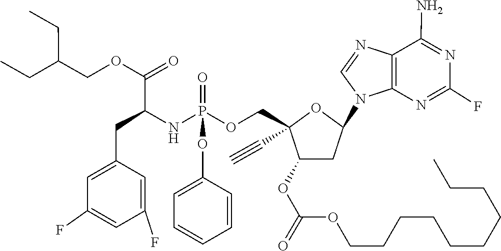 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OCCCCCCCCCC] | 2-Ethylbutyl (S)-2-(((S)- (((2R,3S,5R)-5-(6-amino-2-fluoro- 9H-purin-9-yl)-3- (((decyloxy)carbonyl)oxy)-2- ethynyltetrahydrofuran-2- yl)methoxy)(phenoxy)phosphoryl) amino)-3-(3,5- difluorophenyl)propanoate |
| 136 | 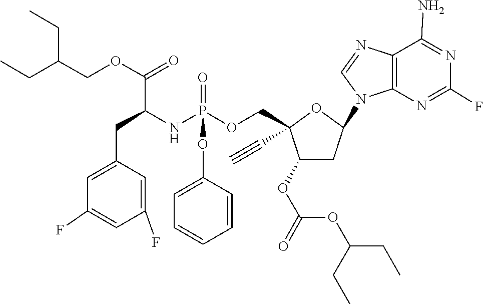 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OC(CC)CC] | 2-Ethylbutyl (S)-2-(((S)- (((2R,3S,5R)-5-(6-amino-2-fluoro- 9H-purin-9-yl)-2-ethynyl-3- (((pentan-3- yloxy)carbonyl)oxy)tetrahydrofuran- 2- yl)methoxy)(phenoxy)phosphoryl) amino)-3-(3,5- difluorophenyl)propanoate |
| 137 | 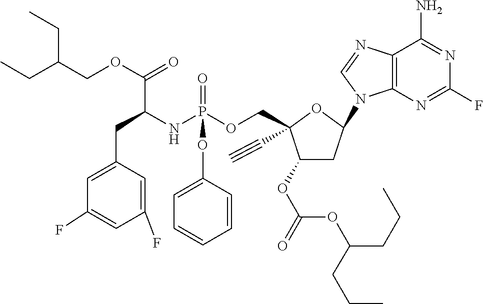 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OC(CCC)CCC] | 2-Ethylbutyl (S)-2-(((S)- (((2R,3S,5R)-5-(6-amino-2-fluoro- 9H-purin-9-yl)-2-ethynyl-3- (((heptan-4- yloxy)carbonyl)oxy)tetrahydrofuran- 2- yl)methoxy)(phenoxy)phosphoryl) amino)-3-(3,5- difluorophenyl)propanoate |
| 138 | 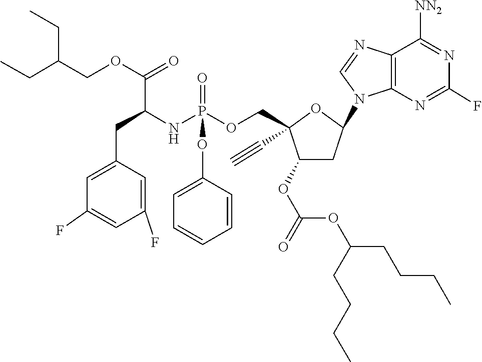 | 2-Ethylbutyl (S)-2-(((S)- (((2R,3S,5R)-5-(6-amino-2-fluoro- 9H-purin-9-yl)-2-ethynyl-3- (((nonan-5- yloxy)carbonyl)oxy)tetrahydrofuran- 2- yl)methoxy)(phenoxy)phosphoryl) amino)-3-(3,5- difluorophenyl)propanoate |
| 139 | 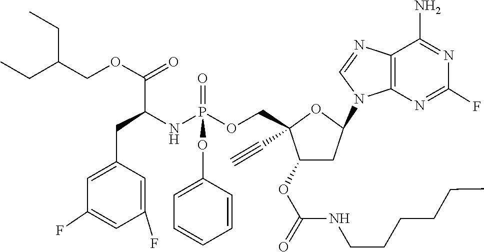 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)NCCCCCC] | 2-Ethylbutyl (S)-2-(((S)- (((2R,3S,5R)-5-(6-amino-2-fluoro- 9H-purin-9-yl)-2-ethynyl-3- ((hexylcarbamoyl)oxy)tetrahydrofuran- 2- yl)methoxy)(phenoxy)phosphoryl) amino)-3-(3,5- difluorophenyl)propanoate |

and pharmaceutically acceptable salts thereof.

In one embodiment, the present invention encompasses each individual compound listed in the above Table 1, or a pharmaceutically acceptable salt thereof.

In various embodiments, prodrugs of any of the compounds of formula (I) set forth herein are also within the scope of the present invention.

In accordance with one embodiment of the present invention, there is provided a pharmaceutical composition comprising a compound of Formulas (I) or a pharmaceutically acceptable salt thereof, and a pharmaceutically acceptable excipient. In a further embodiment, the compound is present in amorphous form. In a further embodiment, the compound is present in crystalline form. In a further embodiment, the pharmaceutical composition is in a tablet form. In a further embodiment, the pharmaceutical composition is in parenteral form. In a further embodiment, the compound is present as a spray dried dispersion.

In accordance with one embodiment of the present invention, there is provided a method of treating an HIV infection in a subject comprising administering to the subject a compound of Formulas (I) or a pharmaceutically acceptable salt thereof.

In accordance with one embodiment of the present invention, there is provided a method of treating an HIV infection in a subject comprising administering to the subject a pharmaceutical composition as described herein.

In accordance with one embodiment of the present invention, there is provided a method of preventing an HIV infection in a subject at risk for developing an HIV infection, comprising administering to the subject a compound of Formulas (I) or a pharmaceutically acceptable salt thereof.

In accordance with one embodiment of the present invention, there is provided the use of a compound of Formula (I) in the manufacture of a medicament for treating an HIV infection.

In accordance with one embodiment of the present invention, there is provided the use of a compound of Formula (I) in the manufacture of a medicament for preventing an HIV infection.

In accordance with one embodiment of the present invention, there is provided a compound according to Formula (I) for use in treating an HIV infection.

In accordance with one embodiment of the present invention, there is provided a compound according to Formula (I) for use in preventing an HIV infection.

In accordance with one embodiment of the present invention, there is provided a method of preventing an HIV infection in a subject at risk for developing an HIV infection, comprising administering to the subject a pharmaceutical composition as described herein.

Furthermore, the compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and I isomers can be prepared using chiral synthons or chiral reagents, or resolved using conventional techniques. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

In another embodiment of the invention, there is provided a compound of Formula (I) wherein the compound or salt of the compound is used in the manufacture of a medicament for use in the treatment of an HIV infection in a human.

In another embodiment of the invention, there is provided a compound of Formula (I) wherein the compound or salt of the compound is used in the manufacture of a medicament for use in the prevention of an HIV infection in a human.

In one embodiment, the pharmaceutical formulation containing a compound of Formula (I) or a salt thereof is a formulation adapted for parenteral administration. In another embodiment, the formulation is a long-acting parenteral formulation. In a further embodiment, the formulation is a nano-particle formulation.

The compounds of the present invention and their salts, solvates, or other pharmaceutically acceptable derivatives thereof, may be employed alone or in combination with other therapeutic agents. Therefore, in other embodiments, the methods of treating and/or preventing an HIV infection in a subject may in addition to administration of a compound of Formula (I) further comprise administration of one or more additional pharmaceutical agents active against HIV.

In such embodiments, the one or more additional agents active against HIV is selected from the group consisting of zidovudine, didanosine, lamivudine, zalcitabine, abacavir, stavudine, adefovir, adefovir dipivoxil, fozivudine, todoxil, emtricitabine, alovudine, amdoxovir, elvucitabine, nevirapine, delavirdine, efavirenz, loviride, immunocal, oltipraz, capravirine, lersivirine, GSK2248761, TMC-278, TMC-125, etravirine, saquinavir, ritonavir, indinavir, nelfinavir, amprenavir, fosamprenavir, brecanavir, darunavir, atazanavir, tipranavir, palinavir, lasinavir, enfuvirtide, T-20, T-1249, PRO-542, PRO-140, TNX-355, BMS-806, BMS-663068 and BMS-626529, 5-Helix, raltegravir, elvitegravir, dolutegravir, cabotegravir, bictegravir, vicriviroc (Sch-C), Sch-D, TAK779, maraviroc, TAK449, didanosine, tenofovir, lopinavir, and darunavir.

As such, the compounds of the present invention of Formulas (I) and any other pharmaceutically active agent(s) may be administered together or separately and, when administered separately, administration may occur simultaneously or sequentially, in any order. The amounts of the compounds of Formula (I) of the present invention and the other pharmaceutically active agent(s) and the relative timings of administration will be selected in order to achieve the desired combined therapeutic effect. The administration in combination of a compound of the present invention of Formula (I) and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The amounts of the compound(s) of Formula (I) or salts thereof and the other pharmaceutically active agent(s) and the relative timings of administration will be selected in order to achieve the desired combined therapeutic effect.

In addition, the compounds of the present invention of Formula (I) may be used in combination with one or more other agents that may be useful in the prevention or treatment of HIV. Examples of such agents include:

Nucleotide reverse transcriptase inhibitors such as zidovudine, didanosine, lamivudine, zalcitabine, abacavir, stavudine, adefovir, adefovir dipivoxil, fozivudine, todoxil, emtricitabine, alovudine, amdoxovir, elvucitabine, and similar agents;
Non-nucleotide reverse transcriptase inhibitors (including an agent having anti-oxidation activity such as immunocal, oltipraz, etc.) such as nevirapine, delavirdine, efavirenz, loviride, immunocal, oltipraz, capravirine, lersivirine, doravirine, GSK2248761, TMC-278, TMC-125, etravirine, and similar agents;
Protease inhibitors such as saquinavir, ritonavir, indinavir, nelfinavir, amprenavir, fosamprenavir, brecanavir, darunavir, atazanavir, tipranavir, palinavir, lasinavir, and similar agents;
Entry, attachment and fusion inhibitors such as enfuvirtide (T-20), T-1249, PRO-542, PRO-140, TNX-355, BMS-806, BMS-663068 (Fostemsavir), BMS-626529 (Temsavir), 5-Helix and similar agents;
Integrase inhibitors such as raltegravir, elvitegravir, dolutegravir, bictegravir, cabotegravir and similar agents;
Maturation inhibitors such as PA-344 and PA-457, and similar agents; and
CXCR4 and/or CCR5 inhibitors such as vicriviroc (Sch-C), Sch-D, TAK779, maraviroc (UK 427,857), TAK449, as well as those disclosed in WO 02/74769, PCT/US03/39644, PCT/US03/39975, PCT/US03/39619, PCT/US03/39618, PCT/US03/39740, and PCT/US03/39732, and similar agents.
CAPSID inhibitors such GS-6207, and similar agents.

Further examples where the compounds of the present invention may be used in combination with one or more agents useful in the prevention or treatment of HIV are found in Table 2.

TABLE 2

| | Brand | | |
|---|---|---|---|
| FDA Approval | Name | Generic Name | Manufacturer |
| Nucleoside | | | |
| Reverse | | | |
| Transcriptase | | | |
| Inhibitors | | | |
| (NRTIs) | | | |
| 1987 | Retrovir | zidovudine, | GlaxoSmithKline |
| | | azidothymidine, | |
| | | AZT, ZDV | |
| 1991 | Videx | didanosine, | Bristol-Myers |
| | | dideoxyinosine, | Squibb |
| | | ddI | |
| 1992 | Hivid | zalcitabine, | Roche |
| | | dideoxycytidine, | Pharmaceuticals |
| | | ddC | |
| 1994 | Zerit | stavudine, d4T | Bristol-Myers |
| | | | Squibb |
| 1995 | Epivir | lamivudine, 3TC | GlaxoSmithKline |
| 1997 | Combivir | lamivudine + | GlaxoSmithKline |
| | | zidovudine | |
| 1998 | Ziagen | abacavir sulfate, | GlaxoSmithKline |
| | | ABC | |
| 2000 | Trizivir | abacavir + | GlaxoSmithKline |
| | | lamivudine + | |
| | | zidovudine | |
| 2000 | Videx EC | enteric coated | Bristol-Myers |
| | | didanosine, ddI | Squibb |
| | | EC | |
| 2001 | Viread | tenofovir disoproxil | Gilead Sciences |
| | | fumarate, TDF | |
| 2003 | Emtriva | emtricitabine, FTC | Gilead Sciences |
| 2004 | Epzicom | abacavir + | GlaxoSmithKline |
| | | lamivudine | |
| 2004 | Truvada | emtricitabine + | Gilead Sciences |
| | | tenofovir disoproxil | |
| | | fumarate | |
| Non-Nucleosides | | | |
| Reverse | | | |
| Transcriptase | | | |
| Inhibitors | | | |
| (NNRTIs) | | | |
| 1996 | Viramune | nevirapine, NVP | Boehringer |
| | | | Ingelheim |
| 1997 | Rescriptor | delavirdine, DLV | Pfizer |
| 1998 | Sustiva | efavirenz, EFV | Bristol-Myers |
| | | | Squibb |
| 2008 | Intelence | Etravirine | Tibotec |
| | | | Therapeutics |
| 2011 | Edurant | Rilpivirine | Tibotec |
| | | | Therapeutics |
| 2018 | Pifeltro | Doravirine | Merck |
| Protease | | | |
| Inhibitors (PIs) | | | |
| 1995 | Invirase | saquinavir | Roche |
| | | mesylate, SQV | Pharmaceuticals |
| 1996 | Norvir | ritonavir, RTV | Abbott |
| | | | Laboratories |
| 1996 | Crixivan | indinavir, IDV | Merck |
| 1997 | Viracept | nelfinavir | Pfizer |
| | | mesylate, NFV | |
| 1997 | Fortovase | saquinavir (no | Roche |
| | | longer marketed) | Pharmaceuticals |
| 1999 | Agenerase | amprenavir, APV | GlaxoSmithKline |
| 2000 | Kaletra | lopinavir + | Abbott |
| | | ritonavir, LPV/RTV | Laboratories |
| 2003 | Reyataz | atazanavir sulfate, | Bristol-Myers |
| | | ATV | Squibb |
| 2003 | Lexiva | fosamprenavir | GlaxoSmithKline |
| | | calcium, FOS-APV | |
| 2005 | Aptivus | tripranavir, TPV | Boehringer |
| | | | Ingelheim |
| 2006 | Prezista | Darunavir | Tibotec |
| | | | Therapeutics |
| Fusion Inhibitors | | | |
| 2003 | Fuzeon | Enfuvirtide, T-20 | Roche |
| | | | Pharmaceuticals & |
| | | | Trimeris |
| Entry Inhibitors | | | |
| 2007 | Selzentry | Maraviroc | Pfizer |
| Integrase | | | |
| Inhibitors | | | |
| 2007 | Isentress | Raltegravir | Merck |
| 2013 | Tivicay | Dolutegravir | ViiV Healthcare |
| 2018 | | Bictegravir | Gilead Sciences |
| — | — | Cabotegravir | ViiV Healthcare |
| Capsid Inhibitors | | | |
| — | | GS-6207 | Gilead Sciences |

The scope of combinations of compounds of this invention with HIV agents is not limited to those mentioned above, but includes in principle any combination with any pharmaceutical composition useful for the treatment and/or prevention of HIV. As noted, in such combinations the compounds of the present invention and other HIV agents may be administered separately or in conjunction. In addition, one agent may be prior to, concurrent to, or subsequent to the administration of other agent(s).

The present invention may be used in combination with one or more agents useful as pharmacological enhancers as well as with or without additional compounds for the prevention or treatment of HIV. Examples of such pharmacological enhancers (or pharmakinetic boosters) include, but are not limited to, ritonavir, GS-9350, and SPI-452. Ritonavir is 10-hydroxy-2-methyl-5-(1-methyethyl)-1-1[2-(1-methylethyl)-4-thiazolyl]-3,6-dioxo-8,11-bis(phenylmethyl)-2,4,7,12-tetraazatridecan-13-oic acid, 5-thiazolylmethyl ester, [5S-(5S*,8R*,10R*,11R*)] and is available from Abbott Laboratories of Abbott park, Illinois, as Norvir. Ritonavir is an HIV protease inhibitor indicated with other antiretroviral agents for the treatment of HIV infection. Ritonavir also inhibits P450 mediated drug metabolism as well as the P-gycoprotein (Pgp) cell transport system, thereby resulting in increased concentrations of active compound within the organism.

GS-9350 is a compound being developed by Gilead Sciences of Foster City California as a pharmacological enhancer.
SPI-452 is a compound being developed by Sequoia Pharmaceuticals of Gaithersburg, Maryland, as a pharmacological enhancer.

In one embodiment of the present invention, a compound of Formula (I) is used in combination with ritonavir. In one embodiment, the combination is an oral fixed dose combination. In another embodiment, the compound of Formula (I) is formulated as a long acting parenteral injection and ritonavir is formulated as an oral composition. In one embodiment, a kit containing the compound of Formula (I) is formulated as a long acting parenteral injection and ritonavir formulated as an oral composition. In another embodiment, the compound of Formula (I) is formulated as a long acting parenteral injection and ritonavir is formulated as an injectable composition. In one embodiment, a kit containing the compound of Formula (I) is formulated as a long acting parenteral injection and ritonavir formulated as an injectable composition.

In another embodiment of the present invention, a compound of Formula (I) is used in combination with GS-9350. In one embodiment, the combination is an oral fixed dose combination. In another embodiment, the compound of Formula (I) is formulated as a long acting parenteral injection and GS-9350 is formulated as an oral composition. In one embodiment, there is provided a kit containing the compound of Formula (I) is formulated as a long acting parenteral injection and GS-9350 formulated as an oral composition. In another embodiment, the compound of Formula (I) is formulated as a long acting parenteral injection and GS-9350 is formulated as an injectable composition. In one embodiment, is a kit containing the compound of Formula (I) is formulated as a long acting parenteral injection and GS-9350 formulated as an injectable composition.

In one embodiment of the present invention, a compound of Formula (I) is used in combination with SPI-452. In one embodiment, the combination is an oral fixed dose combination. In another embodiment, the compound of Formula (I) is formulated as a long acting parenteral injection and SPI-452 is formulated as an oral composition. In one embodiment, there is provided a kit containing the compound of Formula (I) formulated as a long acting parenteral injection and SPI-452 formulated as an oral composition. In another embodiment, the compound of Formula (I) is formulated as a long acting parenteral injection and SPI-452 is formulated as an injectable composition. In one embodiment, there is provided a kit containing the compound of Formula (I) formulated as a long acting parenteral injection and SPI-452 formulated as an injectable composition.

In one embodiment of the present invention, a compound of Formula (I) is used in combination with compounds which are found in previously filed PCT/CN2011/0013021, which is herein incorporated by reference.

The above other therapeutic agents, when employed in combination with the chemical entities described herein, may be used, for example, in those amounts indicated in the Physicians' Desk Reference (PDR) or as otherwise determined by one of ordinary skill in the art.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I).

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I), wherein said virus is an HIV virus. In some embodiments, the HIV virus is the HIV-1 virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I) further comprising administration of a therapeutically effective amount of one or more agents active against an HIV virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I), further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CAPSID inhibitors, CXCR4 inhibitors; and CCR5 inhibitors.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I).

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I), wherein said virus is an HIV virus. In some embodiments, the HIV virus is the HIV-1 virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I), further comprising administration of a therapeutically effective amount of one or more agents active against an HIV virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I), further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CAPSID inhibitors, CXCR4 inhibitors; and CCR5 inhibitors.

In further embodiments, the compound of the present invention of Formula (I) or a pharmaceutically acceptable salt thereof, is selected from the group of compounds set forth in Table 1 above.

The compounds of Table 1 were synthesized according to the Synthetic Methods, General Schemes, and the Examples described below.

In another embodiment, there is provided a pharmaceutical composition comprising a pharmaceutically acceptable diluent and a therapeutically effective amount of a compound of Formula (I) or a pharmaceutically acceptable salt thereof.

In certain embodiments, the compound(s) of the present invention, or a pharmaceutically acceptable salt thereof, is chosen from the compounds set forth in Table 1.

The compounds of Formula (I) of the invention may exist in both unsolvated and solvated forms. The term ‘solvate’ comprises the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO.

Compounds of Formula (I) containing one or more asymmetric carbon atoms can exist as two or more stereoisomers. Where a compound of Formula (I) contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds present invention are all stereoisomers, geometric isomers and tautomeric forms of the compounds of Formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC), Supercritical fluid chromatography (SFC).

Alternatively, the racemate (or a racemic precursor) may be reacted with a suitable optically active compound, for example, an alcohol, or, in the case where the compound of Formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC or SFC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of Formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labelled compounds of Formula (I) can generally be prepared by conventional techniques known to those skilled in the art or by processes analogous to those described herein using an appropriate isotopically-labelled reagents in place of the non-labelled reagent previously employed.

The compounds of the present invention may be administered as prodrugs. Thus, certain derivatives of compounds of Formula (I), which may have little or no pharmacological activity themselves can, when administered into or onto the body, be converted into compounds of Formula (I) as ‘prodrugs’. One example of a compound that such prodrugs may encompass is 4′-ethylnyl-2-fluoro-2′-dooxyadenosine (EFdA) disclosed e.g., in U.S. Pat. No. 7,339,053. The compounds of the present invention may be administered as prodrugs. In one embodiment, the compounds of the invention are prodrugs of 4′-ethynyl-2-fluoro-2′-deoxyadenosine (EFdA) disclosed e.g., in U.S. Pat. No. 7,339,053, which is a nucleoside reverse transcriptase inhibitor of the formula:

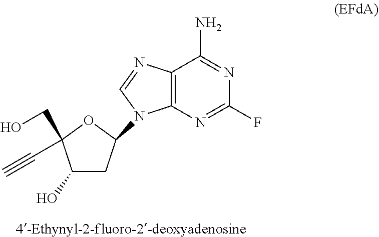 [SMILES: C#C[C@]1(CO)O[C@@H](n2cnc3c(N)nc(F)nc32)C[C@@H]1O]

The prodrugs are useful in that they are believed to be capable of modulating physicochemical properties, facilitating multiple dosing paradigms and improving pharmacokinetic and/or pharmacodynamic profiles of the active parent (EfdA). For example, the prodrugs may facilitate long-acting parenteral dosing modalities, and/or improvements in antiviral persistence profiles as compared to EFdA.

Administration of the chemical entities and combinations of entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used. Examples of dosing include, without limitation, once every seven days for oral, once every eight weeks for intramuscular, or once every six months for subcutaneous.

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pennsylvania.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition may comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used the route and form of administration, and other factors. The drug can be administered more than once a day, such as once or twice a day.

In general, the chemical entities will be administered as pharmaceutical compositions by any one of the following routes: oral, systemic (e.g., transdermal, intranasal or by suppository), or parenteral (e.g., intramuscular, intravenous or subcutaneous) administration. In certain embodiments, oral administration with a convenient daily dosage regimen that can be adjusted according to the degree of affliction may be used. Compositions can take the form of tablets, pills, capsules, semisolids, powders, sustained release formulations, solutions, suspensions, elixirs, aerosols, or any other appropriate compositions. Another manner for administering the provided chemical entities is inhalation.

The choice of formulation depends on various factors such as the mode of drug administration and bioavailability of the drug substance. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDIs typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, at least one chemical entity described herein in combination with at least one pharmaceutically acceptable excipient. Acceptable excipients are non-toxic, aid administration, and do not adversely affect the therapeutic benefit of the at least one chemical entity described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Compressed gases may be used to disperse a chemical entity described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the chemical entity in a composition can vary within the full range employed by those skilled in the art. Typically, the composition will contain, on a weight percent (wt %) basis, from about 0.01-99.99 wt % of at least one chemical entity described herein based on the total composition, with the balance being one or more suitable pharmaceutical excipients. In certain embodiments, the at least one chemical entity described herein is present at a level of about 1-80 wt %.

In various embodiments, pharmaceutical compositions of the present invention encompass compounds of Formula (I), salts thereof, and combinations of the above.

### Synthetic Methods

The methods of synthesis may employ readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, the methods of this invention may employ protecting groups which prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers or diastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The compounds of Formula (I) herein including those in Examples 1-16 contain a phosphorus chiral center. The isomer mixture in each of Examples 1-16, were separated, providing an Isomer #A e.g. Isomer 1A (faster eluting isomer) and an Isomer #B, e g. Isomer 1B (slower eluting isomer), based on their observed elution order resulting from the separation as performed in the Example. Where retention times are shown, they are provided only to show, the relative order of elution of each isomer in an Example. Elution order of separated isomers may differ if performed under conditions different than those employed herein. Absolute stereochemistry (R or S) of the phosphorus chiral center in each of the “A” and “B” stereoisomers in Examples 1 to 16 was not determined. An asterisk (*) may be used in the associated chemical structure drawings of the Example compounds to indicate the phosphorus chiral center.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Ernka-Chemce or Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

Unless specified to the contrary, the reactions described herein may take place at atmospheric pressure, generally within a temperature range from −78° C. to 200° C. Further, except as employed in the Example or as otherwise specified, reaction times and conditions are intended to be approximate, e.g., taking place at about atmospheric pressure within a temperature range of about −78° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith, including, for example, benzene, toluene, acetonitrile, tetrahydrofuranyl (“THF”), dimethylformamide (“DMF”), chloroform, methylene chloride (or dichloromethane or DCM), diethyl ether, methanol, N-methylpyrrolidone (“NMP”), pyridine and the like.

Isolation and purification of the chemical entities and intermediates described herein can be affected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

### EXAMPLE AND GENERAL SYNTHESIS

The following example and prophetic synthesis method serve to more fully describe the manner of making and using the above-described invention. It is understood that this in no way serve to limit the true scope of the invention, but rather is presented for illustrative purposes. Unless otherwise specified, the following abbreviations have the following meanings. If an abbreviation is not defined, it has its generally accepted meaning.
aq.=aqueous
μL=microliters
μM=micromolar
NMR=nuclear magnetic resonance
Boc=tert-butoxycarbonyl
br=broad
Cbz=benzyloxycarbonyl
d=doublet
° C.=degrees Celsius
DCM=dichloromethane
dd=doublet of doublets
DIPEA=N,N-diisopropylethylamine
DMAP=N,N-dimethylaminopyridine
DMF=N,N-dimethylformamide
DMSO=dimethylsufoxide
EDC=N-(3-Dimethylaminopropyl)-N′-ethylcarbodiimide hydrochloride
EtOAc=ethyl acetate
g=gram
h or hr=hour(s)
HPLC=high performance liquid chromatography
Hz=Hertz
IU=International Units
IC₅₀=50% inhibitory concentration
J=coupling constant in Hz
LCMS=liquid chromatography mass spectrometry
m=multiplet
M=molar concentration
M+H=parent mass spectrum peak plus H
mg=milligram
min=minute(s)
mL=milliliter
mM=millimolar
mm=millimeters
mmol=millimole
MMTr=monomethoxytrityl
MS=mass spectrum
MTBE=methyl tert-butyl ether
nM=nanomolar
PE=petroleum ether
ppm=parts per million
q.s.=sufficient amount
s=singlet
RT=retention time
sat.=saturated
t=triplet
TBDMS=tert-butyldimethylsilyl
TBDPS=tert-butyldiphenylsilyl
TEA=triethylamine
THF=tetrahydrofuran
TMS=trimethylsilyl

Additionally, various compounds of the invention may be made, in one embodiment, by way of the general synthesis routes set forth in Schemes 1-2 below:

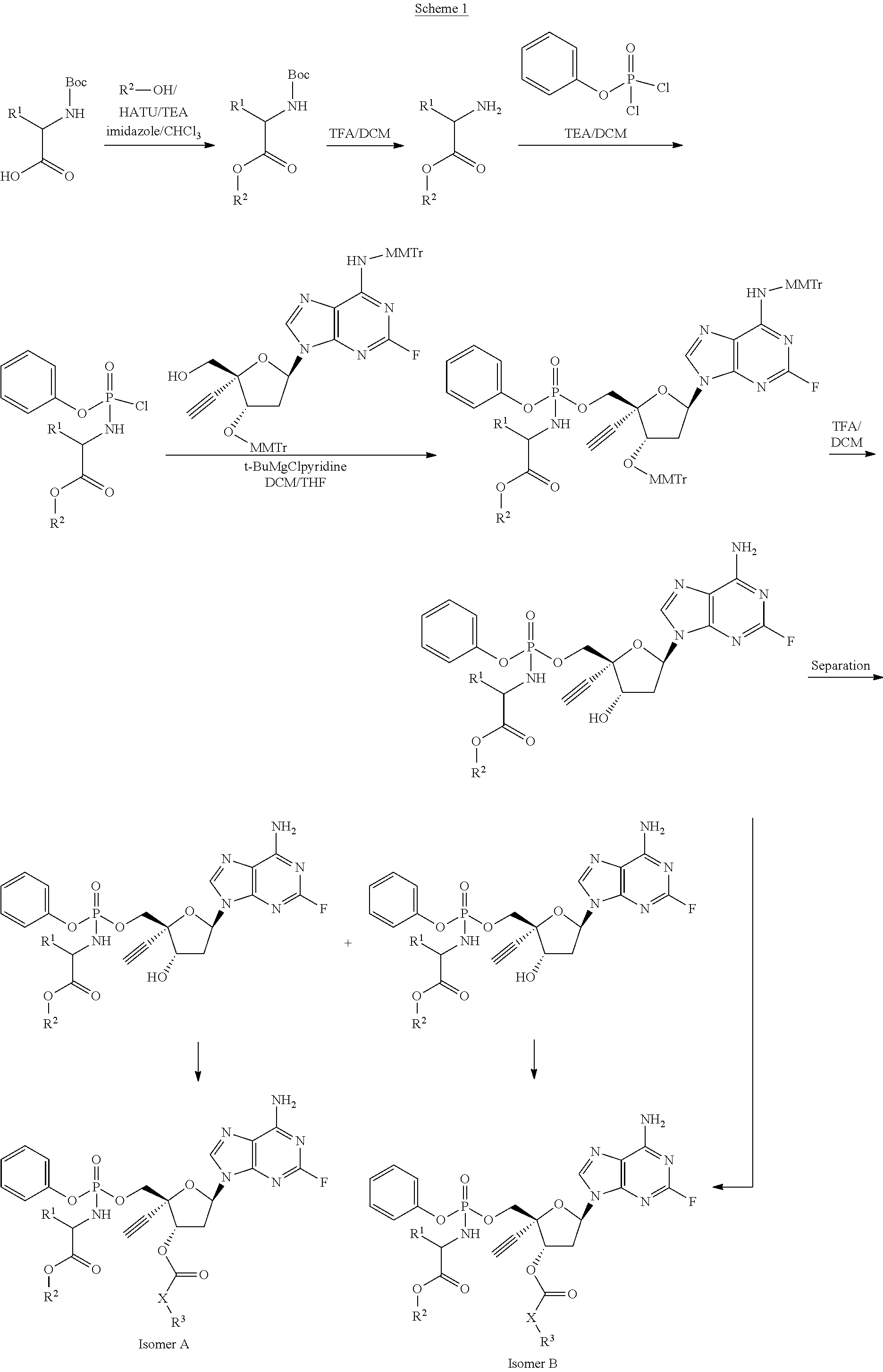

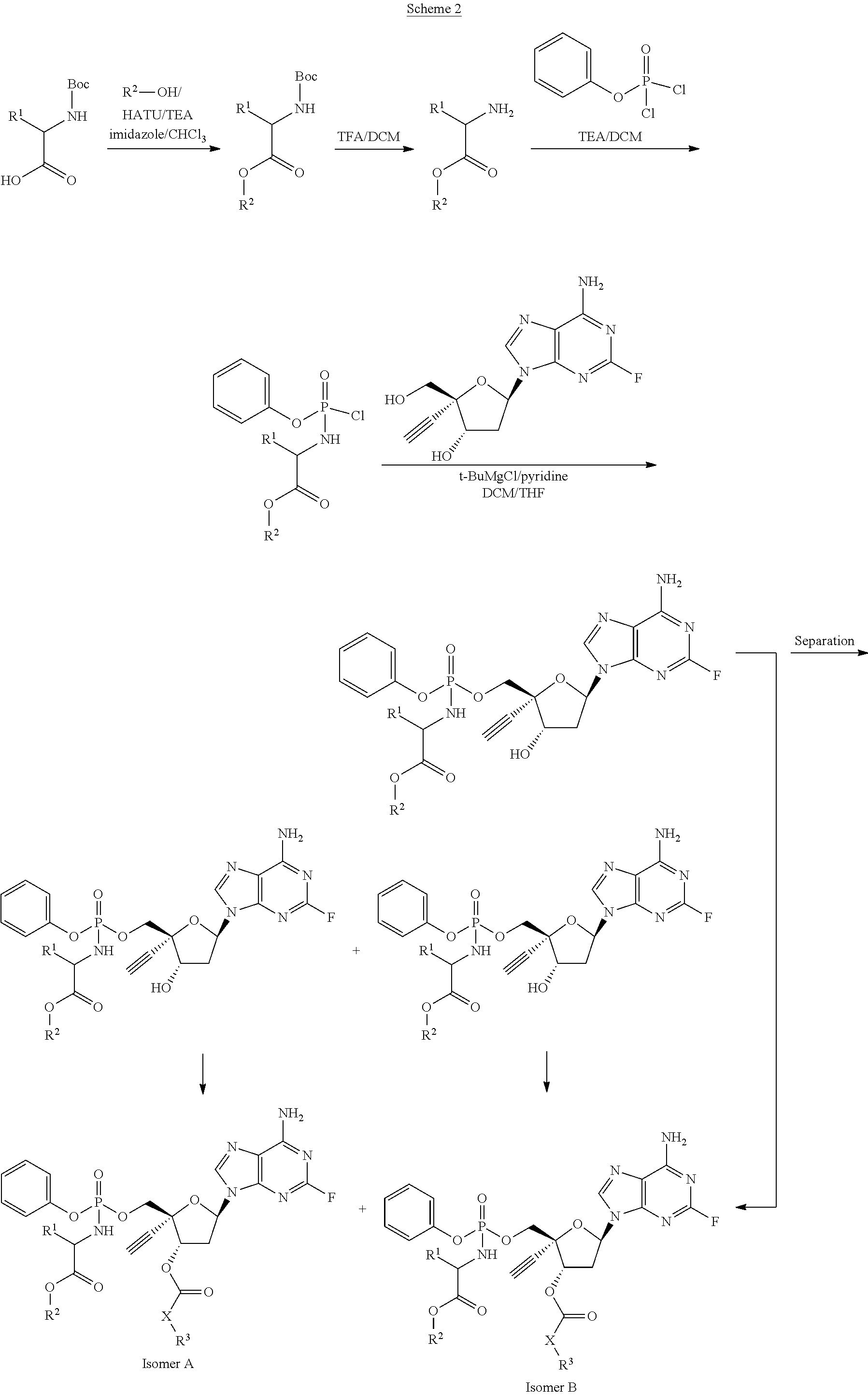

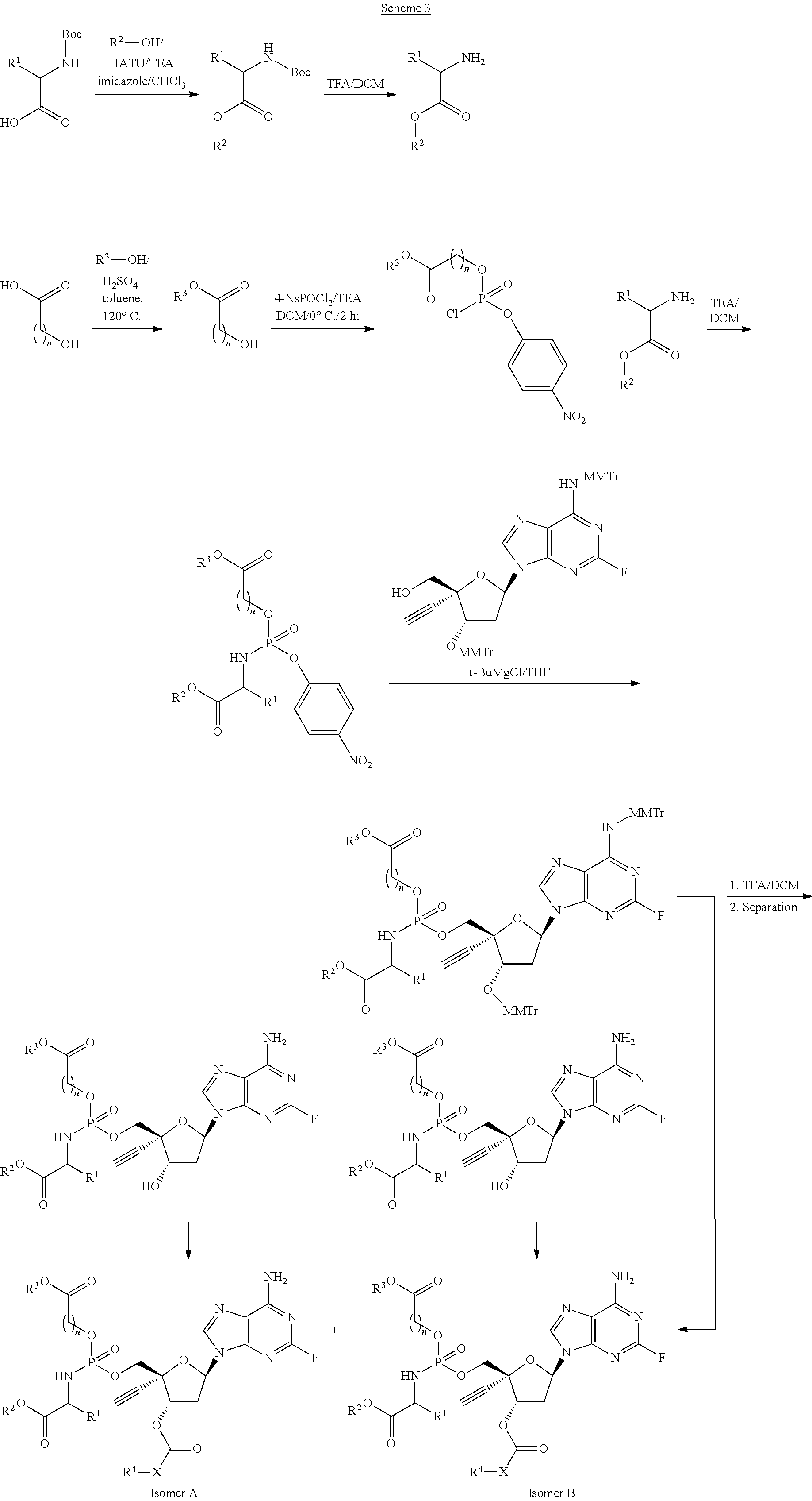

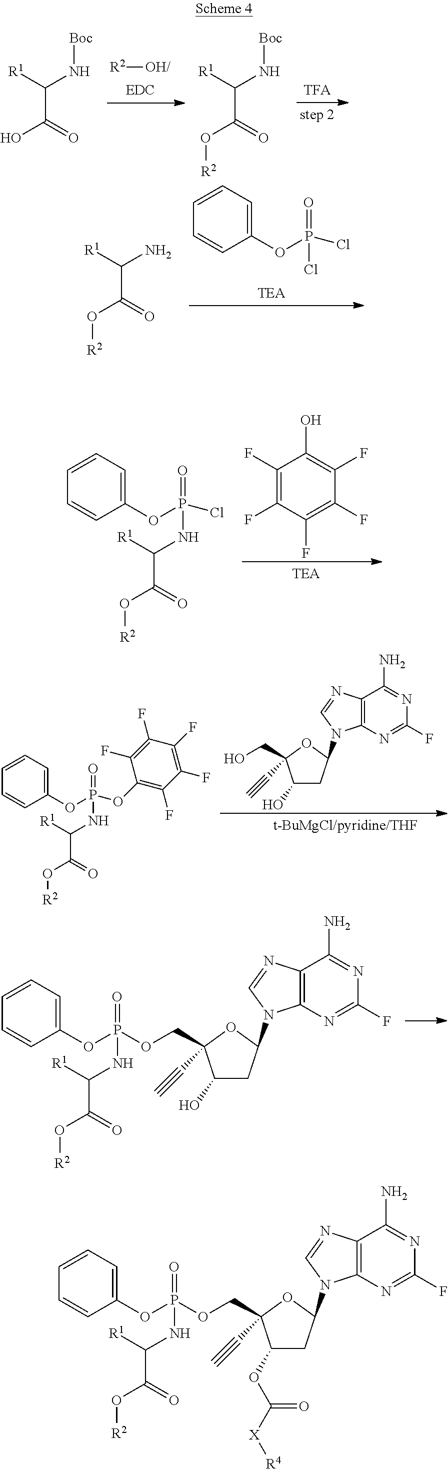

Absolute stereochemistry (R or S) of the phosphorus chiral center in each of the “A and “B” stereoisomers in Schemes 1, 2, 3 and 4 was not determined.

In Schemes 1, 2, 3 and 4 n is from 1 to 10, and each of, R¹, R², R³ and R⁴ are alkyl, alkylenearyl, or aryl, and wherein each of R¹, R², R³, and R⁴ may be independently and optionally substituted by one or more (C₁-C₁₄) alkyl, Cl, F, oxo, or (C₁-C₆) alkoxy.

### Example 1

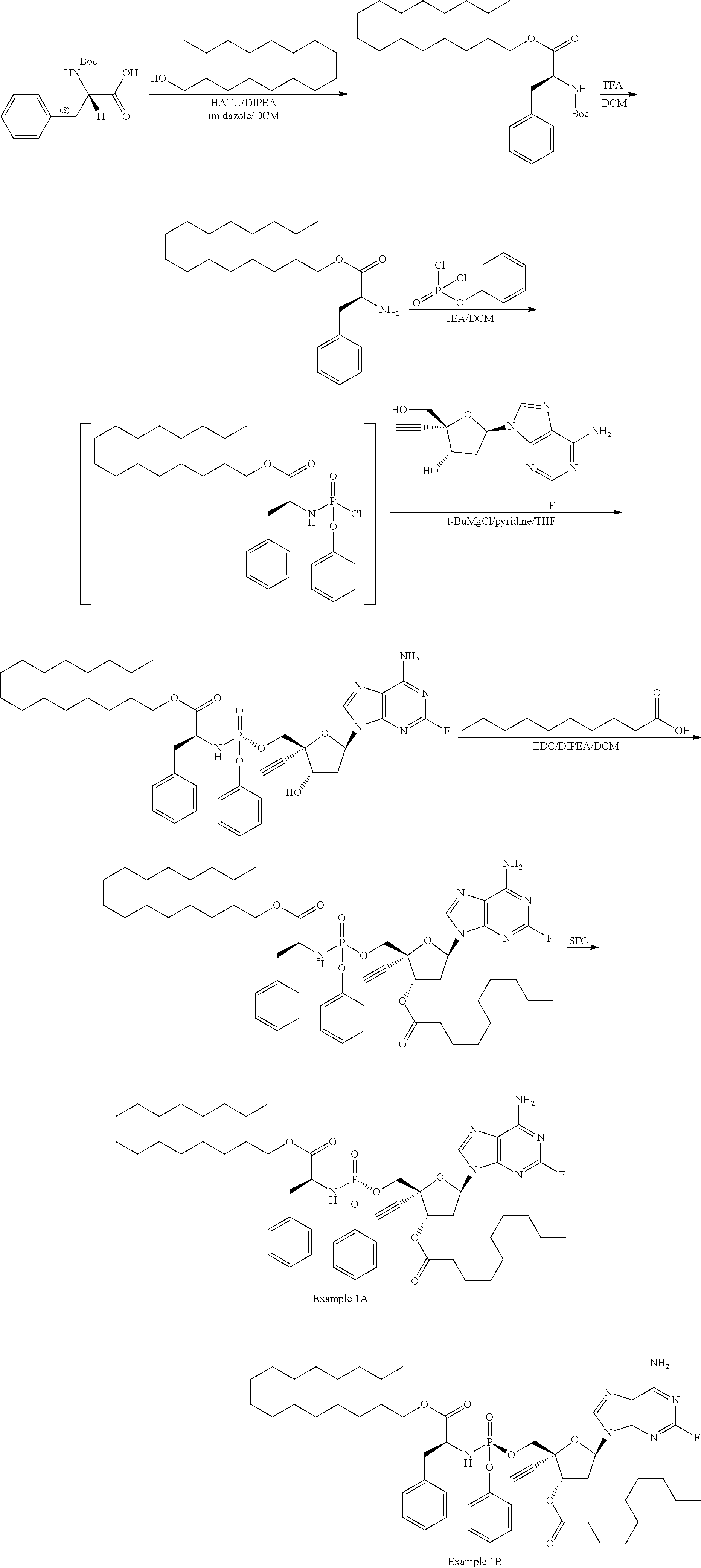

### Step 1: Hexadecyl (tert-butoxycarbonyl)-L-phenylalaninate

To a mixture of hexadecan-1-ol (15 g, 61.9 mmol), (tert-butoxycarbonyl)-L-phenylalanine (16.41 g, 61.9 mmol), 1H-imidazole (12.64 g, 186 mmol) and HATU (35.3 g, 93 mmol) in DCM (200 mL) was added DIPEA (32.4 mL, 186 mmol). The mixture was stirred at room temperature for 16 h. TLC (pet. ether:EtOAc=20:1, RF=0.5) showed reaction was completed. The mixture was washed with water, dried over Na₂SO₄, and concentrated to give crude product. The crude product was purified by flash silica gel chromatography (pet. ether:EtOAc=20:1) to give hexadecyl (tert-butoxycarbonyl)-L-phenylalaninate (23 g, 47.0 mmol, 76% yield) as a white solid.

### Step 2: Hexadecyl L-phenylalaninate

To a solution of hexadecyl (tert-butoxycarbonyl)-L-phenylalaninate (23 g, 47.0 mmol) in DCM (500 mL) was added trifluoroacetic acid (100 mL). The resulting mixture was stirred at room temperature for 12 h. TLC (pet. ether:EtOAc=10:1, RF=0.3) showed the reaction was completed. The reaction mixture was concentrated to remove DCM, and pH of the residue was adjusted to ˜8 with NaHCO₃(aq), extracted twice with EtOAc (100 ml). The organic phase was washed with water (100 ml), brine (100 mL), dried over Na₂SO₄ and concentrated to give crude product. The crude was purified by flash silica gel chromatography (pet. ether:EtOAc=5:1) to give hexadecyl L-phenylalaninate (18 g, 46.2 mmol, 98% yield) as a white solid. ¹H NMR (400 MHz, DMSO) δ 7.26 (t, J=7.4 Hz, 2H), 7.18 (t, J=8.4 Hz, 3H), 4.09-3.85 (m, 2H), 3.66 (t, J=6.8 Hz, 1H), 2.89-2.80 (m, 2H), 1.45 (dd, J=13.2, 6.5 Hz, 2H), 1.22 (s, 26H), 0.84 (t, J=6.7 Hz, 3H).

### Step 3: Hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold (ice-water bath) solution of hexadecyl L-phenylalaninate (2.66 g, 6.82 mmol), triethylamine (1.188 mL, 8.52 mmol) in DCM (6 mL) was added phenyl phosphorodichloridate (1.799 g, 8.52 mmol) in DCM (1 mL) dropwise under an atmosphere of nitrogen. Then, the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1 g, 3.41 mmol) in THF (20 mL)/pyridine (10 mL) was added tert-butylmagnesium chloride (8.52 mL, 8.52 mmol) dropwise under an atmosphere of nitrogen, then the mixture was stirred at room temperature for 30 min. The first prepared mixture was added dropwise to the second prepared mixture at room temperature and stirred for 2 h. LCMS indicated the presence of product. The mixture was filtered and concentrated to afford yellow solid. Then, the solid was dissolved in EtOAc (40 mL) and partitioned with HCl (0.05 N, 20 mL). The layers were separated and the aqueous layer was extracted with EtOAc (40 mL). The combined organic phases were washed with brine, dried over Na₂SO₄, filtered and concentrated to afford the crude product. The residue was purified by reverse phase chromatography (SepaFlash@ C18 column, BOSTON, 0-100% CH₃CN/10 mM NH₄HCO₃H2O) to obtain hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (1.3 g, 0.091 mmol, 2.66% yield) as a yellow solid. LCMS (M+H)=821.0; Retention time (0.1% TFA)=3.147 min.

### Step 4: (2R,3S,5R)-5-(6-Amino-2-fluoro-9H-purin-9-yl)-2-ethynyl-2-((((((S)-1-(hexadecyloxy)-1-oxo-3-phenylpropan-2-yl)amino)(phenoxy)phosphoryl)oxy)methyl)tetrahydrofuran-3-yl decanoate

To a solution of decanoic acid (185 mg, 1.072 mmol) in DCM (60 mL) was added DMAP (595 mg, 4.87 mmol) and EDC (934 mg, 4.87 mmol), and the resulting mixture was stirred for 0.5 h at room temperature. Then, hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (800 mg, 0.974 mmol) was added, and the resulting mixture was stirred overnight at room temperature. The LCMS showed the reaction was completed. The reaction mixture was quenched with water (40 mL) and extracted with DCM (30 mL×3). The combined organic phases were washed with brine (30 mL), dried over Na₂SO₄ and concentrated under vacuum. The residue was purified by reverse phase column (Boston, 120 g, 0-100% CH₃CN/10 mM NH₄HCO₃H2O) to afford (2R,3S,5R)-5-(6-amino-2-fluoro-9H-purin-9-yl)-2-ethynyl-2-((((((S)-1-(hexadecyloxy)-1-oxo-3-phenylpropan-2-yl)amino)(phenoxy)phosphoryl)oxy)methyl)tetrahydrofuran-3-yl decanoate (400 mg, 0.407 mmol, 41.7% yield). LCMS: Retention time (0.1% TFA)=4.626 min. The diastereomers were separated with Prep-SFC (Instrument: SFC-80 (Thar, Waters); Column: OZ 20×250 mm, 10 μm (Daicel); Column temperature: 40° C.; Mobile phase: CO₂/MEOH (0.5% ammonia/methanol)=45/55; Flow rate: 80 g/min; Back pressure: 100 bar; Detection wavelength: 260 nm; Cycle time: 10 min; Sample solution: 430 mg dissolved in 30 ml methanol; Injection volume: 2.2 ml) to afford first eluting isomer (Example 1A, RT: 1.62 min, 130 mg, 0.126 mmol, 30.7% yield) as white solid; LCMS: Retention time (0.1% TFA)=4.714 min; HPLC: Retention time (0.1% NH₄HCO₃)=15.833 min; ¹H NMR (400 MHz, CDCl₃) δ 8.04 (s, 1H), 7.28 (d, J=7.7 Hz, 1H), 7.25-7.19 (m, 4H), 7.16 (d, J=8.6 Hz, 2H), 7.12 (t, J=7.6 Hz, 1H), 7.03 (dd, J=7.5, 1.8 Hz, 2H), 6.43 (t, J=6.9 Hz, 1H), 6.09 (brs, 2H), 5.58 (dd, J=6.7, 3.8 Hz, 1H), 4.29 (dd, J=11.1, 6.7 Hz, 1H), 4.23 (ddd, J=15.1, 9.8, 5.3 Hz, 1H), 4.11 (dd, J=11.1, 5.9 Hz, 1H), 4.01 (td, J=6.8, 2.0 Hz, 2H), 3.71-3.60 (m, 1H), 2.97 (d, J=6.3 Hz, 2H), 2.64-2.48 (m, 3H), 2.38 (t, J=7.6 Hz, 2H), 1.82 (s, 2H), 1.70-1.60 (m, 2H), 1.25 (t, J=7.7 Hz, 38H), 0.87 (dt, J=4.1, 3.3 Hz, 6H) and second eluting isomer (Example 1B, RT: 2.68 min, 135 mg, 0.136 mmol, 33.2% yield) as white solid; LCMS: Retention time (0.1% TFA)=4.774 min; HPLC: Retention time (0.1% NH₄HCO₃)=14.483 min; ¹H NMR (400 MHz, CDCl₃) δ 8.00 (s, 1H), 7.21 (ddd, J=10.7, 6.9, 5.3 Hz, 5H), 7.15 (d, J=8.6 Hz, 2H), 7.13-7.08 (m, 3H), 6.39 (t, J=6.7 Hz, 1H), 6.12 (brs, 2H), 5.56 (dd, J=6.5, 4.4 Hz, 1H), 4.36-4.28 (m, 1H), 4.25 (dd, J=11.1, 6.3 Hz, 1H), 4.13 (dd, J=11.1, 5.7 Hz, 1H), 4.02 (t, J=6.8 Hz, 2H), 3.69 (t, J=10.7 Hz, 1H), 3.00 (d, J=6.5 Hz, 2H), 2.68-2.53 (m, 3H), 2.40 (t, J=7.6 Hz, 2H), 1.82 (s, 4H), 1.71-1.62 (m, 2H), 1.26 (t, J=6.8 Hz, 36H), 0.87 (dt, J=7.0, 3.3 Hz, 6H).

### Example 2

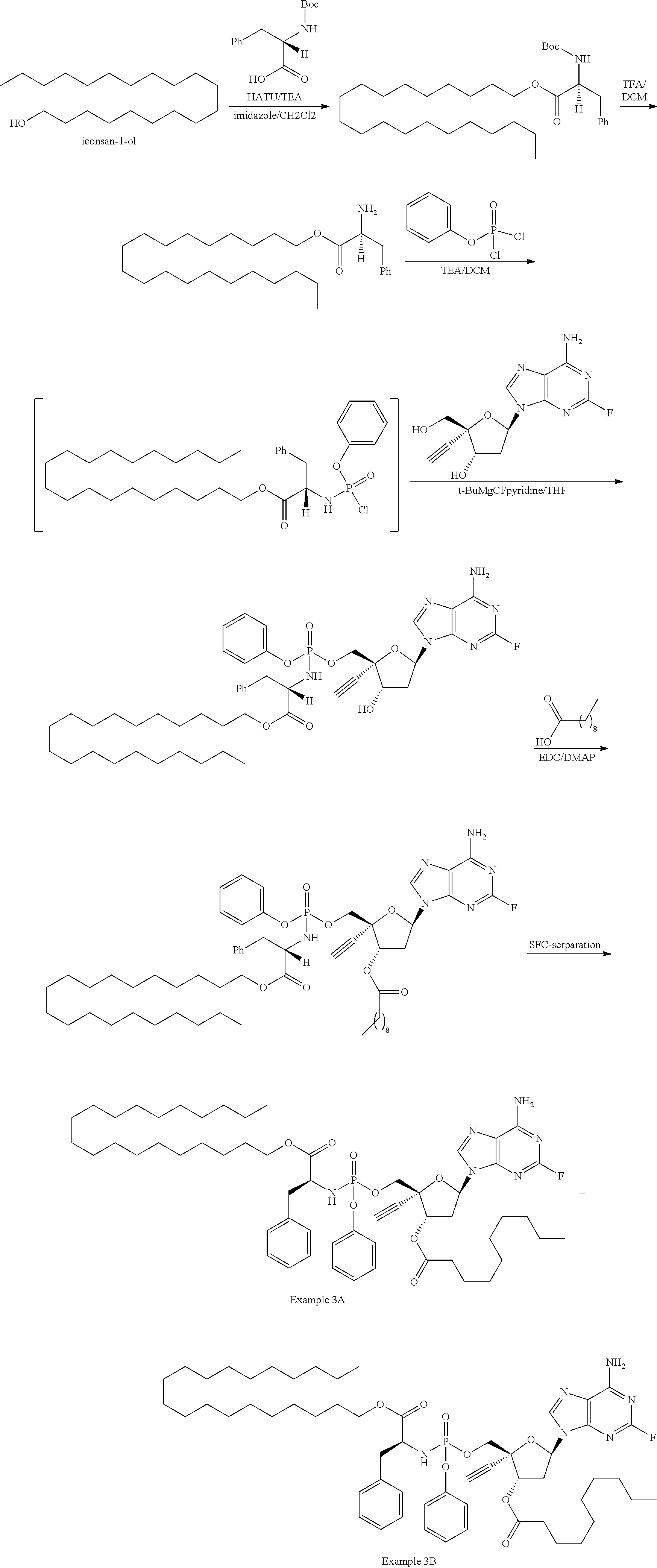

### Step 1: Octadecyl (tert-butoxycarbonyl)phenylalaninate

To a solution of octadecan-1-ol (32.5 g, 120 mmol), (tert-butoxycarbonyl)-L-phenylalanine (29 g, 109 mmol), and HATU (62.3 g, 164 mmol) in DCM (100 mL) was added 1H-imidazole (22.32 g, 328 mmol) and DIPEA (57.3 mL, 328 mmol). The resulting mixture was stirred at 25° C. overnight. TLC (Pet. ether:EtOAc=10:1, Rf=0.5) showed completion of reaction. The reaction was quenched with water (200 mL) and DCM (100 mL). Then, the organic layer was separated and the aqueous layer was extracted with DCM (2×50 mL). The combined organic layers were washed with brine (200 mL), dried over Na₂SO₄, filtered and concentrated to give crude product. The residue was purified by combiflash (silica gel column 330 g, hexane:ethyl acetate=20:1). The appropriate fractions containing desired product were combined and concentrated in vacuum to give octadecyl (tert-butoxycarbonyl)phenylalaninate (32 g, 61.8 mmol, 56.5% yield) as yellow solid.

### Step 2: Octadecyl phenylalaninate

To a solution of octadecyl (tert-butoxycarbonyl)phenylalaninate (32 g, 61.8 mmol) in DCM (400 mL) was added TFA (28.6 mL, 371 mmol). The resulting mixture was stirred at 25° C. for 2 h. TLC (pet. ether:EtOAc=10:1, Rf=0.1) showed the reaction was completed. The reaction was quenched with 1N NaHCO₃ (200 mL) and DCM (100 mL), then the organic layer was separated. The aqueous layer was extracted with DCM (2×100 mL). The combined organic layers were washed with brine (200 mL), dried over Na₂SO₄ and concentrated to give crude product octadecyl phenylalaninate (19 g, 45.5 mmol, 73.6% yield) which was used in the next step without purification.

### Step 3: Octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)phenylalaninate

To a solution of octadecyl L-phenylalaninate (5.70 g, 13.64 mmol) and triethylamine (1.901 mL, 13.64 mmol) in DCM (80 mL) was added phenyl phosphorodichloridate (2.038 mL, 13.64 mmol) in DCM (80 mL) dropwise under an atmosphere of nitrogen at about 5° C. Then, the reaction mixture was stirred at room temperature for 1 h. To a solution of (2R,3S,5R)-5-(6-amino-2-fluoro-9H-purin-9-yl)-2-ethynyl-2-(hydroxymethyl)tetrahydrofuran-3-ol (2 g, 6.82 mmol) in THF (40 mL)/pyridine (20 mL) was added tert-butylmagnesium chloride (17.05 mL, 17.05 mmol) dropwise under an atmosphere of nitrogen at 0° C. Then, the mixture was stirred at room temperature for 30 min. The first prepared mixture was added dropwise to the second prepared mixture at room temperature and stirred for 2 h. LCMS indicated completion of reaction. The mixture was filtered and concentrated to afford yellow solid. Then, the solid was dissolved in EtOAc (40 mL) and partitioned with HCl (0.05 N, 20 mL). The layers were separated, and the aqueous layer was extracted with EtOAc (40 mL). The combined organic phases were washed with brine, dried over Na₂SO₄, filtered and concentrated to afford the crude product. The residue was purified by reverse phase chromatography (SepaFlash@ C18, BOSTON, 0-100% CH₃CN/10 mM NH₄HCO₃H2O) to give octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)phenylalaninate (1.2 g, 1.354 mmol, 19.85% yield) as yellow solid. LCMS (M+H)=849.4; Retention time (0.1% TFA)=3.568 min. The diastereomers were separated by Prep-SFC (Instrument: SFC-150 (Waters), Column: IH 20×250 mm, 10 μm (Daicel), Column temperature: 35° C., Mobile phase: CO₂/MeOH (0.2% ammonia/methanol)=60/40, Flow rate: 100 g/min, Back pressure: 100 bar, Detection wavelength: 214 nm, Cycle time: 4 min, Sample solution: 1,900 mg dissolved in 100 mL methanol, Injection volume: 3 mL) to give first eluting isomer (Intermediate 2A, RT: 1.76 min, 800 mg, 0.908 mmol, 38.5% yield); LCMS (M+H)=849.4; Retention time (0.1% TFA)=2.874 min. HPLC: Retention time (0.1% NH₄HCO₃): 11.004 min; ¹H NMR (400 MHz, CDCl₃) δ 7.92 (s, 1H), 7.25-7.17 (m, 5H), 7.14 (d, J=7.3 Hz, 1H), 7.12-7.08 (m, 4H), 6.31 (dd, J=7.3, 4.3 Hz, 1H), 6.10 (s, 2H), 4.71 (t, J=7.6 Hz, 1H), 4.26 (dd, J=11.2, 7.7 Hz, 2H), 4.20 (dd, J=11.3, 8.8 Hz, 1H), 4.03 (t, J=6.7 Hz, 2H), 3.88 (s, 1H), 2.99 (d, J=6.2 Hz, 2H), 2.76-2.63 (m, 3H), 1.86 (s, 2H), 1.24 (d, J=9.0 Hz, 30H), 0.88 (t, J=6.8 Hz, 3H) and second eluting isomer (Intermediate 2B, RT: 3.21 min, 650 mg, 0.766 mmol, 32.5% yield) as white solid; LCMS (M+H)=849.5; Retention time (0.1% TFA)=2.839 min. HPLC: Retention time (0.1% NH₄HCO₃): 10.931 min; ¹H NMR (400 MHz, CDCl₃) δ 7.78 (s, 1H), 7.30-7.26 (m, 2H), 7.23 (dd, J=8.4, 4.6 Hz, 3H), 7.15 (d, J=7.8 Hz, 3H), 7.06 (dd, J=7.5, 1.7 Hz, 2H), 6.32 (dd, J=7.4, 4.2 Hz, 1H), 6.24 (s, 2H), 4.64 (t, J=7.6 Hz, 1H), 4.23-4.15 (m, 2H), 4.02 (t, J=6.7 Hz, 2H), 3.97 (dd, J=11.5, 8.4 Hz, 1H), 3.73 (t, J=10.8 Hz, 1H), 3.04-2.92 (m, 2H), 2.70 (s, 1H), 2.66 (dt, J=13.7, 7.8 Hz, 1H), 2.57 (ddd, J=13.6, 7.1, 4.2 Hz, 1H), 1.89 (s, 2H), 1.24 (d, J=4.9 Hz, 30H), 0.87 (t, J=6.8 Hz, 3H).

### Step 4: (2R,3S,5R)-5-(6-Amino-2-fluoro-9H-purin-9-yl)-2-ethynyl-2-((((((S)-1-(octadecyloxy)-1-oxo-3-phenylpropan-2-yl)amino)(phenoxy)phosphoryl)oxy)methyl)tetrahydrofuran-3-yl decanoate

To a solution of decanoic acid (67.0 mg, 0.389 mmol) in DCM (10 mL) were added DMAP (216 mg, 1.767 mmol) and EDC (339 mg, 1.767 mmol) and the resulting mixture was stirred for 0.5 h at room temperature. Then, octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 2A, 300 mg, 0.353 mmol) was added, the resulting mixture was stirred overnight at room temperature. The TLC showed the reaction was completed. The reaction mixture was diluted with DCM (60 mL), washed with water, brine, dried over Na₂SO₄, filtered and concentrated. The residue was purified by preparative thin layer chromatography (DCM/MeOH=10/1) to give (2R,3S,5R)-5-(6-amino-2-fluoro-9H-purin-9-yl)-2-ethynyl-2-((((((S)-1-(octadecyloxy)-1-oxo-3-phenylpropan-2-yl)amino)(phenoxy)phosphoryl)oxy)methyl)tetrahydrofuran-3-yl decanoate (Example 2A, 109 mg, 0.104 mmol, 29.5% yield) as colorless oil. HPLC Retention time (10 mM NH₄HCO₃)=15.68 min. ¹H NMR (400 MHz, CDCl₃) δ 8.01 (s, 1H), 7.25-7.19 (m, 4H), 7.17-7.14 (m, 3H), 7.13-7.08 (m, 3H), 6.40 (t, J=6.4 Hz, 1H), 6.08 (brs, 2H), 5.56 (dd, J=6.8, 4.8 Hz, 1H), 4.37-4.28 (m, 1H), 4.25 (dd, J=11.2, 6.4 Hz, 1H), 4.13 (dd, J=11.2, 6.0 Hz, 1H), 4.02 (t, J=6.8 Hz, 2H), 3.68 (t, J=10.4 Hz, 1H), 3.00 (d, J=6.4 Hz, 2H), 2.63-2.52 (m, 3H), 2.40 (t, J=7.2 Hz, 2H), 1.67 (quint, J=7.2 Hz, 2H), 1.52 (quint, J=6.4 Hz, 2H), 1.32-1.20 (m, 42H), 0.91-0.84 (m, 6H).

To a solution of decanoic acid (55.8 mg, 0.324 mmol) in DCM (15 mL) was added DMAP (180 mg, 1.472 mmol) and EDC (282 mg, 1.472 mmol). The resulting mixture was stirred for 0.5 h at room temperature. Then, octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 2B, 250 mg, 0.294 mmol) was added, and the resulting mixture was stirred overnight at room temperature. The TLC (DCM:MeOH=10:1, Rf=0.8) showed the reaction was completed. The reaction mixture was quenched with water (10 mL) and extracted with DCM (20 mL×3). The combined organic phases were washed with brine (30 mL), dried over Na₂SO₄ and concentrated under vacuum. The mixture was purified by preparative thin layer chromatography (DCM:CH₃OH=10:1, Rf=0.7) to give (2R,3S,5R)-5-(6-amino-2-fluoro-9H-purin-9-yl)-2-ethynyl-2-((((((S)-1-(octadecyloxy)-1-oxo-3-phenylpropan-2-yl)amino)(phenoxy)phosphoryl)oxy)methyl)tetrahydrofuran-3-yl decanoate (Example 2B, 140 mg, 0.140 mmol, 47.4% yield). HPLC: Retention time (10 mM NH₄HCO₃)=16.183 min. ¹H NMR (400 MHz, CDCl₃) δ 8.06 (s, 1H), 7.27 (d, J=7.5 Hz, 1H), 7.25-7.20 (m, 2H), 7.17 (dd, J=12.9, 7.0 Hz, 4H), 7.11 (d, J=7.1 Hz, 1H), 7.03 (dd, J=7.4, 1.8 Hz, 2H), 6.43 (t, J=6.8 Hz, 1H), 6.14 (s, 2H), 5.58 (dd, J=6.7, 3.8 Hz, 1H), 4.29 (dd, J=11.0, 6.8 Hz, 1H), 4.26-4.18 (m, 1H), 4.11 (dd, J=11.1, 5.9 Hz, 1H), 4.01 (td, J=6.8, 1.8 Hz, 2H), 3.67 (t, J=10.8 Hz, 1H), 2.97 (d, J=6.4 Hz, 2H), 2.64-2.56 (m, 2H), 2.52 (ddd, J=13.9, 6.2, 3.8 Hz, 1H), 2.38 (t, J=7.6 Hz, 2H), 1.92 (s, 2H), 1.69-1.61 (m, 2H), 1.30-1.21 (m, 42H), 0.87 (dt, J=7.0, 3.3 Hz, 6H).

### Example 3

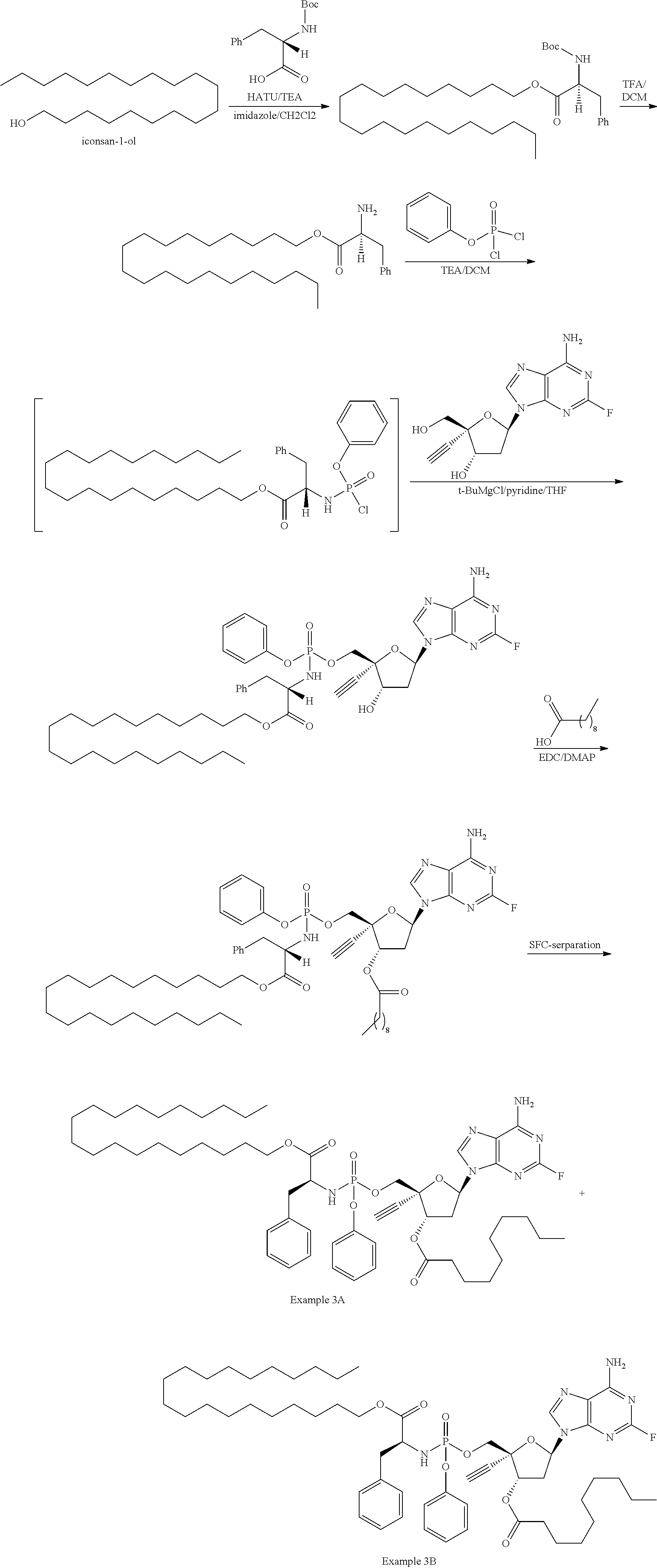

### Step 1: Icosyl (tert-butoxycarbonyl)-L-phenylalaninate

A mixture of icosan-1-ol (33.8 g, 113 mmol), (tert-butoxycarbonyl)-L-phenylalanine (25 g, 94 mmol), imidazole (19.25 g, 283 mmol), DIPEA (49.4 mL, 283 mmol) and HATU (53.7 g, 141 mmol) in DCM (200 mL) was stirred at 25° C. for 16 h. TLC showed the presence of new product. Water (200 mL) was added and the resulting mixture was extracted with DCM (150 mL×3). The combined organic phases were washed with brine, dried over Na₂SO₄, filtered and concentrated. The residue was purified by silica gel chromatography (pet. ether:EtOAc=30:1) to afford icosyl (tert-butoxycarbonyl)-L-phenylalaninate (20 g, 10.99 mmol, 11.67% yield) as white solid.

### Step 2: Icosyl L-phenylalaninate

To a solution of icosyl (tert-butoxycarbonyl)-L-phenylalaninate (30 g, 55.0 mmol) in DCM (200 mL) was added TFA (42.3 mL, 550 mmol) at 0° C. After 5 min, cold bath was removed and the mixture was stirred at room temperature for 2 h. TLC showed the presence of new compound. The reaction mixture was washed with water and sat. NaHCO₃. The combined aqueous layers were extracted with DCM (200 ml×3). The combined organic phases were washed with brine, dried over Na₂SO₄, filtered and concentrated to give the residue. The residue was purified by silica gel chromatography (DCM:MeOH=20:1) to give icosyl L-phenylalaninate (25 g, 50.5 mmol, 92% yield). ¹H NMR (400 MHz, DMSO) δ 7.40-7.10 (m, 5H), 4.0-3.85 (m, 2H), 3.55 (t, J=6.8 Hz, 1H), 2.92-2.71 (m, 2H), 1.91 (s, 1H), 1.54-1.38 (m, 2H), 1.23 (s, 33H), 0.85 (t, J=6.8 Hz, 3H).

### Step 3: Icosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold solution of icosyl L-phenylalaninate (7.60 g, 17.05 mmol), triethylamine (2.376 mL, 17.05 mmol) in DCM (48 mL) was added phenyl phosphorodichloridate (3.60 g, 17.05 mmol) in DCM (1 mL) dropwise under an atmosphere of nitrogen at 0° C., then the reaction mixture was stirred at room temperature for 1 h. To a cold solution of (2R,3S,5R)-5-(6-amino-2-fluoro-9H-purin-9-yl)-2-ethynyl-2-(hydroxymethyl)tetrahydrofuran-3-ol (2 g, 6.82 mmol) in THF (24 mL)/pyridine (12 mL) was added tert-butylmagnesium chloride (17.05 mL, 17.05 mmol) dropwise under an atmosphere of nitrogen at 0° C., then the reaction was stirred at room temperature for 30 min. The first prepared mixture was added dropwise to the second prepared mixture at room temperature and stirred for 2 h. LCMS indicated that starting material was consumed completely. The reaction mixture was filtered and the filtrate was concentrated. The residue was diluted with DCM and washed with 0.5 N HCl three times, brine, dried over Na₂SO₄, filtered and concentrated. The residue was purified by silica gel chromatography (DCM:MeOH=0-5%) to give icosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (800 mg, 0.867 mmol, 12.71% yield). LCMS (M+H)=876.0; Retention time (0.1% TFA)=4.30 min.

### Step 4: (2R,3S,5R)-5-(6-Amino-2-fluoro-9H-purin-9-yl)-2-ethynyl-2-((((((S)-1-(icosyloxy)-1-oxo-3-phenylpropan-2-yl)amino)(phenoxy)phosphoryl)oxy)methyl)tetrahydrofuran-3-yl decanoate

To a solution of icosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (1000 mg, 1.140 mmol) in DCM (10 mL) was added DMAP (139 mg, 1.140 mmol), DIEA (0.597 mL, 3.42 mmol) and EDC (1093 mg, 5.70 mmol). The resulting mixture was stirred for 0.5 h at room temperature. Then, decanoic acid (196 mg, 1.140 mmol) was added, and the reaction was stirred overnight at room temperature. TLC showed presence of new product. The reaction mixture was diluted with water and extracted with DCM (100 mL×3). The combined organic phases were washed with brine and dried over Na₂SO₄, filtered and concentrated to give the residue. The residue was purified by silica gel chromatography (DCM:MeOH=10:1) to (2R,3S,5R)-5-(6-amino-2-fluoro-9H-purin-9-yl)-2-ethynyl-2-((((((S)-1-(icosyloxy)-1-oxo-3-phenylpropan-2-yl)amino)(phenoxy)phosphoryl)oxy)methyl)tetrahydrofuran-3-yl decanoate (500 mg, 0.446 mmol, 39.1% yield). The diastereomers were separated with Prep-SFC (SFC-80 (Thar, Waters), Column: (R,R) Whelk-O1 20×250 mm, 10 μm (Daicel), Column temperature: 40° C., Mobile phase: CO₂/MEOH (0.2% ammonia/methanol)=45/55, Flow rate: 80 g/min, Back pressure: 100 bar, Detection wavelength: 260 nm, Cycle time:9 min) to give first eluting isomer (Example 3A, 120 mg, 0.114 mmol, 20.93% yield) as white solid; ¹H NMR (400 MHz, CDCl₃) δ 8.00 (s, 1H), 7.24-6.99 (m, 10H), 6.43 (t, J=6.9 Hz, 1H), 6.03 (s, 1H), 5.58 (dd, J=6.7, 3.7 Hz, 1H), 4.35-4.18 (m, 2H), 4.11 (dd, J=11.0, 5.8 Hz, 1H), 4.02 (dd, J=6.7, 5.1 Hz, 2H), 3.62 (t, J=10.8 Hz, 1H), 2.96 (d, J=6.2 Hz, 2H), 2.66-2.49 (m, 3H), 2.38 (t, J=7.6 Hz, 2H), 1.54 (d, J=6.0 Hz, 3H), 1.25 (t, J=7.1 Hz, 48H), 0.88 (dd, J=6.8, 5.4 Hz, 6H) and second eluting isomer (Example 3B, 110 mg, 0.107 mmol, 19.64% yield) as white solid; ¹H NMR (400 MHz, CDCl₃) δ 7.98 (s, 1H), 7.25-7.08 (m, 10H), 6.40 (t, J=6.7 Hz, 1H), 6.02 (s, 1H), 5.56 (dd, J=6.5, 4.5 Hz, 1H), 4.38-4.07 (m, 3H), 4.05-3.97 (m, 2H), 3.64 (t, J=10.7 Hz, 1H), 3.09-2.93 (m, 2H), 2.67-2.55 (m, 3H), 2.40 (t, J=7.6 Hz, 2H), 1.58-1.45 (m, 3H), 1.27 (d, J=9.3 Hz, 48H), 0.93-0.84 (m, 6H).

### Example 4

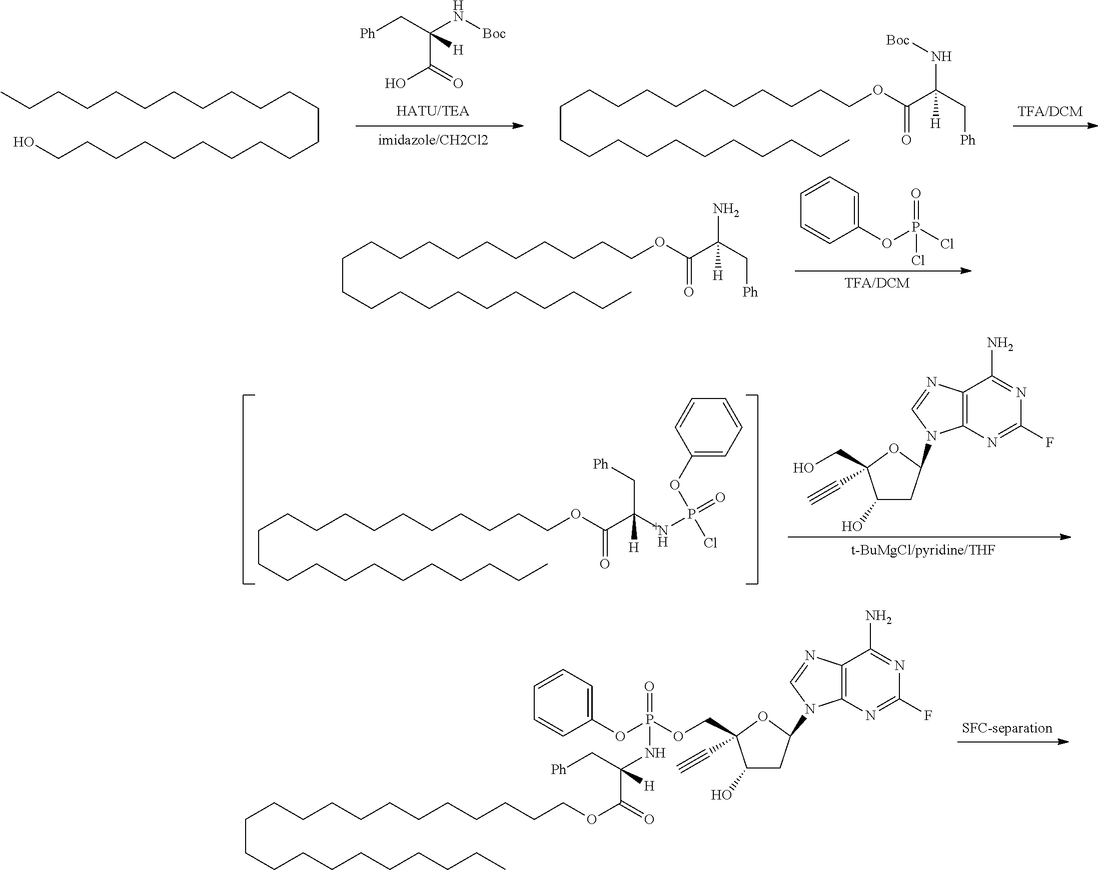

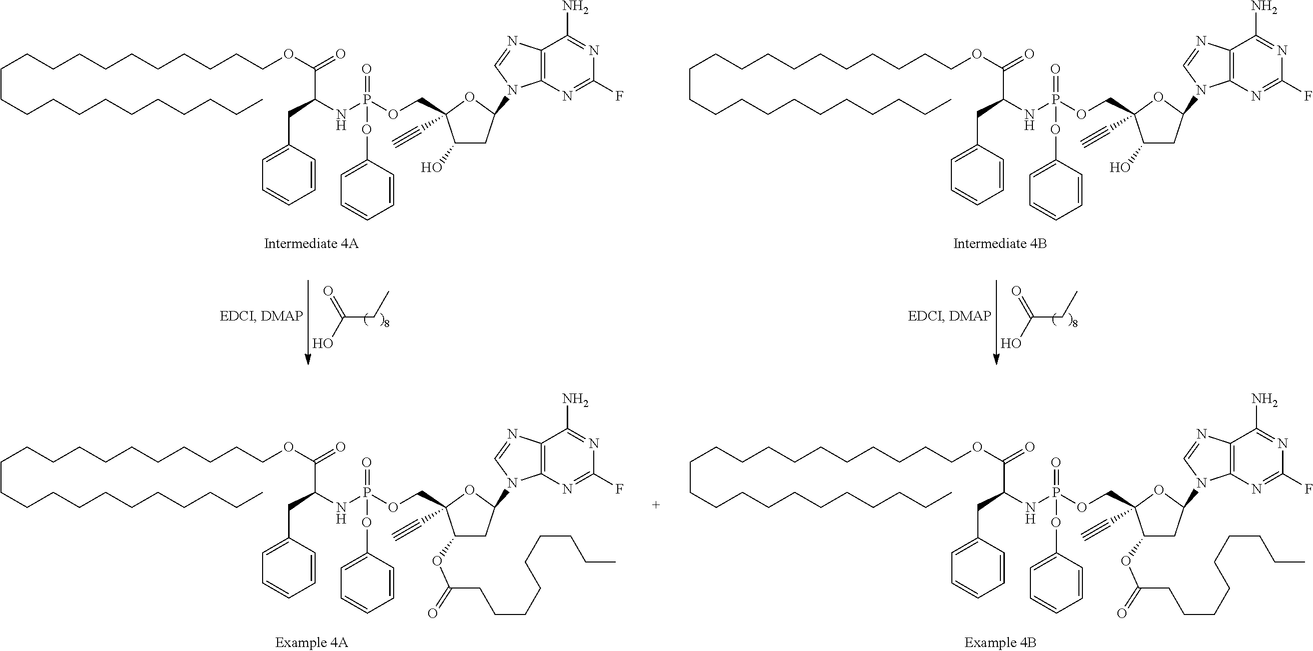

### Step 1: Docosyl (tert-butoxycarbonyl)-L-phenylalaninate

A mixture of docosan-1-ol (55.4 g, 170 mmol), (tert-butoxycarbonyl)-L-phenylalanine (50 g, 188 mmol), imidazole (38.5 g, 565 mmol), DIPEA (99 mL, 565 mmol) and HATU (107 g, 283 mmol) in DCM (500 mL) was stirred at 25° C. for 16 h. TLC showed the presence of new compound. Water (500 mL) was added and the mixture was extracted with DCM (250 mL×3). The combined organic phases were washed with brine, dried over Na₂SO₄ and concentrated. The residue was purified by silica gel chromatography (pet. ether:EtOAc=20:1) to afford docosyl (tert-butoxycarbonyl)-L-phenylalaninate (48 g, 79 mmol, 42.2% yield) as white solid. ¹H NMR (400 MHz, CDCl₃) δ 7.33-7.23 (m, 3H), 7.13 (d, J=7.0 Hz, 2H), 4.98 (d, J=7.9 Hz, 1H), 4.57 (d, J=7.0 Hz, 1H), 4.18-4.01 (m, 2H), 3.20-2.96 (m, 2H), 1.68-1.52 (m, 2H), 1.42 (s, 9H), 1.26 (s, 38H), 0.88 (t, J=6.8 Hz, 3H).

### Step 2: Docosyl L-phenylalaninate

To a cold (ice-water bath) solution of docosyl (tert-butoxycarbonyl)-L-phenylalaninate (48 g, 84 mmol) in DCM (500 mL) was added TFA (64.4 mL, 836 mmol). After 5 min, the mixture was removed from cold bath and stirred at room temperature for 2 h. TLC showed completion of reaction. The reaction mixture was washed with water and sat. Na₂CO₃. The combined aqueous layers were extracted with DCM (400 ml×3), and combined organic layers were washed with brine, dried over Na₂SO₄, filtered and concentrated to give the residue. The residue was purified by silica gel chromatography (DCM:MeOH=20:1) to give docosyl L-phenylalaninate (36 g, 72.2 mmol, 86% yield). ¹H NMR (400 MHz, DMSO) δ 7.40-7.10 (m, 5H), 4.07-3.85 (m, 2H), 3.55 (t, J=6.8 Hz, 1H), 2.92-2.71 (m, 2H), 1.91 (s, 1H), 1.54-1.38 (m, 2H), 1.23 (s, 38H), 0.85 (t, J=6.8 Hz, 3H).

### Step 3: Docosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold (ice-water bath) solution of docosyl L-phenylalaninate (2019 mg, 4.26 mmol), triethylamine (0.238 mL, 1.705 mmol) in DCM (48 mL) was added phenyl phosphorodichloridate (899 mg, 4.26 mmol) in DCM (2 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (500 mg, 1.705 mmol) in THF (20 mL)/pyridine (10 mL) was added tert-butylmagnesium chloride (3.41 mL, 3.41 mmol) dropwise under an atmosphere of nitrogen, then the mixture was stirred at room temperature for 30 min. The first prepared mixture was added dropwise to the second prepared mixture at room temperature and stirred for 2 h. LCMS indicated completion of reaction. The reaction mixture was filtered and the filtrate was concentrated. The residue was diluted with DCM, and washed with 0.5 N HCl three times and brine, dried over Na₂SO₄, filtered and concentrated to give residue. The residue was purified by flash chromatography (0-5% MeOH/DCM) to give docosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (400 mg, 0.442 mmol, 25.9% yield. LCMS (M+H)=905.7; Retention time (0.1% TFA)=4.39 min. The diastereomers were separated with Prep-SFC (SFC-80 (Thar, Waters), Column: (R,R) Whelk-O1 20×250 mm, 10 μm (Daicel), Column temperature: 40° C., Mobile phase: CO₂/MEOH (0.2% ammonia/methanol)=45/55, Flow rate: 80 g/min, Back pressure: 100 bar, Detection wavelength: 260 nm, Cycle time:9 min) to give first eluting isomer docosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 4A, 1.2 g, 1.326 mmol, 40.0% yield) as white solid; LCMS (M+H)=906.7 and second eluting isomer docosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 4B, 1.5 g, 1.657 mmol, 50.0% yield); LCMS (M+H)=906.7

### Step 4: (2R,3S,5R)-5-(6-amino-2-fluoro-9H-purin-9-yl)-2-((((((S)-1-(docosyloxy)-1-oxo-3-phenylpropan-2-yl)amino)(phenoxy)phosphoryl)oxy)methyl)-2-ethynyltetrahydrofuran-3-yl decanoate

To a solution of docosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 4A, 400 mg, 0.442 mmol) in DCM (10 mL) was added DMAP (54.0 mg, 0.442 mmol), EDC (254 mg, 1.326 mmol) and DIEA (0.232 mL, 1.326 mmol). The resulting mixture was stirred for 0.5 h at room temperature. Then decanoic acid (76 mg, 0.442 mmol) was added and the resulting mixture was stirred overnight at room temperature. TLC showed the presence of new compound. The reaction mixture was diluted with water and extracted with DCM (20 mL×3). The combined organic phases were washed with brine and dried over Na₂SO₄, filtered and concentrated to give the residue. The residue was purified by flash chromatography (0-10% MeOH/DCM) to afford (2R,3S,5R)-5-(6-amino-2-fluoro-9H-purin-9-yl)-2-((((((S)-1-(docosyloxy)-1-oxo-3-phenylpropan-2-yl)amino)(phenoxy)phosphoryl)oxy)methyl)-2-ethynyltetrahydrofuran-3-yl decanoate (Example 4A, 260 mg, 0.239 mmol, 54.0% yield). ¹H NMR (400 MHz, CDCl₃) δ 7.98 (s, 1H), 7.26-7.05 (m, 10H), 6.40 (t, J=6.7 Hz, 1H), 6.09 (s, 1H), 5.56 (dd, J=6.5, 4.4 Hz, 1H), 4.39-4.19 (m, 2H), 4.13 (dd, J=11.1, 5.6 Hz, 1H), 4.02 (t, J=6.8 Hz, 2H), 3.76-3.60 (m, 1H), 3.11-2.94 (m, 2H), 2.67-2.57 (m, 3H), 2.39 (d, J=7.7 Hz, 2H), 1.66 (dd, J=14.7, 7.3 Hz, 2H), 1.50 (dd, J=20.8, 14.5 Hz, 2H), 1.36-1.20 (m, 51H), 0.93-0.78 (m, 6H).

To a solution docosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 4B, 400 mg, 0.442 mmol) in DCM (10 mL) was added DMAP (54.0 mg, 0.442 mmol), EDC (254 mg, 1.326 mmol) and DIEA (0.232 mL, 1.326 mmol). The resulting mixture was stirred for 0.5 h at room temperature. Then, decanoic acid (76 mg, 0.442 mmol) was added, and the resulting mixture was stirred overnight at room temperature. TLC showed the presence of new compound. The reaction mixture was diluted with water and extracted with DCM (20 mL×3). The combined organic phases were washed with brine and dried over Na₂SO₄, filtered and concentrated to give the residue. The residue was purified by flash chromatography (0-10% MeOH/DCM) to afford (2R,3S,5R)-5-(6-amino-2-fluoro-9H-purin-9-yl)-2-((((((S)-1-(docosyloxy)-1-oxo-3-phenylpropan-2-yl)amino)(phenoxy)phosphoryl)oxy)methyl)-2-ethynyltetrahydrofuran-3-yl decanoate (Example 4B, 200 mg, 0.188 mmol, 42.5% yield). ¹H NMR (400 MHz, CDCl₃) δ 8.01 (s, 1H), 7.25-6.96 (m, 10H), 6.43 (t, J=6.9 Hz, 1H), 6.08 (s, 1H), 5.58 (dd, J=6.7, 3.8 Hz, 1H), 4.32-4.18 (m, 2H), 4.09 (dt, J=14.2, 7.1 Hz, 1H), 4.06-3.94 (m, 2H), 3.67 (dt, J=21.6, 10.9 Hz, 1H), 2.95 (t, J=9.9 Hz, 2H), 2.66-2.49 (m, 3H), 2.38 (t, J=7.6 Hz, 2H), 1.64 (dd, J=14.6, 7.3 Hz, 2H), 1.54 (d, J=6.5 Hz, 2H), 1.38-1.17 (m, 51H), 0.97-0.81 (m, 6H).

### Example 5

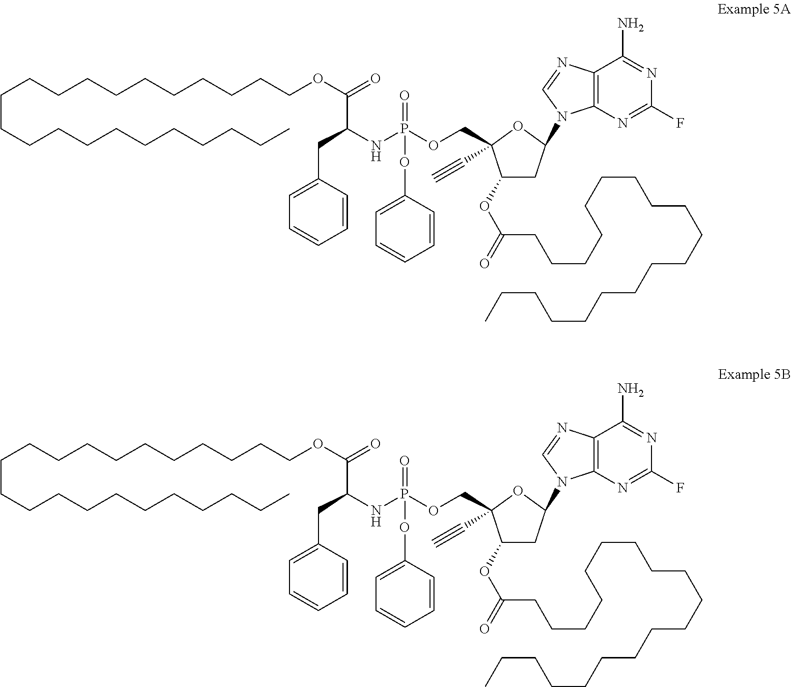 [SMILES: C#C[C@]1(COP(=O)(N[C@@H](Cc2ccccc2)C(=O)OCCCCCCCCCCCCCCCCCCCCCC)Oc2ccccc2)O[C@@H](n2cnc3c(N)nc(F)nc32)C[C@@H]1OC(=O)CCCCCCCCCCCCCCCCCCC ; C#C[C@]1(COP(=O)(N[C@@H](Cc2ccccc2)C(=O)OCCCCCCCCCCCCCCCCCCCCCC)Oc2ccccc2)O[C@@H](n2cnc3c(N)nc(F)nc32)C[C@@H]1OC(=O)CCCCCCCCCCCCCCCCCCC]

### (2R,3S,5R)-5-(6-amino-2-fluoro-9H-purin-9-yl)-2-((((((S)-1-(docosyloxy)-1-oxo-3-phenylpropan-2-yl)amino)(phenoxy)phosphoryl)oxy)methyl)-2-ethynyltetrahydrofuran-3-yl icosanoate

To a solution of docosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 4A, 400 mg, 0.442 mmol) in DCM (10 mL) was added DMAP (54.0 mg, 0.442 mmol), EDC (254 mg, 1.326 mmol), DIEA (0.232 mL, 1.326 mmol) and icosanoic acid (138 mg, 0.442 mmol). The resulting mixture was stirred overnight at room temperature. TLC showed the presence of new compound. The reaction mixture was diluted with water and extracted with DCM (20 mL×3). The combined organic phases were washed with brine, dried over Na₂SO₄, filtered and concentrated to give the residue. The residue was purified by flash chromatography (0-10% MeOH/DCM) to afford (2R,3S,5R)-5-(6-amino-2-fluoro-9H-purin-9-yl)-2-((((((S)-1-(docosyloxy)-1-oxo-3-phenylpropan-2-yl)amino)(phenoxy)phosphoryl)oxy)methyl)-2-ethynyltetrahydrofuran-3-yl icosanoate (Example 5A, 220 mg, 0.183 mmol, 41.5% yield). ¹H NMR (400 MHz, CDCl₃) δ 8.02 (s, 1H), 7.25-7.03 (m, 10H), 6.40 (t, J=6.7 Hz, 1H), 6.21 (s, 1H), 5.57 (dd, J=6.4, 4.4 Hz, 1H), 4.42-4.21 (m, 2H), 4.14 (dd, J=11.0, 5.7 Hz, 1H), 4.02 (t, J=6.8 Hz, 2H), 3.75 (dt, J=30.9, 10.5 Hz, 1H), 3.07-2.92 (m, 2H), 2.69-2.56 (m, 3H), 2.40 (t, J=7.6 Hz, 2H), 1.74-1.59 (m, 2H), 1.56-1.45 (m, 2H), 1.38-1.17 (m, 71H), 0.88 (t, J=6.8 Hz, 6H).

To a solution of docosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 4B, 400 mg, 0.442 mmol) in DCM (20 mL) was added DMAP (54.0 mg, 0.442 mmol), EDC (254 mg, 1.326 mmol), DIEA (0.232 mL, 1.326 mmol) and icosanoic acid (138 mg, 0.442 mmol). The resulting mixture was stirred overnight at room temperature. TLC showed the presence of new compound. The reaction mixture was diluted with water and extracted with DCM (20 mL×3). The combined organic phases were washed with brine, dried over Na₂SO₄, filtered and concentrated to give the residue. The residue was purified by flash chromatography (0-10% MeOH/DCM) to afford (2R,3S,5R)-5-(6-amino-2-fluoro-9H-purin-9-yl)-2-((((((S)-1-(docosyloxy)-1-oxo-3-phenylpropan-2-yl)amino)(phenoxy)phosphoryl)oxy)methyl)-2-ethynyltetrahydrofuran-3-yl icosanoate (Example 5B, 160 mg, 0.133 mmol, 30.2% yield). ¹H NMR (400 MHz, CDCl₃) δ 8.01 (s, 1H), 7.24-6.98 (m, 10H), 6.43 (t, J=6.9 Hz, 1H), 6.10 (s, 1H), 5.59 (dd, J=6.7, 3.7 Hz, 1H), 4.26 (ddd, J=25.7, 13.2, 6.4 Hz, 2H), 4.11 (dd, J=11.0, 5.8 Hz, 1H), 4.01 (dd, J=6.7, 5.1 Hz, 2H), 3.68 (t, J=10.8 Hz, 1H), 2.95 (t, J=10.4 Hz, 2H), 2.66-2.49 (m, 3H), 2.38 (t, J=7.5 Hz, 2H), 1.64 (dd, J=14.5, 7.2 Hz, 2H), 1.50 (d, J=17.8 Hz, 2H), 1.37-1.16 (m, 71H), 0.88 (t, J=6.8 Hz, 6H).

### Example 6

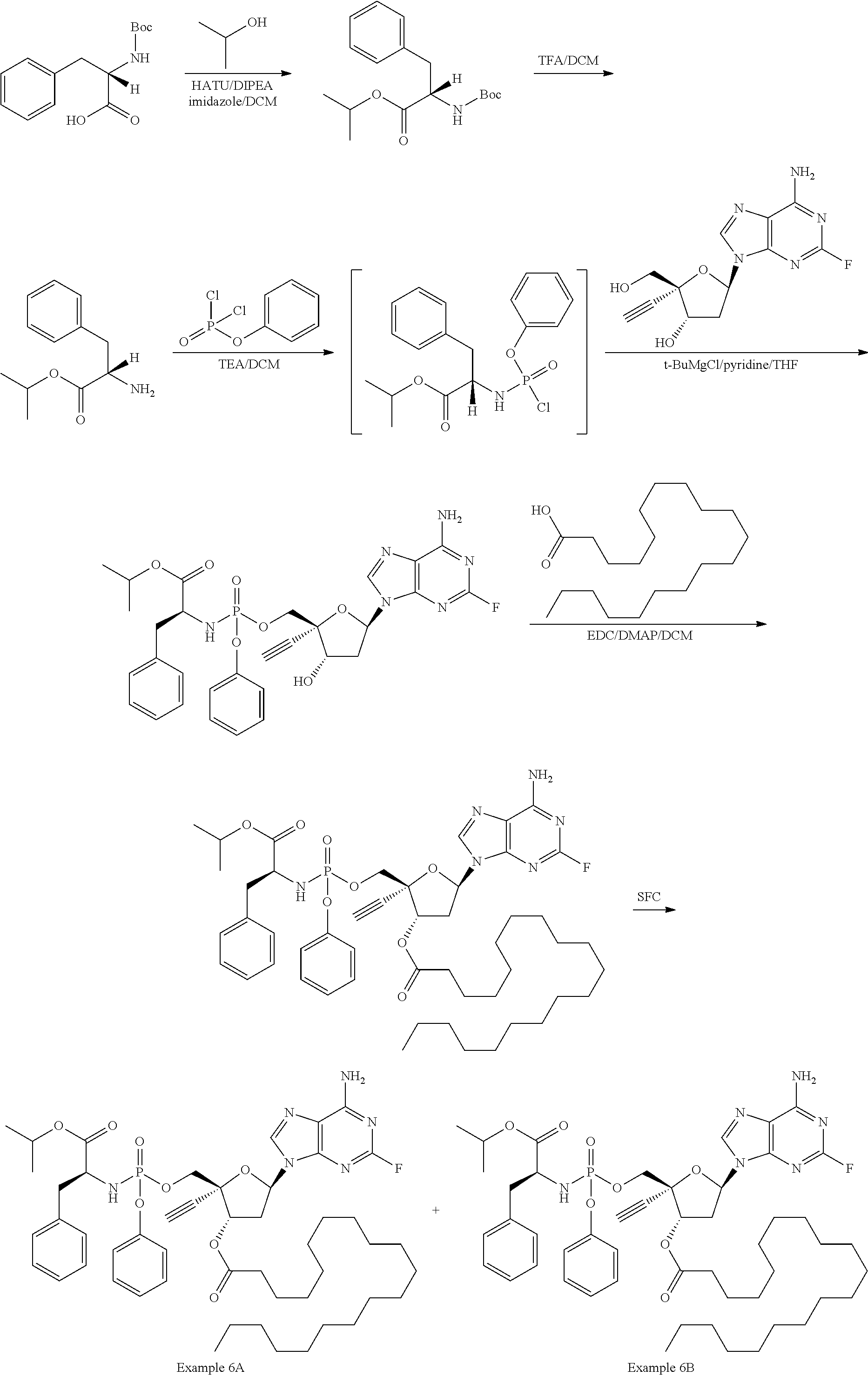

### Step 1: Isopropyl (tert-butoxycarbonyl)-L-phenylalaninate

To a mixture of propan-2-ol (4.53 g, 75 mmol), (tert-butoxycarbonyl)-L-phenylalanine (20 g, 75 mmol) and HATU (34.4 g, 90 mmol) in DCM (300 mL) was added 1H-imidazole (15.40 g, 226 mmol) and triethylamine (31.4 mL, 226 mmol). The resulting mixture was stirred at 25° C. overnight. TLC showed the reaction was completed. The reaction was quenched with water (100 mL) and diluted with DCM (100 mL) then the organic layer was separated. The aqueous layer was extracted with DCM (2×200 mL). The combined organic layers were washed with brine (100 mL), dried over Na₂SO₄, filtered and concentrated. The residue was purified by silica gel column chromatography (hexane/ethyl acetate=10/1) to give isopropyl (tert-butoxycarbonyl)-L-phenylalaninate (22.7 g, 73.8 mmol, 98% yield) as a white solid. LCMS (M+Na)=330.0; Retention time (0.1% TFA)=2.08 min.

### Step 2: Isopropyl L-phenylalaninate

To a solution of isopropyl (tert-butoxycarbonyl)-L-phenylalaninate (22.7 g, 73.8 mmol) in DCM (120 mL) was added TFA (40 mL, 519 mmol) at 0° C. The resulting mixture was stirred at 25° C. for 4 h. LCMS showed the reaction was completed. The reaction mixture was concentrated under reduced pressure. The residue was neutralized with NH₄HCO₃ (20 mL), diluted with water (200 mL) and extracted with DCM (3×200 mL). Then the combined organic layers were washed with brine (100 mL), dried over Na₂SO₄, filtered and concentrated. The residue was purified by silica gel chromatography (DCM/MeOH=10/1) to give isopropyl L-phenylalaninate (12.5 g, 60.3 mmol, 82% yield) as a yellow oil. LCMS (M+H)=208.0; Retention time (0.1% TFA)=1.13 min.

### Step 3: Isopropyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a mixture of isopropyl L-phenylalaninate (5.30 g, 25.6 mmol), triethylamine (3.56 mL, 25.6 mmol) in DCM (60 mL) was added phenyl phosphorodichloridate (3.82 mL, 25.6 mmol) in DCM (1 mL) dropwise under an atmosphere of nitrogen at 0° C., then the reaction mixture was stirred at the same temperature for 1 h. To a solution of (2R,3S,5R)-5-(6-amino-2-fluoro-9H-purin-9-yl)-2-ethynyl-2-(hydroxymethyl)tetrahydrofuran-3-ol (3 g, 10.23 mmol) in THF (20 mL) and pyridine (10 mL) was added tert-butylmagnesium chloride (25.6 mL, 25.6 mmol) dropwise under an atmosphere of nitrogen at 0° C. Then, the mixture was stirred at the same temperature for 30 min. The first prepared mixture was added dropwise to the second prepared mixture at 0° C. The mixture was stirred at 10° C. for 2 h. LCMS showed the reaction was completed. The reaction mixture was quenched with 2N NH₄Cl (10 mL) and diluted with EtOAc (200 mL). The organic layer was washed with water (50 mL), brine (50 mL), dried over Na₂SO₄, filtered and concentrated. The residue was purified by reverse phase chromatography (SepaFlash@ C18, BOSTON, 0-100% CH₃CN/10 mM NH₄HCO₃H2O) to give isopropyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (1 g, 1.368 mmol, 13.37% yield) as a white solid. LCMS (M+H)=639.0; Retention time (10 mM NH₄HCO₃)=1.67 min.

### Step 4: (2R,3S,5R)-5-(6-Amino-2-fluoro-9H-purin-9-yl)-2-ethynyl-2-((((((S)-1-isopropoxy-1-oxo-3-phenylpropan-2-yl)amino)(phenoxy)phosphoryl)oxy)methyl)tetrahydrofuran-3-yl icosanoate

To a solution of icosanoic acid (323 mg, 1.034 mmol) in DCM (40 mL) were added EDC (901 mg, 4.70 mmol) and DMAP (574 mg, 4.70 mmol), and the resulting mixture was stirred for 0.5 h at room temperature. Then isopropyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (600 mg, 0.940 mmol) was added and the resulting mixture was stirred overnight at room temperature. LCMS showed the reaction was completed. The reaction mixture was diluted with DCM (200 mL), organic layer separated, and washed with water (40 mL), brine (40 mL), dried over Na₂SO₄, filtered and concentrated. The residue was purified by reverse phase chromatography (SepaFlash@ C18, BOSTON, 0-100% CH₃CN/10 mM NH₄HCO₃H2O and 100% THF) to give (2R,3S,5R)-5-(6-amino-2-fluoro-9H-purin-9-yl)-2-ethynyl-2-((((((S)-1-isopropoxy-1-oxo-3-phenylpropan-2-yl)amino)(phenoxy)phosphoryl)oxy)methyl)tetrahydrofuran-3-yl icosanoate (600 mg, 0.643 mmol, 68.41% yield) as a colorless oil. The diastereomers were separated with Prep-SFC (Instrument: SFC-80 (Thar, Waters), Column: IG 20×250 mm, 10 μm (Daicel), Column temperature: 40° C., Mobile phase: CO₂/EtOH (1.0% ammonia/methanol)=35/65, Flow rate: 80 g/min, Back pressure: 100 bar, Detection wavelength: 260 nm, Cycle time: 15 min) to give first eluting isomer (Example 6A, RT1: 1.57 min, 135 mg, 0.145 mmol, yield: 15.40%) as light grey solid; LCMS (M+H)=933.5; Retention time (0.05% TFA)=4.05 min. HPLC Retention time (10 mM NH₄HCO₃)=14.89 min. ¹H NMR (400 MHz, CDCl₃) δ 8.06 (s, 1H), 7.22-7.10 (m, 7H), 7.08-7.04 (m, 2H), 6.43 (t, J=6.8 Hz, 1H), 6.04 (brs, 2H), 5.57 (dd, J=6.8, 4.0 Hz, 1H), 4.94 (quint, J=6.4 Hz, 1H), 4.29 (dd, J=10.8, 6.8 Hz, 1H), 4.22-4.15 (m, 1H), 4.09 (dd, J=10.8, 5.6 Hz, 1H), 3.60 (t, J=10.8 Hz, 1H), 2.96 (d, J=6.0 Hz, 2H), 2.61 (s, 1H), 2.61-2.50 (m, 2H), 2.38 (t, J=7.6 Hz, 2H), 1.65 (quint, J=7.2 Hz, 2H), 1.30-1.22 (m, 32H), 1.17 (d, J=6.0 Hz, 3H), 1.13 (d, J=6.0 Hz, 3H), 0.88 (t, J=6.8 Hz, 3H) and second eluting isomer (Example 6B, RT2: 2.53 min, 115 mg, 0.123 mmol, yield: 13.12%) as brown oil; LCMS (M+H)=934.4; Retention time (0.05% TFA)=4.03 min. HPLC Retention time (10 mM NH₄HCO₃)=15.31 min. ¹H NMR (400 MHz, CDCl₃) δ 7.99 (s, 1H), 7.24-7.20 (m, 5H), 7.16 (d, J=7.6 Hz, 2H), 7.12 (d, J=7.6 Hz, 3H), 6.40 (t, J=6.8 Hz, 1H), 5.91 (brs, 2H), 5.57 (dd, J=6.8, 4.0 Hz, 1H), 4.94 (quint, J=6.4 Hz, 1H), 4.30-4.22 (m, 2H), 4.13 (dd, J=10.8, 5.6 Hz, 1H), 3.60 (t, J=10.8 Hz, 1H), 2.99 (d, J=6.4 Hz, 2H), 2.69-2.55 (m, 3H), 2.40 (t, J=7.6 Hz, 2H), 1.72-1.68 (m, 2H), 1.28-1.22 (m, 32H), 1.14 (dd, J=8.0, 6.4 Hz, 6H), 0.88 (t, J=6.8 Hz, 3H).

### Example 7

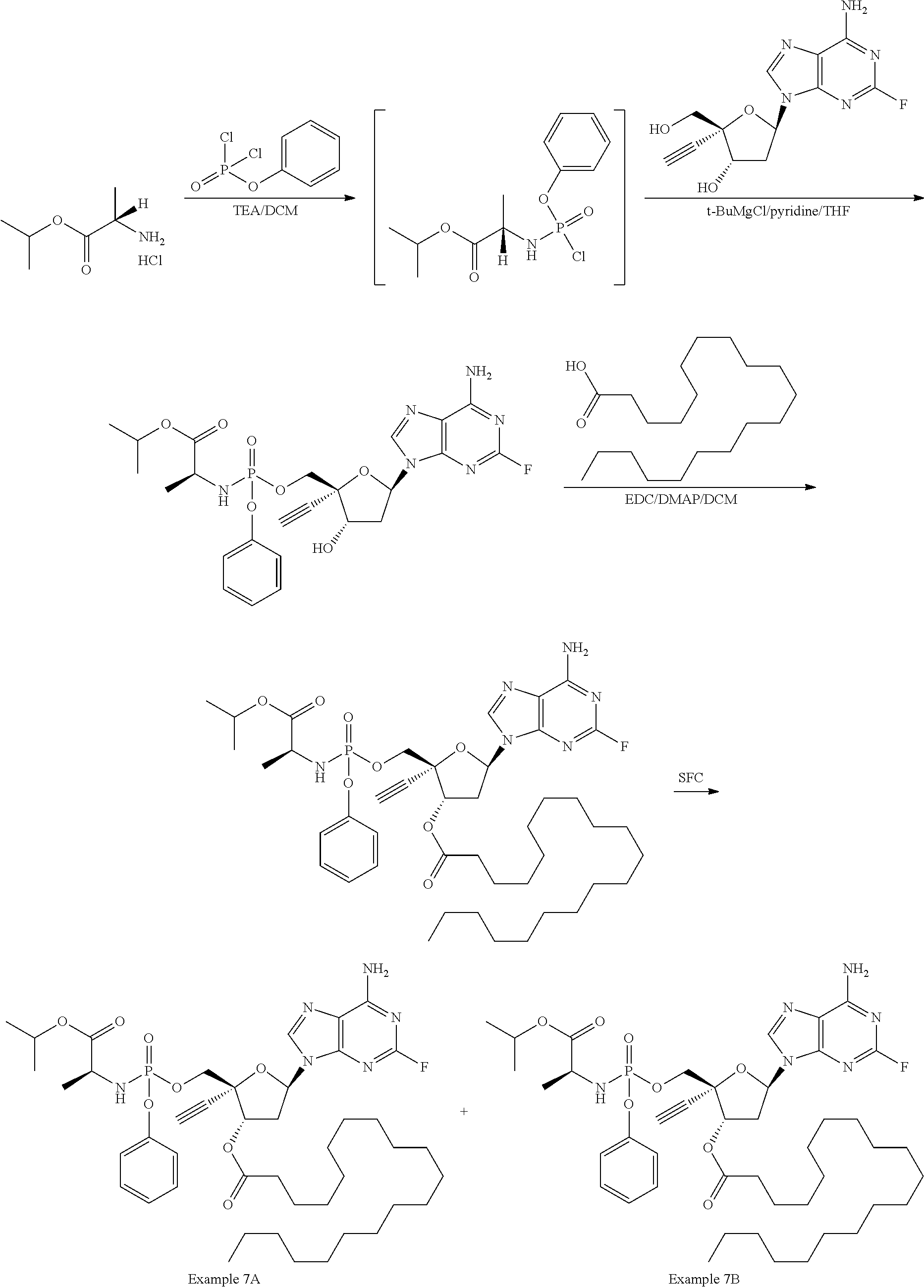

### Step 1: Isopropyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a cold (ice-water bath) mixture of isopropyl L-alaninate hydrochloride (2.095 g, 12.50 mmol), triethylamine (3.48 mL, 25.00 mmol) in DCM (60 mL) was added phenyl phosphorodichloridate (2.637 g, 12.50 mmol) in DCM (1 mL) dropwise under an atmosphere of nitrogen, then the reaction mixture was stirred at the same temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1466 mg, 5 mmol) in THF (60 mL) and pyridine (20 mL) was added tert-butylmagnesium chloride (12.50 mL, 12.50 mmol) dropwise under an atmosphere of nitrogen, then the mixture was stirred at room temperature for 30 min. The first prepared mixture was added dropwise to the second prepared mixture at room temperature and stirred for 2 h. LCMS showed the reaction was completed. The reaction mixture was filtered and the filtrate was concentrated, then the residue was diluted with EtOAc (200 mL). The organic layer was washed with water (40 mL), 1 N HCl (40 mL), brine (40 mL), dried over Na₂SO₄, filtered and concentrated. The residue was purified by silica gel chromatography (DCM/MeOH=20/1) to give isopropyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (510 mg, 0.907 mmol, 18.13% yield) as a white solid. LCMS (M+H)=563.0; Retention time (0.1% TFA)=1.48, 1.49 min.

### Step 2: (2R,3S,5R)-5-(6-Amino-2-fluoro-9H-purin-9-yl)-2-ethynyl-2-((((((S)-1-isopropoxy-1-oxopropan-2-yl)amino)(phenoxy)phosphoryl)oxy)methyl)tetrahydrofuran-3-yl icosanoate

To a solution of icosanoic acid (306 mg, 0.978 mmol) in DCM (20 mL) was added DMAP (543 mg, 4.44 mmol) and EDC (852 mg, 4.44 mmol), and the resulting mixture was stirred for 0.5 h at room temperature. Then isopropyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (500 mg, 0.889 mmol) was added, and the resulting mixture was stirred overnight at room temperature. LCMS showed the reaction was completed. The reaction mixture was diluted with DCM (100 mL), washed with water (30 mL), brine (30 mL), dried over Na₂SO₄, filtered and concentrated. The residue was purified by reverse phase chromatography (SepaFlash@ C18, BOSTON, 0-100%, 10 mM NH₄HCO₃/MeOH, 98%) to give (2R,3S,5R)-5-(6-amino-2-fluoro-9H-purin-9-yl)-2-ethynyl-2-((((((S)-1-isopropoxy-1-oxopropan-2-yl)amino)(phenoxy)phosphoryl)oxy)methyl)tetrahydrofuran-3-yl icosanoate (450 mg, 0.526 mmol, 59.13% yield) as a white solid. The diastereomers were separated with Prep-SFC (Instrument: SFC-80 (Thar, Waters), Column: AD 20*250 mm, 10 μm (Daicel), Column temperature: 40° C., Mobile phase: CO₂/MeOH (1.0% ammonia/methanol)=45/55, Flow rate: 80 g/min, Back pressure: 100 bar, Detection wavelength: 260 nm, Cycle time: 8.7 min to give first eluting isomer (Example 7A, RT1: 1.24 min, 160 mg, 0.187 mmol, yield: 21.00%) as white solid; LCMS (M+H)=857.3; Retention time (0.05% TFA)=3.76 min. HPLC Retention time (10 mM NH₄HCO₃)=13.23 min. ¹H NMR (400 MHz, CDCl₃) δ 8.04 (s, 1H), 7.32-7.28 (m, 2H), 7.25-7.20 (m, 2H), 7.13 (t, J=7.2 Hz, 1H), 6.45 (t, J=6.8 Hz, 1H), 6.11 (brs, 2H), 5.67 (dd, J=6.8, 4.0 Hz, 1H), 4.98 (quint, J=6.4 Hz, 1H), 4.42 (dd, J=6.0, 3.2 Hz, 2H), 4.02-3.93 (m, 1H), 3.86-3.80 (m, 1H), 2.75 (quint, J=7.2 Hz, 1H), 2.64 (s, 1H), 2.57 (ddd, J=14.0, 6.0, 4.0 Hz, 1H), 2.39 (t, J=7.6 Hz, 2H), 1.66 (quint, J=7.6 Hz, 2H), 1.30 (t, J=7.2 Hz, 3H), 1.32-1.22 (m, 32H), 1.20 (dd, J=8.0, 6.4 Hz, 6H), 0.88 (t, J=6.8 Hz, 3H) and second eluting isomer (Example 7B, RT2: 2.24 min, 211 mg, 0.243 mmol, yield: 27.4%) as white semi-solid; LCMS (M+H)=857.4; Retention time (0.05% TFA)=3.79 min. HPLC Retention time (10 mM NH₄HCO₃)=13.36 min. ¹H NMR (400 MHz, CDCl₃) δ 8.09 (s, 1H), 7.32-7.28 (m, 2H), 7.20 (d, J=8.8 Hz, 2H), 7.13 (t, J=7.2 Hz, 1H), 6.43 (t, J=6.8 Hz, 1H), 6.02 (brs, 2H), 5.65 (dd, J=6.8, 4.0 Hz, 1H), 5.01 (quint, J=6.4 Hz, 1H), 4.43 (dd, J=11.2, 6.8 Hz, 1H), 4.34 (dd, J=11.2, 6.0 Hz, 1H), 4.01 (dd, J=16.0, 8.8 Hz, 1H), 3.80 (t, J=10.4 Hz, 1H), 2.71 (quint, J=6.8 Hz, 1H), 2.67-2.60 (m, 2H), 2.40 (t, J=7.2 Hz, 2H), 1.66 (quint, J=7.6 Hz, 2H), 1.36 (d, J=6.8 Hz, 3H), 1.30-1.24 (m, 32H), 1.22 (d, J=6.4 Hz, 6H), 0.88 (t, J=6.8 Hz, 3H).

### Example 8

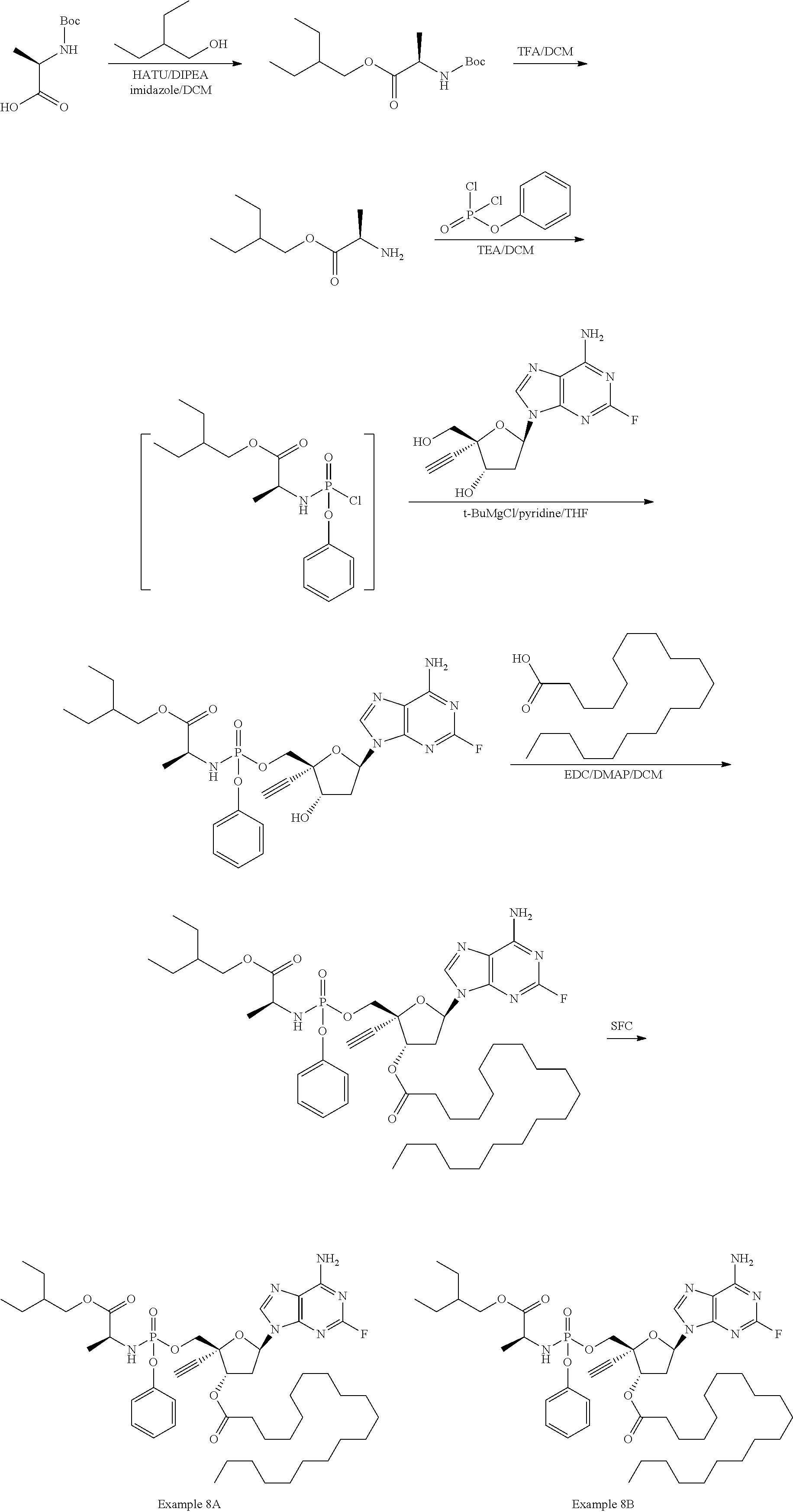

### Step 1: 2-Ethylbutyl (tert-butoxycarbonyl)-L-alaninate

To a mixture of 2-ethylbutan-1-ol (5.11 g, 50.0 mmol), (tert-butoxycarbonyl)-L-alanine (9.46 g, 50 mmol), and HATU (26.6 g, 70.0 mmol) in DCM (200 mL) were added 1H-imidazole (10.21 g, 150 mmol) and DIPEA (26.2 mL, 150 mmol). The reaction mixture was stirred at 25° C. overnight. LCMS showed the reaction was completed. The reaction was quenched with water (100 mL) and extracted with DCM (3×150 mL). The combined organic layers were washed with brine (100 mL), dried over Na₂SO₄, filtered and concentrated to afford 2-ethylbutyl (tert-butoxycarbonyl)-L-alaninate (13.67 g, 50.0 mmol, 100% yield) as a colorless oil which was used in the next step without further purification. LCMS (M+Na)=296.1; Retention time (0.1% TFA)=2.11 min.

### Step 2: 2-Ethylbutyl L-alaninate

To a solution of 2-ethylbutyl (tert-butoxycarbonyl)-L-alaninate (13.67 g, 50 mmol) in DCM (40 mL) was added TFA (20 mL, 260 mmol). The resulting reaction mixture was stirred at 25° C. for 3 h. LCMS showed the reaction was completed. The mixture was concentrated and the residue was diluted with NaHCO₃ (50 mL), and extracted with DCM (3×100 mL). The combined organic layers were washed with brine (80 mL), dried over Na₂SO₄, filtered and concentrated. The residue was purified by silica gel chromatography (DCM/MeOH=10/1) to give 2-ethylbutyl L-alaninate (8 g, 46.2 mmol, 92% yield) as a colorless oil. LCMS (M+H)=174.1; Retention time (0.1% TFA)=1.17 min.

### Step 3: 2-Ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To cold (ice-water bath) a mixture of 2-ethylbutyl L-alaninate (1.182 g, 6.82 mmol), triethylamine (1.188 mL, 8.52 mmol) in DCM (42 mL) was added phenyl phosphorodichloridate (1439 mg, 6.82 mmol) in DCM (1 mL) dropwise under an atmosphere of nitrogen, then the reaction mixture was stirred at the same temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1 g, 3.41 mmol) in THF (28 mL) and pyridine (14 mL) was added tert-butylmagnesium chloride (8.52 mL, 8.52 mmol) dropwise under an atmosphere of nitrogen, then the mixture was stirred at the same temperature for 30 min. Then, the first prepared mixture was added dropwise to the second prepared mixture at 0° C. The mixture was stirred at 10° C. for 2 h. LCMS showed the reaction was completed. The reaction mixture was filtered and concentrated. The residue was diluted with EtOAc (200 mL), washed with water (50 mL), brine (50 mL), dried over Na₂SO₄, filtered and concentrated. The residue was purified by Prep-HPLC to give 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (520 mg, 0.774 mmol, 22.70% yield) as a white solid. LCMS (M+H)=605.3; Retention time (0.05% TFA)=1.65 min.

### Step 4: (2R,3S,5R)-5-(6-Amino-2-fluoro-9H-purin-9-yl)-2-((((((S)-1-(2-ethylbutoxy)-1-oxopropan-2-yl)amino)(phenoxy)phosphoryl)oxy)methyl)-2-ethynyltetrahydrofuran-3-yl icosanoate

To a mixture of 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (520 mg, 0.860 mmol) and EDC (824 mg, 4.30 mmol) in DCM (10 mL) was added icosanoic acid (323 mg, 1.032 mmol). The resulting reaction mixture was stirred at room temperature overnight and concentrated to give the crude product. The residue was purified by flash chromatography (silica gel, 120 g, 0-100% DCM/MeOH) to give (2R,3S,5R)-5-(6-amino-2-fluoro-9H-purin-9-yl)-2-((((((S)-1-(2-ethylbutoxy)-1-oxopropan-2-yl)amino)(phenoxy)phosphoryl)oxy)methyl)-2-ethynyltetrahydrofuran-3-yl icosanoate (450 mg, 0.450 mmol, 52.4% yield) as a white semi-solid. LCMS (M+H)=900.1; Retention time (0.05% TFA)=4.32 min. The diastereomers were separated by Prep-SFC (Instrument: SFC-80 (Thar, Waters), Column: IG 20*250 mm, 10 μm (Daicel), Column temperature: 40° C., Mobile phase: CO₂/EtOH (1.0% ammonia/methanol)=35/65, Flow rate: 80 g/min, Back pressure: 100 bar, Detection wavelength: 260 nm, Cycle time: 6 min to give first eluting isomer (Example 8A, RT: 1.16 min, 142 mg, 0.155 mmol, yield: 43.5%) as light grey semi-solid; LCMS (M+H)=900.5; Retention time (0.05% TFA)=4.29 min. HPLC Retention time (10 mM NH₄HCO₃)=16.18 min. ¹H NMR (400 MHz, CDCl₃) δ 8.05 (s, 1H), 7.32-7.27 (m, 2H), 7.22 (d, J=8.0 Hz, 2H), 7.13 (t, J=7.6 Hz, 1H), 6.45 (t, J=6.8 Hz, 1H), 6.13 (brs, 2H), 5.67 (dd, J=6.8, 4.0 Hz, 1H), 4.42-4.39 (m, 2H), 4.09-3.98 (m, 3H), 3.85 (t, J=10.8 Hz, 1H), 2.75 (quint, J=6.8 Hz, 1H), 2.64 (s, 1H), 2.57 (ddd, J=13.6, 6.0, 4.0 Hz, 1H), 2.39 (t, J=7.6 Hz, 2H), 1.66 (quint, J=7.2 Hz, 2H), 1.49 (quint, J=6.0 Hz, 1H), 1.36-1.24 (m, 39H), 0.90-0.83 (m, 9H) and second eluting isomer (Example 8B, RT: 1.88 min, 105 mg, 0.117 mmol, yield: 32.8%) as light grey semi-solid; LCMS (M+H)=899.5; Retention time (0.05% TFA)=4.34 min. HPLC Retention time (10 mM NH₄HCO₃)=16.53 min. ¹H NMR (400 MHz, CDCl₃) δ 8.05 (s, 1H), 7.32-7.27 (m, 2H), 7.23-7.18 (m, 2H), 7.13 (t, J=7.6 Hz, 1H), 6.43 (t, J=6.8 Hz, 1H), 6.03 (brs, 2H), 5.65 (dd, J=6.8, 4.0 Hz, 1H), 4.43 (dd, J=11.2, 6.4 Hz, 1H), 4.35 (dd, J=11.2, 6.0 Hz, 1H), 4.11-4.05 (m, 2H), 3.99 (dd, J=10.8, 5.6 Hz, 1H), 3.84 (dd, J=11.2, 9.6 Hz, 1H), 2.72 (quint, J=7.2 Hz, 1H), 2.66-2.59 (m, 2H), 2.40 (t, J=7.6 Hz, 2H), 1.66 (quint, J=7.6 Hz, 2H), 1.50 (quint, J=6.0 Hz, 1H), 1.39 (d, J=7.2 Hz, 3H), 1.35-1.25 (m, 36H), 0.90-0.85 (m, 9H).

### Example 9

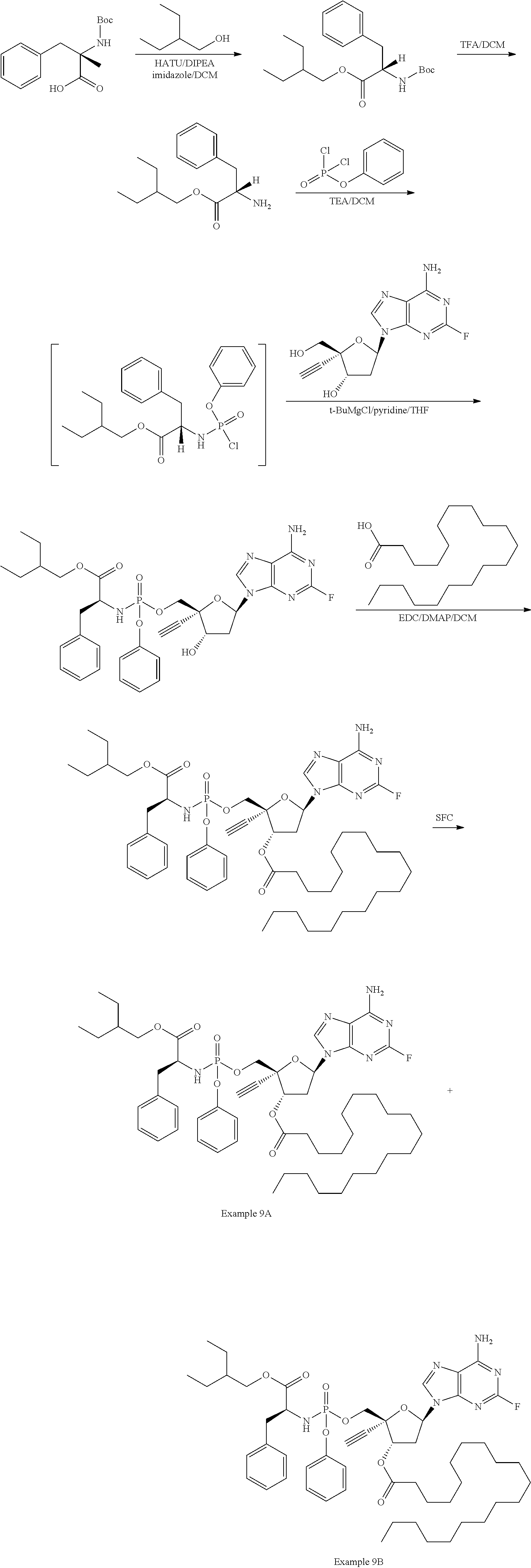

### Step 1: 2-Ethylbutyl (tert-butoxycarbonyl)-L-phenylalaninate

To a mixture of 2-ethylbutan-1-ol (5.11 g, 50.0 mmol), (tert-butoxycarbonyl)-L-phenylalanine (13.27 g, 50 mmol) and HATU (26.6 g, 70.0 mmol) in DCM (200 mL) were added 1H-imidazole (10.21 g, 150 mmol), DIPEA (26.2 mL, 150 mmol). The resulting reaction mixture was stirred at 25° C. overnight. LCMS showed the reaction was completed. The reaction was diluted with water (100 mL), organic layer separated and aqueous layer extracted with DCM (2×150 mL). The combined organic layers were washed with brine (100 mL), dried over Na₂SO₄, filtered and concentrated 2-ethylbutyl (tert-butoxycarbonyl)-L-phenylalaninate (17.45 g, 49.9 mmol, 100% yield) as colorless oil which was used in for the next step without further purification. LCMS (M+Na)=372.0; Retention time (0.1% TFA)=2.11 min.

### Step 2: 2-Ethylbutyl L-phenylalaninate

To a solution of 2-ethylbutyl (tert-butoxycarbonyl)-L-phenylalaninate (17.45 g, 49.9 mmol) in DCM (40 mL) was added 2,2,2-trifluoroacetic acid (20 mL, 260 mmol) and the mixture was stirred at 25° C. for 3 h. LCMS showed the reaction was completed. The mixture was concentrated and the residue was purified by silica gel chromatography (DCM/MeOH=10/1) to give 2-ethylbutyl L-phenylalaninate (9.5 g, 38.1 mmol, 76.35% yield) as a colorless oil. LCMS (M+H)=250.2; Retention time (0.01% TFA)=1.46 min.

### Step 3: 2-Ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a solution of 2-ethylbutyl L-phenylalaninate (4.25 g, 17.05 mmol), triethylamine (2.376 mL, 17.05 mmol) in DCM (50 mL) was added phenyl phosphorodichloridate (2.55 mL, 17.05 mmol) in DCM (5 mL) dropwise under an atmosphere of nitrogen at 0° C., then the reaction mixture was stirred at 0° C. for 1 h. To a solution of (2R,3S,5R)-5-(6-amino-2-fluoro-9H-purin-9-yl)-2-ethynyl-2-(hydroxymethyl)tetrahydrofuran-3-ol (2 g, 6.82 mmol) in THF (20 mL) and pyridine (10 mL) was added tert-butylmagnesium chloride (17.05 mL, 17.05 mmol) dropwise under an atmosphere of nitrogen at 0° C. Then, the mixture was stirred at the same temperature for 30 min. The first prepared mixture was added dropwise to the second prepared mixture at 0° C. and stirred at 10° C. for 2 h. LCMS showed the reaction was completed. The reaction mixture was filtered and concentrated. The residue was diluted with DCM (200 mL), washed with water (40 mL), dried over Na₂SO₄, filtered and concentrated. The residue was purified by reverse phase chromatography (SepaFlash@ C18, BOSTON, 0-100% CH₃CN/10 mM NH₄HCO₃H2O) to give 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (850 mg, 1.073 mmol, 15.73% yield) as a white solid. LCMS (M+H)=681.0; Retention time (10 mM NH₄HCO₃)=1.83 min.

### Step 4: (2R,3S,5R)-5-(6-Amino-2-fluoro-9H-purin-9-yl)-2-((((((S)-1-(2-ethylbutoxy)-1-oxo-3-phenylpropan-2-yl)amino)(phenoxy)phosphoryl)oxy)methyl)-2-ethynyltetrahydrofuran-3-yl icosanoate

To a solution of icosanoic acid (303 mg, 0.970 mmol) in DCM (50 mL) was added DMAP (538 mg, 4.41 mmol) and EDC (845 mg, 4.41 mmol) and the resulting mixture was stirred at room temperature for 0.5 h. Then 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (600 mg, 0.881 mmol) was added, the resulting mixture was stirred overnight at room temperature. The LCMS showed the reaction was completed. The reaction mixture was diluted with DCM (100 mL) and washed with water (30 mL), brine (30 mL), dried over Na₂SO₄, filtered and concentrated. The residue was purified by reverse phase chromatography (SepaFlash@ C18, BOSTON, 0-100% MeOH/10 mM NH₄HCO₃H2O) to give (2R,3S,5R)-5-(6-amino-2-fluoro-9H-purin-9-yl)-2-((((((S)-1-(2-ethylbutoxy)-1-oxo-3-phenylpropan-2-yl)amino)(phenoxy)phosphoryl)oxy)methyl)-2-ethynyltetrahydrofuran-3-yl icosanoate (600 mg, 0.615 mmol, 69.81% yield) as a colorless oil. The diastereomers were separated with Prep-SFC (Instrument: SFC-80 (Thar, Waters), Column: IG 20*250 mm, 10 μm (Daicel), Column temperature: 40° C., Mobile phase: CO₂/EtOH (1.0% ammonia/methanol)=35/65, Flow rate: 80 g/min, Back pressure: 100 bar, Detection wavelength: 260 nm, Cycle time: 9.6 min to give first eluting isomer (Example 9A, RT: 1.58 min, 130 mg, 0.133 mmol, yield: 15.12%) as light grey solid; LCMS (M+H)=976.3; Retention time (0.05% TFA)=4.70 min. HPLC Retention time (10 mM NH₄HCO₃)=17.22 min. ¹H NMR (400 MHz, CDCl₃) δ 8.01 (s, 1H), 7.25-7.10 (m, 8H), 7.04 (dd, J=8.0, 2.0 Hz, 2H), 6.43 (t, J=6.8 Hz, 1H), 6.06 (brs, 2H), 5.59 (dd, J=6.8, 4.0 Hz, 1H), 4.29 (dd, J=10.8, 6.4 Hz, 1H), 4.27-4.21 (m, 1H), 4.11 (dd, J=10.8, 6.0 Hz, 1H), 3.99 (dd, J=10.8, 5.6 Hz, 1H), 3.92 (dd, J=10.8, 6.0 Hz, 1H), 3.65 (t, J=10.8 Hz, 1H), 2.97 (t, J=6.8 Hz, 2H), 2.65-2.58 (m, 2H), 2.51 (ddd, J=13.6, 6.0, 3.6 Hz, 1H), 2.38 (t, J=7.6 Hz, 2H), 1.65 (quint, J=7.6 Hz, 2H), 1.43 (quint, J=6.4 Hz, 1H), 1.32-1.22 (m, 36H), 0.88 (t, J=6.8 Hz, 3H), 0.83 (t, J=7.6 Hz, 6H) and second eluting isomer (Example 9B, RT: 2.32 min, 170 mg, 0.172 mmol, yield: 19.50%) as colorless oil; LCMS (M+H)=975.3; Retention time (0.05% TFA)=4.73 min. HPLC Retention time (10 mM NH₄HCO₃)=19.12 min. ¹H NMR (400 MHz, CDCl₃) δ 7.99 (s, 1H), 7.26-7.21 (m, 4H), 7.20-7.14 (m, 3H), 7.11 (dd, J=6.8, 1.6 Hz, 3H), 6.40 (t, J=6.8 Hz, 1H), 6.20 (brs, 2H), 5.57 (dd, J=6.8, 4.4 Hz, 1H), 4.39-4.29 (m, 1H), 4.24 (dd, J=10.8, 6.4 Hz, 1H), 4.13 (dd, J=11.2, 5.6 Hz, 1H), 3.95 (dd, J=6.0, 2.0 Hz, 2H), 3.74 (t, J=10.8 Hz, 1H), 3.06-2.95 (m, 2H), 2.67-2.54 (m, 3H), 2.40 (t, J=7.2 Hz, 2H), 1.67 (quint, J=7.6 Hz, 2H), 1.42 (quint, J=6.4 Hz, 1H), 1.32-1.21 (m, 36H), 0.88 (t, J=6.8 Hz, 3H), 0.83 (t, J=7.6 Hz, 6H).

### Example 10

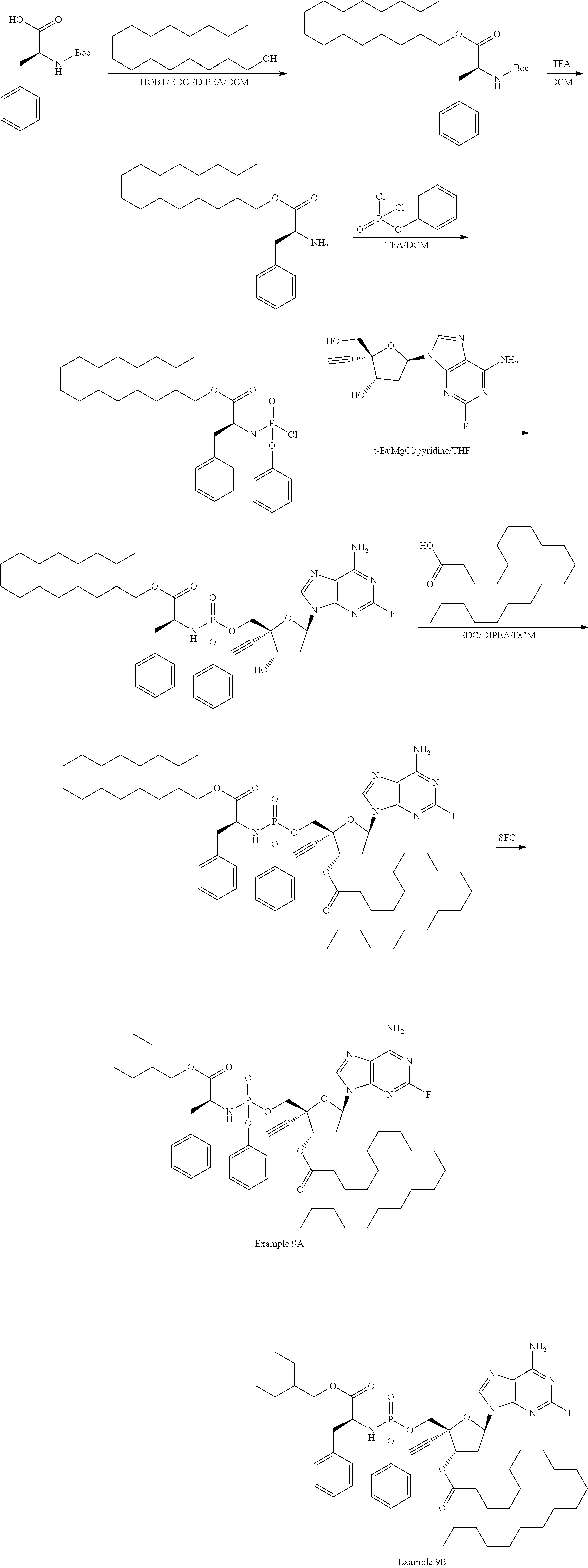

### Step 1: Hexadecyl (tert-butoxycarbonyl)-L-phenylalaninate

To a mixture of hexadecan-1-ol (15 g, 61.9 mmol), (tert-butoxycarbonyl)-L-phenylalanine (16.41 g, 61.9 mmol), 1H-imidazole (12.64 g, 186 mmol) and HATU (35.3 g, 93 mmol) in DCM (200 mL) was added DIPEA (32.4 mL, 186 mmol) and stirred at room temperature for 16 h. TLC showed reaction was completed. The reaction mixture was washed with water, dried over Na₂SO₄, filtered and concentrated to give crude product. The crude product was purified by flash chromatography (pet. ether:EtOAc=20:1) to give hexadecyl (tert-butoxycarbonyl)-L-phenylalaninate (23 g, 47.0 mmol, 76% yield) as a white solid.

### Step 2: Hexadecyl L-phenylalaninate

To a solution of hexadecyl (tert-butoxycarbonyl)-L-phenylalaninate (23 g, 47.0 mmol) in DCM (500 mL) was added trifluoroacetic acid (100 mL) and stirred at room temperature for 12 h. TLC showed the reaction was completed. The reaction mixture was concentrated and the pH of the residue was adjusted to 8 with NaHCO₃ (aq) and extracted twice with EtOAc (500 mL). The combined organic phases were washed with water (300 mL), brine (300 mL), dried over Na₂SO₄, filtered and concentrated to give crude product. The crude product was purified by flash chromatography (pet. ether:EtOAc=5:1) to give hexadecyl L-phenylalaninate (18 g, 46.2 mmol, 98% yield) as a white solid.

### Step 3: Hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold (ice-water bath) solution of 1-((L-phenylalanyl)oxy)hexadecan-7-ylium (5301 mg, 13.64 mmol), triethylamine (1.901 mL, 13.64 mmol) in DCM (42 mL) was added phenyl phosphorodichloridate (2878 mg, 13.64 mmol) in DCM (1 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (2000 mg, 6.82 mmol) in THF (28 mL)/pyridine (14 mL) was added tert-butylmagnesium chloride (17.05 mL, 17.05 mmol) dropwise under an atmosphere of nitrogen, then the mixture was stirred at room temperature for 30 min. The first prepared mixture was added dropwise to the second prepared mixture by at room temperature and stirred for 16 h. LCMS indicated completion of reaction. The reaction mixture was filtered and concentrated. The residue was diluted with EtOAc (150 mL) and washed with water (80 mL) twice. The organic phase was dried over Na₂SO₄, filtered and concentrated to give crude product. The crude product was purified by Prep-HPLC to give hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (1.3 g, 1.472 mmol, 21.59% yield) as a white solid. LCMS (M+H)=821.0; Retention time (10 mM NH₄HCO₃)=3.162 min.

### Step 4: (2R,3S,5R)-5-(6-Amino-2-fluoro-9H-purin-9-yl)-2-ethynyl-2-((((((S)-1-(hexadecyloxy)-1-oxo-3-phenylpropan-2-yl)amino)(phenoxy)phosphoryl)oxy)methyl)tetrahydrofuran-3-yl icosanoate

To a solution of icosanoic acid (293 mg, 0.938 mmol) in DCM (20 mL) was added DMAP (521 mg, 4.26 mmol) and EDC (817 mg, 4.26 mmol) and the resulting mixture was stirred for 0.5 h at room temperature. Then, hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (700 mg, 0.853 mmol) was added and the resulting mixture was stirred overnight at room temperature. TLC (DCM:MeOH=10:1, Rf=0.8) showed the reaction was completed. The reaction mixture was quenched with water (10 mL) and extracted with DCM (20 mL×3). The combined organic phases were washed with brine (30 mL), dried over Na₂SO₄ and concentrated under vacuum. The crude was purified by prep-TLC (DCM:CH₃OH=10:1, Rf=0.7) to give (2R,3S,5R)-5-(6-amino-2-fluoro-9H-purin-9-yl)-2-ethynyl-2-((((((S)-1-(hexadecyloxy)-1-oxo-3-phenylpropan-2-yl)amino)(phenoxy)phosphoryl)oxy)methyl)tetrahydrofuran-3-yl icosanoate (290 mg, 0.260 mmol, 30.5% yield). The diastereomers were separated with Prep-SFC (Instrument: SFC-80 (Thar, Waters); Column: OZ 20×250 mm, 10 μm (Daicel); Column temperature: 40° C.; Mobile phase: CO₂/MEOH (1.0% ammonia/methanol)=40/60; Flow rate: 80 g/min; Back pressure: 100 bar; Detection wavelength: 260 nm; Cycle time: 9.5 min; Sample solution: 290 mg dissolved in 17 ml methanol; Injection volume: 3 mL) to obtain first eluting isomer (Example 10A, RT: 1.88 min, 90 mg, 0.078 mmol, 29.9% yield) as white solid; HPLC: Retention time (0.1% NH₄HCO₃)=38.538 min; ¹H NMR (400 MHz, CDCl₃) δ 8.01 (s, 1H), 7.27 (d, J=6.7 Hz, 1H), 7.25-7.20 (m, 2H), 7.20-7.15 (m, 3H), 7.15-7.09 (m, 2H), 7.05-7.01 (m, 2H), 6.42 (t, J=6.8 Hz, 1H), 6.14 (s, 2H), 5.58 (dd, J=6.6, 3.8 Hz, 1H), 4.29 (dd, J=11.0, 6.6 Hz, 1H), 4.26-4.18 (m, 1H), 4.10 (dd, J=11.0, 5.8 Hz, 1H), 4.01 (t, J=6.1 Hz, 2H), 3.69 (t, J=7.0 Hz, 1H), 2.96 (d, J=6.2 Hz, 2H), 2.61 (s, 1H), 2.57 (dd, J=12.9, 5.9 Hz, 1H), 2.54-2.47 (m, 1H), 2.38 (t, J=7.5 Hz, 2H), 1.76 (s, 6H), 1.69-1.60 (m, 2H), 1.24 (d, J=7.6 Hz, 54H), 0.87 (t, J=6.7 Hz, 6H) and second eluting isomer (Example 10B, RT: 2.93 min, 96 mg, 0.084 mmol, 32.3% yield) as white solid; HPLC: Retention time (0.1% NH₄HCO₃)=39.401 min; ¹H NMR (400 MHz, CDCl₃) δ 8.01 (s, 1H), 7.25-7.18 (m, 5H), 7.15 (d, J=8.5 Hz, 2H), 7.11 (t, J=5.7 Hz, 3H), 6.39 (t, J=6.7 Hz, 1H), 5.99 (s, 2H), 5.59-5.54 (m, 1H), 4.31 (ddd, J=16.5, 10.2, 6.3 Hz, 1H), 4.25 (dd, J=11.0, 6.2 Hz, 1H), 4.13 (dd, J=11.0, 5.7 Hz, 1H), 4.02 (t, J=6.7 Hz, 2H), 3.63 (t, J=8.6 Hz, 1H), 3.00 (d, J=6.4 Hz, 2H), 2.63-2.54 (m, 3H), 2.40 (t, J=7.6 Hz, 2H), 1.79-1.62 (m, 6H), 1.25 (s, 56H), 0.88 (t, J=6.8 Hz, 6H).

### Example 11

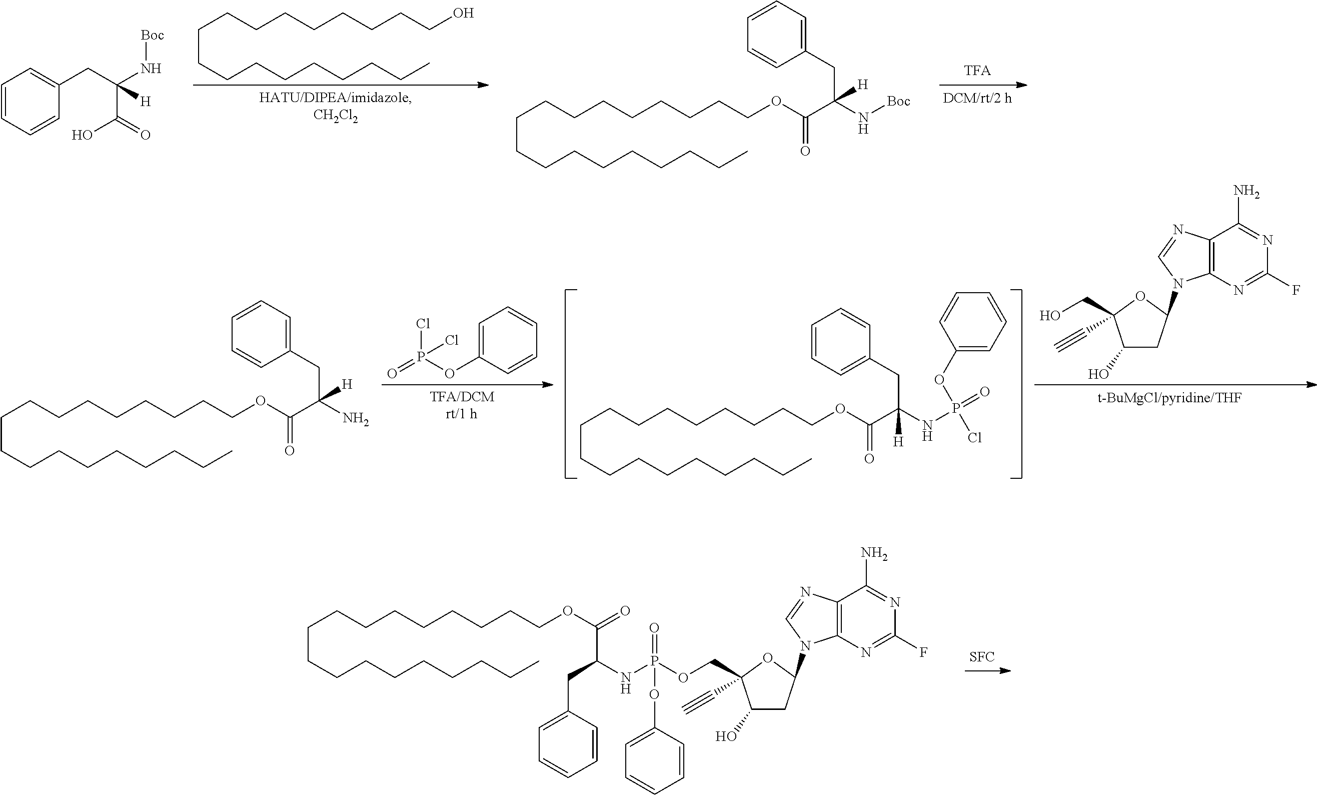

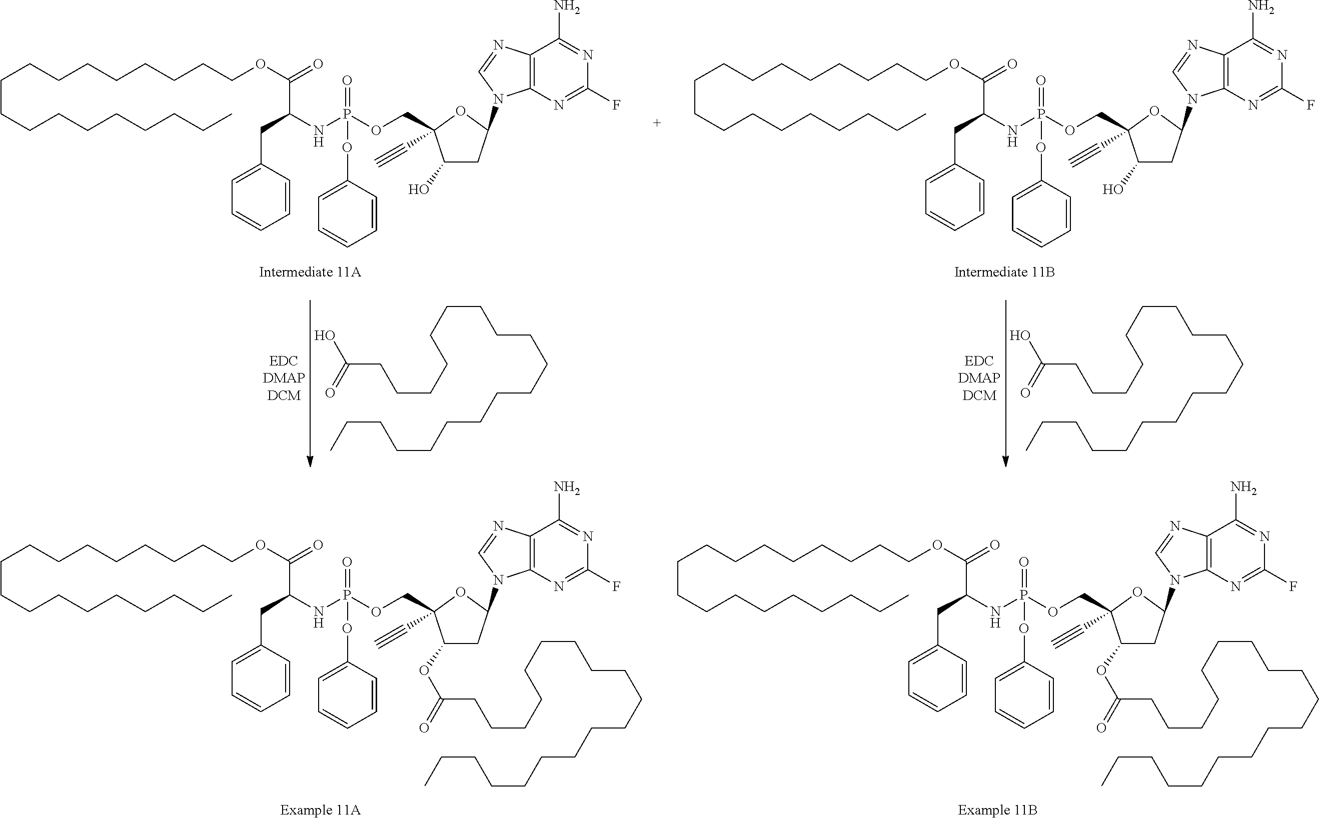

### Step 1: Octadecyl (tert-butoxycarbonyl)phenylalaninate

To a solution of octadecan-1-ol (32.5 g, 120 mmol), (tert-butoxycarbonyl)-L-phenylalanine (29 g, 109 mmol), and HATU (62.3 g, 164 mmol) in DCM (100 mL) was added 1H-imidazole (22.32 g, 328 mmol) and DIPEA (57.3 mL, 328 mmol). The resulting mixture was stirred at 25° C. overnight. TLC (Pet. ether:EtOAc=10:1, Rf=0.5) showed completion of reaction. The reaction was quenched with water (200 mL) and DCM (100 mL). Then, the organic layer was separated and the aqueous layer was extracted with DCM (2×50 mL). The combined organic layers were washed with brine (200 mL), dried over Na₂SO₄, filtered and concentrated to give crude product. The residue was purified by combiflash (silica gel column 330 g, hexane:ethyl acetate=20:1). The appropriate fractions containing desired product were combined and concentrated in vacuum to give octadecyl (tert-butoxycarbonyl)phenylalaninate (32 g, 61.8 mmol, 56.5% yield) as yellow solid.

### Step 2: Octadecyl phenylalaninate

To a solution of octadecyl (tert-butoxycarbonyl)phenylalaninate (32 g, 61.8 mmol) in DCM (400 mL) was added TFA (28.6 mL, 371 mmol). The resulting mixture was stirred at 25° C. for 2 h. TLC (pet. ether:EtOAc=10:1, Rf=0.1) showed the reaction was completed. The reaction was quenched with 1N NaHCO₃ (200 mL) and DCM (100 mL), then the organic layer was separated. The aqueous layer was extracted with DCM (2×100 mL). The combined organic layers were washed with brine (200 mL), dried over Na₂SO₄ and concentrated to give crude product octadecyl phenylalaninate (19 g, 45.5 mmol, 73.6% yield) which was used in the next step without purification.

### Step 3: Octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)phenylalaninate

To a solution of octadecyl L-phenylalaninate (5.70 g, 13.64 mmol) and triethylamine (1.901 mL, 13.64 mmol) in DCM (80 mL) was added phenyl phosphorodichloridate (2.038 mL, 13.64 mmol) in DCM (80 mL) dropwise under an atmosphere of nitrogen at about 5° C. Then, the reaction mixture was stirred at room temperature for 1 h. To a solution of (2R,3S,5R)-5-(6-amino-2-fluoro-9H-purin-9-yl)-2-ethynyl-2-(hydroxymethyl)tetrahydrofuran-3-ol (2 g, 6.82 mmol) in THF (40 mL)/pyridine (20 mL) was added tert-butylmagnesium chloride (17.05 mL, 17.05 mmol) dropwise under an atmosphere of nitrogen at 0° C. Then, the mixture was stirred at room temperature for 30 min. The first prepared mixture was added dropwise to the second prepared mixture at room temperature and stirred for 2 h. LCMS indicated completion of reaction. The mixture was filtered and concentrated to afford yellow solid. Then, the solid was dissolved in EtOAc (40 mL) and partitioned with HCl (0.05 N, 20 mL). The layers were separated, and the aqueous layer was extracted with EtOAc (40 mL). The combined organic phases were washed with brine, dried over Na₂SO₄, filtered and concentrated to afford the crude product. The residue was purified by reverse phase chromatography (SepaFlash@ C18, BOSTON, 0-100% CH₃CN/10 mM NH₄HCO₃H₂O) to give octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)phenylalaninate (1.2 g, 1.354 mmol, 19.85% yield) as yellow solid. LCMS (M+H)=849.4; Retention time (0.1% TFA)=3.568 min. The diastereomers were separated by Prep-SFC (Instrument: SFC-150 (Waters), Column: IH 20×250 mm, 10 μm (Daicel), Column temperature: 35° C., Mobile phase: CO₂/MeOH (0.2% ammonia/methanol)=60/40, Flow rate: 100 g/min, Back pressure: 100 bar, Detection wavelength: 214 nm, Cycle time: 4 min, Sample solution: 1,900 mg dissolved in 100 mL methanol, Injection volume: 3 mL) to give first eluting isomer (Intermediate 11A, RT: 1.76 min, 800 mg, 0.908 mmol, 38.5% yield); LCMS (M+H)=849.4; Retention time (0.1% TFA)=2.874 min. HPLC: Retention time (0.1% NH₄HCO₃): 11.004 min; ¹H NMR (400 MHz, CDCl₃) δ 7.92 (s, 1H), 7.25-7.17 (m, 5H), 7.14 (d, J=7.3 Hz, 1H), 7.12-7.08 (m, 4H), 6.31 (dd, J=7.3, 4.3 Hz, 1H), 6.10 (s, 2H), 4.71 (t, J=7.6 Hz, 1H), 4.26 (dd, J=11.2, 7.7 Hz, 2H), 4.20 (dd, J=11.3, 8.8 Hz, 1H), 4.03 (t, J=6.7 Hz, 2H), 3.88 (s, 1H), 2.99 (d, J=6.2 Hz, 2H), 2.76-2.63 (m, 3H), 1.86 (s, 2H), 1.24 (d, J=9.0 Hz, 30H), 0.88 (t, J=6.8 Hz, 3H) and second eluting isomer (Intermediate 11B, RT: 3.21 min, 650 mg, 0.766 mmol, 32.5% yield) as white solid; LCMS (M+H)=849.5; Retention time (0.1% TFA)=2.839 min. HPLC: Retention time (0.1% NH₄HCO₃): 10.931 min; ¹H NMR (400 MHz, CDCl₃) δ 7.78 (s, 1H), 7.30-7.26 (m, 2H), 7.23 (dd, J=8.4, 4.6 Hz, 3H), 7.15 (d, J=7.8 Hz, 3H), 7.06 (dd, J=7.5, 1.7 Hz, 2H), 6.32 (dd, J=7.4, 4.2 Hz, 1H), 6.24 (s, 2H), 4.64 (t, J=7.6 Hz, 1H), 4.23-4.15 (m, 2H), 4.02 (t, J=6.7 Hz, 2H), 3.97 (dd, J=11.5, 8.4 Hz, 1H), 3.73 (t, J=10.8 Hz, 1H), 3.04-2.92 (m, 2H), 2.70 (s, 1H), 2.66 (dt, J=13.7, 7.8 Hz, 1H), 2.57 (ddd, J=13.6, 7.1, 4.2 Hz, 1H), 1.89 (s, 2H), 1.24 (d, J=4.9 Hz, 30H), 0.87 (t, J=6.8 Hz, 3H).

### Step 5: (2R,3S,5R)-5-(6-amino-2-fluoro-9H-purin-9-yl)-2-ethynyl-2-((((((S)-1-(octadecyloxy)-1-oxo-3-phenylpropan-2-yl)amino)(phenoxy)phosphoryl)oxy)methyl)tetrahydrofuran-3-yl icosanoate

To a solution of icosanoic acid (101 mg, 0.324 mmol) in DCM (10 mL) was added DMAP (180 mg, 1.472 mmol) and EDC (282 mg, 1.472 mmol) and the resulting mixture was stirred for 0.5 h at room temperature. Then, octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 11A, 250 mg, 0.294 mmol) was added and the resulting mixture was stirred overnight at room temperature. TLC showed the reaction was completed. The reaction mixture was diluted with DCM (100 mL), washed with water, brine, dried over Na₂SO₄, filtered and concentrated. The residue was purified by prep-TLC (DCM/MeOH=10/1) to give (2R,3S,5R)-5-(6-amino-2-fluoro-9H-purin-9-yl)-2-ethynyl-2-((((((S)-1-(octadecyloxy)-1-oxo-3-phenylpropan-2-yl)amino)(phenoxy)phosphoryl)oxy)methyl)tetrahydrofuran-3-yl icosanoate (Example 11A, 145 mg, 0.127 mmol, 43.1% yield) as white solid. HPLC: Retention time (10 mM NH₄HCO₃)=60.25 min; ¹H NMR (400 MHz, CDCl₃) δ 7.99 (s, 1H), 7.26-7.18 (m, 5H), 7.15 (d, J=8.4 Hz, 2H), 7.13-7.08 (m, 3H), 6.39 (t, J=6.8 Hz, 1H), 6.16 (brs, 2H), 5.56 (dd, J=6.4, 4.4 Hz, 1H), 4.36-4.29 (m, 1H), 4.25 (dd, J=10.8, 6.4 Hz, 1H), 4.13 (dd, J=10.8, 5.6 Hz, 1H), 4.02 (t, J=6.8 Hz, 2H), 3.73 (t, J=10.8 Hz, 1H), 3.02-2.98 (m, 2H), 2.65-2.55 (m, 3H), 2.40 (t, J=7.2 Hz, 2H), 1.67 (quint, J=6.8 Hz, 2H), 1.52 (quint, J=6.0 Hz, 2H), 1.30-1.22 (m, 62H), 0.90-0.85 (m, 6H).

To a solution of icosanoic acid (101 mg, 0.324 mmol) in DCM (10 mL) was added DMAP (180 mg, 1.472 mmol) and EDC (282 mg, 1.472 mmol). The resulting mixture was stirred for 0.5 h at room temperature. Then, octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 11B, 250 mg, 0.294 mmol) was added, and the resulting mixture was stirred overnight at room temperature. The LCMS showed the reaction was completed. The reaction mixture was diluted with water (10 mL) and extracted with DCM (15 mL×3). The combined organic phases were washed with brine (20 mL), dried over Na₂SO₄ and concentrated under vacuum. The crude was purified by prep-TLC (DCM:CH₃OH=10:1, Rf=0.7) to give (2R,3S,5R)-5-(6-amino-2-fluoro-9H-purin-9-yl)-2-ethynyl-2-((((((S)-1-(octadecyloxy)-1-oxo-3-phenylpropan-2-yl)amino)(phenoxy)phosphoryl)oxy)methyl)tetrahydrofuran-3-yl icosanoate (Example 11B, 115 mg, 0.101 mmol, 34.2% yield). HPLC: Retention time (0.1% NH₄HCO₃)=63.275 min. ¹H NMR (400 MHz, CDCl₃) δ 8.03 (s, 1H), 7.27 (d, J=7.2 Hz, 1H), 7.25-7.20 (m, 2H), 7.18 (d, J=5.3 Hz, 2H), 7.15 (d, J=8.2 Hz, 2H), 7.12 (t, J=7.4 Hz, 1H), 7.06-7.01 (m, 2H), 6.43 (t, J=6.8 Hz, 1H), 6.13 (s, 2H), 5.58 (dd, J=6.7, 3.8 Hz, 1H), 4.29 (dd, J=11.0, 6.6 Hz, 1H), 4.26-4.18 (m, 1H), 4.11 (dd, J=11.0, 5.8 Hz, 1H), 4.01 (td, J=6.8, 1.6 Hz, 2H), 3.68 (t, J=10.7 Hz, 1H), 2.96 (d, J=6.3 Hz, 2H), 2.64-2.47 (m, 3H), 2.38 (t, J=7.6 Hz, 2H), 1.82 (s, 2H), 1.69-1.60 (m, 2H), 1.30-1.21 (m, 62H), 0.88 (t, J=6.8 Hz, 6H).

### Example 12

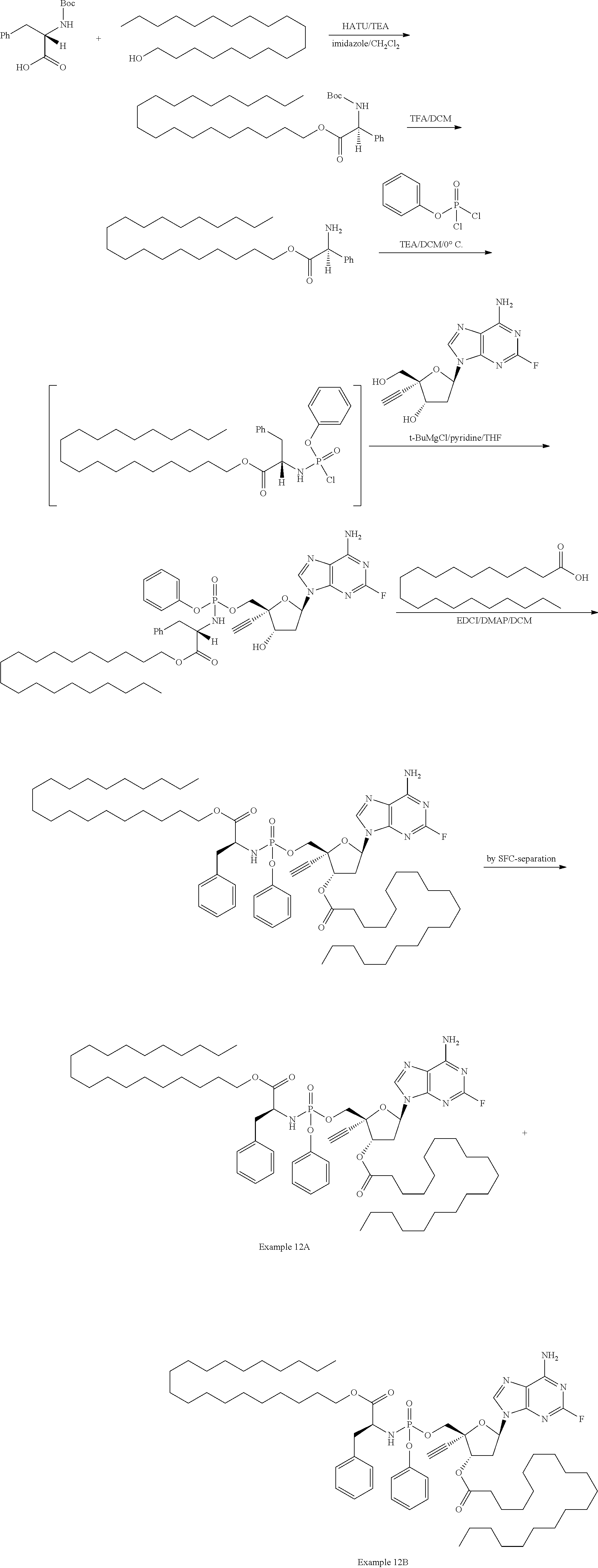

### Step 1: Icosyl (tert-butoxycarbonyl)-L-phenylalaninate

A mixture of icosan-1-ol (33.8 g, 113 mmol), (tert-butoxycarbonyl)-L-phenylalanine (25 g, 94 mmol), imidazole (19.25 g, 283 mmol), DIPEA (49.4 mL, 283 mmol) and HATU (53.7 g, 141 mmol) in DCM (200 mL) was stirred at 25° C. for 16 h. TLC showed the presence of new product. Water (200 mL) was added and the mixture was extracted with DCM (150 mL×3). The combined organic phases were washed with brine, dried over Na₂SO₄, filtered and concentrated. The residue was purified by silica gel chromatography (pet. ether:EtOAc=30:1) to afford icosyl (tert-butoxycarbonyl)-L-phenylalaninate (20 g, 10.99 mmol, 11.67% yield) as white solid. ¹H NMR (400 MHz, CDCl₃) δ 7.3-7.28 (m, 1H), 7.26-7.11 (m, 4H), 4.99 (d, J=7.4 Hz, 1H), 4.57 (d, J=7.3 Hz, 1H), 4.12-4.02 (m, 2H), 3.49 (s, 1H), 3.17-2.99 (m, 2H), 1.42 (s, 8H), 1.26 (s, 36H), 0.88 (t, J=6.8 Hz, 3H).

### Step 2: Icosyl L-phenylalaninate

To a solution of icosyl (tert-butoxycarbonyl)-L-phenylalaninate (30 g, 55.0 mmol) in DCM (200 mL) was added TFA (42.3 mL, 550 mmol) at 0° C. After 5 min, the mixture was warmed to room temperature and stirred for 2 h. TLC showed starting material was consumed completely. The pH of the reaction mixture was adjusted to 7-8 with sat. Na₂CO₃ solution and extracted with DCM (300 mL×3). The combined organic phases were washed with brine, dried over Na₂SO₄, filtered and concentrated to give the residue. The residue was purified by silica gel chromatography (DCM:MeOH=20:1) to give icosyl L-phenylalaninate (25 g, 50.5 mmol, 92% yield). ¹H NMR (400 MHz, DMSO) δ 7.34-7.08 (m, 5H), 4.00-3.88 (m, 2H), 3.55 (t, J=6.8 Hz, 1H), 2.92-2.73 (m, 2H), 1.91 (s, 2H), 1.52-1.39 (m, 2H), 1.23 (s, 35H), 0.85 (t, J=6.8 Hz, 3H).

### Step 3: Icosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold (ice-water bath) solution of icosyl L-phenylalaninate (7.60 g, 17.05 mmol), triethylamine (2.376 mL, 17.05 mmol) in DCM (48 mL) was added phenyl phosphorodichloridate (3.60 g, 17.05 mmol) in DCM (1 mL) dropwise under an atmosphere of nitrogen in ice water, then the reaction mixture was stirred at room temperature for 1 h. To a solution of (2R,3S,5R)-5-(6-amino-2-fluoro-9H-purin-9-yl)-2-ethynyl-2-(hydroxymethyl)tetrahydrofuran-3-ol (2 g, 6.82 mmol) in THF (24 mL)/pyridine (12 mL) was added tert-butylmagnesium chloride (17.05 mL, 17.05 mmol) dropwise under an atmosphere of nitrogen at 0° C., and then the mixture was warmed to room temperature and stirred for 30 min. The first prepared mixture was added dropwise to the second prepared mixture at room temperature and stirred for 2 h. LCMS indicated the completion of reaction. The reaction mixture was filtered, concentrated, and then the residue was diluted with DCM. The mixture was added water and extracted. Then, the organic layer was washed with 0.5 N HCl three times and brine. The organic phase was dried over Na₂SO₄, filtered and concentrated. The residue was purified by flash chromatography (0-5% MeOH/DCM) to give icosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (800 mg, 0.867 mmol, 12.71% yield). LCMS (M+H)=877.7, Retention time (0.1% TFA)=3.82 min.

### Step 4: (2R,3S,5R)-5-(6-Amino-2-fluoro-9H-purin-9-yl)-2-ethynyl-2-((((((S)-1-(icosyloxy)-1-oxo-3-phenylpropan-2-yl)amino)(phenoxy)phosphoryl)oxy)methyl)tetrahydrofuran-3-yl icosanoate

To a solution of icosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (1200 mg, 1.368 mmol) in DCM (10 mL) was added DMAP (167 mg, 1.368 mmol), EDC (1311 mg, 6.84 mmol), DIEA (0.717 mL, 4.10 mmol) and icosanoic acid (428 mg, 1.368 mmol). The resulting mixture was stirred overnight at room temperature. TLC showed the presence of new compound. The reaction mixture was diluted with water and extracted with DCM (40 mL×3). The combined organic phases were washed with brine, dried over Na₂SO₄, filtered and concentrated to give the residue. The residue was purified by prep-TLC (DCM:MeOH=10:1) to afford (2R,3S,5R)-5-(6-amino-2-fluoro-9H-purin-9-yl)-2-ethynyl-2-((((((S)-1-(icosyloxy)-1-oxo-3-phenylpropan-2-yl)amino)(phenoxy)phosphoryl)oxy)methyl)tetrahydrofuran-3-yl icosanoate (800 mg, 0.628 mmol, 45.9% yield). The diastereomers were separated with Prep-SFC (Instrument: SFC-150 (Waters) Column: OZ 20×250 mm, 10 μm (Daicel), Column temperature: 35° C. Mobile phase: CO2/MeOH (0.2% ammonia/methanol)=50/50 Flow rate: 120 g/min Back pressure: 100 bar Detection wavelength: 214 nm; Cycle time: 3 min) to give first eluting isomer (Example 12A, 160 mg, 0.137 mmol, 26.7% yield) as an off-white solid; ¹H NMR (400 MHz, CDCl₃) δ 8.00 (s, 1H), 7.24-6.96 (m, 10H), 6.43 (t, J=6.8 Hz, 1H), 5.58 (dd, J=6.8, 3.7 Hz, 1H), 4.26 (ddd, J=25.6, 13.8, 6.4 Hz, 2H), 4.11 (dd, J=10.9, 5.8 Hz, 1H), 4.09-3.95 (m, 2H), 3.58 (t, J=10.8 Hz, 1H), 2.96 (d, J=6.1 Hz, 2H), 2.66-2.47 (m, 3H), 2.38 (t, J=7.6 Hz, 2H), 1.53 (s, 4H), 1.36-1.17 (m, 68H), 0.88 (t, J=6.8 Hz, 6H) and second eluting isomer (Example 12B, 150 mg, 0.127 mmol, 24.78% yield) as an off-white solid; ¹H NMR (400 MHz, CDCl₃) δ 8.00 (s, 1H), 7.24-6.95 (m, 10H), 6.41 (t, J=6.8 Hz, 1H), 5.58 (dd, J=6.8, 3.7 Hz, 1H), 4.26 (ddd, J=25.6, 13.8, 6.4 Hz, 2H), 4.11 (dd, J=10.9, 5.8 Hz, 1H), 4.09-3.95 (m, 2H), 3.58 (t, J=10.8 Hz, 1H), 2.96 (d, J=6.1 Hz, 2H), 2.66-2.47 (m, 3H), 2.38 (t, J=7.6 Hz, 2H), 1.53 (s, 4H), 1.36-1.17 (m, 68H), 0.89 (t, J=6.8 Hz, 6H).

### Example 13

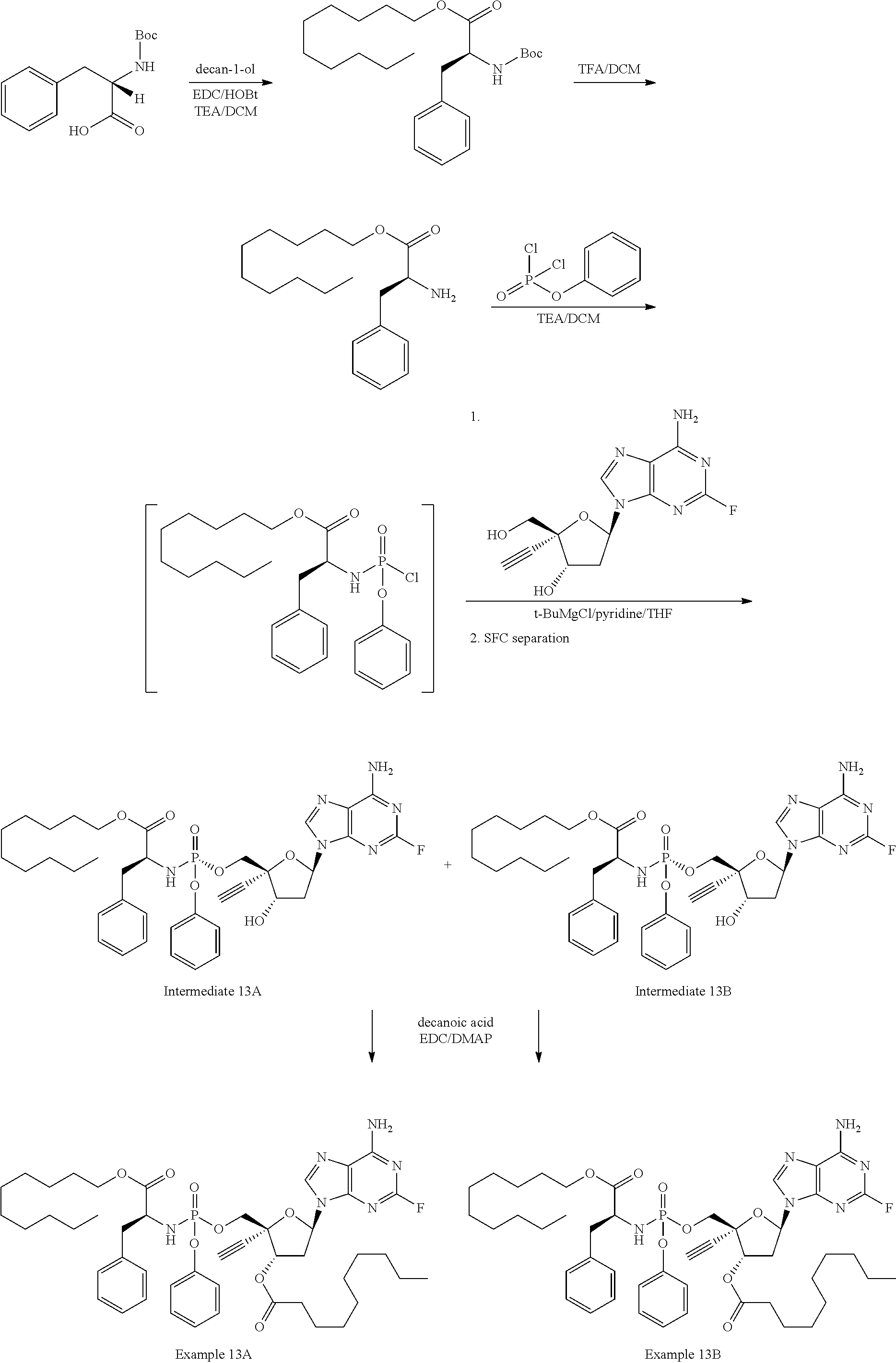

### Step 1: Decyl (tert-butoxycarbonyl)-L-phenylalaninate

To a mixture of (tert-butoxycarbonyl)-L-phenylalanine (10 g, 37.7 mmol), HOBt (7.64 g, 56.5 mmol) and EDC (8.67 g, 45.2 mmol) in DCM (150 mL) was added DIPEA (19.75 mL, 113 mmol) at 0° C. After 30 min, decan-1-ol (5.97 g, 37.7 mmol) was added. The resulting reaction mixture was stirred at 25° C. for 4 h. LCMS showed the reaction was completed. Then, the reaction was diluted with water (100 mL), the organic layer separated and the aqueous layer extracted with DCM (200 mL×2). The combined organic layers were washed with brine, dried over Na₂SO₄, filtered and concentrated. The residue was purified by silica gel chromatography (pet. ether:EtOAc=10:1) to give decyl (tert-butoxycarbonyl)-L-phenylalaninate (13 g, 32.1 mmol, 85% yield) as colorless oil. LCMS: (M+Na)=428.1; Retention time (0.1% TFA)=2.69 min.

### Step 2: Decyl L-phenylalaninate

To a solution of decyl (tert-butoxycarbonyl)-L-phenylalaninate (13 g, 32.1 mmol) in DCM (100 mL) was added TFA (19.76 mL, 256 mmol) at 0° C. The resulting reaction mixture was stirred at 25° C. for 16 h. LCMS showed the reaction was completed. The reaction mixture was concentrated, the residue was diluted with DCM (100 mL) and neutralized with NaHCO₃ (50 mL). The organic layer was separated and the aqueous layer was extracted with DCM (2×100 mL). The combined organic layers were washed with brine (80 mL), dried over Na₂SO₄, filtered and concentrated. The residue was purified by silica gel chromatography (pet. ether:EtOAc=2:1) to give decyl L-phenylalaninate (8.2 g, 26.8 mmol, 84% yield) as yellow oil. LCMS (M+H)=306.1; Retention time (0.1% TFA)=1.74 min.

### Step 3: Decyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred mixture of decyl L-phenylalaninate (4.17 g, 13.64 mmol), triethylamine (1.380 g, 13.64 mmol) in DCM (60 mL) was added dropwise phenyl phosphorodichloridate (2.88 g, 13.64 mmol) in DCM (10 mL) at about 0° C. and stirred at 0° C. for 1 h. To a stirred solution of (2R,3S,5R)-5-(6-amino-2-fluoro-9H-purin-9-yl)-2-ethynyl-2-(hydroxymethyl)tetrahydrofuran-3-ol (2 g, 6.82 mmol) in THF (40.00 mL) and pyridine (20.0 mL) was added dropwise tert-butylmagnesium chloride (1.993 g, 17.05 mmol) under an atmosphere of nitrogen at 0° C. and stirred at the same temperature for 30 min. The first prepared mixture was added dropwise to the second prepared mixture at 0° C. and stirred at 0° C. for 2 h. LCMS indicated completion of reaction. The reaction mixture was filtered and concentrated. The residue was diluted with EtOAc (600 mL) and washed with water (100 mL×2). The organic layer was dried over Na₂SO₄ and concentrated. The residue was purified by reverse phase chromatography (SepaFlash@ C18 column, BOSTON, 0-100% CH₃CN/10 mM NH₄HCO₃H2O) to give decyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (900 mg) as white solid. The diastereomers were separated with Prep-SFC (Instrument: SFC-80 (Thar, Waters), Column: AS 20×250 mm, 10 μm (Daicel), Column temperature: 40° C., Mobile phase: CO₂/MeOH (1.0% ammonia/methanol)=60/40, Flow rate: 80 g/min, Back pressure: 100 bar, Detection wavelength: 260 nm, Cycle time: 10 min, Sample solution: 900 mg dissolved in 30 mL methanol, Injection volume: 2 mL) to give first eluting isomer (Intermediate 13A, 160 mg, 0.209 mmol, 3.06% yield) as white semi-solid. LCMS (M+H)=737.2; Retention time (0.05% TFA)=1.99 min. and the second eluting isomer (Intermediate 13B, 300 mg, 0.407 mmol, 5.97% yield) as white solid. LCMS (M+H)=737.2; Retention time (0.05% TFA)=1.98 min.

### Step 4: (2R,3S,5R)-5-(6-Amino-2-fluoro-9H-purin-9-yl)-2-((((((S)-1-(decyloxy)-1-oxo-3-phenylpropan-2-yl)amino)(phenoxy)phosphoryl)oxy)methyl)-2-ethynyltetrahydrofuran-3-yl decanoate

To a stirred solution of decanoic acid (38.6 mg, 0.224 mmol) in DCM (10 mL) was added 3-(((ethylimino)methylene)amino)-N,N-dimethylpropan-1-amine hydrochloride (195 mg, 1.018 mmol) and N,N-dimethylpyridin-4-amine (124 mg, 1.018 mmol) and the resulting mixture was stirred for 0.5 h at room temperature. Then, decyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 13A, 150 mg, 0.204 mmol) was added and the resulting mixture was stirred overnight at room temperature. TLC showed the reaction was completed. The reaction mixture was partitioned between DCM (100 mL) and H₂O (30 mL). Then, the organic layer was washed with brine, dried over Na₂SO₄, filtered and concentrated. The residue was purified by reverse phase chromatography (SepaFlash@ C18 column, BOSTON, 0-100% CH₃OH/10 mM NH₄HCO₃H2O) to give (2R,3S,5R)-5-(6-amino-2-fluoro-9H-purin-9-yl)-2-((((((S)-1-(decyloxy)-1-oxo-3-phenylpropan-2-yl)amino)(phenoxy)phosphoryl)oxy)methyl)-2-ethynyltetrahydrofuran-3-yl decanoate (114 mg, 0.125 mmol, 61.6% yield) as colorless oil. LCMS (M+H)=891; Retention time (0.05% TFA)=3.29 min. HPLC Retention time (10 mM NH₄HCO₃)=11.90 min. ¹H NMR (400 MHz, CDCl₃) δ 8.01 (s, 1H), 7.27-7.22 (m, 4H), 7.21-7.15 (m, 3H), 7.14-7.11 (m, 3H), 6.42 (t, J=6.8 Hz, 1H), 6.00 (brs, 2H), 5.59 (dd, J=6.8, 4.0 Hz, 1H), 4.38-4.31 (m, 1H), 4.27 (dd, J=10.8, 6.4 Hz, 1H), 4.15 (dd, J=11.2, 6.0 Hz, 1H), 4.04 (t, J=6.8 Hz, 2H), 3.66 (t, J=6.8 Hz, 1H), 3.02 (d, J=6.4 Hz, 2H), 2.70-2.57 (m, 3H), 2.42 (t, J=7.6 Hz, 2H), 1.69 (quint, J=7.2 Hz, 2H), 1.54 (quint, J=7.2 Hz, 2H), 1.35-1.23 (m, 26H), 0.93-0.88 (m, 6H).

To a stirred solution of decanoic acid (51.4 mg, 0.299 mmol) in DCM (10 mL) were added 3-(((ethylimino)methylene)amino)-N,N-dimethylpropan-1-amine hydrochloride (260 mg, 1.357 mmol) and N,N-dimethylpyridin-4-amine (166 mg, 1.357 mmol) and the resulting mixture was stirred for 0.5 h at room temperature. Then, decyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (Intermediate 13A, 200 mg, 0.271 mmol) was added and the resulting mixture was stirred overnight at room temperature. TLC showed the reaction was completed. The reaction mixture was partitioned between DCM (100 mL) and H₂O (30 mL). Then, the organic layer was washed with brine, dried over Na₂SO₄, filtered and concentrated. The residue was purified by reverse phase chromatography (SepaFlash@ C18, BOSTON, 0-100% CH₃OH/10 mM NH₄HCO₃H2O) to give (2R,3S,5R)-5-(6-amino-2-fluoro-9H-purin-9-yl)-2-((((((S)-1-(decyloxy)-1-oxo-3-phenylpropan-2-yl)amino)(phenoxy)phosphoryl)oxy)methyl)-2-ethynyltetrahydrofuran-3-yl decanoate (100 mg, 0.110 mmol, 40.6% yield) as white solid. LCMS (M+H)=891; Retention time (0.05% TFA)=3.26 min. HPLC Retention time (10 mM NH₄HCO₃)=11.81 min. ¹H NMR (400 MHz, CDCl₃) δ 8.01 (s, 1H), 7.25-7.10 (m, 8H), 7.06-7.02 (m, 2H), 6.45-6.41 (m, 1H), 6.20 (brs, 2H), 5.58 (dd, J=6.8, 3.6 Hz, 1H), 4.29 (dd, J=11.2, 6.8 Hz, 1H), 4.26-4.19 (m, 1H), 4.10 (dd, J=11.2, 6.0 Hz, 1H), 4.01 (dt, J=6.8, 2.0 Hz, 2H), 3.73 (t, J=10.8 Hz, 1H), 2.96 (d, J=6.4 Hz, 2H), 2.61 (s, 1H), 2.58 (t, J=7.2 Hz, 1H), 2.51 (ddd, J=14.0, 6.4, 4.0 Hz, 1H), 2.38 (t, J=7.6 Hz, 2H), 1.65 (quint, J=7.2 Hz, 2H), 1.53 (quint, J=7.2 Hz, 2H), 1.30-1.23 (m, 26H), 0.91-0.87 (m, 6H).

### Example 14

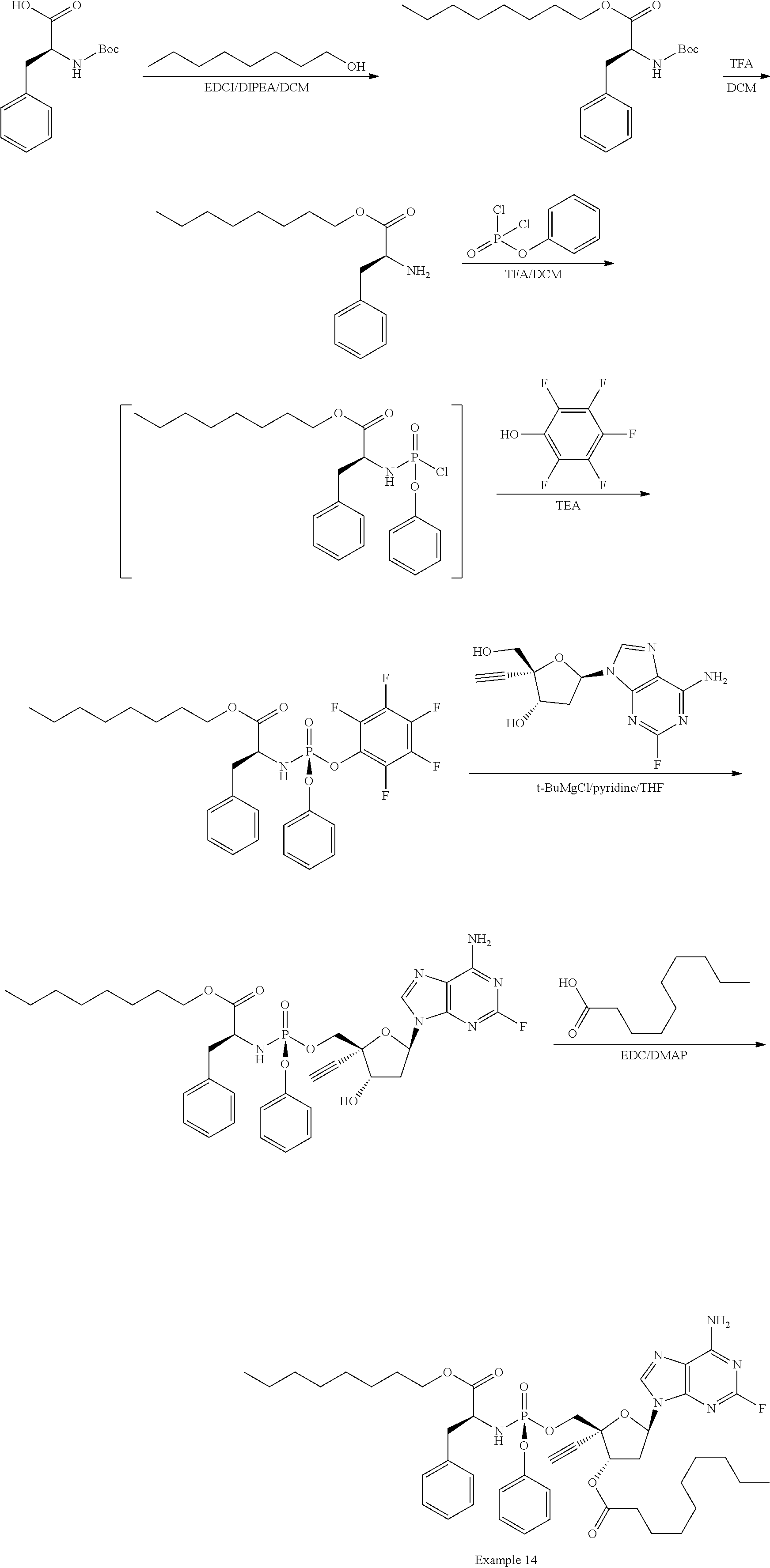

### Step-1: octyl (tert-butoxycarbonyl)-L-phenylalaninate

To a stirred mixture of EDC (17.34 g, 90 mmol), HOBt (11.54 g, 75 mmol) and (tert-butoxycarbonyl)-L-phenylalanine (20.0 g, 75 mmol) in DCM (100 mL) was added DIPEA (39.5 mL, 226 mmol) at −10° C. After 30 min, a solution of octan-1-ol (11.78 g, 90 mmol) in DCM (100 mL) was added to the above solution and stirred at 25° C. for 16 h. TLC showed the reaction was completed. The reaction mixture was diluted with water (100 mL), the organic layer was separated and the aqueous layer was extracted with DCM (80 mL×3). The combined organic phases were washed with brine (80 mL), dried over Na₂SO₄ and concentrated. The residue was purified by silica gel chromatography (pet. ether:EtOAc=10:1) to afford octyl (tert-butoxycarbonyl)-L-phenylalaninate (18.0 g, 45.3 mmol, 60.0% yield) as white solid. LCMS (M+H)=377.7; Retention time (0.1% TFA)=2.423 min.

### Step-2: Octyl L-phenylalaninate

A mixture of TFA (80 mL, 1038 mmol) and octyl (tert-butoxycarbonyl)-L-phenylalaninate (18.0 g, 47.7 mmol) in DCM (200 mL) was stirred at 25° C. overnight. TLC showed the reaction was completed. The reaction mixture was concentrated, and the pH of the residue was adjusted to 8-9 with NaHCO₃ (1N) and extracted with DCM (500 mL×3). The combined organic phases were washed with brine, dried over Na₂SO₄ and concentrated. The residue was purified by silica gel chromatography (DCM:MeOH=10:1) to afford octyl L-phenylalaninate (12 g, 43.3 mmol, 91% yield) as white solid. ¹H NMR (400 MHz, CDCl₃) δ 7.33-7.27 (m, 2H), 7.26-7.16 (m, 3H), 4.09 (t, J=6.7 Hz, 2H), 3.72 (dd, J=7.8, 5.4 Hz, 1H), 3.08 (dd, J=13.5, 5.4 Hz, 1H), 2.87 (dd, J=13.5, 7.8 Hz, 1H), 1.64-1.57 (m, 2H), 1.28 (s, 10H), 0.89 (t, J=6.9 Hz, 3H).

### Step-3: Octyl ((perfluorophenoxy)(phenoxy)phosphoryl)-L-phenylalaninate

To a stirred solution of octyl L-phenylalaninate (5.00 g, 18.02 mmol) in anhydrous DCM (40 mL) was added TEA (5.02 mL, 36.0 mmol) dropwise at −70° C. over 15 min. To this mixture was added a solution of phenyl phosphorodichloride (3.80 g, 18.02 mmol) in anhydrous DCM (50 mL) over 1 h. The reaction mixture was stirred at this temperature for additional 30 min and then, allowed to warm to 0° C. over 2 h and stirred for 1 h. To this mixture was added a solution of 2,3,4,5,6-pentafluorophenol (3.32 g, 18.02 mmol) and triethylamine (2.51 mL, 18.02 mmol) in DCM (30 mL) over 20 min and stirred at 0° C. for 16 h, LCMS showed the reaction was completed. The white solid (triethylamine hydrochloride) was filtered off and washed with DCM (50 mL). The filtrate was concentrated under reduced pressure and the residue was triturated with tert-butylmethyl ether (150 mL), and the triethylamine hydrochloride salt was removed by filtration. The cake was washed with tert-butylmethyl ether (2×50 mL), and the combined filtrate was concentrated under reduced pressure to give crude product (22 g) as solid containing an even mixture of diastereomers. The crude product was triturated with 10% EtOAc/hexanes (100 mL) and solids collected by filtration to give octyl ((perfluorophenoxy)(phenoxy)phosphoryl)-L-phenylalaninate (2,300 mg, 3.84 mmol, 21.28% yield) as white solid (>98% de as determined by ³¹PNMR). ¹HNMR (400 MHz, CDCl₃) δ 7.35 (t, J=7.9 Hz, 2H), 7.22 (dt, J=13.3, 5.7 Hz, 6H), 7.08-6.99 (m, 2H), 4.44 (ddd, J=15.7, 10.0, 5.8 Hz, 1H), 4.16-3.95 (m, 2H), 3.77 (t, J=11.1 Hz, 1H), 3.15 (dd, J=13.7, 5.3 Hz, 1H), 3.05 (dd, J=13.7, 6.3 Hz, 1H), 1.60-1.49 (m, 2H), 1.37-1.12 (m, 10H), 0.88 (t, J=6.9 Hz, 3H). ³¹PNMR (162 MHz, CDCl₃) δ−1.58.

### Step 4: Octyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.18 g, 0.614 mmol) in THF (36 mL) and pyridine (9.00 mL) was added dropwise tert-butylmagnesium chloride (1.289 mL, 1.289 mmol) at −15° C. After 30 min, a solution of octyl ((S)-(perfluorophenoxy)(phenoxy)phosphoryl)-L-phenylalaninate (0.552 g, 0.921 mmol) in THF (5 mL) was added dropwise to the above solution at −15° C. and stirred at −15° C. for 16 h. LCMS showed the reaction was completed. The reaction mixture was quenched with sat. NH₄Cl and extracted with EtOAc (20 mL×3). The combined organic phases were washed with brine (20 mL), dried with Na₂SO₄, filtered and concentrated in vacuum. The crude product was purified by reverse phase chromatography (SepaFlash@ C18 column, BOSTON, 40 g, 0-100% CH₃CN/10 mM NH₄HCO₃H2O) to give octyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (170 mg, 0.233 mmol, 38.0% yield) as white solid. LCMS (M+H)=709.0; Retention time (10 mM NH₄HCO₃)=1.974 min.

### Step 5: (2R,3S,5R)-5-(6-Amino-2-fluoro-9H-purin-9-yl)-2-ethynyl-2-((((S)-(((S)-1-(octyloxy)-1-oxo-3-phenylpropan-2-yl)amino)(phenoxy)phosphoryl)oxy)methyl)tetrahydrofuran-3-yl decanoate

To a stirred solution of decanoic acid (45.5 mg, 0.264 mmol) in DCM (4 mL) was added DMAP (147 mg, 1.199 mmol) and EDC (230 mg, 1.199 mmol), and the resulting mixture was stirred for 0.5 h at room temperature. Then, octyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (170 mg, 0.240 mmol) in DCM (2 mL) was added, and the resulting mixture was stirred overnight at room temperature. The LCMS showed the reaction was completed. The mixture was diluted with water (10 mL) and extracted with DCM (20 mL×3). The organic phases were combined, washed with brine (20 mL), dried over Na₂SO₄ and concentrated under vacuum. The residue was purified by reverse phase chromatography (SepaFlash@ C18 column, 0-100% CH₃CN/10 mM NH₄HCO₃H2O) to give (2R,3S,5R)-5-(6-amino-2-fluoro-9H-purin-9-yl)-2-ethynyl-2-((((S)-(((S)-1-(octyloxy)-1-oxo-3-phenylpropan-2-yl)amino)(phenoxy)phosphoryl)oxy)methyl)tetrahydrofu-ran-3-yl decanoate (129 mg, 0.140 mmol, 58.4% yield) as light yellow solid. LCMS: Retention time (0.1% TFA)=2.746 min; HPLC: Retention time (10 mM NH₄HCO₃)=9.376 min. ¹H NMR (400 MHz, CDCl₃) δ 8.03 (s, 1H), 7.25-7.18 (m, 5H), 7.15 (d, J=8.5 Hz, 2H), 7.14-7.09 (m, 3H), 6.39 (t, J=6.7 Hz, 1H), 5.99 (s, 2H), 5.56 (dd, J=6.3, 4.5 Hz, 1H), 4.32 (ddd, J=16.6, 10.3, 6.4 Hz, 1H), 4.25 (dd, J=11.1, 6.3 Hz, 1H), 4.13 (dd, J=11.1, 5.7 Hz, 1H), 4.02 (t, J=6.8 Hz, 2H), 3.63 (t, J=10.7 Hz, 1H), 3.00 (d, J=6.6 Hz, 2H), 2.62 (s, 1H), 2.62-2.57 (m, 2H), 2.40 (t, J=7.6 Hz, 2H), 1.70-1.62 (m, 2H), 1.55-1.48 (m, 2H), 1.32-1.26 (m, 12H), 1.24 (s, 10H), 0.88 (t, J=6.7 Hz, 6H).

### Example 15

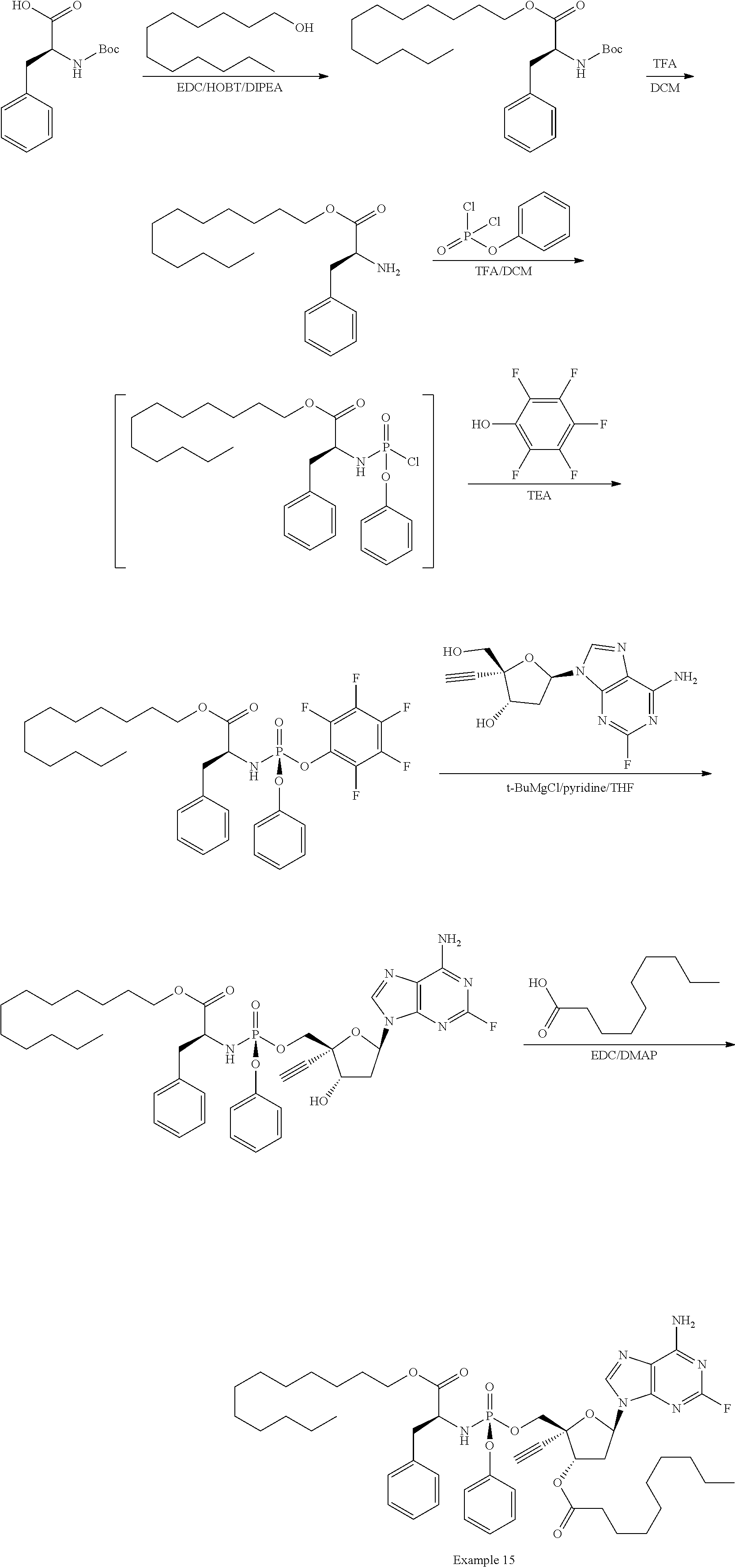

### Step 1: Dodecyl (tert-butoxycarbonyl)-L-phenylalaninate

To a stirred mixture of (tert-butoxycarbonyl)-L-phenylalanine (15 g, 56.5 mmol), HOBt (11.46 g, 85 mmol) and EDC (13.01 g, 67.8 mmol) in DCM (150 mL) was added TEA (7.88 mL, 56.5 mmol) at 0° C. After stirring for 30 min, dodecan-1-ol (10.54 g, 56.5 mmol) was added. The resulting reaction mixture was stirred at 25° C. for 16 h. LCMS showed the reaction was completed. The reaction mixture was diluted with water (80 mL) and extracted with DCM (100 mL×2). The combined organic layers were washed with brine, dried over Na₂SO₄ and concentrated. The residue was purified by silica gel chromatography (pet. ether:EtOAc=10:1) to give dodecyl (tert-butoxycarbonyl)-L-phenylalaninate (14.2 g, 32.2 mmol, 57.0% yield) as a yellow oil. LCMS (M+Na)=456.4; Retention time (0.05% TFA)=3.20 min.

### Step 2: Dodecyl L-phenylalaninate

To a stirred solution of dodecyl (tert-butoxycarbonyl)-L-phenylalaninate (14.6 g, 33.7 mmol) in DCM (150 mL) was added TFA (31.1 mL, 404 mmol) at 0° C. The resulting reaction mixture was stirred at 25° C. for 4 h. LCMS showed the reaction was completed. The reaction mixture was concentrated and the residue was diluted with DCM (100 mL) and neutralized with NaHCO₃ (50 mL). The organic layer was separated and the aqueous layer was extracted with DCM (2×100 mL). The combined organic layers were washed with brine (80 mL), dried over Na₂SO₄, filtered and concentrated. The residue was purified by silica gel chromatography (pet. ether:EtOAc=2:1) to give dodecyl L-phenylalaninate (9.4 g, 27.6 mmol, 82% yield) as colorless oil. LCMS (M+H)=334.3; Retention time (0.05% TFA)=1.88 min.

### Step 3: Dodecyl ((S)-(perfluorophenoxy)(phenoxy)phosphoryl)-L-phenylalaninate

To a stirred solution of dodecyl L-phenylalaninate (4 g, 11.99 mmol) in anhydrous DCM (25 mL) was added dropwise triethylamine (1.834 mL, 13.19 mmol) at −70° C. To this mixture was added dropwise a solution of phenyl phosphorodichloridate (2.505 g, 11.87 mmol) in anhydrous DCM (8 mL). The reaction mixture was stirred at this temperature for additional 30 min and then allowed to warm to 0° C. over 2 h and stirred for 1 h. To this mixture was added dropwise a solution of 2,3,4,5,6-pentafluorophenol (2.185 g, 11.87 mmol) and triethylamine (1.834 mL, 13.19 mmol) in DCM (30 mL) and stirred at 0° C. for 2 h. LCMS showed the reaction was completed. The white solid (triethylamine hydrochloride) was filtered off and washed with DCM (10 mL). The filtrate was concentrated under reduced pressure, the residue was triturated with tert-butylmethyl ether (20 mL), and the triethylamine hydrochloride salt was removed by filtration. The cake was washed with tert-butylmethyl ether (2×10 mL), and the filtrate was concentrated under reduced pressure. The mixture was triturated with 10% EtOAc/hexanes (30 mL) and the solids were collected by filtration to give dodecyl ((S)-(perfluorophenoxy)(phenoxy)phosphoryl)-L-phenylalaninate (1.8 g, 2.57 mmol, 21.39% yield) as white solid ((>98% de as determined by ³¹P NMR). LCMS (M+H)=656.2; Retention time (0.05% TFA)=3.31 min. ³¹P NMR (162 MHz, DMSO-d₆) δ 0.21.

### Step 3: Dodecyl ((R)-(perfluorophenoxy)(phenoxy)phosphoryl)-L-phenylalaninate

To a stirred solution of dodecyl L-phenylalaninate (5.9 g, 17.69 mmol) in anhydrous DCM (40 mL) was added TEA (2.71 mL, 19.46 mmol) dropwise at −70° C. over 15 min. To this mixture was added a solution of phenyl phosphorodichloride (3.73 g, 17.69 mmol) in anhydrous DCM (50 mL) over 1 h. The reaction mixture was stirred at this temperature for additional 30 min and then allowed to warm to 0° C. over 2 h and stirred for 1 h. To this mixture was added a solution of 2,3,4,5,6-pentafluorophenol (3.26 g, 17.69 mmol) and triethylamine (2.71 mL, 19.46 mmol) in DCM (30 mL) over 20 min and stirred at 0° C. for 16 h. TLC showed the reaction was completed. The white solid (triethylamine hydrochloride) was filtered off and washed with DCM (50 mL). The filtrate was concentrated under reduced pressure, the residue was triturated with tert-butylmethyl ether (150 mL) and the triethylamine hydrochloride salt was removed by filtration. The cake was washed with tert-butylmethyl ether (2×50 mL), and the combined filtrate was concentrated under reduced pressure to give solid containing an even mixture of diastereomers. The crude product was triturated with 20% EtOAc/hexanes (100 mL) and solids collected by filtration to afford dodecyl ((R)-(perfluorophenoxy)(phenoxy)phosphoryl)-L-phenylalaninate (2.3 g, 3.51 mmol, 19.83% yield, >98% de as determined by ³¹PNMR)). ¹HNMR (400 MHz, CDCl₃) δ 7.40-7.30 (m, 2H), 7.25-7.15 (m, 6H), 7.09-7.03 (m, 2H), 4.52-4.36 (m, 1H), 4.16-3.97 (m, 2H), 3.88 (dd, J=19.3, 10.1 Hz, 1H), 3.14 (dd, J=13.7, 5.4 Hz, 1H), 3.10-2.99 (m, 1H), 1.61-1.46 (m, 2H), 1.38-1.16 (m, 18H), 0.88 (t, J=6.7 Hz, 3H). ³¹PNMR (162 MHz, CDCl₃) δ−1.56.

### Step 4: Dodecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (300 mg, 1.023 mmol) in anhydrous THF (60 mL) and pyridine (15.0 mL) was added dropwise tert-butylmagnesium chloride (2.046 mL, 2.046 mmol) at −15° C. To this mixture was added dropwise a solution of dodecyl ((S)-(perfluorophenoxy)(phenoxy)phosphoryl)-L-phenylalaninate (939 mg, 1.432 mmol) in anhydrous THF (20 mL) over 1 h. The reaction mixture was stirred at this temperature overnight. LCMS showed the reaction was completed. The reaction mixture was quenched with 2N NH₄Cl (10 mL) and water (50 mL), and extracted with EtOAc (2×150 mL). Then, the combined organic layers were washed with brine (50 mL), dried over Na₂SO₄, filtered and concentrated. The residue was purified by reverse phase column chromatography (SepaFlash@ C18 column, BOSTON, 80 g 0-100%, CH₃OH/10 mM NH₄HCO₃H2O) to give dodecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (190 mg, 0.248 mmol, 24.28% yield) as white solid. LCMS (M+H)=764.9; Retention time (10 mM NH₄HCO₃)=2.44 min.

### Step 4: Dodecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.36 g, 1.228 mmol) in THF (100 mL) and pyridine (2.00 mL) was added dropwise tert-butylmagnesium chloride (2.58 mL, 2.58 mmol) at 0° C. and the reaction was stirred at 25° C. for 30 min. A solution of dodecyl ((S)-(perfluorophenoxy)(phenoxy)phosphor-yl)-L-phenylalaninate (0.805 g, 1.228 mmol) in THF (5 mL) was added dropwise to the above solution at −15° C. and stirred at −15° C. for 4 h. TLC showed the presence of new compound. The reaction was quenched with 2M HCl (10 mL) and extracted with EtOAc (50 mL×3). The combined organic phases were washed with a solution of NaHCO₃ (50 mL), brine (20 mL), dried with Na₂SO₄, filtered and concentrated in vacuum. The crude product was purified by silica gel chromatography (DCM:MeOH=20:1) to afford dodecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (270 mg, 0.353 mmol, 28.8% yield) as white solid. LCMS (M+H)+=765.7; Retention time (0.1% TFA)=2.41 min.

### Step 5: (2R,3S,5R)-5-(6-Amino-2-fluoro-9H-purin-9-yl)-2-((((S)-(((S)-1-(dodecyloxy)-1-oxo-3-phenylpropan-2-yl)amino)(phenoxy)phosphoryl)oxy)methyl)-2-ethynyltetrahydrofuran-3-yl decanoate

To a cold (ice-water bath) stirred solution of decanoic acid (37.2 mg, 0.216 mmol) in DCM (15 mL) was added 3-(((ethylimino)methylene)amino)-N,N-dimethylpropan-1-amine hydrochloride (188 mg, 0.981 mmol) and N,N-dimethylpyridin-4-amine (120 mg, 0.981 mmol). After 0.5 h, dodecyl ((S)-(((2R,3S,5R)-5-(6-amino-2-fluoro-9H-purin-9-yl)-2-ethynyl-3-hydroxytetrahydro-furan-2-yl)methoxy)(phenoxy)phosphor-yl)-L-phenylalaninate (150 mg, 0.196 mmol) was added and the resulting mixture was stirred at 25° C. for 16 h. TLC showed the reaction was completed. The reaction mixture was partitioned between DCM (100 mL) and H₂O (30 mL). Then, the organic layer was washed with brine (15 mL), dried over Na₂SO₄, filtered and concentrated. The residue was purified by reverse phase chromatography (SepaFlash@ C18 column, BOSTON, 40 g, 0-100% CH₃OH/10 mM NH₄HCO₃H2O) to give (2R,3S,5R)-5-(6-amino-2-fluoro-9H-purin-9-yl)-2-((((S)-(((S)-1-(dodecyloxy)-1-oxo-3-phenylpropan-2-yl)amino)(phenoxy)phosphoryl)oxy)methyl)-2-ethynyltetrahydrofuran-3-yl decanoate (129 mg, 0.138 mmol, 70.5% yield) as colorless oil. LCMS (M+H)=919.3; Retention time (0.1% TFA)=3.52 min; purity: 100% (254 nm). HPLC Retention time (10 mM NH₄HCO₃)=11.12 min. ¹H NMR (400 MHz, CDCl₃) δ 7.99 (s, 1H), 7.25-7.09 (m, 10H), 6.40 (t, J=6.8 Hz, 1H), 5.99 (brs, 2H), 5.56 (dd, J=6.4, 4.4 Hz, 1H), 4.36-4.29 (m, 1H), 4.25 (dd, J=11.2, 6.4 Hz, 1H), 4.13 (dd, J=10.8, 5.6 Hz, 1H), 4.02 (t, J=6.8 Hz, 2H), 3.64 (t, J=10.8 Hz, 1H), 3.00 (d, J=6.4 Hz, 2H), 2.67-2.56 (m, 3H), 2.40 (t, J=7.6 Hz, 2H), 1.67 (quint, J=7.2 Hz, 2H), 1.52 (quint, J=7.2 Hz, 2H), 1.30-1.20 (m, 30H), 0.91-0.86 (m, 6H).

### Example 16

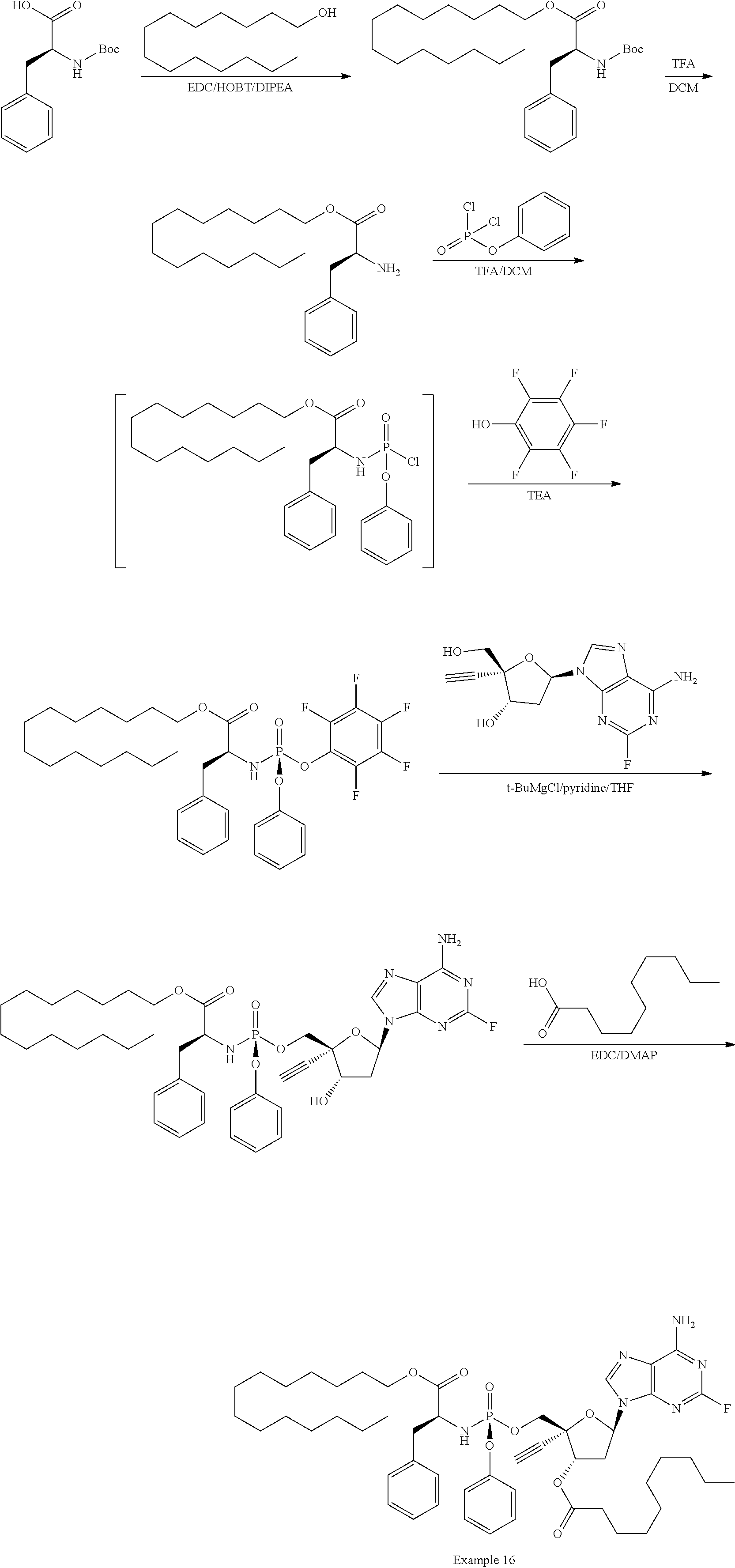

### Step 1: Tetradecyl (tert-butoxycarbonyl)-L-phenylalaninate

To a stirred mixture of (tert-butoxycarbonyl)-L-phenylalanine (15 g, 56.5 mmol), HOBt (4.33 g, 28.3 mmol), EDC (16.26 g, 85 mmol) in DCM (120 mL) was added DIPEA (29.6 mL, 170 mmol) at 0° C. After 30 min, tetradecan-1-ol (12.12 g, 56.5 mmol) in DCM (20 mL) was added and stirred at 25° C. for 16 h. LCMS showed the presence of new compound. The reaction mixture was diluted with water (80 mL), the organic layer was separated and the aqueous phase was extracted with DCM (80 mL×2). The combined organic phases were washed with brine, dried over Na₂SO₄ and concentrated. The residue was purified by silica gel chromatography (Santai, 120 g, pet. ether:EtOAc=30:1) to afford tetradecyl (tert-butoxycarbonyl)-L-phenylalaninate (20 g, 39.4 mmol, 69.8% yield) as yellow oil. LCMS (M+Na)=484.2, Retention time (10% TFA)=3.532 min.

### Step 2: Tetradecyl L-phenylalaninate

To a stirred mixture of tetradecyl (tert-butoxycarbonyl)-L-phenylalaninate (20 g, 43.3 mmol) in DCM (160 mL) was added TFA (28.6 mL, 372 mmol) at 0° C., and stirred at 25° C. for 4 h. LCMS showed the presence of new compound. The reaction was concentrated and the pH of the residue was adjusted to 8-9 with NaOH (1 N) and the mixture was extracted with DCM (100 mL×3). The combined organic phases were washed with brine, dried over Na₂SO₄ and concentrated. The residue was purified by silica gel chromatography (Santai, 80 g, DCM:MeOH=10:1) to afford tetradecyl L-phenylalaninate (12 g, 33.2 mmol, 77% yield) as yellow oil. LCMS (M+H)=362.4, Retention time (0.1% TFA)=2.506 min.

### Step 3: Tetradecyl ((perfluorophenoxy)(phenoxy)phosphoryl)-L-phenylalaninate

To a stirred solution of tetradecyl L-phenylalaninate (6.0 g, 16.59 mmol) in anhydrous DCM (100 mL) was added dropwise triethylamine (2.54 mL, 18.25 mmol) at −70° C. over 15 min. To this mixture was added a solution of phenyl phosphorodichloridate (3.47 g, 16.43 mmol) in anhydrous DCM (35 mL) over 1 h. The reaction mixture was stirred at this temperature for additional 30 min and then allowed to warm to 0° C. over 2 h and stirred for 1 h. To this mixture was added a solution of 2,3,4,5,6-pentafluorophenol (3.02 g, 16.43 mmol) and triethylamine (2.54 mL, 18.25 mmol) in DCM (30 mL) over 20 min and stirred at 0° C. for 4 h. LCMS showed the reaction was completed. The white solid (triethylamine hydrochloride) was filtered off and washed with DCM (50 mL). The filtrate was concentrated under reduced pressure, and the residue was triturated with tert-butylmethyl ether (250 mL), then, triethylamine hydrochloride salt was removed by filtration. The cake was washed with tert-butylmethyl ether (2×50 mL), and the combined filtrate was concentrated under reduced pressure to give 26 g of crude solid containing an even mixture of diastereomers. The crude product was triturated with 20% EtOAc/hexanes (200 mL) and solids collected by filtration to afford tetradecyl ((S)-(perfluorophenoxy)(phenoxy)phosphoryl)-L-phenylalaninate (5.0 g, 7.31 mmol, 44.1% yield) as white solid (>98% de as determined by ³¹PNMR: N76107-92-A1). ¹HNMR (400 MHz, CDCl₃) δ 7.35 (t, J=7.9 Hz, 2H), 7.26-7.15 (m, 6H), 7.12-7.00 (m, 2H), 4.50-4.38 (m, 1H), 4.15-3.95 (m, 2H), 3.85-3.70 (m, 1H), 3.10 (ddd, J=37.7, 13.4, 5.5 Hz, 2H), 1.54 (s, 2H), 1.36-1.14 (m, 22H), 0.88 (t, J=6.8 Hz, 3H). ³¹PNMR (162 MHz, CDCl₃) δ−1.57.

### Step 4: Tetradecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.6 g, 2.046 mmol) in THF (120 mL) and pyridine (30.0 mL) was added dropwise tert-butylmagnesium chloride (4.30 mL, 4.30 mmol) at −15° C. Then, the reaction was stirred at 25° C. for 30 min. A solution of tetradecyl ((S)-(perfluorophenoxy)(phenoxy))-L-phosphorylphenylalaninate (2.098 g, 3.07 mmol) in THF (12.5 mL) was added dropwise to the above solution at −15° C. and stirred at −15° C. for 4 h. LCMS showed the reaction was completed. The reaction was quenched with sat. NH₄Cl (20 mL) and concentrated to remove THF. The resulting aqueous mixture was extracted with EtOAc (20 mL×3) and the combined organic phases were washed with a solution of NH₄Cl (20 mL), brine (20 mL), dried with Na₂SO₄, filtered and concentrated in vacuum. The crude product was purified by reverse phase chromatography (SepaFlash@ C18 column, 80 g, 0-100% CH₃CN/10 mM NH₄HCO₃H2O) to afford tetradecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (760 mg, 0.857 mmol, 41.9% yield) as white solid. LCMS (M+H)=793.0; Retention time (10 mM NH₄HCO₃)=2.767 min.

### Step 5: (2R,3S,5R)-5-(6-Amino-2-fluoro-9H-purin-9-yl)-2-ethynyl-2-((((S)-(((S)-1-oxo-3-phenyl-1-(tetradecyloxy)propan-2-yl)amino)(phenoxy)phosphoryl)oxy)methyl)tetrahydrofuran-3-yl decanoate

To a stirred solution of decanoic acid (47.8 mg, 0.277 mmol) in DCM (4 mL) was added DMAP (154 mg, 1.261 mmol) and EDC (242 mg, 1.261 mmol) and stirred for 0.5 h at room temperature. Then, tetradecyl ((S)-(((2R,3S,5R)-5-(6-amino-2-fluoro-9H-purin-9-yl)-2-ethynyl-3-hydroxytetra-hydrofuran-2-yl)methoxy)(phenoxy)phosphoryl)-L-phenylalaninate (200 mg, 0.252 mmol) was added and stirred for 16 h at room temperature. TLC (DCM:MeOH=17:1, Rf=0.45) showed presence of new compound. The mixture was quenched with water (10 mL) and extracted with DCM (20 mL×3). The combined organic phases were washed with brine, dried over Na₂SO₄ and concentrated under vacuum. The residue was purified by prep-TLC (DCM:MeOH=17:1, Rf=0.45) to give (2R,3S,5R)-5-(6-amino-2-fluoro-9H-purin-9-yl)-2-ethynyl-2-((((S)-(((S)-1-oxo-3-phenyl-1-(tetradecyloxy)pro-pan-2-yl)amino)(phenoxy)phosphoryl)oxy)methyl)tetrahydrofuran-3-yl decanoate (137 mg, 0.139 mmol, 55.2% yield) as light yellow solid. LCMS (M+H)=947.7; Retention time (0.1% TFA)=4.127 min. HPLC: Retention time (10 mM NH₄HCO₃)=14.764 min. ¹H NMR (400 MHz, CDCl₃) δ 8.01 (s, 1H), 7.25-7.21 (d, J=7.6 Hz, 4H), 7.20 (dd, J=6.4, 2.1 Hz, 1H), 7.15 (d, J=8.6 Hz, 2H), 7.13-7.08 (m, 3H), 6.39 (t, J=6.7 Hz, 1H), 5.99 (s, 2H), 5.57 (dd, J=6.6, 4.3 Hz, 1H), 4.32 (ddd, J=9.6, 8.1, 4.9 Hz, 1H), 4.25 (dd, J=11.1, 6.3 Hz, 1H), 4.13 (dd, J=11.1, 5.7 Hz, 1H), 4.02 (t, J=6.8 Hz, 2H), 3.63 (t, J=10.7 Hz, 1H), 3.00 (d, J=6.4 Hz, 2H), 2.66-2.54 (m, 3H), 2.40 (t, J=7.6 Hz, 2H), 1.71-1.62 (m, 2H), 1.55-1.48 (m, 2H), 1.27 (d, J=2.5 Hz, 9H), 1.25 (d, J=4.9 Hz, 24H), 0.90-0.86 (m, 6H).

### Example 17

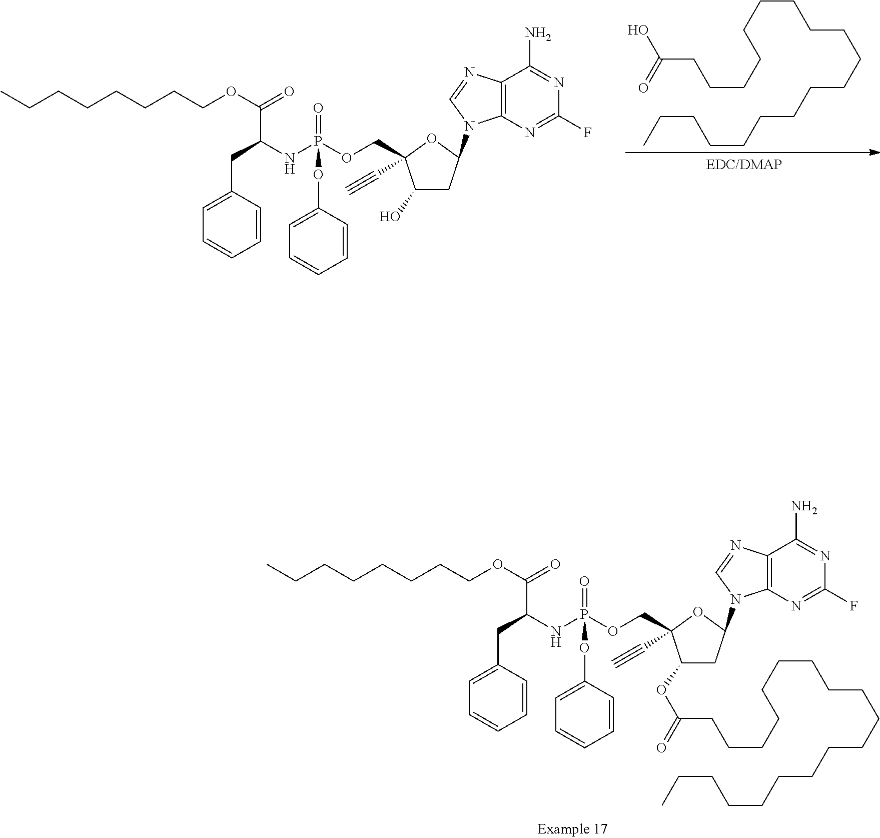 [SMILES: C#C[C@]1(CO[P@@](=O)(N[C@@H](Cc2ccccc2)C(=O)OCCCCCCCC)Oc2ccccc2)O[C@@H](n2cnc3c(N)nc(F)nc32)C[C@@H]1O ; CCCCCCCCCCCCCCCCCCCC(=O)O ; C#C[C@]1(CO[P@@](=O)(N[C@@H](Cc2ccccc2)C(=O)OCCCCCCCC)Oc2ccccc2)O[C@@H](n2cnc3c(N)nc(F)nc32)C[C@@H]1OC(=O)CCCCCCCCCCCCCCCCCCC]

### (2R,3S,5R)-5-(6-Amino-2-fluoro-9H-purin-9-yl)-2-ethynyl-2-((((S)-(((S)-1-(octyloxy)-1-oxo-3-phenylpropan-2-yl)amino)(phenoxy)phosphoryl)oxy)methyl)tetrahydrofuran-3-yl icosanoate

To a solution of icosanoic acid (97 mg, 0.310 mmol) in DCM (4 mL) was added DMAP (172 mg, 1.411 mmol) and EDC (270 mg, 1.411 mmol) and stirred for 0.5 h at room temperature. Then, octyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (N77883-37, 200 mg, 0.282 mmol) in DCM (3 mL) was added and stirred for 16 h at room temperature. TLC (DCM:MeOH=17:1, Rf=0.45) showed presence of new compound. The reaction mixture was quenched with water (10 mL) and extracted with DCM (20 mL×3). The combined organic phases were washed with brine, dried over Na₂SO₄ and concentrated under vacuum. The residue was purified by prep-TLC (DCM:CH₃OH=17:1, Rf=0.45) to give (2R,3S,5R)-5-(6-amino-2-fluoro-9H-purin-9-yl)-2-ethynyl-2-((((S)-(((S)-1-(octyloxy)-1-oxo-3-phenylpropan-2-yl)amino)(phenoxy)phosphoryl)oxy)methyl)tetrahydrofuran-3-yl icosanoate (170 mg, 0.162 mmol, 57.4% yield) as white solid. HPLC: Retention time (10 mM NH₄HCO₃): 15.683 min. ¹H NMR (400 MHz, CDCl₃) δ 8.00 (s, 1H), 7.22 (d, J=7.9 Hz, 4H), 7.20-7.18 (m, 1H), 7.15 (d, J=8.6 Hz, 2H), 7.13-7.08 (m, 3H), 6.39 (t, J=6.7 Hz, 1H), 6.08 (s, 2H), 5.57 (dd, J=6.6, 4.2 Hz, 1H), 4.36-4.28 (m, 1H), 4.25 (dd, J=11.1, 6.3 Hz, 1H), 4.13 (dd, J=11.1, 5.7 Hz, 1H), 4.02 (t, J=6.8 Hz, 2H), 3.68 (t, J=10.7 Hz, 1H), 3.00 (d, J=6.4 Hz, 2H), 2.67-2.53 (m, 3H), 2.39 (t, J=7.6 Hz, 2H), 1.71-1.61 (m, 2H), 1.56-1.47 (m, 2H), 1.25 (d, J=4.2 Hz, 42H), 0.88 (t, J=6.7 Hz, 6H).

### Example 18

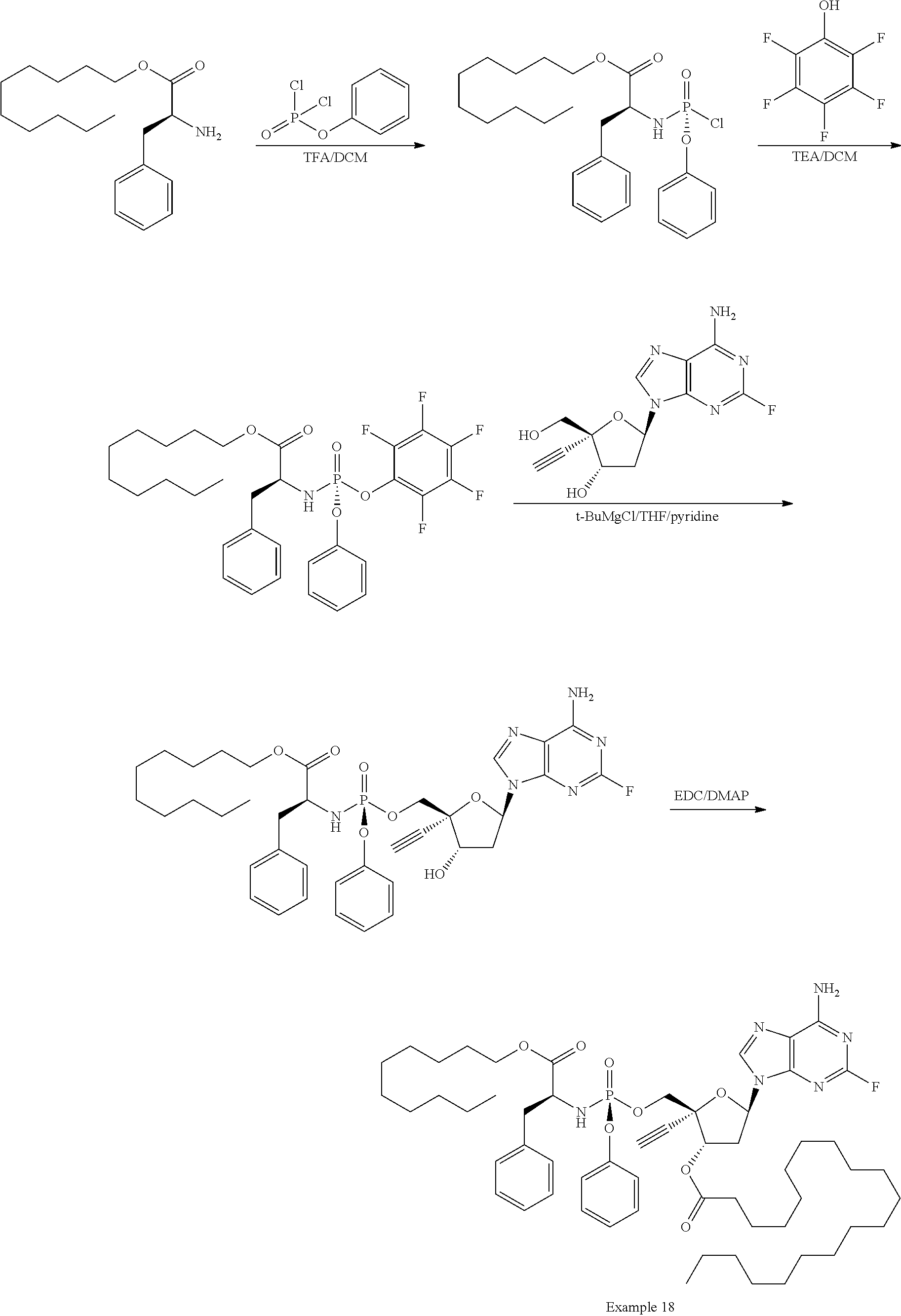

### Step 1: Decyl ((S)-(perfluorophenoxy)(phenoxy)phosphoryl)-L-phenylalaninate

To a solution of decyl L-phenylalaninate (3 g, 9.82 mmol) in DCM (20 mL) was added dropwise triethylamine (1.502 mL, 10.80 mmol) at −70° C. To this mixture was added a solution of phenyl phosphorodichloridate (2.051 g, 9.72 mmol) in anhydrous DCM (8 mL) dropwise. The reaction mixture was stirred at this temperature for additional 30 min and then allowed to warm to 0° C. over 2 h and stirred for 1 h. To this mixture was added dropwise a solution of 2,3,4,5,6-pentafluorophenol (1.790 g, 9.72 mmol) and triethylamine (1.502 mL, 10.80 mmol) in DCM (30 mL) and stirred at 0° C. for 2 h. LCMS showed the reaction was completed. The white solid (triethylaminehydrochloride) was filtered off and washed with DCM (10 mL). The filtrate was concentrated under reduced pressure, the residue was triturated with tert-butylmethyl ether (20 mL), and the triethylaminehydrochloride salt was removed by filtration. The cake was washed with tert-butylmethyl ether (2×10 mL), and the filtrate was concentrated under reduced pressure. The residue was triturated with 10% EtOAc/hexanes (30 mL) and solids collected by filtration to give decyl ((S)-(perfluorophenoxy)(phenoxy)phosphoryl)-L-phenylalaninate (1.5 g, 2.390 mmol, 24.34% yield) as white solid (>98% de as determined by ³¹PNMR). LCMS (M+H)=628.0; Retention time (0.1% TFA)=2.78 min. ³¹P NMR (DMSO-d₆, 162 MHz) δ 0.22.

### Step 2: Decyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a solution of (2R,3S,5R)-5-(6-amino-2-fluoro-9H-purin-9-yl)-2-ethynyl-2-(hydroxymethyl)tetrahydrofuran-3-ol (150 mg, 0.511 mmol) in anhydrous THF (30 mL) and pyridine (15 mL) was added dropwise tert-butylmagnesium chloride (1.023 mL, 1.023 mmol) at −15° C. The reaction mixture was stirred at the same temperature for 1 h. Then, a solution of decyl ((S)-(perfluorophenoxy)(phenoxy)phosphoryl)-L-phenylalaninate (642 mg, 1.023 mmol) in anhydrous THF (15 mL) was added dropwise at −15° C. and stirred at −15° C. for 16 h. LCMS showed the reaction was completed. The reaction mixture was quenched with 2N NH₄Cl (20 mL) and diluted with EtOAc (200 mL). The organic layer was washed with water (40 mL), brine (40 mL), dried over Na₂SO₄, filtered and concentrated. The residue was purified by reverse phase chromatography (SepaFlash@ C18 column, BOSTON, 40 g, 0-100% CH₃OH/10 mM NH₄HCO₃H2O) to give decyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.196 mmol, 38.4% yield) as white solid. LCMS (M+H)=737.0; Retention time (10 mM NH₄HCO₃)=2.20 min.

### Step 3: (2R,3S,5R)-5-(6-Amino-2-fluoro-9H-purin-9-yl)-2-((((S)-(((S)-1-(decyloxy)-1-oxo-3-phenylpropan-2-yl)amino)(phenoxy)phosphoryl)oxy)methyl)-2-ethynyltetrahydrofuran-3-yl icosanoate

To a stirred cold (ice-water bath) solution of icosanoic acid (63.6 mg, 0.204 mmol) in DCM (10 mL) were added 3-(((ethylimino)methylene)amino)-N,N-dimethylpropan-1-amine hydrochloride (195 mg, 1.018 mmol) and N,N-dimethylpyridin-4-amine (124 mg, 1.018 mmol) and stirred for 0.5 h. Then, to this mixture was added decyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.204 mmol) and stirred at 25° C. for 16 h. TLC showed the reaction was completed. The reaction mixture was partitioned between DCM (100 mL) and H₂O (30 mL). Then, the organic layer was washed with brine (20 mL), dried over Na₂SO₄, filtered and concentrated. The residue was purified by pre-TLC (DCM/CH₃OH=20/1) to give (2R,3S,5R)-5-(6-amino-2-fluoro-9H-purin-9-yl)-2-((((S)-(((S)-1-(decyloxy)-1-oxo-3-phenylpropan-2-yl)amino)(phenoxy)phosphoryl)oxy)methyl)-2-ethynyltetrahydrofuran-3-yl icosanoate (133 mg, 0.127 mmol, 62.5% yield) as colorless oil. HPLC Retention time (10 mM NH₄HCO₃)=19.81 min. ¹H NMR (400 MHz, CDCl₃) δ 8.02 (s, 1H), 7.26-7.14 (m, 7H), 7.13-7.09 (m, 3H), 6.40 (t, J=6.8 Hz, 1H), 6.11 (brs, 2H), 5.56 (dd, J=6.4, 4.4 Hz, 1H), 4.36-4.29 (m, 1H), 4.25 (dd, J=11.2, 6.0 Hz, 1H), 4.13 (dd, J=10.8, 5.6 Hz, 1H), 4.02 (t, J=6.8 Hz, 2H), 3.69 (t, J=10.8 Hz, 1H), 3.00 (d, J=6.4 Hz, 2H), 2.67-2.55 (m, 3H), 2.40 (t, J=7.6 Hz, 2H), 1.67 (quint, J=7.2 Hz, 2H), 1.52 (quint, J=7.2 Hz, 2H), 1.31-1.20 (m, 46H), 0.88 (t, J=6.8 Hz, 6H).

### Example 19

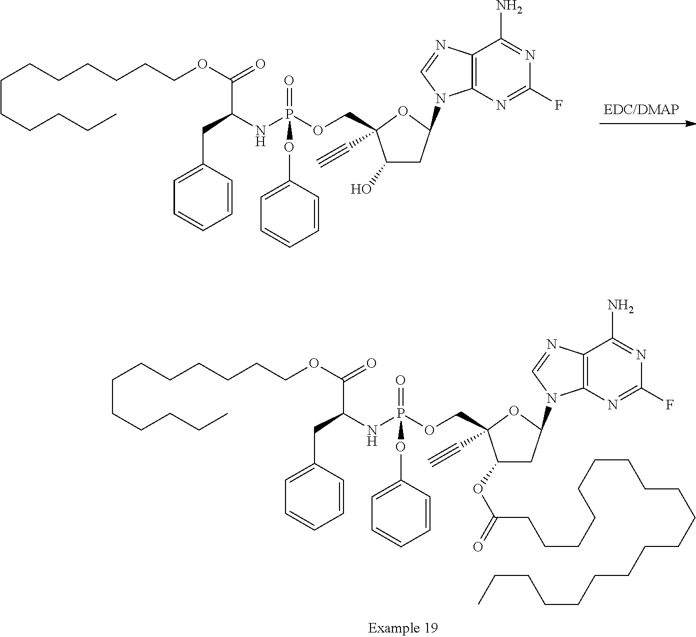 [SMILES: C#C[C@]1(CO[P@@](=O)(N[C@@H](Cc2ccccc2)C(=O)OCCCCCCCCCCCC)Oc2ccccc2)O[C@@H](n2cnc3c(N)nc(F)nc32)C[C@@H]1O ; C#C[C@]1(CO[P@@](=O)(N[C@@H](Cc2ccccc2)C(=O)OCCCCCCCCCCCC)Oc2ccccc2)O[C@@H](n2cnc3c(N)nc(F)nc32)C[C@@H]1OC(=O)CCCCCCCCCCCCCCCCCCC]

### (2R,3S,5R)-5-(6-Amino-2-fluoro-9H-purin-9-yl)-2-((((S)-(((S)-1-(dodecyloxy)-1-oxo-3-phenylpropan-2-yl)amino)(phenoxy)phosphoryl)oxy)methyl)-2-ethynyltetrahydrofuran-3-yl icosanoate

To a stirred cold (ice-water bath) solution of icosanoic acid (57.2 mg, 0.183 mmol) in DCM (15 mL) was added 3-(((ethylimino)methylene)amino)-N,N-dimethylpropan-1-amine hydrochloride (175 mg, 0.915 mmol) and N,N-dimethylpyridin-4-amine (112 mg, 0.915 mmol) and the resulting mixture was stirred for 0.5 h. Then, dodecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (N76670-81, 140 mg, 0.183 mmol) was added and stirred at room temperature for 4 h. TLC showed the reaction was completed. The reaction mixture was partitioned between DCM (100 mL) and H₂O (30 mL). The organic layer was washed with brine, dried over Na₂SO₄, filtered and concentrated. The residue was purified prep-TLC (DCM/CH₃OH=20/1) to give (2R,3S,5R)-5-(6-amino-2-fluoro-9H-purin-9-yl)-2-((((S)-(((S)-1-(dodecyloxy)-1-oxo-3-phenylpropan-2-yl)amino)(phenoxy)phosphor-yl)oxy)methyl)-2-ethynyltetrahydrofuran-3-yl icosanoate (103 mg, 0.092 mmol, 50.4% yield) as colorless oil. HPLC Retention time (10 mM NH₄HCO₃)=26.12 min. ¹H NMR (400 MHz, CDCl₃) δ 8.02 (s, 1H), 7.29-7.27 (m, 1H), 7.25-7.19 (m, 4H), 7.18-7.14 (m, 2H), 7.13-7.09 (m, 3H), 6.40 (t, J=6.8 Hz, 1H), 5.99 (brs, 2H), 5.56 (dd, J=6.0, 4.4 Hz, 1H), 4.36-4.29 (m, 1H), 4.25 (dd, J=11.2, 6.4 Hz, 1H), 4.13 (dd, J=10.8, 5.6 Hz, 1H), 4.02 (t, J=6.8 Hz, 2H), 3.63 (t, J=10.4 Hz, 1H), 3.00 (d, J=6.8 Hz, 2H), 2.65-2.57 (m, 3H), 2.40 (t, J=7.6 Hz, 2H), 1.67 (quint, J=7.2 Hz, 2H), 1.52 (quint, J=6.0 Hz, 2H), 1.30-1.23 (m, 50H), 0.88 (t, J=6.8 Hz, 6H).

### Example 20

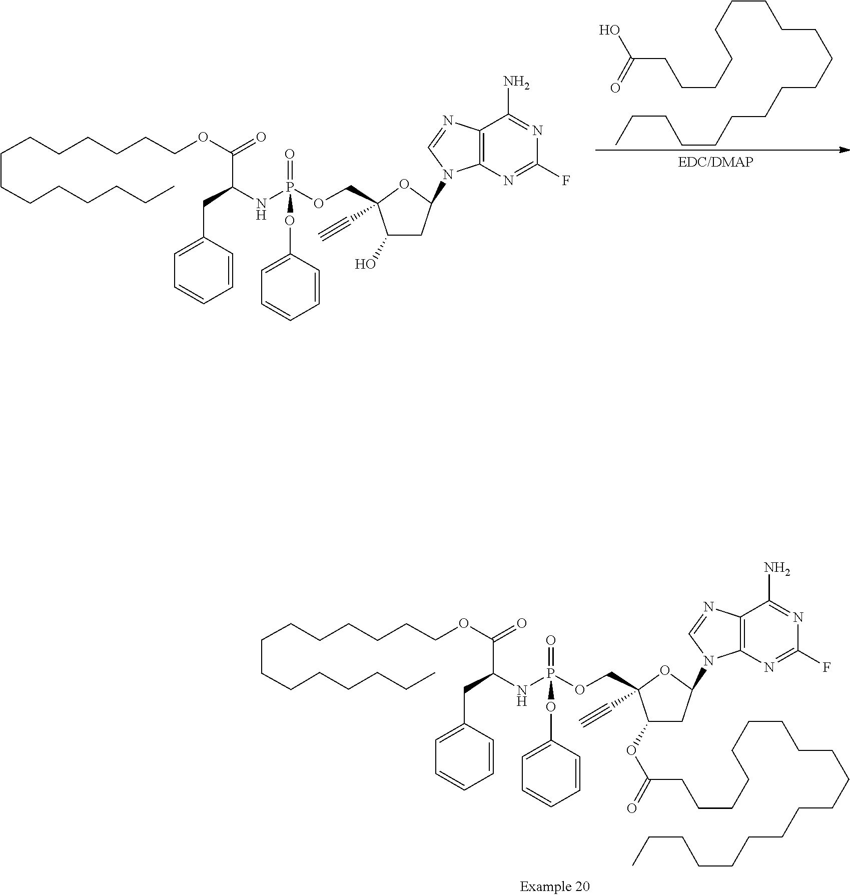 [SMILES: C#C[C@]1(CO[P@@](=O)(N[C@@H](Cc2ccccc2)C(=O)OCCCCCCCCCCCCCC)Oc2ccccc2)O[C@@H](n2cnc3c(N)nc(F)nc32)C[C@@H]1O ; CCCCCCCCCCCCCCCCCCCC(=O)O ; C#C[C@]1(CO[P@@](=O)(N[C@@H](Cc2ccccc2)C(=O)OCCCCCCCCCCCCCC)Oc2ccccc2)O[C@@H](n2cnc3c(N)nc(F)nc32)C[C@@H]1OC(=O)CCCCCCCCCCCCCCCCCCC]

### (2R,3S,5R)-5-(6-Amino-2-fluoro-9H-purin-9-yl)-2-ethynyl-2-((((S)-(((S)-1-oxo-3-phenyl-1-(tetradecyloxy)propan-2-yl)amino)(phenoxy)phosphoryl)oxy)methyl)tetrahydrofuran-3-yl icosanoate

To a stirred solution of icosanoic acid (87 mg, 0.277 mmol) in DCM (4 mL) was added DMAP (154 mg, 1.261 mmol) and EDC (242 mg, 1.261 mmol) at 0° C. and the resulting mixture was stirred for 0.5 h. Then, tetradecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (N77883-38, 200 mg, 0.252 mmol) in DCM (3 mL) was added at 0° C. The resulting mixture was stirred overnight at room temperature. TLC (DCM:CH₃OH=17:1, Rf=0.45) showed presence of new compound. The mixture was quenched with water (10 mL) and extracted with DCM (20 mL×3). The combined organic phases were washed with brine, dried over Na₂SO₄ and concentrated under vacuum. The residue was purified by prep-TLC (DCM:CH₃OH=17:1, Rf=0.45) to give (2R,3S,5R)-5-(6-amino-2-fluoro-9H-purin-9-yl)-2-ethynyl-2-((((S)-(((S)-1-oxo-3-phenyl-1-(tetradecyloxy)propan-2-yl)amino)(phenoxy)phosphoryl)oxy)methyl)tetrahydrofuran-3-yl icosanoate (172 mg, 0.149 mmol, 59.0% yield) as white solid. HPLC: Retention time (10 mM NH₄HCO₃): 37.405 min. ¹H NMR (400 MHz, CDCl₃) δ 8.03 (s, 1H), 7.25-7.21 (m, 4H), 7.19 (d, J=6.9 Hz, 1H), 7.15 (d, J=8.5 Hz, 2H), 7.08-7.03 (m, 3H), 6.39 (t, J=6.6 Hz, 1H), 6.04 (s, 2H), 5.56 (t, J=5.4 Hz, 1H), 4.36-4.28 (m, 1H), 4.25 (dd, J=11.1, 6.3 Hz, 1H), 4.13 (dd, J=11.1, 5.7 Hz, 1H), 4.02 (t, J=6.7 Hz, 2H), 3.64 (t, J=10.7 Hz, 1H), 3.00 (d, J=5.9 Hz, 2H), 2.64-2.54 (m, 3H), 2.39 (t, J=7.6 Hz, 2H), 1.71-1.62 (m, 2H), 1.55-1.48 (m, 2H), 1.28 (d, J=17.2 Hz, 54H), 0.88 (t, J=6.8 Hz, 6H).

### Example 21

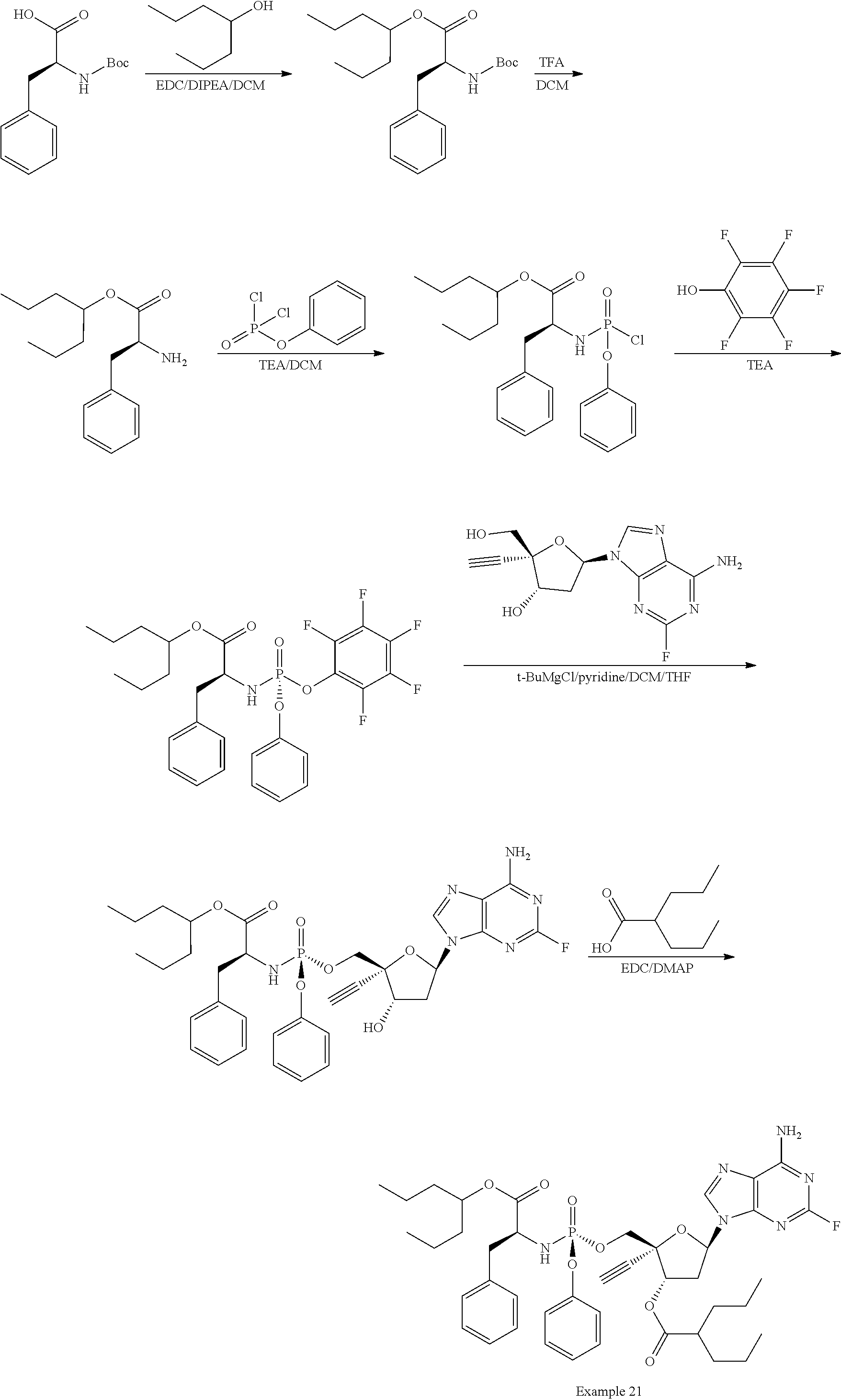

### Step: Heptan-4-yl (tert-butoxycarbonyl)-L-phenylalaninate

To a stirred mixture of (tert-butoxycarbonyl)-L-phenylalanine (40 g, 151 mmol) in DCM (300 mL) was added DMAP (1.842 g, 15.08 mmol) and EDC (37.6 g, 196 mmol) at 0° C. After 0.5 h, heptan-4-ol (22.80 g, 196 mmol) in DCM (50 mL) was added at 0° C. The mixture was stirred at room temperature for 2 h. LCMS showed the presence of new compound. The reaction mixture was diluted with water (90 mL), the organic layer was separated and the aqueous phase was extracted with DCM (100 mL×3). The combined organic phases were washed with brine, dried over Na₂SO₄ and concentrated. The residue was purified by silica gel chromatography (Santai, 220 g, pet. ether:EtOAc=30:1) to afford heptan-4-yl (tert-butoxycarbonyl)-L-phenylalaninate (45 g, 124 mmol, 82% yield) as light yellow oil. LCMS (M+Na)=386.1; Retention time (10% TFA)=2.36 min.

### Step 2: Heptan-4-yl L-phenylalaninate

To a stirred mixture of heptan-4-yl (tert-butoxycarbonyl)-L-phenylalaninate (45 g, 124 mmol) in DCM (300 mL) was added TFA (60 mL, 779 mmol) at 0° C., and the resulting mixture was stirred at 25° C. for 5 h. LCMS showed the reaction was completed. The reaction was concentrated and the pH of the residue was adjusted to 8-9 with NaOH (1 N) and extracted with DCM (100 mL×3). The combined organic phases were washed with brine, dried over Na₂SO₄ and concentrated. The residue was purified by silica gel chromatography (Santai, 220 g, DCM:MeOH=10: 1) to afford heptan-4-yl L-phenylalaninate (30 g, 114 mmol, 92% yield) as light yellow oil. LCMS (M+H)=264.0; time (0.1% TFA)=1.516 min.

### Step 3: Heptan-4-yl ((perfluorophenoxy)(phenoxy)phosphoryl)-L-phenylalaninate

To a solution of heptan-4-yl L-phenylalaninate (18.5 g, 70.2 mmol) in anhydrous DCM (35 mL) was added dropwise triethylamine (10.25 mL, 73.8 mmol) at −70° C. over 15 min. To this mixture was added a solution of phenyl phosphorodichloridate (14.67 g, 69.5 mmol) in anhydrous DCM (35 mL) over 1 h. The reaction mixture was stirred at this temperature for additional 30 min and then allowed to warm to 0° C. over 2 h and stirred for 1 h. To this mixture was added a solution of 2,3,4,5,6-pentafluorophenol (12.80 g, 69.5 mmol) and triethylamine (10.74 mL, 77 mmol) in DCM (30 mL) over 20 min and stirred at 0° C. for 4 h. LCMS showed the reaction was completed. The white solid (triethylamine hydrochloride) was filtered off and washed with DCM (25 mL). The filtrate was concentrated under reduced pressure, the residue was triturated with tert-butylmethyl ether (250 mL), and the triethylamine hydrochloride salt was removed by filtration. The cake was washed with tert-butylmethyl ether (2×50 mL), and the filtrate was concentrated under reduced pressure to give 44 g of crude product. The crude product was triturated with 5% EtOAc/hexanes (200 mL) and solids collected by filtration to give heptan-4-yl ((perfluorophenoxy)(phenoxy)phosphoryl)-L-phenylalaninate (5 g, 7.19 mmol, 10.24% yield) as white solid. (>98% de as determined by ³¹PNMR). LCMS (M+H)=586.2, Retention time (0.1% TFA)=2.137 min. ¹H NMR (400 MHz, CDCl₃) δ 7.33 (t, J=7.9 Hz, 2H), 7.23 (t, J=5.5 Hz, 3H), 7.21-7.15 (m, 3H), 7.12-7.08 (m, 2H), 4.91-4.83 (m, 1H), 4.42 (tt, J=9.8, 6.1 Hz, 1H), 3.59 (s, 1H), 3.10 (d, J=6.0 Hz, 2H), 1.50-1.39 (m, 4H), 1.26-1.16 (m, 4H), 0.84 (td, J=7.3, 5.3 Hz, 6H). ³¹P NMR (162 MHz, CDCl₃) δ−1.62.

### Step 2: Heptan-4-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (800 mg, 2.73 mmol) in THF (160 mL) and pyridine (20 mL) was added dropwise tert-butylmagnesium chloride (5.46 mL, 5.46 mmol) at −15° C. The reaction mixture was stirred at the same temperature for 1 h. Then, to this reaction mixture was added dropwise a solution of heptan-4-yl ((S)-(perfluorophenoxy)(phenoxy)phosphoryl)-L-phenylalaninate (2396 mg, 4.09 mmol) in anhydrous THF (10 mL) and stirred at −15° C. for 4 h. LCMS showed the reaction was completed. The reaction mixture was quenched with 2N NH₄Cl (20 mL) and partitioned between EtOAc (200 mL) and water (50 mL). The aqueous layer was extracted with EtOAc (2×150 mL) and the combined organic layers were washed with brine (50 mL), dried over Na₂SO₄, filtered and concentrated. The residue was purified by reverse phase chromatography (SepaFlash@ C18 column, BOSTON, 120 g, 0-100% CH₃CN/10 mM NH₄HCO₃H2O) to give heptan-4-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (700 mg, 0.984 mmol, 36.1% yield) as a white solid. LCMS (M+H)=695.3; Retention time (0.05% TFA)=1.78 min.

### Step 5: (2R,3S,5R)-5-(6-Amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl 2-propylpentanoate

To a stirred solution of 2-propylpentanoic acid (37.7 mg, 0.261 mmol) in DCM (4 mL) was added DMAP (145 mg, 1.188 mmol) and EDC (228 mg, 1.188 mmol) at 0° C. After 0.5 h, heptan-4-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (165 mg, 0.238 mmol) was added and the resulting mixture was stirred for 16 h at 15° C. The LCMS showed the reaction was completed. The mixture was diluted with water (20 mL), extracted with DCM (20 mL×2). The combined organic phases were washed with brine (20 mL), dried over Na₂SO₄, and concentrated under vacuum. The residue was purified by reverse phase chromatography (SepaFlash@ C18 column, BOSTON, 12 g, 0-100%, CH₃CN/10 mM NH₄HCO₃H2O) to give (2R,3S,5R)-5-(6-amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl 2-propylpentanoate (134 mg, 0.153 mmol, 64.5% yield) as white solid. LCMS (M+H)=821.7; Retention time (0.1% TFA)=2.396 min. HPLC: Retention time (10 mM NH₄HCO₃)=7.883 min. ¹H NMR (400 MHz, CDCl₃) δ 7.99 (s, 1H), 7.25-7.21 (m, 4H), 7.20-7.17 (m, 1H), 7.17-7.13 (m, 4H), 7.10 (t, J=7.3 Hz, 1H), 6.38 (t, J=6.6 Hz, 1H), 5.98 (s, 2H), 5.53 (dd, J=7.0, 4.7 Hz, 1H), 4.87-4.80 (m, 1H), 4.33 (tt, J=10.1, 6.7 Hz, 1H), 4.20 (dd, J=11.1, 6.2 Hz, 1H), 4.05 (dd, J=11.1, 5.6 Hz, 1H), 3.65 (t, J=10.5 Hz, 1H), 3.06 (dd, J=13.8, 6.2 Hz, 1H), 2.94 (dd, J=13.8, 6.9 Hz, 1H), 2.73-2.64 (m, 1H), 2.61 (s, 1H), 2.57 (ddd, J=13.9, 6.4, 4.7 Hz, 1H), 2.47 (tt, J=8.6, 5.5 Hz, 1H), 1.70-1.60 (m, 2H), 1.52-1.45 (m, 2H), 1.45-1.38 (m, 4H), 1.34 (dd, J=15.0, 7.4 Hz, 4H), 1.23-1.12 (m, 4H), 0.92 (td, J=7.2, 1.6 Hz, 6H), 0.83 (td, J=7.3, 1.5 Hz, 6H).

### Example 22

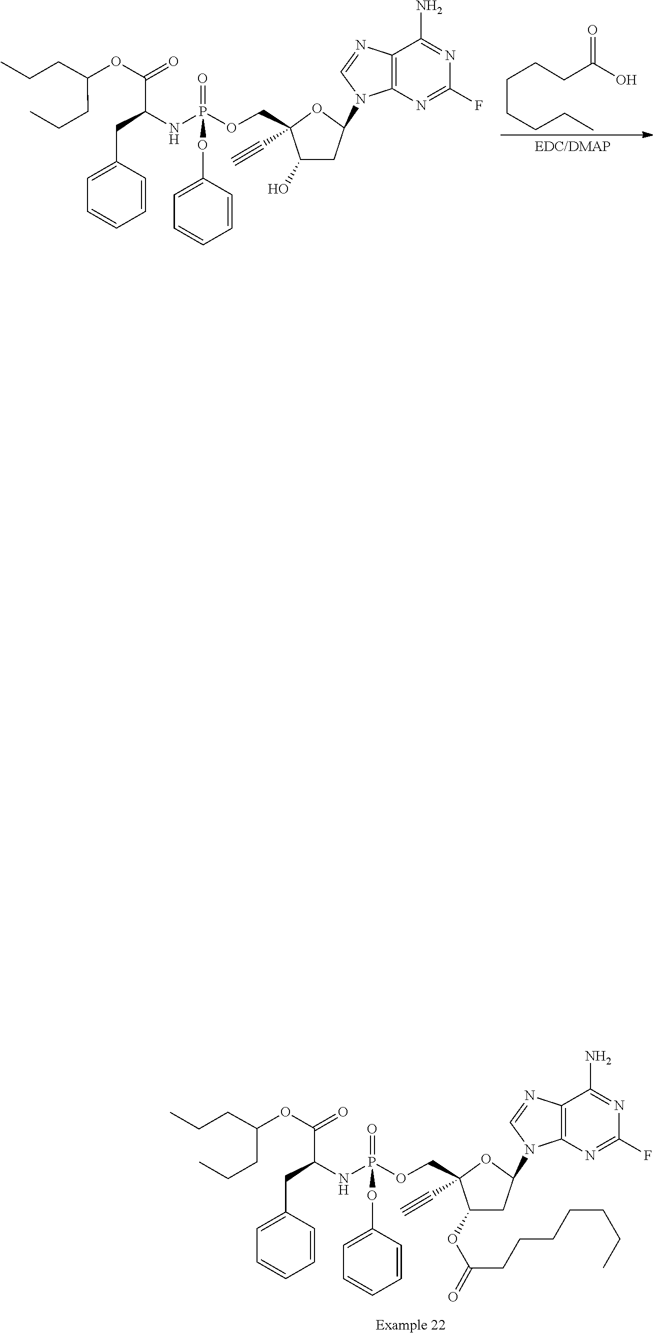

### (2R,3S,5R)-5-(6-Amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl octanoate

To a stirred cold (ice-water bath) solution of octanoic acid (37.4 mg, 0.259 mmol) in DCM (10 mL) was added 3-(((ethylimino)methylene)amino)-N,N-dimethylpropan-1-amine hydrochloride (207 mg, 1.080 mmol) and N,N-dimethylpyridin-4-amine (132 mg, 1.080 mmol) and stirred for 0.5 h. Then, heptan-4-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.216 mmol) was added and stirred at 25° C. for 4 h. LCMS showed the reaction was completed. The reaction mixture was partitioned between DCM (100 mL) and H₂O (30 mL). The organic layer was washed with brine (30 mL), dried over Na₂SO₄, filtered and concentrated. The residue was purified by reverse phase chromatography (SepaFlash@ C18 column, BOSTON, 40 g, 0-100% CH₃CN/10 mM NH₄HCO₃H2O) to give (2R,3S,5R)-5-(6-amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl octanoate (107 mg, 0.124 mmol, 57.5% yield) as white solid. LCMS (M+H)=821.3; Retention time (0.05% TFA)=2.14 min. HPLC Retention time (10 mM NH₄HCO₃)=8.09 min. ¹H NMR (400 MHz, CDCl₃) δ 8.00 (s, 1H), 7.26-7.21 (m, 4H), 7.19-7.09 (m, 6H), 6.39 (t, J=6.8 Hz, 1H), 6.24 (brs, 2H), 5.55 (t, J=5.2 Hz, 1H), 4.90-4.82 (m, 1H), 4.38-4.28 (m, 1H), 4.19 (dd, J=10.8, 6.4 Hz, 1H), 4.06 (dd, J=11.2, 5.6 Hz, 1H), 3.75 (t, J=10.8 Hz, 1H), 3.07 (dd, J=14.0, 6.4 Hz, 1H), 2.94 (dd, J=14.0, 7.2 Hz, 1H), 2.62 (s, 1H), 2.60-2.55 (m, 2H), 2.40 (t, J=7.6 Hz, 2H), 1.67 (quint, J=7.2 Hz, 2H), 1.43-1.19 (m, 16H), 0.89 (t, J=6.8 Hz, 3H), 0.83 (dt, J=7.2, 2.0 Hz, 6H).

### Example 23

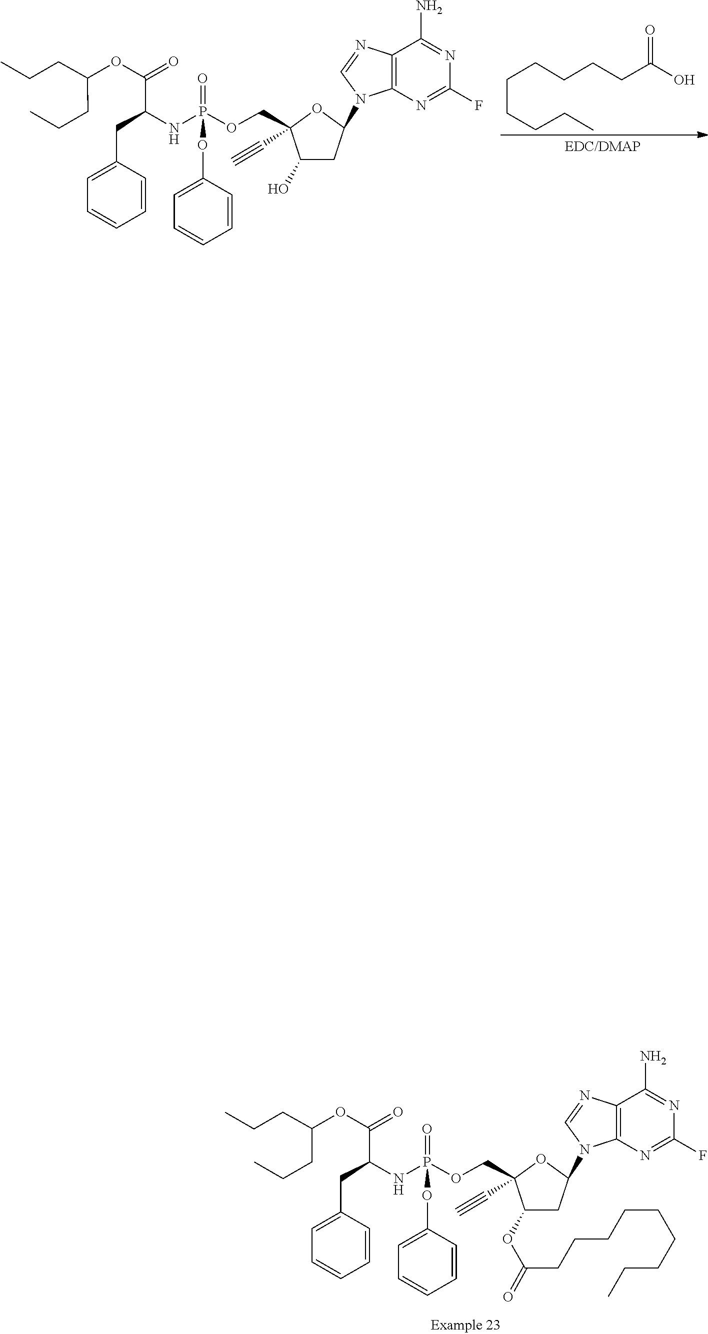

### (2R,3S,5R)-5-(6-Amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl decanoate

To a stirred cold (ice-water bath) solution of decanoic acid (44.6 mg, 0.259 mmol) in DCM (10 mL) was added N,N-dimethylpyridin-4-amine (132 mg, 1.080 mmol) and 3-(((ethylimino)methylene)amino)-N,N-dimethylpropan-1-amine hydrochloride (207 mg, 1.080 mmol) and the resulting mixture was stirred for 0.5 h. Then, heptan-4-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.216 mmol) was added and the resulting mixture was stirred at 25° C. for 4 h. The LCMS showed the reaction was completed. The reaction mixture was partitioned between DCM (100 mL) and H₂O (30 mL). The organic layer was washed with brine (20 mL), dried over Na₂SO₄, filtered and concentrated. The residue was purified by reverse phase chromatography (SepaFlash@ C18 column, BOSTON, 40 g, 0-100% CH₃CN/10 mM NH₄HCO₃H2O) to give (2R,3S,5R)-5-(6-amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl decanoate (150 mg, 0.175 mmol, 81% yield) as colorless oil. LCMS (M+H)=849.3; Retention time (0.05% TFA)=2.28 min; HPLC Retention time (10 mM NH₄HCO₃)=8.72 min. ¹H NMR (400 MHz, CDCl₃) δ 8.00 (s, 1H), 7.23 (dd, J=7.6, 2.4 Hz, 4H), 7.20-7.09 (m, 6H), 6.39 (t, J=6.8 Hz, 1H), 6.00 (brs, 2H), 5.57-5.53 (m, 1H), 4.90-4.81 (m, 1H), 4.38-4.28 (m, 1H), 4.19 (dd, J=11.2, 6.4 Hz, 1H), 4.05 (dd, J=10.8, 5.6 Hz, 1H), 3.63 (t, J=10.8 Hz, 1H), 3.07 (dd, J=13.6, 6.4 Hz, 1H), 2.94 (dd, J=14.4, 7.2 Hz, 1H), 2.62 (s, 1H), 2.61-2.55 (m, 2H), 2.40 (t, J=7.6 Hz, 2H), 1.67 (quint, J=7.2 Hz, 2H), 1.48-1.39 (m, 4H), 1.34-1.25 (m, 12H), 1.24-1.15 (m, 4H), 0.90-0.80 (m, 9H).

### Example 24

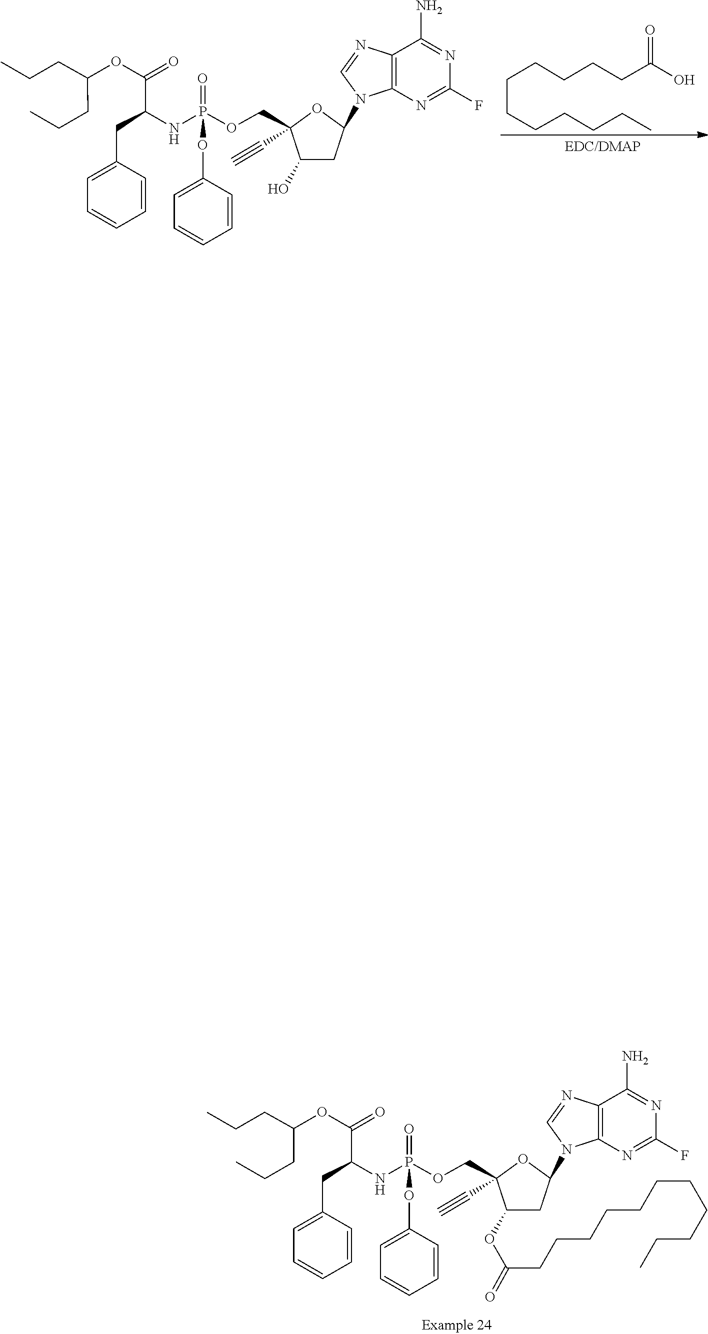

### (2R,3S,5R)-5-(6-Amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl dodecanoate

To a stirred solution of dodecanoic acid (47.6 mg, 0.238 mmol) in DCM (4 mL) was added DMAP (132 mg, 1.080 mmol) and EDC (207 mg, 1.080 mmol) at 0° C. After 0.5 h, heptan-4-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.216 mmol) in DCM (2 mL) was added. The resulting mixture was stirred overnight at 15° C. The LCMS showed the reaction was completed. The reaction mixture was diluted with water (20 mL), the organic layer was separated and the aqueous phase was extracted with DCM (20 mL×2). The combined organic phases were washed with brine (20 mL), dried over Na₂SO₄ and concentrated under vacuum. The residue was purified by reverse phase chromatography (SepaFlash@ C18 column, BOSTON, 12 g, 0-100%, CH₃CN/10 mM NH₄HCO₃H2O) to give (2R,3S,5R)-5-(6-amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl dodecanoate (103 mg, 0.116 mmol, 53.5% yield) as white solid. LCMS (M+H)=877.7; Retention time (0.1% TFA)=2.837 min. HPLC: Retention time (10 mM NH₄HCO₃)=9.728 min. ¹H NMR (400 MHz, CDCl₃) δ 8.03 (s, 1H), 7.25-7.21 (m, 4H), 7.19 (d, J=7.0 Hz, 1H), 7.17-7.13 (m, 4H), 7.10 (t, J=7.3 Hz, 1H), 6.39 (t, J=6.8 Hz, 1H), 6.06 (s, 2H), 5.55 (t, J=5.4 Hz, 1H), 4.88-4.81 (m, 1H), 4.33 (tt, J=10.1, 6.7 Hz, 1H), 4.19 (dd, J=11.1, 6.5 Hz, 1H), 4.05 (dd, J=11.1, 5.8 Hz, 1H), 3.64 (t, J=10.6 Hz, 1H), 3.07 (dd, J=13.8, 5.9 Hz, 1H), 2.94 (dd, J=13.8, 7.0 Hz, 1H), 2.62 (s, 1H), 2.57 (t, J=6.3 Hz, 2H), 2.40 (t, J=7.6 Hz, 2H), 1.71-1.62 (m, 2H), 1.47-1.38 (m, 4H), 1.33-1.24 (m, 16H), 1.18 (ddd, J=11.7, 9.8, 6.1 Hz, 4H), 0.88 (t, J=6.9 Hz, 3H), 0.83 (td, J=7.3, 2.5 Hz, 6H).

### Example 25

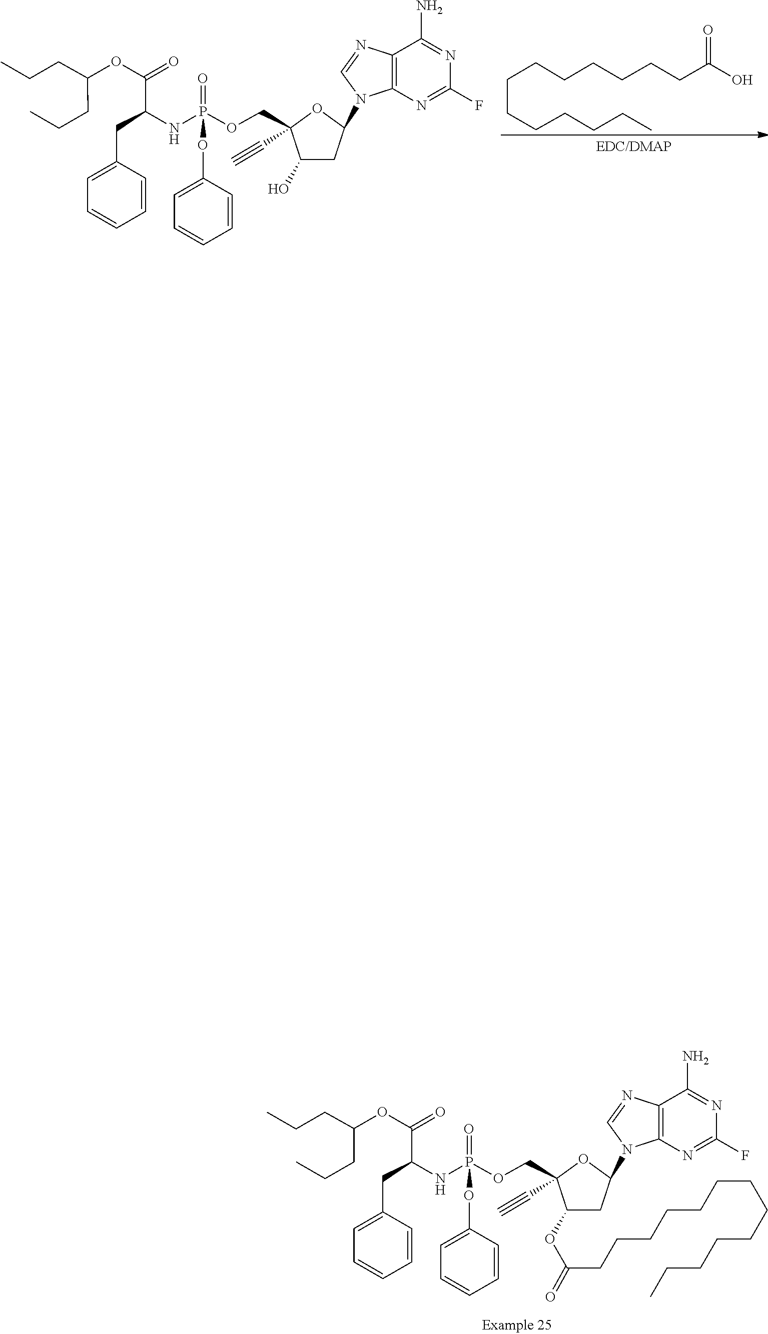

### (2R,3S,5R)-5-(6-Amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl tetradecanoate

To a stirred solution of tetradecanoic acid (54.2 mg, 0.238 mmol) in DCM (4 mL) was added DMAP (132 mg, 1.080 mmol) and EDC (207 mg, 1.080 mmol) at 0° C. After 0.5 h, heptan-4-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.216 mmol) in DCM (2 mL) was added. The resulting mixture was stirred overnight at 15° C. The LCMS showed the reaction was completed. The reaction mixture was diluted with water (20 mL), the organic layer was separated and the aqueous phase was extracted with DCM (20 mL×2). The combined organic phases were washed with brine (20 mL), dried over Na₂SO₄ and concentrated under vacuum. The crude product was purified by reverse phase chromatography (SepaFlash@ C18 column, BOSTON, 12 g, 0-100%, CH₃CN/10 mM NH₄HCO₃H2O) to (2R,3S,5R)-5-(6-amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl tetradecanoate (140 mg, 0.153 mmol, 71.0% yield) as white solid. LCMS (M+H)=905.1; Retention time (0.1% TFA)=3.237 min. HPLC: Retention time (10 mM NH₄HCO₃)=9.766 min. ¹H NMR (400 MHz, CDCl₃) δ 8.05 (s, 1H), 7.26-7.21 (m, 4H), 7.20-7.17 (m, 1H), 7.15 (dd, J=9.4, 4.9 Hz, 4H), 7.10 (t, J=7.3 Hz, 1H), 6.39 (t, J=6.7 Hz, 1H), 6.15 (s, 2H), 5.54 (t, J=5.4 Hz, 1H), 4.89-4.81 (m, 1H), 4.33 (ddd, J=16.6, 10.0, 6.8 Hz, 1H), 4.18 (dd, J=11.1, 6.6 Hz, 1H), 4.06 (dd, J=11.1, 5.8 Hz, 1H), 3.67 (s, 1H), 3.07 (dd, J=13.7, 6.1 Hz, 1H), 2.94 (dd, J=13.8, 7.1 Hz, 1H), 2.62 (s, 1H), 2.59-2.53 (m, 2H), 2.40 (t, J=7.6 Hz, 2H), 1.66 (dd, J=14.6, 7.2 Hz, 2H), 1.47-1.38 (m, 4H), 1.32 (s, 2H), 1.26 (s, 18H), 1.23-1.14 (m, 4H), 0.85 (ddd, J=11.2, 9.6, 4.4 Hz, 9H).

### Example 26

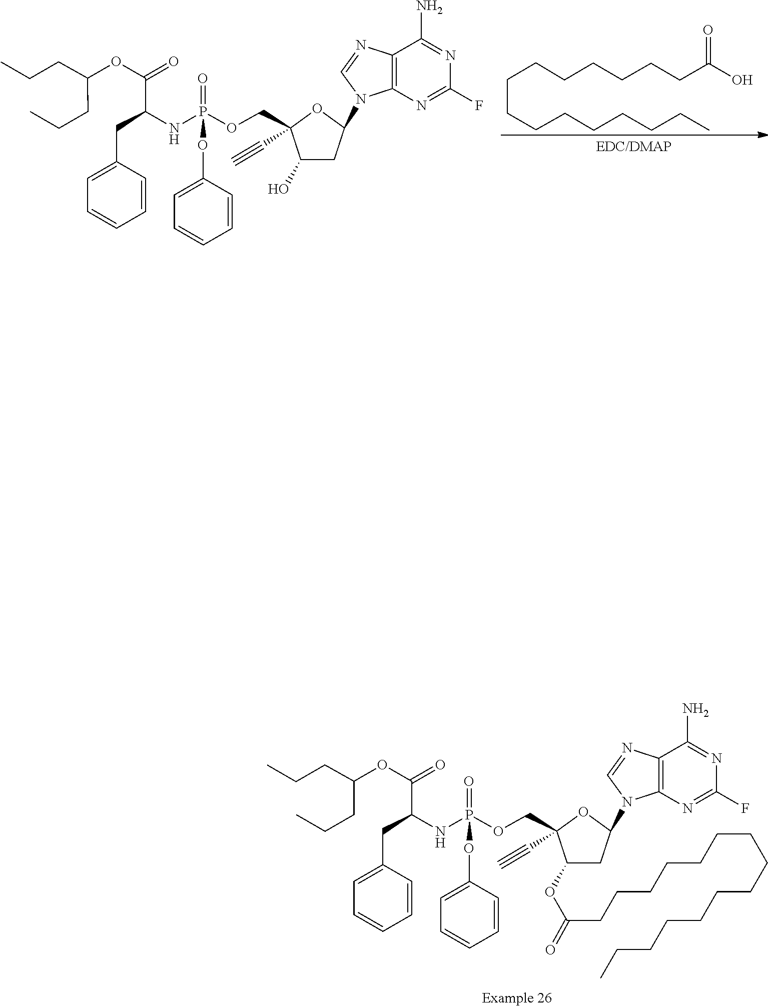

### (2R,3S,5R)-5-(6-Amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl palmitate

To a stirred cold (ice-water bath) solution of palmitic acid (72.0 mg, 0.281 mmol) in DCM (10 mL) was added N,N-dimethylpyridin-4-amine (132 mg, 1.080 mmol) and 3-(((ethylimino)methylene)amino)-N,N-dimethylpropan-1-amine hydrochloride (207 mg, 1.080 mmol) and the resulting mixture was stirred for 0.5 h. Then, heptan-4-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.216 mmol) was added and the resulting mixture was stirred at 25° C. for 4 h. The LCMS showed the reaction was completed. The reaction mixture was partitioned between DCM (80 mL) and H₂O (20 mL). The organic layer was washed with brine (20 mL), dried over Na₂SO₄, filtered and concentrated. The residue was purified by reverse phase chromatography (SepaFlash@ C18 column, BOSTON, 40 g, 0-100% MeOH/10 mM NH₄HCO₃H2O) to give (2R,3S,5R)-5-(6-amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl palmitate (161 mg, 0.166 mmol, 77% yield) as light yellow semi-solid. LCMS (M+H)=933.5; Retention time (0.05% TFA)=3.79 min. HPLC Retention time (10 mM NH₄HCO₃)=12.41 min. ¹H NMR (400 MHz, CDCl₃) δ 8.00 (s, 1H), 7.23 (dd, J=7.6, 2.4 Hz, 4H), 7.21-7.18 (m, 1H), 7.18-7.13 (m, 4H), 7.10 (t, J=7.2 Hz, 1H), 6.39 (t, J=6.8 Hz, 1H), 6.01 (brs, 2H), 5.58-5.53 (m, 1H), 4.85 (quint, J=6.0 Hz, 1H), 4.38-4.28 (m, 1H), 4.19 (dd, J=11.2, 6.4 Hz, 1H), 4.05 (dd, J=11.2, 5.6 Hz, 1H), 3.64 (t, J=10.4 Hz, 1H), 3.07 (dd, J=13.2, 5.6 Hz, 1H), 2.94 (dd, J=14.0, 7.2 Hz, 1H), 2.62 (s, 1H), 2.61-2.55 (m, 2H), 2.40 (t, J=7.6 Hz, 2H), 1.67 (quint, J=6.8 Hz, 2H), 1.48-1.39 (m, 4H), 1.34-1.26 (m, 24H), 1.22-1.12 (m, 4H), 0.88 (t, J=6.8 Hz, 3H), 0.83 (dt, J=7.2, 2.8 Hz, 6H).

### Example 27

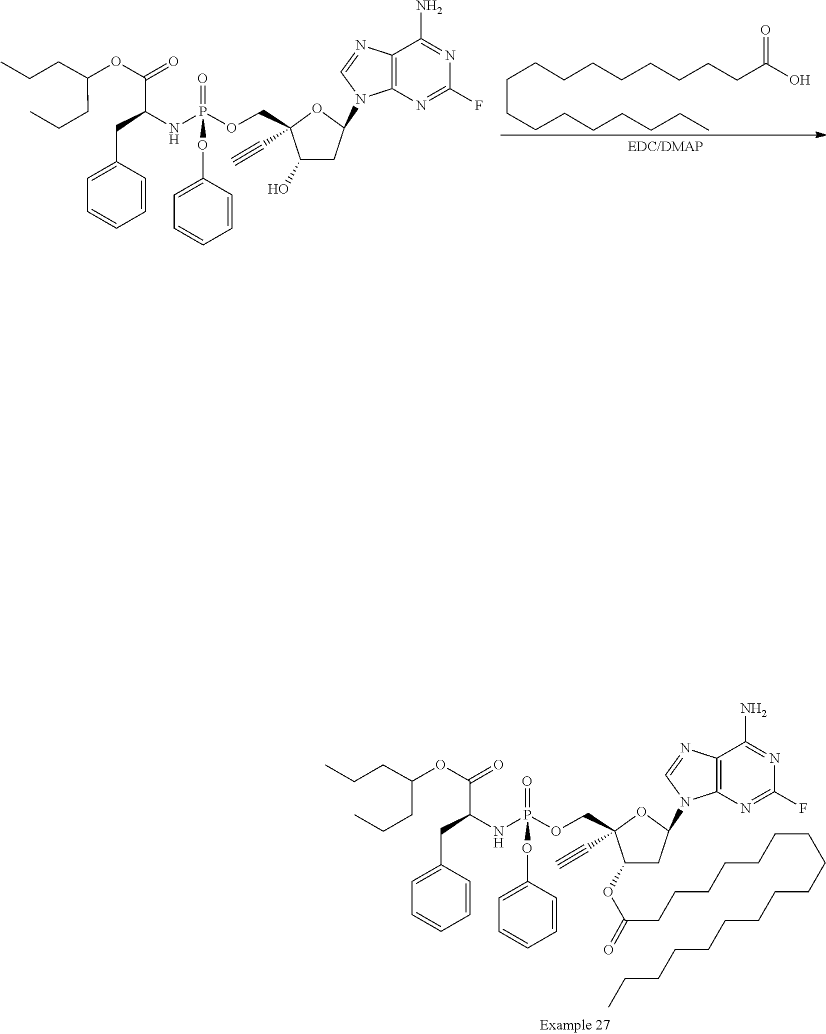

### (2R,3S,5R)-5-(6-Amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl stearate

To a stirred cold (ice-water bath) solution of stearic acid (133 mg, 0.468 mmol) in DCM (15 mL) were added N,N-dimethylpyridin-4-amine (220 mg, 1.799 mmol) and 3-(((ethylimino)methylene)amino)-N,N-dimethylpropan-1-amine hydrochloride (345 mg, 1.799 mmol) and the resulting mixture was stirred for 0.5 h. Then, heptan-4-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (250 mg, 0.360 mmol) was added. The resulting mixture was stirred at 25° C. for 4 h. The LCMS showed the reaction was completed. The reaction mixture was partitioned between DCM (100 mL) and H₂O (30 mL). The organic layer was washed with brine (20 mL), dried over Na₂SO₄, filtered and concentrated. The residue was purified by reverse phase chromatography (SepaFlash@ C18 column, BOSTON, 40 g, 0-100% MeOH/10 mM NH₄HCO₃H2O) to give (2R,3S,5R)-5-(6-amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl stearate (110 mg, 0.113 mmol, 31.3% yield) as light yellow solid. LCMS (M+H)=962.4; Retention time (0.05% TFA)=4.29 min. HPLC Retention time (10 mM NH₄HCO₃)=17.03 min. ¹H NMR (400 MHz, CDCl₃) δ 8.02 (s, 1H), 7.23 (dd, J=7.6, 2.0 Hz, 4H), 7.21-7.18 (m, 1H), 7.18-7.13 (m, 4H), 7.10 (t, J=7.2 Hz, 1H), 6.39 (t, J=6.8 Hz, 1H), 5.97 (brs, 2H), 5.55 (t, J=4.8 Hz, 1H), 4.85 (quint, J=5.6 Hz, 1H), 4.38-4.29 (m, 1H), 4.19 (dd, J=10.8, 6.4 Hz, 1H), 4.05 (dd, J=11.2, 6.0 Hz, 1H), 3.61 (t, J=10.4 Hz, 1H), 3.07 (dd, J=13.6, 6.4 Hz, 1H), 2.94 (dd, J=14.0, 7.2 Hz, 1H), 2.62 (s, 1H), 2.57 (t, J=6.4 Hz, 2H), 2.40 (t, J=7.2 Hz, 2H), 1.67 (quint, J=7.2 Hz, 2H), 1.49-1.39 (m, 4H), 1.29-1.22 (m, 28H), 1.21-1.10 (m, 4H), 0.88 (t, J=6.4 Hz, 3H), 0.83 (dt, J=7.6, 2.8 Hz, 6H).

### Example 28

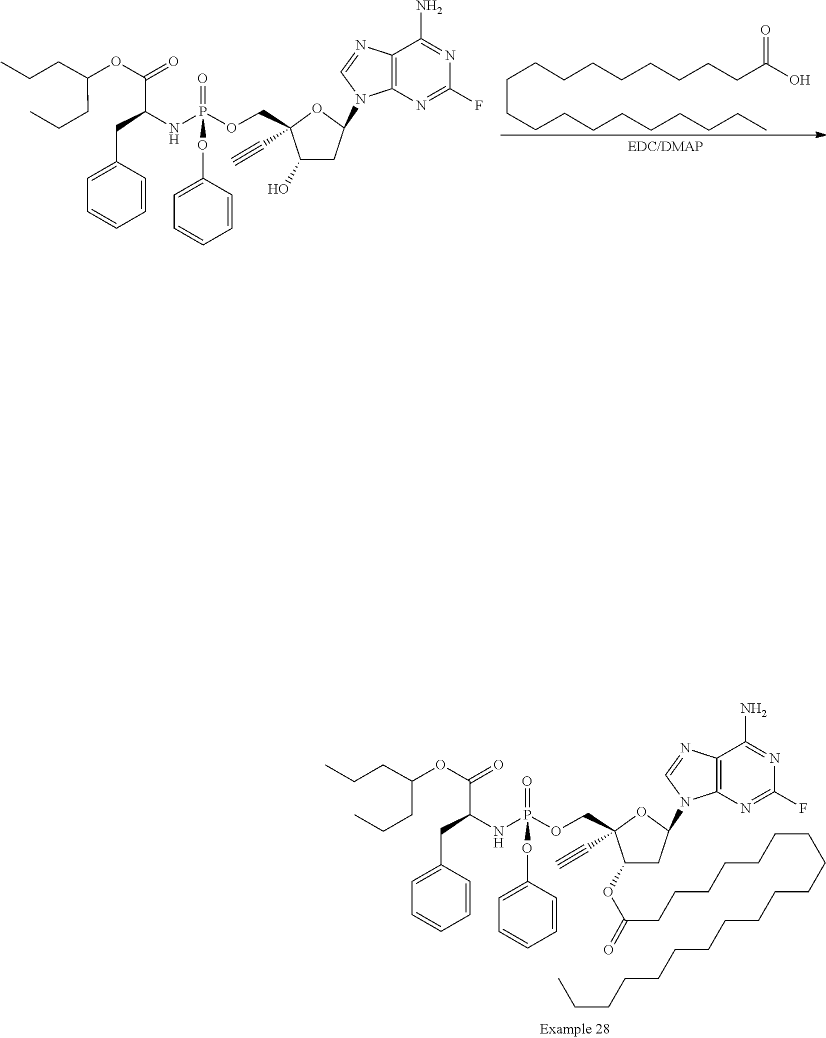

### (2R,3S,5R)-5-(6-Amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl icosanoate

To a stirred solution of icosanoic acid (82 mg, 0.261 mmol) in DCM (4 mL) was added DMAP (145 mg, 1.188 mmol) and EDC (228 mg, 1.188 mmol) at 0° C. After 0.5 h, heptan-4-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (165 mg, 0.238 mmol) in DCM (2 mL) was added. The resulting mixture was stirred overnight at 15° C. TLC (DCM:MeOH=20:1, R_{f}=0.3) showed presence of new compound. The reaction mixture was diluted with water (20 mL), the organic layer was separated and the aqueous layer was extracted with DCM (20 mL×2). The combined organic phases were washed with brine (30 mL), dried over Na₂SO₄ and concentrated under vacuum. The residue was purified by prep-TLC (DCM:MeOH=20:1, R_{f}=0.3) to give (2R,3S,5R)-5-(6-amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl icosanoate (143 mg, 0.138 mmol, 58.1% yield) as white solid. HPLC: Retention time (10 mM NH₄HCO₃)=13.594 min. ¹H NMR (400 MHz, CDCl₃) δ 8.10 (s, 1H), 7.24 (d, J=7.7 Hz, 4H), 7.19 (d, J=7.0 Hz, 1H), 7.17 (d, J=1.4 Hz, 1H), 7.14 (d, J=8.4 Hz, 3H), 7.10 (t, J=7.3 Hz, 1H), 6.38 (t, J=6.7 Hz, 1H), 6.04 (s, 2H), 5.54 (t, J=5.3 Hz, 1H), 4.86 (dd, J=12.2, 5.8 Hz, 1H), 4.36-4.28 (m, 1H), 4.18 (dd, J=11.1, 6.6 Hz, 1H), 4.05 (dd, J=11.1, 6.0 Hz, 1H), 3.61 (t, J=10.7 Hz, 1H), 3.08 (dd, J=13.6, 5.7 Hz, 1H), 2.94 (dd, J=13.8, 7.1 Hz, 1H), 2.62 (s, 1H), 2.59-2.52 (m, 2H), 2.40 (t, J=7.6 Hz, 2H), 1.71-1.62 (m, 2H), 1.48-1.39 (m, 4H), 1.38-1.30 (m, 4H), 1.25 (s, 28H), 1.22-1.15 (m, 4H), 0.85 (ddd, J=10.4, 9.6, 4.4 Hz, 9H).

### Example 29

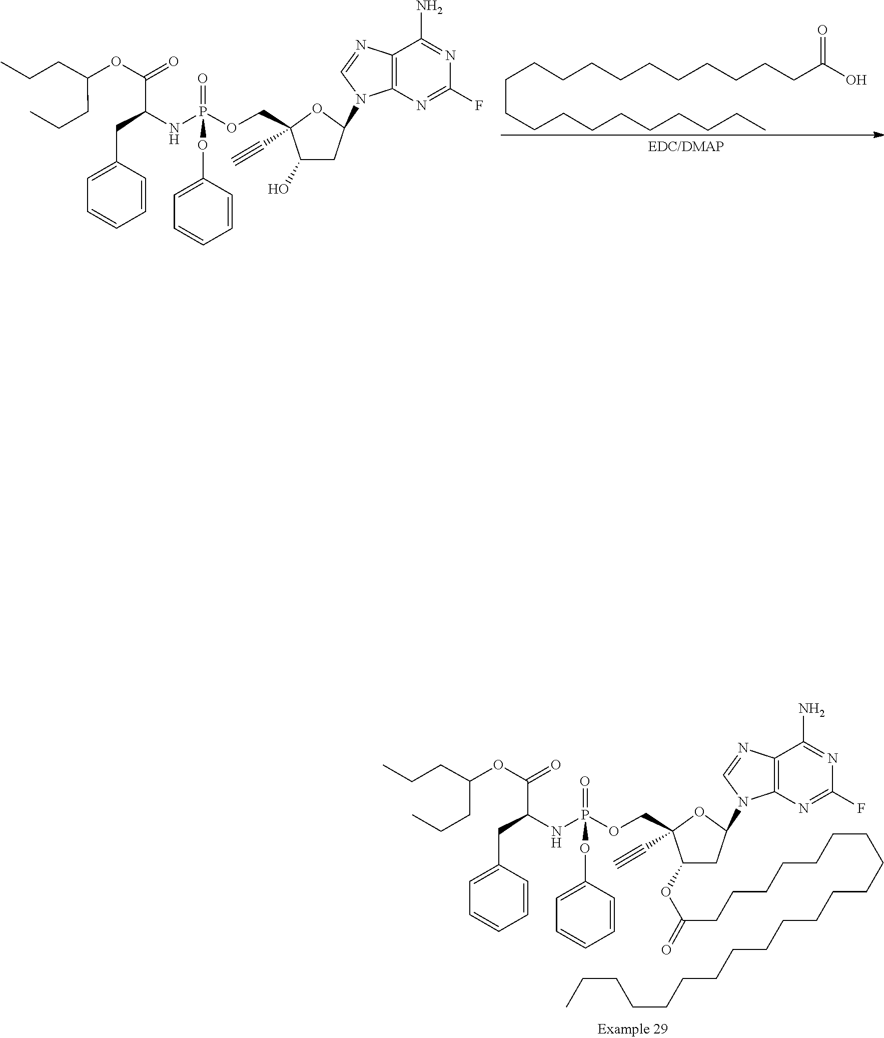

### (2R,3S,5R)-5-(6-Amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl docosanoate

To a stirred solution of docosanoic acid (89 mg, 0.261 mmol) in DCM (4 mL) was added DMAP (145 mg, 1.188 mmol) and EDC (228 mg, 1.188 mmol) at 0° C. After 0.5 h, heptan-4-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (165 mg, 0.238 mmol) was added at 0° C. The resulting mixture was stirred for 16 h at 15° C. TLC (DCM:MeOH=20:1, Rf=0.3) showed presence of new compound. The reaction mixture was diluted with water (10 mL), the organic layer was separated and the aqueous layer was extracted with DCM (20 mL×2). The combined organic phases were washed with brine (30 mL), dried over Na₂SO₄ and concentrated under vacuum. The mixture was purified by reverse phase chromatography (SepaFlash@ C18 column, BOSTON, 0-100% CH₃OH/10 mM NH₄HCO₃H2O) to afford (2R,3S,5R)-5-(6-amino-2-fluoro-9H-purin-9-yl)-2-ethynyl-2-((((S)-(((S)-1-(heptan-4-yloxy)-1-oxo-3-phenylpropan-2-yl)amino)(phenoxy)phosphoryl)oxy)methyl)tetrahydrofuran-3-yl docosanoate (107 mg, 0.104 mmol, 43.7% yield) as white solid. HPLC: Retention time (10 mM NH₄HCO₃)=17.825 min. ¹H NMR (400 MHz, CDCl₃) δ 8.01 (s, 1H), 7.25-7.20 (m, 4H), 7.18 (d, J=7.0 Hz, 1H), 7.17-7.12 (m, 4H), 7.09 (t, J=7.3 Hz, 1H), 6.39 (t, J=6.7 Hz, 1H), 6.19 (s, 2H), 5.55 (t, J=5.2 Hz, 1H), 4.84 (dd, J=11.9, 6.2 Hz, 1H), 4.33 (t, J=6.4 Hz, 1H), 4.19 (dd, J=11.0, 6.5 Hz, 1H), 4.06 (dd, J=11.0, 5.8 Hz, 1H), 3.71 (t, J=10.6 Hz, 1H), 3.07 (dd, J=13.7, 5.9 Hz, 1H), 2.94 (dd, J=13.7, 6.9 Hz, 1H), 2.61 (s, 1H), 2.56 (d, J=6.4 Hz, 2H), 2.39 (t, J=7.5 Hz, 2H), 1.66 (dd, J=14.4, 7.2 Hz, 2H), 1.46-1.38 (m, 4H), 1.30 (s, 2H), 1.25 (s, 34H), 1.21-1.14 (m, 4H), 0.84 (ddd, J=11.4, 9.2, 4.2 Hz, 9H).

### Example 30

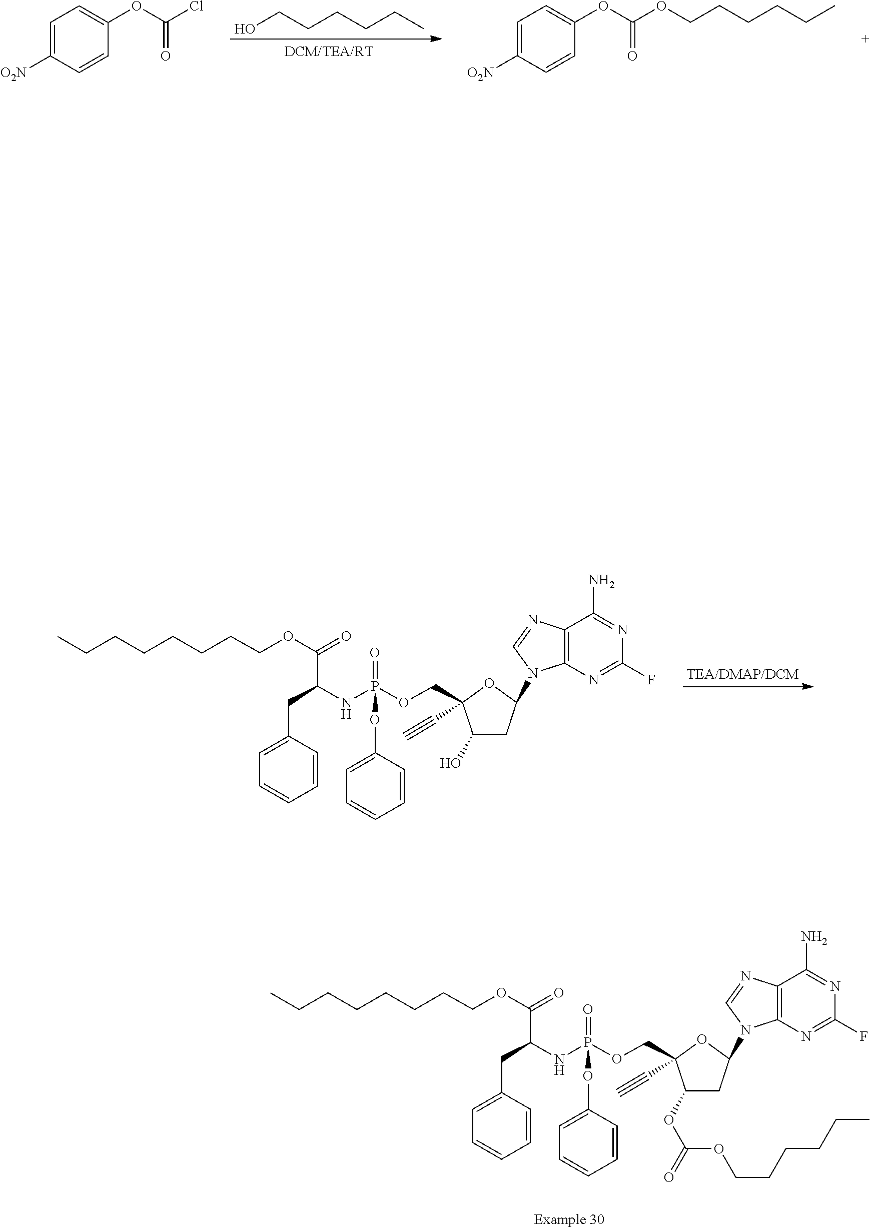

### Step 1: Hexyl (4-nitrophenyl) carbonate

To a mixture of 4-nitrophenyl carbonochloridate (230 g, 1141 mmol) and hexan-1-ol (117 g, 1141 mmol) in DCM (1000 mL) was added triethylamine (239 mL, 1712 mmol) and stirred at room temperature for 1 h. TLC indicated that the reaction was completed. The reaction mixture was concentrated to give crude hexyl (4-nitrophenyl) carbonate (280 g, 943 mmol, 103% yield) as yellow oil which was used in the next step without purification.

### Step 2: Octyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate

To a stirred mixture of octyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (160 mg, 0.226 mmol) in DCM (10 mL) was added triethylamine (0.094 mL, 0.677 mmol) followed by hexyl (4-nitrophenyl) carbonate (121 mg, 0.452 mmol) and stirred at room temperature for 2 days. TLC showed the new product formed. The residue was concentrated and purified by flash chromatography (silica gel, 0-10% MeOH/DCM) to give octyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate (160 mg, 0.187 mmol, 83% yield) as white foam. LCMS (M+H)=837.7; Retention time (0.1% TFA)=2.43 min. ¹HNMR (400 MHz, CDCl₃) δ 7.26-7.05 (m, 10H), 6.38 (t, J=6.7 Hz, 1H), 6.15 (s, 2H), 5.47 (dd, J=6.6, 4.5 Hz, 1H), 4.47-4.25 (m, 2H), 4.27-4.12 (m, 3H), 4.02 (t, J=6.7 Hz, 2H), 3.74 (t, J=10.7 Hz, 1H), 3.00 (d, J=6.2 Hz, 2H), 2.83-2.60 (m, 3H), 1.82-1.62 (m, 2H), 1.60-1.46 (m, 2H), 1.38 (dd, J=14.2, 6.3 Hz, 2H), 1.36-1.15 (m, 14H). 0.89 (dt, J=13.8, 6.9 Hz, 6H).

### Example 31

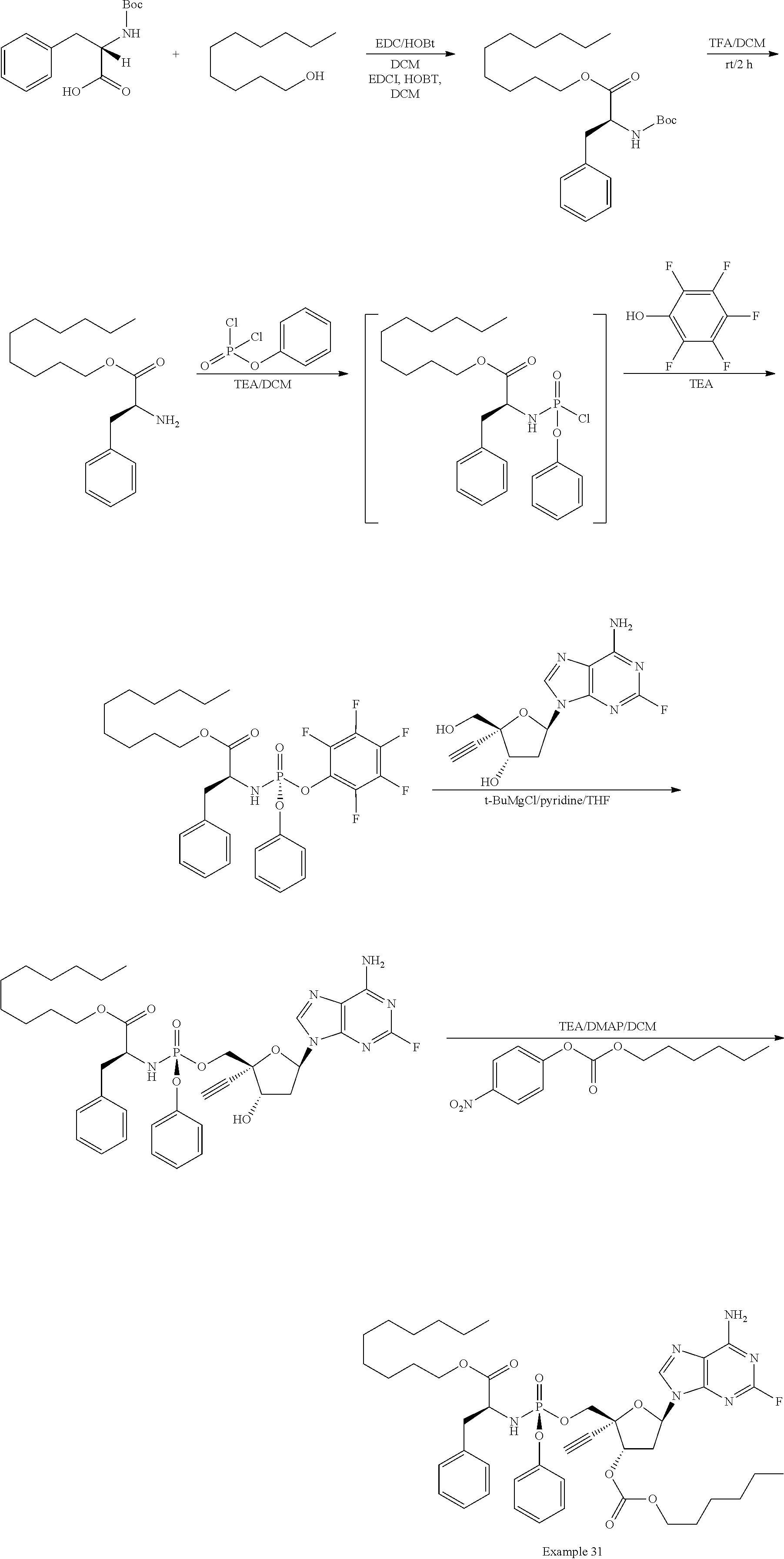

### Step 1: Decyl ((S)-(perfluorophenoxy)(phenoxy)phosphoryl)-L-phenylalaninate

To a stirred solution of decyl L-phenylalaninate (3 g, 9.82 mmol) in DCM (20 mL) was added triethylamine (1.502 mL, 10.80 mmol) dropwise at −70° C. To this mixture was added dropwise a solution of phenyl phosphorodichloridate (2.051 g, 9.72 mmol) in anhydrous DCM (8 mL). The reaction mixture was stirred at this temperature for 30 min and then allowed to warm to 0° C. over 2 h and stirred for 1 h. To this mixture was added dropwise a solution of 2,3,4,5,6-pentafluorophenol (1.790 g, 9.72 mmol) and triethylamine (1.502 mL, 10.80 mmol) in DCM (30 mL) and stirred at 0° C. for 2 h. LCMS showed the reaction was completed. The white solid (triethylamine hydrochloride) was filtered off and washed with DCM (10 mL). The filtrate was concentrated under reduced pressure, the residue was triturated with tert-butylmethyl ether (20 mL), and the triethylamine hydrochloride salt was removed by filtration. The cake was washed with tert-butylmethyl ether (2×10 mL), and the combined filtrate was concentrated under reduced pressure. The resulting residue was triturated with 10% EtOAc/hexanes (30 mL) and solids collected by filtration to give decyl ((S)-(perfluorophenoxy)(phenoxy)phosphoryl)-L-phenylalaninate (1.5 g, 2.390 mmol, 24.34% yield) as white solid (>98% de as determined by ³¹PNMR). LCMS (M+H)=628.0; Retention time (0.1% TFA)=2.78 min. ³¹PNMR (162 MHz, DMSO-d₆) δ 0.22.

### Step 2: Decyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.2 g, 0.682 mmol) in THF (10 mL) and pyridine (2 mL) was added tert-butylmagnesium chloride (1.432 mL, 1.432 mmol) dropwise at 0° C. and stirred at 0° C. for 30 min. A solution of decyl ((S)-(perfluorophenoxy)(phenoxy)phosphoryl)-L-phenylalaninate (0.428 g, 0.682 mmol) in THF (25 mL) was added dropwise to the above solution at −15° C. and stirred at −15° C. for 16 h. TLC showed the presence of new compound. The reaction was quenched with 2M HCl (10 mL) and extracted with EtOAc (50 mL×3). The combined organic phases were washed with a solution of NaHCO₃ (50 mL), brine (50 mL), dried with Na₂SO₄, filtered and concentrated in vacuum. The crude product was purified by flash chromatography (silica gel, 0-10% MeOH/DCM) to afford decyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (160 mg, 0.217 mmol, 31.8% yield) as white solid. ¹HNMR (400 MHz, CDCl₃) δ 7.88 (s, 1H), 7.28 (d, J=8.0 Hz, 2H), 7.25-7.07 (m, 8H), 6.31 (dd, J=7.4, 4.3 Hz, 1H), 6.06-5.72 (m, 2H), 4.71 (d, J=5.3 Hz, 1H), 4.37-4.12 (m, 3H), 4.04 (t, J=6.8 Hz, 2H), 3.71 (s, 1H), 3.47 (d, J=5.2 Hz, 1H), 3.00 (d, J=6.3 Hz, 2H), 2.81-2.55 (m, 3H), 1.53 (s, 2H), 1.26 (d, J=23.2 Hz, 14H), 0.88 (t, J=6.8 Hz, 3H).

### Step 3: Decyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate

To a stirred mixture of decyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (140 mg, 0.190 mmol) in DCM (10 mL) was added triethylamine (0.079 mL, 0.570 mmol) followed by hexyl (4-nitrophenyl) carbonate (102 mg, 0.380 mmol), and the mixture was stirred at room temperature for 2 days. TLC showed the presence of new compound. The reaction mixture was concentrated and purified by flash chromatography (silica gel, 0-10% MeOH/DCM) to give decyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate (130 mg, 0.150 mmol, 79% yield) as white foam. LCMS (M+H)=865.7; Retention time (0.1% TFA)=2.60 min. ¹HNMR (400 MHz, CDCl₃) δ 7.94 (s, 1H), 7.26-7.07 (m, 10H), 6.38 (t, J=6.7 Hz, 1H), 6.05-5.64 (m, 2H), 5.48 (dd, J=6.9, 4.4 Hz, 1H), 4.31 (ddd, J=13.6, 10.4, 5.0 Hz, 2H), 4.17 (dt, J=16.5, 5.8 Hz, 3H), 4.01 (t, J=6.8 Hz, 2H), 3.63 (t, J=10.7 Hz, 1H), 3.00 (d, J=6.3 Hz, 2H), 2.83-2.62 (m, 3H), 1.76-1.69 (m, 2H), 1.57-1.47 (m, 2H), 1.38 (dd, J=15.0, 6.4 Hz, 2H), 1.35-1.21 (m, 18H), 0.89 (dt, J=13.8, 6.9 Hz, 6H).

### Example 32

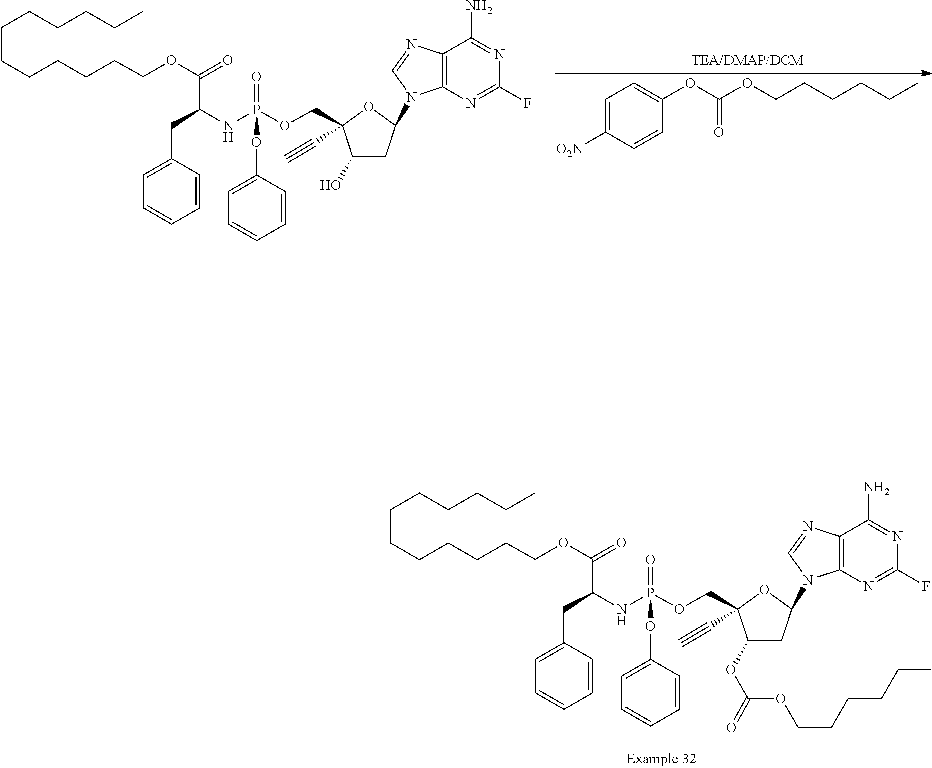 [SMILES: C#C[C@]1(CO[P@@](=O)(N[C@@H](Cc2ccccc2)C(=O)OCCCCCCCCCCCC)Oc2ccccc2)O[C@@H](n2cnc3c(N)nc(F)nc32)C[C@@H]1O ; CCCCCCOC(=O)Oc1ccc([N+](=O)[O-])cc1 ; C#C[C@]1(CO[P@@](=O)(N[C@@H](Cc2ccccc2)C(=O)OCCCCCCCCCCCC)Oc2ccccc2)O[C@@H](n2cnc3c(N)nc(F)nc32)C[C@@H]1OC(=O)OCCCCCC]

### Dodecyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate

To a stirred mixture of dodecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (252 mg, 0.329 mmol) in DCM (10 mL) was added triethylamine (0.138 mL, 0.988 mmol) and DMAP (40.3 mg, 0.329 mmol) followed by hexyl (4-nitrophenyl) carbonate (176 mg, 0.659 mmol) and stirred at room temperature for 2 days. LCMS showed the reaction was completed. The reaction mixture was concentrated and purified by silica gel chromatography (0-5% MeOH/DCM) to give dodecyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate (120 mg, 0.131 mmol, 39.8% yield) as white solid. LCMS (M+H)=892.7; Retention time (0.1% TFA)=2.517 min. ¹HNMR (400 MHz, CDCl₃) δ 8.01 (s, 1H), 7.26-7.04 (m, 10H), 6.37 (d, J=6.4 Hz, 1H), 6.17 (s, 2H), 5.47 (s, 1H), 4.31 (d, J=7.8 Hz, 2H), 4.18 (d, J=6.1 Hz, 3H), 4.01 (d, J=6.4 Hz, 2H), 3.73 (s, 1H), 3.00 (s, 2H), 2.69 (d, J=18.1 Hz, 3H), 1.69 (d, J=6.9 Hz, 2H), 1.51 (s, 2H), 1.46-1.07 (m, 24H), 0.89 (d, J=8.0 Hz, 6H).

### Example 33

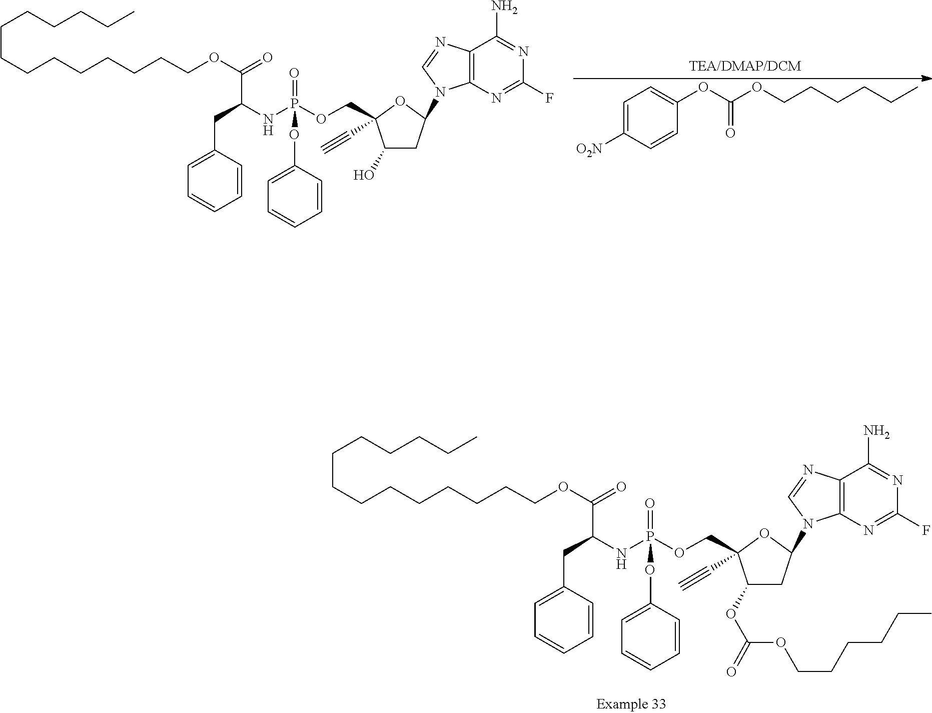 [SMILES: C#C[C@]1(CO[P@@](=O)(N[C@@H](Cc2ccccc2)C(=O)OCCCCCCCCCCCCCC)Oc2ccccc2)O[C@@H](n2cnc3c(N)nc(F)nc32)C[C@@H]1O ; CCCCCCOC(=O)Oc1ccc([N+](=O)[O-])cc1 ; C#C[C@]1(CO[P@@](=O)(N[C@@H](Cc2ccccc2)C(=O)OCCCCCCCCCCCCCC)Oc2ccccc2)O[C@@H](n2cnc3c(N)nc(F)nc32)C[C@@H]1OC(=O)OCCCCCC]

### Tetradecyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate

To a stirred mixture of tetradecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.189 mmol) in DCM (10 mL) was added triethylamine (0.026 mL, 0.189 mmol) and DMAP (23.11 mg, 0.189 mmol) followed by hexyl (4-nitrophenyl) carbonate (101 mg, 0.378 mmol and the reaction mixture was stirred at 23° C. for 2 days. The reaction mixture was concentrated, and the residue was purified by flash chromatography (silica column 40 g, 0-10% MeOH/DCM) to give tetradecyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate (125 mg, 0.135 mmol, 71.4% yield) as white solid. LCMS (M+H)=920.7; Retention time (0.1% TFA)=2.849 min. ¹HNMR (400 MHz, CDCl₃) δ 8.04 (s, 1H), 7.26-7.07 (m, 10H), 6.38 (t, J=6.7 Hz, 1H), 6.16 (s, 2H), 5.47 (dd, J=6.6, 4.5 Hz, 1H), 4.38-4.24 (m, 2H), 4.24-4.09 (m, 3H), 4.02 (t, J=6.7 Hz, 2H), 3.75 (t, J=10.7 Hz, 1H), 3.13-2.89 (m, 2H), 2.81-2.61 (m, 3H), 1.82-1.62 (m, 2H), 1.61-1.46 (m, 2H), 1.46-1.35 (m, 2H), 1.33-1.18 (m, 26H), 0.89 (dt, J=10.1, 6.9 Hz, 6H).

### Example 32

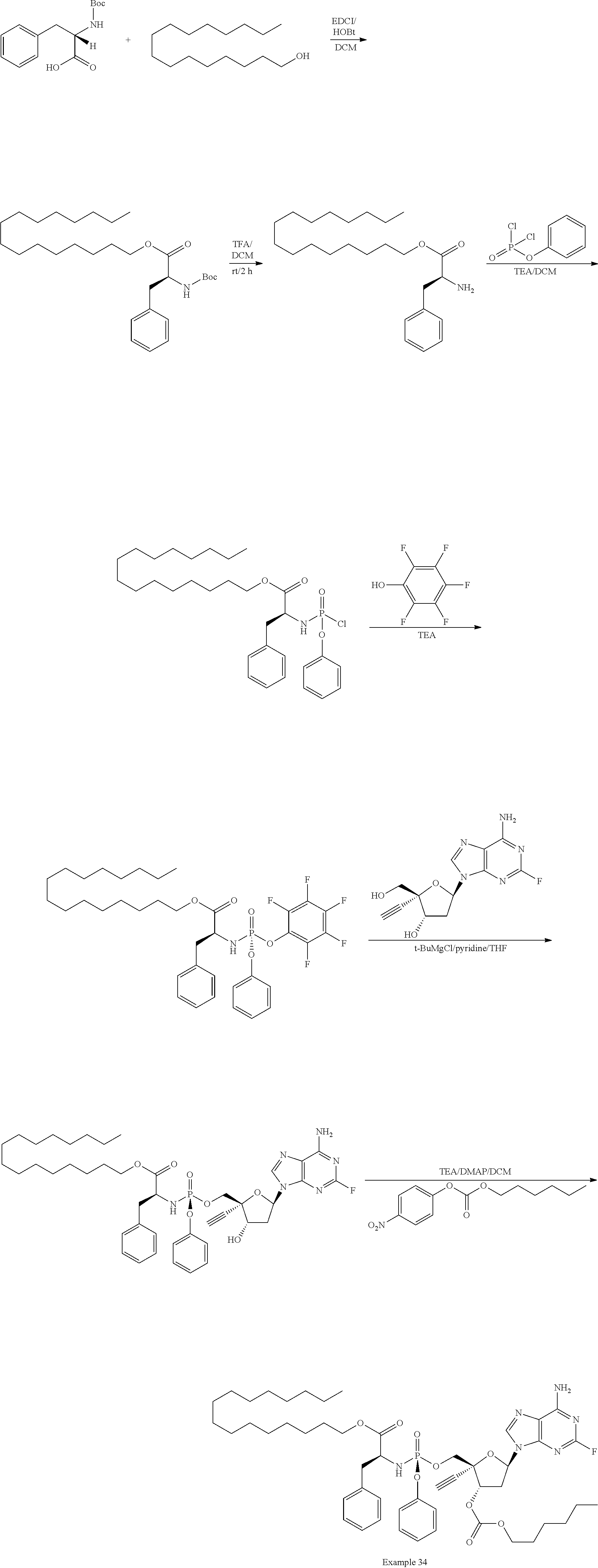

### Step 1: Hexadecyl (tert-butoxycarbonyl)-L-phenylalaninate

To a stirred mixture of (tert-butoxycarbonyl)-L-phenylalanine (20 g, 75 mmol), HOBt (15.28 g, 113 mmol) and EDC (17.34 g, 90 mmol) in DCM (150 mL) was added TEA (10.51 mL, 75 mmol) at 0° C. After 30 min, hexadecan-1-ol (18.28 g, 75 mmol) was added and the resulting reaction mixture was stirred at 25° C. for 16 h. TLC showed the reaction was completed. The reaction mixture was diluted with water (80 mL), organic layer was separated and the aqueous layer was extracted with DCM (100 mL×2). The combined organic phases were washed with brine, dried over Na₂SO₄ and concentrated. The residue was purified by silica gel chromatography (pet. ether:EtOAc=100:010:1) to afford hexadecyl (tert-butoxycarbonyl)-L-phenylalaninate (17.2 g, 35.1 mmol, 46.6% yield) as white solid. ¹HNMR (400 MHz, CDCl₃) δ 7.31-7.27 (m, 2H), 7.26-7.07 (m, 3H), 4.98 (d, J=8.1 Hz, 1H), 4.57 (d, J=7.0 Hz, 1H), 4.08 (dd, J=6.9, 5.7 Hz, 2H), 3.19-2.85 (m, 2H), 1.59 (s, 2H), 1.42 (s, 9H), 1.25 (d, J=6.6 Hz, 22H), 0.88 (t, J=6.8 Hz, 3H).

### Step 2: Hexadecyl L-phenylalaninate

To a stirred solution of hexadecyl (tert-butoxycarbonyl)-L-phenylalaninate (17 g, 34.7 mmol) in DCM (75 mL) was added TFA (30 mL, 389 mmol) at 0° C. After 5 min, the mixture was stirred at 15° C. for 2 h. TLC showed the presence of new compound. The reaction mixture was concentrated and the residue was diluted with DCM (100 mL) and the pH was adjusted to 8-9 with sat.Na₂CO₃. The organic layer was separated and the aqueous layer was extracted with DCM (2×100 mL). The combined organic layers were washed with brine (100 mL), dried over Na₂SO₄, filtered and concentrated. The residue was purified by silica gel chromatography (DCM:MeOH=20:1) to give hexadecyl L-phenylalaninate (13 g, 33.4 mmol, 86% yield). ¹HNMR (400 MHz, CDCl₃) δ 7.30 (t, J=7.2 Hz, 2H), 7.26-7.17 (m, 3H), 4.09 (t, J=6.7 Hz, 2H), 3.72 (dd, J=7.8, 5.4 Hz, 1H), 3.08 (dd, J=13.5, 5.4 Hz, 1H), 2.87 (dd, J=13.5, 7.8 Hz, 1H), 1.59 (d, J=6.8 Hz, 2H), 1.27 (d, J=9.3 Hz, 28H), 0.88 (t, J=6.8 Hz, 3H).

### Step 3: Hexadecyl ((S)-(perfluorophenoxy)(phenoxy)phosphoryl)-L-phenylalaninate

To a stirred solution of hexadecyl L-phenylalaninate (10 g, 25.7 mmol) in anhydrous DCM (100 mL) was added dropwise triethylamine (3.94 mL, 28.2 mmol) at −70° C. over 15 min. To this mixture was added a solution of phenylphosphonic dichloride (5.00 g, 25.7 mmol) in anhydrous DCM (50 mL) over 1 h. The reaction mixture was stirred at this temperature for additional 30 min and then allowed to warm to 0° C. over 2 h and stirred for 1 h. To this mixture was added a solution of 2,3,4,5,6-pentafluorophenol (4.72 g, 25.7 mmol) and TEA (3.94 mL, 28.2 mmol) in DCM (30 mL) over 20 min and stirred at 0° C. for 16 h. TLC showed the reaction was completed. The white solid (triethylamine hydrochloride) was filtered off and washed with DCM (50 mL). The filtrate was concentrated under reduced pressure, the residue was triturated with tert-butylmethyl ether (150 mL), and the triethylamine hydrochloride salt was removed by filtration. The cake was washed with tert-butylmethyl ether (2×100 mL), and the combined filtrate was concentrated under reduced pressure to give 12 g of crude solid containing an even mixture of diastereomers. The mixture was triturated with 20% EtOAc/hexanes (100 mL) and solids collected by filtration to give hexadecyl ((S)-(perfluorophenoxy)(phenoxy)phosphoryl)-L-phenylalaninate (5.2 g, 6.94 mmol, 27.0% yield) as white solid (>98% de as determined by ³¹PNMR). ¹HNMR (400 MHz, CDCl₃) δ 7.35 (t, J=7.8 Hz, 2H), 7.26-7.13 (m, 8H), 7.13-6.93 (m, 2H), 4.44 (ddd, J=15.6, 10.1, 5.8 Hz, 1H), 4.07 (ddt, J=10.7, 6.7, 5.6 Hz, 2H), 3.79 (t, J=11.1 Hz, 1H), 3.19-2.96 (m, 3H), 1.70 (d, J=11.5 Hz, 2H), 1.40 (dd, J=15.5, 8.2 Hz, 1H), 1.35-1.20 (m, 26H), 0.91-0.82 (m, 3H). ³¹PNMR (162 MHz, CDCl₃) δ−1.57.

### Step 4: Hexadecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.3 g, 1.023 mmol) in THF (20 mL) and pyridine (1.0 mL) was added tert-butylmagnesium chloride (2.148 mL, 2.148 mmol) dropwise at 0° C. Then, the reaction was stirred at 25° C. for 30 min. A solution of (2R,3S,5R)-5-(6-amino-2-fluoro-9H-purin-9-yl)-2-ethynyl-2-(hydroxymethyl)tetrahydrofuran-3-ol (0.3 g, 1.023 mmol) in THF (20 mL) was added dropwise to the above solution at −15° C. and stirred at −15° C. for 12 h. LCMS showed the reaction was completed. The reaction was quenched with 2M HCl (10 mL) and extracted with EtOAc (20 mL×3). The combined organic layers were washed with a solution of NaHCO₃ (20 mL), brine (20 mL), dried over Na₂SO₄, filtered and concentrated in vacuum. The crude product was purified by the silica gel chromatography (25 g, DCM:MeOH=20:1) to give hexadecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (301 mg, 0.348 mmol, 34.0% yield). ¹HNMR (400 MHz, CDCl₃) δ 7.90 (s, 1H), 7.25-7.01 (m, 10H), 6.31 (dd, J=7.3, 4.3 Hz, 1H), 6.02 (s, 1H), 4.72 (t, J=7.4 Hz, 1H), 4.23 (dt, J=20.5, 8.5 Hz, 3H), 4.03 (t, J=6.5 Hz, 2H), 3.00 (d, J=5.9 Hz, 2H), 2.83-2.53 (m, 2H), 1.56 (d, J=27.4 Hz, 3H), 1.35-1.14 (m, 28H), 0.88 (t, J=6.7 Hz, 3H).

### Step 5: Hexadecyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate

To a stirred mixture of hexadecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (300 mg, 0.365 mmol) in DCM (20 mL) was added triethylamine (0.153 mL, 1.096 mmol), DMAP (44.6 mg, 0.365 mmol) and hexyl (4-nitrophenyl) carbonate (195 mg, 0.731 mmol) under N₂ at 15° C. and stirred at 20° C. for 2 days. LCMS showed the presence of new product. The reaction mixture was concentrated and the residue was purified by flash chromatography (silica gel, 0-10% MeOH/DCM) to give hexadecyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate (120 mg, 0.124 mmol, 34.0% yield). LCMS (M+H)=948.7; Retention time (0.1% TFA)=3.80 min. ¹HNMR (400 MHz, CDCl₃) δ 7.95 (s, 1H), 7.27-7.03 (m, 10H), 6.48-6.26 (m, 1H), 6.01 (s, 1H), 5.48 (dd, J=6.8, 4.4 Hz, 1H), 4.46-4.23 (m, 2H), 4.25-4.11 (m, 3H), 4.01 (t, J=6.7 Hz, 2H), 3.70 (t, J=10.7 Hz, 1H), 3.02 (t, J=10.6 Hz, 2H), 2.80-2.61 (m, 3H), 1.75-1.63 (m, 2H), 1.36-1.17 (m, 34H), 0.89 (dt, J=10.3, 6.9 Hz, 6H).

### Example 35

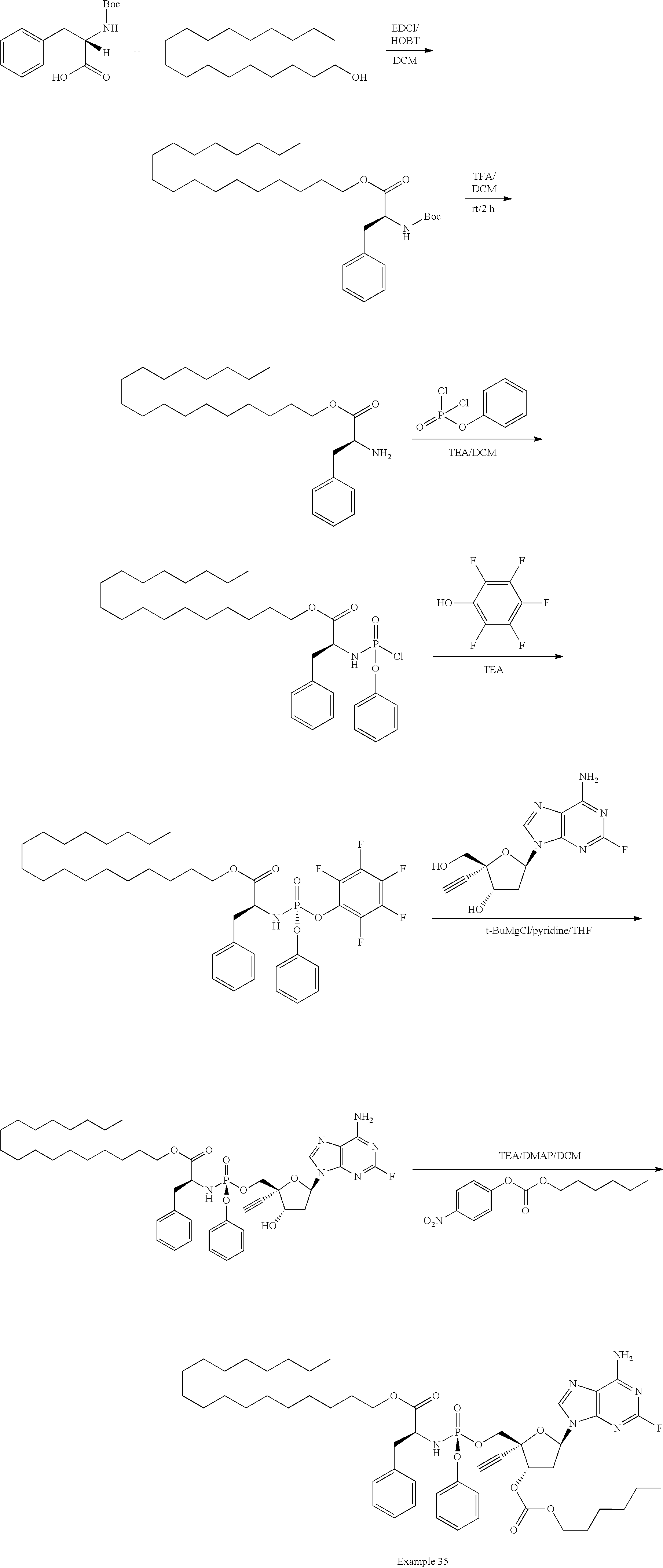

### Step 1: Octadecyl (tert-butoxycarbonyl)-L-phenylalaninate

To a stirred mixture of (tert-butoxycarbonyl)-L-phenylalanine (20 g, 75 mmol), HOBt (15.28 g, 113 mmol) and EDC (17.34 g, 90 mmol) in DCM (150 mL) was added TEA (10.51 mL, 75 mmol) at 0° C. After 30 min, octadecan-1-ol (20.39 g, 75 mmol) was added and the resulting reaction mixture was stirred at 25° C. for 16 h. TLC showed the reaction was completed. The reaction mixture was diluted with water (40 mL), the organic layer was separated and the aqueous layer was extracted with DCM (50 mL×2). The combined organic layers were washed with brine, dried over Na₂SO₄ and concentrated. The residue was purified by silica gel chromatography (pet. ether:EtOAc=100:010:1) to give octadecyl (tert-butoxycarbonyl)-L-phenylalaninate (20.2 g, 39.0 mmol, 51.8% yield) a yellow oil. ¹HNMR (400 MHz, CDCl₃) δ 7.32-7.27 (m, 2H), 7.26-7.11 (m, 3H), 4.98 (d, J=8.1 Hz, 1H), 4.57 (d, J=7.0 Hz, 1H), 4.12-4.04 (m, 2H), 3.14-3.00 (m, 2H), 1.65-1.53 (m, 4H), 1.42 (s, 9H), 1.29-1.22 (m, 28H), 0.88 (t, J=6.8 Hz, 3H).

### Step 2: Octadecyl L-phenylalaninate

To a stirred cold (ice-water bath) solution of octadecyl (tert-butoxycarbonyl)-L-phenylalaninate (20 g, 38.6 mmol) in DCM (150 mL) was added TFA (50 mL, 649 mmol). After 5 min, the mixture was stirred at room temperature for 2 h. TLC showed the presence of new compound. The reaction mixture was concentrated, the residue was diluted with DCM (100 mL) and the pH was adjusted to 8-9 with sat.Na₂CO₃. The organic layer was separated and the aqueous layer was extracted with DCM (2×100 mL). The combined organic layers were washed with brine (80 mL), dried over Na₂SO₄, filtered and concentrated. The residue was purified by silica gel chromatography (DCM:MeOH=20:1) to give octadecyl L-phenylalaninate (14.6 g, 35.0 mmol, 53.9% yield). ¹HNMR (400 MHz, CDCl₃) δ 7.30 (t, J=7.2 Hz, 2H), 7.26-7.17 (m, 3H), 4.09 (t, J=6.7 Hz, 2H), 3.72 (dd, J=7.8, 5.4 Hz, 1H), 3.08 (dd, J=13.5, 5.4 Hz, 1H), 2.87 (dd, J=13.5, 7.8 Hz, 1H), 1.59 (d, J=6.8 Hz, 2H), 1.27 (d, J=9.3 Hz, 30H), 0.88 (t, J=6.8 Hz, 3H).

### Step 3: Octadecyl ((S)-(perfluorophenoxy)(phenoxy)phosphoryl)-L-phenylalaninate

To a stirred solution of octadecyl L-phenylalaninate (14.1 g, 33.8 mmol) in anhydrous DCM (200 mL) was added dropwise triethylamine (5.16 mL, 37.1 mmol) at −70° C. over 15 min. To this mixture was added a solution of phenyl phosphorodichloridate (7.05 g, 33.4 mmol) in anhydrous DCM (50 mL) over 1 h. The reaction mixture was stirred at this temperature for additional 30 min and then allowed to warm to 0° C. over 2 h and stirred for 1 h. To this mixture was added a solution of 2,3,4,5,6-pentafluorophenol (6.15 g, 33.4 mmol) and triethylamine (5.16 mL, 37.1 mmol) in DCM (50 mL) over 20 min and stirred at 0° C. for 4 h. TLC showed the reaction was completed. The white solid (triethylamine hydrochloride) was filtered off and washed with DCM (50 mL). The filtrate was concentrated under reduced pressure, and then the residue was triturated with tert-butylmethyl ether (500 mL). The triethylamine hydrochloride salt was removed by filtration. The cake was washed with tert-butylmethyl ether (2×50 mL), and the combined filtrate was concentrated under reduced pressure to give 25 g of crude solid containing an even mixture of diastereomers. The mixture was triturated with 20% EtOAc/hexanes (400 mL) and solids collected by filtration to give octadecyl ((S)-(perfluorophenoxy)(phenoxy)phosphoryl)-L-phenylalaninate (10 g, 13.52 mmol, 40.0% yield) as white solid (>98% de as determined by ³¹PNMR: N76107-96-A1). ¹HNMR (400 MHz, CDCl₃) δ 7.35 (t, J=7.8 Hz, 2H), 7.26-7.13 (m, 6H), 7.05 (d, J=7.4 Hz, 2H), 4.44 (s, 1H), 4.13-3.95 (m, 2H), 3.77 (t, J=11.1 Hz, 1H), 3.10 (ddd, J=39.1, 13.8, 6.0 Hz, 2H), 1.54 (s, 2H), 1.26 (s, 30H), 0.88 (t, J=6.7 Hz, 3H). ³¹PNMR (162 MHz, CDCl₃) δ−1.57.

### Step 4: Octadecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.3 g, 1.023 mmol) in THF (40 mL) and pyridine (2.000 mL) was added dropwise tert-butylmagnesium chloride (2.148 mL, 2.148 mmol) at 0° C. and the reaction was stirred at 25° C. for 30 min. A solution of octadecyl ((S)-(perfluorophenoxy)(phenoxy)phosphoryl)-L-phenylalaninate (0.984 g, 1.330 mmol) in THF (20 mL) was added dropwise to the above solution at −15° C. and stirred at −15° C. for 4 h. TLC showed the presence of new compound. The reaction mixture was quenched with 2M HCl (20 mL) and extracted with EtOAc (30 mL×3). The combined organic layers were washed with a solution of NaHCO₃ (20 mL), brine (20 mL), dried with Na₂SO₄, filtered and concentrated in vacuum. The crude product was purified by silica gel chromatography (0-10% MeOH/DCM) to give octadecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (320 mg, 0.362 mmol, 35.4% yield) as white solid. ¹HNMR (400 MHz, CDCl₃) δ 7.90 (s, 1H), 7.26-7.04 (m, 10H), 6.31 (dd, J=7.1, 4.4 Hz, 1H), 6.28-5.93 (m, 2H), 4.71 (s, 1H), 4.35-4.15 (m, 3H), 4.02 (t, J=6.7 Hz, 2H), 3.95 (t, J=11.0 Hz, 1H), 3.82 (s, 1H), 2.99 (d, J=6.2 Hz, 2H), 2.81-2.61 (m, 3H), 1.51 (s, 2H), 1.24 (d, J=9.7 Hz, 30H), 0.88 (t, J=6.8 Hz, 3H).

### Step 5: Octadecyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate

To a stirred mixture of octadecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (320 mg, 0.377 mmol) in DCM (20 mL) was added triethylamine (0.158 mL, 1.131 mmol), DMAP (46.0 mg, 0.377 mmol) and hexyl (4-nitrophenyl) carbonate (403 mg, 1.508 mmol) under N₂ at 15° C. The mixture was stirred at room temperature for 3 days. LCMS showed the reaction was completed. The reaction mixture was concentrated in vacuum and the residue was purified by silica gel chromatography (25 g, 0-7% MeOH/DCM) to give octadecyl ((S)-(((2R,3S,5R)-5-(6-amino-2-fluoro-9H-purin-9-yl)-2-ethynyl-3-(((hexyloxy)carbonyl)oxy)tetrahydrofu-ran-2-yl)methoxy)(phenoxy)phosphoryl)-L-phenylalaninate (191 mg, 0.188 mmol, 49.8% yield) as pale yellow solid. LCMS: Retention time (0.1% TFA)=4.08 min. ¹HNMR (400 MHz, CDCl₃) δ 7.95 (s, 1H), 7.26-7.00 (m, 10H), 6.92-6.44 (m, 2H), 6.37 (s, 1H), 5.48 (d, J=2.5 Hz, 1H), 4.30 (dd, J=11.2, 6.5 Hz, 2H), 4.24-4.07 (m, 4H), 4.01 (t, J=6.4 Hz, 2H), 3.11-2.90 (m, 2H), 2.67 (s, 3H), 1.78-1.65 (m, 2H), 1.51 (s, 2H), 1.44-1.15 (m, 36H), 0.89 (dt, J=10.3, 6.9 Hz, 6H).

### Example 36

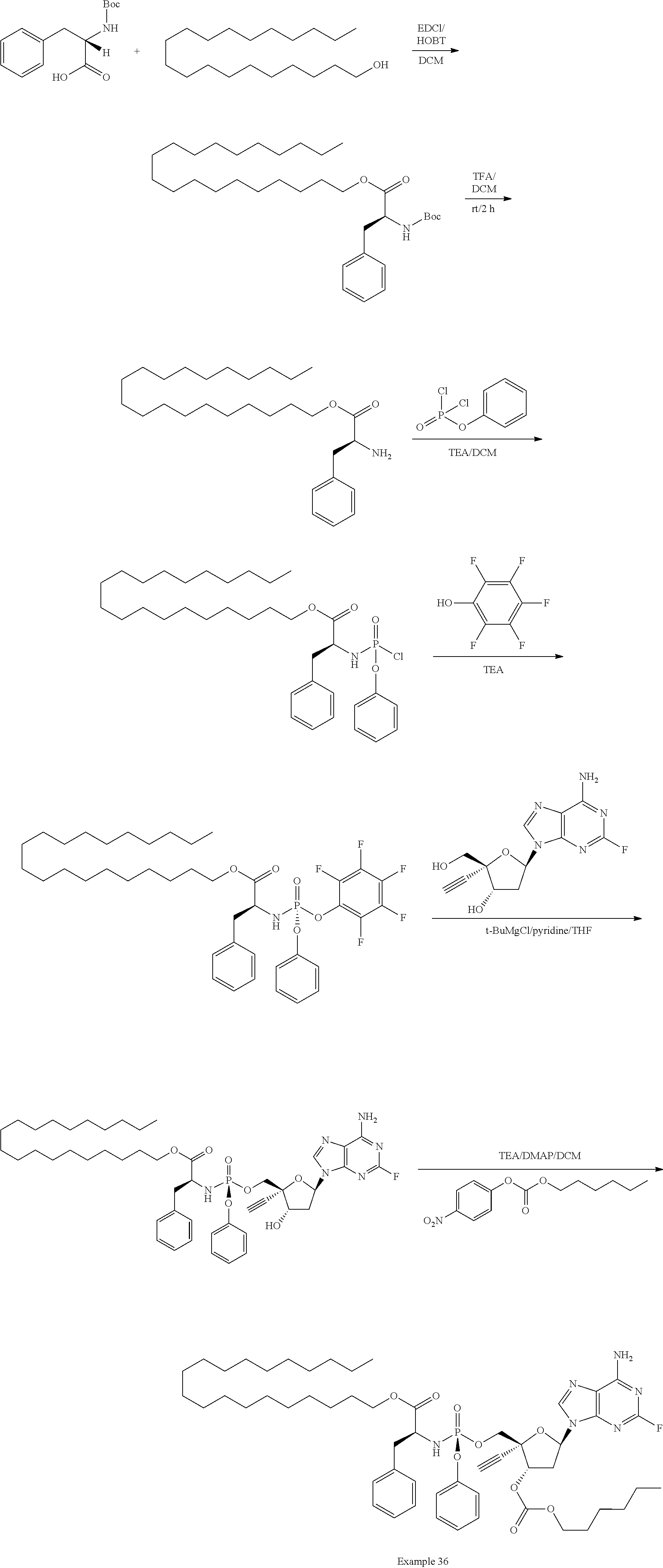

### Step 1: Icosyl (tert-butoxycarbonyl)-L-phenylalaninate

To a stirred mixture of (tert-butoxycarbonyl)-L-phenylalanine (20 g, 75 mmol), HOBt (15.28 g, 113 mmol) and EDC (17.34 g, 90 mmol) in DCM (150 mL) was added TEA (10.51 mL, 75 mmol) at 0° C. After 30 min, icosan-1-ol (22.51 g, 75 mmol) was added and the resulting reaction mixture was stirred at 25° C. for 16 h. TLC showed the reaction was completed. The reaction mixture was diluted with water (80 mL), the organic layer was separated and the aqueous layer was extracted with DCM (100 mL×2). The combined organic layers were washed with brine, dried over Na₂SO₄ and concentrated. The residue was purified by silica gel chromatography (pet. ether:EtOAc=100:010:1) to afford icosyl (tert-butoxycarbonyl)-L-phenylalaninate (20 g, 36.6 mmol, 48.6% yield) as white solid. ¹HNMR (400 MHz, CDCl₃) δ 7.33-7.27 (m, 2H), 7.25-7.08 (m, 3H), 4.98 (d, J=8.2 Hz, 1H), 4.57 (d, J=7.5 Hz, 1H), 4.07 (tt, J=5.4, 2.7 Hz, 2H), 3.09 (dd, J=13.9, 7.3 Hz, 2H), 1.57-1.52 (m, 2H), 1.42 (s, 9H), 1.26 (s, 34H), 0.88 (t, J=6.8 Hz, 3H).

### Step 2: Icosyl L-phenylalaninate

To a stirred cold (ice-water bath) solution of icosyl (tert-butoxycarbonyl)-L-phenylalaninate (19.5 g, 35.7 mmol) in DCM (150 mL) was added 2,2,2-trifluoroacetic acid. After 5 min, the mixture was stirred at 25° C. for 2 h. TLC showed presence of new compound. The reaction mixture was concentrated and the residue was diluted with DCM (100 mL) and the pH was adjusted to 8-9 with sat.Na₂CO₃. the organic layer was separated and the aqueous layer was extracted with DCM (2×100 mL). The combined organic layers were washed with brine (80 mL), dried over Na₂SO₄, filtered and concentrated. The residue was purified by silica gel chromatography (DCM:MeOH=20:1) to give icosyl L-phenylalaninate (14.5 g, 32.5 mmol, 91% yield). ¹HNMR (400 MHz, CDCl₃) δ 7.30 (t, J=7.2 Hz, 2H), 7.26-7.17 (m, 3H), 4.09 (t, J=6.7 Hz, 2H), 3.72 (dd, J=7.8, 5.4 Hz, 1H), 3.08 (dd, J=13.5, 5.4 Hz, 1H), 2.87 (dd, J=13.5, 7.8 Hz, 1H), 1.59 (d, J=6.8 Hz, 2H), 1.27 (d, J=9.3 Hz, 34H), 0.88 (t, J=6.8 Hz, 3H).

### Step 3: Icosyl ((S)-(perfluorophenoxy)(phenoxy)phosphoryl)-L-phenylalaninate

To a stirred solution of icosyl L-phenylalaninate (12.6 g, 28.3 mmol) in anhydrous DCM (100 mL) was added dropwise triethylamine (4.32 mL, 31.1 mmol) at −70° C. over 15 min. To this mixture was added a solution of phenyl phosphorodichloridate (5.90 g, 28.0 mmol) in anhydrous DCM (35 mL) over 1 h. The reaction mixture was stirred at this temperature for additional 30 min and then allowed to warm to 0° C. over 2 h and stirred for 1 h. To this mixture was added a solution of 2,3,4,5,6-pentafluorophenol (5.15 g, 28.0 mmol) and triethylamine (4.32 mL, 31.1 mmol) in DCM (30 mL) over 20 min and stirred at 0° C. for 4 h. TLC showed the reaction was completed. The white solid (triethylamine hydrochloride) was filtered off and washed with DCM (50 mL). The filtrate was concentrated under reduced pressure, the residue was triturated with tert-butylmethyl ether (250 mL), and the triethylamine hydrochloride salt was removed by filtration. The cake was washed with tert-butylmethyl ether (2×50 mL), and the combined filtrate was concentrated under reduced pressure to give 26 g crude solid containing an even mixture of diastereomers. The mixture was triturated with 20% EtOAc/hexanes (200 mL) and solids collected by filtration to give icosyl ((S)-(perfluorophenoxy)(phenoxy)phosphoryl)-L-phenylalaninate (5 g, 6.51 mmol, 23.04% yield) as white solid (>98% de as determined by ³¹PNMR). ¹HNMR (400 MHz, CDCl₃) δ 7.35 (t, J=7.8 Hz, 2H), 7.26-7.13 (m, 6H), 7.05 (d, J=7.4 Hz, 2H), 4.44 (s, 1H), 4.13-3.95 (m, 2H), 3.77 (t, J=11.1 Hz, 1H), 3.10 (ddd, J=39.1, 13.8, 6.0 Hz, 2H), 1.54 (s, 2H), 1.26 (s, 34H), 0.88 (t, J=6.7 Hz, 3H). ³¹PNMR (162 MHz, CDCl₃) δ−1.57.

### Step 4: Icosyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.3 g, 1.023 mmol) in THF (40 mL) and pyridine (2.000 mL) was added dropwise tert-butylmagnesium chloride (2.148 mL, 2.148 mmol) at 0° C. Then, the reaction was stirred at 25° C. for 30 min. A solution of icosyl ((S)-(perfluorophenoxy)(phenoxy)phosphoryl)-L-phenylalaninate (1.257 g, 1.637 mmol) in THF (20 mL) was added dropwise to the above solution at −15° C. and stirred at −15° C. for 4 h. TLC showed the presence of new compound. The reaction was quenched with 2M HCl (20 mL) and extracted with EtOAc (50 mL×3). The combined organic layers were washed with a solution of NaHCO₃ (20 mL), brine (20 mL), dried with Na₂SO₄, filtered and concentrated in vacuum to. The crude product was purified by silica gel chromatography (DCM/MeOH=100/0 to 10/1) to give icosyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (330 mg, 0.365 mmol, 35.6% yield) as white solid. ¹HNMR (400 MHz, CDCl₃) δ 7.90 (s, 1H), 7.26-7.04 (m, 10H), 6.31 (dd, J=7.1, 4.4 Hz, 1H), 6.28-5.93 (m, 2H), 4.71 (s, 1H), 4.35-4.15 (m, 3H), 4.02 (t, J=6.7 Hz, 2H), 3.95 (t, J=11.0 Hz, 1H), 3.82 (s, 1H), 2.99 (d, J=6.2 Hz, 2H), 2.81-2.61 (m, 3H), 1.51 (s, 2H), 1.24 (d, J=9.7 Hz, 34H), 0.88 (t, J=6.8 Hz, 3H).

### Step 5: Icosyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate

To a stirred mixture of icosyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (330 mg, 0.376 mmol) in DCM (20 mL) was added triethylamine (0.157 mL, 1.129 mmol), DMAP (46.0 mg, 0.376 mmol) and hexyl (4-nitrophenyl) carbonate (402 mg, 1.505 mmol) under N₂ at 15° C. The mixture was stirred at 15° C. for 3 days. TLC showed the reaction was completed. The reaction mixture was concentrated in vacuum and the residue was purified by silica gel chromatography (25 g, 0-7% MeOH/DCM) to give icosyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate (150 mg, 0.149 mmol, 39.7% yield) as white solid. HPLC: RT (10 mM NH₄HCO₃)=22.56 min. ¹H NMR: N78913-5-A1, ¹HNMR (400 MHz, CDCl₃) δ 7.97 (s, 1H), 7.26-7.04 (m, 10H), 6.38 (s, 1H), 6.32-5.98 (m, 2H), 5.47 (s, 1H), 4.31 (s, 2H), 4.19 (s, 3H), 4.02 (s, 2H), 3.77 (s, 1H), 3.01 (s, 2H), 2.67 (s, 3H), 1.69 (d, J=6.8 Hz, 2H), 1.52 (s, 2H), 1.45-1.16 (m, 40H), 0.95-0.83 (m, 6H).

### Example 37

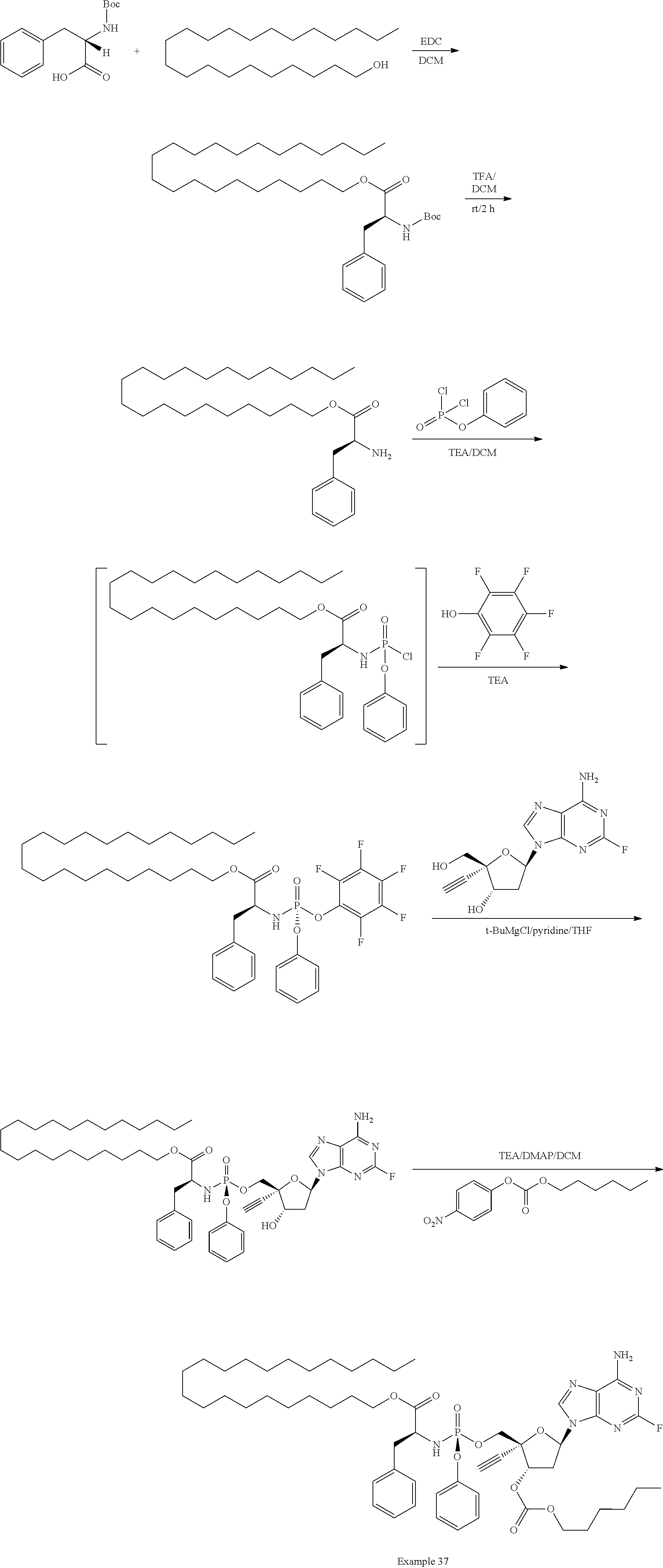

### Step 1: Docosyl (tert-butoxycarbonyl)-L-phenylalaninate

A mixture of docosan-1-ol (19.70 g, 60.3 mmol), (tert-butoxycarbonyl)-L-phenylalanine (16 g, 60.3 mmol), EDC (17.34 g, 90 mmol), imidazole (12.32 g, 181 mmol), DIPEA (31.6 mL, 181 mmol) and (tert-butoxycarbonyl)-L-phenylalanine (16 g, 60.3 mmol) in DCM (500 mL) was stirred at 25° C. for 16 h. TLC showed the presence of new compound. The reaction mixture was diluted with water (500 mL), the organic layer was separated and aqueous layer was extracted with DCM (250 mL×3). The combined organic phases were washed with brine, dried over Na₂SO₄, filtered and concentrated. The residue was purified by silica gel chromatography (pet. ether:EtOAc=20:1) to afford docosyl (tert-butoxycarbonyl)-L-phenylalaninate (12 g, 20.91 mmol, 34.7% yield) as white solid. HNMR: N76106-76-A1. ¹HNMR (400 MHz, CDCl₃) δ 7.33-7.06 (m, 5H), 4.98 (d, J=7.9 Hz, 1H), 4.57 (d, J=7.0 Hz, 1H), 4.17-3.94 (m, 2H), 3.15-2.99 (m, 2H), 1.65-1.56 (m, 2H), 1.42 (s, 9H), 1.26 (s, 38H), 0.88 (t, J=6.8 Hz, 3H).

### Step 2: Docosyl L-phenylalaninate

To a stirred cold (ice-water bath) solution of docosyl (tert-butoxycarbonyl)-L-phenylalaninate (12 g, 20.91 mmol) in DCM (500 mL) was added TFA (16.11 mL, 209 mmol). After 5 min, the mixture was stirred at room temperature for 2 h. TLC showed the presence of new compound. The reaction mixture was washed with water and sat.Na₂CO₃. The combined aqueous layers were extracted with DCM (400 ml×3). The combined organic layers were washed with brine, dried over Na₂SO₄, filtered and concentrated. The residue was purified by silica gel chromatography (DCM:MeOH=20:1) to give docosyl L-phenylalaninate (10 g, 19.00 mmol, 91% yield). ¹HNMR (400 MHz, CDCl₃) δ 7.43-6.96 (m, 5H), 4.09 (t, J=6.7 Hz, 2H), 3.72 (dd, J=7.8, 5.4 Hz, 1H), 3.08 (dd, J=13.5, 5.4 Hz, 1H), 2.87 (dd, J=13.5, 7.8 Hz, 1H), 1.71-1.51 (m, 4H), 1.26 (s, 38H), 0.88 (t, J=6.8 Hz, 3H).

### Step 3: Docosyl ((S)-(perfluorophenoxy)(phenoxy)phosphoryl)-L-phenylalaninate

To a stirred solution of docosyl L-phenylalaninate (16 g, 33.8 mmol) in anhydrous DCM (100 mL) was added dropwise triethylamine (5.16 mL, 37.1 mmol) at −70° C. over 15 min. To this mixture was added a solution of phenyl phosphorodichloridate (7.05 g, 33.4 mmol) in anhydrous DCM (35 mL) over 1 h. The reaction mixture was stirred at this temperature for additional 30 min and then allowed to warm to 0° C. over 2 h and stirred for 1 h. To this mixture was added a solution of 2,3,4,5,6-pentafluorophenol (6.15 g, 33.4 mmol) and triethylamine (5.16 mL, 37.1 mmol) in DCM (30 mL) over 20 min and stirred at 0° C. for 4 h. TLC showed reaction was completed. The white solid (triethylamine hydrochloride) was filtered off and washed with DCM (50 mL). The filtrate was concentrated under reduced pressure, the residue was triturated with tert-butylmethyl ether (250 mL), and the triethylamine hydrochloride salt was removed by filtration. The cake was washed with tert-butylmethyl ether (2×50 mL), and the combined filtrate was concentrated under reduced pressure to give 21 g crude solid containing an even mixture of diastereomers. The mixture was triturated with 20% EtOAc/hexanes (200 mL) and solids collected by filtration to give docosyl ((S)-(perfluorophenoxy)(phenoxy)phosphoryl)-L-phenylalaninate (5.5 g, 6.91 mmol, 20.46% yield) as white solid (>98% de as determined by ³¹PNMR: N76107-98-A1). ¹HNMR (400 MHz, CDCl₃) δ 7.35 (t, J=7.8 Hz, 2H), 7.26-7.13 (m, 6H), 7.05 (d, J=7.4 Hz, 2H), 4.44 (s, 1H), 4.13-3.95 (m, 2H), 3.77 (t, J=11.1 Hz, 1H), 3.10 (ddd, J=39.1, 13.8, 6.0 Hz, 2H), 1.54 (s, 2H), 1.26 (s, 38H), 0.88 (t, J=6.7 Hz, 3H). ³¹PNMR (162 MHz, CDCl₃) δ−1.57.

### Step 4: Docosyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.3 g, 1.023 mmol) in THF (40 mL) and pyridine (2.0 mL) was added dropwise tert-butylmagnesium chloride (2.148 mL, 2.148 mmol) at 0° C. Then, the reaction was stirred at 25° C. for 30 min. A solution of docosyl ((S)-(perfluorophenoxy)(phenoxy)phosphoryl)-L-phenylalaninate (1.628 g, 2.046 mmol) in THF (20 mL) was added dropwise to the above solution at −15° C. and stirred at −15° C. for 4 h. TLC showed presence of new compound. The reaction was quenched with 2M HCl (20 mL) and extracted with EtOAc (30 mL×3). The combined organic layers were washed with a solution of NaHCO₃ (20 mL), brine (20 mL), dried with Na₂SO₄, filtered and concentrated in vacuum to. The crude product was purified by column chromatography (0-10% MeOH/DCM) to give docosyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (340 mg, 0.362 mmol, 35.4% yield) as white solid. ¹HNMR (400 MHz, CDCl₃) δ 7.90 (s, 1H), 7.26-7.06 (m, 10H), 6.31 (dd, J=7.1, 4.4 Hz, 1H), 6.26-5.83 (m, 2H), 4.72 (s, 1H), 4.35-4.17 (m, 3H), 4.07-3.95 (m, 3H), 3.80 (s, 1H), 2.99 (d, J=6.2 Hz, 2H), 2.83-2.55 (m, 3H), 1.51 (s, 2H), 1.36-1.04 (m, 38H), 0.88 (t, J=6.8 Hz, 3H).

### Step 5: Docosyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate

To a stirred mixture of docosyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (340 mg, 0.376 mmol) in DCM (20 mL) was added triethylamine (0.157 mL, 1.127 mmol), DMAP (45.9 mg, 0.376 mmol) and hexyl (4-nitrophenyl) carbonate (402 mg, 1.503 mmol) under N₂ at 15° C. The mixture was stirred at room temperature for 3 days. TLC showed the reaction was completed. The reaction was concentrated in vacuum and the residue was purified by silica gel chromatography (25 g, 0-7% MeOH/DCM) to give docosyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate (166 mg, 0.161 mmol, 42.8% yield) as white solid. HPLC: RT (10 mM NH₄HCO₃)=17.516 min. ¹H NMR: N78913-6-A1, ¹HNMR (400 MHz, CDCl₃) δ 7.95 (s, 1H), 7.27-7.02 (m, 10H), 6.38 (t, J=6.7 Hz, 3H), 5.60-5.40 (m, 1H), 4.30 (dd, J=10.9, 6.4 Hz, 2H), 4.19 (t, J=6.4 Hz, 3H), 4.01 (t, J=6.7 Hz, 3H), 3.08-2.91 (m, 2H), 2.70 (d, J=30.0 Hz, 3H), 1.80-1.66 (m, 2H), 1.51 (s, 2H), 1.43-1.10 (m, 44H), 0.89 (dt, J=10.2, 6.8 Hz, 6H).

### Example 38

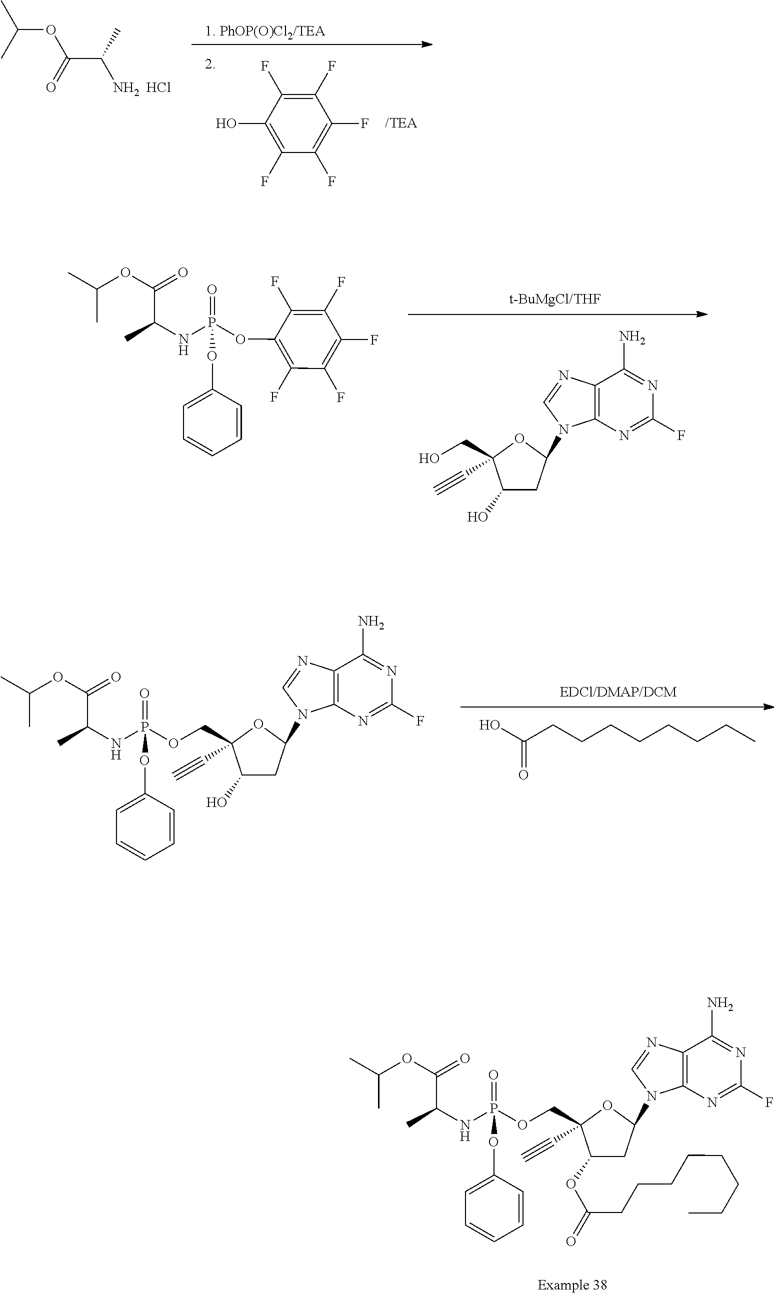

### Step 1: Isopropyl ((S)-(perfluorophenoxy)(phenoxy)phosphoryl)-L-alaninate (N76107-86)

To a stirred solution of isopropyl L-alaninate hydrochloride (8.0 g, 47.7 mmol) in anhydrous dichloromethane (50 mL) was added dropwise TEA (13.77 mL, 99 mmol) at −70° C. over 15 min. To this mixture was added a solution of phenylphosphonic dichloride (9.21 g, 47.2 mmol) in anhydrous dichloromethane (50 mL) over 1 h. The reaction mixture was stirred at this temperature for additional 30 min and then allowed to warm to 0° C. over 2 h and stirred for 1 h. To this mixture was added a solution of 2,3,4,5,6-pentafluorophenol (8.70 g, 47.2 mmol) and TEA (7.32 mL, 52.5 mmol) in dichloromethane (30 mL) over 20 min. The crude mixture was allowed to stir at 0° C. for 16 h. LCMS showed the reaction was completed. The white solid (triethylamine hydrochloride) was filtered off and washed with dichloromethane (25 mL). The filtrate was concentrated under reduced pressure, the residue was triturated with TBME (150 mL), and the triethylamine hydrochloride salt was removed by filtration. The cake was washed with TBME (2×25 mL), and the combined filtrate was concentrated under reduced pressure to give 22 g of crude solid containing an even mixture of diastereomers. The mixture was triturated with 20% EtOAc in hexanes (100 mL) and collected by filtration to give isopropyl ((S)-(perfluorophenoxy)(phenoxy)phosphoryl)-L-alaninate (7.0 g, 15.44 mmol, 32.4% yield) as a white solid (>98% de as determined by ³¹PNMR). ¹H NMR (400 MHz, CDCl₃) δ 7.36 (t, J=7.9 Hz, 2H), 7.26-7.17 (m, 3H), 5.04 (dt, J=12.5, 6.3 Hz, 1H), 4.14 (dd, J=16.1, 8.3 Hz, 1H), 4.08-3.87 (m, 1H), 1.46 (d, J=7.0 Hz, 3H), 1.25 (t, J=5.9 Hz, 6H). ³¹PNMR (162 MHz, CDCl₃) δ−1.60.

### Step 2: Isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (N78870-36)

To a solution of (2R,3S,5R)-5-(6-amino-2-fluoro-9H-purin-9-yl)-2-ethynyl-2-(hydroxymethyl)tetrahydrofuran-3-ol (0.25 g, 0.852 mmol) in THF (50 mL) and pyridine (2.50 mL) was added tert-butylmagnesium chloride (1.705 mL, 1.705 mmol) dropwise at −15° and stirred at −15° C. for 1 h. Then, to the reaction mixture was added a solution of isopropyl ((S)-(perfluorophenoxy)(phenoxy)phosphoryl)-L-alaninate (0.502 g, 1.108 mmol) in THF (15 mL) and the resulting reaction mixture was stirred at −15° C. for 2 h. LCMS showed product/by-product=1/1. The reaction mixture was quenched with NH₄Cl (4 mL) and partitioned between EtOAc (200 mL) and H₂O (30 mL). The organic layer was washed with brine (20 mL), dried over Na₂SO₄, filtered and concentrated in vacuum. The residue was purified by silica gel column chromatography (8% MeOH in DCM) to give isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (90 mg, 0.154 mmol, 18.08% yield) as a white solid. LCMS (M+H)=563.3; Retention time (0.05% TFA)=1.44 min.

### Step 3: (2R,3S,5R)-5-(6-Amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl nonanoate

To a solution of nonanoic acid (50.6 mg, 0.320 mmol) in DCM (8 mL) was added N,N-dimethylpyridin-4-amine (32.6 mg, 0.267 mmol) and 3-(((ethylimino)methylene)amino)-N,N-dimethylpropan-1-amine hydrochloride (102 mg, 0.533 mmol), and the resulting mixture was stirred for 0.5 h at an ice-water bath temperature. Then, isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.267 mmol) was added, and the resulting mixture was stirred at 25° C. for 16 h. LCMS showed the reaction was completed. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl nonanoate (127 mg, 0.177 mmol, 66.2% yield) as a white solid. LCMS (M+H)=703.0; Retention time (10 mM NH₄HCO₃)=1.80 min. ¹H NMR (400 MHz, CDCl₃) δ 8.06 (s, 1H), 7.31-7.27 (m, 2H), 7.21 (d, J=8.8 Hz, 2H), 7.13 (t, J=7.2 Hz, 1H), 6.43 (t, J=6.4 Hz, 1H), 6.05 (brs, 2H), 5.65 (dd, J=7.2, 4.4 Hz, 1H), 5.01 (quint, J=6.4 Hz, 1H), 4.43 (dd, J=11.2, 6.0 Hz, 1H), 4.35 (dd, J=11.2, 5.6 Hz, 1H), 4.06-3.95 (m, 1H), 3.84 (dd, J=11.2, 9.6 Hz, 1H), 2.72 (quint, J=7.2 Hz, 1H), 2.64 (s, 1H), 2.64-2.59 (m, 1H), 2.40 (t, J=7.6 Hz, 2H), 1.67 (quint, J=7.6 Hz, 2H), 1.36 (d, J=6.8 Hz, 3H), 1.35-1.25 (m, 10H), 1.22 (d, J=6.0 Hz, 6H), 0.89 (t, J=6.8 Hz, 3H).

### Example 39

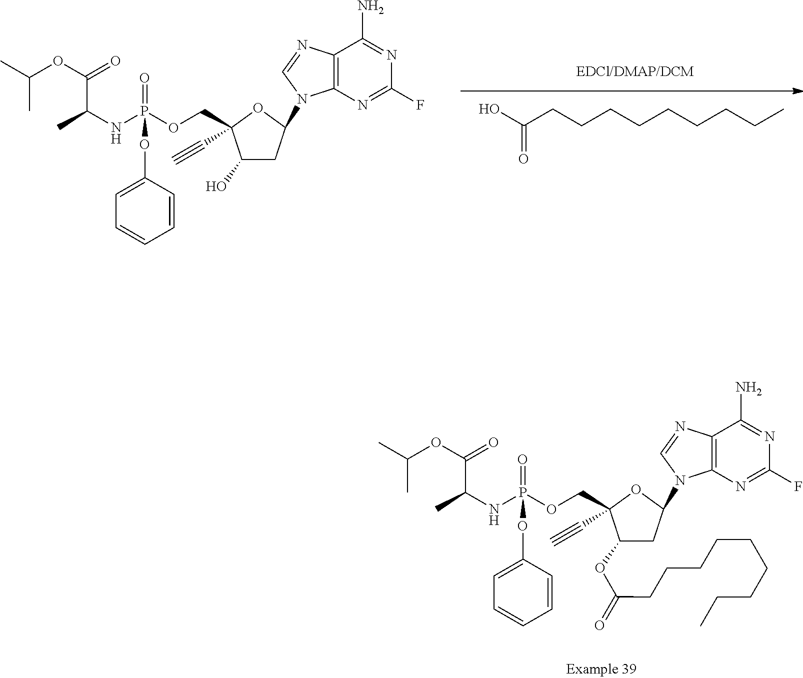 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1O ; CCCCCCCCCC(=O)O ; C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCC]

### (2R,3S,5R)-5-(6-Amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl decanoate

To a solution of decanoic acid (59.7 mg, 0.347 mmol) in DCM (8 mL) was added N,N-dimethylpyridin-4-amine (32.6 mg, 0.267 mmol) and 3-(((ethylimino)methylene)amino)-N,N-dimethylpropan-1-amine hydrochloride (102 mg, 0.533 mmol) and the resulting mixture was stirred for 0.5 h at an ice-water bath temperature. Then, isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.267 mmol) was added and the resulting mixture was stirred at 25° C. for 16 h. The LCMS showed the reaction was completed. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl decanoate (125 mg, 0.172 mmol, 64.5% yield) as a white solid. LCMS (M+H)=717.5; Retention time (0.05% TFA)=2.00 min. ¹H NMR (400 MHz, CDCl₃) δ 8.05 (s, 1H), 7.28 (t, J=8.4 Hz, 2H), 7.20 (d, J=8.8 Hz, 2H), 7.13 (t, J=7.6 Hz, 1H), 6.43 (t, J=6.8 Hz, 1H), 6.04 (brs, 2H), 5.65 (dd, J=6.8, 4.0 Hz, 1H), 5.01 (quint, J=6.4 Hz, 1H), 4.43 (dd, J=10.8, 6.4 Hz, 1H), 4.35 (dd, J=10.8, 6.0 Hz, 1H), 4.06-3.95 (m, 1H), 3.82 (dd, J=11.2, 9.6 Hz, 1H), 2.76-2.67 (m, 1H), 2.66-2.59 (m, 2H), 2.40 (t, J=7.6 Hz, 2H), 1.67 (quint, J=7.6 Hz, 2H), 1.36 (d, J=7.2 Hz, 3H), 1.33-1.24 (m, 12H), 1.22 (d, J=6.4 Hz, 6H), 0.88 (t, J=6.8 Hz, 3H).

### Example 40

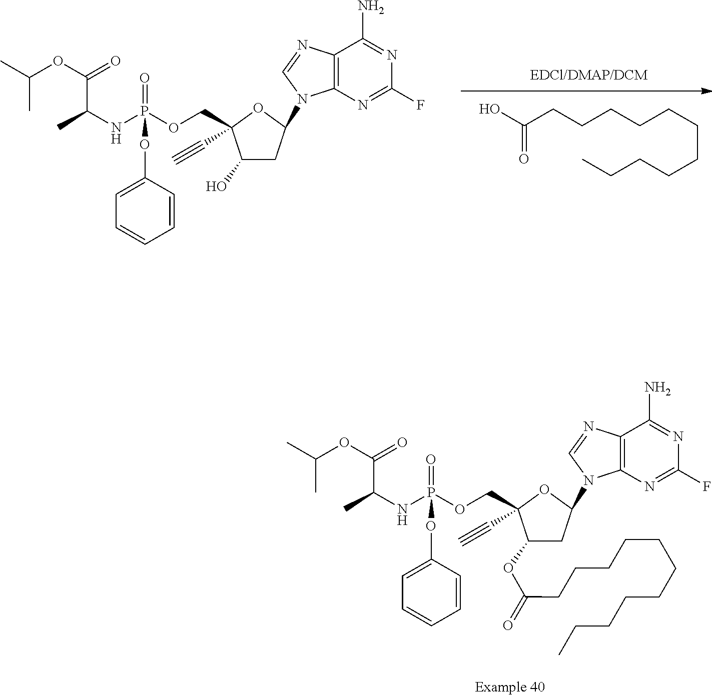 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1O ; CCCCCCCCCCCC(=O)O ; C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCC]

### (2R,3S,5R)-5-(6-Amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl dodecanoate

To a solution of dodecanoic acid (64.1 mg, 0.320 mmol) in DCM (8 mL) was added N,N-dimethylpyridin-4-amine (32.6 mg, 0.267 mmol) and 3-(((ethylimino)methylene)amino)-N,N-dimethylpropan-1-amine hydrochloride (102 mg, 0.533 mmol) and the resulting mixture was stirred for 0.5 h at an ice-water bath temperature. Then, isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.267 mmol) was added and the resulting mixture was stirred at 25° C. for 16 h. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl dodecanoate (79 mg, 0.102 mmol, 38.3% yield) as a white solid. LCMS (M+H)=745.5; Retention time (0.05% TFA)=2.13 min. ¹H NMR (400 MHz, CDCl₃) δ 8.05 (s, 1H), 7.28 (t, J=8.4 Hz, 2H), 7.21 (d, J=8.8 Hz, 2H), 7.13 (t, J=7.2 Hz, 1H), 6.43 (t, J=6.8 Hz, 1H), 5.94 (brs, 2H), 5.65 (dd, J=6.8, 4.0 Hz, 1H), 5.01 (quint, J=6.4 Hz, 1H), 4.43 (dd, J=11.2, 6.4 Hz, 1H), 4.35 (dd, J=11.2, 6.0 Hz, 1H), 4.06-3.95 (m, 1H), 3.79 (dd, J=11.2, 9.6 Hz, 1H), 2.72 (quint, J=6.8 Hz, 1H), 2.64 (s, 1H), 2.63-2.59 (m, 1H), 2.40 (t, J=7.6 Hz, 2H), 1.66-1.62 (m, 2H), 1.36 (d, J=6.8 Hz, 3H), 1.34-1.24 (m, 16H), 1.22 (d, J=6.4 Hz, 6H), 0.88 (t, J=6.8 Hz, 3H).

### Example 41

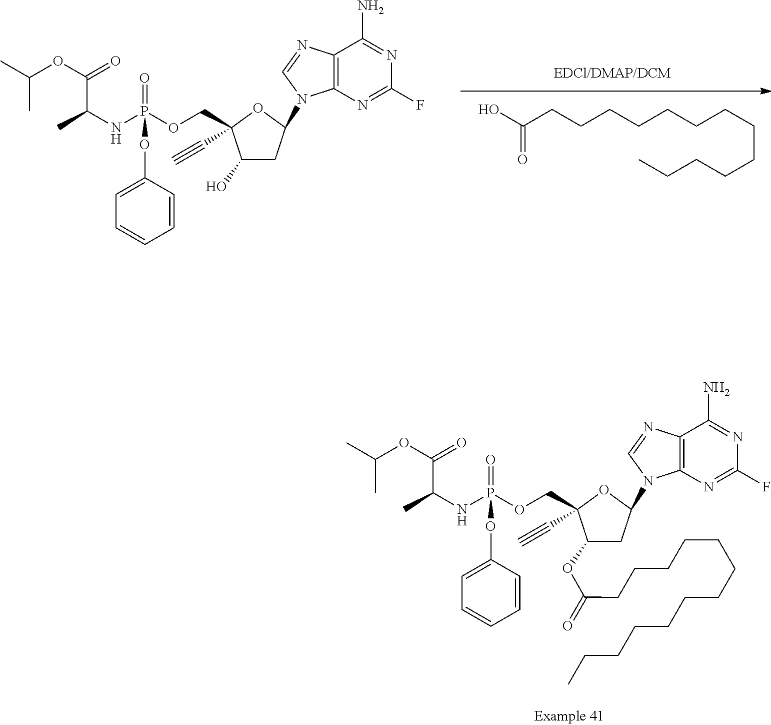 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1O ; CCCCCCCCCCCCCC(=O)O ; C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCCCC]

### (2R,3S,5R)-5-(6-Amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl tetradecanoate

To a solution of tetradecanoic acid (73.1 mg, 0.320 mmol) in DCM (8 mL) was added N,N-dimethylpyridin-4-amine (32.6 mg, 0.267 mmol) and 3-(((ethylimino)methylene)amino)-N,N-dimethylpropan-1-amine hydrochloride (102 mg, 0.533 mmol) and the resulting mixture was stirred for 0.5 h at an ice-water bath temperature. Then, isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.267 mmol) was added and the resulting mixture was stirred at 25° C. for 16 h. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl tetradecanoate (115 mg, 0.147 mmol, 55.3% yield) as a white solid. LCMS (M+H)=773.5; Retention time (0.05% TFA)=2.30 min. ¹H NMR (400 MHz, CDCl₃) δ 8.06 (s, 1H), 7.31-7.27 (m, 2H), 7.21 (d, J=8.4 Hz, 2H), 7.13 (t, J=7.2 Hz, 1H), 6.43 (t, J=6.8 Hz, 1H), 6.06 (brs, 2H), 5.65 (dd, J=6.8, 4.0 Hz, 1H), 5.01 (quint, J=6.4 Hz, 1H), 4.43 (dd, J=10.8, 6.0 Hz, 1H), 4.35 (dd, J=10.8, 5.6 Hz, 1H), 4.05-3.96 (m, 1H), 3.84 (dd, J=11.2, 9.6 Hz, 1H), 2.72 (quint, J=6.8 Hz, 1H), 2.64 (s, 1H), 2.64-2.59 (m, 1H), 2.40 (t, J=7.6 Hz, 2H), 1.67 (quint, J=7.2 Hz, 2H), 1.36 (d, J=7.2 Hz, 3H), 1.33-1.25 (m, 20H), 1.22 (d, J=6.4 Hz, 6H), 0.88 (t, J=6.8 Hz, 3H).

### Example 42

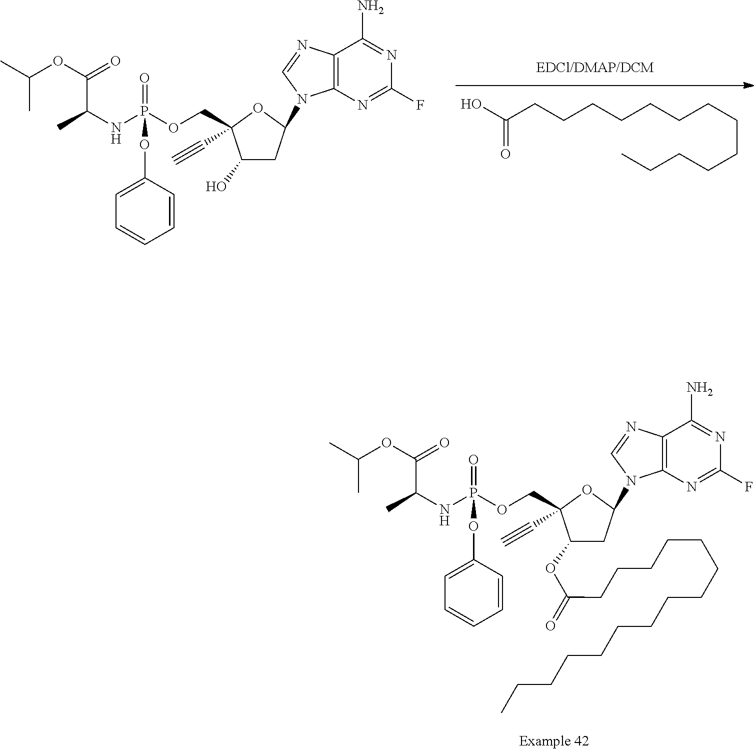 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1O ; CCCCCCCCCCCCCC(=O)O ; C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCCCCCC]

### (2R,3S,5R)-5-(6-Amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl palmitate

To a solution of palmitic acid (82 mg, 0.320 mmol) in DCM (8 mL) was added N,N-dimethylpyridin-4-amine (32.6 mg, 0.267 mmol) and 3-(((ethylimino)methylene)amino)-N,N-dimethylpropan-1-amine hydrochloride (102 mg, 0.533 mmol) and the resulting mixture was stirred for 0.5 h at an ice-water bath temperature. Then, isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.267 mmol) was added and the resulting mixture was stirred at 25° C. for 16 h. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl palmitate (114 mg, 0.141 mmol, 52.9% yield) as a white solid. LCMS (M+H)=801.5; Retention time (0.05% TFA)=2.53 min. ¹H NMR (400 MHz, CDCl₃) δ 8.06 (s, 1H), 7.31-7.27 (m, 2H), 7.21 (d, J=8.4 Hz, 2H), 7.13 (t, J=7.2 Hz, 1H), 6.43 (t, J=6.8 Hz, 1H), 6.14 (brs, 2H), 5.65 (dd, J=6.8, 4.0 Hz, 1H), 5.01 (quint, J=6.0 Hz, 1H), 4.43 (dd, J=10.8, 6.0 Hz, 1H), 4.35 (dd, J=11.2, 6.0 Hz, 1H), 4.06-3.96 (m, 1H), 3.87 (dd, J=11.2, 9.6 Hz, 1H), 2.71 (quint, J=6.8 Hz, 1H), 2.66-2.59 (m, 2H), 2.40 (t, J=7.6 Hz, 2H), 1.66 (quint, J=7.2 Hz, 2H), 1.38 (d, J=6.8 Hz, 3H), 1.32-1.25 (m, 24H), 1.21 (d, J=6.4 Hz, 6H), 0.88 (t, J=6.8 Hz, 3H).

### Example 43

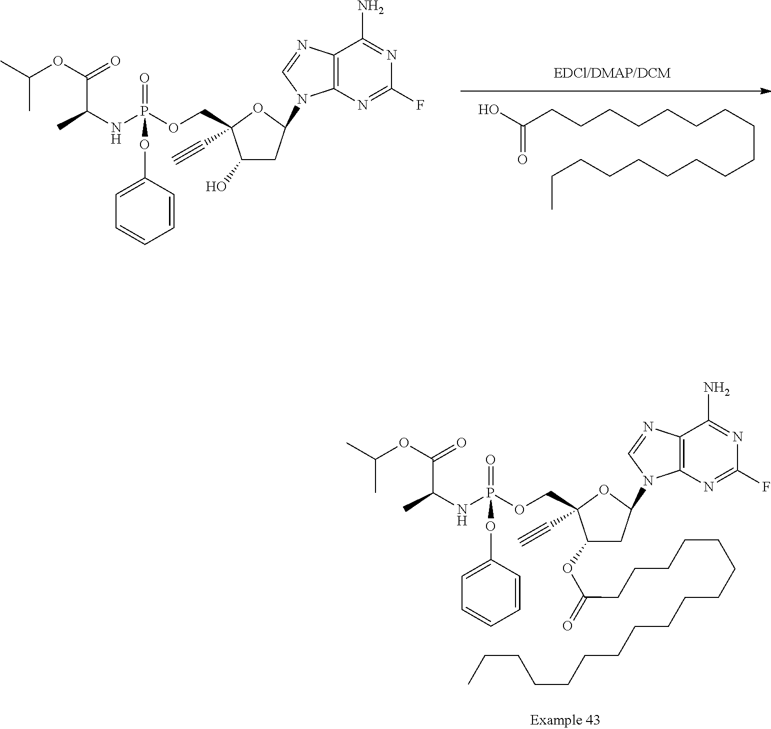 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1O ; CCCCCCCCCCCCCCCCCC(=O)O ; C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCCCCCCCC]

### (2R,3S,5R)-5-(6-Amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl stearate

To a solution of stearic acid (182 mg, 0.640 mmol) in DCM (8 mL) was added N,N-dimethylpyridin-4-amine (65.2 mg, 0.533 mmol) and 3-(((ethylimino)methylene)amino)-N,N-dimethylpropan-1-amine hydrochloride (204 mg, 1.067 mmol) and the resulting mixture was stirred for 0.5 h at an ice-water bath temperature. Then, isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (300 mg, 0.533 mmol) was added and the resulting mixture was stirred at 25° C. for 16 h. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl stearate (126 mg, 0.147 mmol, 27.6% yield) as a white solid. LCMS (M+H)=829.4; Retention time (0.05% TFA)=2.83 min. ¹H NMR (400 MHz, CDCl₃) δ 8.07 (s, 1H), 7.31-7.27 (m, 2H), 7.21 (d, J=8.8 Hz, 2H), 7.13 (t, J=7.2 Hz, 1H), 6.44 (t, J=6.8 Hz, 1H), 6.09 (brs, 2H), 5.65 (dd, J=6.8, 4.0 Hz, 1H), 5.01 (quint, J=6.4 Hz, 1H), 4.43 (dd, J=10.8, 6.0 Hz, 1H), 4.35 (dd, J=11.2, 6.0 Hz, 1H), 4.06-3.96 (m, 1H), 3.84 (dd, J=11.2, 9.6 Hz, 1H), 2.71 (quint, J=6.8 Hz, 1H), 2.66-2.59 (m, 2H), 2.40 (t, J=7.6 Hz, 2H), 1.66 (quint, J=7.2 Hz, 2H), 1.37 (d, J=7.2 Hz, 3H), 1.32-1.25 (m, 28H), 1.22 (d, J=6.0 Hz, 6H), 0.88 (t, J=6.8 Hz, 3H).

### Example 44

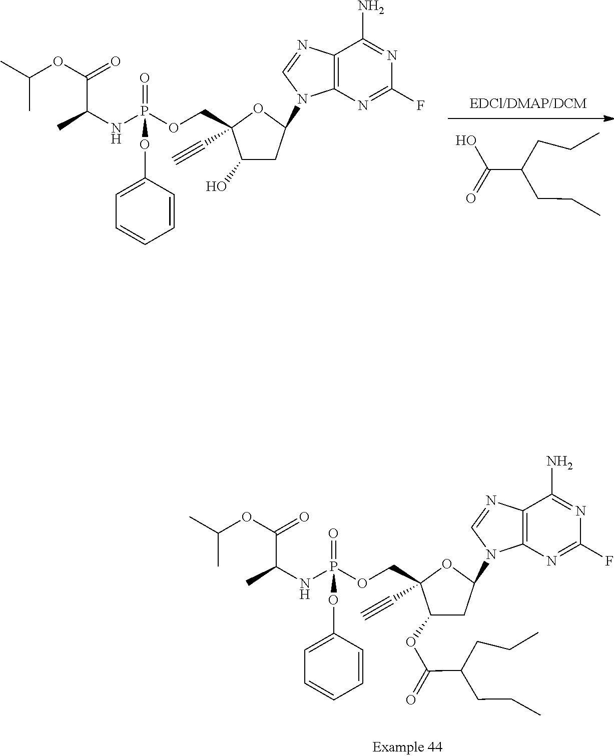

### (2R,3S,5R)-5-(6-Amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl 2-propylpentanoate

To a solution of 2-propylpentanoic acid (46.1 mg, 0.320 mmol) in DCM (8 mL) was added N,N-dimethylpyridin-4-amine (32.6 mg, 0.267 mmol) and 3-(((ethylimino)methylene)amino)-N,N-dimethylpropan-1-amine hydrochloride (102 mg, 0.533 mmol) and the resulting mixture was stirred for 0.5 h at an ice-water bath temperature. Then, isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.267 mmol) was added and the resulting mixture was stirred at 25° C. for 16 h. The LCMS showed the reaction was completed. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-ethynyl-2-((((S)-(((S)-1-isopropoxy-1-oxopropan-2-yl)amino)(phenoxy)phosphoryl)oxy)methyl)tetrahydrofuran-3-yl 2-propylpentanoate (105 mg, 0.150 mmol, 56.1% yield) as a white solid. LCMS (M+H)=689.0; Retention time (10 mM NH₄HCO₃)=1.71 min. ¹H NMR (400 MHz, CDCl₃) δ 8.04 (s, 1H), 7.31-7.27 (m, 2H), 7.20 (d, J=8.8 Hz, 2H), 7.13 (t, J=7.2 Hz, 1H), 6.42 (t, J=6.4 Hz, 1H), 5.87 (brs, 2H), 5.65 (dd, J=6.8, 4.8 Hz, 1H), 5.00 (quint, J=6.4 Hz, 1H), 4.44 (dd, J=11.2, 6.0 Hz, 1H), 4.33 (dd, J=11.2, 5.6 Hz, 1H), 4.05-3.96 (m, 1H), 3.79 (dd, J=11.2, 9.6 Hz, 1H), 2.80 (quint, J=6.8 Hz, 1H), 2.68-2.60 (m, 2H), 2.51-2.43 (m, 1H), 1.71-1.65 (m, 2H), 1.52-1.44 (m, 2H), 1.36 (d, J=7.2 Hz, 3H), 1.35-1.27 (m, 4H), 1.21 (d, J=6.0 Hz, 6H), 0.91 (t, J=7.2 Hz, 6H).

### Example 45

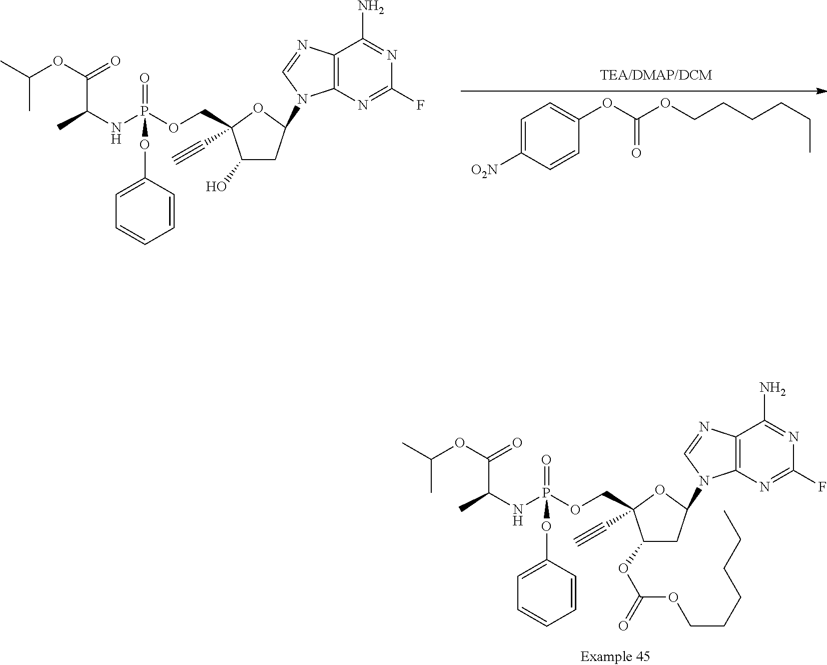 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1O ; CCCCCCOC(=O)Oc1ccc([N+](=O)[O-])cc1 ; C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OCCCCCC]

Isopropyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-alaninate A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (180 mg, 0.320 mmol) and DMAP (39.1 mg, 0.320 mmol) in DCM (6 mL) was treated with TEA (0.134 mL, 0.960 mmol followed by hexyl (4-nitrophenyl) carbonate (428 mg, 1.600 mmol) and the mixture was stirred at 25° C. for 2 days. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) to give isopropyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-alaninate (105 mg, 0.145 mmol, 45.4% yield) as a white solid. LCMS (M+H)=691.3; Retention time (0.05% TFA)=1.81 min. ¹H NMR (400 MHz, CDCl₃) δ 8.01 (s, 1H), 7.31-7.27 (m, 2H), 7.20 (d, J=8.4 Hz, 2H), 7.13 (t, J=7.2 Hz, 1H), 6.42 (t, J=6.4 Hz, 1H), 5.97 (brs, 2H), 5.57 (dd, J=7.2, 4.4 Hz, 1H), 5.00 (quint, J=6.4 Hz, 1H), 4.46 (dd, J=11.2, 6.4 Hz, 1H), 4.37 (dd, J=11.2, 6.4 Hz, 1H), 4.19 (dt, J=6.8, 2.8 Hz, 2H), 4.06-3.95 (m, 1H), 3.83 (dd, J=11.2, 9.6 Hz, 1H), 2.85 (quint, J=7.2 Hz, 1H), 2.76-2.70 (m, 1H), 2.69 (s, 1H), 1.70 (quint, J=6.8 Hz, 2H), 1.36 (d, J=7.2 Hz, 3H), 1.36-1.24 (m, 6H), 1.21 (d, J=6.4 Hz, 6H), 0.90 (t, J=6.8 Hz, 3H).

### Example 46

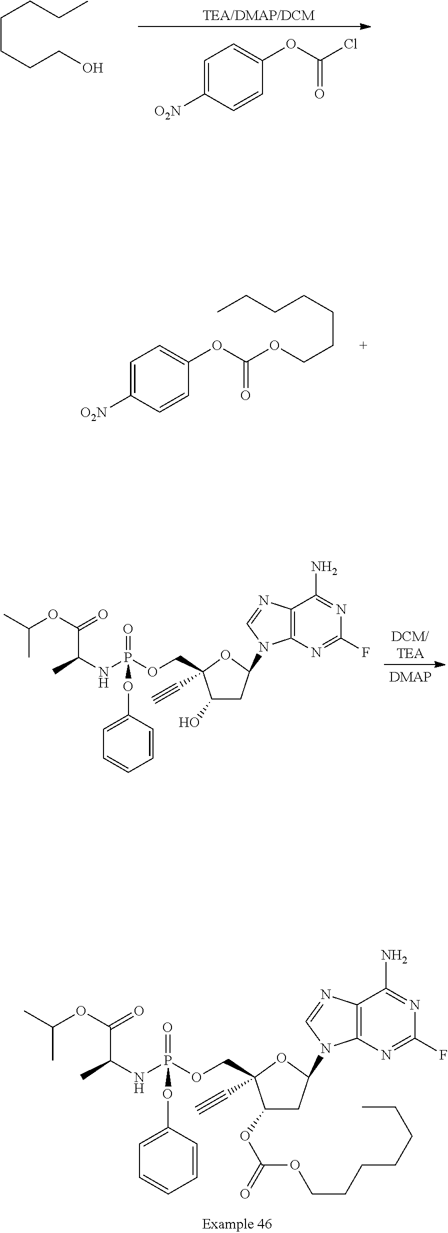

### Step 1: Heptyl (4-nitrophenyl) carbonate

To a mixture of heptan-1-ol (4 g, 34.4 mmol) and DMAP (0.421 g, 3.44 mmol) in DCM (80 mL) was added TEA (7.20 mL, 51.6 mmol) dropwise at 0° C. After stirring for 15 min, 4-nitrophenyl carbonochloridate (8.16 g, 34.4 mmol) was added. The resulting reaction mixture was stirred at 25° C. for 16 h. TLC showed the reaction was finished. The reaction mixture was partitioned between DCM (100 mL) and H₂O (50 mL). The organic layer was washed with brine (40 mL), dried over Na₂SO₄, filtered and concentrated to obtain the heptyl (4-nitrophenyl) carbonate (9 g, 28.8 mmol, 84% yield) as yellow oil. ¹H NMR (400 MHz, CDCl₃) δ 8.28 (dd, J=7.2, 2.4 Hz, 2H), 7.39 (dd, J=7.2, 2.0 Hz, 2H), 4.29 (t, J=6.8 Hz, 2H), 1.76 (quint, J=6.8 Hz, 2H), 1.46-1.27 (m, 8H), 0.90 (t, J=6.8 Hz, 3H).

### Step 2: Isopropyl ((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)-L-alaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.356 mmol) and DMAP (43.4 mg, 0.356 mmol) in DCM (6 mL) was treated with TEA (0.149 mL, 1.067 mmol) followed by heptyl (4-nitrophenyl) carbonate (500 mg, 1.778 mmol) and the mixture was stirred at 25° C. for 2 days. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) to give isopropyl ((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)-L-alaninate (97 mg, 0.135 mmol, 38.0% yield) as a white solid. LCMS (M+H)=705.4; Retention time (0.05% TFA)=1.87 min. ¹H NMR (400 MHz, CDCl₃) δ 8.06 (s, 1H), 7.28 (t, J=8.0 Hz, 2H), 7.20 (d, J=8.4 Hz, 2H), 7.12 (t, J=6.8 Hz, 1H), 6.42 (t, J=6.8 Hz, 1H), 5.97 (brs, 2H), 5.56 (dd, J=6.8, 4.0 Hz, 1H), 5.00 (quint, J=6.4 Hz, 1H), 4.46 (dd, J=10.8, 6.4 Hz, 1H), 4.37 (dd, J=11.2, 6.0 Hz, 1H), 4.19 (dt, J=6.8, 2.4 Hz, 2H), 4.06-3.95 (m, 1H), 3.79 (dd, J=11.2, 9.6 Hz, 1H), 2.83 (quint, J=6.8 Hz, 1H), 2.76-2.70 (m, 1H), 2.69 (s, 1H), 1.70 (quint, J=6.8 Hz, 2H), 1.36 (d, J=6.8 Hz, 3H), 1.36-1.25 (m, 8H), 1.22 (d, J=6.0 Hz, 6H), 0.89 (t, J=6.8 Hz, 3H).

### Example 47

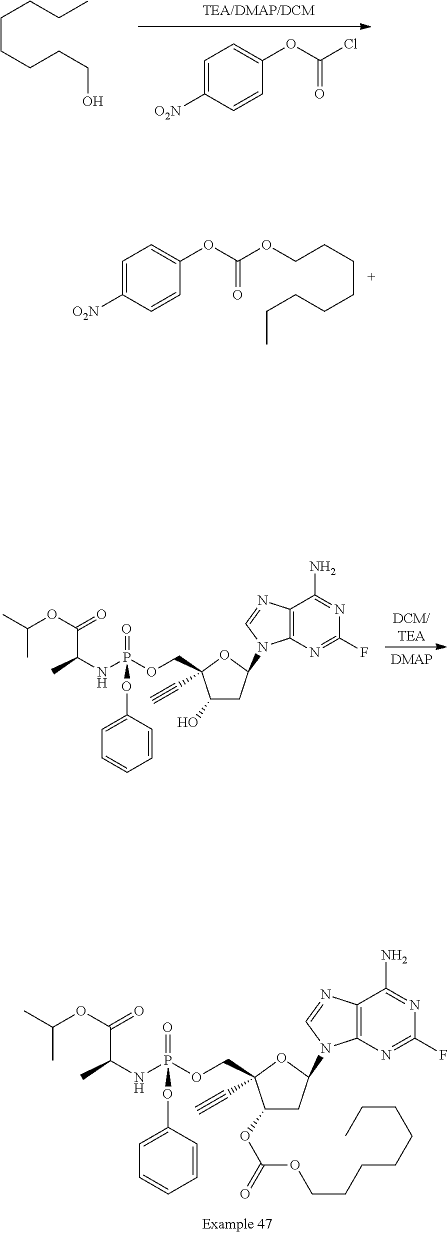

### Step 1: 4-Nitrophenyl octyl carbonate

To a mixture of octan-1-ol (4 g, 30.7 mmol) and DMAP (0.375 g, 3.07 mmol) in DCM (120 mL) was added TEA (4.66 g, 46.1 mmol) dropwise at 0° C. After stirring for 15 min, 4-nitrophenyl carbonochloridate (6.19 g, 30.7 mmol) was added. The resulting reaction mixture was stirred at 25° C. for 16 h. TLC showed the reaction was finished. The reaction mixture was partitioned between DCM (120 mL) and H₂O (50 mL). The organic layer was washed with brine (40 mL), dried over Na₂SO₄, filtered and concentrated to obtain the 4-nitrophenyl octyl carbonate (9 g, 27.4 mmol, 89% yield) as yellow oil. ¹H NMR (400 MHz, CDCl₃) δ 8.28 (dd, J=7.2, 2.4 Hz, 2H), 7.38 (dd, J=7.2, 2.0 Hz, 2H), 4.29 (t, J=6.8 Hz, 2H), 1.76 (quint, J=6.8 Hz, 2H), 1.45-1.24 (m, 10H), 0.89 (t, J=6.8 Hz, 3H).

### Step 2: Isopropyl ((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)-L-alaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.356 mmol) and DMAP (43.4 mg, 0.356 mmol) in DCM (6 mL) was treated with TEA (0.149 mL, 1.067 mmol) followed by 4-nitrophenyl octyl carbonate (525 mg, 1.778 mmol) and the mixture was stirred at 25° C. for 2 days. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) to give isopropyl ((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)-L-alaninate (110 mg, 0.150 mmol, 42.1% yield) as a white solid. LCMS (M+H)=719.2; Retention time (0.05% TFA)=1.92 min. ¹H NMR (400 MHz, CDCl₃) δ 8.01 (s, 1H), 7.31-7.27 (m, 2H), 7.22-7.16 (m, 2H), 7.13 (t, J=7.2 Hz, 1H), 6.42 (t, J=6.8 Hz, 1H), 6.00 (brs, 2H), 5.56 (dd, J=7.2, 4.4 Hz, 1H), 5.00 (quint, J=6.4 Hz, 1H), 4.46 (dd, J=11.2, 6.4 Hz, 1H), 4.37 (dd, J=11.2, 6.0 Hz, 1H), 4.19 (dt, J=6.8, 2.4 Hz, 2H), 4.06-3.97 (m, 1H), 3.82 (dd, J=11.2, 9.6 Hz, 1H), 2.85 (quint, J=6.8 Hz, 1H), 2.76-2.70 (m, 1H), 2.69 (s, 1H), 1.70 (quint, J=7.2 Hz, 2H), 1.36 (d, J=7.2 Hz, 3H), 1.34-1.24 (m, 10H), 1.21 (d, J=6.0 Hz, 6H), 0.89 (t, J=6.8 Hz, 3H).

### Example 48

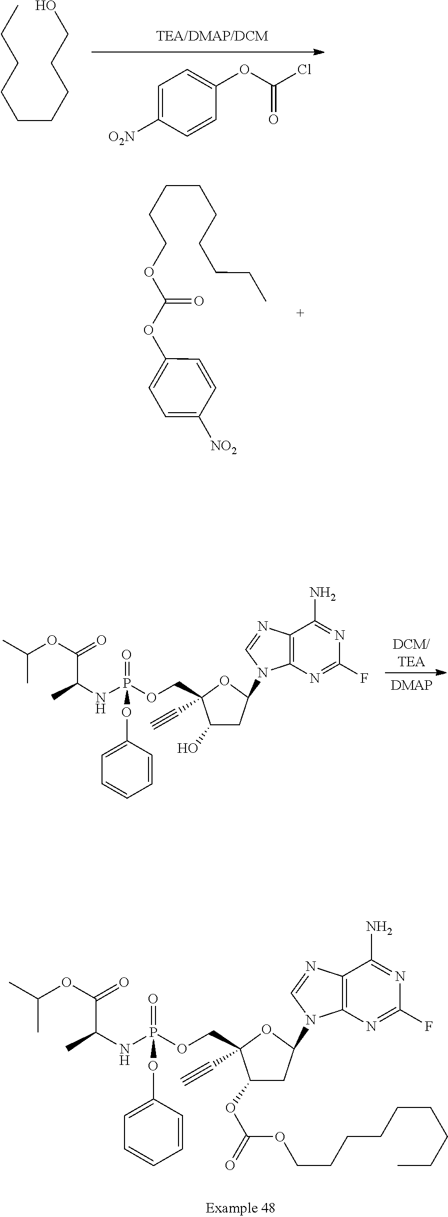

### Step 1: 4-Nitrophenyl nonyl carbonate

To a solution of 4-nitrophenyl carbonochloridate (5 g, 24.81 mmol) and nonan-1-ol (3.58 g, 24.81 mmol), DMAP (0.303 g, 2.481 mmol) in DCM (100 mL) was treated with TEA (5.19 mL, 37.2 mmol) and the mixture was stirred at RT for 3 h. TLC showed the reaction was completed. Water (80 mL) was added, the mixture was extracted with DCM (100 mL×2). The combined organic phases were washed with brine, dried over Na₂SO₄ and concentrated to give 4-nitrophenyl nonyl carbonate (5 g, 13.74 mmol, 55.4% yield) as yellow oil. ¹H NMR (400 MHz, CDCl₃) δ 8.39-8.22 (m, 2H), 7.46-7.31 (m, 2H), 4.29 (t, J=6.7 Hz, 2H), 1.88-1.72 (m, 2H), 1.30 (d, J=11.3 Hz, 12H), 0.90-0.85 (m, 3H).

### Step 2: Isopropyl ((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)-L-alaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.356 mmol) and DMAP (43.4 mg, 0.356 mmol) in DCM (5 mL) was treated with TEA (0.149 mL, 1.067 mmol) followed by 4-nitrophenyl nonyl carbonate (550 mg, 1.778 mmol) and the mixture was stirred at 25° C. for 2 days. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) give isopropyl ((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)-L-alaninate (115 mg, 0.150 mmol, 42.1% yield) as a white solid. LCMS (M+H)=733.3; Retention time (0.05% TFA)=1.98 min. ¹H NMR (400 MHz, CDCl₃) δ 8.01 (s, 1H), 7.31-7.27 (m, 2H), 7.22-7.18 (m, 2H), 7.12 (t, J=7.2 Hz, 1H), 6.42 (t, J=6.8 Hz, 1H), 6.00 (brs, 2H), 5.56 (dd, J=6.8, 4.0 Hz, 1H), 5.00 (quint, J=6.4 Hz, 1H), 4.46 (dd, J=11.2, 6.4 Hz, 1H), 4.37 (dd, J=11.2, 6.0 Hz, 1H), 4.19 (dt, J=6.8, 2.4 Hz, 2H), 4.07-3.96 (m, 1H), 3.83 (dd, J=11.2, 9.6 Hz, 1H), 2.85 (quint, J=6.8 Hz, 1H), 2.76-2.70 (m, 1H), 2.68 (s, 1H), 1.70 (quint, J=7.2 Hz, 2H), 1.36 (d, J=7.2 Hz, 3H), 1.34-1.25 (m, 12H), 1.21 (d, J=6.0 Hz, 6H), 0.88 (t, J=6.8 Hz, 3H).

### Example 49

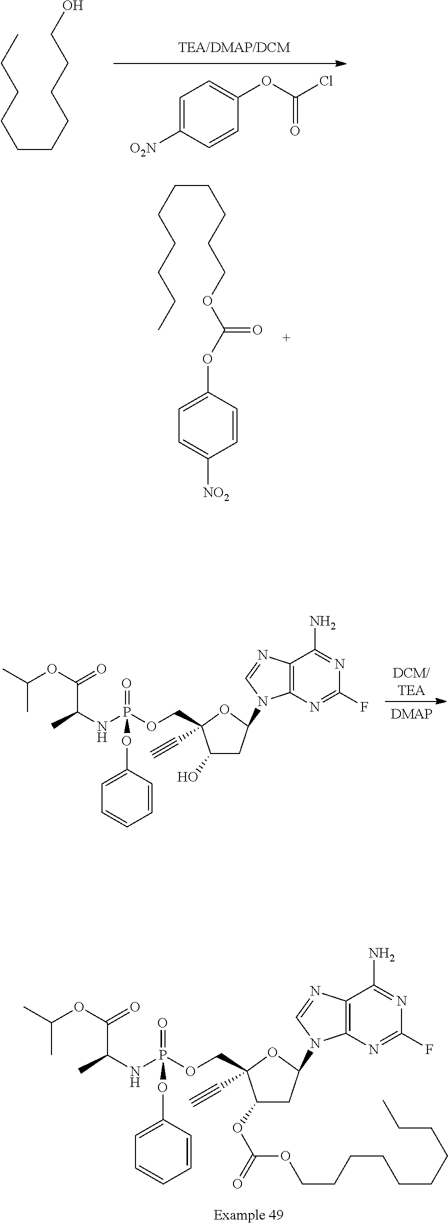

### Step 1: Decyl (4-nitrophenyl) carbonate

To a solution of 4-nitrophenyl carbonochloridate (5 g, 24.81 mmol) and decan-1-ol (3.93 g, 24.81 mmol), DMAP (0.303 g, 2.481 mmol) in DCM (100 mL) was treated with TEA (5.19 mL, 37.2 mmol) and the mixture was stirred at RT for 3 h. TLC showed the reaction was completed. Water (80 mL) was added, the mixture was extracted with DCM (100 mL×2). The combined organic phases were washed with brine, dried over Na₂SO₄ and concentrated to give decyl (4-nitrophenyl) carbonate (5.1 g, 13.40 mmol, 54.0% yield) as yellow oil. ¹H NMR (400 MHz, CDCl₃) δ 8.39-8.22 (m, 2H), 7.46-7.31 (m, 2H), 4.29 (t, J=6.7 Hz, 2H), 1.88-1.72 (m, 2H), 1.30 (d, J=11.3 Hz, 14H), 0.90-0.85 (m, 3H).

### Step 2: Isopropyl ((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)-L-alaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.356 mmol) and DMAP (43.4 mg, 0.356 mmol) in DCM (5 mL) was treated with TEA (0.149 mL, 1.067 mmol) followed by decyl (4-nitrophenyl) carbonate (575 mg, 1.778 mmol) and the mixture was stirred at 25° C. for 2 days. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) to give isopropyl ((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)-L-alaninate (139 mg, 0.180 mmol, 50.7% yield) as a white solid. LCMS (M+H)=747.5; Retention time (0.05% TFA)=2.04 min. ¹H NMR (400 MHz, CDCl₃) δ 8.02 (s, 1H), 7.28 (t, J=8.4 Hz, 2H), 7.20 (d, J=8.8 Hz, 2H), 7.12 (t, J=6.8 Hz, 1H), 6.42 (t, J=6.8 Hz, 1H), 6.09 (brs, 2H), 5.56 (dd, J=7.2, 4.4 Hz, 1H), 5.00 (quint, J=6.4 Hz, 1H), 4.46 (dd, J=11.2, 6.4 Hz, 1H), 4.37 (dd, J=11.2, 9.6 Hz, 1H), 4.19 (dt, J=6.8, 2.0 Hz, 2H), 4.05-3.97 (m, 1H), 3.88 (dd, J=11.2, 6.4 Hz, 1H), 2.85 (quint, J=7.2 Hz, 1H), 2.76-2.70 (m, 1H), 2.68 (s, 1H), 1.70 (quint, J=6.8 Hz, 2H), 1.36 (d, J=7.2 Hz, 3H), 1.34-1.24 (m, 14H), 1.21 (d, J=6.4 Hz, 6H), 0.88 (t, J=6.8 Hz, 3H).

### Example 50

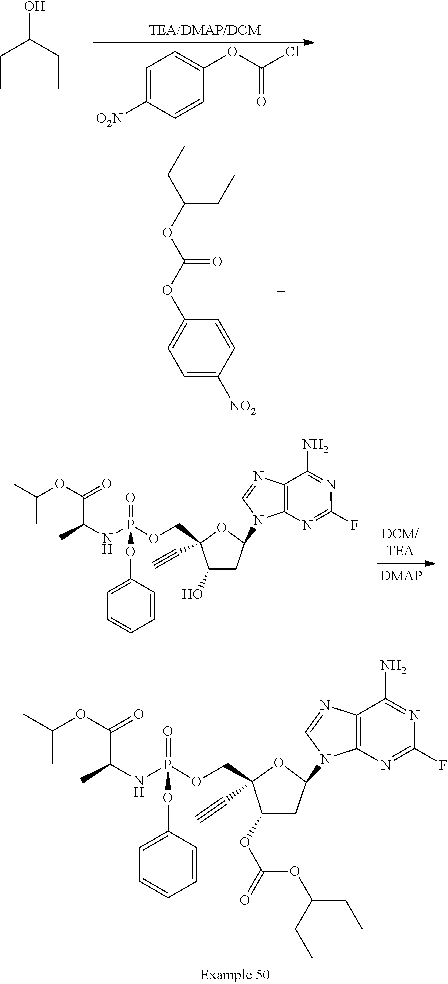

### Step 1: 4-Nitrophenyl pentan-3-yl carbonate

A mixture of 4-nitrophenyl carbonochloridate (5 g, 24.81 mmol) and pentan-3-ol (2.187 g, 24.81 mmol) in DCM (100 mL) was treated with TEA (5.19 mL, 37.2 mmol) and the mixture 10 was stirred at RT for 3 h. TLC showed the new product formed. Water (80 mL) was added, the mixture was extracted with DCM (100 mL×2). The combined organic phases were washed with brine, dried over Na₂SO₄ and concentrated to afford the 4-nitrophenyl pentan-3-yl carbonate (6 g, 23.69 mmol, 96% yield). ¹H NMR (400 MHz, CDCl₃) δ 8.28 (dd, J=7.2, 2.0 Hz, 2H), 7.39 (dd, J=7.2, 2.0 Hz, 2H), 4.90-4.83 (m, 1H), 1.75-1.57 (m, 2H), 1.50-1.36 (m, 2H), 0.96 (t, J=7.2 Hz, 6H).

### Step 2: Isopropyl ((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)-L-alaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.356 mmol) and DMAP (43.4 mg, 0.356 mmol) in DCM (6 mL) was treated with TEA (0.149 mL, 1.067 mmol) followed by 4-nitrophenyl pentan-3-yl carbonate (360 mg, 1.422 mmol) and the mixture was stirred at 25° C. for 2 days. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) to give isopropyl ((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)-L-alaninate (134 mg, 0.193 mmol, 54.4% yield) as a white solid. LCMS (M+H)=677.2; Retention time (0.05% TFA)=1.73 min. ¹H NMR (400 MHz, CDCl₃) δ 8.02 (s, 1H), 7.30-7.25 (m, 2H), 7.22-7.18 (m, 2H), 7.12 (t, J=7.2 Hz, 1H), 6.43 (t, J=6.8 Hz, 1H), 6.06 (brs, 2H), 5.56 (dd, J=7.2, 4.0 Hz, 1H), 5.00 (quint, J=6.4 Hz, 1H), 4.63 (quint, J=6.4 Hz, 1H), 4.47 (dd, J=11.2, 6.4 Hz, 1H), 4.39 (dd, J=10.8, 6.0 Hz, 1H), 4.07-3.97 (m, 1H), 3.86 (dd, J=11.2, 9.6 Hz, 1H), 2.83 (quint, J=7.2 Hz, 1H), 2.71 (ddd, J=14.0, 6.4, 4.0 Hz, 1H), 2.67 (s, 1H), 1.66 (quint, J=6.8 Hz, 4H), 1.36 (d, J=6.8 Hz, 3H), 1.21 (d, J=6.4 Hz, 6H), 0.94 (dt, J=7.6, 1.2 Hz, 6H).

### Example 51

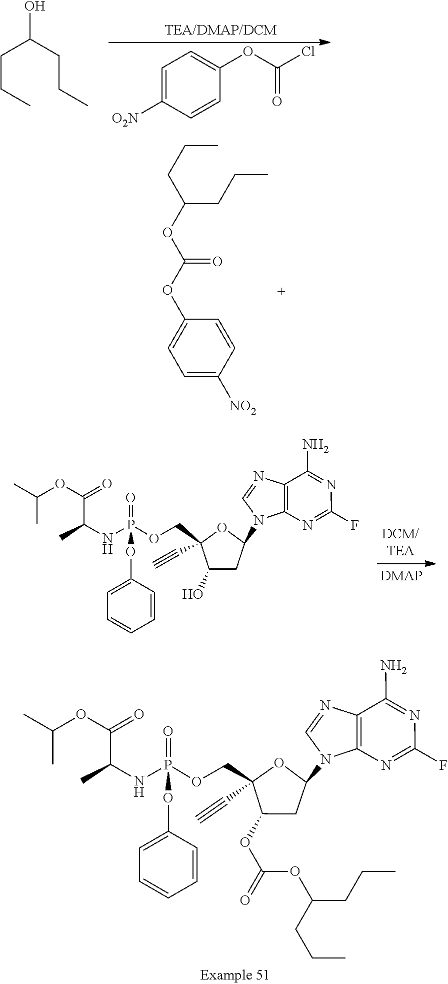

### Step 1: Heptan-4-yl (4-nitrophenyl) carbonate

To a mixture of heptan-4-ol (3 g, 25.8 mmol) and DMAP (0.315 g, 2.58 mmol) in DCM (120 mL) was added TEA (5.38 mL, 38.7 mmol) dropwise at 0° C. After stirring for 15 min, 4-nitrophenyl carbonochloridate (5.20 g, 25.8 mmol) was added. The resulting reaction mixture was stirred at 25° C. for 16 h. TLC showed the reaction was finished. The reaction mixture was partitioned between DCM (120 mL) and H₂O (50 mL). The organic layer was washed with brine (40 mL), dried over Na₂SO₄, filtered and concentrated. The residue was purified by silica gel column chromatography (hexane/ethyl acetate=10/1) to obtain the heptan-4-yl (4-nitrophenyl) carbonate (3.7 g, 13.15 mmol, 50.9% yield) as a colorless oil. ¹H NMR (400 MHz, CDCl₃) δ 8.28 (dd, J=7.2, 2.0 Hz, 2H), 7.39 (dd, J=7.2, 2.0 Hz, 2H), 4.90-4.83 (m, 1H), 1.75-1.57 (m, 4H), 1.50-1.36 (m, 4H), 0.96 (t, J=7.2 Hz, 6H).

### Step 2: Isopropyl ((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)-L-alaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.356 mmol) and DMAP (43.4 mg, 0.356 mmol) in DCM (6 mL) was treated with TEA (0.149 mL, 1.067 mmol) followed by heptan-4-yl (4-nitrophenyl) carbonate (400 mg, 1.422 mmol) and the mixture was stirred at 25° C. for 2 days. LCMS showed the reaction was finished. The reaction mixture was concentrated and the residue was dissolved in MeOH and purified by reverse phase chromatography (SepaFlash@ C18 column, Biotage; gradient: 0%-100% ACN/10 mM aq. NH₄HCO₃); flow rate: 40 mL/min; column: Boston; 40 g) to give isopropyl ((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)-L-alaninate (139 mg, 0.195 mmol, 54.8% yield) as a white solid. LCMS (M+H)=705.3; Retention time (0.05% TFA)=1.84 min. ¹H NMR (400 MHz, CDCl₃) δ 8.01 (s, 1H), 7.31-7.27 (m, 2H), 7.20 (d, J=8.4 Hz, 2H), 7.12 (t, J=7.2 Hz, 1H), 6.43 (t, J=6.8 Hz, 1H), 5.92 (brs, 2H), 5.55 (dd, J=6.8, 4.0 Hz, 1H), 5.00 (quint, J=6.4 Hz, 1H), 4.81-4.74 (m, 1H), 4.47 (dd, J=10.8, 6.0 Hz, 1H), 4.39 (dd, J=11.2, 6.0 Hz, 1H), 4.06-3.97 (m, 1H), 3.80 (dd, J=10.8, 9.6 Hz, 1H), 2.84 (quint, J=7.2 Hz, 1H), 2.75-2.68 (m, 1H), 2.66 (s, 1H), 1.66-1.51 (m, 4H), 1.46-1.31 (m, 7H), 1.22 (d, J=6.4 Hz, 6H), 0.93 (dt, J=12.0, 7.2 Hz, 6H).

### Example 52

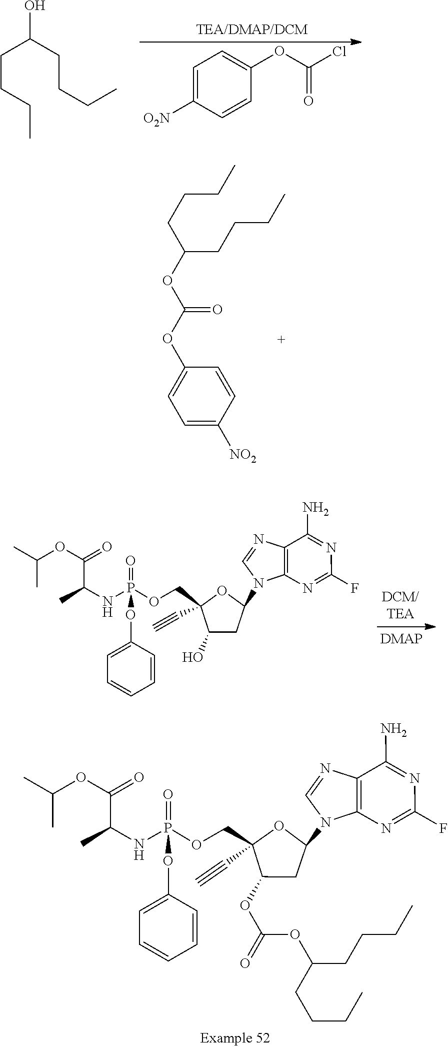

### Step 1: 4-Nitrophenyl nonan-5-yl carbonate

To a mixture of nonan-5-ol (3 g, 20.80 mmol) and DMAP (0.254 g, 2.080 mmol) in DCM (120 mL) was added TEA (3.16 g, 31.2 mmol) dropwise at 0° C. After stirring for 15 min, 4-nitrophenyl carbonochloridate (4.19 g, 20.80 mmol) was added. The resulting reaction mixture was stirred at 25° C. for 16 h. TLC showed the reaction was finished. The reaction mixture was partitioned between DCM (120 mL) and H₂O (50 mL). The organic layer was washed with brine (40 mL), dried over Na₂SO₄, filtered and concentrated. The residue was purified by silica gel column chromatography (hexane/ethyl acetate=10/1) to obtain the 4-nitrophenyl nonan-5-yl carbonate (4.08 g, 13.19 mmol, 63.4% yield) as a colorless oil. ¹H NMR (400 MHz, CDCl₃) δ 8.28 (dd, J=7.2, 2.0 Hz, 2H), 7.39 (dd, J=7.2, 2.0 Hz, 2H), 4.86-4.79 (m, 1H), 1.74-1.62 (m, 4H), 1.43-1.31 (m, 8H), 0.93 (t, J=7.2 Hz, 6H).

### Step 2: Isopropyl ((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)-L-alaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.356 mmol) and DMAP (43.4 mg, 0.356 mmol) in DCM (6 mL) was treated with TEA (0.149 mL, 1.067 mmol) followed by 4-nitrophenyl nonan-5-yl carbonate (440 mg, 1.422 mmol) and the mixture was stirred at 25° C. for 3 days. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) to give isopropyl ((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)-L-alaninate (102 mg, 0.137 mmol, 38.6% yield) as a white solid. LCMS (M+H)=733.3; Retention time (0.05% TFA)=1.94 min. ¹H NMR (400 MHz, CDCl₃) δ 8.02 (s, 1H), 7.31-7.27 (m, 2H), 7.20 (d, J=8.4 Hz, 2H), 7.12 (t, J=7.2 Hz, 1H), 6.43 (t, J=6.8 Hz, 1H), 5.93 (brs, 2H), 5.55 (dd, J=6.8, 4.0 Hz, 1H), 5.00 (quint, J=6.4 Hz, 1H), 4.77-4.71 (m, 1H), 4.47 (dd, J=11.2, 6.4 Hz, 1H), 4.39 (dd, J=11.2, 6.0 Hz, 1H), 4.07-3.95 (m, 1H), 3.80 (dd, J=10.8, 9.6 Hz, 1H), 2.81 (quint, J=7.2 Hz, 1H), 2.71 (ddd, J=14.0, 6.0, 4.0 Hz, 1H), 2.65 (s, 1H), 1.65-1.51 (m, 4H), 1.36 (d, J=7.2 Hz, 3H), 1.35-1.25 (m, 8H), 1.22 (d, J=6.0 Hz, 6H), 0.94-0.88 (m, 6H).

### Example 53

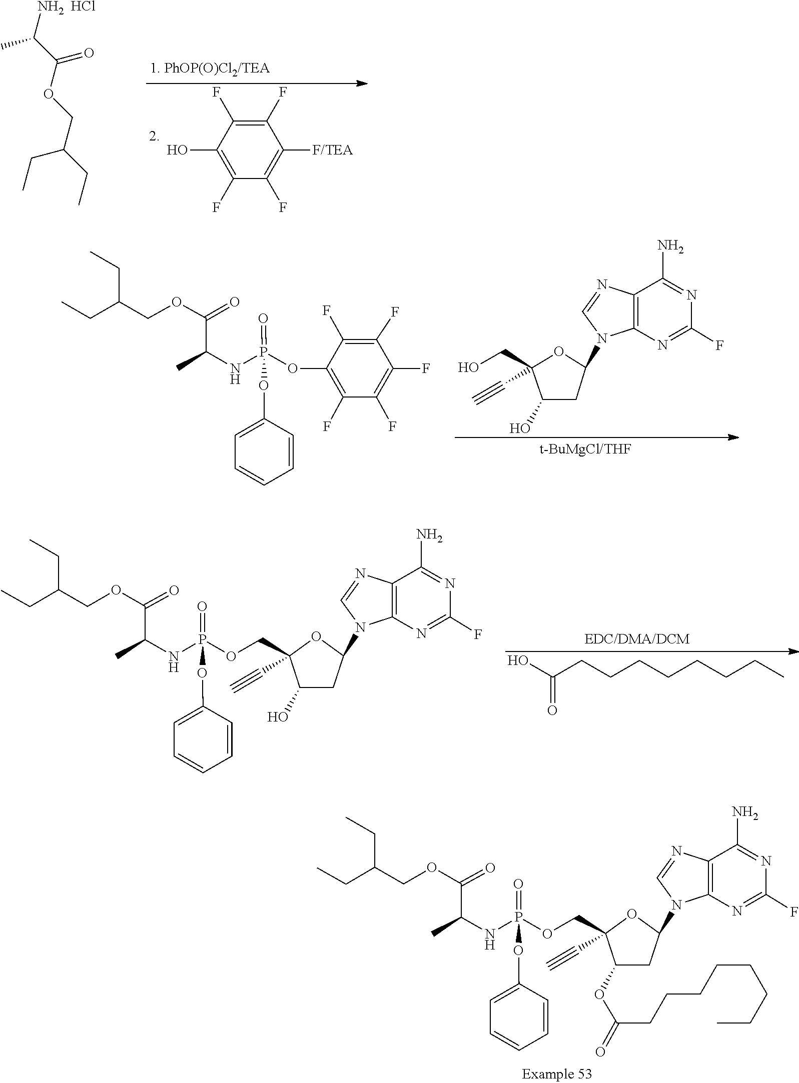

### Step 1: 2-Ethylbutyl ((perfluorophenoxy)(phenoxy)phosphoryl)-L-alaninate

To a stirred solution of 2-ethylbutyl L-alaninate hydrochloride (20 g, 95 mmol) in anhydrous dichloromethane (50 mL) was added dropwise TEA (10.0 g, 13.8 mL, 99.0 mmol) at −70° C. over 15 min. To this mixture was added a solution of phenylphosphonic dichloride (18.41 g, 94 mmol) in anhydrous dichloromethane (80 mL) over 1 h. The reaction mixture was stirred at this temperature for additional 30 min and then allowed to warm to 0° C. over 2 h and stirred for additional 1 h. To this mixture was added a solution of 2,3,4,5,6-pentafluorophenol (17.38 g, 94 mmol) and TEA (5.27 g, 7.26 mL, 52.1 mmol) in dichloromethane (60 mL) over 20 min. The crude mixture was allowed to stir at 0° C. for 16 h. LCMS showed the reaction was completed. The mixture was concentrated under reduced pressure. The residue was triturated with TBME (800 mL) and the triethylamine hydrochloride salt was removed by filtration. The cake was washed with TBME (2×50 mL) and the combined filtrate was concentrated under reduced pressure. The residue was triturated with 20% EtOAc in hexanes (250 mL) and solids collected by filtration to give 2-ethylbutyl ((perfluorophenoxy)(phenoxy)phosphoryl)-L-alaninate (7.8 g, 15.75 mmol, 16.51% yield) as a white solid LCMS: (M+H)=496.1; Retention time (0.05% TFA)=2.01 min.

### Step 2: 2-Ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a solution of (2R,3S,5R)-5-(6-amino-2-fluoro-9H-purin-9-yl)-2-ethynyl-2-(hydroxymethyl)tetrahydrofuran-3-ol (0.25 g, 0.852 mmol) in THF (50 mL) and pyridine (2.500 mL) was added tert-butylmagnesium chloride (1.705 mL, 1.705 mmol) dropwise at −15° C. and stirred at −15° C. for 1 h. Then, to the reaction mixture was added a solution of 2-ethylbutyl ((S)-(perfluorophenoxy)(phenoxy)phosphoryl)-L-alaninate (0.549 g, 1.108 mmol) in THF (15 mL) and the resulting reaction mixture was stirred at −15° C. for 2 h. LCMS showed product/by-product=1/1. The reaction mixture was quenched with NH₄Cl (4 mL) and partitioned between EtOAc (200 mL) and H₂O (30 mL). The organic layer was washed with brine (20 mL), dried over Na₂SO₄, filtered and concentrated in vacuum. The residue was purified by silica gel column chromatography (8% MeOH in DCM) to 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (130 mg, 0.213 mmol, 25.01% yield) as a white solid. LCMS: (M+H)=605.3, Retention time (0.05% TFA)=1.61 min.

### Step 3: (2R,3S,5R)-5-(6-Amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl nonanoate

To a solution of nonanoic acid (51.0 mg, 0.323 mmol) in DCM (6 mL) was added N,N-dimethylpyridin-4-amine (30.3 mg, 0.248 mmol) and 3-(((ethylimino)methylene)amino)-N,N-dimethylpropan-1-amine hydrochloride (95 mg, 0.496 mmol) and the resulting mixture was stirred for 0.5 h at an ice-water bath temperature. Then, (2R,3S,5R)-5-(6-amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl nonanoate (100 mg, 0.130 mmol, 52.2% yield) was added and the resulting mixture was stirred at 25° C. for 16 h. The LCMS showed the reaction was completed. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl nonanoate (100 mg, 0.130 mmol, 52.2% yield) as a white solid. LCMS (M+H)=745.3; Retention time (0.05% TFA)=2.08 min. ¹H NMR (400 MHz, CDCl₃) δ 8.07 (s, 1H), 7.31-7.27 (m, 2H), 7.23-7.19 (m, 2H), 7.13 (dt, J=7.2, 0.8 Hz, 1H), 6.43 (t, J=6.8 Hz, 1H), 6.22 (brs, 2H), 5.65 (dd, J=6.8, 4.0 Hz, 1H), 4.43 (dd, J=11.2, 6.4 Hz, 1H), 4.35 (dd, J=11.2, 6.0 Hz, 1H), 4.11-4.04 (m, 2H), 3.99 (dd, J=10.8, 5.6 Hz, 1H), 3.93 (dd, J=11.2, 9.6 Hz, 1H), 2.71 (quint, J=6.8 Hz, 1H), 2.64 (s, 1H), 2.64-2.59 (m, 1H), 2.40 (t, J=7.6 Hz, 2H), 1.67 (quint, J=7.2 Hz, 2H), 1.50 (quint, J=6.0 Hz, 1H), 1.39 (d, J=7.2 Hz, 3H), 1.37-1.25 (m, 14H), 0.92-0.83 (m, 9H).

### Example 54

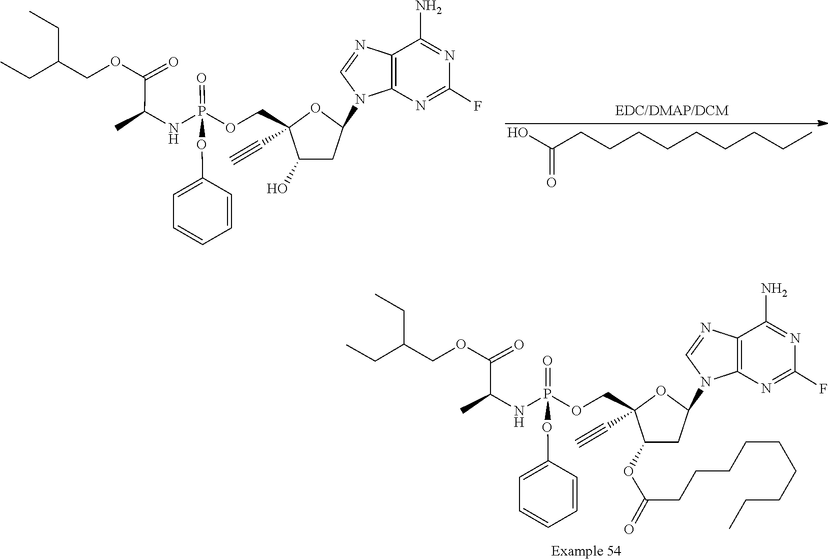 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCCCCCCCC(=O)O ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCC]

### (2R,3S,5R)-5-(6-Amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl decanoate

To a solution of decanoic acid (55.6 mg, 0.323 mmol) in DCM (6 mL) was added N,N-dimethylpyridin-4-amine (30.3 mg, 0.248 mmol) and 3-(((ethylimino)methylene)amino)-N,N-dimethylpropan-1-amine hydrochloride (95 mg, 0.496 mmol) and the resulting mixture was stirred for 0.5 h at an ice-water bath temperature. Then, 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.248 mmol) was added and the resulting mixture was stirred at 25° C. for 16 h. The LCMS showed the reaction was completed. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl decanoate (108 mg, 0.137 mmol, 55.1% yield) as a white solid. LCMS (M+H)=759.4; Retention time (0.05% TFA)=2.15 min. ¹H NMR (400 MHz, CDCl₃) δ 8.13 (s, 1H), 7.31-7.27 (m, 2H), 7.23-7.19 (m, 2H), 7.13 (t, J=7.2 Hz, 1H), 6.43 (t, J=6.8 Hz, 1H), 5.95 (brs, 2H), 5.65 (dd, J=6.8, 4.4 Hz, 1H), 4.43 (dd, J=11.2, 6.4 Hz, 1H), 4.35 (dd, J=10.8, 6.0 Hz, 1H), 4.11-4.04 (m, 2H), 3.99 (dd, J=10.8, 6.0 Hz, 1H), 3.77 (dd, J=11.2, 9.6 Hz, 1H), 2.74-2.60 (m, 3H), 2.40 (t, J=7.6 Hz, 2H), 1.67 (quint, J=7.6 Hz, 2H), 1.51 (quint, J=6.4 Hz, 1H), 1.39 (d, J=6.8 Hz, 3H), 1.36-1.25 (m, 16H), 0.91-0.85 (m, 9H).

### Example 55

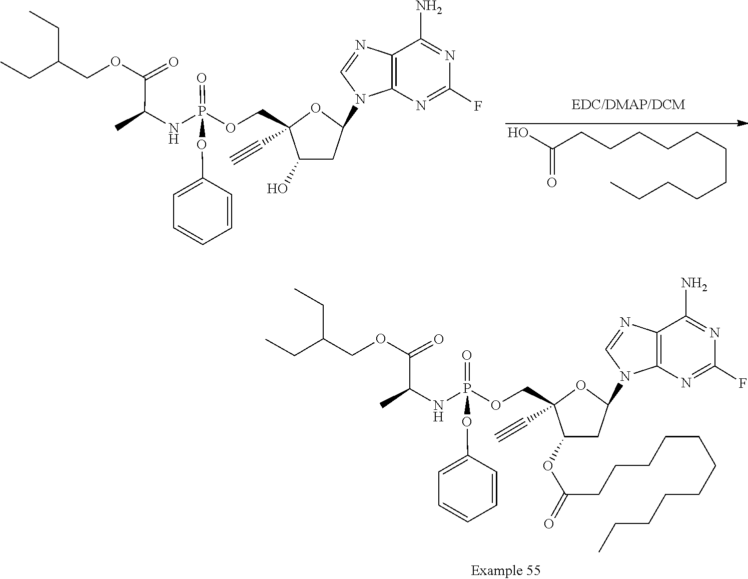 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCCCCCCCCCC(=O)O ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCCCC]

### (2R,3S,5R)-5-(6-Amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl dodecanoate

To a solution of dodecanoic acid (64.6 mg, 0.323 mmol) in DCM (6 mL) was added N,N-dimethylpyridin-4-amine (30.3 mg, 0.248 mmol) and 3-(((ethylimino)methylene)amino)-N,N-dimethylpropan-1-amine hydrochloride (95 mg, 0.496 mmol) and the resulting mixture was stirred for 0.5 h at an ice-water bath temperature. Then, 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.248 mmol) was added and the resulting mixture was stirred at 25° C. for 16 h. The LCMS showed the reaction was completed. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl dodecanoate (122 mg, 0.153 mmol, 61.7% yield) as a white solid. LCMS (M+H)=787.3; Retention time (0.05% TFA)=2.33 min. ¹H NMR (400 MHz, CDCl₃) δ 8.04 (s, 1H), 7.31-7.27 (m, 2H), 7.22-7.19 (m, 2H), 7.13 (dt, J=7.2, 0.8 Hz, 1H), 6.43 (t, J=6.8 Hz, 1H), 5.87 (brs, 2H), 5.65 (dd, J=7.2, 4.4 Hz, 1H), 4.43 (dd, J=11.2, 6.0 Hz, 1H), 4.35 (dd, J=10.8, 6.0 Hz, 1H), 4.11-4.04 (m, 2H), 3.99 (dd, J=10.8, 6.0 Hz, 1H), 3.78 (dd, J=11.2, 9.2 Hz, 1H), 2.73 (quint, J=7.2 Hz, 1H), 2.66-2.60 (m, 2H), 2.40 (t, J=7.6 Hz, 2H), 1.70-1.65 (m, 2H), 1.50 (quint, J=6.4 Hz, 1H), 1.39 (d, J=7.2 Hz, 3H), 1.35-1.25 (m, 20H), 0.91-0.85 (m, 9H).

### Example 56

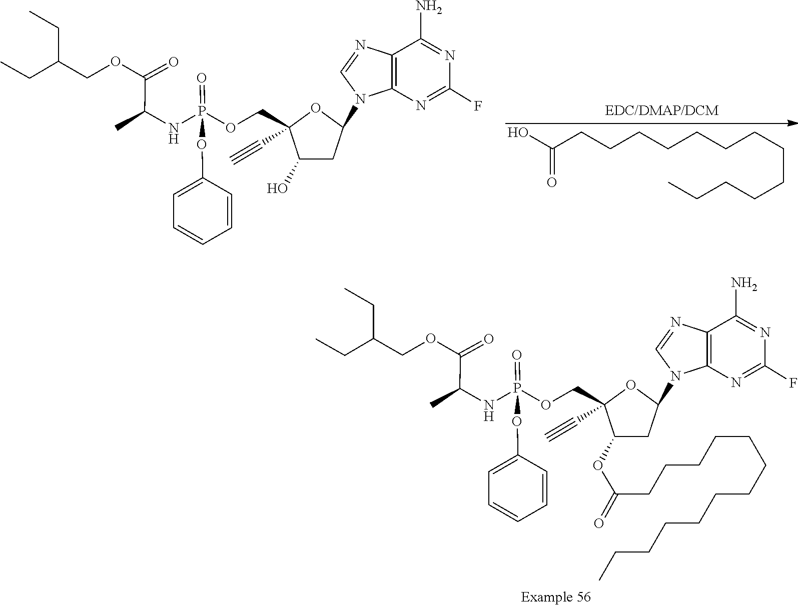 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCCCCCCCCCCCC(=O)O ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCCCCCC]

### (2R,3S,5R)-5-(6-Amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl tetradecanoate

To a solution of tetradecanoic acid (73.7 mg, 0.323 mmol) in DCM (6 mL) was added N,N-dimethylpyridin-4-amine (30.3 mg, 0.248 mmol) and 3-(((ethylimino)methylene)amino)-N,N-dimethylpropan-1-amine hydrochloride (95 mg, 0.496 mmol) and the resulting mixture was stirred for 0.5 h at an ice-water bath temperature. Then, 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.248 mmol) was added and the resulting mixture was stirred at 25° C. for 16 h. The LCMS showed the reaction was completed. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl tetradecanoate (117 mg, 0.137 mmol, 55.1% yield) as a white solid. LCMS (M+H)=815.4; Retention time (0.05% TFA)=2.55 min. ¹H NMR (400 MHz, CDCl₃) δ 8.04 (s, 1H), 7.31-7.27 (m, 2H), 7.22-7.19 (m, 2H), 7.13 (t, J=7.2 Hz, 1H), 6.43 (t, J=6.8 Hz, 1H), 5.91 (brs, 2H), 5.65 (dd, J=7.2, 4.4 Hz, 1H), 4.43 (dd, J=11.2, 6.4 Hz, 1H), 4.35 (dd, J=11.2, 6.0 Hz, 1H), 4.11-4.04 (m, 2H), 3.99 (dd, J=10.8, 6.0 Hz, 1H), 3.79 (dd, J=11.2, 9.6 Hz, 1H), 2.72 (quint, J=6.8 Hz, 1H), 2.66-2.59 (m, 2H), 2.40 (t, J=7.6 Hz, 2H), 1.70-1.67 (m, 2H), 1.50 (quint, J=6.4 Hz, 1H), 1.39 (d, J=6.8 Hz, 3H), 1.34-1.25 (m, 24H), 0.91-0.85 (m, 9H).

### Example 57

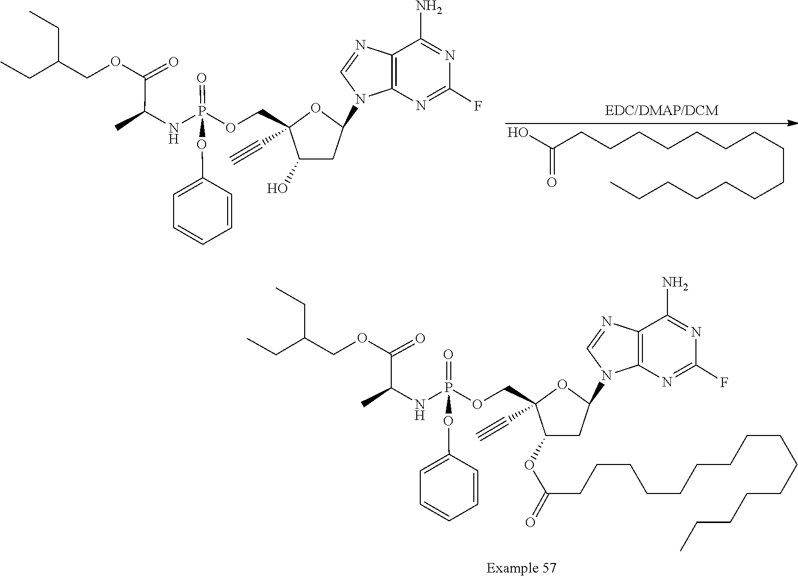 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCCCCCCCCCCCCCC(=O)O ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCCCCCCCC]

### (2R,3S,5R)-5-(6-Amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl palmitate

To a solution of palmitic acid (83 mg, 0.323 mmol) in DCM (6 mL) was added N,N-dimethylpyridin-4-amine (30.3 mg, 0.248 mmol) and 3-(((ethylimino)methylene)amino)-N,N-dimethylpropan-1-amine hydrochloride (95 mg, 0.496 mmol) and the resulting mixture was stirred for 0.5 h at an ice-water bath temperature. Then, 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.248 mmol) was added and the resulting mixture was stirred at 20° C. for 16 h. The LCMS showed the reaction was completed. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl palmitate (116 mg, 0.137 mmol, 55.1% yield) as a white solid. LCMS (M+H)=843.1; Retention time (0.1% TFA)=3.30 min. ¹H NMR (400 MHz, CDCl₃) δ 8.05 (s, 1H), 7.31-7.27 (m, 2H), 7.23-7.18 (m, 2H), 7.13 (t, J=7.2 Hz, 1H), 6.43 (t, J=6.8 Hz, 1H), 6.00 (brs, 2H), 5.65 (dd, J=6.8, 4.4 Hz, 1H), 4.43 (dd, J=11.2, 6.4 Hz, 1H), 4.35 (dd, J=10.8, 6.0 Hz, 1H), 4.11-4.04 (m, 2H), 3.99 (dd, J=10.8, 6.0 Hz, 1H), 3.84 (dd, J=10.8, 9.2 Hz, 1H), 2.72 (quint, J=6.8 Hz, 1H), 2.66-2.59 (m, 2H), 2.40 (t, J=7.6 Hz, 2H), 1.70-1.62 (m, 2H), 1.50 (quint, J=6.4 Hz, 1H), 1.39 (d, J=7.2 Hz, 3H), 1.34-1.23 (m, 28H), 0.90-0.84 (m, 9H).

### Example 58

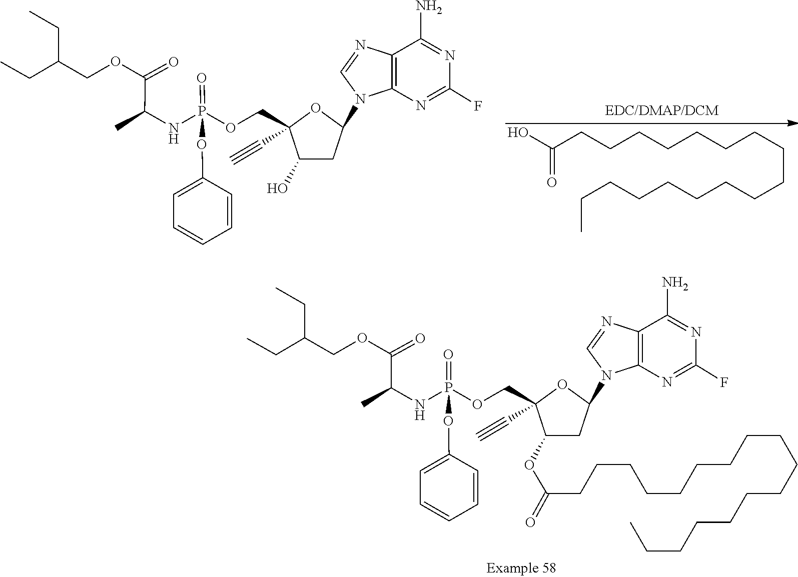 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCCCCCCCCCCCCCCCC(=O)O ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCCCCCCCCCC]

### (2R,3S,5R)-5-(6-Amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl stearate

To a solution of stearic acid (92 mg, 0.323 mmol) in DCM (6 mL) was added N,N-dimethylpyridin-4-amine (30.3 mg, 0.248 mmol) and 3-(((ethylimino)methylene)amino)-N,N-dimethylpropan-1-amine hydrochloride (95 mg, 0.496 mmol) and the resulting mixture was stirred for 0.5 h at an ice-water bath temperature. Then, 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.248 mmol) was added and the resulting mixture was stirred at 20° C. for 16 h. The LCMS showed the reaction was completed. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl stearate (151 mg, 0.173 mmol, 69.7% yield) as a white solid. LCMS (M+H)=871.3; Retention time (0.1% TFA)=3.90 min. ¹H NMR (400 MHz, CDCl₃) δ 8.04 (s, 1H), 7.31-7.27 (m, 2H), 7.23-7.19 (m, 2H), 7.13 (t, J=6.8 Hz, 1H), 6.43 (t, J=6.8 Hz, 1H), 5.92 (brs, 2H), 5.65 (dd, J=7.2, 4.4 Hz, 1H), 4.43 (dd, J=11.2, 6.0 Hz, 1H), 4.35 (dd, J=10.8, 6.0 Hz, 1H), 4.11-4.04 (m, 2H), 3.99 (dd, J=10.8, 6.0 Hz, 1H), 3.80 (dd, J=11.2, 9.6 Hz, 1H), 2.72 (quint, J=6.8 Hz, 1H), 2.66-2.59 (m, 2H), 2.40 (t, J=7.6 Hz, 2H), 1.66-1.62 (m, 2H), 1.50 (quint, J=6.4 Hz, 1H), 1.39 (d, J=7.2 Hz, 3H), 1.35-1.20 (m, 32H), 0.90-0.85 (m, 9H).

### Example 59

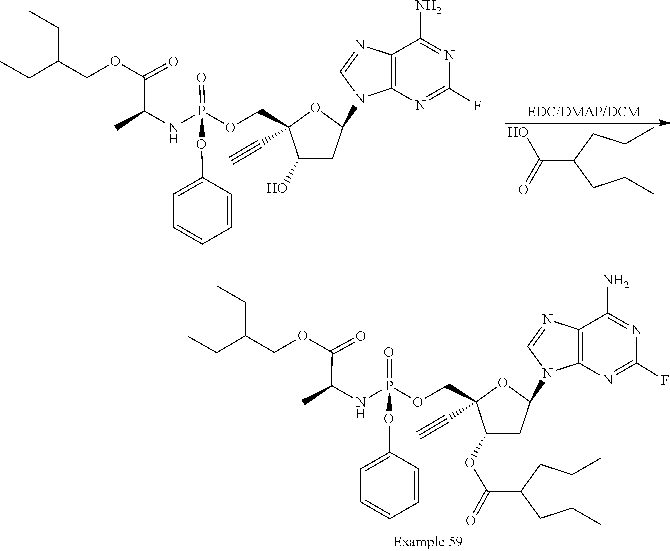

### (2R,3S,5R)-5-(6-Amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl 2-propylpentanoate

To a solution of 2-propylpentanoic acid (42.9 mg, 0.298 mmol) in DCM (6 mL) was added N,N-dimethylpyridin-4-amine (30.3 mg, 0.248 mmol) and 3-(((ethylimino)methylene)amino)-N,N-dimethylpropan-1-amine hydrochloride (95 mg, 0.496 mmol) and the resulting mixture was stirred for 0.5 h at an ice-water bath temperature. Then, 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.248 mmol) was added and the resulting mixture was stirred at 25° C. for 16 h. The LCMS showed the reaction was completed. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-((((S)-(((S)-1-(2-ethylbutoxy)-1-oxopropan-2-yl)amino)(phenoxy)phosphoryl)oxy)methyl)-2-ethynyltetrahydrofuran-3-yl 2-propylpentanoate (105 mg, 0.137 mmol, 55.3% yield) as a white solid. LCMS (M+H)=731.2; Retention time (0.05% TFA)=1.99 min. ¹H NMR (400 MHz, CDCl₃) δ 8.03 (s, 1H), 7.31-7.27 (m, 2H), 7.22-7.18 (m, 2H), 7.13 (dt, J=7.2, 0.8 Hz, 1H), 6.42 (t, J=6.8 Hz, 1H), 5.88 (brs, 2H), 5.66 (dd, J=7.2, 5.2 Hz, 1H), 4.44 (dd, J=11.2, 6.0 Hz, 1H), 4.33 (dd, J=11.2, 6.0 Hz, 1H), 4.11-4.05 (m, 2H), 3.99 (dd, J=10.8, 6.0 Hz, 1H), 3.81 (dd, J=11.2, 9.6 Hz, 1H), 2.79 (quint, J=6.8 Hz, 1H), 2.68-2.61 (m, 2H), 2.51-2.43 (m, 1H), 1.65-1.60 (m, 2H), 1.56-1.45 (m, 3H), 1.39 (d, J=6.8 Hz, 3H), 1.36-1.30 (m, 8H), 0.91 (dt, J=7.2, 1.2 Hz, 6H), 0.87 (t, J=7.6, Hz, 6H).

### Example 60

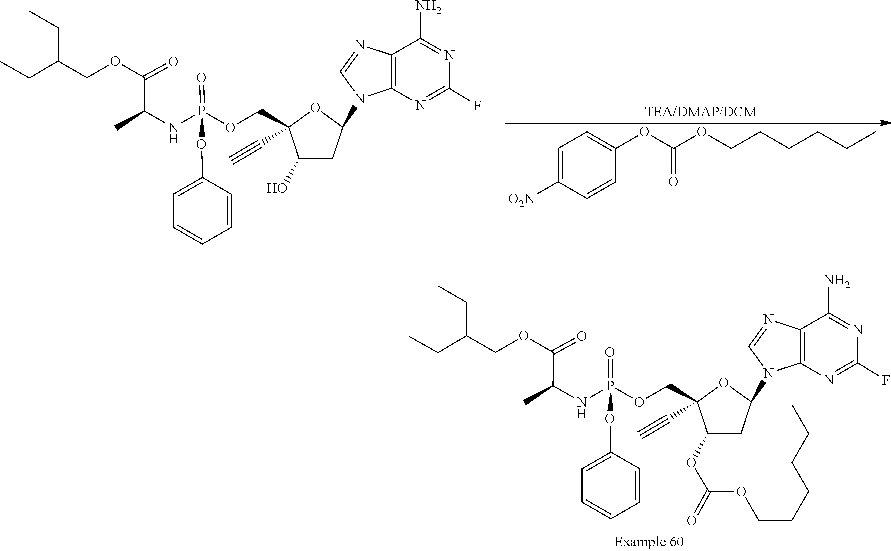 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCCCCOC(=O)Oc1ccc([N+](=O)[O-])cc1 ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCC]

### 2-Ethylbutyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-alaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.248 mmol) in DCM (10 mL) was treated with TEA (0.104 mL, 0.744 mmol), DMAP (30.3 mg, 0.248 mmol) followed by hexyl (4-nitrophenyl) carbonate (265 mg, 0.992 mmol) and the resulting mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford the desired Product 2-ethylbutyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-alaninate (100 mg, 0.136 mmol, 55.0% yield) as white solid. LCMS (M+H)=733.7; Retention time (0.1% TFA)=1.94 min. ¹H NMR (400 MHz, CDCl₃) δ 7.94 (s, 1H), 7.26-7.20 (m, 2H), 7.15-7.00 (m, 3H), 6.35 (t, J=6.7 Hz, 1H), 6.11-5.84 (m, 2H), 5.50 (dd, J=7.0, 4.4 Hz, 1H), 4.35 (ddd, J=33.6, 11.1, 6.1 Hz, 2H), 4.12 (td, J=6.7, 2.4 Hz, 2H), 4.04-3.90 (m, 2H), 3.87-3.73 (m, 1H), 2.77 (dd, J=14.0, 7.0 Hz, 1H), 2.70-2.56 (m, 2H), 1.75-1.54 (m, 3H), 1.42 (dd, J=12.5, 6.2 Hz, 1H), 1.32-1.18 (m, 10H), 0.81 (dt, J=10.6, 7.1 Hz, 9H).

### Example 61

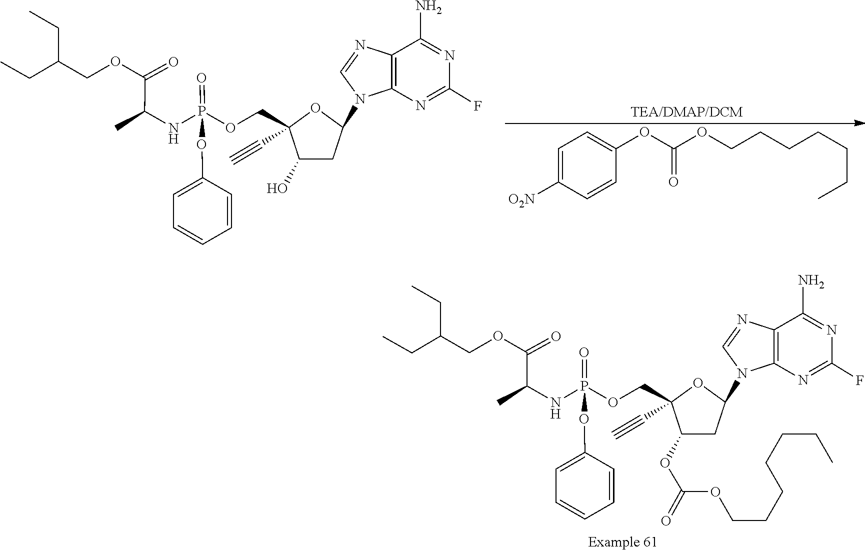 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCCCCCOC(=O)Oc1ccc([N+](=O)[O-])cc1 ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCC]

### 2-Ethylbutyl ((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)-L-alaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (150 mg, 0.248 mmol) in DCM (10 mL) was treated with TEA (0.104 mL, 0.744 mmol), DMAP (30.3 mg, 0.248 mmol) followed by heptyl (4-nitrophenyl) carbonate (279 mg, 0.992 mmol) and the mixture was stirred at RT for 2 days. LCMS showed the reaction was completed. Added water (20 mL), then the mixture was extracted with DCM (20 mL×3). The combined organic phases were washed with brine (80 mL), dried over Na₂SO₄ and concentrated in vacuum and the residue was purified by silica gel column chromatography (MeOH/DCM=0:100 to 1:20) to afford 2-ethylbutyl ((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)-L-alaninate (106 mg, 0.141 mmol, 56.6% yield) as white solid. LCMS (M+H)=747.7; Retention time (0.1% TFA)=1.99 min. ¹H NMR (400 MHz, CDCl₃) δ 7.94 (s, 1H), 7.26-7.20 (m, 2H), 7.15-7.00 (m, 3H), 6.35 (t, J=6.7 Hz, 1H), 6.11-5.84 (m, 2H), 5.50 (dd, J=7.0, 4.4 Hz, 1H), 4.35 (ddd, J=33.6, 11.1, 6.1 Hz, 2H), 4.12 (td, J=6.7, 2.4 Hz, 2H), 4.04-3.90 (m, 2H), 3.87-3.73 (m, 1H), 2.77 (dd, J=14.0, 7.0 Hz, 1H), 2.70-2.56 (m, 2H), 1.75-1.54 (m, 3H), 1.42 (dd, J=12.5, 6.2 Hz, 1H), 1.32-1.18 (m, 12H), 0.81 (dt, J=10.6, 7.1 Hz, 9H).

### Example 62

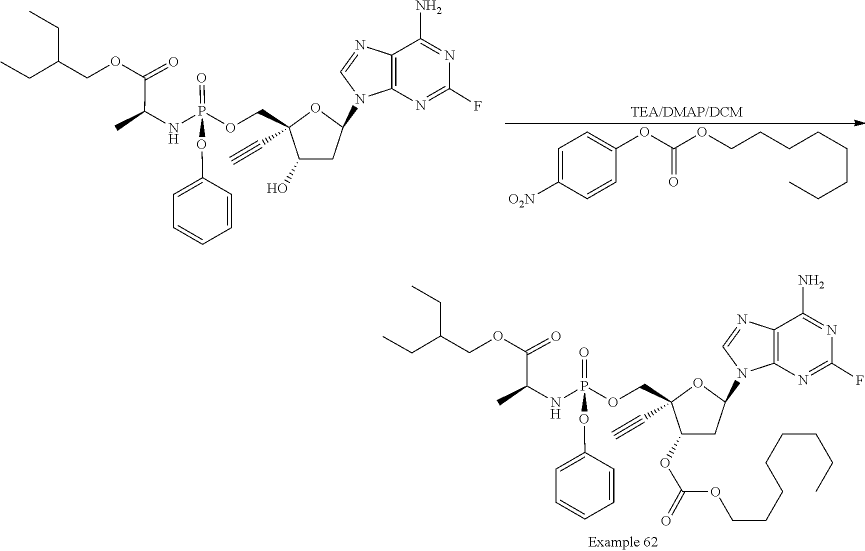 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCCCCCCOC(=O)Oc1ccc([N+](=O)[O-])cc1 ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCCC]

### 2-Ethylbutyl ((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)-L-alaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.331 mmol) in DCM (10 mL) was treated with TEA (0.138 mL, 0.992 mmol), DMAP (40.4 mg, 0.331 mmol) followed by 4-nitrophenyl octyl carbonate (391 mg, 1.323 mmol) and the mixture was stirred at RT for 2 days. LCMS showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford 2-ethylbutyl ((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)-L-alaninate (180 mg, 0.235 mmol, 71.2% yield) as white solid. LCMS (M+H)=761.7; Retention time (0.1% TFA)=2.04 min. ¹H NMR (400 MHz, CDCl₃) δ 7.94 (s, 1H), 7.26-7.20 (m, 2H), 7.15-7.00 (m, 3H), 6.35 (t, J=6.7 Hz, 1H), 6.11-5.84 (m, 2H), 5.50 (dd, J=7.0, 4.4 Hz, 1H), 4.35 (ddd, J=33.6, 11.1, 6.1 Hz, 2H), 4.12 (td, J=6.7, 2.4 Hz, 2H), 4.04-3.90 (m, 2H), 3.87-3.73 (m, 1H), 2.77 (dd, J=14.0, 7.0 Hz, 1H), 2.70-2.56 (m, 2H), 1.75-1.54 (m, 3H), 1.42 (dd, J=12.5, 6.2 Hz, 1H), 1.32-1.18 (m, 14H), 0.81 (dt, J=10.6, 7.1 Hz, 9H).

### Example 63

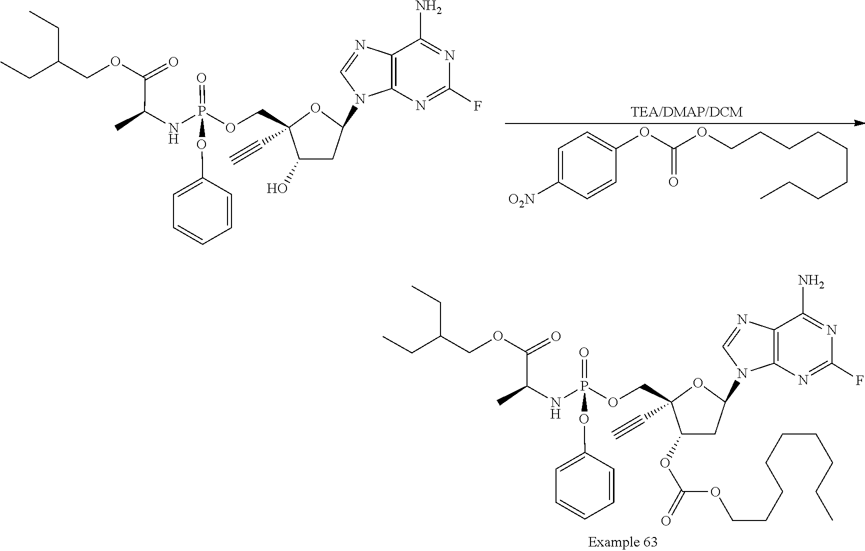 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCCCCCCCOC(=O)Oc1ccc([N+](=O)[O-])cc1 ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCCCC]

### 2-Ethylbutyl ((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)-L-alaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.331 mmol) and DMAP (40.4 mg, 0.331 mmol) in DCM (6 mL) was treated with TEA (0.138 mL, 0.992 mmol) followed by 4-nitrophenyl nonyl carbonate (409 mg, 1.323 mmol) and the mixture was stirred at 20° C. for 3 days. LCMS showed the reaction was completed. The reaction mixture was concentrated and the residue was purified by flash column chromatography (40 g, 0-4% MeOH in DCM) to give 2-ethylbutyl ((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)-L-alaninate (132 mg, 0.166 mmol, 50.0% yield) as a white solid. LCMS (M+H)=775.3; Retention time (0.05% TFA)=2.11 min. ¹H NMR (400 MHz, CDCl₃) δ 8.06 (s, 1H), 7.30-7.25 (m, 2H), 7.22-7.18 (m, 2H), 7.12 (t, J=7.2, 0.8 Hz, 1H), 6.42 (t, J=6.8 Hz, 1H), 6.22 (brs, 2H), 5.56 (dd, J=6.8, 4.4 Hz, 1H), 4.46 (dd, J=11.2, 6.4 Hz, 1H), 4.38 (dd, J=11.2, 6.0 Hz, 1H), 4.19 (dt, J=6.8, 2.0 Hz, 2H), 4.11-4.04 (m, 2H), 3.99 (dd, J=11.2, 6.0 Hz, 1H), 3.94 (dd, J=11.2, 9.6 Hz, 1H), 2.83 (quint, J=7.2 Hz, 1H), 2.72 (ddd, J=14.0, 6.4, 4.4 Hz, 1H), 2.68 (s, 1H), 1.70 (quint, J=6.8 Hz, 2H), 1.50 (quint, J=6.4 Hz, 1H), 1.39 (dd, J=7.2 Hz, 3H), 1.36-1.25 (m, 16H), 0.91-0.84 (m, 9H).

### Example 64

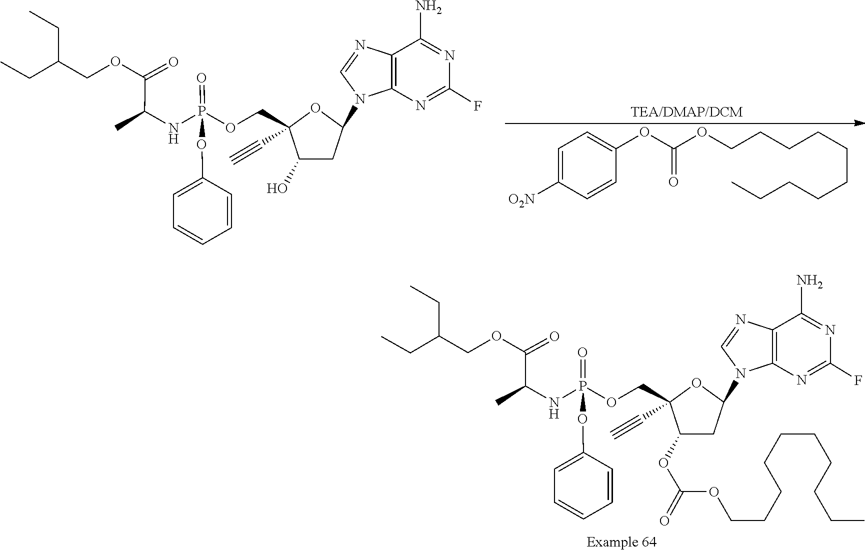 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCCCCCCCCOC(=O)Oc1ccc([N+](=O)[O-])cc1 ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCCCCC]

### 2-Ethylbutyl ((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)-L-alaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.331 mmol) and DMAP (40.4 mg, 0.331 mmol) in DCM (6 mL) was treated with TEA (0.138 mL, 0.992 mmol) followed by decyl (4-nitrophenyl) carbonate (428 mg, 1.323 mmol) and the mixture was stirred at 20° C. for 3 days. LCMS showed the reaction was completed. The reaction mixture was concentrated and the residue was purified by flash column chromatography (40 g, 0-4% MeOH in DCM) to give 2-ethylbutyl ((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)-L-alaninate (109 mg, 0.134 mmol, 40.4% yield) as a white solid. LCMS (M+H)=789.3; Retention time (0.05% TFA)=2.18 min. ¹H NMR (400 MHz, CDCl₃) δ 8.01 (s, 1H), 7.30-7.25 (m, 2H), 7.22-7.18 (m, 2H), 7.13 (t, J=7.2, 0.8 Hz, 1H), 6.42 (t, J=6.8 Hz, 1H), 5.98 (brs, 2H), 5.56 (dd, J=6.8, 4.4 Hz, 1H), 4.46 (dd, J=11.2, 6.4 Hz, 1H), 4.38 (dd, J=11.2, 6.0 Hz, 1H), 4.19 (dt, J=6.8, 2.0 Hz, 2H), 4.11-4.04 (m, 2H), 3.99 (dd, J=10.8, 6.0 Hz, 1H), 3.84 (dd, J=11.2, 9.2 Hz, 1H), 2.85 (quint, J=7.2 Hz, 1H), 2.72 (ddd, J=14.0, 6.4, 4.4 Hz, 1H), 2.68 (s, 1H), 1.70 (quint, J=6.8 Hz, 2H), 1.50 (quint, J=6.4 Hz, 1H), 1.39 (dd, J=6.8 Hz, 3H), 1.35-1.25 (m, 18H), 0.91-0.85 (m, 9H).

### Example 65

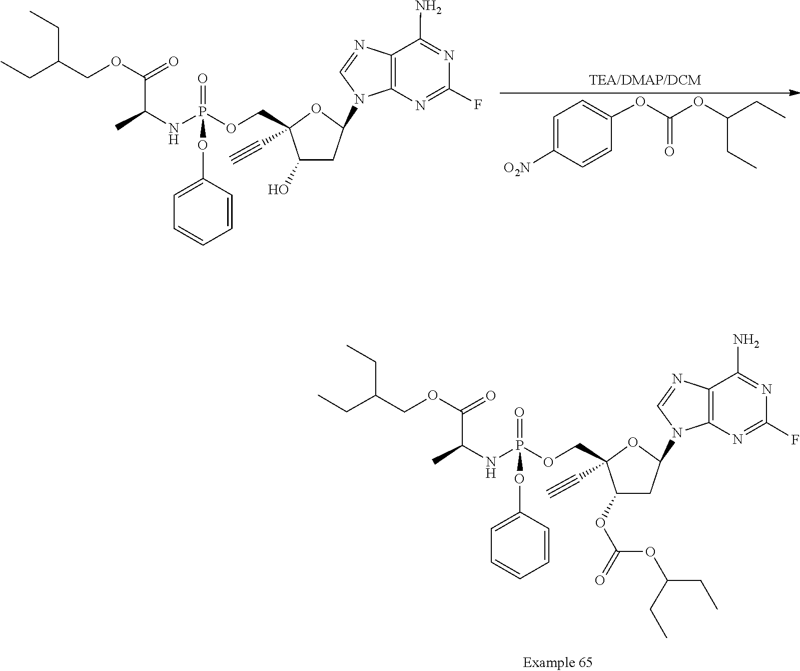 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCC(CC)OC(=O)Oc1ccc([N+](=O)[O-])cc1 ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OC(CC)CC]

### 2-Ethylbutyl ((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)-L-alaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.331 mmol) and DMAP (40.4 mg, 0.331 mmol) in DCM (6 mL) was treated with TEA (0.138 mL, 0.992 mmol) followed by 4-nitrophenyl pentan-3-yl carbonate (335 mg, 1.323 mmol) and the mixture was stirred at 25° C. for 4 days. LCMS showed the reaction was finished. The reaction mixture was concentrated and the residue was purified by reverse phase chromatography (SepaFlash@ C18 column, BOSTON, 40 g, 0-100% MeOH/10 mM aq.NH₄HCO₃) to give 2-ethylbutyl ((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)-L-alaninate (100 mg, 0.134 mmol, 40.5% yield) as a white solid. LCMS (M+H)=719.2; Retention time (0.05% TFA)=1.87 min. ¹H NMR (400 MHz, CDCl₃) δ 8.02 (s, 1H), 7.30-7.25 (m, 2H), 7.22-7.18 (m, 2H), 7.12 (dt, J=7.2, 0.8 Hz, 1H), 6.43 (t, J=7.2 Hz, 1H), 6.08 (brs, 2H), 5.56 (dd, J=6.8, 4.0 Hz, 1H), 4.63 (quint, J=6.0 Hz, 1H), 4.47 (dd, J=11.2, 6.4 Hz, 1H), 4.39 (dd, J=10.8, 6.0 Hz, 1H), 4.12-4.05 (m, 2H), 3.99 (dd, J=10.8, 5.6 Hz, 1H), 3.89 (dd, J=11.2, 9.6 Hz, 1H), 2.83 (quint, J=7.2 Hz, 1H), 2.71 (ddd, J=14.0, 6.0, 4.0 Hz, 1H), 2.67 (s, 1H), 1.66 (quint, J=7.2 Hz, 4H), 1.50 (quint, J=6.0 Hz, 1H), 1.39 (d, J=7.2 Hz, 3H), 1.35-1.29 (m, 4H), 0.94 (dt, J=7.6, 1.2 Hz, 6H), 0.87 (t, J=7.6, Hz, 6H).

### Example 66

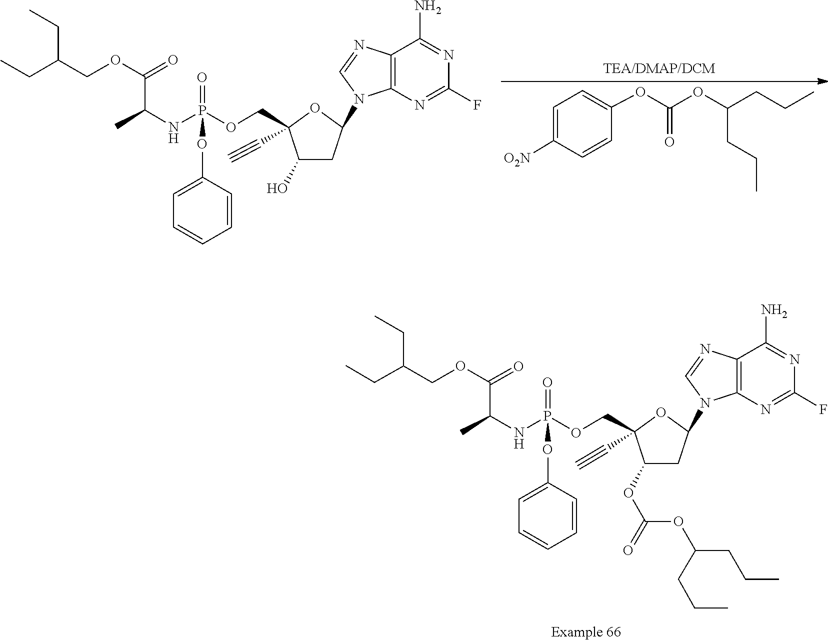 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCC(CCC)OC(=O)Oc1ccc([N+](=O)[O-])cc1 ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OC(CCC)CCC]

### 2-Ethylbutyl ((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)-L-alaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.331 mmol) and N,N-dimethylpyridin-4-amine (40.4 mg, 0.331 mmol) in DCM (6 mL) was treated with TEA (0.138 mL, 0.992 mmol) followed by heptan-4-yl (4-nitrophenyl) carbonate (372 mg, 1.323 mmol) and the mixture was stirred at 25° C. for 4 days. LCMS showed the reaction was finished. The reaction mixture was concentrated and the residue was purified by silica gel column chromatography (40 g, 3% MeOH in DCM) to give 2-ethylbutyl ((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)-L-alaninate (99 mg, 0.133 mmol, 40.1% yield) as a white solid. LCMS (M+H)=747.3; Retention time (0.05% TFA)=1.97 min. ¹H NMR (400 MHz, CDCl₃) δ 8.02 (s, 1H), 7.30-7.27 (m, 2H), 7.22-7.18 (m, 2H), 7.12 (dt, J=7.2, 0.8 Hz, 1H), 6.42 (t, J=6.8 Hz, 1H), 5.91 (brs, 2H), 5.55 (dd, J=7.2, 4.0 Hz, 1H), 4.81-4.74 (m, 1H), 4.47 (dd, J=11.2, 6.4 Hz, 1H), 4.39 (dd, J=11.2, 6.4 Hz, 1H), 4.12-4.05 (m, 2H), 3.99 (dd, J=10.8, 5.6 Hz, 1H), 3.80 (dd, J=11.2, 9.6 Hz, 1H), 2.83 (quint, J=7.2 Hz, 1H), 2.71 (ddd, J=14.0, 6.0, 4.0 Hz, 1H), 2.66 (s, 1H), 1.68-1.42 (m, 7H), 1.39 (d, J=7.2 Hz, 3H), 1.36-1.29 (m, 6H), 0.93 (dt, J=12.4, 7.6 Hz, 6H), 0.87 (t, J=7.6, Hz, 6H).

### Example 67

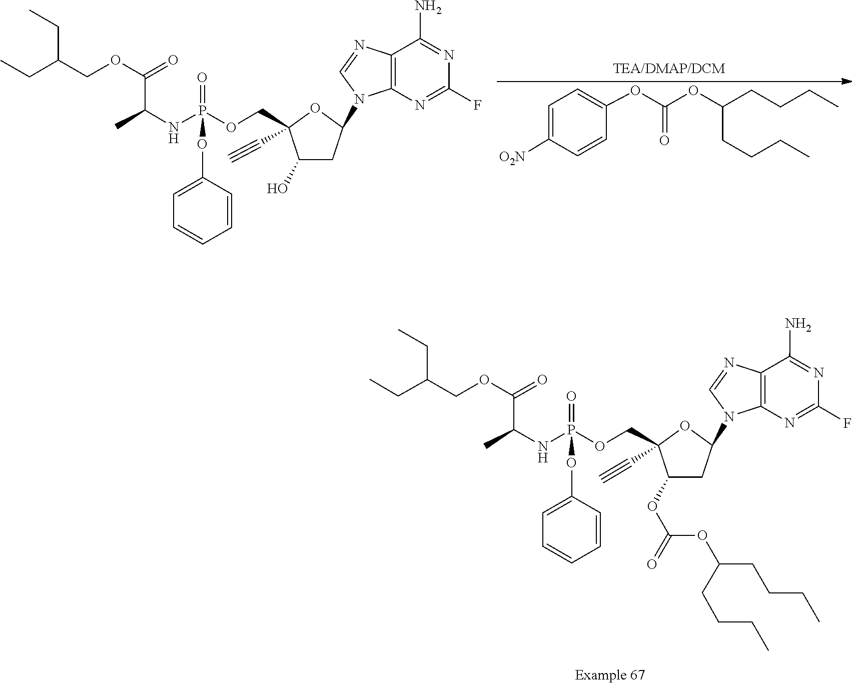 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1O ; CCCCC(CCCC)OC(=O)Oc1ccc([N+](=O)[O-])cc1 ; C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OC(CCCC)CCCC]

### 2-Ethylbutyl ((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)-L-alaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (200 mg, 0.331 mmol and N,N-dimethylpyridin-4-amine (40.4 mg, 0.331 mmol) in DCM (6 mL) was treated with TEA (0.138 mL, 0.992 mmol) followed by 4-nitrophenyl nonan-5-yl carbonate (409 mg, 1.323 mmol) and the mixture was stirred at 25° C. for 3 days. LCMS showed the reaction was finished. The reaction mixture was concentrated and the residue was purified by silica gel column chromatography (40 g, 0-3% MeOH in DCM) to give 2-ethylbutyl ((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)-L-alaninate (142 mg, 0.183 mmol, 55.4% yield) as a white solid. LCMS (M+H)=775.3; Retention time (0.05% TFA)=2.10 min. ¹H NMR (400 MHz, CDCl₃) δ 8.02 (s, 1H), 7.31-7.27 (m, 2H), 7.22-7.18 (m, 2H), 7.12 (dt, J=7.2, 0.8 Hz, 1H), 6.43 (t, J=6.8 Hz, 1H), 5.91 (brs, 2H), 5.55 (dd, J=7.2, 4.0 Hz, 1H), 4.77-4.70 (m, 1H), 4.46 (dd, J=10.8, 6.0 Hz, 1H), 4.39 (dd, J=10.8, 6.0 Hz, 1H), 4.12-4.05 (m, 2H), 3.99 (dd, J=10.8, 5.6 Hz, 1H), 3.80 (dd, J=10.8, 9.2 Hz, 1H), 2.83 (quint, J=7.2 Hz, 1H), 2.71 (ddd, J=14.0, 6.0, 4.0 Hz, 1H), 2.65 (s, 1H), 1.65-1.56 (m, 4H), 1.50 (quint, J=6.4 Hz, 1H), 1.39 (d, J=6.8 Hz, 3H), 1.36-1.29 (m, 12H), 0.92-0.85 (m, 12H).

### Example 68

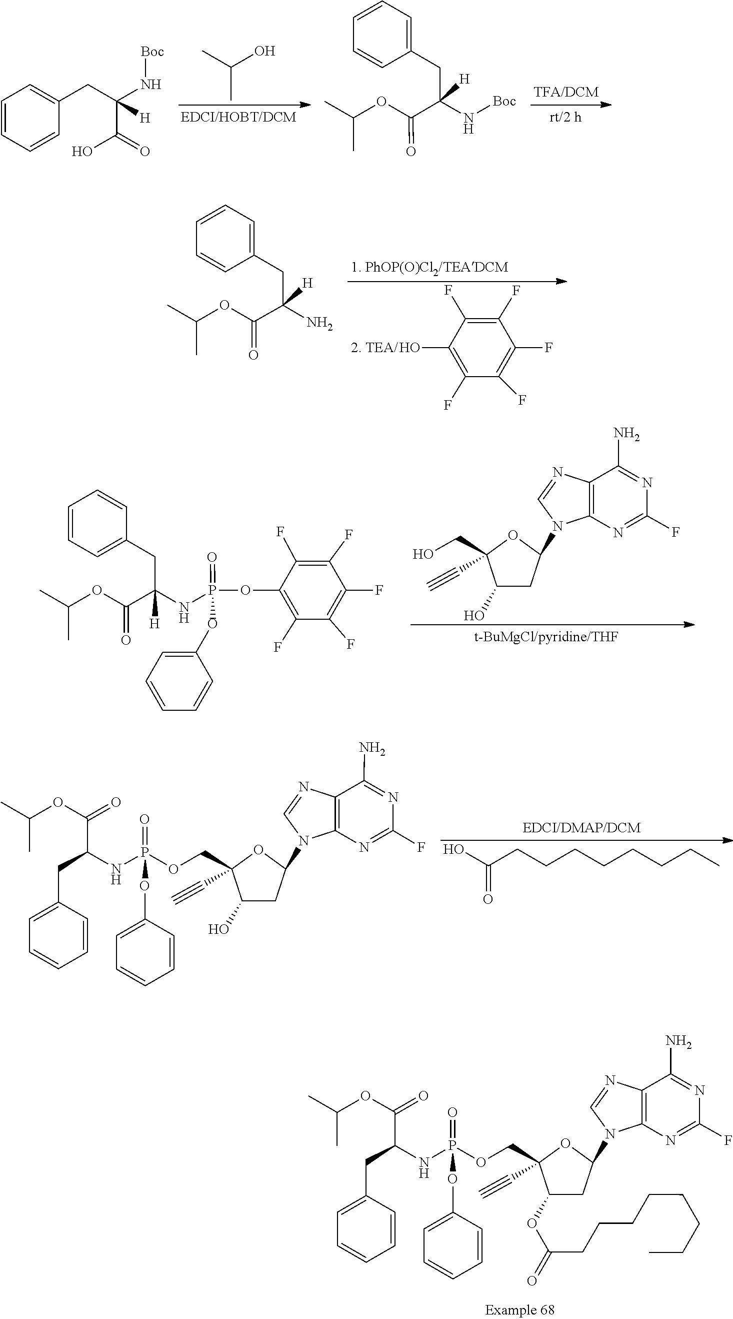

### Step 1: Isopropyl (tert-butoxycarbonyl)-L-phenylalaninate

A mixture of propan-2-ol (13.59 g, 226 mmol)), HOBt (8.66 g, 56.5 mmol), EDC (32.5 g, 170 mmol) in DCM (250 mL) was added DIEA (59.2 mL, 339 mmol) at −10° C. After stirring for 30 min, (tert-butoxycarbonyl)-L-phenylalanine (30 g, 113 mmol) in DCM (50 mL) was added and stirred at 25° C. for 16 h. TLC showed the presence of new compound. The reaction was diluted with water (500 mL) and extracted with DCM (250 mL×3). The combined organic layers were washed with brine (150 mL), dried with Na₂SO₄, filtered and concentrated in vacuum to give the residue, which was purified by flash column chromatography (pet. ether/EtOAc=100/0 to 20/1) to give isopropyl (tert-butoxycarbonyl)-L-phenylalaninate (23 g, 74.8 mmol, 66.2% yield) as colorless oil. ¹H NMR (400 MHz, CDCl₃) δ 7.36-7.19 (m, 3H), 7.15 (d, J=6.9 Hz, 2H), 5.00 (s, 2H), 4.63-4.41 (m, 1H), 3.17-2.95 (m, 2H), 1.42 (s, 9H), 1.20 (dd, J=11.9, 6.3 Hz, 6H).

### Step 2: Isopropyl L-phenylalaninate

To a solution of isopropyl (tert-butoxycarbonyl)-L-phenylalaninate (23 g, 74.8 mmol) in DCM (300 mL) was added TFA (57.6 mL, 748 mmol) at −10° C. under N₂. The reaction was stirred for 16 h at 25° C. under N₂. TLC showed the reaction was completed. The reaction was concentrated in vacuum to give the residue which was diluted with water (250 mL) and pH was adjusted to 7 with a solution of NaHCO₃and extracted with EtOAc (250 mL×3). The combined organic layers were washed with brine (150 mL), dried over sodium sulfate and evaporated. The residue was purified by flash column chromatography (DCM/MeOH=100/0 to 20/1) to give isopropyl L-phenylalaninate (10 g, 48.2 mmol, 64.5% yield) as yellow oil. LCMS (M+H)=208.0; Retention time (0.1% TFA)=1.169 min.

### Step 3: Isopropyl ((S)-(perfluorophenoxy)(phenoxy)phosphoryl)-L-phenylalaninate

To a solution of isopropyl L-phenylalaninate (10 g, 48.2 mmol) in anhydrous DCM (100 mL) was added dropwise triethylamine (7.38 mL, 53.1 mmol) at −70° C. over 15 min. To this mixture was added a solution of phenyl phosphorodichloridate (10.08 g, 47.8 mmol) in anhydrous DCM (50 mL) over 1 h. The reaction mixture was stirred at this temperature for additional 30 min and then allowed to warm to 0° C. over 2 h and stirred for additional 1 h. To this mixture was added a solution of 2,3,4,5,6-pentafluorophenol (8.79 g, 47.8 mmol) and triethylamine (7.38 mL, 53.1 mmol) in DCM (30 mL) over 20 min. The crude mixture was allowed to stir at 0° C. for 4 h. LCMS showed the reaction was completed. The white solid (triethylaminehydrochloride) was filtered off and washed with dichloromethane (2×25 mL). The filtrate was concentrated under reduced pressure, the residue was triturated with TBME (200 mL), and the triethylaminehydrochloride salt was removed by filtration. The filter cake was washed with TBME (2×25 mL), and the combined filtrate was concentrated under reduced pressure to give 16 g crude product as a mixture of diastereomers. The mixture was triturated with 20% EtOAc in hexanes (100 mL) and collected by filtration to give isopropyl ((S)-(perfluorophenoxy)(phenoxy)phosphoryl)-L-phenylalaninate (6.0 g, 11.33 mmol, 23.49% yield) as a white solid (>98% de as determined by ³¹PNMR). ¹H NMR (400 MHz, CDCl₃) δ 7.35 (t, J=7.8 Hz, 2H), 7.25-7.15 (m, 6H), 7.12-7.03 (m, 2H), 4.96 (dt, J=12.5, 6.2 Hz, 1H), 4.39 (ddd, J=15.8, 10.0, 6.0 Hz, 1H), 3.79 (t, J=11.1 Hz, 1H), 3.14 (dd, J=13.7, 5.3 Hz, 1H), 3.04 (dd, J=13.7, 6.5 Hz, 1H), 1.16 (dd, J=16.8, 6.3 Hz, 6H). ³¹P NMR (162 MHz, DMSO-d₆) δ−1.54.

### Step 4: Isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a solution of (2R,3S,5R)-5-(6-amino-2-fluoro-9H-purin-9-yl)-2-ethynyl-2-(hydroxymethyl)tetrahydrofuran-3-ol (0.25 g, 0.852 mmol) in THF (40 mL) and pyridine (2.0 mL) was added dropwise tert-butylmagnesium chloride (1.79 mL, 1.79 mmol) at 0° C. Then, the reaction was stirred at 25° C. for 30 min. A solution of isopropyl ((S)-(perfluorophenoxy)(phenoxy)phosphoryl)-L-phenylalaninate (0.587 g, 1.108 mmol) in THF (20 mL) was added dropwise to the above solution at 0° C. and stirred at 0° C. for 4 h. TLC showed completion of reaction. The reaction was diluted with 2 M HCl (20 mL) and extracted with EtOAc (50 mL×3). The combined organic layers were washed with a solution of NaHCO₃ (20 mL), brine (50 mL), dried with Na₂SO₄, filtered and concentrated in vacuum to give the crude product. The crude product was purified by flash column chromatography (DCM/MeOH=100/0 to 10/1) to give isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (133 mg, 0.204 mmol, 23.94% yield) as white solid. LCMS (M+H)=639; Retention time (0.1% TFA)=1.577 min.

### Step 5: (2R,3S,5R)-5-(6-Amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl nonanoate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.235 mmol) in DCM (10 mL) was treated with EDC (90 mg, 0.470 mmol), DMAP (28.7 mg, 0.235 mmol) followed by nonanoic acid (44.6 mg, 0.282 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford (2R,3S,5R)-5-(6-amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl nonanoate (115 mg, 0.148 mmol, 62.9% yield) as white solid. LCMS LCMS (M+H)=778.7; Retention time (0.1% 10 mM NH4HCO3)=2.126 min. ¹H NMR (400 MHz, CDCl₃) δ 8.01 (s, 1H), 7.25-7.07 (m, 10H), 6.40 (t, J=6.7 Hz, 1H), 5.91 (s, 2H), 5.57 (dd, J=6.7, 4.2 Hz, 1H), 4.94 (dt, J=12.5, 6.2 Hz, 1H), 4.35-4.22 (m, 2H), 4.13 (dd, J=11.0, 5.7 Hz, 1H), 3.61 (t, J=10.7 Hz, 1H), 2.99 (d, J=6.4 Hz, 2H), 2.73-2.52 (m, 3H), 2.40 (t, J=7.6 Hz, 2H), 1.68-1.62 (m, 2H), 1.28 (s, 10H), 1.14 (dd, J=7.5, 6.4 Hz, 6H), 0.89 (t, J=6.8 Hz, 3H).

### Example 69

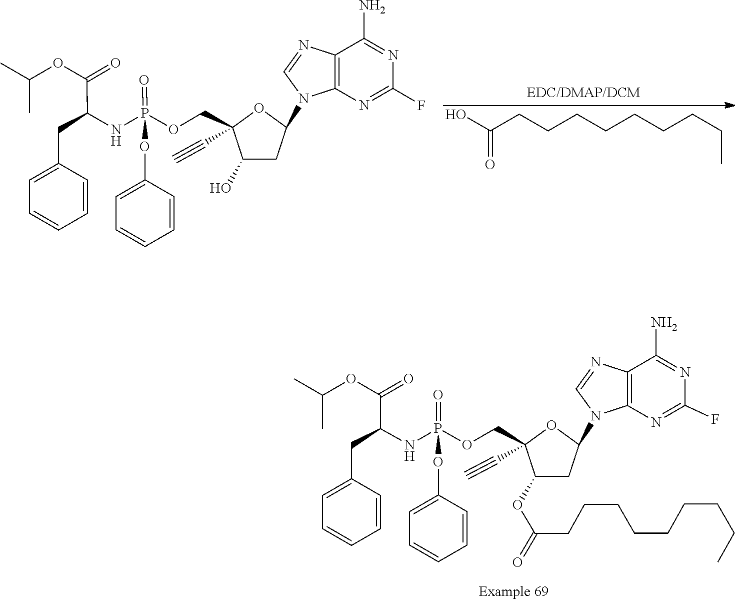

### (2R,3S,5R)-5-(6-Amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl decanoate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.235 mmol) in DCM (10 mL) was treated with EDC (90 mg, 0.470 mmol), DMAP (28.7 mg, 0.235 mmol) followed by decanoic acid (48.6 mg, 0.282 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford (2R,3S,5R)-5-(6-amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl decanoate (150 mg, 0.186 mmol, 79% yield) as white solid. LCMS (M+H)=792.7; Retention time (0.1% TFA)=2.089 min; ¹H NMR (400 MHz, CDCl₃) δ 8.00 (s, 1H), 7.26-7.07 (m, 10H), 6.40 (t, J=6.8 Hz, 1H), 6.04 (s, 2H), 5.58 (dd, J=6.7, 4.1 Hz, 1H), 4.94 (hept, J=6.3 Hz, 1H), 4.29 (ddd, J=18.5, 17.0, 13.0 Hz, 2H), 4.13 (dd, J=11.0, 5.7 Hz, 1H), 3.67 (t, J=10.7 Hz, 1H), 3.10-2.91 (m, 2H), 2.73-2.53 (m, 3H), 2.40 (t, J=7.6 Hz, 2H), 1.75-1.59 (m, 2H), 1.30 (d, J=18.5 Hz, 12H), 1.14 (dd, J=8.3, 6.3 Hz, 6H), 0.88 (t, J=6.8 Hz, 3H).

### Example 70

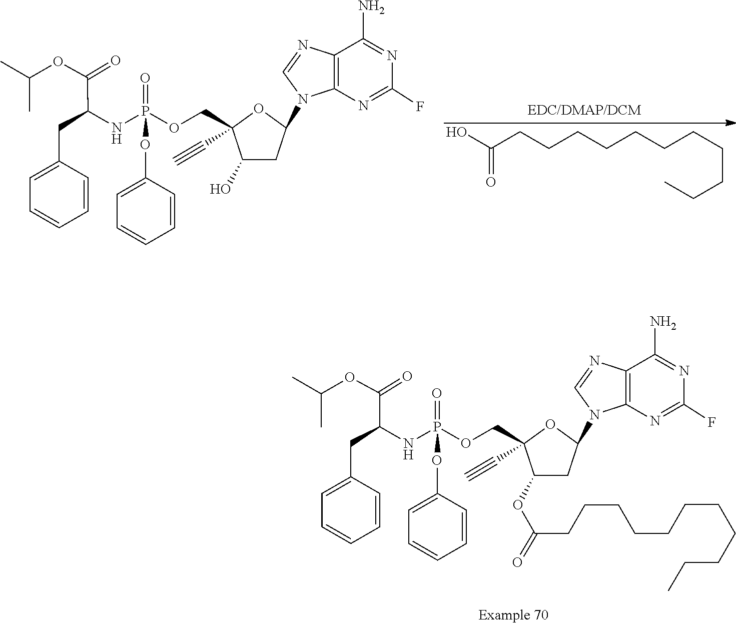 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1O ; CCCCCCCCCCCC(=O)O ; C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCC]

### (2R,3S,5R)-5-(6-amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl dodecanoate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.235 mmol) in DCM (10 mL) was treated with EDC (90 mg, 0.470 mmol), DMAP (28.7 mg, 0.235 mmol) followed by dodecanoic acid (56.5 mg, 0.282 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford (2R,3S,5R)-5-(6-amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl dodecanoate (105.2 mg, 0.128 mmol, 54.6% yield) as white solid. LCMS (M+H)=821.7; Retention time (0.1% TFA)=2.253 min. ¹H NMR:N78514-83-A1; ¹H NMR (400 MHz, CDCl₃) δ 8.00 (s, 1H), 7.26-7.08 (m, 10H), 6.40 (t, J=6.7 Hz, 1H), 5.94 (s, 2H), 5.57 (dd, J=6.7, 4.2 Hz, 1H), 5.01-4.78 (m, 1H), 4.28 (ddd, J=21.0, 10.7, 6.4 Hz, 2H), 4.13 (dd, J=11.0, 5.7 Hz, 1H), 3.63 (dd, J=20.6, 9.9 Hz, 1H), 2.99 (d, J=6.4 Hz, 2H), 2.74-2.53 (m, 3H), 2.47-2.31 (m, 2H), 1.64 (d, J=7.5 Hz, 2H), 1.29 (d, J=22.9 Hz, 16H), 1.14 (dd, J=8.0, 6.3 Hz, 6H), 0.88 (t, J=6.8 Hz, 3H).

### Example 71

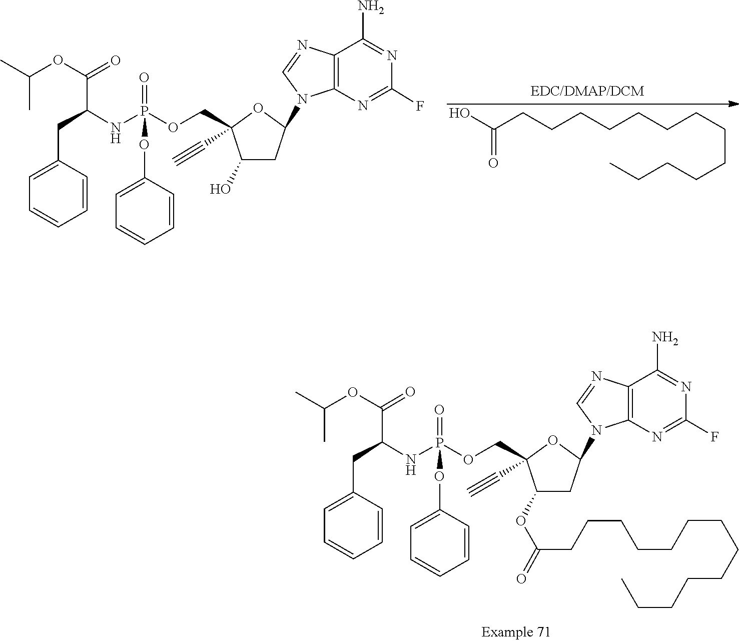

### (2R,3S,5R)-5-(6-Amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl tetradecanoate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.235 mmol) in DCM (10 mL) was treated with EDC (90 mg, 0.470 mmol), DMAP (28.7 mg, 0.235 mmol) followed by tetradecanoic acid (64.4 mg, 0.282 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford (2R,3S,5R)-5-(6-amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl tetradecanoate (110 mg, 0.130 mmol, 55.2% yield) as white solid. LCMS (M+H)=848.7; Retention time (0.1% TFA)=2.452 min. ¹H NMR (400 MHz, CDCl₃) δ 8.00 (s, 1H), 7.26-7.08 (m, 10H), 6.40 (t, J=6.7 Hz, 1H), 5.94 (s, 2H), 5.57 (dd, J=6.7, 4.2 Hz, 1H), 5.01-4.78 (m, 1H), 4.28 (ddd, J=21.0, 10.7, 6.4 Hz, 2H), 4.13 (dd, J=11.0, 5.7 Hz, 1H), 3.63 (dd, J=20.6, 9.9 Hz, 1H), 2.99 (d, J=6.4 Hz, 2H), 2.74-2.53 (m, 3H), 2.47-2.31 (m, 2H), 1.64 (d, J=7.5 Hz, 2H), 1.29 (d, J=22.9 Hz, 20H), 1.14 (dd, J=8.0, 6.3 Hz, 6H), 0.88 (t, J=6.8 Hz, 3H).

### Example 72

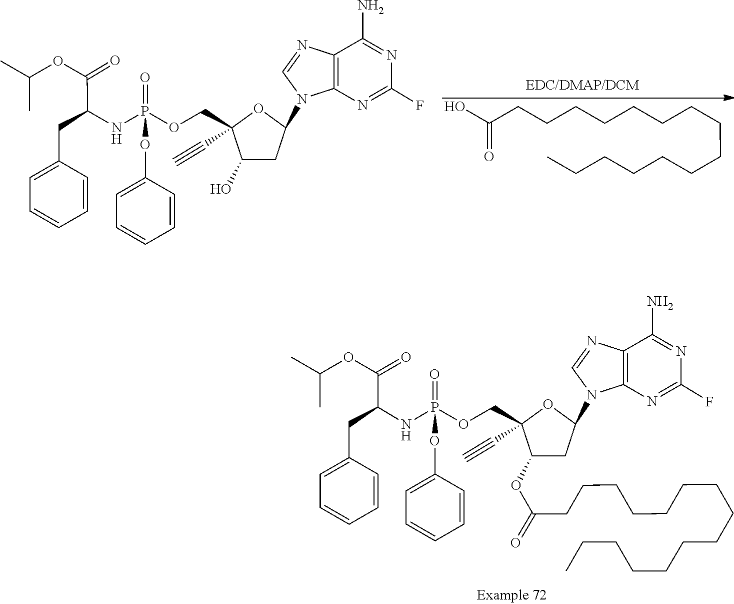

### Example 72

### (2R,3S,5R)-5-(6-Amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl palmitate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.235 mmol) in DCM (10 mL) was treated with EDC (90 mg, 0.470 mmol), DMAP (28.7 mg, 0.235 mmol) followed by palmitic acid (72.3 mg, 0.282 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford (2R,3S,5R)-5-(6-amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl palmitate (110.2 mg, 0.126 mmol, 53.5% yield) as white solid. LCMS (M+H)=876.7; Retention time (0.1% TFA)=2.72 min; purity (254 nm)=100%. ¹H NMR (400 MHz, CDCl₃) δ 8.01 (s, 1H), 7.26-7.07 (m, 10H), 6.40 (t, J=6.2 Hz, 1H), 6.31 (s, 2H), 5.57 (dd, J=6.6, 4.2 Hz, 1H), 4.93 (dq, J=12.4, 6.2 Hz, 1H), 4.34-4.18 (m, 2H), 4.14 (dd, J=11.1, 5.7 Hz, 1H), 3.80 (t, J=10.8 Hz, 1H), 3.09-2.91 (m, 2H), 2.67-2.53 (m, 3H), 2.40 (t, J=7.6 Hz, 2H), 1.72-1.60 (m, 2H), 1.29 (d, J=24.7 Hz, 24H), 1.14 (dd, J=10.2, 6.3 Hz, 6H), 0.88 (t, J=6.8 Hz, 3H).

### Example 73

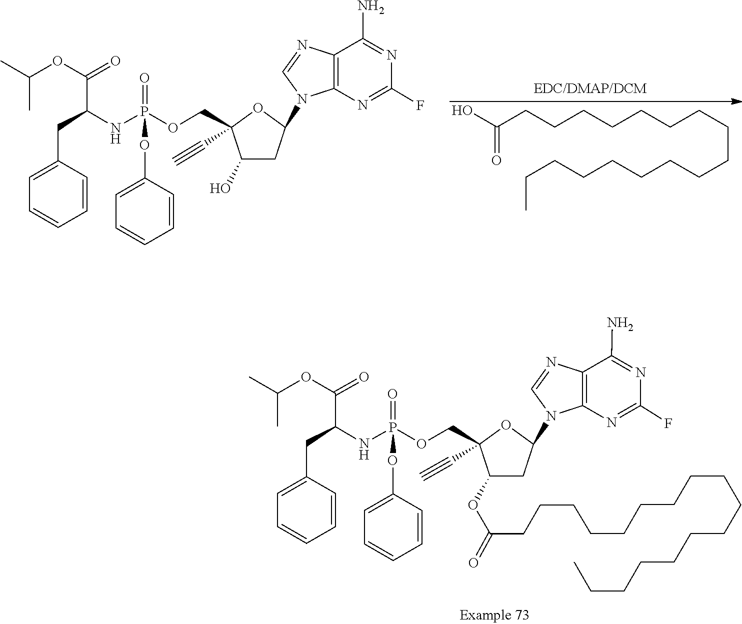 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1O ; CCCCCCCCCCCCCCCCCC(=O)O ; C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCCCCCCCC]

### (2R,3S,5R)-5-(6-Amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl stearate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.235 mmol) in DCM (10 mL) was treated with EDC (90 mg, 0.470 mmol), DMAP (28.7 mg, 0.235 mmol) followed by stearic acid (66.8 mg, 0.235 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford (2R,3S,5R)-5-(6-amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl stearate (130.0 mg, 0.144 mmol, 61.2% yield) as white solid. LCMS (M+H)=904.7; Retention time (0.1% TFA)=3.65 min. ¹H NMR (400 MHz, CDCl₃) δ 8.01 (s, 1H), 7.26-7.07 (m, 10H), 6.40 (t, J=6.2 Hz, 1H), 6.31 (s, 2H), 5.57 (dd, J=6.6, 4.2 Hz, 1H), 4.93 (dq, J=12.4, 6.2 Hz, 1H), 4.34-4.18 (m, 2H), 4.14 (dd, J=11.1, 5.7 Hz, 1H), 3.80 (t, J=10.8 Hz, 1H), 3.09-2.91 (m, 2H), 2.67-2.53 (m, 3H), 2.40 (t, J=7.6 Hz, 2H), 1.72-1.60 (m, 2H), 1.29 (d, J=24.7 Hz, 28H), 1.14 (dd, J=10.2, 6.3 Hz, 6H), 0.88 (t, J=6.8 Hz, 3H).

### Example 74

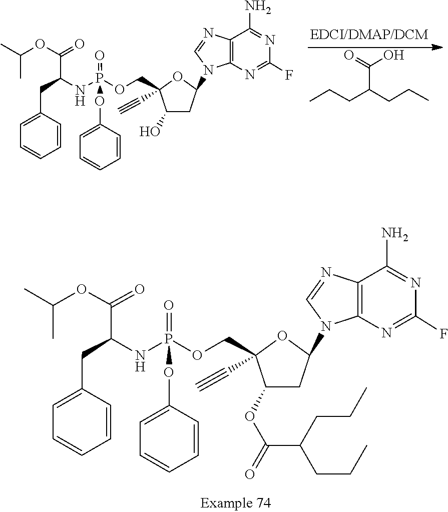

### (2R,3S,5R)-5-(6-Amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl 2-propylpentanoate

To a solution of 2-propylpentanoic acid (40.6 mg, 0.282 mmol) in DCM (6 mL) was added N,N-dimethylpyridin-4-amine (28.7 mg, 0.235 mmol) and 3-(((ethylimino)methylene)amino)-N,N-dimethylpropan-1-amine hydrochloride (90 mg, 0.470 mmol) and the resulting mixture was stirred for 0.5 h at ice-water bath temperature. Then isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.235 mmol) was added and the resulting mixture was stirred at 20° C. for 16 h. The LCMS showed the reaction was completed. The reaction mixture was concentrated and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-ethynyl-2-((((S)-(((S)-1-isopropoxy-1-oxo-3-phenylpropan-2-yl)amino)(phenoxy)phosphoryl)oxy)methyl)tetrahydrofuran-3-yl 2-propylpentanoate (114 mg, 0.149 mmol, 63.3% yield) as a white solid. LCMS (M+H)=765.2; Retention time (0.05% TFA)=1.94 min. ¹H NMR (400 MHz, CDCl₃) δ 7.99 (s, 1H), 7.24-7.19 (m, 10H), 6.39 (t, J=6.8 Hz, 1H), 5.94 (brs, 2H), 5.56 (dd, J=7.2, 4.8 Hz, 1H), 4.93 (quint, J=6.4 Hz, 1H), 4.32-4.24 (m, 2H), 4.13 (dd, J=11.2, 5.6 Hz, 1H), 3.64 (t, J=10.8 Hz, 1H), 2.99 (d, J=6.4 Hz, 2H), 2.73 (quint, J=6.8 Hz, 1H), 2.62 (s, 1H), 2.59 (ddd, J=13.2, 6.4, 4.8 Hz, 1H), 2.51-2.42 (m, 1H), 1.70-1.60 (m, 2H), 1.54-1.44 (m, 2H), 1.39-1.30 (m, 4H), 1.14 (dd, J=9.6, 6.4 Hz, 6H), 0.92 (dt, J=7.2, 2.0 Hz, 6H).

### Example 75

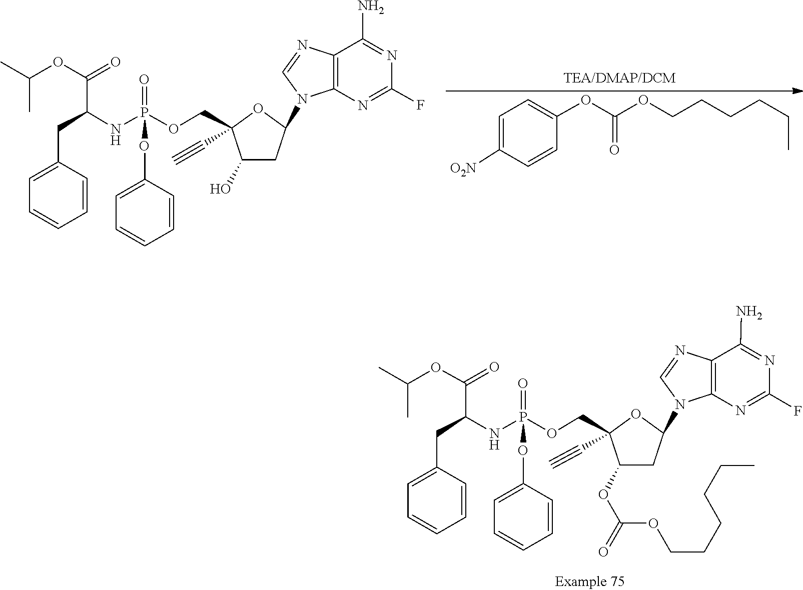 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1O ; CCCCCCOC(=O)Oc1ccc([N+](=O)[O-])cc1 ; C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCC]

### Isopropyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.235 mmol) in DCM (10 mL) was treated with triethylamine (0.098 mL, 0.705 mmol) and added DMAP (28.7 mg, 0.235 mmol) followed by hexyl (4-nitrophenyl) carbonate (251 mg, 0.940 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford isopropyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate (100 mg, 0.127 mmol, 54.0% yield) as white solid. LCMS (M+H)=766.7; Retention time (0.1% NH₄HCO₃)=2.007 min. ¹H NMR (400 MHz, CDCl₃) δ 7.95 (s, 1H), 7.26-7.07 (m, 10H), 6.38 (t, J=6.7 Hz, 1H), 5.93 (s, 2H), 5.48 (dd, J=6.9, 4.3 Hz, 1H), 4.93 (dt, J=12.5, 6.3 Hz, 1H), 4.28 (ddd, J=15.8, 9.8, 5.5 Hz, 2H), 4.24-4.09 (m, 3H), 3.66 (t, J=10.7 Hz, 1H), 2.99 (d, J=6.3 Hz, 2H), 2.85-2.59 (m, 3H), 1.76-1.70 (m, 2H), 1.45-1.22 (m, 6H), 1.14 (t, J=6.6 Hz, 6H), 0.89 (t, J=6.8 Hz, 3H).

### Example 76

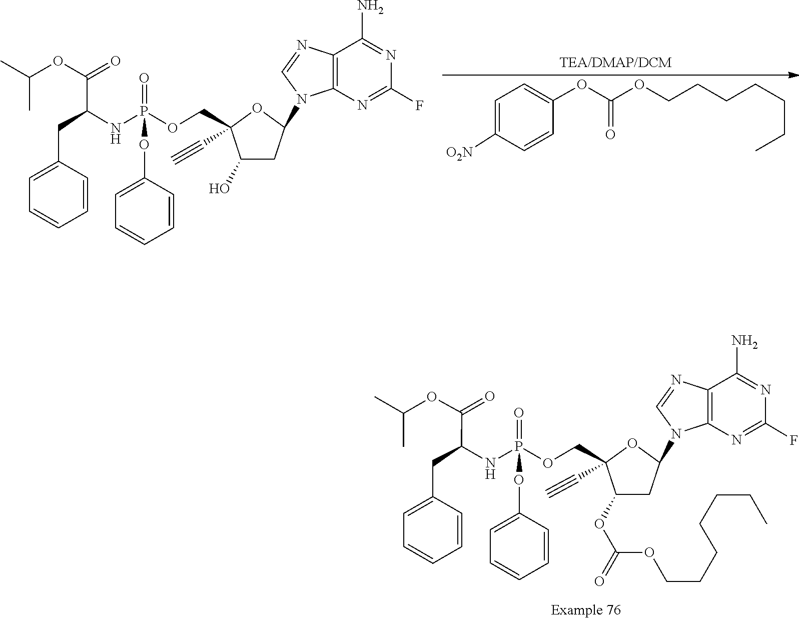 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1O ; CCCCCCCOC(=O)Oc1ccc([N+](=O)[O-])cc1 ; C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCC]

Isopropyl ((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)-L-phenylalaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.313 mmol) in DCM (10 mL) was treated with triethylamine (0.131 mL, 0.940 mmol), DMAP (38.3 mg, 0.313 mmol) followed by heptyl (4-nitrophenyl) carbonate (352 mg, 1.253 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford isopropyl ((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)-L-phenylalaninate (90 mg, 0.115 mmol, 36.8% yield) as white solid. LCMS (M+H)=780.7; Retention time (0.1% TFA)=1.947 min; purity (254 nm)=100%. ¹H NMR (400 MHz, CDCl₃) δ 7.96 (s, 1H), 7.26-7.07 (m, 10H), 6.38 (t, J=6.7 Hz, 1H), 5.92 (s, 2H), 5.48 (dd, J=6.9, 4.3 Hz, 1H), 5.03-4.82 (m, 1H), 4.37-4.25 (m, 2H), 4.26-4.10 (m, 3H), 3.64 (t, J=10.7 Hz, 1H), 2.99 (d, J=6.3 Hz, 2H), 2.85-2.59 (m, 3H), 1.72 (s, 2H), 1.40-1.24 (m, 8H), 1.14 (t, J=6.6 Hz, 6H), 0.89 (t, J=6.8 Hz, 3H).

### Example 77

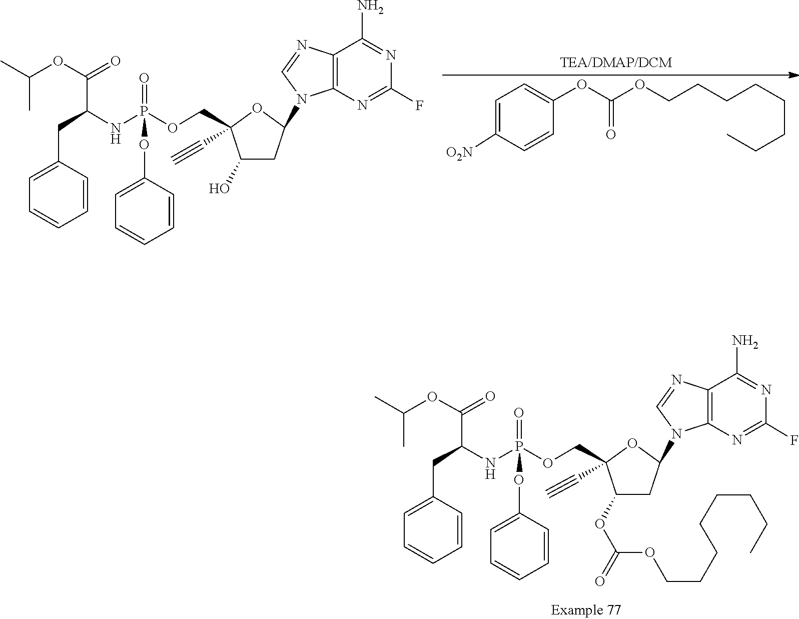 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1O ; CCCCCCCCOC(=O)Oc1ccc([N+](=O)[O-])cc1 ; C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCCC]

### Isopropyl ((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)-L-phenylalaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.313 mmol) in DCM (10 mL) was treated with triethylamine (0.131 mL, 0.940 mmol), DMAP (38.3 mg, 0.313 mmol) followed by 4-nitrophenyl octyl carbonate (370 mg, 1.253 mmol) and the mixture was stirred at RT for 2 days. TLC showed the reaction was completed. Added water (20 mL) then the mixture was extracted with DCM (20 L×3). The combined organic phase was washed with brine (80 mL), dried over Na₂SO₄ and concentrated in vacuum and the residue was concentrated and purified by silica gel chromatography (DCM/MeOH=100:0 to 20:1) to afford isopropyl ((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)-L-phenylalaninate (87 mg, 0.109 mmol, 34.9% yield) as white solid. LCMS (M+H)=794.7; Retention time (0.1% TFA)=2.000 min. ¹H NMR (400 MHz, CDCl₃) δ 7.95 (s, 1H), 7.26-7.07 (m, 10H), 6.38 (t, J=6.7 Hz, 1H), 5.93 (s, 2H), 5.48 (dd, J=6.9, 4.3 Hz, 1H), 4.93 (dt, J=12.5, 6.3 Hz, 1H), 4.28 (ddd, J=15.8, 9.8, 5.5 Hz, 2H), 4.24-4.09 (m, 3H), 3.66 (t, J=10.7 Hz, 1H), 2.99 (d, J=6.3 Hz, 2H), 2.85-2.59 (m, 3H), 1.76-1.70 (m, 2H), 1.45-1.22 (m, 10H), 1.14 (t, J=6.6 Hz, 6H), 0.89 (t, J=6.8 Hz, 3H).

### Example 78

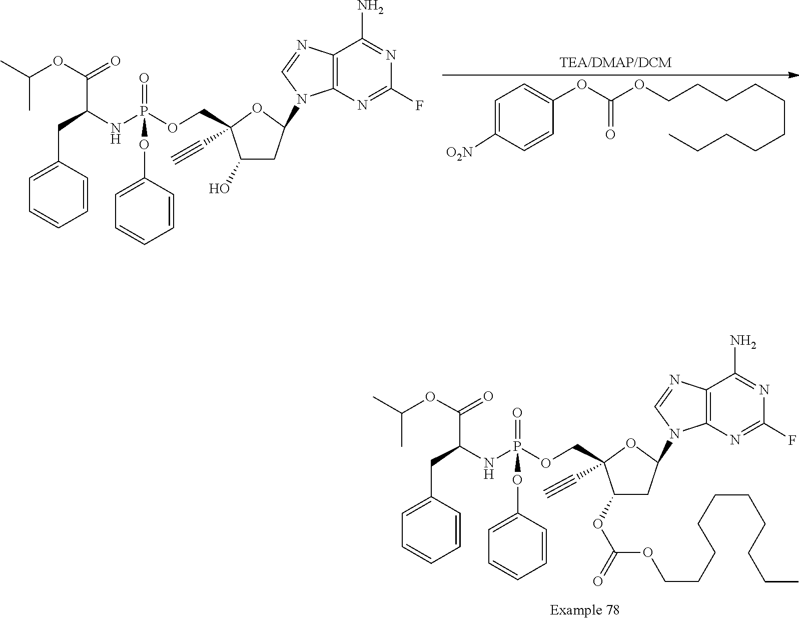 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1O ; CCCCCCCCCCOC(=O)Oc1ccc([N+](=O)[O-])cc1 ; C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCCCCC]

### Isopropyl ((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)-L-phenylalaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.235 mmol) in DCM (10 mL) was treated with triethylamine (0.098 mL, 0.705 mmol), DMAP (28.7 mg, 0.235 mmol) followed by decyl (4-nitrophenyl) carbonate (304 mg, 0.940 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford isopropyl ((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)-L-phenylalaninate (102.1 mg, 0.121 mmol, 51.3% yield) as white solid. LCMS (M+H)=822.7; Retention time (0.1% TFA)=2.118 min. ¹H NMR (400 MHz, CDCl₃) δ 7.95 (s, 1H), 7.26-7.06 (m, 10H), 6.38 (t, J=6.7 Hz, 1H), 5.85 (s, 2H), 5.48 (dd, J=6.9, 4.3 Hz, 1H), 5.04-4.89 (m, 1H), 4.29 (ddd, J=12.8, 8.0, 5.2 Hz, 2H), 4.17 (dt, J=11.1, 6.2 Hz, 3H), 3.61 (t, J=10.7 Hz, 1H), 2.99 (d, J=6.3 Hz, 2H), 2.81-2.59 (m, 3H), 1.69 (dd, J=14.6, 7.0 Hz, 2H), 1.44-1.22 (m, 14H), 1.14 (t, J=6.4 Hz, 6H), 0.88 (t, J=6.8 Hz, 3H).

### Example 79

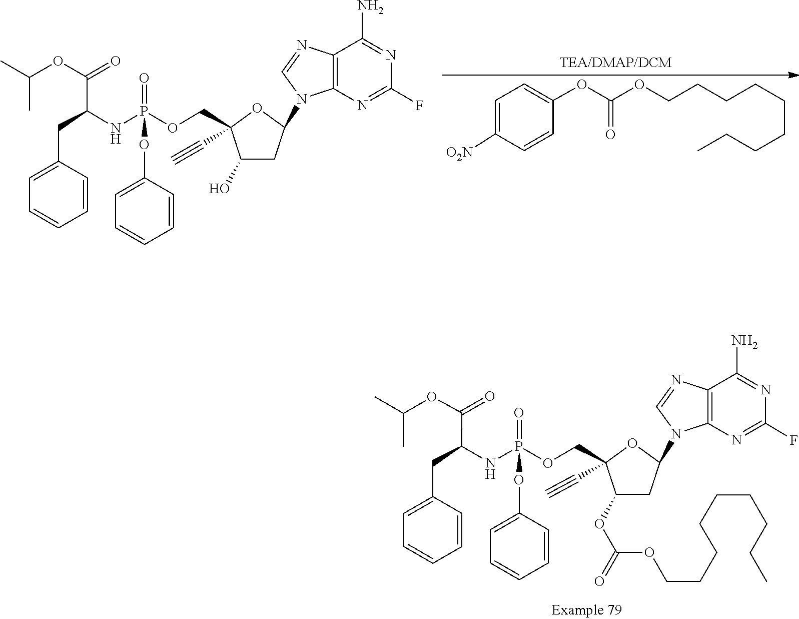 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1O ; CCCCCCCCCOC(=O)Oc1ccc([N+](=O)[O-])cc1 ; C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCCCC]

### Isopropyl ((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)-L-phenylalaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.235 mmol) in DCM (10 mL) was treated with triethylamine (0.098 mL, 0.705 mmol), DMAP (28.7 mg, 0.235 mmol) followed by 4-nitrophenyl nonyl carbonate (291 mg, 0.940 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford isopropyl ((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)-L-phenylalaninate (100 mg, 0.122 mmol, 51.8% yield) as white solid. LCMS (M+H)=808.7; Retention time (0.1% TFA)=2.507 min. ¹H NMR (400 MHz, CDCl₃) δ 8.10 (s, 1H), 7.26-7.06 (m, 10H), 6.38 (t, J=6.7 Hz, 1H), 6.35-6.02 (m, 2H), 5.58-5.35 (m, 1H), 5.04-4.83 (m, 1H), 4.32-4.25 (m, 2H), 4.18 (dt, J=17.4, 6.4 Hz, 3H), 3.71 (t, J=10.7 Hz, 1H), 3.10-2.87 (m, 2H), 2.83-2.59 (m, 3H), 1.76-1.67 (m, 2H), 1.31 (dd, J=24.9, 10.4 Hz, 12H), 1.15 (dd, J=7.3, 6.5 Hz, 6H), 0.88 (t, J=6.8 Hz, 3H).

### Example 80

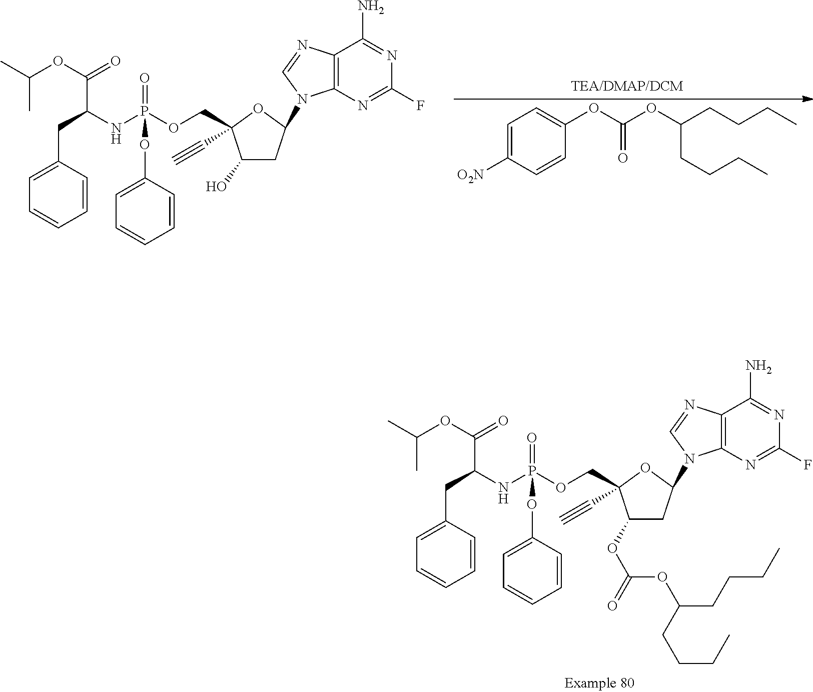 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1O ; CCCCC(CCCC)OC(=O)Oc1ccc([N+](=O)[O-])cc1 ; C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OC(CCCC)CCCC]

### Isopropyl ((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)-L-phenylalaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.313 mmol) and DMAP (38.3 mg, 0.313 mmol) in DCM (6 mL) was treated with triethylamine (0.131 mL, 0.940 mmol) followed by 4-nitrophenyl nonan-5-yl carbonate (388 mg, 1.253 mmol) and the mixture was stirred at 20° C. for 3 days. LCMS showed the reaction was finished. The reaction mixture was concentrated and purified by reverse phase chromatography (SepaFlash@ C18 column, BOSTON, 40 g, 0-100% CH3CN/10 mM aq. NH4HCO3) to give isopropyl ((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)-L-phenylalaninate (116 mg, 0.142 mmol, 45.4% yield) as a white solid. LCMS (M+H)=809.3; Retention time (0.05% TFA)=2.02 min. ¹H NMR (400 MHz, CDCl₃) δ 7.97 (s, 1H), 7.25-7.20 (m, 4H), 7.19-7.08 (m, 6H), 6.40 (t, J=6.8 Hz, 1H), 5.94 (brs, 2H), 5.45 (dd, J=6.8, 4.0 Hz, 1H), 4.94 (quint, J=6.4 Hz, 1H), 4.78-4.71 (m, 1H), 4.33-4.25 (m, 2H), 4.19 (dd, J=11.2, 5.6 Hz, 1H), 3.64 (t, J=10.8 Hz, 1H), 2.99 (d, J=6.4 Hz, 2H), 2.74 (quint, J=7.2 Hz, 1H), 2.67 (ddd, J=14.0, 6.0, 4.0 Hz, 1H), 2.63 (s, 1H), 1.67-1.55 (m, 4H), 1.40-1.30 (m, 8H), 1.14 (dd, J=8.8, 6.0 Hz, 6H), 0.91 (dd, J=14.0, 7.2 Hz, 6H).

### Example 81

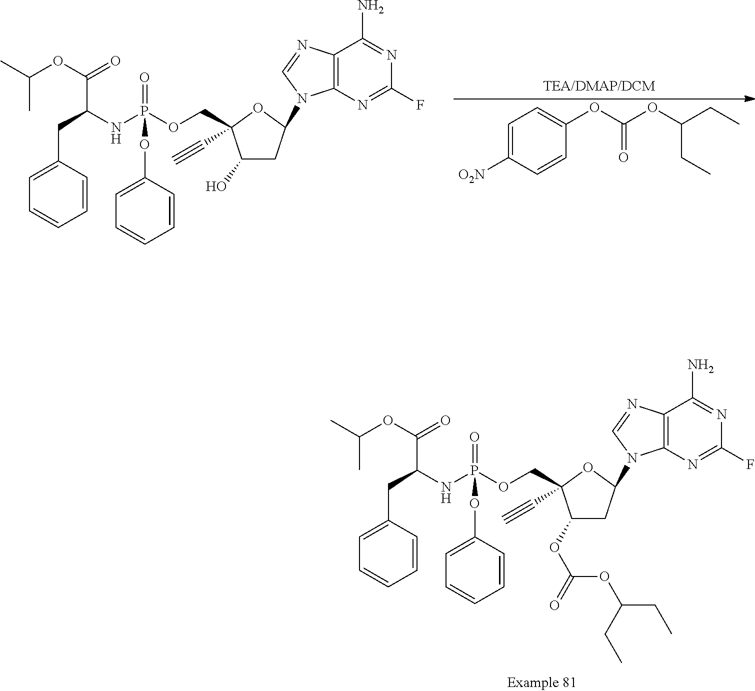 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1O ; CCC(CC)OC(=O)Oc1ccc([N+](=O)[O-])cc1 ; C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OC(CC)CC]

### Isopropyl ((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)-L-phenylalaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.313 mmol) and DMAP (38.3 mg, 0.313 mmol) in DCM (6 mL) was treated with triethylamine (0.131 mL, 0.940 mmol) and followed by 4-nitrophenyl pentan-3-yl carbonate (317 mg, 1.253 mmol) and the mixture was stirred at 20° C. for 2 days. LCMS showed the reaction was finished. The reaction mixture was concentrated and the residue was purified by reverse phase chromatography (SepaFlash@ C18 column, BOSTON, 40 g, 0-100% CH3CN/10 mM aq. NH4HCO3) to give isopropyl ((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)-L-phenylalaninate (130 mg, 0.169 mmol, 54.1% yield) as a white solid. LCMS (M+H)=753.2; Retention time (0.05% TFA)=1.84 min. ¹H NMR (400 MHz, CDCl₃) δ 7.95 (s, 1H), 7.25-7.08 (m, 10H), 6.40 (t, J=6.8 Hz, 1H), 5.82 (brs, 2H), 5.47 (dd, J=7.2, 4.0 Hz, 1H), 4.94 (quint, J=6.4 Hz, 1H), 4.64 (quint, J=6.4 Hz, 1H), 4.33-4.25 (m, 2H), 4.19 (dd, J=11.2, 5.6 Hz, 1H), 3.60 (t, J=10.8 Hz, 1H), 2.99 (d, J=6.4 Hz, 2H), 2.76 (quint, J=7.2 Hz, 1H), 2.71-2.66 (m, 1H), 2.65 (s, 1H), 1.71-1.64 (m, 4H), 1.14 (dd, J=8.0, 6.4 Hz, 6H), 0.95 (dt, J=7.6, 2.0 Hz, 6H).

### Example 82

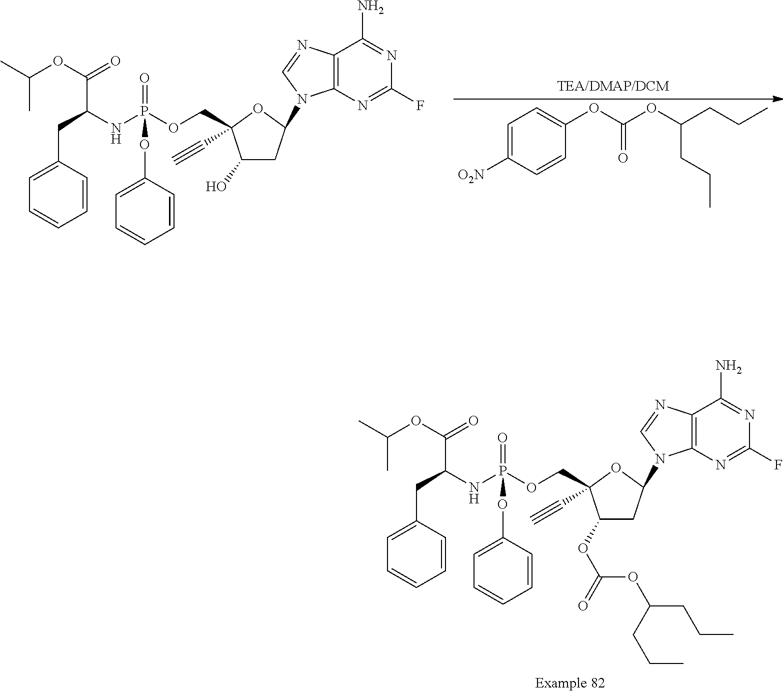 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1O ; CCCC(CCC)OC(=O)Oc1ccc([N+](=O)[O-])cc1 ; C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OC(CCC)CCC]

### Isopropyl ((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)-L-phenylalaninate

A mixture of isopropyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.313 mmol) and DMAP (38.3 mg, 0.313 mmol) in DCM (6 mL) was treated with triethylamine (0.131 mL, 0.940 mmol) and followed by heptan-4-yl (4-nitrophenyl) carbonate (352 mg, 1.253 mmol) and the mixture was stirred at 20° C. for 2 days. LCMS showed the reaction was finished. The reaction mixture was concentrated and the residue was purified by reverse phase chromatography (SepaFlash@ 018 column, BOSTON, 40 g, 0-100% CH3CN/10 mM aq. NH4HCO3) to give isopropyl ((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)-L-phenylalaninate (123 mg, 0.155 mmol, 49.5% yield) as a white solid. LCMS (M+H)=781.2; Retention time (0.05% TFA)=1.93 min ¹H NMR (400 MHz, CDCl₃) δ 7.97 (s, 1H), 7.26-7.08 (in, 1 OH), 6.39 (t, J=6.8 Hz, 1H), 5.94 (brs, 2H), 5.45 (dd, J=7.2, 4.0 Hz, 1H), 4.94 (quint, J=6.4 Hz, 1H), 4.82-4.74 (m, 1H), 4.33-4.24 (m, 2H), 4.19 (dd, J=10.8, 5.6 Hz, 1H), 3.64 (t, J=10.8 Hz, 1H), 2.99 (d, J=6.4 Hz, 2H), 2.75 (quint, J=7.2 Hz, 1H), 2.66 (ddd, J=14.0, 6.0, 4.0 Hz, 1H), 2.64 (s, 1H), 1.68-1.52 (m, 4H), 1.48-1.30 (m, 4H), 1.14 (dd, J=8.8, 6.4 Hz, 6H), 0.93 (dd, J=14.0, 7.2 Hz, 6H).

### Example 83

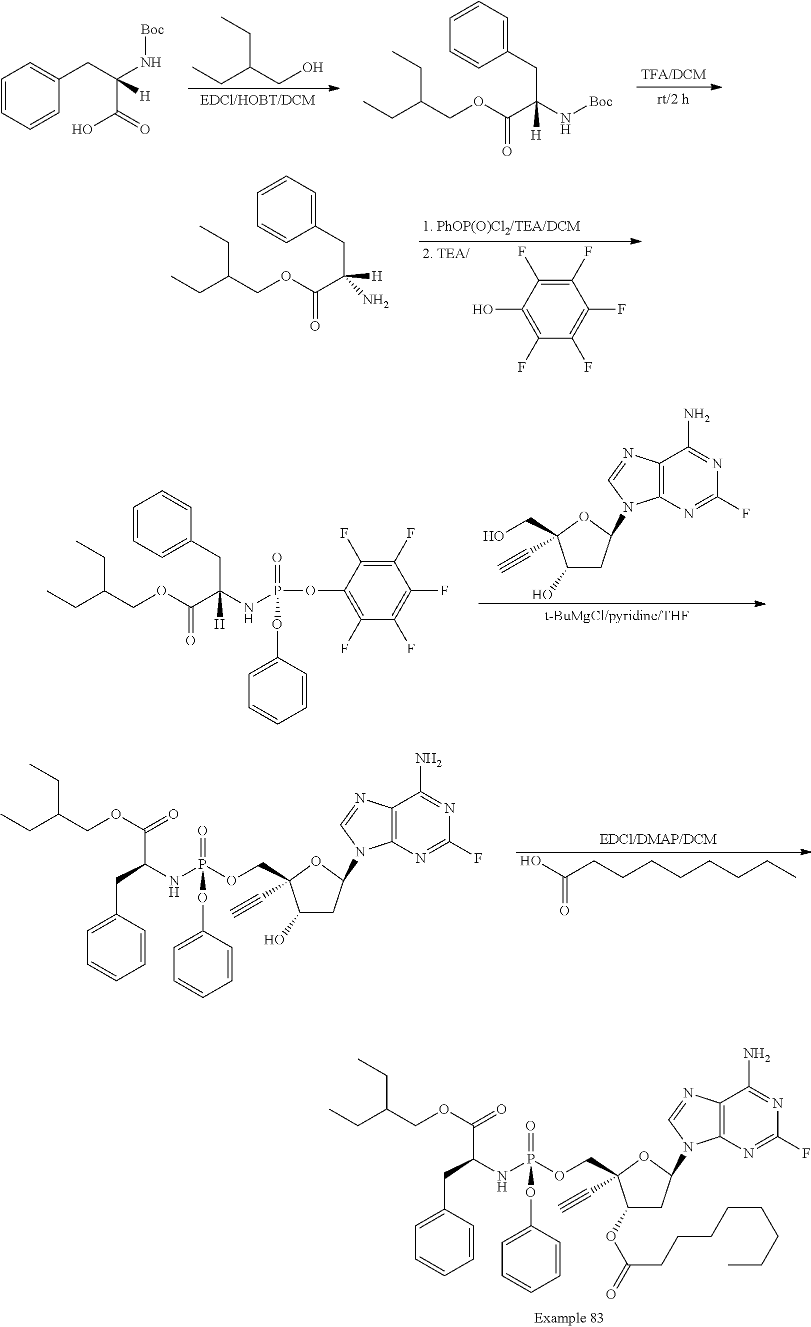

### Step 1: 2-Ethylbutyl (tert-butoxycarbonyl)-L-phenylalaninate

A mixture of 2-ethylbutan-1-ol (5.08 g, 49.8 mmol)), HOBt (3.81 g, 24.88 mmol), EDC (14.31 g, 74.6 mmol) in DCM (100 mL) was added DIEA (26.1 mL, 149 mmol) at −10° C. After stirring for 30 min, (tert-butoxycarbonyl)-L-phenylalanine (13.2 g, 49.8 mmol) in DCM (10 mL) was added and stirred at 25° C. for 2 h. TLC and LCMS showed the presence of new product. The reaction was diluted with water (50 mL) and extracted with EtOAc (50 mL×3). The combined organic layers were washed with brine (50 mL), dried with Na₂SO₄, filtered and concentrated in vacuum to give the residue. It was purified by flash column chromatography (pet. ether/EtOAc=100/0 to 20/1) to give 2-ethylbutyl (tert-butoxycarbonyl)-L-phenylalaninate (12.5 g, 35.8 mmol, 71.9% yield) as colorless oil. LCMS (M-Boc+H)=250.0; Retention time (0.05% TFA)=2.589 min.

### Step 2: 2-Ethylbutyl L-phenylalaninate

To a solution of 2-ethylbutyl (tert-butoxycarbonyl)-L-phenylalaninate (12.5 g, 35.8 mmol) in DCM (200 mL) was added TFA (27.6 mL, 358 mmol) under N₂. The reaction was stirred for 2 h at 25° C. under N₂. TLC showed the reaction was completed. The reaction was concentrated in vacuum and the residue was diluted with water (50 mL). The pH was adjusted to 7 with a solution of NaHCO₃and extracted with EtOAc (50 mL×3). The combined organic layers were washed with brine (50 mL), dried over sodium sulfate and evaporated. The residue was purified by flash column chromatography (DCM/MeOH=100/0 to 20/1) to give 2-ethylbutyl L-phenylalaninate (8.5 g, 34.1 mmol, 95% yield) as pale yellow oil. ¹H NMR (400 MHz, CDCl₃) δ 7.25 (ddd, J=25.3, 16.4, 7.2 Hz, 5H), 4.38 (s, 2H), 4.07-3.93 (m, 2H), 3.87 (t, J=6.2 Hz, 1H), 3.12 (dd, J=13.7, 5.6 Hz, 1H), 2.99 (dd, J=13.7, 7.3 Hz, 1H), 1.46 (dq, J=12.2, 6.1 Hz, 1H), 1.28 (dd, J=14.2, 7.2 Hz, 4H), 0.86 (t, J=7.4 Hz, 6H).

### Step 3: 2-Ethylbutyl ((S)-(perfluorophenoxy)(phenoxy)phosphoryl)-L-phenylalaninate

To a solution of 2-ethylbutyl L-phenylalaninate (8.5 g, 34.1 mmol) in anhydrous DCM (35 mL) was added dropwise triethylamine (9.95 mL, 71.6 mmol) at −70° C. over 15 min. To this mixture was added a solution of phenyl phosphorodichloridate (7.12 g, 33.7 mmol) in anhydrous DCM (35 mL) over 1 h. The reaction mixture was stirred at this temperature for additional 30 min and then allowed to warm to 0° C. over 2 h and stirred for additional 1 h. To this mixture was added a solution of 2,3,4,5,6-pentafluorophenol (6.21 g, 33.7 mmol) and triethylamine (5.21 mL, 37.5 mmol) in DCM (30 mL) over 20 min and stirred at 0° C. for 4 h. LCMS showed the reaction was completed. The white solid (triethylaminehydrochloride) was filtered off and washed with DCM (25 mL). The filtrate was concentrated under reduced pressure, the residue was triturated with TBME (150 mL), and the triethylaminehydrochloride salt was removed by filtration. The cake was washed with TBME (2×25 mL), and the combined filtrate was concentrated under reduced pressure. The residue was triturated with 20% EtOAc in hexanes (100 mL) and solids collected by filtration to give 2-ethylbutyl ((S)-(perfluorophenoxy)(phenoxy)phosphoryl)-L-phenylalaninate (4.2 g, 7.35 mmol, 21.56% yield) as a white solid (>98% de as determined by P³¹NMR). LCMS (M+H)=571; Retention time (0.05% TFA)=2.384 min. ³¹P NMR (162 MHz, DMSO-d₆) δ−1.54.

### Step 4: 2-Ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a solution of (2R,3S,5R)-5-(6-amino-2-fluoro-9H-purin-9-yl)-2-ethynyl-2-(hydroxymethyl)tetrahydrofuran-3-ol (0.3 g, 1.023 mmol) in THF (60 mL) and pyridine (15.0 mL) was added dropwise tert-butylmagnesium chloride (2.148 mL, 2.148 mmol) at 0° C. Then, the reaction was stirred at 25° C. for 30 min. A solution of 2-ethylbutyl ((S)-(perfluorophenoxy)(phenoxy)phosphoryl)-L-phenylalaninate (0.877 g, 1.534 mmol) in THF (20 mL) was added dropwise to the above solution at −15° C. and stirred at −15° C. for 4 h. TLC showed the reaction was completed. The reaction mixture was diluted with 2 M HCl (30 mL) and extracted with EtOAc (50 mL×3). The combined organic layers were washed with a solution of NaHCO₃ (50 mL), brine (20 mL), dried with Na₂SO₄, filtered and concentrated in vacuum to give the crude product which was purified by prep-HPLC (basic) to give 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.294 mmol, 28.7% yield) as white solid. LCMS (M+H)=681; Retention time (0.05% TFA)=1.760 min.

### Step 5: (2R,3S,5R)-5-(6-Amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl nonanoate

To a solution of nonanoic acid (45.3 mg, 0.286 mmol) in DCM (10 mL) was added N,N-dimethylpyridin-4-amine (26.9 mg, 0.220 mmol) and 3-(((ethylimino)methylene)amino)-N,N-dimethylpropan-1-amine hydrochloride (84 mg, 0.441 mmol) and the resulting mixture was stirred for 0.5 h at ice-water bath temperature. Then, 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.220 mmol) was added and the resulting mixture was stirred at 25° C. for 16 h. The LCMS showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl nonanoate (131 mg, 0.156 mmol, 71.0% yield) as a white solid. LCMS: LCMS (M+H)=821.3; Retention time (0.05% TFA)=2.157 min. ¹H NMR (400 MHz, CDCl₃) δ 8.02 (s, 1H), 7.26-7.06 (m, 10H), 6.39 (d, J=6.9 Hz, 1H), 6.37-5.90 (m, 2H), 5.57 (s, 1H), 4.34 (t, J=9.9 Hz, 1H), 4.23 (d, J=6.3 Hz, 1H), 4.14 (d, J=5.7 Hz, 1H), 3.96 (dd, J=5.8, 1.7 Hz, 2H), 3.77 (s, 1H), 3.09-2.92 (m, 2H), 2.70-2.50 (m, 3H), 2.40 (t, J=7.6 Hz, 2H), 1.79-1.59 (m, 2H), 1.49-1.14 (m, 15H), 0.86 (dt, J=14.9, 7.0 Hz, 9H).

### Example 84

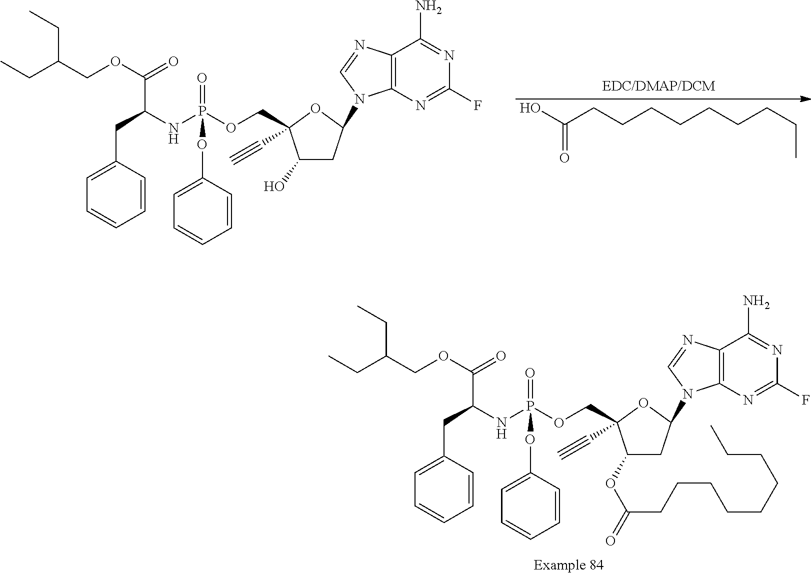 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCCCCCCCC(=O)O ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCC]

### (2R,3S,5R)-5-(6-Amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl decanoate

To a solution of decanoic acid (49.4 mg, 0.286 mmol) in DCM (10 mL) was added N,N-dimethylpyridin-4-amine (26.9 mg, 0.220 mmol) and 3-(((ethylimino)methylene)amino)-N,N-dimethylpropan-1-amine hydrochloride (84 mg, 0.441 mmol) and the resulting mixture was stirred for 0.5 h at ice-water bath temperature. Then, 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.220 mmol) was added and the resulting mixture was stirred at 25° C. for 16 h. The LCMS showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to give (2R,3S,5R)-5-(6-amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl decanoate (133 mg, 0.159 mmol, 72.3% yield) as a white solid. LCMS (M+H)=835.3; Retention time (0.05% TFA)=2.235 min. ¹H NMR (400 MHz, CDCl₃) δ 7.99 (s, 1H), 7.26-7.07 (m, 10H), 6.40 (s, 1H), 6.14 (s, 2H), 5.57 (d, J=2.4 Hz, 1H), 4.40-4.29 (m, 1H), 4.23 (d, J=6.3 Hz, 1H), 4.14 (d, J=5.7 Hz, 1H), 3.96 (dd, J=5.8, 2.0 Hz, 2H), 3.72 (s, 1H), 3.08-2.90 (m, 2H), 2.72-2.52 (m, 3H), 2.40 (t, J=7.6 Hz, 2H), 1.66 (d, J=7.4 Hz, 2H), 1.41 (dd, J=12.5, 6.2 Hz, 1H), 1.37-1.14 (m, 16H), 0.88 (t, J=6.8 Hz, 3H), 0.83 (t, J=7.4 Hz, 6H).

### Example 85

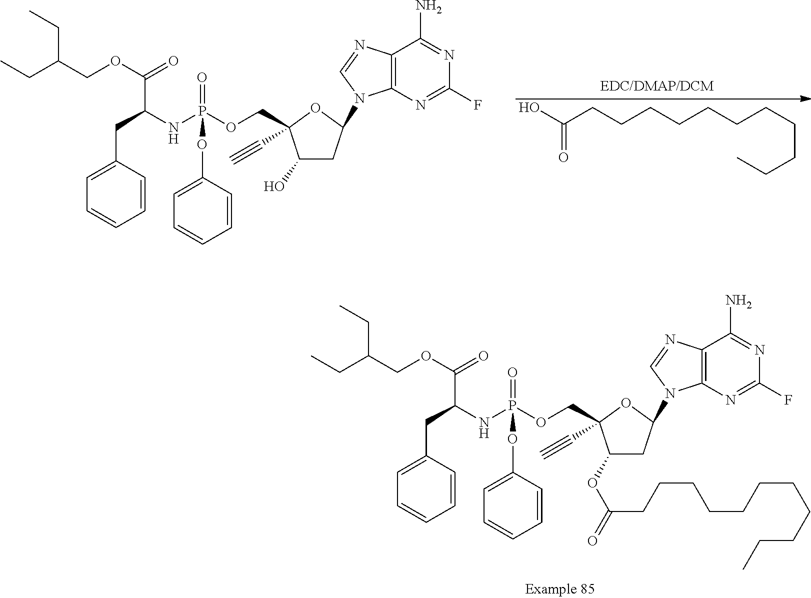 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCCCCCCCCCC(=O)O ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCC]

### (2R,3S,5R)-5-(6-Amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl dodecanoate

To a solution of dodecanoic acid (57.4 mg, 0.286 mmol) in DCM (10 mL) was added N,N-dimethylpyridin-4-amine (26.9 mg, 0.220 mmol) and 3-(((ethylimino)methylene)amino)-N,N-dimethylpropan-1-amine hydrochloride (84 mg, 0.441 mmol) and the resulting mixture was stirred for 0.5 h at ice-water bath temperature. Then, 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.220 mmol) was added and the resulting mixture was stirred at 25° C. for 4 h. The LCMS showed the reaction was completed. The reaction was concentrated in vacuum and the residue was purified by reverse phase chromatography (SepaFlash@ C18 column, BOSTON, 40 g, 0-100% CH3CN/10 mM aq. NH4HCO3) to obtain (2R,3S,5R)-5-(6-amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl dodecanoate (147 mg, 0.165 mmol, 75.0% yield) as a white solid. LCMS (M+H)=863.4; Retention time (0.05% TFA)=2.419 min. ¹H NMR (400 MHz, CDCl₃) δ 8.02 (s, 1H), 7.21 (dd, J=41.8, 19.0 Hz, 10H), 6.92-6.50 (m, 2H), 6.41 (s, 1H), 5.60 (s, 1H), 4.36 (s, 1H), 4.27 (s, 1H), 4.17 (s, 1H), 4.06 (s, 1H), 3.97 (s, 2H), 3.02 (s, 2H), 2.64 (s, 3H), 2.41 (s, 2H), 1.68 (s, 2H), 1.28 (s, 21H), 0.97-0.73 (m, 9H).

### Example 86

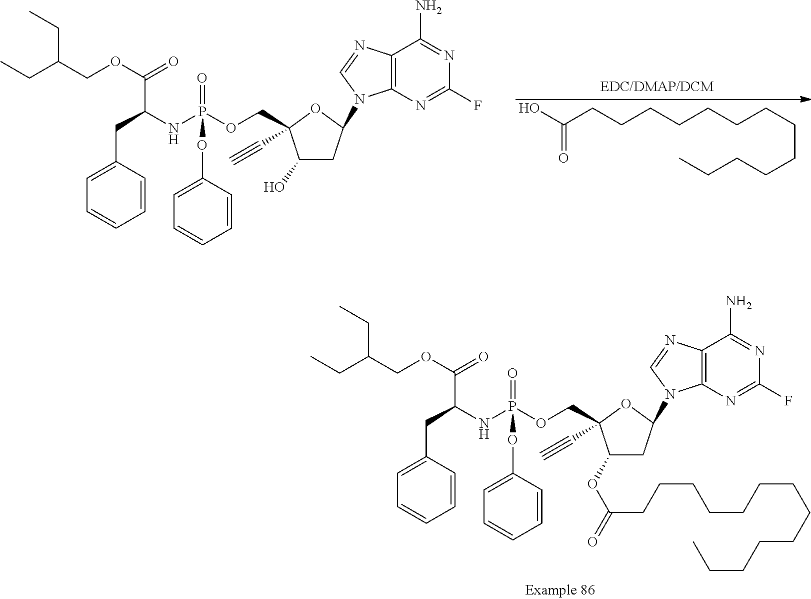 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCCCCCCCCCCCC(=O)O ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCCCC]

### (2R,3S,5R)-5-(6-Amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl tetradecanoate

To a solution of tetradecanoic acid (65.4 mg, 0.286 mmol) in DCM (10 mL) was added N,N-dimethylpyridin-4-amine (26.9 mg, 0.220 mmol) and 3-(((ethylimino)methylene)amino)-N,N-dimethylpropan-1-amine hydrochloride (84 mg, 0.441 mmol) and the resulting mixture was stirred for 0.5 h at ice-water bath temperature. Then, 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.220 mmol) was added and the resulting mixture was stirred at 25° C. for 4 h. The LCMS showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to obtain (2R,3S,5R)-5-(6-amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl tetradecanoate (117 mg, 0.127 mmol, 57.8% yield) as a white solid. LCMS (M+H)=891.5; Retention time (0.05% TFA)=2.678 min. ¹H NMR (400 MHz, CDCl₃) δ 8.02 (s, 1H), 7.18 (dt, J=22.0, 12.8 Hz, 10H), 6.91 (s, 2H), 6.39 (s, 1H), 5.59 (s, 1H), 4.25 (t, J=34.3 Hz, 4H), 3.96 (s, 2H), 3.01 (s, 2H), 2.60 (d, J=18.0 Hz, 3H), 2.40 (s, 2H), 1.67 (s, 2H), 1.26 (s, 25H), 0.85 (d, J=26.3 Hz, 9H).

### Example 87

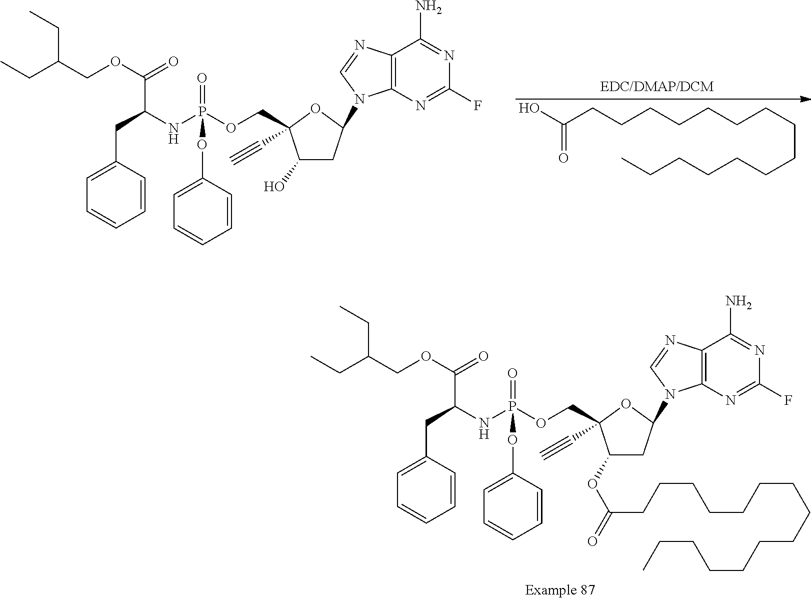 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCCCCCCCCCCCCCC(=O)O ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCCCCCC]

### (2R,3S,5R)-5-(6-Amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl palmitate

To a solution of palmitic acid (73.5 mg, 0.286 mmol) in DCM (10 mL) was added N,N-dimethylpyridin-4-amine (26.9 mg, 0.220 mmol) and 3-(((ethylimino)methylene)amino)-N,N-dimethylpropan-1-amine hydrochloride (84 mg, 0.441 mmol) and the resulting mixture was stirred for 0.5 h at ice-water bath temperature. Then, 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.220 mmol) was added and the resulting mixture was stirred at 25° C. for 4 h. LCMS showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to obtain (2R,3S,5R)-5-(6-amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl palmitate (124 mg, 0.132 mmol, 60.0% yield) as a white solid. LCMS: Retention time (0.05% TFA)=3.309 min. ¹H NMR (400 MHz, CDCl₃) δ 8.01 (s, 1H), 7.26-7.05 (m, 10H), 6.67 (s, 2H), 6.39 (t, J=6.7 Hz, 1H), 5.67-5.44 (m, 1H), 4.34 (s, 1H), 4.25 (d, J=6.3 Hz, 1H), 4.16 (d, J=5.6 Hz, 1H), 4.06 (s, 1H), 3.96 (d, J=5.6 Hz, 2H), 3.01 (t, J=5.7 Hz, 2H), 2.76-2.49 (m, 3H), 2.39 (dd, J=16.0, 8.4 Hz, 3H), 1.79-1.53 (m, 2H), 1.45-1.16 (m, 29H), 0.85 (dt, J=14.8, 6.9 Hz, 9H).

### Example 88

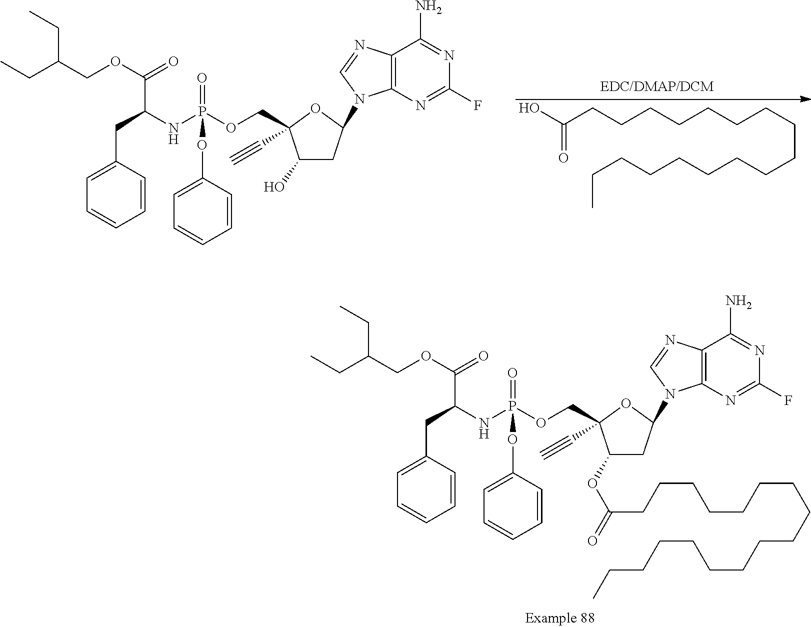 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCCCCCCCCCCCCCCCC(=O)O ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCCCCCCCC]

### (2R,3S,5R)-5-(6-Amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl stearate

To a solution of stearic acid (217 mg, 0.764 mmol) in DCM (10 mL) was added N,N-dimethylpyridin-4-amine (71.8 mg, 0.588 mmol) and 3-(((ethylimino)methylene)amino)-N,N-dimethylpropan-1-amine hydrochloride (225 mg, 1.175 mmol) and the resulting mixture was stirred for 0.5 h at ice-water bath temperature. Then, 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (400 mg, 0.588 mmol) was added and the resulting mixture was stirred at 25° C. for 16 h. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to obtain (2R,3S,5R)-5-(6-amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl stearate (200 mg, 0.205 mmol, 34.9% yield) as a light yellow semi-solid. LCMS: Retention time (0.05% TFA)=4.355 min. ¹H NMR (400 MHz, CDCl₃) δ 8.00 (s, 1H), 7.25-7.07 (m, 10H), 6.40 (t, J=6.7 Hz, 1H), 6.01 (s, 2H), 5.65-5.53 (m, 1H), 4.34 (s, 1H), 4.29-4.20 (m, 1H), 4.13 (dd, J=11.0, 5.6 Hz, 1H), 3.96 (dd, J=5.8, 2.4 Hz, 2H), 3.65 (s, 1H), 3.00 (d, J=6.2 Hz, 2H), 2.73-2.47 (m, 3H), 2.40 (t, J=7.6 Hz, 2H), 1.66 (d, J=7.4 Hz, 2H), 1.48-1.16 (m, 33H), 0.85 (dt, J=14.9, 7.0 Hz, 9H).

### Example 89

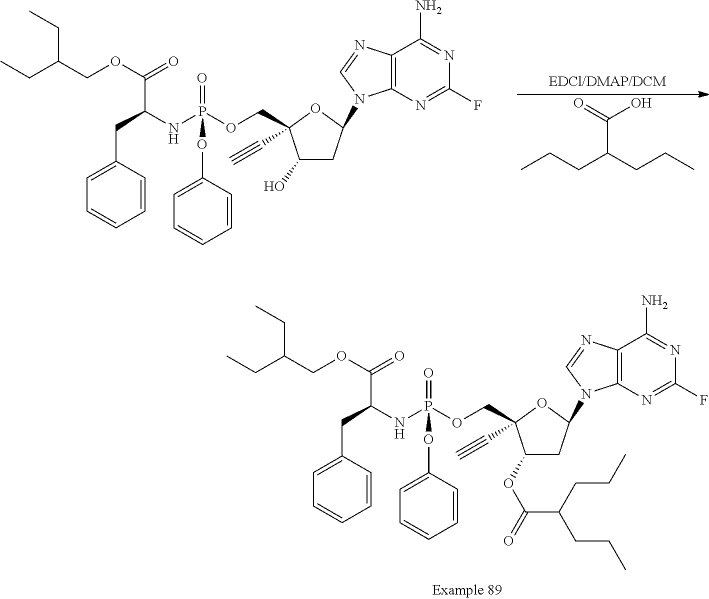

### (2R,3S,5R)-5-(6-Amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl 2-propylpentanoate

To a solution of 2-propylpentanoic acid (49.6 mg, 0.344 mmol) in DCM (10 mL) was added N,N-dimethylpyridin-4-amine (32.3 mg, 0.264 mmol) and 3-(((ethylimino)methylene)amino)-N,N-dimethylpropan-1-amine hydrochloride (101 mg, 0.529 mmol) and the resulting mixture was stirred for 0.5 h at ice-water bath temperature. Then, 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (180 mg, 0.264 mmol) was added and the resulting mixture was stirred at 25° C. for 4 h. The LCMS showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to obtain (2R,3S,5R)-5-(6-amino-2-fluoro-9H-purin-9-yl)-2-((((S)-(((S)-1-(2-ethylbutoxy)-1-oxo-3-phenylpropan-2-yl)amino)(phenoxy)phosphoryl)oxy)methyl)-2-ethynyltetrahydrofuran-3-yl 2-propylpentanoate (140 mg, 0.168 mmol, 63.4% yield) as a white solid. LCMS (M+H)=807.3; Retention time (0.05% TFA)=2.096 min. ¹H NMR (400 MHz, CDCl₃) δ 8.13 (s, 1H), 7.21 (dd, J=13.9, 7.1 Hz, 5H), 7.17-7.07 (m, 5H), 6.39 (s, 1H), 6.32-5.74 (m, 2H), 5.54 (d, J=7.0 Hz, 1H), 4.34 (s, 1H), 4.24 (d, J=6.4 Hz, 1H), 4.14 (d, J=5.9 Hz, 1H), 3.96 (dd, J=5.8, 2.7 Hz, 2H), 3.69 (s, 1H), 3.10-2.91 (m, 2H), 2.65 (d, J=16.9 Hz, 3H), 2.47 (s, 1H), 1.66 (s, 2H), 1.55-1.15 (m, 11H), 0.92 (td, J=7.2, 1.8 Hz, 6H), 0.83 (t, J=7.4 Hz, 6H).

### Example 90

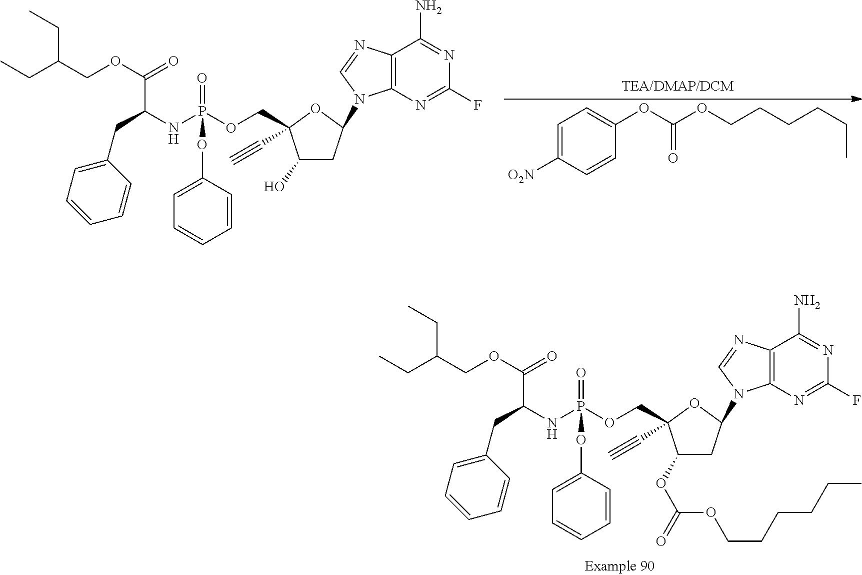 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCCCCOC(=O)Oc1ccc([N+](=O)[O-])cc1 ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCC]

### 2-Ethylbutyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (180 mg, 0.264 mmol) in DCM (10 mL) was treated with triethylamine (0.111 mL, 0.793 mmol), DMAP (32.3 mg, 0.264 mmol) followed by hexyl (4-nitrophenyl) carbonate (283 mg, 1.058 mmol) and the mixture was stirred at RT for 2 days. LCMS showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford 2-ethylbutyl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate (101 mg, 0.121 mmol, 45.8% yield) as white solid. LCMS (M+H)=809; Retention time (0.1% TFA)=2.022 min. ¹H NMR (400 MHz, CDCl₃) δ 7.94 (s, 1H), 7.26-7.19 (m, 5H), 7.13 (dd, J=15.5, 7.5 Hz, 5H), 6.38 (s, 1H), 5.99-5.64 (m, 2H), 5.48 (s, 1H), 4.27 (d, J=6.2 Hz, 2H), 4.19 (dd, J=13.8, 8.0 Hz, 3H), 3.95 (dd, J=5.8, 2.7 Hz, 2H), 3.58 (s, 1H), 3.01 (s, 2H), 2.71 (d, J=35.7 Hz, 3H), 1.70 (d, J=7.7 Hz, 2H), 1.31 (dd, J=40.4, 19.2 Hz, 11H), 0.96-0.74 (m, 9H).

### Example 91

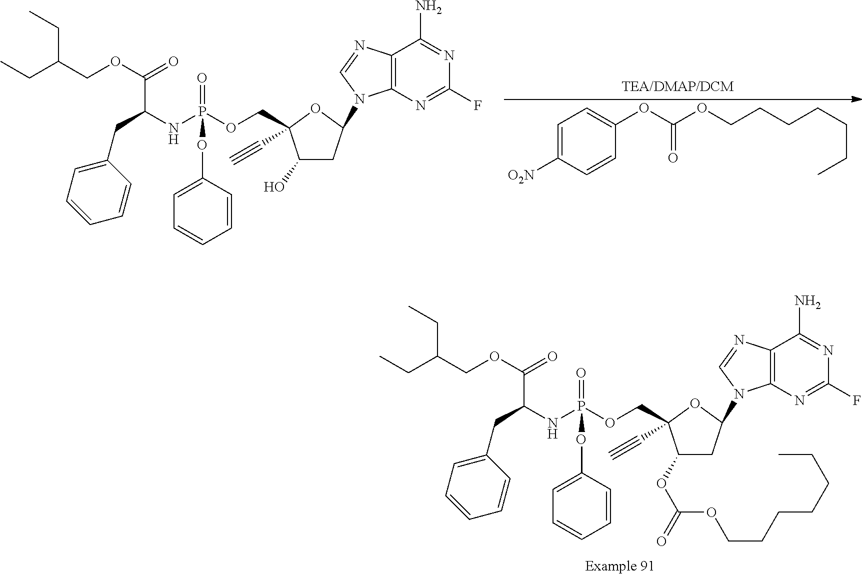 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCCCCCOC(=O)Oc1ccc([N+](=O)[O-])cc1 ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCC]

### 2-Ethylbutyl ((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)-L-phenylalaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (180 mg, 0.264 mmol) in DCM (10 mL) was treated with triethylamine (0.111 mL, 0.793 mmol), DMAP (32.3 mg, 0.264 mmol) followed by heptyl (4-nitrophenyl) carbonate (298 mg, 1.058 mmol) and the mixture was stirred at RT for 2 days. LCMS showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3). ¹H NMR: N78913-50-A1, Succeed to afford 2-ethylbutyl ((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)-L-phenylalaninate (95 mg, 0.113 mmol, 42.8% yield) as white solid. LCMS (M+H)=823.3; Retention time (0.05% TFA)=2.076 min. ¹H NMR (400 MHz, CDCl₃) δ 7.94 (s, 1H), 7.22 (dd, J=12.5, 7.0 Hz, 5H), 7.13 (dd, J=15.4, 7.5 Hz, 5H), 6.38 (t, J=6.7 Hz, 1H), 6.06-5.66 (m, 2H), 5.54-5.40 (m, 1H), 4.43-4.26 (m, 2H), 4.17 (dt, J=11.0, 6.2 Hz, 3H), 3.95 (dd, J=5.8, 2.6 Hz, 2H), 3.61 (s, 1H), 3.00 (d, J=6.4 Hz, 2H), 2.84-2.60 (m, 3H), 1.77-1.66 (m, 2H), 1.51-1.15 (m, 13H), 1.00-0.75 (m, 9H).

### Example 92

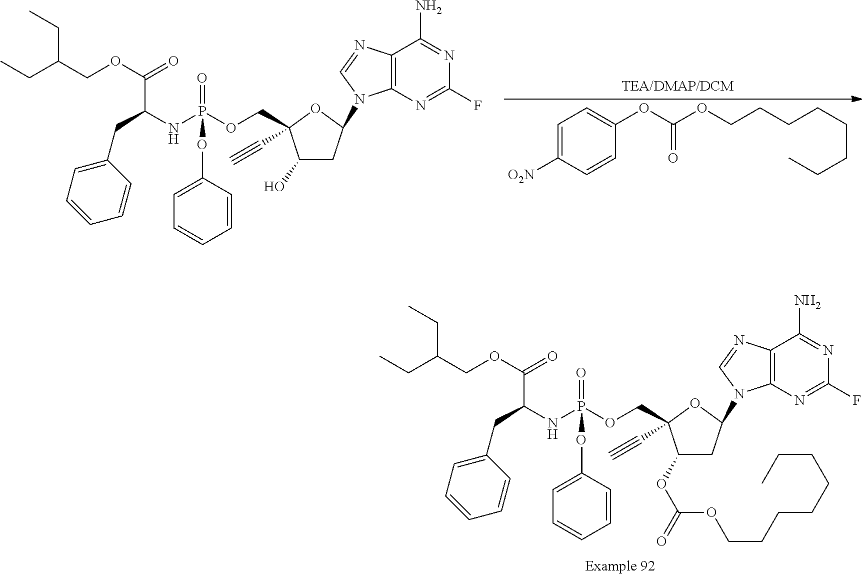 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCCCCCCOC(=O)Oc1ccc([N+](=O)[O-])cc1 ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCCC]

### 2-Ethylbutyl ((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)-L-phenylalaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (180 mg, 0.264 mmol) in DCM (10 mL) was treated with triethylamine (0.111 mL, 0.793 mmol), DMAP (32.3 mg, 0.264 mmol) followed by 4-nitrophenyl octyl carbonate (312 mg, 1.058 mmol) and the mixture was stirred at RT for 2 days. LCMS showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3). ¹H NMR: N78913-51-A1; Succeed to afford 2-ethylbutyl ((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)-L-phenylalaninate (127 mg, 0.150 mmol, 56.8% yield) as white solid. LCMS (M+H)=837.2; Retention time (0.05% TFA)=2.133 min; purity (254 nm)=99%. ¹H NMR (400 MHz, CDCl₃) δ 8.24-8.05 (m, 1H), 7.22 (dd, J=14.3, 7.2 Hz, 5H), 7.12 (dd, J=13.6, 7.0 Hz, 5H), 6.38 (s, 1H), 6.31-5.60 (m, 2H), 5.46 (s, 1H), 4.44-4.09 (m, 5H), 3.96 (dd, J=5.8, 2.6 Hz, 2H), 3.62 (s, 1H), 3.01 (d, J=6.5 Hz, 2H), 2.79-2.59 (m, 3H), 1.70 (d, J=7.8 Hz, 2H), 1.28 (s, 15H), 0.99-0.73 (m, 9H).

### Example 93

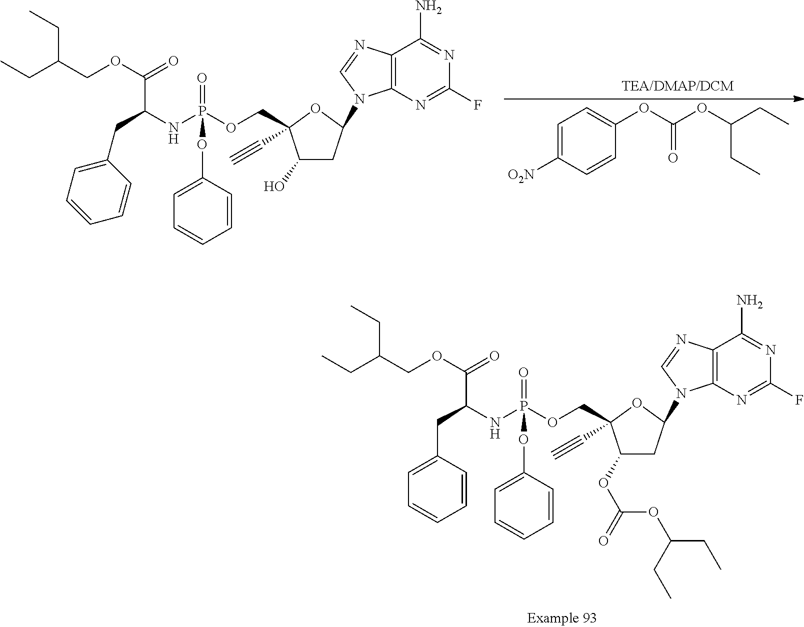 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCC(CC)OC(=O)Oc1ccc([N+](=O)[O-])cc1 ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OC(CC)CC]

### 2-Ethylbutyl ((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)-L-phenylalaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.294 mmol) in DCM (10 mL) was treated with triethylamine (0.123 mL, 0.881 mmol), DMAP (35.9 mg, 0.294 mmol) followed by 4-nitrophenyl pentan-3-yl carbonate (298 mg, 1.175 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford 2-ethylbutyl ((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)-L-phenylalaninate (110 mg, 0.138 mmol, 47.1% yield) as white solid. LCMS (M+H)=795.3; Retention time (0.1% TFA)=1.958 min. ¹H NMR (400 MHz, CDCl₃) δ 7.96 (s, 1H), 7.26-7.18 (m, 5H), 7.14 (dd, J=14.1, 8.3 Hz, 5H), 6.40 (s, 1H), 6.11-5.61 (m, 2H), 5.47 (d, J=2.9 Hz, 1H), 4.63 (d, J=6.2 Hz, 1H), 4.24 (dd, J=29.4, 6.0 Hz, 3H), 3.95 (dd, J=5.8, 2.9 Hz, 2H), 3.63 (s, 1H), 3.01 (d, J=6.5 Hz, 2H), 2.69 (d, J=33.6 Hz, 3H), 1.68 (s, 1H), 1.66-1.56 (m, 3H), 1.50-1.35 (m, 1H), 1.33-1.15 (m, 4H), 0.95 (ddd, J=9.8, 5.6, 1.9 Hz, 6H), 0.83 (t, J=7.4 Hz, 6H).

### Example 94

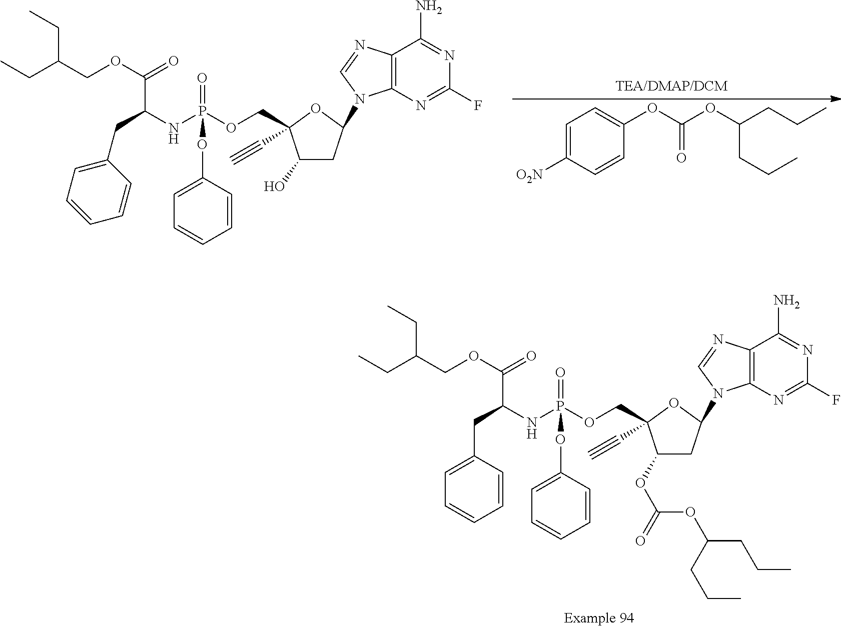 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCC(CCC)OC(=O)Oc1ccc([N+](=O)[O-])cc1 ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OC(CCC)CCC]

### 2-Ethylbutyl ((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)-L-phenylalaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.294 mmol) in DCM (10 mL) was treated with triethylamine (0.123 mL, 0.881 mmol), DMAP (35.9 mg, 0.294 mmol) followed by heptan-4-yl (4-nitrophenyl) carbonate (331 mg, 1.175 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford 2-ethylbutyl ((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)-L-phenylalaninate (99 mg, 0.118 mmol, 40.1% yield) as white solid. LCMS (M+H)=823.3; Retention time (0.05% TFA)=2.056 min; purity (254 nm)=98%. ¹H NMR (400 MHz, CDCl₃) δ 7.96 (s, 1H), 7.31-6.99 (m, 11H), 6.59 (s, 2H), 6.39 (s, 1H), 5.46 (d, J=2.8 Hz, 1H), 4.78 (s, 1H), 4.30 (dd, J=10.8, 6.5 Hz, 2H), 4.22 (s, 1H), 4.04 (s, 1H), 3.95 (d, J=5.4 Hz, 2H), 3.00 (d, J=5.6 Hz, 2H), 2.68 (d, J=30.9 Hz, 3H), 1.72-1.51 (m, 4H), 1.33 (ddd, J=21.0, 13.5, 6.8 Hz, 9H), 1.04-0.71 (m, 12H).

### Example 95

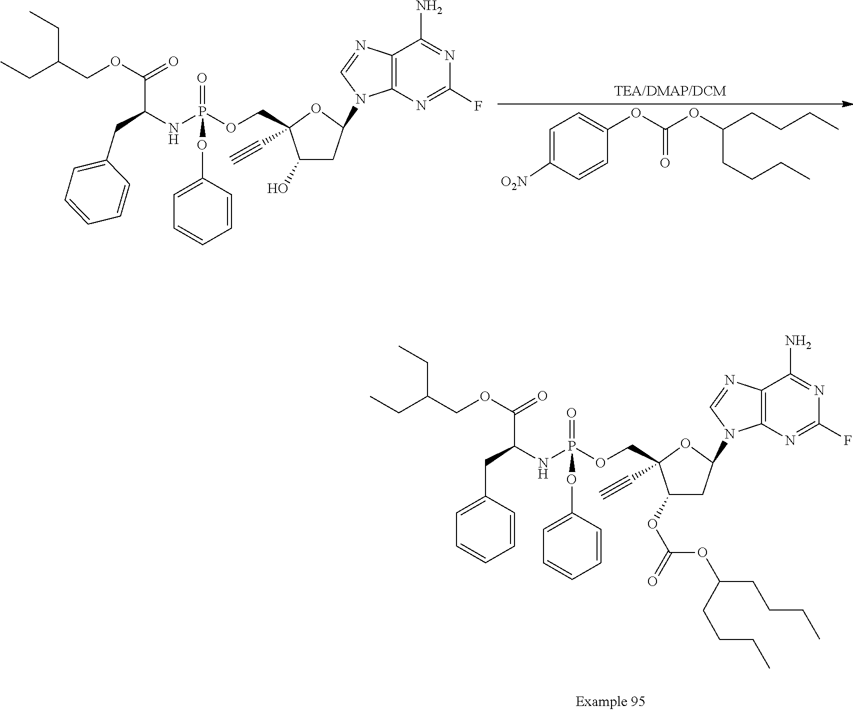 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCCC(CCCC)OC(=O)Oc1ccc([N+](=O)[O-])cc1 ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OC(CCCC)CCCC]

### 2-Ethylbutyl ((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)-L-phenylalaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.294 mmol) in DCM (10 mL) was treated with triethylamine (0.123 mL, 0.881 mmol), DMAP (35.9 mg, 0.294 mmol) followed by 4-nitrophenyl nonan-5-yl carbonate (364 mg, 1.175 mmol) and the mixture was stirred at RT for 2 days. TLC showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford 2-ethylbutyl ((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)-L-phenylalaninate (119 mg, 0.140 mmol, 47.6% yield) as white solid. LCMS (M+H)=851.3; Retention time (0.05% TFA)=2.178 min. ¹H NMR (400 MHz, CDCl₃) δ 7.96 (s, 1H), 7.26-7.17 (m, 5H), 7.17-7.06 (m, 5H), 6.58 (s, 2H), 6.39 (s, 1H), 5.46 (s, 1H), 4.82-4.67 (m, 1H), 4.44-4.14 (m, 3H), 4.10-3.85 (m, 3H), 3.10-2.93 (m, 2H), 2.63 (s, 3H), 1.76-1.51 (m, 4H), 1.48-1.13 (m, 13H), 1.04-0.69 (m, 12H).

### Example 96

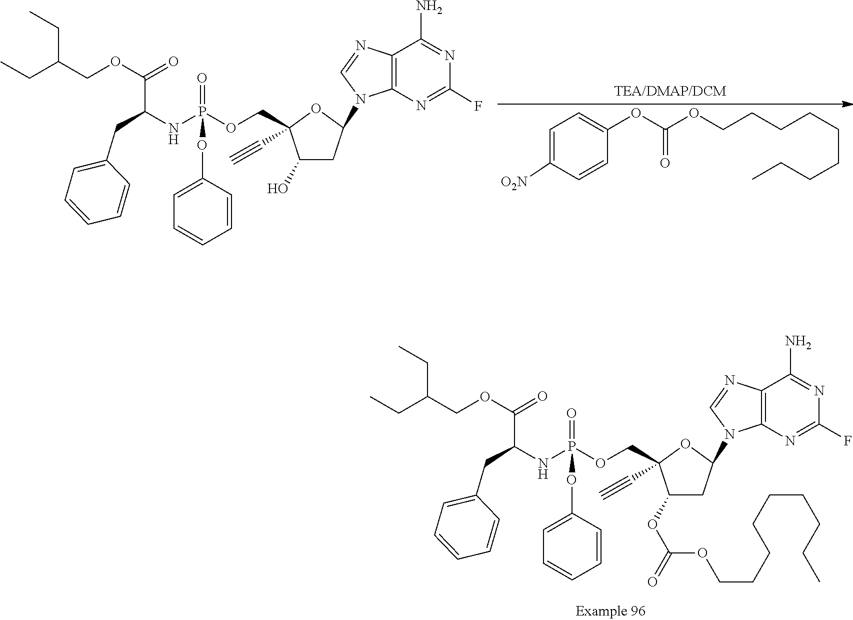 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCCCCCCCOC(=O)Oc1ccc([N+](=O)[O-])cc1 ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCCCC]

### 2-Ethylbutyl ((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)-L-phenylalaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.294 mmol) in DCM (10 mL) was treated with triethylamine (0.123 mL, 0.881 mmol), DMAP (35.9 mg, 0.294 mmol) followed by 4-nitrophenyl nonyl carbonate (364 mg, 1.175 mmol) and the mixture was stirred at RT for 2 days. LCMS showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford 2-ethylbutyl ((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)-L-phenylalaninate (148 mg, 0.172 mmol, 58.6% yield) as white solid. LCMS (M+H)=851.4; Retention time (0.05% TFA)=2.229 min. ¹H NMR (400 MHz, CDCl₃) δ 7.94 (s, 1H), 7.26-7.18 (m, 5H), 7.17-7.03 (m, 5H), 6.38 (s, 1H), 5.96 (s, 2H), 5.48 (d, J=2.5 Hz, 1H), 4.27 (d, J=6.3 Hz, 2H), 4.25-4.07 (m, 3H), 3.95 (dd, J=5.8, 2.4 Hz, 2H), 3.67 (s, 1H), 3.09-2.92 (m, 2H), 2.71 (d, J=34.1 Hz, 3H), 1.69 (s, 1H), 1.53-1.07 (m, 18H), 0.86 (dt, J=14.9, 7.1 Hz, 9H).

### Example 97

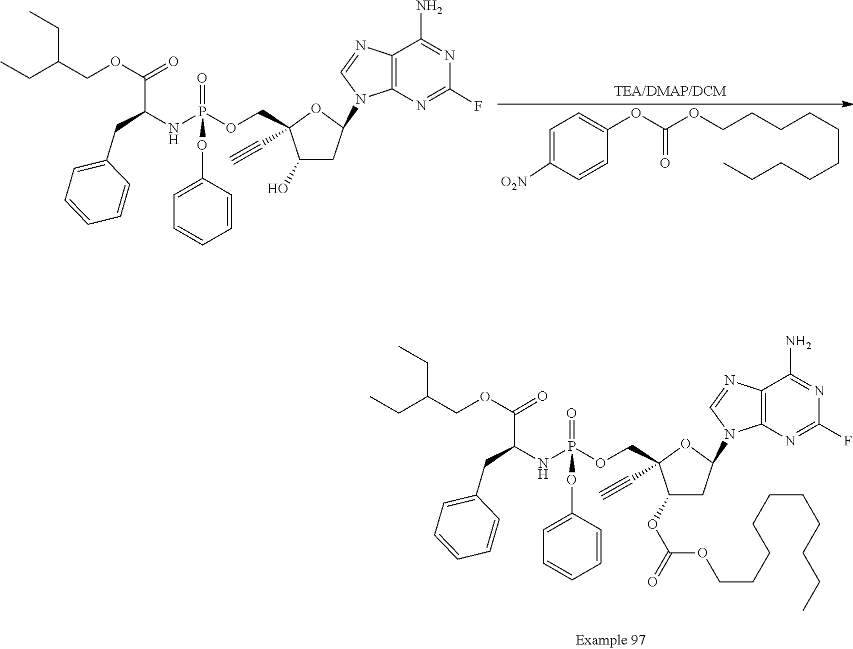 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1O ; CCCCCCCCCCOC(=O)Oc1ccc([N+](=O)[O-])cc1 ; C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCCCCC]

### 2-Ethylbutyl ((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)-L-phenylalaninate

A mixture of 2-ethylbutyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.294 mmol) in DCM (10 mL) was treated with triethylamine (0.123 mL, 0.881 mmol), DMAP (35.9 mg, 0.294 mmol) followed by decyl (4-nitrophenyl) carbonate (380 mg, 1.175 mmol) and the mixture was stirred at RT for 2 days. LCMS showed the reaction was completed. The reaction mixture was concentrated in vacuum and the residue was purified by reverse phase chromatography (SepaFlash@ C18 column, BOSTON, 40 g, 0-100% CH3CN/10 mM aq. NH4HCO3) to afford 2-ethylbutyl ((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)-L-phenylalaninate (152 mg, 0.174 mmol, 59.2% yield) as white solid. LCMS (M+H)=865.4; Retention time (0.05% TFA)=2.327 min. ¹H NMR (400 MHz, CDCl₃) δ 7.95 (s, 1H), 7.22 (dd, J=12.7, 7.1 Hz, 5H), 7.13 (dd, J=14.8, 7.6 Hz, 5H), 6.38 (t, J=6.7 Hz, 1H), 5.94-5.67 (m, 2H), 5.47 (d, J=7.0 Hz, 1H), 4.43-4.23 (m, 2H), 4.17 (dt, J=11.1, 6.1 Hz, 3H), 3.95 (dd, J=5.8, 2.8 Hz, 2H), 3.59 (s, 1H), 3.01 (s, 2H), 2.86-2.54 (m, 3H), 1.70 (s, 1H), 1.34 (d, J=52.8 Hz, 20H), 0.86 (dt, J=14.9, 7.1 Hz, 9H).

### Example 98

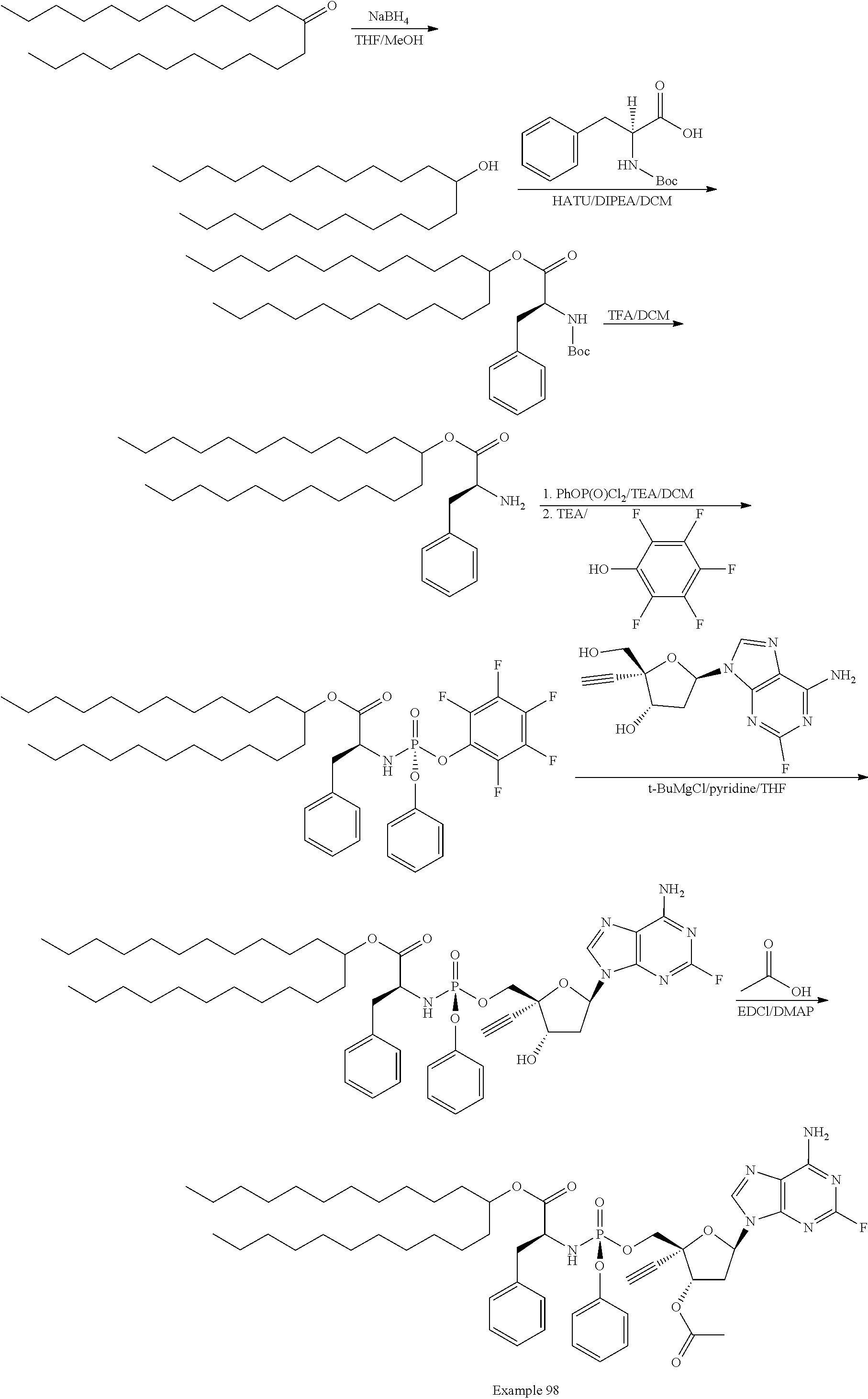

### Step 1: Tricosan-12-ol

To a solution of tricosan-12-one (100 g, 295 mmol) in THF (600 mL) and methanol (100 mL) was added NaBH₄ (16.76 g, 443 mmol) at 15° C. The reaction mixture was stirred at rt for 16 h. TLC showed the reaction was completed. The reaction mixture was quenched with NH₄Cl aqueous solution and concentrated to remove organic solvent. Water (1500 mL) was added and solid formed was filtered and washed with water (800 mL). The cake was dried under reduced pressure to give tricosan-12-ol (95 g, 279 mmol, 94% yield) as a white solid which was used in the next step without further purification. ¹H NMR (400 MHz, MeOD) δ 3.50 (s, 1H), 1.42 (s, 4H), 1.30 (s, 36H), 0.90 (s, 6H).

### Step 2: Tricosan-12-yl (tert-butoxycarbonyl)-L-phenylalaninate

To a mixture of (tert-butoxycarbonyl)-L-phenylalanine (24.53 g, 92 mmol), 1H-imidazole (17.99 g, 264 mmol) and HATU (50.2 g, 132 mmol) in DCM (500 mL) was added DIPEA (46.1 mL, 264 mmol). After stirring for 30 min, tricosan-12-ol (30 g, 88 mmol) was added and the mixture was stirred at rt for 16 h. TLC (pet. ether:EtOAc=40:1, Rf=0.4) showed the reaction was completed. The mixture was washed with water, the organic layer dried over Na₂SO₄, filtered and concentrated in vacuum. The crude product was dissolved in DCM, loaded to silica column (SanTai, 330 g), and purified by flash (Biotage) chromatography with pet. ether (100 mL/min) to give tricosan-12-yl (tert-butoxycarbonyl)-L-phenylalaninate (43 g, 73.1 mmol, 83% yield) as a yellow oil. ¹H NMR (400 MHz, CDCl₃) δ 7.29 (d, J=6.8 Hz, 1H), 7.26-7.19 (m, 2H), 7.18-7.14 (m, 2H), 4.96 (d, J=8.2 Hz, 1H), 4.89-4.82 (m, 1H), 4.55 (dd, J=14.1, 6.4 Hz, 1H), 3.13 (dd, J=13.8, 6.0 Hz, 1H), 3.01 (dd, J=13.8, 6.4 Hz, 1H), 1.47 (d, J=17.4 Hz, 4H), 1.41 (s, 9H), 1.25 (s, 36H), 0.88 (t, J=6.7 Hz, 6H).

### Step 3: Tricosan-12-yl L-phenylalaninate

To a solution of tricosan-12-yl (tert-butoxycarbonyl)-L-phenylalaninate (50 g, 85 mmol) in DCM (100 mL) was added TFA (45.9 mL, 595 mmol) at 0° C. The mixture was stirred at rt for 16 h. TLC (pet. ether:EtOAc=5:1, Rf=0.4) showed the reaction was completed. The reaction was concentrated and pH was adjusted to pH=8-9 with sat. NaHCO₃. The mixture was extracted with DCM (250 mL×2). The combined organic phases were washed with sat. NaHCO₃ (100 mL), dried over Na₂SO₄, filtered and concentrated. The crude product was dissolved in DCM, loaded to silica column (SanTai, 330 g), and purified by flash (Biotage) chromatography (0-7% MeOH/DCM, 100 mL/min) to give tricosan-12-yl L-phenylalaninate (38 g, 78 mmol, 92% yield) as a yellow oil. LCMS (M+H)+=488.5, Retention time (0.1% TFA)=2.907 min; ¹H NMR (400 MHz, CDCl₃) δ 7.32-7.27 (m, 2H), 7.23 (dd, J=9.9, 4.5 Hz, 3H), 4.89 (quint, J=6.2 Hz, 1H), 3.70 (dd, J=8.2, 5.3 Hz, 1H), 3.11 (dd, J=13.6, 5.3 Hz, 1H), 2.81 (dd, J=13.5, 8.2 Hz, 1H), 1.51 (d, J=5.1 Hz, 4H), 1.31-1.29 (m, 36H), 0.88 (t, J=6.7 Hz, 6H).

### Step 4: Tricosan-12-yl ((S)-(perfluorophenoxy)(phenoxy)phosphoryl)-L-phenylalaninate

To a solution of tricosan-12-yl L-phenylalaninate (35 g, 71.7 mmol) in anhydrous DCM (550 mL) was added dropwise triethylamine (10.47 mL, 75 mmol) over 20 min at −70° C. To this mixture was added a solution of phenyl phosphorodichloridate (14.99 g, 71.0 mmol) in anhydrous DCM (50 mL) over 1 h. The reaction mixture was stirred at this temperature for additional 30 min, allowed to warm to 0° C. over 2 h and stirred for additional 1 h. To this mixture was added a solution of 2,3,4,5,6-pentafluorophenol (13.07 g, 71.0 mmol) and triethylamine (10.97 mL, 79 mmol) in dichloromethane (70 mL) over 20 min and stirred at 0° C. for 3 h. TLC (pet. ether:EtOAc=5:1, Rf=0.5) showed the reaction was completed. The reaction mixture was concentrated under reduced pressure. The residue was triturated with TBME (700 mL) and the triethylaminehydrochloride salt was removed by filtration. The filter cake was washed with TBME (2×100 mL) and the combined filtrate was concentrated under reduced pressure to give a crude product as a mixture of diastereomers. The mixture was triturated with pet. ether (150 mL) and solids collected by filtration to give tricosan-12-yl ((S)-(perfluorophenoxy)(phenoxy)phosphoryl)-L-phenylalaninate (17 g, 20.99 mmol, 29.3% yield) (>98% de as determined by ³¹P NMR) as a white solid. ¹H NMR (400 MHz, CDCl₃) δ 7.33 (t, J=7.8 Hz, 2H), 7.25-7.21 (m, 3H), 7.19 (t, J=8.4 Hz, 3H), 7.10 (dd, J=7.2, 2.0 Hz, 2H), 4.84 (quint, J=6.1 Hz, 1H), 4.47-4.37 (m, 1H), 3.88-3.80 (m, 1H), 3.10 (d, J=6.0 Hz, 2H), 1.50-1.42 (m, 4H), 1.33-1.28 (m, 4H), 1.23 (d, J=14.9 Hz, 32H), 0.88 (t, J=6.7 Hz, 6H). ³¹P NMR (162 MHz, CDCl₃) δ−1.54 (s).

### Step 5: Tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a solution of (2R,3S,5R)-5-(6-amino-2-fluoro-9H-purin-9-yl)-2-ethynyl-2-(hydroxymethyl)tetrahydrofuran-3-ol (0.6 g, 2.4 mmol) in THF (120 mL) and pyridine (30 mL) was added dropwise tert-butylmagnesium chloride (4.3 mL, 4.3 mmol) at −15° C. After stirring for 30 min, a solution of tricosan-12-yl ((S)-(perfluorophenoxy)(phenoxy)phosphoryl)-L-phenylalaninate (2.16 g, 2.66 mmol) in THF (15 mL) was added dropwise to the above solution at −15° C. and the reaction was stirred at −15° C. for 4 h. This reaction was conducted 5 batches (600 mg scale, total 3 g (2R,3S,5R)-5-(6-amino-2-fluoro-9H-purin-9-yl)-2-ethynyl-2-(hydroxymethyl)tetrahydrofuran-3-ol used). LCMS showed the reaction was completed. The reaction was quenched with sat. NH₄Cl (20 mL) and concentrated. The crude was extracted with EtOAc (80 mL×3). The combined organic layers were washed with brine (80 mL), dried with Na₂SO₄, filtered and concentrated in vacuu. The crude product was dissolved in THF and purified by reverse flash chromatography (Biotage, gradient: 70%-100% CAN/10 mM aq. NH₄HCO₃, flow rate: 60 mL/min, column: Boston C18, 80 g) to give tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (0.45 g, 0.490 mmol, 23.93% yield) (Total amount: 2.7 g) as a light yellow solid. LCMS (M+H)+=919.5, Retention time (0.1% TFA)=4.371 min.

### Step 6: Tricosan-12-yl ((S)-(((2R,3S,5R)-3-acetoxy-5-(6-amino-2-fluoro-9H-purin-9-yl)-2-ethynyltetrahydrofuran-2-yl)methoxy)(phenoxy)phosphoryl)-L-phenylalaninate

To a solution of acetic acid (19.60 mg, 0.326 mmol) in DCM (6 mL) was added DMAP (26.6 mg, 0.218 mmol) and EDC (83 mg, 0.435 mmol) and the resulting mixture was stirred for 0.5 h at ice-water bath temperature. Then, tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (200 mg, 0.218 mmol) was added and the resulting mixture was stirred at 25° C. for 16 h. The LCMS and TLC showed the reaction was completed. The reaction mixture was concentrated in vacuum and the residue was dissolved in DCM (2 mL). Then, the solution was loaded to silica gel column (SanTai, 25 g) and purified by flash (Biotage) column chromatography (0-5% MeOH in DCM; flow rate: 30 mL/min) to give tricosan-12-yl ((S)-(((2R,3S,5R)-3-acetoxy-5-(6-amino-2-fluoro-9H-purin-9-yl)-2-ethynyltetrahydrofuran-2-yl)methoxy)(phenoxy)phosphoryl)-L-phenylalaninate (141 mg, 0.147 mmol, 67.4% yield) as a colorless oil. LCMS (M+H)=961.4; Retention time (0.05% TFA)=2.66 min. ¹H NMR (400 MHz, CDCl₃) δ 8.11 (s, 1H), 7.23 (d, J=7.2 Hz, 3H), 7.21-7.12 (m, 5H), 7.09 (t, J=7.2 Hz, 1H), 6.39 (t, J=6.8 Hz, 1H), 6.26 (brs, 2H), 5.54 (dd, J=6.4, 3.6 Hz, 1H), 4.82 (quint, J=6.0 Hz, 1H), 4.39-4.29 (m, 1H), 4.17 (dd, J=11.2, 6.8 Hz, 1H), 4.04 (dd, J=11.2, 6.0 Hz, 1H), 3.68 (t, J=10.4 Hz, 1H), 3.09 (dd, J=13.6, 6.0 Hz, 1H), 2.95 (dd, J=13.6, 7.2 Hz, 1H), 2.65 (s, 1H), 2.61-2.48 (m, 2H), 2.16 (s, 3H), 2.05 (s, 1H), 1.50-1.41 (m, 4H), 1.29-1.14 (m, 36H), 0.87 (dd, J=7.2, 6.0 Hz, 6H).

### Example 99

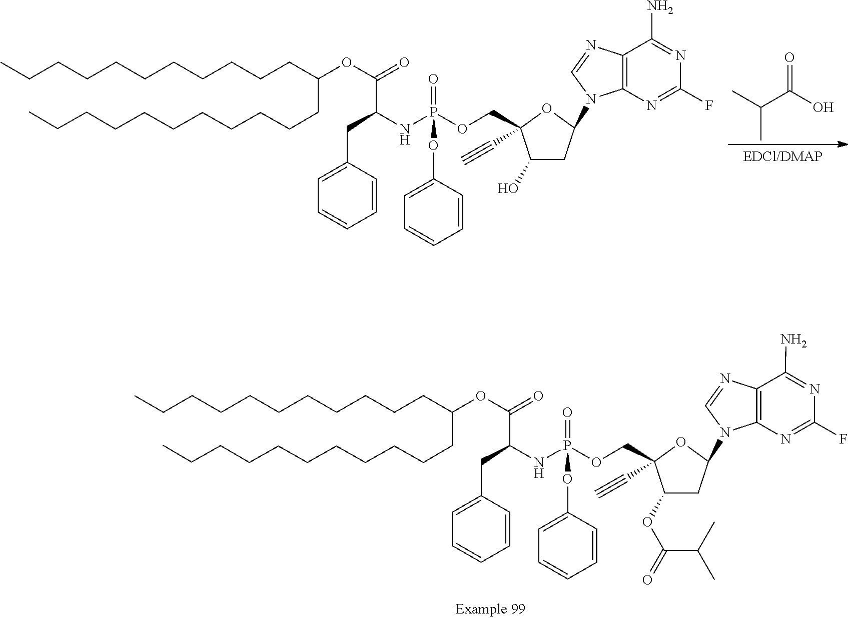

### Tricosan-12-yl ((S)-(((2R,3S,5R)-5-(6-amino-2-fluoro-9H-purin-9-yl)-2-ethynyl-3-(isobutyryloxy)tetrahydrofuran-2-yl)methoxy)(phenoxy)phosphoryl)-L-phenylalaninate

A mixture of isobutyric acid (18.69 mg, 0.212 mmol) and DMAP (19.94 mg, 0.163 mmol) in DCM (5 mL) was treated with EDC (62.6 mg, 0.326 mmol). After stirring for 0.5 h, tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.163 mmol) was added and the mixture was stirred at 25° C. for 16 h. TLC showed the reaction was finished. The reaction mixture was concentrated and the crude product was dissolved in DCM (2 mL), loaded to silica column (SanTai, 12 g) and purified by flash (Biotage) column chromatography (0% to 3% MeOH/DCM, 30 mL/min) to give tricosan-12-yl ((S)-(((2R,3S,5R)-5-(6-amino-2-fluoro-9H-purin-9-yl)-2-ethynyl-3-(isobutyryloxy)tetrahydrofuran-2-yl)methoxy)(phenoxy)phosphoryl)-L-phenylalaninate (147 mg, 0.145 mmol, 89% yield) as light yellow oil. HPLC Retention time (10 mM NH₄HCO₃)=18.222 min, purity: 97.30%. ¹H NMR (400 MHz, CDCl₃) δ 8.07 (s, 1H), 7.23 (d, J=7.7 Hz, 4H), 7.16 (dd, J=17.2, 7.1 Hz, 5H), 7.09 (t, J=7.3 Hz, 1H), 6.38 (t, J=6.7 Hz, 1H), 6.3-6.1 (m, 1H), 5.51 (dd, J=6.7, 4.1 Hz, 1H), 4.81 (p, J=6.0 Hz, 1H), 4.39-4.28 (m, 1H), 4.17 (dd, J=11.1, 6.5 Hz, 1H), 4.05 (dd, J=11.1, 6.0 Hz, 1H), 3.66 (t, J=10.5 Hz, 1H), 3.09 (dd, J=14.2, 5.6 Hz, 1H), 2.94 (dd, J=13.9, 7.1 Hz, 1H), 2.69-2.60 (m, 2H), 2.59-2.49 (m, 2H), 1.44 (s, 4H), 1.28-1.16 (m, 42H), 0.87 (dt, J=7.0, 3.3 Hz, 6H).

### Example 100

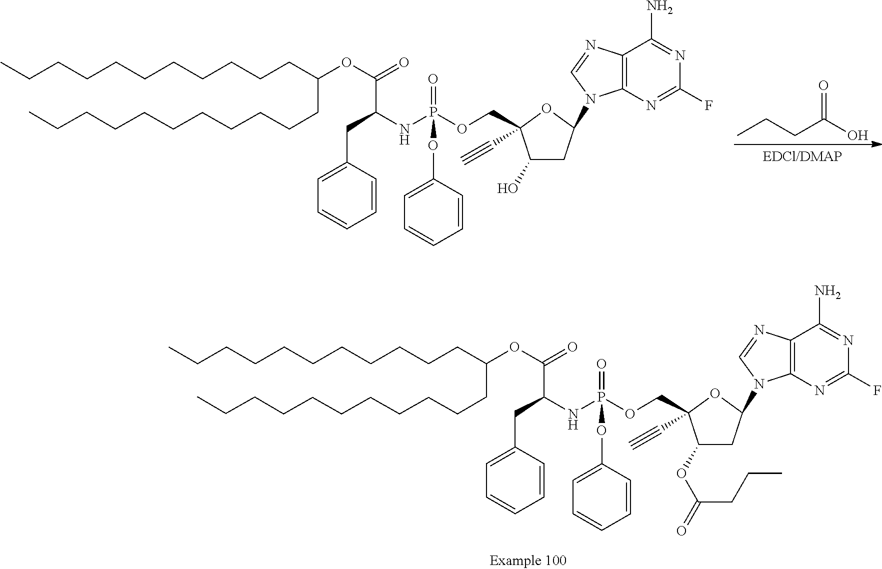

### (2R,3S,5R)-5-(6-Amino-2-fluoro-9H-purin-9-yl)-2-ethynyl-2-((((S)-(((S)-1-oxo-3-phenyl-1-(tricosan-12-yloxy)propan-2-yl)amino)(phenoxy)phosphoryl)oxy)methyl)tetrahydrofuran-3-yl butyrate

A mixture of butyric acid (18.69 mg, 0.212 mmol) and DMAP (19.94 mg, 0.163 mmol) in DCM (5 mL) was treated with EDC (62.6 mg, 0.326 mmol). After stirring for 0.5 h, tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.163 mmol) was added and the mixture was stirred at 25° C. for 16 h. LCMS showed the reaction was finished. The reaction mixture was concentrated and the crude product was dissolved in DCM (2 mL), loaded to silica column (SanTai, 12 g) and purified by flash (Biotage) column chromatography (0% to 3% MeOH/DCM, 30 mL/min) to give (2R,3S,5R)-5-(6-amino-2-fluoro-9H-purin-9-yl)-2-ethynyl-2-((((S)-(((S)-1-oxo-3-phenyl-1-(tricosan-12-yloxy)propan-2-yl)amino)(phenoxy)phosphoryl)oxy)methyl)tetrahydrofuran-3-yl butyrate (101 mg, 0.098 mmol, 60.3% yield) as light yellow oil. HPLC Retention time (10 mM NH₄HCO₃)=18.423 min, purity: 96.39%. ¹H NMR (400 MHz, CDCl₃) δ 8.09 (s, 1H), 7.26-7.21 (m, 4H), 7.20-7.12 (m, 5H), 7.09 (t, J=7.2 Hz, 1H), 6.40-6.10 (m, 2H), 5.54 (dd, J=6.7, 4.0 Hz, 1H), 4.81 (dd, J=12.3, 6.1 Hz, 1H), 4.38-4.27 (m, 1H), 4.17 (dd, J=11.1, 6.6 Hz, 1H), 4.05 (dd, J=11.1, 6.0 Hz, 1H), 3.65 (t, J=10.5 Hz, 1H), 3.09 (dd, J=13.9, 5.9 Hz, 1H), 2.94 (dd, J=13.8, 7.1 Hz, 1H), 2.63-2.61 (m, 1H), 2.60-2.46 (m, 2H), 2.39 (t, J=7.4 Hz, 2H), 1.75-1.67 (m, 2H), 1.44 (s, 4H), 1.26 (d, J=17.2 Hz, 36H), 1.02-0.97 (m, 3H), 0.87 (dd, J=6.9, 5.6 Hz, 6H).

### Example 101

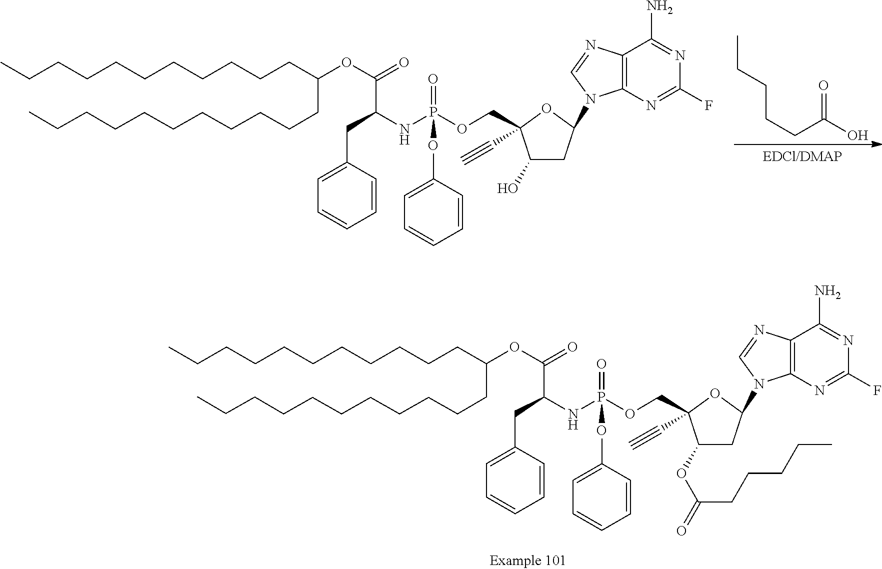

### (2R,3S,5R)-5-(6-Amino-2-fluoro-9H-purin-9-yl)-2-ethynyl-2-((((S)-(((S)-1-oxo-3-phenyl-1-(tricosan-12-yloxy)propan-2-yl)amino)(phenoxy)phosphoryl)oxy)methyl)tetrahydrofuran-3-yl hexanoate

To a solution of hexanoic acid (24.64 mg, 0.212 mmol) in DCM (6 mL) were added DMAP (19.94 mg, 0.163 mmol) and EDC (62.6 mg, 0.326 mmol) and the resulting mixture was stirred for 0.5 h at ice-water bath temperature. Then, tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.163 mmol) was added and the resulting mixture was stirred at 25° C. for 16 h. The LCMS and TLC showed the reaction was completed. The reaction mixture was concentrated in vacuum and the residue was dissolved in DCM (2 mL). Then, the mixture was loaded to silica gel column (SanTai, 25 g) and purified by flash (Biotage) column chromatography (0-5% MeOH in DCM; flow rate: 30 mL/min) to give (2R,3S,5R)-5-(6-amino-2-fluoro-9H-purin-9-yl)-2-ethynyl-2-((((S)-(((S)-1-oxo-3-phenyl-1-(tricosan-12-yloxy)propan-2-yl)amino)(phenoxy)phosphoryl)oxy)methyl)tetrahydrofuran-3-yl hexanoate (127 mg, 0.125 mmol, 76% yield) as a colorless oil. HPLC: Retention time (10 mM NH₄HCO₃)=23.78 min, purity: 100%. ¹H NMR (400 MHz, CDCl₃) δ 8.02 (s, 1H), 7.23 (d, J=7.2 Hz, 3H), 7.21-7.16 (m, 3H), 7.14 (d, J=8.4 Hz, 2H), 7.09 (t, J=7.6 Hz, 1H), 6.38 (t, J=6.8 Hz, 1H), 5.53 (dd, J=6.4, 3.6 Hz, 1H), 4.82 (quint, J=6.4 Hz, 1H), 4.37-4.29 (m, 1H), 4.16 (dd, J=11.6, 7.2 Hz, 1H), 4.05 (dd, J=11.2, 6.0 Hz, 1H), 3.63 (t, J=10.4 Hz, 1H), 3.09 (dd, J=14.0, 5.6 Hz, 1H), 2.94 (dd, J=13.6, 7.2 Hz, 1H), 2.63 (s, 1H), 2.61-2.55 (m, 1H), 2.49 (quint, J=7.2 Hz, 1H), 2.40 (t, J=7.6 Hz, 2H), 1.71-1.65 (m, 2H), 1.37-1.32 (m, 4H), 1.28-1.16 (m, 40H), 0.94-0.90 (m, 3H), 0.87 (dt, J=6.8, 1.2 Hz, 6H).

### Example 102

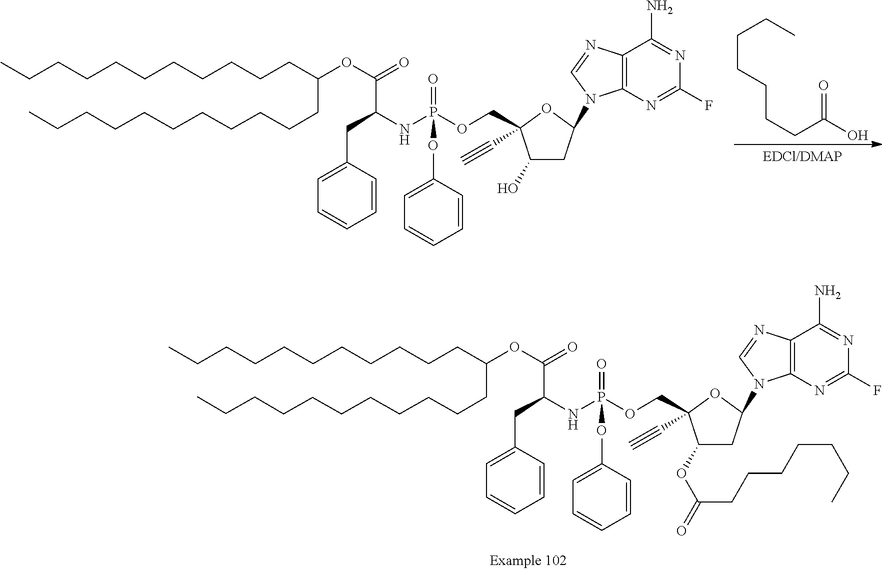

### (2R,3S,5R)-5-(6-Amino-2-fluoro-9H-purin-9-yl)-2-ethynyl-2-((((S)-(((S)-1-oxo-3-phenyl-1-(tricosan-12-yloxy)propan-2-yl)amino)(phenoxy)phosphoryl)oxy)methyl)tetrahydrofuran-3-yl octanoate

A mixture of octanoic acid (30.6 mg, 0.212 mmol) and DMAP (19.94 mg, 0.163 mmol) in DCM (5 mL) was treated with EDC (62.6 mg, 0.326 mmol). After stirring for 0.5 h, tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.163 mmol) was added and the mixture was stirred at 25° C. for 16 h. TLC showed the reaction was finished. The reaction mixture was concentrated and the crude product was dissolved in DCM (2 mL), loaded to silica column (SanTai, 12 g) and purified by flash (Biotage) column chromatography (0% to 3% MeOH/DCM, 30 mL/min) to give (2R,3S,5R)-5-(6-amino-2-fluoro-9H-purin-9-yl)-2-ethynyl-2-((((S)-(((S)-1-oxo-3-phenyl-1-(tricosan-12-yloxy)propan-2-yl)amino)(phenoxy)phosphoryl)oxy)methyl)tetrahydrofuran-3-yl octanoate (160 mg, 0.150 mmol, 92% yield) as light yellow oil. HPLC Retention time (10 mM NH₄HCO₃)=30.92 min. ¹H NMR (400 MHz, CDCl₃) δ 8.13 (s, 1H), 7.23 (d, J=7.4 Hz, 4H), 7.18 (d, J=5.4 Hz, 2H), 7.17-7.11 (m, 3H), 7.09 (t, J=7.3 Hz, 1H), 6.37 (t, J=6.7 Hz, 1H), 5.52 (dd, J=6.9, 3.8 Hz, 1H), 4.82 (p, J=6.3 Hz, 1H), 4.33 (dq, J=9.6, 6.9 Hz, 1H), 4.17 (dd, J=11.2, 6.6 Hz, 1H), 4.05 (dd, J=11.2, 6.0 Hz, 1H), 3.68 (t, J=10.5 Hz, 1H), 3.09 (dd, J=13.7, 5.5 Hz, 1H), 2.94 (dd, J=13.9, 7.3 Hz, 1H), 2.64-2.53 (m, 2H), 2.51-2.44 (m, 1H), 2.39 (dd, J=13.3, 5.6 Hz, 2H), 2.34 (t, J=7.6 Hz, 2H), 1.72-1.59 (m, 4H), 1.34-1.27 (m, 14H), 1.26-1.19 (m, 30H), 0.89 (s, 3H), 0.88-0.87 (m, 4H), 0.86 (s, 2H).

### Example 103

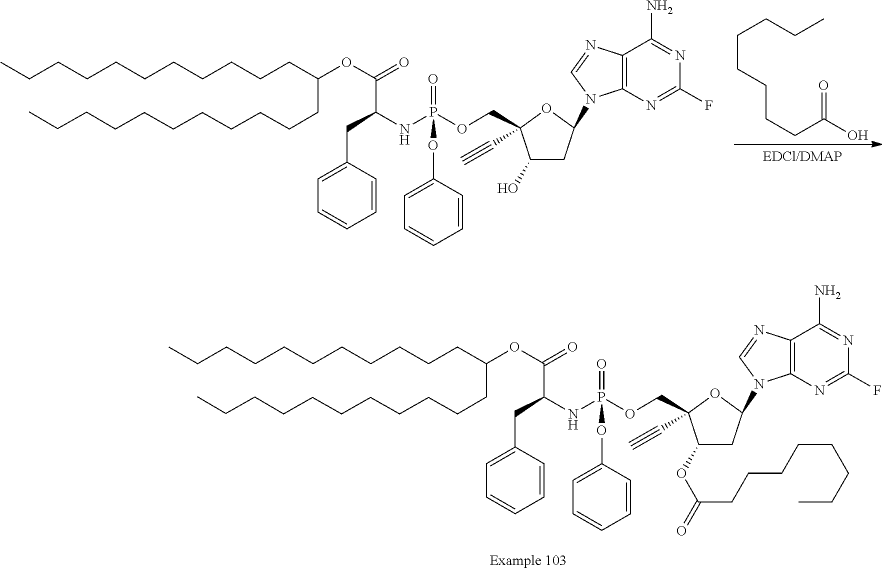

### (2R,3S,5R)-5-(6-Amino-2-fluoro-9H-purin-9-yl)-2-ethynyl-2-((((S)-(((S)-1-oxo-3-phenyl-1-(tricosan-12-yloxy)propan-2-yl)amino)(phenoxy)phosphoryl)oxy)methyl)tetrahydrofuran-3-yl nonanoate

To a solution of nonanoic acid (33.6 mg, 0.212 mmol) in DCM (6 mL) was added DMAP (19.94 mg, 0.163 mmol) and EDC (62.6 mg, 0.326 mmol) and the resulting mixture was stirred for 0.5 h at ice-water bath temperature. Then, tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.163 mmol) was added and the resulting mixture was stirred at 25° C. for 16 h. The LCMS and TLC showed the reaction was completed. The reaction mixture was concentrated in vacuum and the residue was dissolved in DCM (2 mL). Then, the mixture was loaded to silica gel column (SanTai, 25 g) and purified by flash (Biotage) column chromatography (0-5% MeOH in DCM; flow rate: 30 mL/min) to give (2R,3S,5R)-5-(6-amino-2-fluoro-9H-purin-9-yl)-2-ethynyl-2-((((S)-(((S)-1-oxo-3-phenyl-1-(tricosan-12-yloxy)propan-2-yl)amino)(phenoxy)phosphoryl)oxy)methyl)tetrahydrofuran-3-yl nonanoate (136 mg, 0.128 mmol, 79% yield) as a colorless oil. HPLC: Retention time (10 mM NH₄HCO₃)=38.32 min, purity: 100%. ¹H NMR (400 MHz, CDCl₃) δ 8.18 (s, 1H), 7.23 (d, J=7.2 Hz, 3H), 7.21-7.10 (m, 5H), 7.09 (t, J=7.6 Hz, 1H), 6.38 (t, J=7.2 Hz, 1H), 5.52 (dd, J=6.8, 3.6 Hz, 1H), 4.82 (quint, J=6.4 Hz, 1H), 4.38-4.29 (m, 1H), 4.16 (dd, J=11.2, 6.8 Hz, 1H), 4.05 (dd, J=10.8, 6.0 Hz, 1H), 3.61 (t, J=10.4 Hz, 1H), 3.09 (dd, J=14.0, 5.6 Hz, 1H), 2.94 (dd, J=13.6, 7.2 Hz, 1H), 2.63 (s, 1H), 2.58 (ddd, J=14.0, 6.0, 4.0 Hz, 1H), 2.50 (quint, J=6.8 Hz, 1H), 2.40 (t, J=7.6 Hz, 2H), 1.71-1.63 (m, 2H), 1.50-1.42 (m, 4H), 1.34-1.18 (m, 46H), 0.90-0.85 (m, 9H).

### Example 104

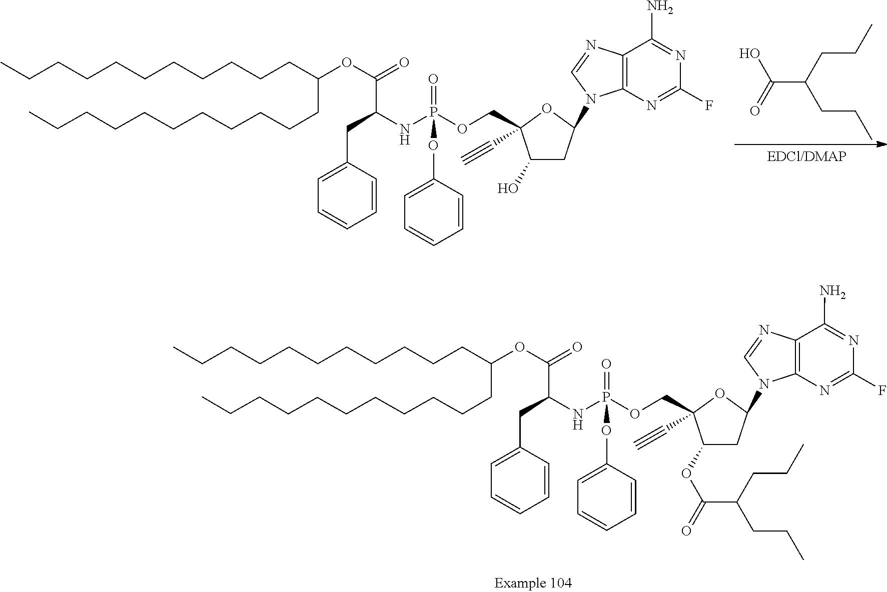

### (2R,3S,5R)-5-(6-Amino-2-fluoro-9H-purin-9-yl)-2-ethynyl-2-((((S)-(((S)-1-oxo-3-phenyl-1-(tricosan-12-yloxy)propan-2-yl)amino)(phenoxy)phosphoryl)oxy)methyl)tetrahydrofuran-3-yl 2-propylpentanoate

To a solution of 2-propylpentanoic acid (30.6 mg, 0.212 mmol) in DCM (6 mL) was added DMAP (19.94 mg, 0.163 mmol) and EDC (62.6 mg, 0.326 mmol) and the resulting mixture was stirred for 0.5 h at ice-water bath temperature. Then, tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (150 mg, 0.163 mmol) was added and the resulting mixture was stirred at 25° C. for 16 h. The LCMS TLC showed the reaction was completed. The reaction mixture was concentrated in vacuum and the residue was dissolved in DCM (2 mL). Then, the mixture was loaded to silica gel column (SanTai, 25 g) and purified by flash (Biotage) column chromatography (0-5% MeOH in DCM; flow rate: 30 mL/min) to give (2R,3S,5R)-5-(6-amino-2-fluoro-9H-purin-9-yl)-2-ethynyl-2-((((S)-(((S)-1-oxo-3-phenyl-1-(tricosan-12-yloxy)propan-2-yl)amino)(phenoxy)phosphoryl)oxy)methyl)tetrahydrofuran-3-yl 2-propylpentanoate (121 mg, 0.116 mmol, 70.9% yield) as a colorless oil. HPLC: Retention time (10 mM NH₄HCO₃)=29.72 min, purity: 100%. ¹H NMR (400 MHz, CDCl₃) δ 7.98 (s, 1H), 7.22 (d, J=7.6 Hz, 3H), 7.20-7.13 (m, 5H), 7.10 (t, J=7.2 Hz, 1H), 6.38 (t, J=6.4 Hz, 1H), 5.52 (dd, J=6.8, 4.8 Hz, 1H), 4.81 (quint, J=6.4 Hz, 1H), 4.40-4.29 (m, 1H), 4.18 (dd, J=11.2, 6.4 Hz, 1H), 4.05 (dd, J=11.2, 5.6 Hz, 1H), 3.78 (t, J=10.4 Hz, 1H), 3.08 (dd, J=13.6, 5.6 Hz, 1H), 2.94 (dd, J=14.0, 7.2 Hz, 1H), 2.68-2.55 (m, 3H), 2.51-2.43 (m, 1H), 1.72-1.62 (m, 2H), 1.52-1.41 (m, 6H), 1.39-1.30 (m, 6H), 1.27-1.17 (m, 34H), 0.92 (dd, J=7.2, 1.6 Hz, 6H), 0.87 (dd, J=6.8, 6.0 Hz, 6H).

### Example 105

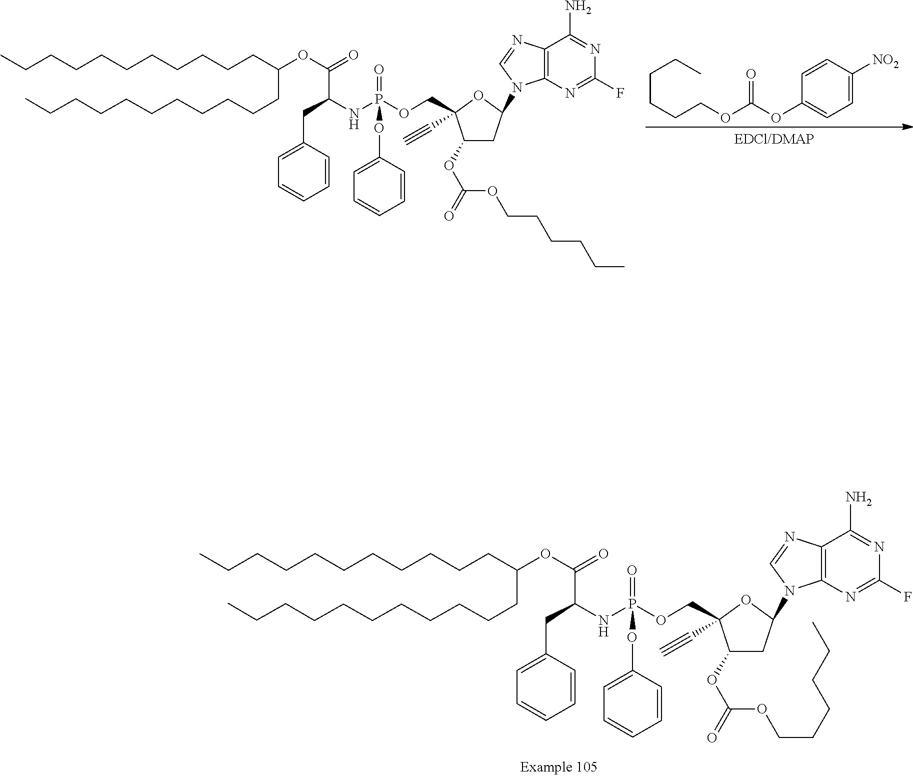

### Tricosan-12-yl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate

A mixture of tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (250 mg, 0.272 mmol) and DMAP (33.2 mg, 0.272 mmol) in DCM (6 mL) was treated with triethylamine (0.114 mL, 0.816 mmol) followed by hexyl (4-nitrophenyl) carbonate (436 mg, 1.632 mmol) and the mixture was stirred at 25° C. for 24 h. The LCMS and TLC showed the reaction was completed. The reaction mixture was concentrated in vacuum and the residue was dissolved in DCM (2 mL). Then, the mixture was loaded to silica gel column (SanTai, 25 g) and purified by flash (Biotage) column chromatography (0-5% MeOH in DCM; flow rate: 30 mL/min) to give tricosan-12-yl ((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)-L-phenylalaninate (148 mg, 0.141 mmol, 52.0% yield) as a white solid. HPLC: Retention time (10 mM NH₄HCO₃)=25.27 min, purity: 100%. ¹H NMR (400 MHz, CDCl₃) δ 7.94 (s, 1H), 7.22 (d, J=8.0 Hz, 3H), 7.21-7.12 (m, 5H), 7.10 (t, J=7.6 Hz, 1H), 6.38 (t, J=6.8 Hz, 1H), 5.99 (brs, 2H), 5.44 (dd, J=6.4, 4.4 Hz, 1H), 4.81 (quint, J=6.0 Hz, 1H), 4.39-4.30 (m, 1H), 4.24-4.17 (m, 3H), 4.08 (dd, J=11.6, 7.2 Hz, 1H), 3.70-3.60 (m, 1H), 3.08 (dd, J=14.0, 6.0 Hz, 1H), 2.94 (dd, J=13.6, 6.8 Hz, 1H), 2.74-2.60 (m, 3H), 1.74-1.67 (m, 4H), 1.42-1.18 (m, 44H), 0.91 (t, J=6.8 Hz, 3H), 0.87 (dt, J=6.8, 1.2 Hz, 6H).

### Example 106

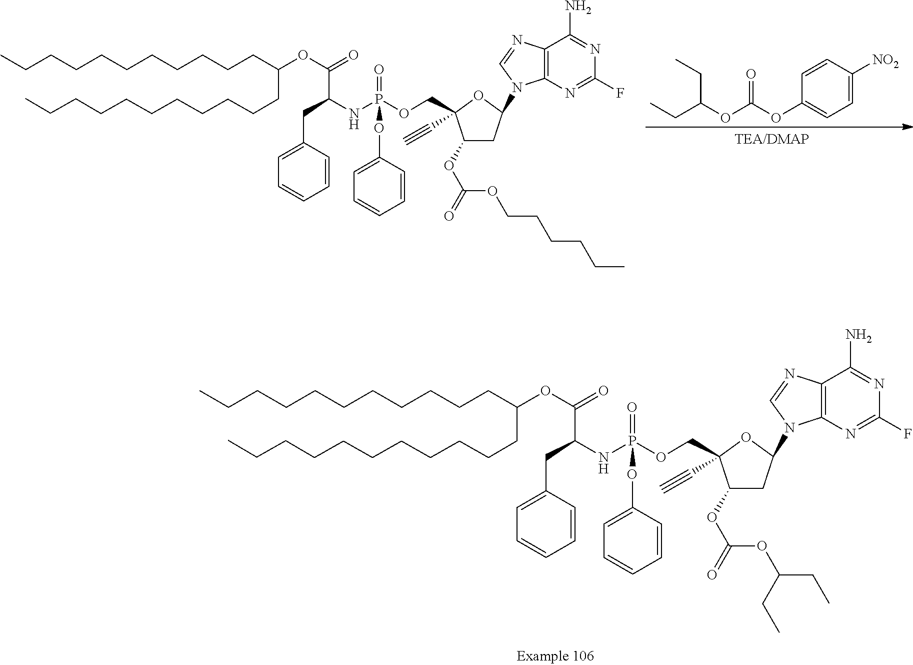

### Tricosan-12-yl ((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)-L-phenylalaninate

A mixture of tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (250 mg, 0.272 mmol) and DMAP (33.2 mg, 0.272 mmol) in DCM (6 mL) was treated with triethylamine (0.114 mL, 0.816 mmol) followed by 4-nitrophenyl pentan-3-yl carbonate (276 mg, 1.088 mmol) and the mixture was stirred at 25° C. for 2 days. The LCMS and TLC showed the reaction was completed. The reaction mixture was concentrated in vacuum and the residue was dissolved in DCM (2 mL). Then, the mixture was loaded to silica gel column (SanTai, 25 g) and purified by flash (Biotage) column chromatography (0-5% MeOH in DCM; flow rate: 30 mL/min) to give tricosan-12-yl ((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)-L-phenylalaninate (133 mg, 0.129 mmol, 47.3% yield) as a colorless oil. HPLC: Retention time (10 mM NH₄HCO₃)=21.24 min, purity: 100%. ¹H NMR (400 MHz, CDCl₃) δ 8.30 (s, 1H), 7.25-7.17 (m, 6H), 7.12 (d, J=8.4 Hz, 2H), 7.08 (t, J=7.6 Hz, 1H), 6.38 (t, J=6.8 Hz, 1H), 5.41 (dd, J=6.8, 3.6 Hz, 1H), 4.83 (quint, J=6.0 Hz, 1H), 4.64 (quint, J=6.0 Hz, 1H), 4.38-4.29 (m, 1H), 4.19 (dd, J=11.6, 7.2 Hz, 1H), 4.12 (dd, J=11.6, 6.8 Hz, 1H), 3.67 (t, J=10.8 Hz, 1H), 3.10 (dd, J=13.2, 6.0 Hz, 1H), 2.95 (dd, J=14.0, 7.2 Hz, 1H), 2.69 (ddd, J=14.4, 6.0, 3.6 Hz, 1H), 2.66 (s, 1H), 2.59-2.50 (m, 1H), 1.72-1.63 (m, 4H), 1.50-1.42 (m, 4H), 1.28-1.14 (m, 36H), 0.95 (dt, J=7.6, 1.6 Hz, 6H), 0.87 (dt, J=7.2, 1.2 Hz, 6H).

### Example 107

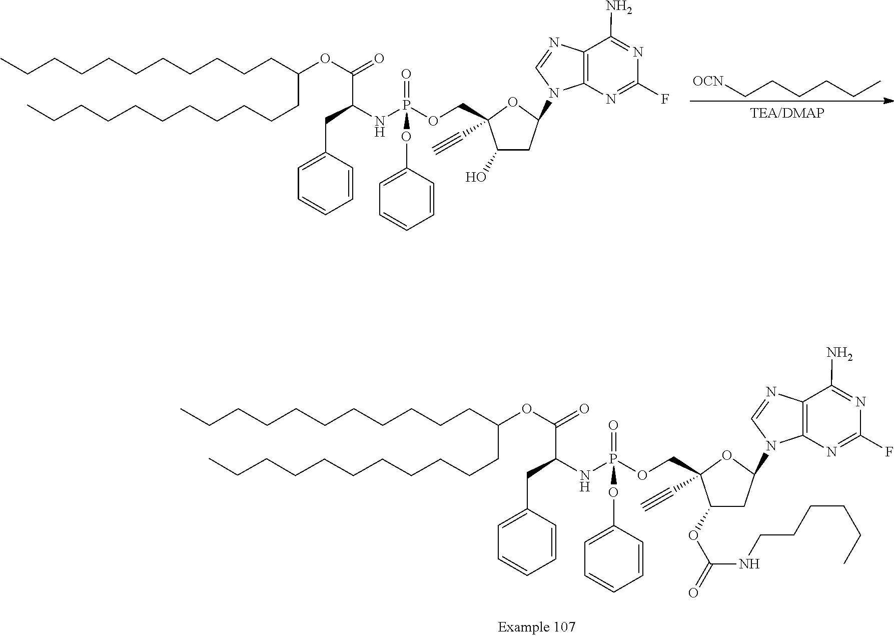

### Tricosan-12-yl ((S)-(((2R,3S,5R)-5-(6-amino-2-fluoro-9H-purin-9-yl)-2-ethynyl-3-((hexylcarbamoyl)oxy)tetrahydrofuran-2-yl)methoxy)(phenoxy)phosphoryl)-L-phenylalaninate

A mixture of tricosan-12-yl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (300 mg, 0.326 mmol) and DMAP (39.9 mg, 0.326 mmol) in DCM (6 mL) was treated with TEA (0.136 mL, 0.979 mmol) followed by 1-isocyanatohexane (0.713 mL, 4.90 mmol) and the mixture was stirred at 40° C. for 7 h. The LCMS and TLC showed the reaction was completed. The reaction mixture was concentrated in vacuum and the residue was dissolved in DCM (2 mL). Then, the mixture was loaded to silica gel column (SanTai, 25 g) and purified by flash (Biotage) column chromatography (0-5% MeOH in DCM; flow rate: 30 mL/min) to give tricosan-12-yl ((S)-(((2R,3S,5R)-5-(6-amino-2-fluoro-9H-purin-9-yl)-2-ethynyl-3-((hexylcarbamoyl)oxy)tetrahydrofuran-2-yl)methoxy)(phenoxy)phosphoryl)-L-phenylalaninate (105 mg, 0.100 mmol, 30.7% yield) as a colorless oil. HPLC: Retention time (10 mM NH₄HCO₃)=23.51 min, purity: 100%. ¹H NMR (400 MHz, CDCl₃) δ 8.30 (s, 1H), 7.25-7.22 (m, 3H), 7.21-7.12 (m, 5H), 7.08 (t, J=7.2 Hz, 1H), 6.35 (t, J=6.8 Hz, 1H), 5.45 (dd, J=7.2, 4.4 Hz, 1H), 4.89-4.79 (m, 2H), 4.28-4.29 (m, 1H), 4.13 (dd, J=11.6, 6.8 Hz, 1H), 4.04 (dd, J=11.2, 6.4 Hz, 1H), 3.71 (t, J=10.4 Hz, 1H), 3.22 (q, J=6.8 Hz, 2H), 3.10 (dd, J=14.0, 5.6 Hz, 1H), 2.94 (dd, J=13.6, 7.2 Hz, 1H), 2.83 (s, 1H), 2.62-2.58 (m, 1H), 2.41 (quint, J=7.2 Hz, 1H), 1.57-1.51 (m, 2H), 1.48-1.41 (m, 4H), 1.33-1.15 (m, 42H), 0.92-0.85 (m, 9H).

### Example 108

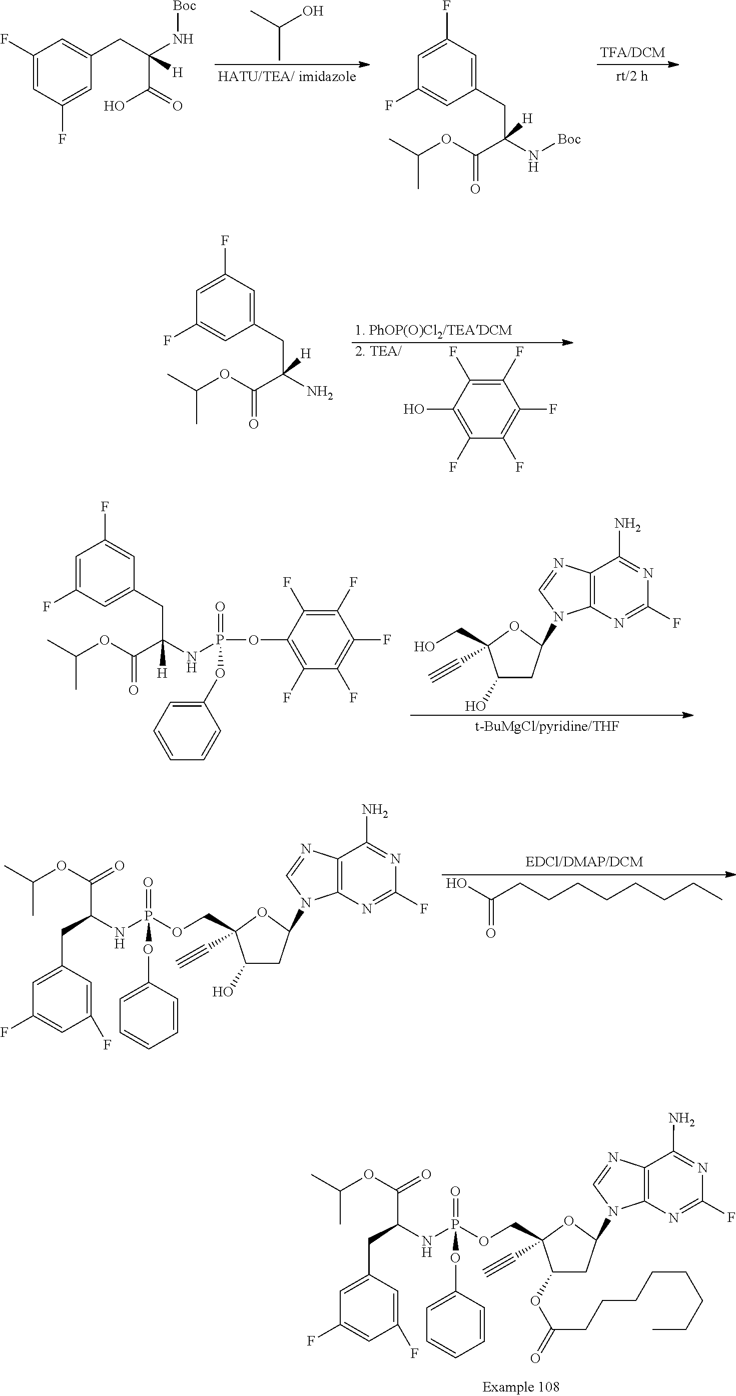

### Step 1: Isopropyl (S)-2-((tert-butoxycarbonyl)amino)-3-(3,5-difluorophenyl)propanoate

To a mixture of propan-2-ol (2.63 g, 43.8 mmol), (S)-2-((tert-butoxycarbonyl)amino)-3-(3,5-difluorophenyl)propanoic acid (12 g, 39.8 mmol) and HATU (18.17 g, 47.8 mmol) in DCM (150 mL) was added 1H-imidazole (8.13 g, 119 mmol) and triethylamine (16.61 mL, 119 mmol). The resulting mixture was stirred at 25° C. overnight. LCMS showed that the reaction was completed. The reaction was diluted with water (60 mL) and DCM (100 mL), then the organic layer was separated. The aqueous layer was extracted with DCM (2×200 mL), then combined organic layers were washed with brine (100 mL), dried over Na₂SO₄, concentrated to give the crude product. The residue was purified by flash column chromatography (Santai, 120 g, hexane:ethyl acetate=10:1). The appropriate fractions containing product were combined, and the solvent was removed in vacuo to give isopropyl (S)-2-((tert-butoxycarbonyl)amino)-3-(3,5-difluorophenyl)propanoate (8 g, 23.30 mmol, 58.5% yield) as yellow oil. LCMS (M+Na)=366.1; Retention time (0.1% TFA)=2.146 min.

### Step 2: Isopropyl (S)-2-amino-3-(3,5-difluorophenyl)propanoate

To a mixture of isopropyl (S)-2-((tert-butoxycarbonyl)amino)-3-(3,5-difluorophenyl)propanoate (22 g, 64.1 mmol) in DCM (100 mL) was added TFA (34.6 mL, 448 mmol) at 0° C., and the resulting reaction mixture was stirred at 5° C. overnight. LCMS showed the reaction was completed. The reaction was concentrated and pH adjusted to 8-9 with sat. NaHCO₃, the mixture was partitioned, and the aqueous phase was extracted with DCM (120 mL×2). The combined organic phases were washed with sat. NaHCO₃ (80 mL), dried over Na₂SO₄ and concentrated. The crude product was dissolved in DCM, loaded to silica column (SanTai, 120 g) and purified by flash (Biotage) column chromatography (0-10% MeOH/DCM, 80 mL/min) to give isopropyl (S)-2-amino-3-(3,5-difluorophenyl)propanoate (12.5 g, 49.9 mmol, 78% yield) as yellow oil. LCMS (M+H)=244.0; Retention time (10 mmol NH₄CO₃)=1.65 min.

### Step 3: Isopropyl (S)-3-(3,5-difluorophenyl)-2-(((S)-(perfluorophenoxy)(phenoxy)phosphoryl)amino)propanoate

To a solution of isopropyl (S)-2-amino-3-(3,5-difluorophenyl)propanoate (12.3 g, 50.6 mmol) in anhydrous DCM (200 mL) was added dropwise triethylamine (7.38 mL, 53.1 mmol) at −70° C. over 10 min. To this mixture was added a solution of phenyl phosphorodichloridate (10.56 g, 50.1 mmol) in anhydrous DCM (50 mL) over 1 h. The reaction mixture was stirred at this temperature for additional 30 min and then allowed to warm to 0° C. over 2 h and stirred for additional 1 h. To this mixture was added a solution of 2,3,4,5,6-pentafluorophenol (9.21 g, 50.1 mmol) and triethylamine (7.73 mL, 55.6 mmol) in DCM (60 mL) over 20 min and the mixture was stirred at 0° C. for 3 h. LCMS showed there was a new spot. The reaction mixture was filtered and filter cake rinsed with DCM. The filtrate was concentrated under reduced pressure and the residue was triturated with TBME (500 mL). The solid triethylaminehydrochloride salt was removed by filtration. The cake was washed with TBME (2×60 mL), and the combined filtrate was concentrated under reduced pressure. The residue was triturated with pet. ether (80 mL) and solids collected by filtration to give isopropyl (S)-3-(3,5-difluorophenyl)-2-(((S)-(perfluorophenoxy)(phenoxy)phosphoryl)amino)propanoate (5.7 g, 10.08 mmol, 19.94% yield) (>98% de as determined by ³¹P NMR) as white solid. LCMS (M+H)=566.0; Retention time (0.1% TFA)=2.264 min. ¹H NMR (400 MHz, CDCl₃) δ 7.36 (t, J=7.9 Hz, 2H), 7.22 (t, J=7.9 Hz, 3H), 7.09 (d, J=6.6 Hz, 1H), 6.67 (tt, J=9.0, 2.2 Hz, 1H), 6.60 (d, J=6.0 Hz, 2H), 5.06-4.94 (m, 1H), 4.42-4.31 (m, 1H), 3.97 (t, J=10.5 Hz, 1H), 3.63-2.74 (m, 6H), 1.29 (d, J=26.7 Hz, 2H), 1.20 (dd, J=11.6, 6.3 Hz, 6H). ³¹P NMR (162 MHz, CDCl₃) δ−1.51 (s).

### Step 4: Isopropyl (S)-2-(((S)-(((2R,3S,5R)-5-(6-Amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a solution of (2R,3S,5R)-5-(6-amino-2-fluoro-9H-purin-9-yl)-2-ethynyl-2-(hydroxymethyl)tetrahydrofuran-3-ol (0.25 g, 0.852 mmol) in THF (40 mL) and pyridine (2 mL) was added dropwise tert-butylmagnesium chloride (1.705 mL, 1.705 mmol) at −15° C. and the reaction mixture was stirred at −15° C. for 1 h. Then, to the reaction mixture was added a solution of isopropyl (S)-3-(3,5-difluorophenyl)-2-(((S)-(perfluorophenoxy)(phenoxy)phosphoryl)amino)propanoate (0.578 g, 1.023 mmol) in THF (15 mL) and stirred at −15° C. for 2 h. The reaction mixture was quenched with NH₄Cl (4 mL) and partitioned between EtOAc (200 mL) and H₂O (30 mL). The organic layer was washed with brine (20 mL), dried over Na₂SO₄, filtered and concentrated in vacuum. The residue dissolved in DCM, loaded to silica gel column (SanTai, 80 g) and purified by flash (Biotage) column chromatography (6% MeOH in DCM; flow rate: 50 mL/min) to give isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (227 mg, 0.337 mmol, 39.5% yield) as a white solid. LCMS (M+H)=675.2; Retention time (0.05% TFA)=1.60 min.

### Step 5: (2R,3S,5R)-5-(6-Amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl nonanoate

To a solution of nonanoic acid (42.2 mg, 0.267 mmol) in DCM (6 mL) was added N,N-dimethylpyridin-4-amine (27.2 mg, 0.222 mmol) and 3-(((ethylimino)methylene)amino)-N,N-dimethylpropan-1-amine hydrochloride (85 mg, 0.445 mmol) and the resulting mixture was stirred for 0.5 h at ice-water bath temperature. Then, isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.222 mmol) was added and the resulting mixture was stirred at 25° C. for 16 h. The LCMS showed the reaction was completed. The reaction mixture was concentrated, the residue was dissolved in MeOH (5 mL) and purified by reverse phase chromatography (SepaFlash@ C18 column, Biotage; 0%-100% CH3CN/10 mM aq. NH4HCO3; flow rate: 40 mL/min; column: Boston; 40 g) to give (2R,3S,5R)-5-(6-amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl nonanoate (116 mg, 0.136 mmol, 61.3% yield) as a white solid. LCMS (M+H)=815.3; Retention time (0.05% TFA)=2.02 min. ¹H NMR (400 MHz, CDCl₃) δ 7.99 (s, 1H), 7.30-7.24 (m, 2H), 7.18-7.15 (m, 2H), 7.14-7.10 (m, 1H), 6.68 (dd, J=8.0, 2.0 Hz, 2H), 6.63 (tt, J=9.2, 2.4 Hz, 1H), 6.40 (t, J=6.4 Hz, 1H), 5.98 (brs, 2H), 5.63 (dd, J=7.2, 4.8 Hz, 1H), 4.96 (quint, J=6.4 Hz, 1H), 4.35 (dd, J=11.2, 6.4 Hz, 1H), 4.28-4.22 (m, 2H), 3.81 (t, J=10.0 Hz, 1H), 2.98 (d, J=6.0 Hz, 2H), 2.78 (quint, J=7.2 Hz, 1H), 2.67-2.61 (m, 2H), 2.40 (t, J=7.6 Hz, 2H), 1.67 (quint, J=7.6 Hz, 2H), 1.35-1.25 (m, 10H), 1.16 (dd, J=6.0, 4.0 Hz, 6H), 0.88 (t, J=7.2 Hz, 3H).

### Example 109

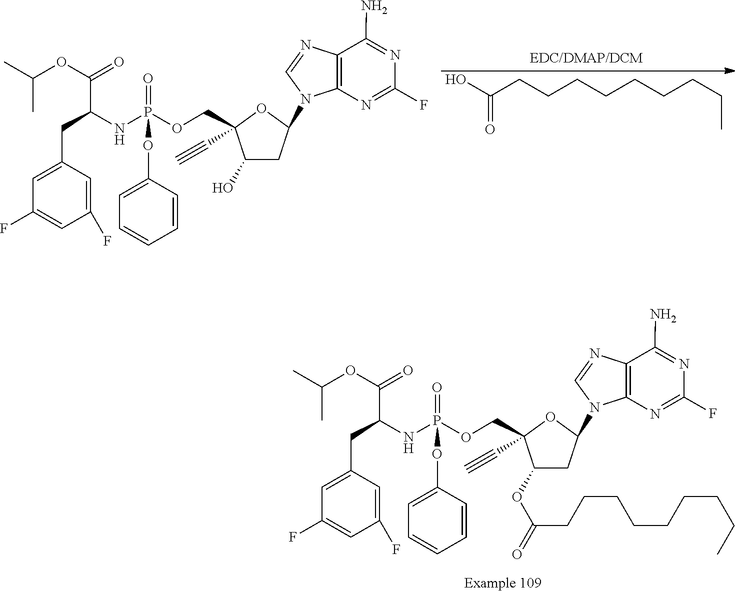

### (2R,3S,5R)-5-(6-Amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl decanoate

To a solution of decanoic acid (46.0 mg, 0.267 mmol) in DCM (6 mL) was added N,N-dimethylpyridin-4-amine (27.2 mg, 0.222 mmol) and 3-(((ethylimino)methylene)amino)-N,N-dimethylpropan-1-amine hydrochloride (85 mg, 0.445 mmol) and the resulting mixture was stirred for 0.5 h at ice-water bath temperature. Then, isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.222 mmol) was added and the resulting mixture was stirred at 25° C. for 16 h. The LCMS showed the reaction was completed. The reaction mixture was concentrated and the residue was dissolved in MeOH (5 mL) and purified by reverse phase chromatography (SepaFlash@ C18 column, Biotage; 0%-100% CH3CN/10 mM aq. NH4HCO3; flow rate: 40 mL/min; column: Boston; 40 g) to give (2R,3S,5R)-5-(6-amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl decanoate (125 mg, 0.145 mmol, 65.2% yield) as a white solid. LCMS (M+H)=829.2; Retention time (0.05% TFA)=2.08 min. ¹H NMR (400 MHz, CDCl₃) δ 8.00 (s, 1H), 7.29-7.24 (m, 2H), 7.18-7.14 (m, 2H), 7.14-7.10 (m, 1H), 6.68 (dd, J=8.0, 2.0 Hz, 2H), 6.63 (tt, J=9.2, 2.4 Hz, 1H), 6.40 (t, J=6.4 Hz, 1H), 6.07 (brs, 2H), 5.63 (dd, J=7.2, 4.4 Hz, 1H), 4.96 (quint, J=6.4 Hz, 1H), 4.35 (dd, J=10.8, 6.4 Hz, 1H), 4.27-4.22 (m, 2H), 3.87 (t, J=10.4 Hz, 1H), 2.98 (d, J=6.0 Hz, 2H), 2.78 (quint, J=7.2 Hz, 1H), 2.67-2.61 (m, 2H), 2.40 (t, J=7.6 Hz, 2H), 1.67 (quint, J=7.2 Hz, 2H), 1.35-1.25 (m, 12H), 1.16 (dd, J=6.0, 4.4 Hz, 6H), 0.88 (t, J=6.8 Hz, 3H).

### Example 110

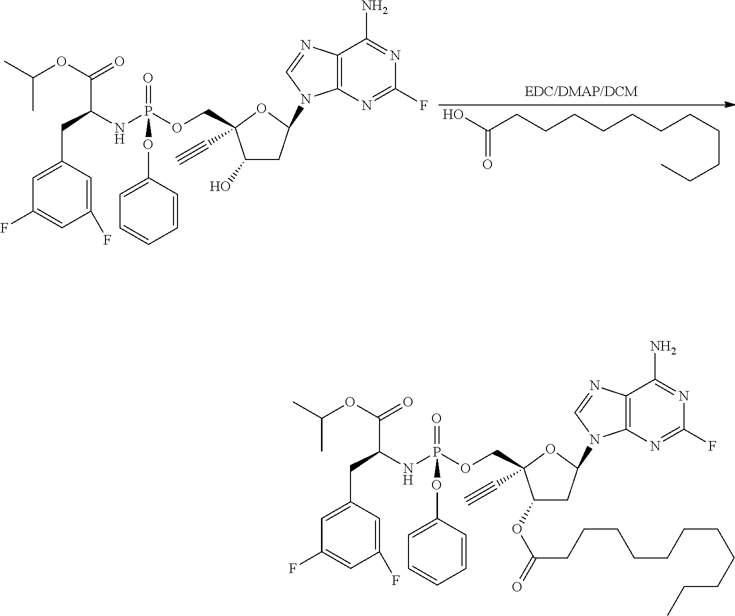

### (2R,3S,5R)-5-(6-Amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl dodecanoate

To a solution of dodecanoic acid (53.5 mg, 0.267 mmol) in DCM (6 mL) was added N,N-dimethylpyridin-4-amine (27.2 mg, 0.222 mmol) and 3-(((ethylimino)methylene)amino)-N,N-dimethylpropan-1-amine hydrochloride (85 mg, 0.445 mmol) and the resulting mixture was stirred for 0.5 h at ice-water bath temperature. Then, isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.222 mmol) was added and the resulting mixture was stirred at 25° C. for 16 h. The LCMS showed the reaction was completed. The reaction mixture was concentrated. The residue was dissolved in MeOH (5 mL) and purified by reverse phase chromatography (SepaFlash@ C18 column, Biotage; 0%-100% CH3CN/10 mM aq. NH4HCO3; flow rate: 40 mL/min; column: Boston; 40 g) to give (2R,3S,5R)-5-(6-amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl dodecanoate (141 mg, 0.161 mmol, 72.3% yield) as a white solid. LCMS (M+H)=857.3; Retention time (0.05% TFA)=2.22 min. 1H NMR (400 MHz, CDCl₃) δ 7.99 (s, 1H), 7.29-7.24 (m, 2H), 7.18-7.10 (m, 3H), 6.68 (dd, J=8.0, 2.0 Hz, 2H), 6.63 (tt, J=8.8, 2.4 Hz, 1H), 6.40 (t, J=6.4 Hz, 1H), 5.92 (brs, 2H), 5.63 (dd, J=6.8, 4.4 Hz, 1H), 4.96 (quint, J=6.4 Hz, 1H), 4.35 (dd, J=11.2, 6.4 Hz, 1H), 4.28-4.21 (m, 2H), 3.77 (t, J=10.4 Hz, 1H), 2.98 (d, J=6.0 Hz, 2H), 2.78 (quint, J=6.8 Hz, 1H), 2.67-2.61 (m, 2H), 2.40 (t, J=7.6 Hz, 2H), 1.70-1.61 (m, 2H), 1.35-1.25 (m, 16H), 1.16 (dd, J=6.0, 3.6 Hz, 6H), 0.88 (t, J=6.8 Hz, 3H).

### Example 111

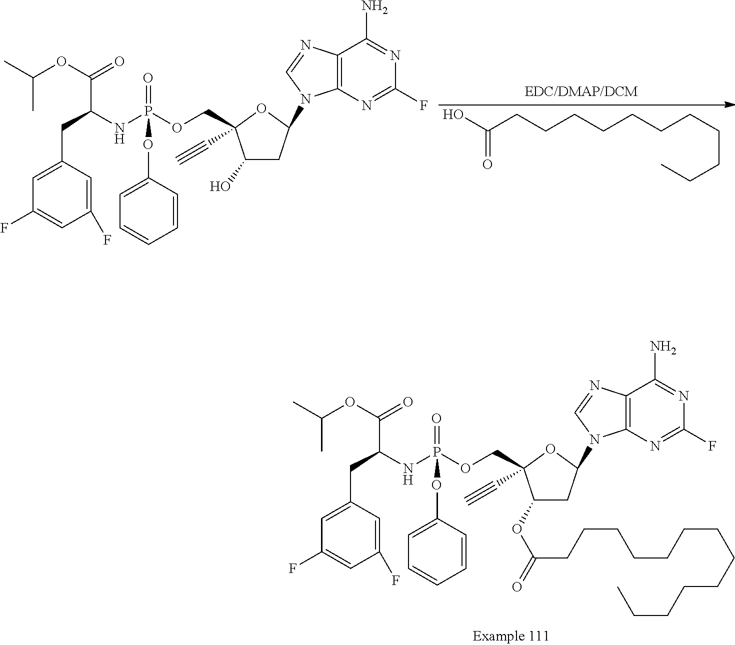

### (2R,3S,5R)-5-(6-Amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl tetradecanoate

To a solution of tetradecanoic acid (60.9 mg, 0.267 mmol) in DCM (6 mL) was added N,N-dimethylpyridin-4-amine (27.2 mg, 0.222 mmol) and 3-(((ethylimino)methylene)amino)-N,N-dimethylpropan-1-amine hydrochloride (85 mg, 0.445 mmol) and the resulting mixture was stirred for 0.5 h at ice-water bath temperature. Then, isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.222 mmol) was added and the resulting mixture was stirred at 25° C. for 16 h. The LCMS showed the reaction was completed. The reaction mixture was concentrated. The residue was dissolved in MeOH (5 mL) and purified by reverse phase chromatography (SepaFlash@ C18 column, Biotage; 0%-100% CH3CN/10 mM aq. NH4HCO3; flow rate: 40 mL/min; column: Boston; 40 g) to give (2R,3S,5R)-5-(6-amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl tetradecanoate (125 mg, 0.137 mmol, 61.8% yield) as a white solid. LCMS (M+H)=885.3; Retention time (0.05% TFA)=2.41 min. ¹H NMR (400 MHz, CDCl₃) δ 8.00 (s, 1H), 7.29-7.23 (m, 2H), 7.18-7.14 (m, 2H), 7.11 (t, J=7.6 Hz, 1H), 6.68 (dd, J=8.0, 2.0 Hz, 2H), 6.62 (tt, J=8.8, 2.4 Hz, 1H), 6.40 (t, J=6.4 Hz, 1H), 6.27 (brs, 2H), 5.63 (dd, J=7.2, 4.4 Hz, 1H), 4.96 (quint, J=6.4 Hz, 1H), 4.35 (dd, J=10.8, 6.4 Hz, 1H), 4.29-4.21 (m, 2H), 4.01 (t, J=10.4 Hz, 1H), 2.97 (d, J=6.0 Hz, 2H), 2.77 (quint, J=6.8 Hz, 1H), 2.67-2.60 (m, 2H), 2.40 (t, J=7.6 Hz, 2H), 1.66 (quint, J=7.2 Hz, 2H), 1.35-1.25 (m, 20H), 1.16 (t, J=6.0 Hz, 6H), 0.88 (t, J=6.8 Hz, 3H).

### Example 112

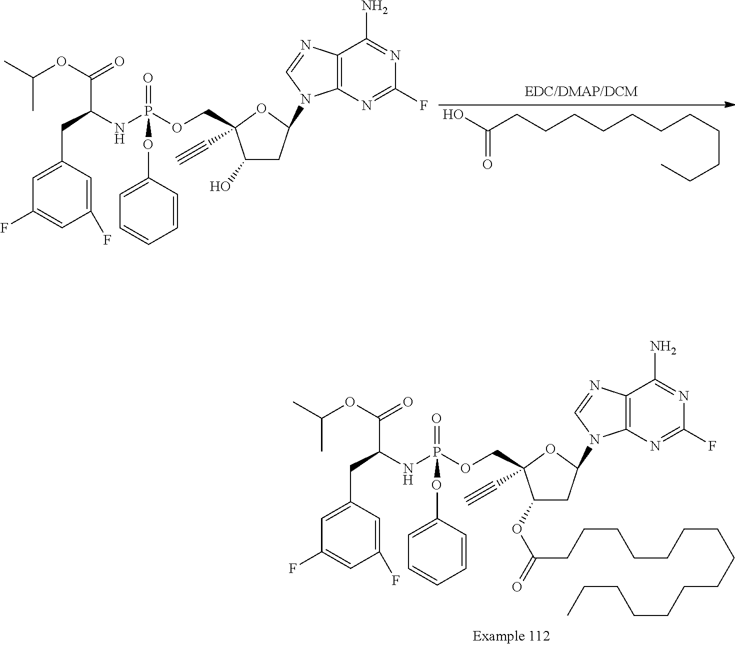

### (2R,3S,5R)-5-(6-Amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl palmitate

To a solution of palmitic acid (57.0 mg, 0.222 mmol) in DCM (6 mL) was added N,N-dimethylpyridin-4-amine (27.2 mg, 0.222 mmol) and 3-(((ethylimino)methylene)amino)-N,N-dimethylpropan-1-amine hydrochloride (85 mg, 0.445 mmol) and the resulting mixture was stirred for 0.5 h at ice-water bath temperature. Then, isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.222 mmol) was added and the resulting mixture was stirred at 25° C. for 16 h. The LCMS showed the reaction was completed. The reaction mixture was concentrated. The residue was dissolved in MeOH (5 mL) and purified by reverse phase chromatography (SepaFlash@ C18 column, Biotage; 0%-100% CH3CN/10 mM aq. NH4HCO3; flow rate: 40 mL/min; column: Boston; 40 g) to give (2R,3S,5R)-5-(6-amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl palmitate (139 mg, 0.152 mmol, 68.3% yield) as a white solid. LCMS (M+H)=914.4; Retention time (0.05% TFA)=2.64 min. ¹H NMR (400 MHz, CDCl₃) δ 8.18 (s, 1H), 7.28-7.24 (m, 2H), 7.17-7.14 (m, 2H), 7.11 (t, J=7.6 Hz, 1H), 6.68 (dd, J=7.6, 2.0 Hz, 2H), 6.63 (tt, J=8.8, 2.4 Hz, 1H), 6.40 (t, J=6.8 Hz, 1H), 6.24 (brs, 2H), 5.61 (dd, J=6.8, 4.4 Hz, 1H), 4.96 (quint, J=6.4 Hz, 1H), 4.34 (dd, J=11.2, 6.8 Hz, 1H), 4.29-4.20 (m, 2H), 3.89 (t, J=10.4 Hz, 1H), 2.98 (d, J=6.0 Hz, 2H), 2.72 (quint, J=6.8 Hz, 1H), 2.69-2.62 (m, 2H), 2.40 (t, J=7.6 Hz, 2H), 1.66 (quint, J=7.2 Hz, 2H), 1.38-1.22 (m, 24H), 1.17 (dd, J=6.4, 4.8 Hz, 6H), 0.88 (t, J=6.8 Hz, 3H).

### Example 113

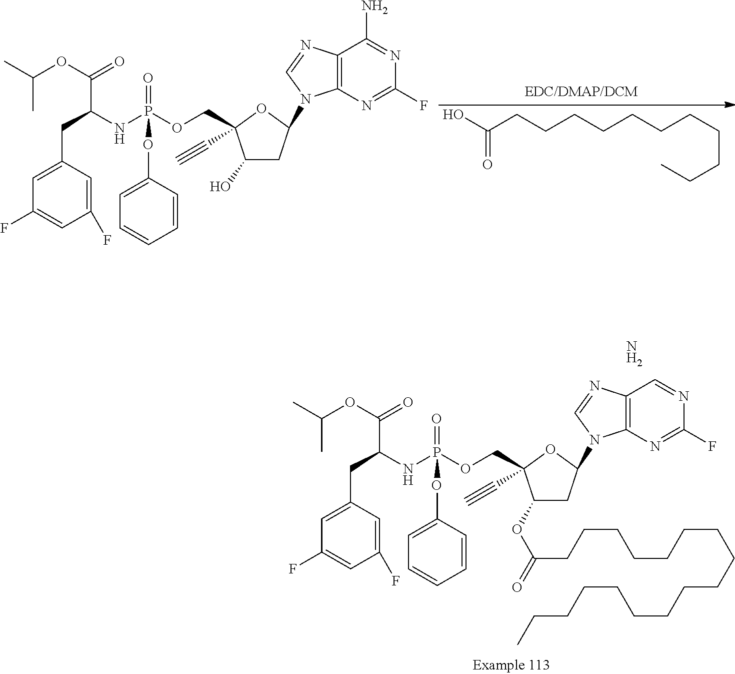

### (2R,3S,5R)-5-(6-amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl stearate

To a solution of stearic acid (127 mg, 0.445 mmol) in DCM (10 mL) was added N,N-dimethylpyridin-4-amine (54.3 mg, 0.445 mmol) and 3-(((ethylimino)methylene)amino)-N,N-dimethylpropan-1-amine hydrochloride (171 mg, 0.889 mmol) and the resulting mixture was stirred for 0.5 h at ice-water bath temperature. Then, isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (300 mg, 0.445 mmol) was added and the resulting mixture was stirred at 25° C. for 16 h. The LCMS showed the reaction was completed. The reaction mixture was concentrated. The residue was dissolved in MeOH (5 mL) and purified by reverse phase chromatography (SepaFlash@ C18 column, Biotage; 0%-100% CH3CN/10 mM aq. NH4HCO3; flow rate: 40 mL/min; column: Boston; 40 g) to give (2R,3S,5R)-5-(6-amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl stearate (127 mg, 0.135 mmol, 30.3% yield) as a white solid. LCMS (M+H)=941.3; Retention time (0.05% TFA)=3.55 min. ¹H NMR (400 MHz, CDCl₃) δ 8.03 (s, 1H), 7.31-7.26 (m, 2H), 7.18 (dd, J=8.0, 1.2 Hz, 2H), 7.14 (t, J=7.6 Hz, 1H), 6.70 (dd, J=8.0, 2.0 Hz, 2H), 6.65 (tt, J=8.8, 2.4 Hz, 1H), 6.42 (t, J=6.8 Hz, 1H), 6.14 (brs, 2H), 5.65 (dd, J=6.8, 4.4 Hz, 1H), 4.98 (quint, J=6.4 Hz, 1H), 4.37 (dd, J=11.2, 6.4 Hz, 1H), 4.30-4.24 (m, 2H), 3.91 (t, J=10.4 Hz, 1H), 2.99 (d, J=6.0 Hz, 2H), 2.79 (quint, J=6.8 Hz, 1H), 2.70-2.62 (m, 2H), 2.42 (t, J=7.6 Hz, 2H), 1.68 (quint, J=7.6 Hz, 2H), 1.39-1.25 (m, 28H), 1.18 (dd, J=6.4, 4.8 Hz, 6H), 0.90 (t, J=6.8 Hz, 3H).

### Example 114

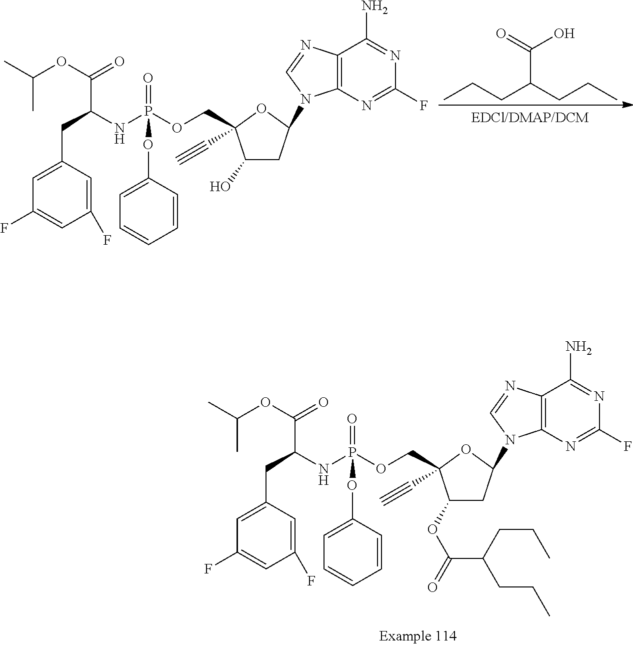

### (2R,3S,5R)-5-(6-Amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl 2-propylpentanoate

To a solution of 2-propylpentanoic acid (32.1 mg, 0.222 mmol) in DCM (6 mL) was added N,N-dimethylpyridin-4-amine (27.2 mg, 0.222 mmol) and 3-(((ethylimino)methylene)amino)-N,N-dimethylpropan-1-amine hydrochloride (85 mg, 0.445 mmol) and the resulting mixture was stirred for 0.5 h at ice-water bath temperature. Then, isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.222 mmol) was added and the resulting mixture was stirred at 25° C. for 16 h. The LCMS showed the reaction was completed. The reaction mixture was concentrated. The residue was dissolved in MeOH (5 mL) and purified by reverse phase chromatography (SepaFlash@ C18 column, Biotage; 0%-100% CH3CN/10 mM aq. NH4HCO3; flow rate: 40 mL/min; column: Boston; 40 g) to give (2R,3S,5R)-5-(6-amino-2-fluoro-9H-purin-9-yl)-2-((((S)-(((S)-3-(3,5-difluorophenyl)-1-isopropoxy-1-oxopropan-2-yl)amino)(phenoxy)phosphoryl)oxy)methyl)-2-ethynyltetrahydrofuran-3-yl 2-propylpentanoate (100 mg, 0.125 mmol, 56.2% yield) as a white solid. LCMS (M+H)=801.3; Retention time (0.05% TFA)=1.95 min. ¹H NMR (400 MHz, CDCl₃) δ 7.99 (s, 1H), 7.29-7.24 (m, 2H), 7.17-7.10 (m, 3H), 6.68 (dd, J=8.0, 2.0 Hz, 2H), 6.67-6.60 (m, 1H), 6.39 (t, J=6.4 Hz, 1H), 5.87 (brs, 2H), 5.62 (dd, J=7.2, 5.6 Hz, 1H), 4.95 (quint, J=6.4 Hz, 1H), 4.36 (dd, J=11.2, 6.4 Hz, 1H), 4.29-4.22 (m, 2H), 3.77 (t, J=10.4 Hz, 1H), 2.98 (d, J=6.0 Hz, 2H), 2.85 (quint, J=6.8 Hz, 1H), 2.70-2.62 (m, 2H), 2.51-2.43 (m, 1H), 1.71-1.60 (m, 2H), 1.53-1.43 (m, 2H), 1.37-1.30 (m, 4H), 1.16 (dd, J=6.4, 4.8 Hz, 6H), 0.91 (dt, J=7.2, 1.2 Hz, 6H).

### Example 115

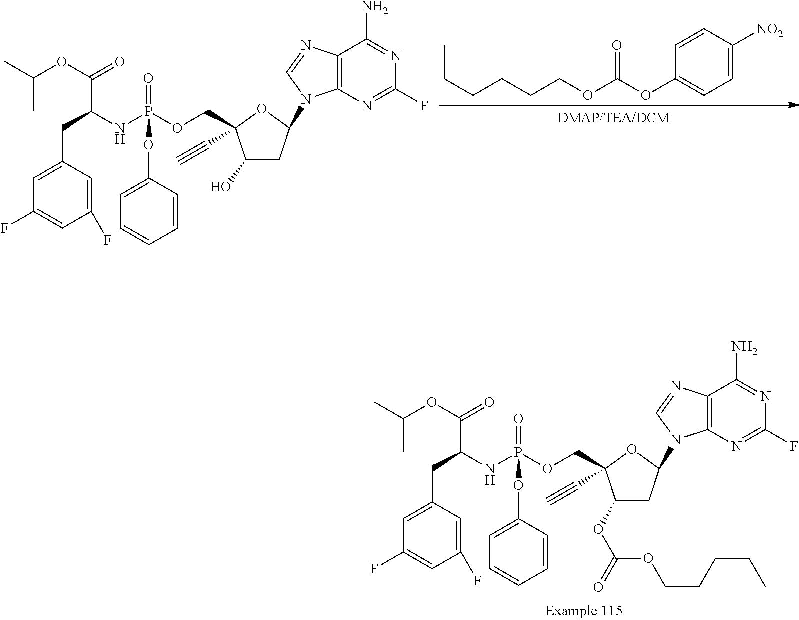

### Isopropyl (S)-2-(((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)amino)-3-(3,5-difluorophenyl)propanoate

A mixture of isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.222 mmol) in DCM (10 mL) was treated with hexyl (4-nitrophenyl) carbonate (238 mg, 0.889 mmol), N,N-dimethylpyridin-4-amine (27.2 mg, 0.222 mmol) followed by triethylamine (45.0 mg, 0.445 mmol) and the mixture was stirred at RT for 2 days. TLC showed the reaction was completed. The reaction mixture was concentrated in vacuum to give the residue. The oily residue was dissolved in MeOH (3 mL) and purified by reverse phase chromatography (Biotage; 0%-100% CH3CN/10 mM aq. NH4HCO3; flow rate: 50 mL/min; column: Boston C18, 40 g) to afford isopropyl (S)-2-(((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)amino)-3-(3,5-difluorophenyl)propanoate (105 mg, 0.131 mmol, 58.8% yield) as white solid. LCMS (M+H)=802.7; Retention time (0.1% TFA)=1.909 min. ¹H NMR (400 MHz, CDCl₃) δ 7.94 (s, 1H), 7.30-7.27 (m, 1H), 7.25 (s, 1H), 7.18-7.09 (m, 3H), 6.75-6.55 (m, 3H), 6.38 (t, J=6.6 Hz, 1H), 5.84 (s, 2H), 5.54 (dd, J=7.2, 4.8 Hz, 1H), 5.06-4.82 (m, 1H), 4.40 (dd, J=11.0, 6.3 Hz, 1H), 4.33-4.24 (m, 2H), 4.22-4.16 (m, 2H), 3.72 (t, J=10.2 Hz, 1H), 3.04-2.87 (m, 3H), 2.83-2.65 (m, 2H), 1.76-1.66 (m, 2H), 1.42-1.26 (m, 6H), 1.21-1.11 (m, 6H), 0.90 (t, J=6.9 Hz, 3H).

### Example 116

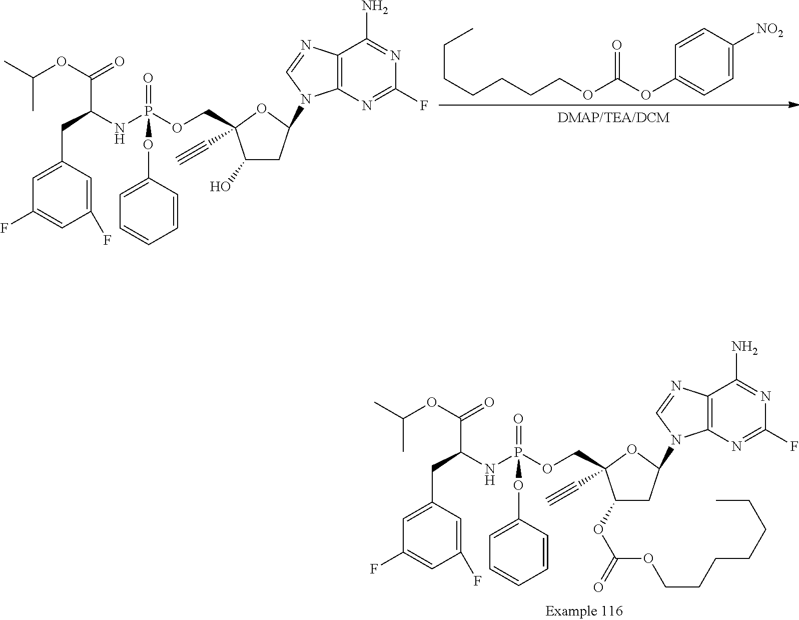

### Isopropyl (S)-2-(((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)amino)-3-(3,5-difluorophenyl)propanoate

A mixture of isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.222 mmol) in DCM (10 mL) was treated with triethylamine (0.062 mL, 0.445 mmol), DMAP (27.2 mg, 0.222 mmol) followed by heptyl (4-nitrophenyl) carbonate (250 mg, 0.889 mmol) and the mixture was stirred at RT for 2 days. TLC showed the reaction was completed. The reaction mixture was concentrated in vacuum to give the residue. The oily residue was dissolved in MeOH (3 mL) and purified by reverse phase chromatography (Biotage; 70%-100% CH3CN/10 mM aq. NH₄HCO₃); flow rate: 50 mL/min; column: Boston C18, 40 g) to afford isopropyl (S)-2-(((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)amino)-3-(3,5-difluorophenyl)propanoate (100 mg, 0.119 mmol, 53.5% yield) as white solid. LCMS (M+H)=816.7; Retention time (0.1% TFA)=1.956 min. ¹H NMR (400 MHz, CDCl₃) δ 7.95 (d, J=6.3 Hz, 1H), 7.28 (d, J=4.3 Hz, 1H), 7.24 (d, J=2.3 Hz, 1H), 7.18-7.09 (m, 3H), 6.71-6.60 (m, 3H), 6.38 (t, J=6.6 Hz, 1H), 5.93 (s, 2H), 5.54 (dt, J=16.4, 8.2 Hz, 1H), 5.14-4.86 (m, 1H), 4.40 (dd, J=11.0, 6.4 Hz, 1H), 4.34-4.12 (m, 4H), 3.79 (t, J=10.3 Hz, 1H), 3.05-2.87 (m, 3H), 2.79-2.63 (m, 2H), 1.70 (dd, J=12.3, 4.2 Hz, 2H), 1.45-1.22 (m, 8H), 1.19-1.07 (m, 6H), 0.89 (t, J=6.8 Hz, 3H).

### Example 117

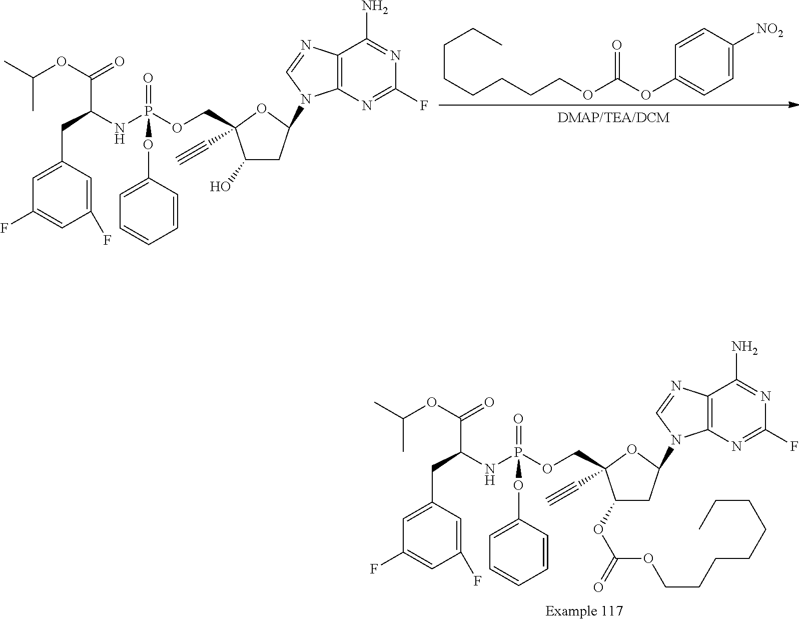

### Isopropyl (S)-2-(((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)amino)-3-(3,5-difluorophenyl)propanoate

A mixture of isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.222 mmol) in DCM (10 mL) was treated with 4-nitrophenyl octyl carbonate (263 mg, 0.889 mmol), N,N-dimethylpyridin-4-amine (27.2 mg, 0.222 mmol) followed by triethylamine (45.0 mg, 0.445 mmol) and the mixture was stirred at RT for 2 days. TLC showed the reaction was completed. The reaction mixture was concentrated in vacuum to give the residue. The oily residue was dissolved in MeOH (3 mL) and purified by reverse phase chromatography (Biotage, gradient: 70%-100% CH3CN/10 mM aq. NH₄HCO₃; flow rate: 50 mL/min; column: Boston C18, 40 g) to afford isopropyl (S)-2-(((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)amino)-3-(3,5-difluorophenyl)propanoate (100 mg, 0.117 mmol, 52.6% yield) as white solid. LCMS (M+H)=830.7; Retention time (0.1% TFA)=2.003 min. ¹H NMR (400 MHz, CDCl₃) δ 7.95 (d, J=6.3 Hz, 1H), 7.28 (d, J=4.3 Hz, 1H), 7.24 (d, J=2.3 Hz, 1H), 7.18-7.09 (m, 3H), 6.71-6.60 (m, 3H), 6.38 (t, J=6.6 Hz, 1H), 5.93 (s, 2H), 5.54 (dt, J=16.4, 8.2 Hz, 1H), 5.14-4.86 (m, 1H), 4.40 (dd, J=11.0, 6.4 Hz, 1H), 4.34-4.12 (m, 4H), 3.79 (t, J=10.3 Hz, 1H), 3.05-2.87 (m, 3H), 2.79-2.63 (m, 2H), 1.70 (dd, J=12.3, 4.2 Hz, 2H), 1.45-1.22 (m, 10H), 1.19-1.07 (m, 6H), 0.89 (t, J=6.8 Hz, 3H).

### Example 118

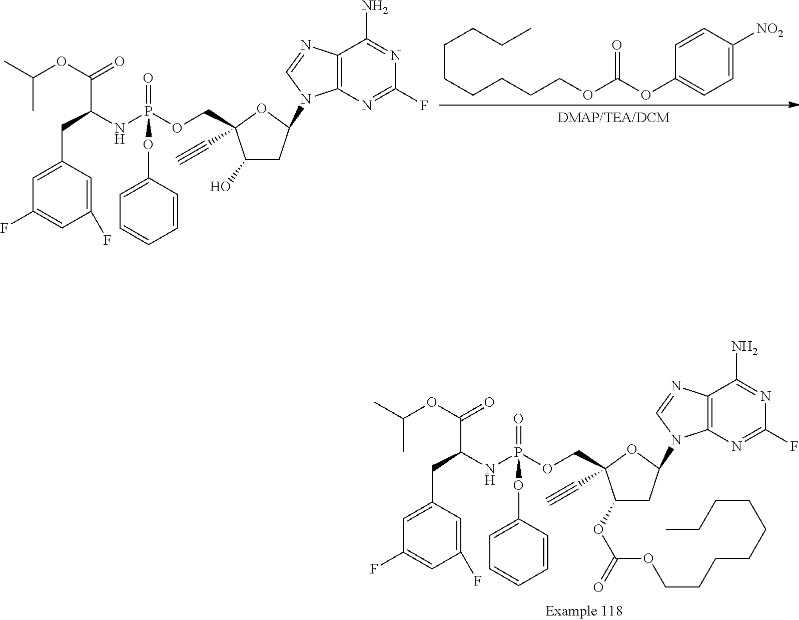

### Isopropyl (S)-2-(((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)amino)-3-(3,5-difluorophenyl)propanoate

A mixture of isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (200 mg, 0.296 mmol) was treated with 4-nitrophenyl nonyl carbonate (367 mg, 1.186 mmol), N,N-dimethylpyridin-4-amine (36.2 mg, 0.296 mmol) followed by triethylamine (60.0 mg, 0.593 mmol) and the mixture was stirred at RT for 2 days. TLC showed the reaction was completed. The reaction mixture was concentrated in vacuum to give the residue which was dissolved in DCM (3 mL), loaded to silica column (SanTai, 40 g) and purified by flash (Biotage) chromatography (0-5% MeOH/DCM, 50 mL/min) to afford isopropyl (S)-2-(((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)amino)-3-(3,5-difluorophenyl)propanoate (105 mg, 0.124 mmol, 41.9% yield) as white solid. LCMS (M+H)=844.7; Retention time (0.1% TFA)=2.635 min. ¹H NMR (400 MHz, CDCl₃) δ 7.95 (s, 1H), 7.25 (d, J=7.8 Hz, 2H), 7.18-7.07 (m, 3H), 6.77-6.56 (m, 3H), 6.38 (t, J=6.6 Hz, 1H), 6.34-5.96 (m, 2H), 5.54 (dd, J=7.1, 4.7 Hz, 1H), 5.00-4.89 (m, 1H), 4.40 (dd, J=11.0, 6.5 Hz, 1H), 4.34-4.13 (m, 4H), 3.94 (s, 1H), 2.94 (dd, J=35.0, 6.6 Hz, 3H), 2.76-2.66 (m, 2H), 1.75-1.63 (m, 2H), 1.44-1.21 (m, 12H), 1.18-1.08 (m, 6H), 0.88 (t, J=6.8 Hz, 3H).

### Example 119

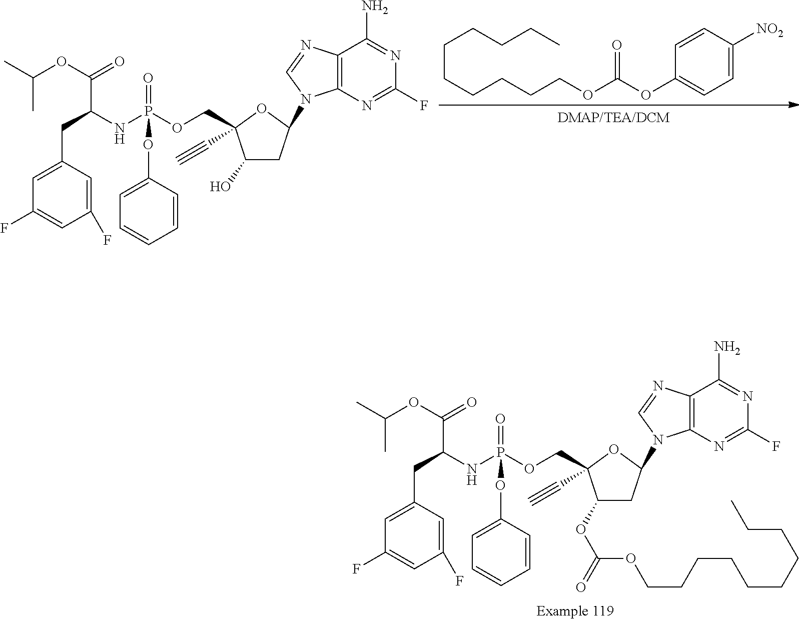

### Isopropyl (S)-2-(((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)amino)-3-(3,5-difluorophenyl)propanoate

A mixture of isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (200 mg, 0.296 mmol) in DCM (10 mL) was treated with decyl (4-nitrophenyl) carbonate (384 mg, 1.186 mmol), N,N-dimethylpyridin-4-amine (36.2 mg, 0.296 mmol) followed by triethylamine (60.0 mg, 0.593 mmol) and the mixture was stirred at RT for 2 days. TLC showed the reaction was completed. The reaction mixture was concentrated in vacuum to give the residue. The crude product was dissolved in DCM (3 mL), loaded to silica column (SanTai, 40 g), and purified by flash (Biotage) chromatography (0-5% MeOH/DCM, 50 mL/min) to afford isopropyl (S)-2-(((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)amino)-3-(3,5-difluorophenyl)propanoate (110 mg, 0.128 mmol, 43.2% yield) as white solid. LCMS (M+H)=858.7; Retention time (0.1% TFA)=2.715 min. ¹H NMR (400 MHz, CDCl₃) δ 7.95 (s, 1H), 7.25 (d, J=7.6 Hz, 2H), 7.18-7.07 (m, 3H), 6.77-6.54 (m, 3H), 6.37 (t, J=6.6 Hz, 1H), 6.35-5.95 (m, 2H), 5.54 (dd, J=7.1, 4.7 Hz, 1H), 5.01-4.88 (m, 1H), 4.40 (dd, J=11.0, 6.5 Hz, 1H), 4.36-4.13 (m, 4H), 3.96 (s, 1H), 3.03-2.84 (m, 3H), 2.80-2.64 (m, 2H), 1.74-1.63 (m, 2H), 1.39-1.20 (m, 14H), 1.16 (dd, J=6.2, 4.8 Hz, 6H), 0.88 (t, J=6.8 Hz, 3H).

### Example 120

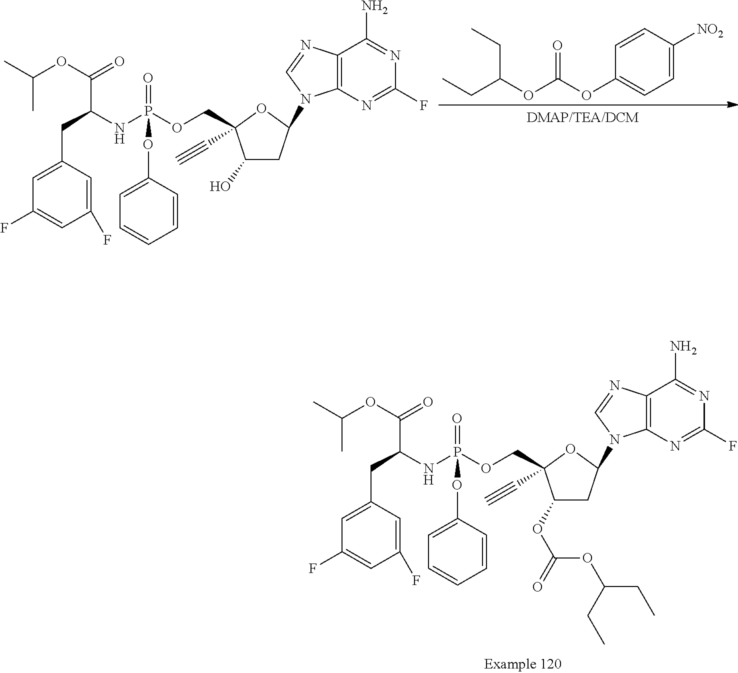

### Isopropyl (S)-2-(((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)amino)-3-(3,5-difluorophenyl)propanoate

### (GSK4422353A)

A mixture of isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (200 mg, 0.296 mmol) in DCM (10 mL) was treated with triethylamine (60.0 mg, 0.593 mmol), N,N-dimethylpyridin-4-amine (36.2 mg, 0.296 mmol) followed by 4-nitrophenyl pentan-3-yl carbonate (300 mg, 1.186 mmol), and the mixture was stirred at RT for 2 days. TLC showed the reaction was completed. The reaction mixture was concentrated in vacuum to give the residue. The crude product was dissolved in DCM (3 mL), loaded to silica column (SanTai, 40 g), and purified by flash (Biotage) chromatography (0-5% MeOH/DCM, 50 mL/min) to afford isopropyl (S)-2-(((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)amino)-3-(3,5-difluorophenyl)propanoate (90 mg, 0.114 mmol, 38.5% yield) as white solid. LCMS (M+H)=788.7; Retention time (0.1% TFA)=2.339 min. ¹H NMR (400 MHz, CDCl₃) δ 7.95 (s, 1H), 7.28 (d, J=2.4 Hz, 1H), 7.24 (s, 1H), 7.18-7.08 (m, 3H), 6.76-6.55 (m, 3H), 6.39 (t, J=6.7 Hz, 1H), 6.36-5.88 (m, 2H), 5.53 (dd, J=7.1, 4.3 Hz, 1H), 5.01-4.89 (m, 1H), 4.68-4.60 (m, 1H), 4.39 (d, J=6.5 Hz, 1H), 4.35-4.20 (m, 2H), 3.91 (s, 1H), 3.05-2.83 (m, 3H), 2.67 (s, 2H), 1.66 (td, J=7.4, 3.9 Hz, 4H), 1.20-1.09 (m, 6H), 0.95 (td, J=7.4, 1.8 Hz, 6H).

### Example 121

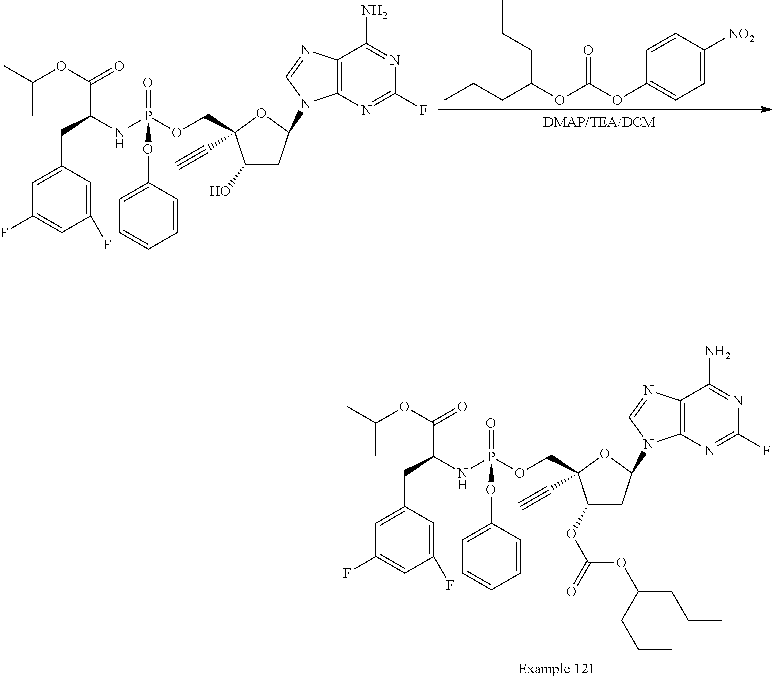

### Isopropyl (S)-2-(((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)amino)-3-(3,5-difluorophenyl)propanoate

A mixture of isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (200 mg, 0.296 mmol) in DCM (10 mL) was treated with triethylamine (0.082 mL, 0.593 mmol), N,N-dimethylpyridin-4-amine (36.2 mg, 0.296 mmol) followed by heptan-4-yl (4-nitrophenyl) carbonate (334 mg, 1.186 mmol), and the mixture was stirred at RT for 2 days. TLC showed the reaction was completed. The reaction mixture was concentrated in vacuum to give the residue. The crude product was dissolved in DCM (3 mL), loaded to silica column (SanTai, 40 g), and purified by flash (Biotage) chromatography (0-5% MeOH/DCM, 50 mL/min) to afford isopropyl (S)-2-(((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)amino)-3-(3,5-difluorophenyl)propanoate (90 mg, 0.110 mmol, 37.2% yield) as white solid. LCMS (M+H)=816.7; Retention time (0.1% TFA)=2.486 min. ¹H NMR (400 MHz, CDCl₃) δ 7.95 (s, 1H), 7.28 (s, 1H), 7.24 (s, 1H), 7.18-7.07 (m, 3H), 6.69 (d, J=6.1 Hz, 3H), 6.39 (s, 1H), 6.29-5.77 (m, 2H), 5.59-5.42 (m, 1H), 5.03-4.88 (m, 1H), 4.84-4.68 (m, 1H), 4.46-4.37 (m, 1H), 4.35-4.19 (m, 2H), 3.84 (s, 1H), 2.98 (d, J=6.1 Hz, 3H), 2.66 (s, 2H), 1.55 (d, J=5.6 Hz, 4H), 1.38 (dd, J=12.0, 5.2 Hz, 4H), 1.16 (t, J=5.7 Hz, 6H), 0.93 (dd, J=12.5, 7.2 Hz, 6H).

### Example 122

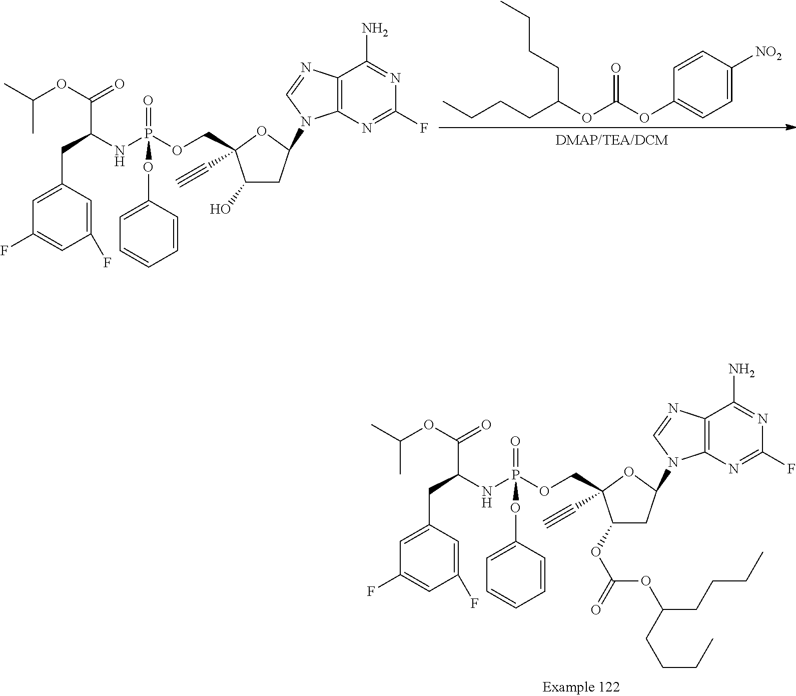

### Isopropyl (S)-2-(((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)amino)-3-(3,5-difluorophenyl)propanoate

A mixture of isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (200 mg, 0.296 mmol) in DCM (10 mL) was treated with triethylamine (0.082 mL, 0.593 mmol), N,N-dimethylpyridin-4-amine (36.2 mg, 0.296 mmol) followed by 4-nitrophenyl nonan-5-yl carbonate (367 mg, 1.186 mmol), and the mixture was stirred at RT for 2 days. TLC showed the reaction was completed. The reaction mixture was concentrated in vacuum to give the residue. The crude product was dissolved in DCM (3 mL), loaded to silica column (SanTai, 40 g), and purified by flash (Biotage) chromatography (0-5% MeOH/DCM, 50 mL/min) to afford isopropyl (S)-2-(((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)amino)-3-(3,5-difluorophenyl)propanoate (120 mg, 0.142 mmol, 47.9% yield) as white solid. LCMS (M+H)=844.7; Retention time (0.1% TFA)=2.600 min. ¹H NMR (400 MHz, CDCl₃) δ 7.96 (s, 1H), 7.28 (s, 1H), 7.24 (s, 1H), 7.18-7.08 (m, 3H), 6.67 (dd, J=13.7, 11.8 Hz, 3H), 6.39 (s, 1H), 6.23-5.86 (m, 2H), 5.52 (d, J=2.9 Hz, 1H), 5.00-4.86 (m, 1H), 4.81-4.68 (m, 1H), 4.39 (d, J=6.5 Hz, 1H), 4.33 (s, 2H), 3.93-3.78 (m, 1H), 2.98 (d, J=6.1 Hz, 3H), 2.66 (s, 2H), 1.67-1.51 (m, 4H), 1.33 (dd, J=7.1, 3.4 Hz, 8H), 1.19-1.10 (m, 6H), 0.90 (q, J=6.9 Hz, 6H).

### Example 123

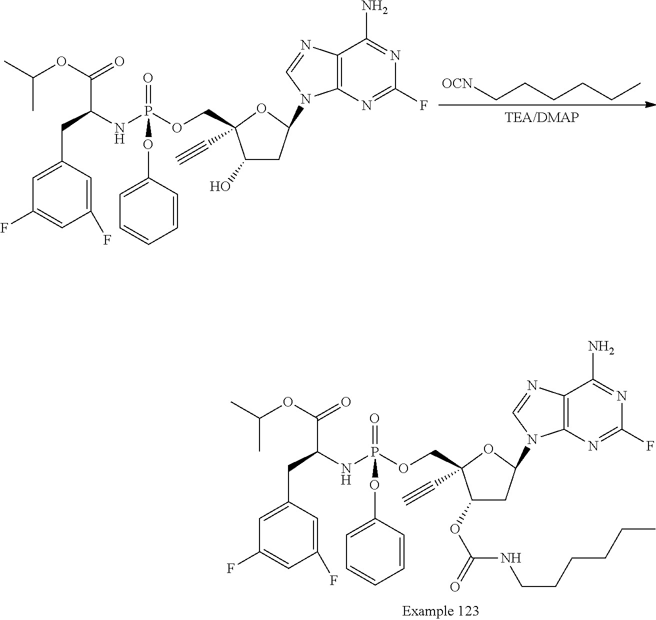

### Isopropyl (S)-2-(((S)-(((2R,3S,5R)-5-(6-amino-2-fluoro-9H-purin-9-yl)-2-ethynyl-3-((hexylcarbamoyl)oxy)tetrahydrofuran-2-yl)methoxy)(phenoxy)phosphoryl)amino)-3-(3,5-difluorophenyl)propanoate

A mixture of isopropyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy) phosphoryl)amino)-3-(3,5-difluorophenyl)propanoate (200 mg, 0.296 mmol) and DMAP (72.4 mg, 0.593 mmol) in DCM (5 mL) was treated with TEA (0.124 mL, 0.889 mmol) followed by 1-isocyanatohexane (0.6 mL, 4.12 mmol), and the mixture was stirred at 35° C. for 6 h. LCMS showed the reaction was finished. The reaction mixture was concentrated and the residue was dissolved in DCM (2 mL), loaded to silica gel column (SanTai, 25 g), and purified by flash (Biotage) column chromatography (0-3% MeOH in DCM; flow rate: 30 mL/min) to give isopropyl (S)-2-(((S)-(((2R,3S,5R)-5-(6-amino-2-fluoro-9H-purin-9-yl)-2-ethynyl-3-((hexylcarbamoyl)oxy)tetrahydrofuran-2-yl)methoxy)(phenoxy)phosphoryl)amino)-3-(3,5-difluorophenyl)propanoate (94 mg, 0.112 mmol, 37.8% yield) as a white solid. LCMS (M+H)=802.2; Retention time (0.1% TFA)=1.90 min. ¹H NMR (400 MHz, CDCl₃) δ 8.10 (s, 1H), 7.29-7.24 (m, 2H), 7.16 (d, J=8.4 Hz, 2H), 7.11 (t, J=7.2 Hz, 1H), 6.70 (dd, J=7.6, 2.0 Hz, 2H), 6.63 (tt, J=8.8, 2.0 Hz, 1H), 6.38 (t, J=6.4 Hz, 1H), 6.12 (brs, 2H), 5.54 (t, J=6.0 Hz, 1H), 4.95 (quint, J=6.4 Hz, 1H), 4.89 (t, J=6.0 Hz, 1H), 4.33 (dd, J=11.2, 6.4 Hz, 1H), 4.28-4.21 (m, 2H), 3.93 (t, J=10.4 Hz, 1H), 3.21 (q, J=6.8 Hz, 2H), 2.99 (d, J=6.0 Hz, 2H), 2.70-2.64 (m, 3H), 1.53 (quint, J=6.8 Hz, 2H), 1.35-1.27 (m, 6H), 1.16 (t, J=6.8 Hz, 6H), 0.89 (t, J=6.8 Hz, 3H).

### Example 124

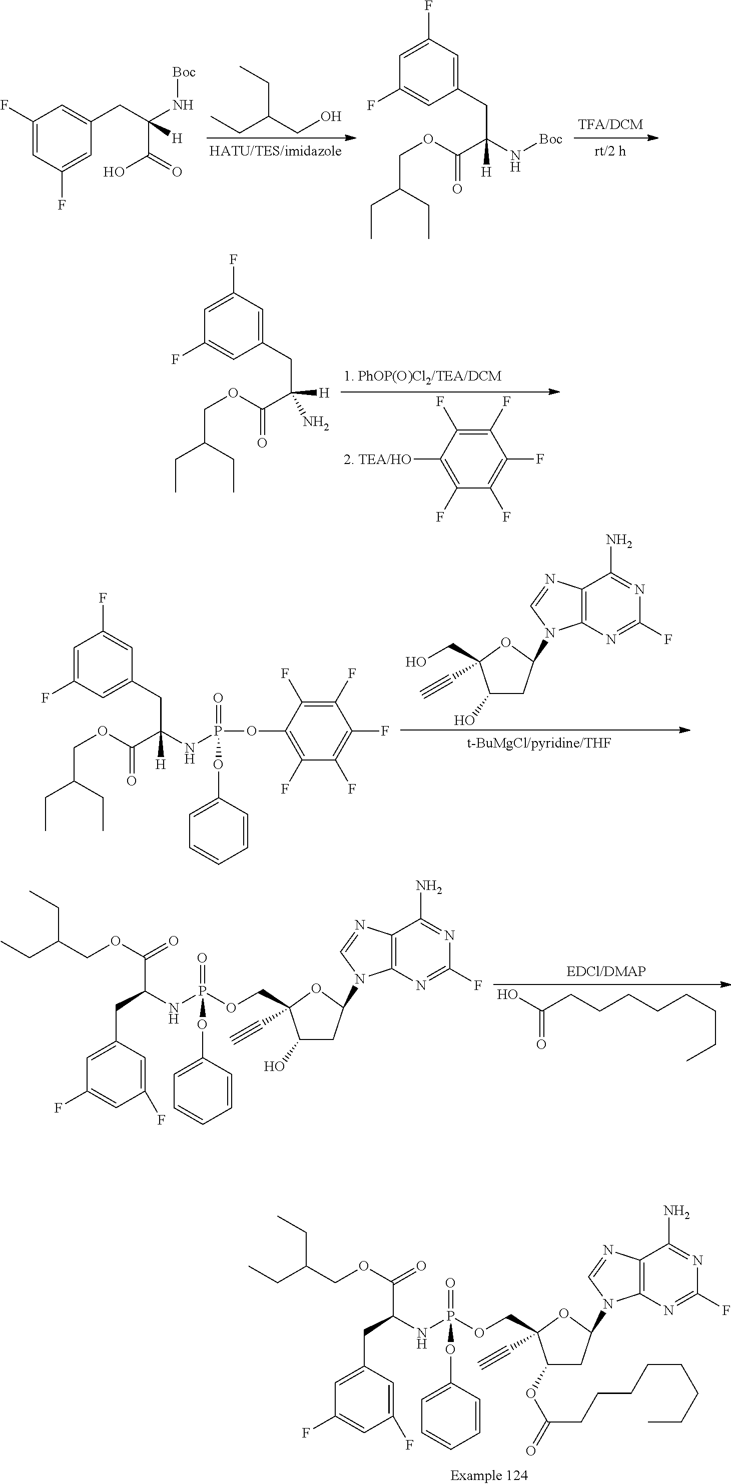

### Step 1: 2-Ethylbutyl (S)-2-((tert-butoxycarbonyl)amino)-3-(3,5-difluorophenyl)propanoate

A mixture of 2-ethylbutan-1-ol (13.57 g, 133 mmol), EDC (19.09 g, 100 mmol) in DCM (400 mL) was added DMAP (1.622 g, 13.28 mmol) at 0° C. After stirring for 30 min, (S)-2-((tert-butoxycarbonyl)amino)-3-(3,5-difluorophenyl)propanoic acid (20 g, 66.4 mmol) in DCM (20 mL) was added and stirred at 25° C. for 2 h. LCMS showed the presence of new compound. The reaction was diluted with water (250 mL), organic layer separated and aqueous layer extracted with DCM (150 mL×3). The combined organic layers were washed with brine (150 mL), dried with Na₂SO₄, filtered and concentrated in vacuum to give the residue. The crude product was dissolved in DCM (20 mL), loaded to silica column (SanTa, 330 g), and purified by flash (Biotage) chromatography (0-5% EtOAc/pet. ether, 50 mL/min) to give 2-ethylbutyl (S)-2-((tert-butoxycarbonyl)amino)-3-(3,5-difluorophenyl)propanoate (18 g, 46.7 mmol, 70.3% yield) as colourless oil. ¹H NMR (400 MHz, CDCl₃) δ 6.78-6.56 (m, 3H), 5.06 (d, J=7.6 Hz, 1H), 4.57 (dd, J=13.5, 6.2 Hz, 1H), 4.04 (ddd, J=27.1, 10.9, 5.8 Hz, 2H), 3.07 (ddd, J=34.2, 13.8, 6.0 Hz, 2H), 1.56-1.17 (m, 14H), 0.88 (t, J=7.5 Hz, 6H).

### Step 2: 2-Ethylbutyl (S)-2-amino-3-(3,5-difluorophenyl)propanoate

To a solution of 2-ethylbutyl (S)-2-((tert-butoxycarbonyl)amino)-3-(3,5-difluorophenyl)propanoate (18 g, 46.7 mmol) in DCM (200 mL) was added TFA (36.0 mL, 467 mmol) at −10° C. under N₂. The reaction was stirred for 16 h at 25° C. under N₂. TLC showed the reaction was completed. The reaction was concentrated in vacuum, the residue was taken up in water (500 mL), pH was adjusted 7 with aq. NaHCO₃ and extracted with EtOAc (250 mL×3). The combined organic layers were washed with brine (150 mL), dried over sodium sulfate and evaporated to give 2-ethylbutyl (S)-2-amino-3-(3,5-difluorophenyl)propanoate (13 g, 45.6 mmol, 98% yield) as yellow oil. ¹H NMR (400 MHz, CDCl₃) δ 6.85-6.62 (m, 3H), 4.11-3.94 (m, 2H), 3.79 (dd, J=7.3, 6.0 Hz, 1H), 3.00 (ddd, J=21.1, 13.7, 6.7 Hz, 2H), 2.77 (s, 2H), 1.48 (dt, J=12.6, 6.3 Hz, 1H), 1.39-1.17 (m, 4H), 0.87 (td, J=7.4, 0.7 Hz, 6H).

### Step 3: 2-Ethylbutyl (S)-3-(3,5-difluorophenyl)-2-(((S)-(perfluorophenoxy)(phenoxy)phosphoryl)amino)propanoate

To a solution of 2-ethylbutyl (S)-2-amino-3-(3,5-difluorophenyl)propanoate (13 g, 45.6 mmol) in anhydrous DCM (200 mL) was added dropwise triethylamine (6.97 mL, 50.1 mmol) at −70° C. over 15 min. To this mixture was added a solution of phenyl phosphorodichloridate (9.52 g, 45.1 mmol) in anhydrous DCM (35 mL) over 1 h. The reaction mixture was stirred at this temperature for additional 30 min and then allowed to warm to 0° C. over 2 h and stirred for additional 1 h. To this mixture was added a solution of 2,3,4,5,6-pentafluorophenol (8.30 g, 45.1 mmol) and triethylamine (6.97 mL, 50.1 mmol) in dichloromethane (30 mL) over 20 min. The crude mixture was allowed to stir at 0° C. for 4 h. TLC showed the reaction was completed. The white solid (triethylaminehydrochloride) was filtered off and washed with dichloromethane (50 mL). The filtrate was concentrated under reduced pressure, the residue was triturated with TBME (250 mL), and the triethylaminehydrochloride salt was removed by filtration. The cake was washed with TBME (2×50 mL), and the combined filtrate was concentrated under reduced pressure to give 26 g crude product diasteromer mixture The mixture was triturated with 10% EtOAc in hexanes (50 mL) and solids were collected by filtration to give 2-ethylbutyl (S)-3-(3,5-difluorophenyl)-2-(((S)-(perfluorophenoxy)(phenoxy)phosphoryl)amino)propanoate (5 g, 8.23 mmol, 18.07% yield) as a white solid (>98% de as determined by P³¹NMR). ¹H NMR (400 MHz, CDCl₃) δ 7.37 (t, J=7.9 Hz, 2H), 7.27-7.15 (m, 3H), 6.67 (tt, J=9.0, 2.3 Hz, 1H), 6.63-6.53 (m, 2H), 4.44 (tt, J=9.9, 5.9 Hz, 1H), 4.17-3.91 (m, 3H), 3.08-3.00 (m, 2H), 1.47 (dd, J=12.5, 6.3 Hz, 1H), 1.34-1.24 (m, 4H), 0.95-0.77 (m, 7H). ³¹P NMR (162 MHz, CDCl₃) δ−1.60 (s). LCMS (M+H)=608.0.

### Step 4: 2-Ethylbutyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a solution of (2R,3S,5R)-5-(6-amino-2-fluoro-9H-purin-9-yl)-2-ethynyl-2-(hydroxymethyl)tetrahydrofuran-3-ol (0.3 g, 1.023 mmol) in THF (50 mL) and pyridine (2.5 mL) was added dropwise tert-butylmagnesium chloride (2.046 mL, 2.046 mmol) at −15° C., and the reaction mixture was stirred at −15° C. for 1 h. Then, to the reaction mixture was added a solution of 2-ethylbutyl (S)-3-(3,5-difluorophenyl)-2-(((S)-(perfluorophenoxy)(phenoxy)phosphoryl)amino)propanoate (0.746 g, 1.228 mmol) in THF (15 mL), and the resulting reaction mixture was stirred at −15° C. for 2 h. The reaction was quenched with aq. NH₄Cl (4 mL) and partitioned between EtOAc (200 mL) and H₂O (30 mL). The organic layer was washed with brine (20 mL), dried over Na₂SO₄, filtered and concentrated in vacuum. The residue was dissolved in MeOH and purified by reverse phase chromatography (SepaFlash@ C18 column, Biotage; gradient: 0%-60% ACN in water (0.1% TFA); flow rate: 50 mL/min; column: Boston; 80 g) to give 2-ethylbutyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (300 mg, 0.416 mmol, 40.6% yield) as a white solid. LCMS (M+H)=717.2; Retention time (0.05% TFA)=1.75 min.

### Step 5: (2R,3S,5R)-5-(6-Amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl nonanoate

To a solution of nonanoic acid (43.1 mg, 0.272 mmol) in DCM (10 mL) was added N,N-dimethylpyridin-4-amine (25.6 mg, 0.209 mmol) and 3-(((ethylimino)methylene)amino)-N,N-dimethylpropan-1-amine hydrochloride (80 mg, 0.419 mmol), and the resulting mixture was stirred for 0.5 h at ice-water bath temperature. Then, 2-ethylbutyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.209 mmol) was added, and the resulting mixture was stirred at 25° C. for 4 h. LCMS showed the reaction was completed. The reaction was concentrated in vacuum to give the residue. The residue was dissolved in MeOH (3 mL) and purified by reverse flash chromatography (Biotage, gradient: 70%-100% ACN/10 mM aq. NH₄HCO₃; flow rate: 30 mL/min; column: Boston C18, 40 g) to give (2R,3S,5R)-5-(6-amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl nonanoate (100 mg, 0.117 mmol, 55.8% yield) as a white solid. LCMS: LCMS (M+H)=857.7; Retention time (10 mMNH4HCO3)=2.62 min. ¹H NMR (400 MHz, CDCl₃) δ 7.99 (s, 1H), 7.24 (s, 2H), 7.13 (dd, J=16.7, 7.9 Hz, 3H), 6.72-6.57 (m, 3H), 6.40 (t, J=6.6 Hz, 1H), 6.10 (s, 2H), 5.63 (dd, J=7.0, 4.6 Hz, 1H), 4.33 (ddd, J=9.8, 8.6, 4.9 Hz, 2H), 4.29-4.22 (m, 1H), 4.02-3.93 (m, 2H), 3.88 (t, J=10.3 Hz, 1H), 2.99 (d, J=6.2 Hz, 2H), 2.77 (dt, J=13.9, 7.1 Hz, 1H), 2.69-2.57 (m, 2H), 2.40 (t, J=7.6 Hz, 2H), 1.73-1.60 (m, 2H), 1.42 (dt, J=12.6, 6.3 Hz, 1H), 1.36-1.22 (m, 14H), 0.86 (dt, J=14.9, 6.9 Hz, 9H).

### Example 125

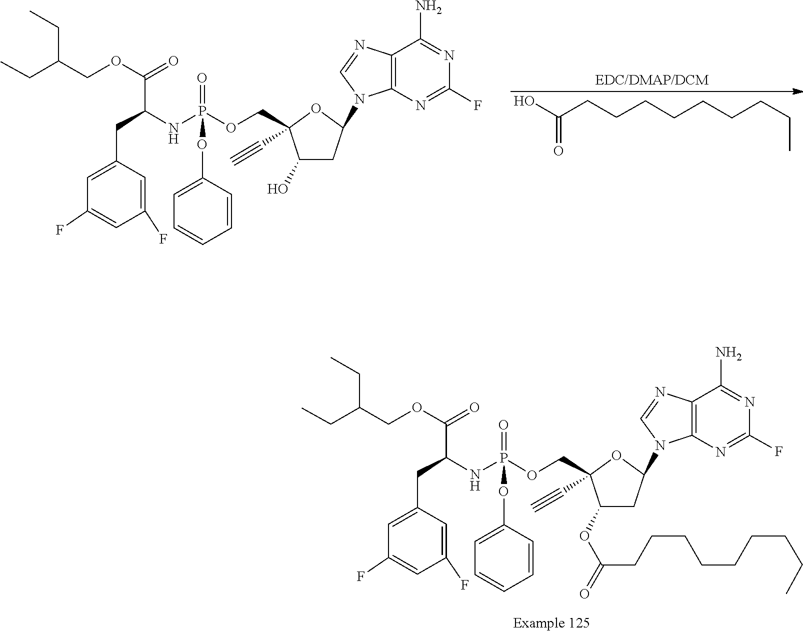 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1O ; CCCCCCCCCC(=O)O ; C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCC]

### (2R,3S,5R)-5-(6-Amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl decanoate

To a solution of decanoic acid (46.9 mg, 0.272 mmol) in DCM (10 mL) was added N,N-dimethylpyridin-4-amine (25.6 mg, 0.209 mmol) and 3-(((ethylimino)methylene)amino)-N,N-dimethylpropan-1-amine hydrochloride (80 mg, 0.419 mmol), and the resulting mixture was stirred for 0.5 h at ice-water bath temperature. Then, 2-ethylbutyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.209 mmol) was added, and the resulting mixture was stirred at 25° C. for 4 h. LCMS showed the reaction was completed. The reaction was concentrated in vacuum to give the residue. The residue was dissolved in MeOH (3 mL) and purified by reverse flash chromatography (Biotage, gradient: 70%-100% ACN/10 mM aq. NH₄HCO₃; flow rate: 30 mL/min; column: Boston C18, 40 g) to give (2R,3S,5R)-5-(6-amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl decanoate (100 mg, 0.115 mmol, 54.9% yield) as a white solid. LCMS (M+H)=870.7; Retention time (10 mMNH4HCO3)=2.699 min. ¹H NMR (400 MHz, CDCl₃) δ 7.99 (s, 1H), 7.28 (s, 1H), 7.25 (s, 1H), 7.13 (dd, J=16.3, 7.9 Hz, 3H), 6.73-6.58 (m, 3H), 6.40 (t, J=6.6 Hz, 1H), 6.05 (s, 2H), 5.63 (dd, J=7.1, 4.6 Hz, 1H), 4.33 (ddd, J=9.8, 8.6, 4.9 Hz, 2H), 4.28-4.21 (m, 1H), 4.02-3.92 (m, 2H), 3.86 (t, J=10.3 Hz, 1H), 2.99 (d, J=6.1 Hz, 2H), 2.77 (dt, J=13.9, 7.0 Hz, 1H), 2.69-2.60 (m, 2H), 2.40 (t, J=7.6 Hz, 2H), 1.67 (dt, J=15.0, 7.4 Hz, 2H), 1.42 (dt, J=12.6, 6.3 Hz, 1H), 1.37-1.20 (m, 16H), 0.86 (dt, J=14.9, 7.0 Hz, 9H).

### Example 126

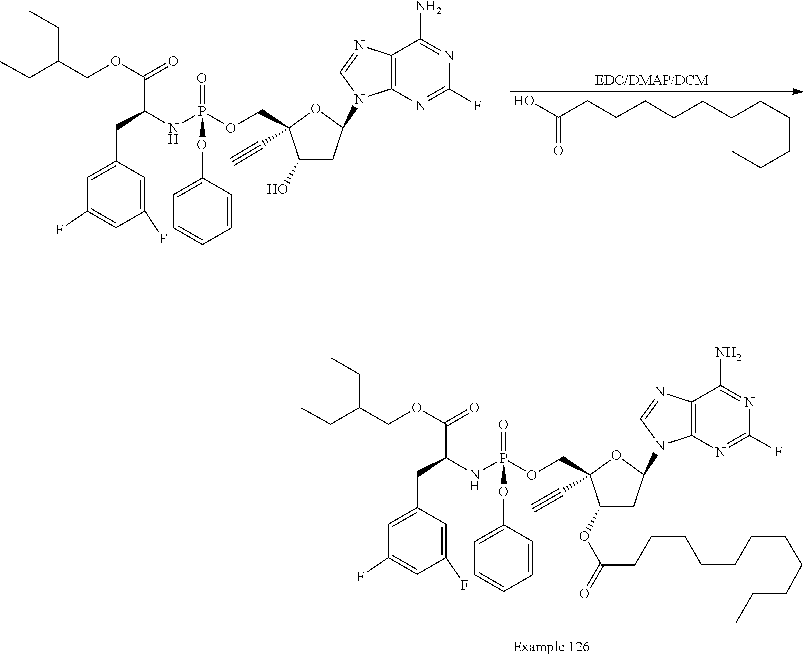 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1O ; CCCCCCCCCCCC(=O)O ; C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCC]

### (2R,3S,5R)-5-(6-Amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl dodecanoate

To a solution of dodecanoic acid (54.5 mg, 0.272 mmol) in DCM (10 mL) was added N,N-dimethylpyridin-4-amine (25.6 mg, 0.209 mmol) and 3-(((ethylimino)methylene)amino)-N,N-dimethylpropan-1-amine hydrochloride (80 mg, 0.419 mmol), and the resulting mixture was stirred for 0.5 h at ice-water bath temperature. Then, 2-ethylbutyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.209 mmol) was added, and the resulting mixture was stirred at 25° C. for 4 h. LCMS showed the reaction was completed. The reaction was concentrated in vacuum to give the residue. The residue was dissolved in MeOH (3 mL) and purified by reverse flash chromatography (Biotage, 0%-100% CH3CN/10 mM aq. NH4HCO3; flow rate: 30 mL/min; column: Boston C18, 40 g) to give (2R,3S,5R)-5-(6-amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl dodecanoate (100 mg, 0.108 mmol, 51.6% yield) as a white solid. LCMS (M+H)=899.4; Retention time (0.05% TFA)=2.389 min. ¹H NMR (400 MHz, CDCl₃) δ 7.99 (s, 1H), 7.29 (d, J=2.1 Hz, 1H), 7.25 (s, 1H), 7.14 (dd, J=15.4, 7.9 Hz, 3H), 6.72-6.57 (m, 3H), 6.40 (t, J=6.6 Hz, 1H), 5.93 (s, 2H), 5.62 (dd, J=7.1, 4.6 Hz, 1H), 4.33 (td, J=11.1, 4.9 Hz, 2H), 4.24 (dd, J=11.0, 5.8 Hz, 1H), 4.04-3.92 (m, 2H), 3.77 (t, J=10.3 Hz, 1H), 2.99 (d, J=6.1 Hz, 2H), 2.77 (dt, J=13.9, 7.0 Hz, 1H), 2.71-2.59 (m, 2H), 2.40 (t, J=7.6 Hz, 2H), 1.67 (d, J=7.1 Hz, 2H), 1.43 (dt, J=12.6, 6.3 Hz, 1H), 1.36-1.21 (m, 20H), 0.92-0.78 (m, 9H).

### Example 127

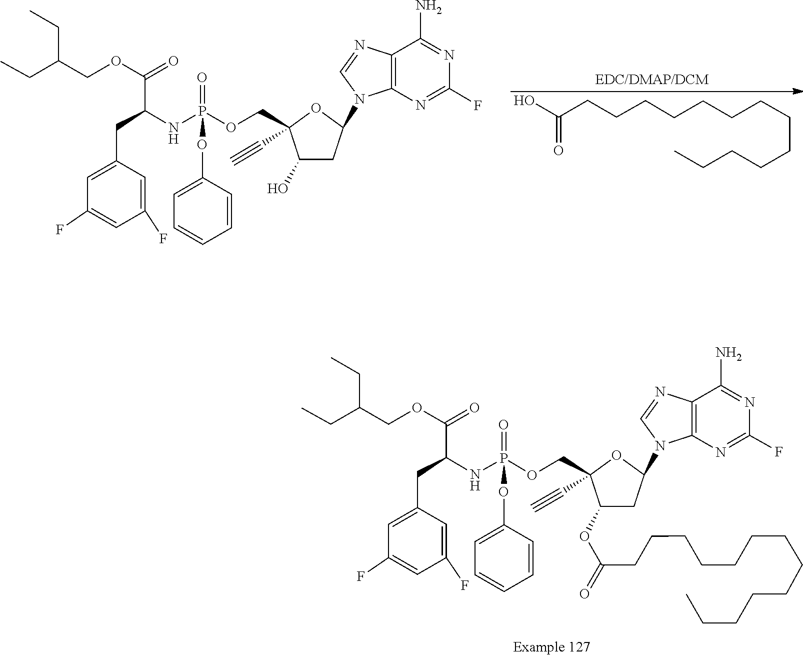 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1O ; CCCCCCCCCCCCCC(=O)O ; C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCCCC]

### (2R,3S,5R)-5-(6-Amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl tetradecanoate

To a solution of tetradecanoic acid (62.1 mg, 0.272 mmol) in DCM (10 mL) was added N,N-dimethylpyridin-4-amine (25.6 mg, 0.209 mmol) and 3-(((ethylimino)methylene)amino)-N,N-dimethylpropan-1-amine hydrochloride (80 mg, 0.419 mmol), and the resulting mixture was stirred for 0.5 h at ice-water bath temperature. Then, 2-ethylbutyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.209 mmol) was added, and the resulting mixture was stirred at 25° C. for 4 h. LCMS showed the reaction was completed. The reaction was concentrated in vacuum to give the residue. The residue was dissolved in MeOH (3 mL) and purified by reverse flash chromatography (Biotage, 0%-100% CH3CN/10 mM aq. NH4HCO3; flow rate: 30 mL/min; column: Boston C18, 40 g) to give (2R,3S,5R)-5-(6-amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl tetradecanoate (110 mg, 0.117 mmol, 56.0% yield) as a white solid. LCMS (M+H)=927.4; Retention time (0.05% TFA)=2.640 min. ¹H NMR (400 MHz, CDCl₃) δ 7.99 (s, 1H), 7.29 (s, 1H), 7.25 (s, 1H), 7.14 (dd, J=15.6, 7.9 Hz, 3H), 6.76-6.56 (m, 3H), 6.40 (t, J=6.6 Hz, 1H), 5.96 (s, 2H), 5.63 (dd, J=7.1, 4.7 Hz, 1H), 4.39-4.29 (m, 2H), 4.29-4.17 (m, 1H), 4.02-3.89 (m, 2H), 3.80 (t, J=10.3 Hz, 1H), 2.99 (d, J=6.1 Hz, 2H), 2.77 (dt, J=13.9, 7.0 Hz, 1H), 2.70-2.58 (m, 2H), 2.40 (t, J=7.6 Hz, 2H), 1.67-1.60 (m, 2H), 1.43 (dt, J=12.5, 6.3 Hz, 1H), 1.37-1.19 (m, 24H), 0.91-0.79 (m, 9H).

### Example 128

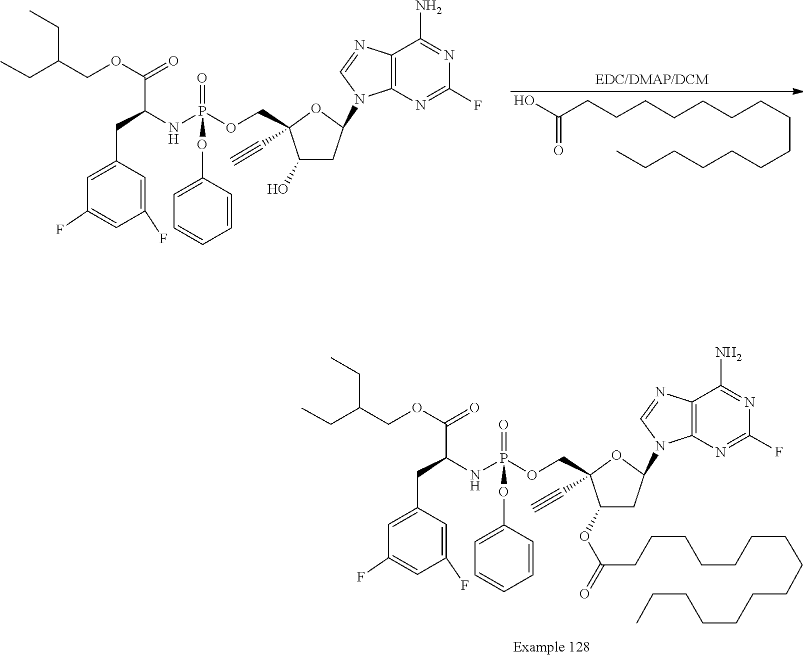 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1O ; CCCCCCCCCCCCCCCC(=O)O ; C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCCCCCC]

### (2R,3S,5R)-5-(6-Amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl palmitate

To a solution of palmitic acid (69.8 mg, 0.272 mmol) in DCM (10 mL) was added N,N-dimethylpyridin-4-amine (25.6 mg, 0.209 mmol), and 3-(((ethylimino)methylene)amino)-N,N-dimethylpropan-1-amine hydrochloride (80 mg, 0.419 mmol), and the resulting mixture was stirred for 0.5 h at ice-water bath temperature. Then, 2-ethylbutyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.209 mmol) was added, and the resulting mixture was stirred at 25° C. for 4 h. LCMS showed the reaction was completed. The reaction was concentrated in vacuum to give the residue. The residue was dissolved in MeOH (3 mL) and purified by reverse flash chromatography (Biotage, 0%-100% CH3CN/10 mM aq. NH4HCO3; flow rate: 30 mL/min; column: Boston C18, 40 g) to give (2R,3S,5R)-5-(6-amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl palmitate (105 mg, 0.108 mmol, 51.7% yield) as a white solid. LCMS: Retention time (0.05% TFA)=3.623 min; HPLC: Retention time (10 mMNH4HCO3)=12.630 min, purity: 98.5%. ¹H NMR (400 MHz, CDCl₃) δ 7.98 (s, 1H), 7.29 (d, J=0.9 Hz, 1H), 7.24 (d, J=4.5 Hz, 1H), 7.14 (dd, J=15.1, 7.8 Hz, 3H), 6.74-6.58 (m, 3H), 6.40 (t, J=6.6 Hz, 1H), 5.86 (s, 2H), 5.63 (dd, J=7.0, 4.6 Hz, 1H), 4.42-4.28 (m, 2H), 4.28-4.20 (m, 1H), 4.04-3.91 (m, 2H), 3.75 (t, J=10.3 Hz, 1H), 2.99 (d, J=6.2 Hz, 2H), 2.78 (dt, J=13.9, 7.1 Hz, 1H), 2.70-2.59 (m, 2H), 2.40 (t, J=7.6 Hz, 2H), 1.67 (d, J=6.9 Hz, 2H), 1.43 (dt, J=12.6, 6.4 Hz, 1H), 1.36-1.20 (m, 28H), 0.86 (dt, J=7.7, 6.8 Hz, 9H).

### Example 129

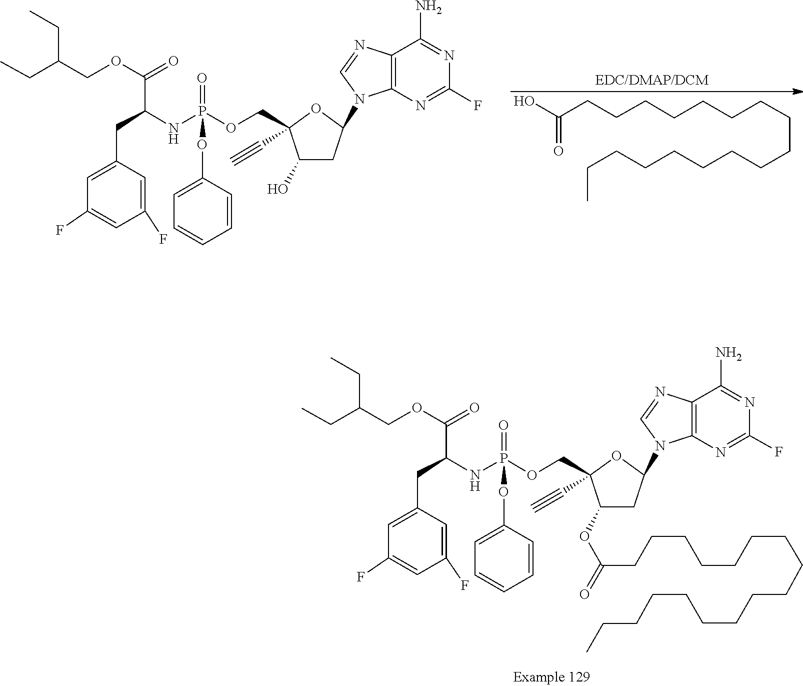 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1O ; CCCCCCCCCCCCCCCCCC(=O)O ; C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCCCCCCCC]

### (2R,3S,5R)-5-(6-Amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl stearate

To a solution of stearic acid (77 mg, 0.272 mmol) in DCM (10 mL) was added N,N-dimethylpyridin-4-amine (25.6 mg, 0.209 mmol) and 3-(((ethylimino)methylene)amino)-N,N-dimethylpropan-1-amine hydrochloride (80 mg, 0.419 mmol), and the resulting mixture was stirred for 0.5 h at ice-water bath temperature. Then, 2-ethylbutyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.209 mmol) was added, and the resulting mixture was stirred at 25° C. for 4 h. LCMS showed the reaction was completed. The reaction was concentrated in vacuum to give the residue. The residue was dissolved in MeOH (3 mL) and purified by reverse flash chromatography (Biotage, 0%-100% CH3CN/10 mM aq. NH4HCO3; flow rate: 30 mL/min; column: Boston C18, 40 g) to give (2R,3S,5R)-5-(6-amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl stearate (105 mg, 0.103 mmol, 49.3% yield) as a white solid. LCMS: Retention time (0.05% TFA)=3.999 min. HPLC: Retention time (10 mMNH4HCO3)=7.697 min. ¹H NMR (400 MHz, CDCl₃) δ 7.99 (s, 1H), 7.29 (s, 1H), 7.24 (d, J=5.6 Hz, 1H), 7.14 (dd, J=15.3, 7.8 Hz, 3H), 6.77-6.53 (m, 3H), 6.40 (t, J=6.6 Hz, 1H), 5.98 (s, 2H), 5.63 (dd, J=7.1, 4.7 Hz, 1H), 4.42-4.29 (m, 2H), 4.28-4.19 (m, 1H), 4.04-3.91 (m, 2H), 3.81 (t, J=10.3 Hz, 1H), 2.99 (d, J=6.2 Hz, 2H), 2.77 (dt, J=13.9, 7.0 Hz, 1H), 2.70-2.58 (m, 2H), 2.40 (t, J=7.6 Hz, 2H), 1.68-1.60 (m, 2H), 1.43 (dt, J=12.5, 6.2 Hz, 1H), 1.38-1.05 (m, 32H), 0.92-0.77 (m, 9H).

### Example 130

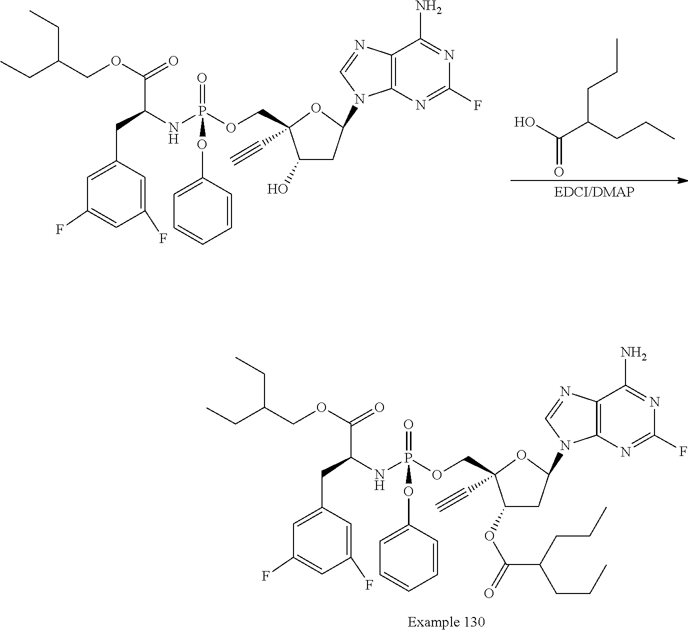

### (2R,3S,5R)-5-(6-Amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl 2-propylpentanoate

To a solution of 2-propylpentanoic acid (39.2 mg, 0.272 mmol) in DCM (10 mL) was added N,N-dimethylpyridin-4-amine (25.6 mg, 0.209 mmol) and 3-(((ethylimino)methylene)amino)-N,N-dimethylpropan-1-amine hydrochloride (80 mg, 0.419 mmol), and the resulting mixture was stirred for 0.5 h at ice-water bath temperature. Then, 2-ethylbutyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.209 mmol) was added, and the resulting mixture was stirred at 25° C. for 4 h. LCMS showed the reaction was completed. The reaction was concentrated in vacuum to give the residue. The residue was dissolved in MeOH (3 mL) and purified by reverse flash chromatography (Biotage, 0%-100% CH3CN/10 mM aq. NH4HCO3; flow rate: 30 mL/min; column: Boston C18, 40 g) to give (2R,3S,5R)-5-(6-amino-2-fluoro-9H-purin-9-yl)-2-((((S)-(((S)-3-(3,5-difluorophenyl)-1-(2-ethylbutoxy)-1-oxopropan-2-yl)amino)(phenoxy)phosphoryl)oxy)methyl)-2-ethynyltetrahydrofuran-3-yl 2-propylpentanoate (90 mg, 0.107 mmol, 51.0% yield) as a white solid. LCMS (M+H)=843.3; Retention time (0.1% TFA)=2.395 min. ¹H NMR (400 MHz, CDCl₃) δ 8.00 (s, 1H), 7.25 (d, J=8.4 Hz, 2H), 7.13 (dd, J=15.4, 7.8 Hz, 3H), 6.64 (ddd, J=11.2, 9.7, 4.1 Hz, 3H), 6.39 (t, J=6.4 Hz, 1H), 6.22 (s, 2H), 5.62 (dd, J=7.1, 5.5 Hz, 1H), 4.41-4.21 (m, 3H), 4.05-3.89 (m, 3H), 2.99 (d, J=6.2 Hz, 2H), 2.83 (dt, J=13.6, 7.0 Hz, 1H), 2.72-2.59 (m, 2H), 2.47 (ddd, J=8.5, 5.4, 3.0 Hz, 1H), 1.66 (ddd, J=20.6, 12.3, 7.1 Hz, 2H), 1.53-1.39 (m, 3H), 1.37-1.20 (m, 8H), 0.91 (td, J=7.1, 1.1 Hz, 6H), 0.83 (t, J=7.4 Hz, 6H).

### Example 130

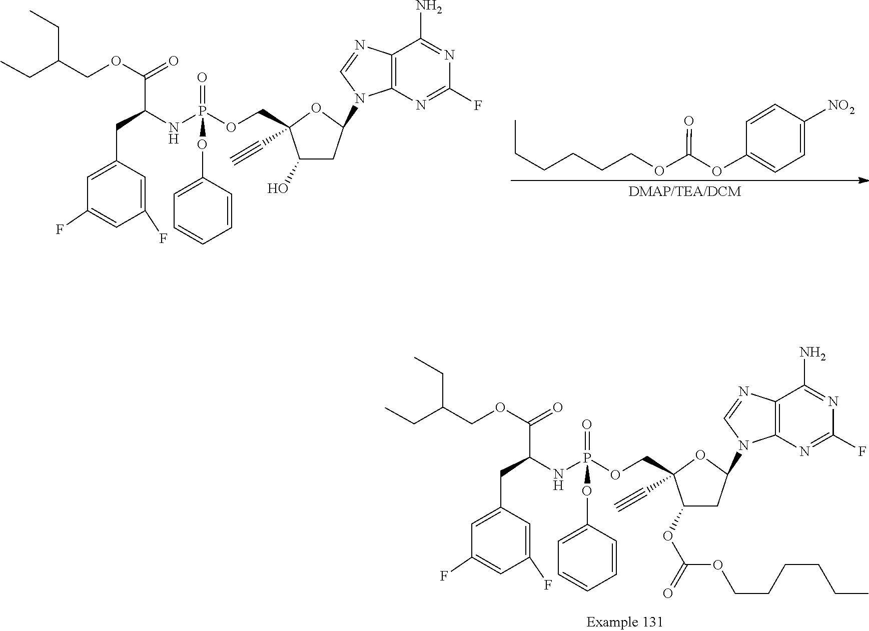

### 2-Ethylbutyl (S)-2-(((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)amino)-3-(3,5-difluorophenyl)propanoate

A mixture of 2-ethylbutyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.209 mmol) in DCM (10 mL) was treated with triethylamine (0.088 mL, 0.628 mmol), DMAP (25.6 mg, 0.209 mmol) followed by hexyl (4-nitrophenyl) carbonate (224 mg, 0.837 mmol), and the mixture was stirred at RT for 2 days. LCMS showed the reaction was completed. The reaction mixture was concentrated in vacuum to give the residue. The residue was dissolved in MeOH (3 mL) and purified by reverse flash chromatography (Biotage, gradient: 70%-100% ACN/10 mM NH₄HCO₃; flow rate: 30 mL/min; column: Boston C18, 40 g) to afford 2-ethylbutyl (S)-2-(((S)-(((2R,3S,5R)-5-(6-amino-2-fluoro-9H-purin-9-yl)-2-ethynyl-3-(((hexyloxy)carbonyl)oxy)tetrahydrofuran-2-yl)methoxy)(phenoxy)phosphoryl)amino)-3-(3,5-difluorophenyl)propanoate (40 mg, 0.047 mmol, 22.62% yield) as white solid. LCMS (M+H)=845.7; Retention time (0.1% TFA)=2.02 min. ¹H NMR (400 MHz, CDCl₃) δ 7.94 (s, 1H), 7.24 (s, 2H), 7.13 (dd, J=13.9, 7.4 Hz, 3H), 6.73-6.60 (m, 3H), 6.38 (t, J=6.5 Hz, 1H), 5.80 (s, 2H), 5.55 (dd, J=7.2, 4.8 Hz, 1H), 4.44-4.24 (m, 3H), 4.19 (t, J=6.3 Hz, 2H), 4.02-3.91 (m, 2H), 3.73 (s, 1H), 2.99 (d, J=6.1 Hz, 2H), 2.92 (dt, J=13.9, 7.0 Hz, 1H), 2.77-2.70 (m, 1H), 2.69 (s, 1H), 1.76-1.65 (m, 2H), 1.46-1.18 (m, 11H), 0.87 (dt, J=14.8, 7.1 Hz, 9H).

### Example 132

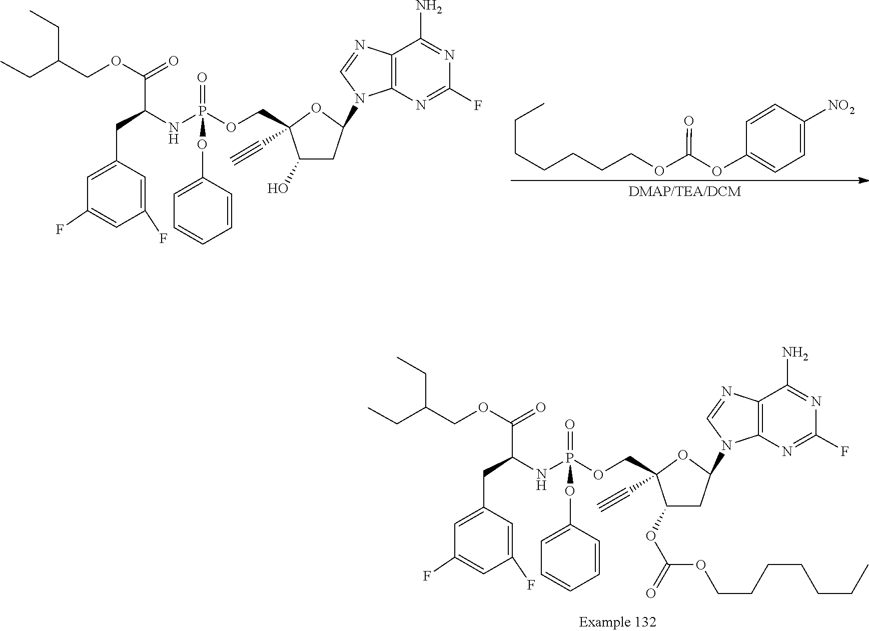

### 2-Ethylbutyl (S)-2-(((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)amino)-3-(3,5-difluorophenyl)propanoate

A mixture of 2-ethylbutyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.209 mmol) in DCM (10 mL) was treated with triethylamine (0.088 mL, 0.628 mmol), DMAP (25.6 mg, 0.209 mmol) followed by heptyl (4-nitrophenyl) carbonate (236 mg, 0.837 mmol), and the mixture was stirred at RT for 2 days. LCMS showed the reaction was completed. The reaction mixture was concentrated in vacuum to give the residue. The residue was dissolved in MeOH (3 mL) and purified by reverse flash chromatography (Biotage, gradient: 70%-100% ACN/10 mM aq. NH₄HCO₃; flow rate: 30 mL/min; column: Boston C18, 40 g) to afford 2-ethylbutyl (S)-2-(((S)-(((2R,3S,5R)-5-(6-amino-2-fluoro-9H-purin-9-yl)-2-ethynyl-3-(((heptyloxy)carbonyl)oxy)tetrahydrofuran-2-yl)methoxy)(phenoxy)phosphoryl)amino)-3-(3,5-difluorophenyl)propanoate (90 mg, 0.105 mmol, 50.1% yield) as white solid. LCMS (M+H)=859.3; Retention time (0.05% TFA)=2.079 min. ¹H NMR (400 MHz, CDCl₃) δ 7.94 (s, 1H), 7.28 (t, J=3.7 Hz, 1H), 7.24 (s, 1H), 7.13 (dd, J=14.6, 7.8 Hz, 3H), 6.73-6.59 (m, 3H), 6.38 (t, J=6.6 Hz, 1H), 5.95 (s, 2H), 5.55 (dd, J=7.1, 4.8 Hz, 1H), 4.39 (dt, J=15.4, 7.7 Hz, 1H), 4.34-4.25 (m, 2H), 4.19 (t, J=6.6 Hz, 2H), 4.02-3.91 (m, 2H), 3.83 (t, J=10.3 Hz, 1H), 2.99 (d, J=6.1 Hz, 2H), 2.91 (dt, J=14.0, 7.0 Hz, 1H), 2.77-2.66 (m, 2H), 1.73 (d, J=6.9 Hz, 1H), 1.67 (s, 1H), 1.47-1.14 (m, 13H), 0.86 (dt, J=14.8, 6.9 Hz, 9H).

### Example 133

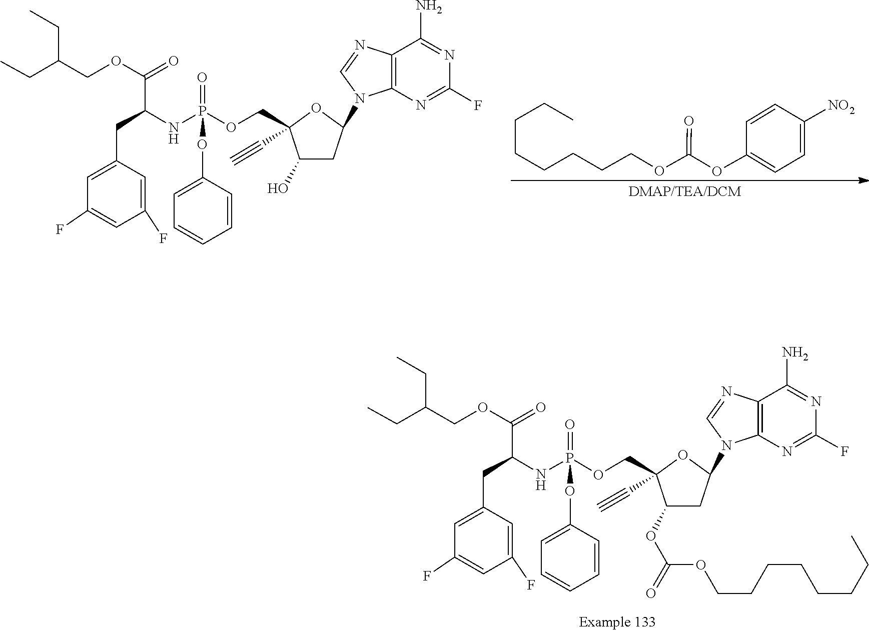

### 2-Ethylbutyl (S)-2-(((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)amino)-3-(3,5-difluorophenyl)propanoate

A mixture of 2-ethylbutyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (150 mg, 0.209 mmol) in DCM (10 mL) was treated with triethylamine (0.088 mL, 0.628 mmol), DMAP (25.6 mg, 0.209 mmol) followed by 4-nitrophenyl octyl carbonate (247 mg, 0.837 mmol), and the mixture was stirred at RT for 2 days. LCMS showed the reaction was completed. The reaction mixture was concentrated in vacuum to give the residue. The residue was dissolved in MeOH (3 mL) and purified by reverse flash chromatography (Biotage, gradient: 70%-100% ACN/10 mM aq. NH₄HCO₃; flow rate: 30 mL/min; column: Boston C18, 40 g) to give 2-ethylbutyl (S)-2-(((S)-(((2R,3S,5R)-5-(6-amino-2-fluoro-9H-purin-9-yl)-2-ethynyl-3-(((octyloxy)carbonyl)oxy)tetrahydrofuran-2-yl)methoxy)(phenoxy)phosphoryl)amino)-3-(3,5-difluorophenyl)propanoate (92 mg, 0.105 mmol, 50.4% yield) as white solid. LCMS (M+H)=873.3; Retention time (0.05% TFA)=2.125 min. ¹H NMR (400 MHz, CDCl₃) δ 7.94 (s, 1H), 7.24 (s, 2H), 7.19-7.07 (m, 3H), 6.77-6.54 (m, 3H), 6.38 (t, J=6.4 Hz, 1H), 6.31-5.89 (m, 2H), 5.59-5.48 (m, 1H), 4.44-4.35 (m, 1H), 4.30 (dd, J=10.8, 5.7 Hz, 2H), 4.19 (t, J=6.6 Hz, 2H), 3.96 (dd, J=13.2, 7.2 Hz, 3H), 2.99 (d, J=6.0 Hz, 2H), 2.89 (d, J=6.8 Hz, 1H), 2.79-2.59 (m, 2H), 1.75-1.58 (m, 2H), 1.34 (ddd, J=19.3, 13.3, 6.5 Hz, 15H), 0.97-0.72 (m, 9H).

### Example 134

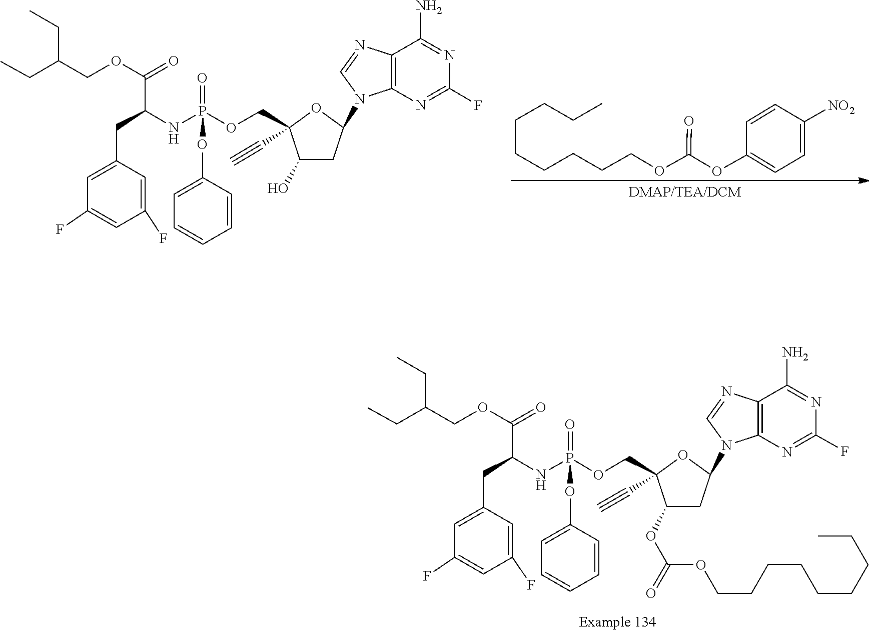

### 2-Ethylbutyl (S)-2-(((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)amino)-3-(3,5-difluorophenyl)propanoate

A mixture of 2-ethylbutyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy) phosphoryl)amino)-3-(3,5-difluorophenyl)propanoate (200 mg, 0.279 mmol) and DMAP (34.1 mg, 0.279 mmol) in DCM (6 mL) was treated with triethylamine (0.117 mL, 0.837 mmol) followed by 4-nitrophenyl nonyl carbonate (259 mg, 0.837 mmol), and the mixture was stirred at 25° C. for 2 days. LCMS showed the reaction was finished. The reaction mixture was concentrated. The residue was dissolved in MeOH (5 mL) and purified by reverse phase chromatography (SepaFlash@ C18 column, Biotage; 0%-100% CH3CN/10 mM aq. NH4HCO3; flow rate: 40 mL/min; column: Boston; 40 g) to give 2-ethylbutyl (S)-2-(((S)-(((2R,3S,5R)-5-(6-amino-2-fluoro-9H-purin-9-yl)-2-ethynyl-3-(((nonyloxy)carbonyl)oxy)tetrahydrofuran-2-yl)methoxy)(phenoxy)phosphoryl)amino)-3-(3,5-difluorophenyl)propanoate (124 mg, 0.139 mmol, 49.6% yield) as a white solid. LCMS (M+H)=887.3; Retention time (0.05% TFA)=2.19 min. ¹H NMR (400 MHz, CDCl₃) δ 8.02 (s, 1H), 7.29-7.24 (m, 2H), 7.17-7.10 (m, 3H), 6.70-6.60 (m, 3H), 6.38 (t, J=6.4 Hz, 1H), 5.94 (brs, 2H), 5.53 (dd, J=7.2, 4.8 Hz, 1H), 4.38 (dd, J=11.2, 6.4 Hz, 1H), 4.35-4.26 (m, 2H), 4.19 (t, J=6.8 Hz, 2H), 4.02-3.92 (m, 2H), 3.75 (t, J=10.0 Hz, 1H), 2.99 (d, J=6.0 Hz, 2H), 2.88 (quint, J=6.8 Hz, 1H), 2.77-2.70 (m, 1H), 2.69 (s, 1H), 1.70-1.66 (m, 2H), 1.43 (quint, J=6.0 Hz, 1H), 1.36-1.21 (m, 16H), 0.88 (t, J=6.8 Hz, 3H), 0.84 (t, J=7.2 Hz, 6H).

### Example 135

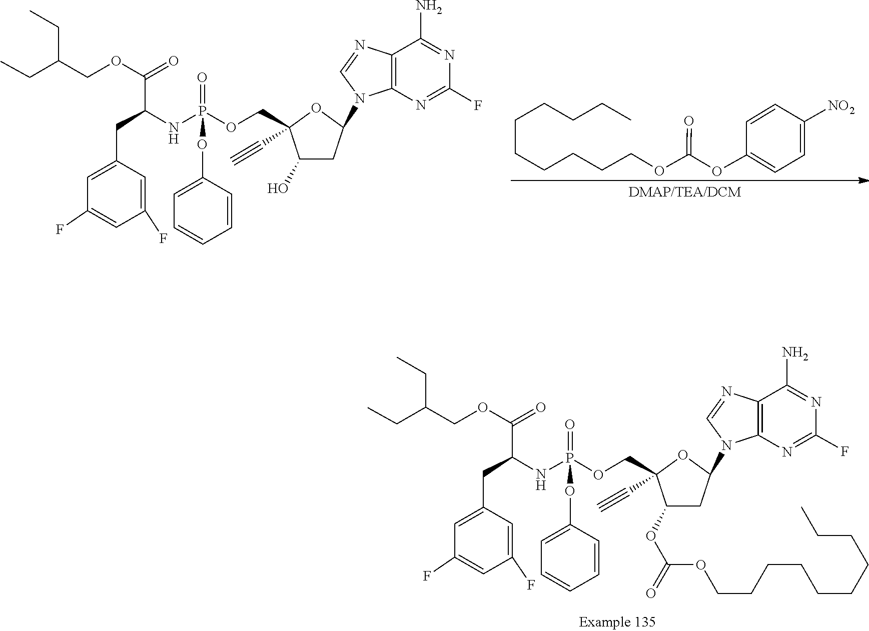

### 2-Ethylbutyl (S)-2-(((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)amino)-3-(3,5-difluorophenyl)propanoate

### (GSK4425554A)

A mixture of 2-ethylbutyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy) phosphoryl)amino)-3-(3,5-difluorophenyl)propanoate (200 mg, 0.279 mmol) and DMAP (34.1 mg, 0.279 mmol) in DCM (6 mL) was treated with triethylamine (0.117 mL, 0.837 mmol) followed by decyl (4-nitrophenyl) carbonate (271 mg, 0.837 mmol), and the mixture was stirred at 25° C. for 2 days. LCMS showed the reaction was finished. The reaction mixture was concentrated. And the residue was dissolved in MeOH (5 mL), and purified by reverse phase chromatography (SepaFlash@ C18 column, Biotage; 0%-100% CH3CN/10 mM aq. NH4HCO3; flow rate: 40 mL/min; column: Boston; 40 g) to give 2-ethylbutyl (S)-2-(((S)-(((2R,3S,5R)-5-(6-amino-2-fluoro-9H-purin-9-yl)-3-(((decyloxy)carbonyl)oxy)-2-ethynyltetrahydrofuran-2-yl)methoxy)(phenoxy)phosphoryl)amino)-3-(3,5-difluorophenyl)propanoate (162 mg, 0.178 mmol, 63.9% yield) as a white solid. LCMS (M+H)=901.1; Retention time (0.05% TFA)=2.27 min. ¹H NMR (400 MHz, CDCl₃) δ 8.01 (s, 1H), 7.29-7.24 (m, 2H), 7.17-7.10 (m, 3H), 6.69-6.66 (m, 2H), 6.65-6.61 (m, 1H), 6.38 (t, J=6.4 Hz, 1H), 5.94 (brs, 2H), 5.53 (dd, J=7.2, 4.8 Hz, 1H), 4.38 (dd, J=11.2, 6.4 Hz, 1H), 4.35-4.26 (m, 2H), 4.19 (t, J=6.4 Hz, 2H), 4.01-3.92 (m, 2H), 3.76 (t, J=10.0 Hz, 1H), 2.99 (d, J=6.0 Hz, 2H), 2.89 (quint, J=6.8 Hz, 1H), 2.77-2.70 (m, 1H), 2.69 (s, 1H), 1.69-1.65 (m, 2H), 1.43 (quint, J=6.4 Hz, 1H), 1.34-1.21 (m, 18H), 0.88 (t, J=6.8 Hz, 3H), 0.84 (t, J=7.6 Hz, 6H).

### Example 136

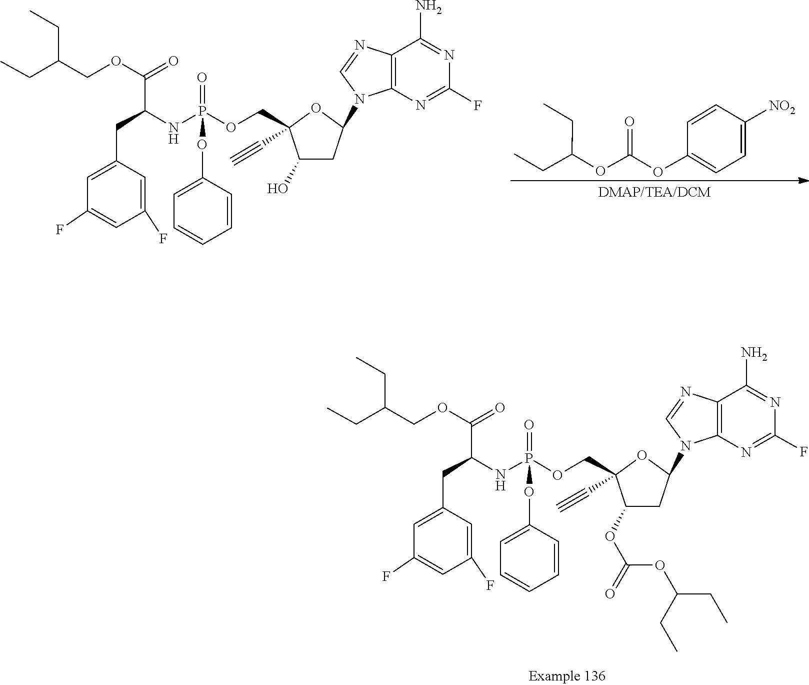 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1O ; CCC(CC)OC(=O)Oc1ccc([N+](=O)[O-])cc1 ; C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OC(CC)CC]

### 2-Ethylbutyl (S)-2-(((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)amino)-3-(3,5-difluorophenyl)propanoate

A mixture of 2-ethylbutyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (200 mg, 0.279 mmol) in DCM (10 mL) was treated with triethylamine (0.117 mL, 0.837 mmol), DMAP (34.1 mg, 0.279 mmol) followed by 4-nitrophenyl pentan-3-yl carbonate (283 mg, 1.116 mmol), and the mixture was stirred at RT for 2 days. TLC showed the reaction was completed. The reaction mixture was concentrated in vacuum to give the residue. The residue was dissolved in MeOH (3 mL) and purified by reverse flash chromatography (Biotage, gradient: 70%-100% ACN/10 mM aq. NH₄HCO₃; flow rate: 30 mL/min; column: Boston C18, 40 g) to afford 2-ethylbutyl (S)-2-(((S)-(((2R,3S,5R)-5-(6-amino-2-fluoro-9H-purin-9-yl)-2-ethynyl-3-(((pentan-3-yloxy)carbonyl)oxy)tetrahydrofuran-2-yl)methoxy)(phenoxy)phosphoryl)amino)-3-(3,5-difluorophenyl)propanoate (100 mg, 0.120 mmol, 43.1% yield) as white solid. LCMS (M+H)=831.7; Retention time (0.1% TFA)=2.264 min. ¹H NMR (400 MHz, CDCl₃) δ 7.95 (s, 1H), 7.24 (s, 2H), 7.13 (dd, J=16.1, 7.9 Hz, 3H), 6.64 (dd, J=22.5, 7.5 Hz, 3H), 6.39 (t, J=6.6 Hz, 1H), 6.14 (s, 2H), 5.53 (dd, J=7.0, 4.3 Hz, 1H), 4.64 (p, J=6.2 Hz, 1H), 4.36 (ddd, J=30.5, 11.0, 6.3 Hz, 3H), 4.06-3.84 (m, 3H), 2.99 (d, J=6.1 Hz, 2H), 2.89 (dt, J=14.0, 7.0 Hz, 1H), 2.80-2.61 (m, 2H), 1.74-1.51 (m, 4H), 1.42 (dt, J=12.3, 6.1 Hz, 1H), 1.35-1.12 (m, 4H), 0.95 (t, J=7.1 Hz, 6H), 0.83 (t, J=7.4 Hz, 6H).

### Example 137

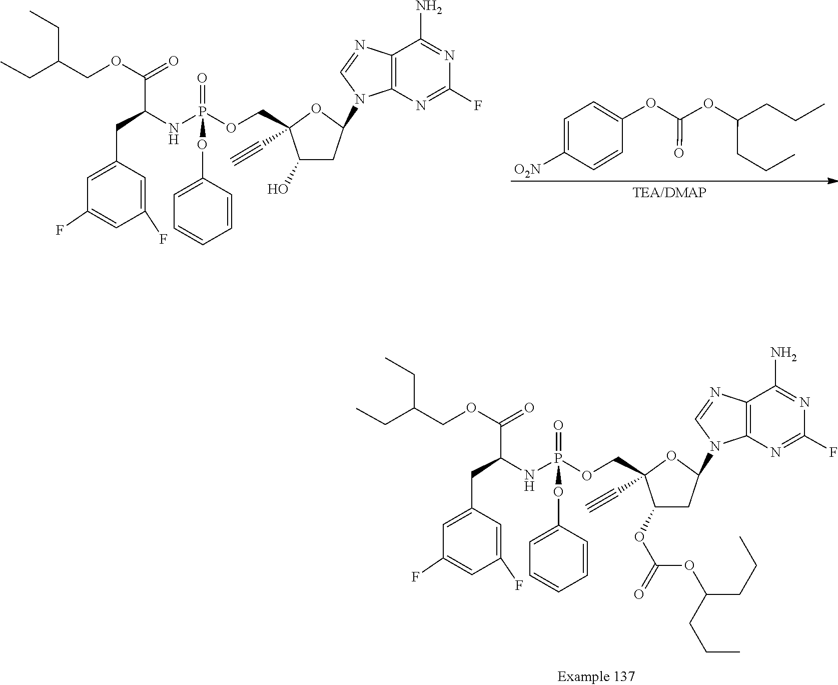 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1O ; CCCC(CCC)OC(=O)Oc1ccc([N+](=O)[O-])cc1 ; C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OC(CCC)CCC]

### 2-Ethylbutyl (S)-2-(((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)amino)-3-(3,5-difluorophenyl)propanoate

### (GSK4422355A)

A mixture of 2-ethylbutyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (200 mg, 0.279 mmol) in DCM (10 mL) was treated with triethylamine (0.077 mL, 0.558 mmol), N,N-dimethylpyridin-4-amine (34.1 mg, 0.279 mmol) followed by heptan-4-yl (4-nitrophenyl) carbonate (314 mg, 1.116 mmol), and the mixture was stirred at RT for 2 days. TLC showed the reaction was completed. The reaction mixture was concentrated in vacuum to give the residue. The crude product was dissolved in DCM (3 mL), loaded to silica column (SanTai, 40 g), and purified by flash (Biotage) chromatography (0-5% MeOH/DCM, 50 mL/min) to afford 2-ethylbutyl (S)-2-(((S)-(((2R,3S,5R)-5-(6-amino-2-fluoro-9H-purin-9-yl)-2-ethynyl-3-(((heptan-4-yloxy)carbonyl)oxy)tetrahydrofuran-2-yl)methoxy)(phenoxy)phosphoryl)amino)-3-(3,5-difluorophenyl)propanoate (120 mg, 0.140 mmol, 50.1% yield) as white solid. LCMS (M+H)=858.7; Retention time (0.1% TFA)=2.635 min. ¹H NMR (400 MHz, CDCl₃) δ 7.95 (s, 1H), 7.24 (s, 2H), 7.18-7.08 (m, 3H), 6.75-6.56 (m, 3H), 6.38 (t, J=6.7 Hz, 1H), 6.27-5.88 (m, 2H), 5.52 (dd, J=7.1, 4.3 Hz, 1H), 4.78 (s, 1H), 4.44-4.25 (m, 3H), 3.97 (dd, J=5.8, 4.0 Hz, 3H), 2.99 (d, J=6.1 Hz, 2H), 2.88 (d, J=7.0 Hz, 1H), 2.66 (s, 2H), 1.69-1.50 (m, 4H), 1.48-1.32 (m, 5H), 1.30-1.18 (m, 4H), 0.93 (td, J=7.3, 5.0 Hz, 6H), 0.83 (t, J=7.4 Hz, 6H).

### Example 138

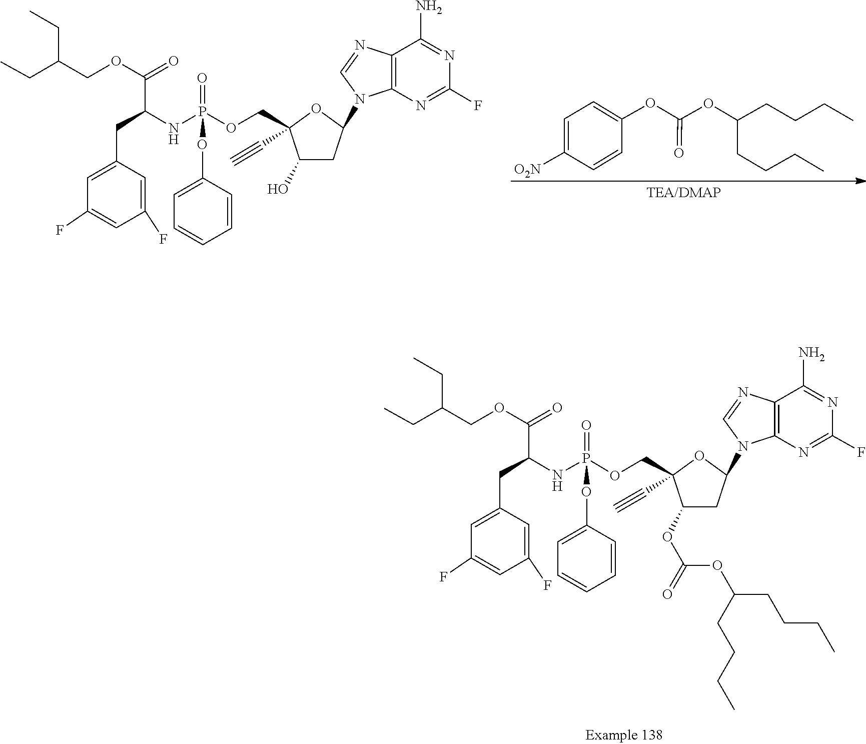 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1O ; CCCCC(CCCC)OC(=O)Oc1ccc([N+](=O)[O-])cc1 ; C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OC(CCCC)CCCC]

### 2-Ethylbutyl (S)-2-(((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)amino)-3-(3,5-difluorophenyl)propanoate

A mixture of 2-ethylbutyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (200 mg, 0.279 mmol) in DCM (10 mL) was treated with triethylamine (0.077 mL, 0.558 mmol), N,N-dimethylpyridin-4-amine (34.1 mg, 0.279 mmol) followed by 4-nitrophenyl nonan-5-yl carbonate (345 mg, 1.116 mmol), and the mixture was stirred at RT for 2 days. TLC showed the reaction was completed. The reaction mixture was concentrated in vacuum to give the residue. The crude product was dissolved in DCM (3 mL), loaded to silica column (SanTai, 40 g), and purified by flash (Biotage) chromatography (0-5% MeOH/DCM, 50 mL/min) to afford 2-ethylbutyl (S)-2-(((S)-(((2R,3S,5R)-5-(6-amino-2-fluoro-9H-purin-9-yl)-2-ethynyl-3-(((nonan-5-yloxy)carbonyl)oxy)tetrahydrofuran-2-yl)methoxy)(phenoxy)phosphoryl)amino)-3-(3,5-difluorophenyl)propanoate (100 mg, 0.113 mmol, 40.4% yield) as white solid. LCMS (M+H)=887.4; Retention time (0.1% TFA)=2.731 min. ¹H NMR (400 MHz, CDCl₃) δ 7.95 (s, 1H), 7.27 (d, J=4.6 Hz, 1H), 7.24 (s, 1H), 7.17-7.09 (m, 3H), 6.66 (dd, J=10.6, 8.7 Hz, 3H), 6.39 (s, 1H), 6.23-5.76 (m, 2H), 5.60-5.46 (m, 1H), 4.82-4.69 (m, 1H), 4.43-4.26 (m, 3H), 4.05-3.77 (m, 3H), 2.99 (d, J=6.1 Hz, 2H), 2.93-2.83 (m, 1H), 2.66 (s, 2H), 1.67-1.53 (m, 4H), 1.40-1.16 (m, 13H), 0.90 (q, J=6.9 Hz, 6H), 0.84 (t, J=7.5 Hz, 6H).

### Example 139

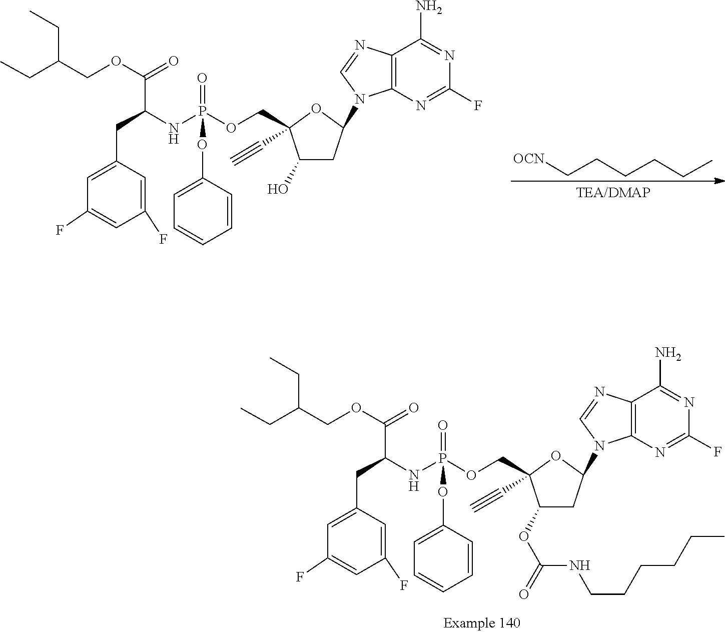

### 2-Ethylbutyl (S)-2-(((S)-(((2R,3S,5R)-5-(6-amino-2-fluoro-9H-purin-9-yl)-2-ethynyl-3-((hexylcarbamoyl)oxy)tetrahydrofuran-2-yl)methoxy)(phenoxy)phosphoryl)amino)-3-(3,5-difluorophenyl)propanoate

A mixture of 2-ethylbutyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (200 mg, 0.279 mmol) and DMAP (68.2 mg, 0.558 mmol) in DCM (5 mL) was treated with TEA (0.117 mL, 0.837 mmol) followed by 1-isocyanatohexane (0.6 mL, 4.12 mmol), and the mixture was stirred at 35° C. for 7 h. LCMS showed the reaction was finished. The reaction mixture was concentrated. The residue was dissolved in DCM (2 mL), loaded to silica gel column (SanTai, 25 g), and purified by flash (Biotage) column chromatography (0-3% MeOH/DCM; flow rate: 30 mL/min) to give 2-ethylbutyl (S)-2-(((S)-(((2R,3S,5R)-5-(6-amino-2-fluoro-9H-purin-9-yl)-2-ethynyl-3-((hexylcarbamoyl)oxy)tetrahydrofuran-2-yl)methoxy)(phenoxy)phosphoryl)amino)-3-(3,5-difluorophenyl)propanoate (96 mg, 0.113 mmol, 40.4% yield) as a white solid. LCMS (M+H)=844.2; Retention time (0.05% TFA)=2.01 min. ¹H NMR (400 MHz, CDCl₃) δ 8.09 (s, 1H), 7.29-7.24 (m, 2H), 7.16 (d, J=8.4 Hz, 2H), 7.11 (t, J=7.2 Hz, 1H), 6.69 (dd, J=7.6, 2.0 Hz, 2H), 6.63 (tt, J=8.8, 2.0 Hz, 1H), 6.38 (t, J=6.4 Hz, 1H), 6.14 (brs, 2H), 5.54 (t, J=6.0 Hz, 1H), 4.89 (t, J=6.0 Hz, 1H), 4.36-4.29 (m, 2H), 4.24 (dd, J=10.8, 6.0 Hz, 1H), 3.96 (dd, J=6.0, 3.6 Hz, 3H), 3.21 (q, J=6.8 Hz, 2H), 3.00 (dd, J=6.0, 3.6 Hz, 2H), 2.70-2.64 (m, 3H), 1.53 (quint, J=6.8 Hz, 2H), 1.42 (quint, J=6.4 Hz, 1H), 1.35-1.21 (m, 10H), 0.89 (t, J=6.8 Hz, 3H), 0.83 (t, J=7.6 Hz, 6H).

1. A compound of formula (I): 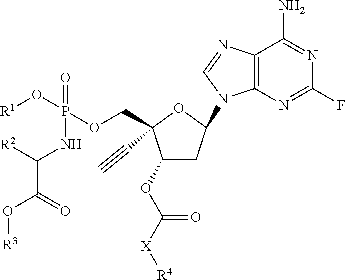
wherein:
R¹ is selected from the group consisting of (C₁-C₂₅) alkyl, (C₁-C₂₅) alkylene-aryl, (C₁-C₂M) alkylene-C₀₂R⁵ and aryl;
R² is selected from the group consisting of (C₁-C₁₀) alkyl; (C₁-C₁₀) alkylene-aryl and aryl;
R³ is selected from the group consisting of (C₁-C₂₅) alkyl, (C₁-C₂₅) alkylene-aryl and aryl; and
R⁴ is selected from the group consisting of (C₁-C₂₅) alkyl, (C₁-C₂₅) alkylene-aryl, (C₁-C₂M) alkylene-C₀₂R⁵ and aryl;
X is a bond, NR⁶, or O
R⁵ is selected from the group consisting of (C₁-C₂₅) alkyl, (C₁-C₂₅) alkylene-aryl and aryl;
R⁶ is H, (C₁-C₂₅) alkyl, (C₁-C₂₅) alkylene-aryl, (C₁-C₂₀) alkylene-C₀₂R⁵ and aryl; and
wherein each of R¹, R², R³, R⁴, R⁵ and R⁶ may be independently and optionally substituted by one or more (C₁-C₁₄) alkyl, Cl, F, oxo, or (C₁-C₆) alkoxy;
or a pharmaceutically acceptable salt thereof.

2. The compound according to claim 1, or a pharmaceutically acceptable salt thereof, wherein R¹ is (C₆-C₁₄)aryl.

3. The compound according to claim 1, or a pharmaceutically acceptable salt thereof, wherein R¹ is C₆ aryl.

4. The compound according to claim 1, or a pharmaceutically acceptable salt thereof, wherein R¹ is (C₁-C₂o) alkylene-CO₂R⁵.

5. The compound according to claim 1, or a pharmaceutically acceptable salt thereof, wherein R¹ is selected from (C₁-C₂₀) alkylene-CO₂R⁵, wherein R⁵ is (C₁-C₂₅) alkyl.

6. The compound according to claim 1, or a pharmaceutically acceptable salt, thereof wherein R² is (C₁-C₁₀)alkylene-(C₆-C₁₄)aryl.

7. The compound according to claim 1, or a pharmaceutically acceptable salt, thereof wherein R² is C₁ alkylene C₆aryl, wherein C₆ aryl is optionally substituted by one or more (C₁-C₁₄) alkyl, Cl, or F.

8. The compound according to claim 1, or a pharmaceutically acceptable salt, thereof wherein R² is (C₁-C₆) alkyl.

9. The compound according to claim 1, or a pharmaceutically acceptable salt thereof wherein R² is (Ci) alkyl.

10. The compound according to claim 1, or a pharmaceutically acceptable salt thereof, wherein R³ is (C₁-C₂₅)alkyl.

11. The compound according to claim 1, or a pharmaceutically acceptable salt thereof, wherein R³ is (C₁-C₆)alkyl.

12. The compound according to claim 1, or a pharmaceutically acceptable salt thereof, wherein R³ is (C₁-C₁₀)alkyl.

13. The compound according to claim 1, or a pharmaceutically acceptable salt thereof, wherein R³ is (C₁₅-C₂₅)alkyl.

14. The compound according to claim 1, or a pharmaceutically acceptable salt thereof, wherein R⁴ is (C₁-C₂₅)alkyl.

15. The compound according to claim 1, or a pharmaceutically acceptable salt thereof, wherein R⁴ is (C₁-C₁₀)alkyl.

16. The compound according to claim 1, or a pharmaceutically acceptable salt thereof, wherein R⁴ is (C₁₅-C₂₀)alkyl.

17. The compound according to claim 1, or a pharmaceutically acceptable salt thereof, wherein X is a bond.

18. The compound according to claim 1, or a pharmaceutically acceptable salt thereof, wherein X is O.

19. The compound according to claim 1, or a pharmaceutically acceptable salt thereof, wherein X is NR⁶.

20. The compound according to claim 1, or a pharmaceutically acceptable salt thereof, wherein R¹ is C₆ aryl, R² is C₁ alkyl or C₁ alkylene C₆aryl, R³ is (C₁-C₂₅)alkyl, R⁴ is (C₁-C₂₅)alkyl and X is a bond.

21. The compound according to claim 1, or a pharmaceutically acceptable salt thereof, wherein R¹ is C₆ aryl, R² is C₁ alkyl or C₁ alkylene C₆aryl, R³ is (C₁-C₂₅)alkyl, R⁴ is (C₁-C₂₅)alkyl and X is O.

22. The compound according to claim 1, or a pharmaceutically acceptable salt thereof, wherein R¹ is C₆ aryl, R² is C₁ alkyl or C₁ alkylene C₆aryl, R³ is (C₁-C₂₅)alkyl, R⁴ is (C₁-C₂₅)alkyl and X is NR⁶.

23. The compound according to claim 1, or a pharmaceutically acceptable salt thereof, wherein R¹ is (C₁-C₂₅)alkylene-CO2-R⁵, R² is selected from Ci alkyl or C₁ alkylene-C₆ aryl, R³ is (C₁-C₂₅)alkyl, R⁴ is (C₁-C₂₅)alkyl and X is a bond.

24. The compound according to claim 1, or a pharmaceutically acceptable salt thereof, wherein R¹ is (C₁-C₂₅)alkylene-CO2-R⁵, R² is selected from Ci alkyl or C₁ alkylene-C₆ aryl, R³ is (C₁-C₂₅)alkyl, R⁴ is (C₁-C₂₅)alkyl and X is O.

25. The compound according to claim 1, or a pharmaceutically acceptable salt thereof, wherein R¹ is (C₁-C₂₅)alkylene-CO₂—R⁵, R² is selected from C₁ alkyl or C₁ alkylene-C₆ aryl, R³ is (C₁-C₂₅)alkyl, R⁴ is (C₁-C₂₅)alkyl and X is NR⁶.

26. A compound selected from the group consisting of:
TABLE 1

| Example | Structure | Chemical Name |
|---|---|---|
| 1 | 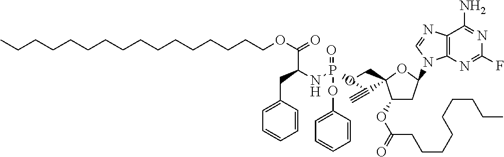 | (2R,3S,5R)-5-(6-Amino-2-fluoro- 9H-purin-9-yl)-2-ethynyl-2- ((((((S)-1-(hexadecyloxy)-1-oxo-3- phenylpropan-2- yl)amino)(phenoxy)phosphoryl)oxy) methyl)tetrahydrofuran-3-yl decanoate |
| 2 | 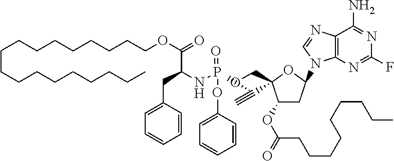 | (2R,3S,5R)-5-(6-Amino-2-fluoro- 9H-purin-9-yl)-2-ethynyl-2- ((((((S)-1-(octadecyloxy)-1-oxo-3- phenylpropan-2- yl)amino)(phenoxy)phosphoryl)oxy) methyl)tetrahydrofuran-3-yl decanoate |
| 3 | 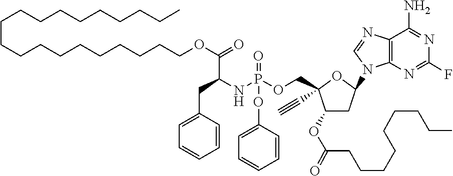 [SMILES: C#C[C@]1(COP(=O)(N[C@@H](Cc2ccccc2)C(=O)OCCCCCCCCCCCCCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2- ((((((S)-1-(icosyloxy)-1-oxo-3- phenylpropan-2- yl)amino)(phenoxy)phosphoryl)oxy) methyl)tetrahydrofuran-3-yl decanoate |
| 4 | 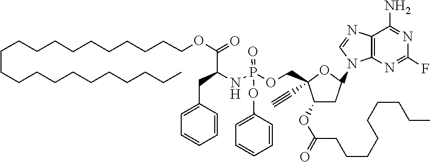 | (2R,3S,5R)-5-(6-amino-2-fluoro- 9H-purin-9-yl)-2-((((((S)-1- (docosyloxy)-1-oxo-3- phenylpropan-2- yl)amino)(phenoxy)phosphoryl)oxy) methyl)-2-ethynyltetrahydrofuran- 3-yl decanoate |
| 5 | 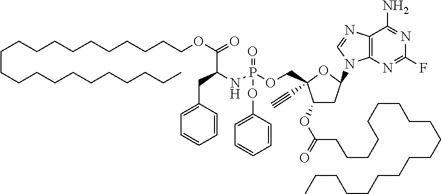 [SMILES: C#C[C@]1(COP(=O)(N[C@@H](Cc2ccccc2)C(=O)OCCCCCCCCCCCCCCCCCCCCCC)Oc2ccccc2)O[C@@H](n2cnc3c(N)nc(F)nc32)C[C@@H]1OC(=O)CCCCCCCCCCCCCCCCCCC] | 2R,3S,5R)-5-(6-amino-2-fluoro- 9H-purin-9-yl)-2-((((((S)-1- (docosyloxy)-1-oxo-3- phenylpropan-2- yl)amino)(phenoxy)phosphoryl)oxy) methyl)-2-ethynyltetrahydrofuran- 3-yl icosanoate |
| 6 | 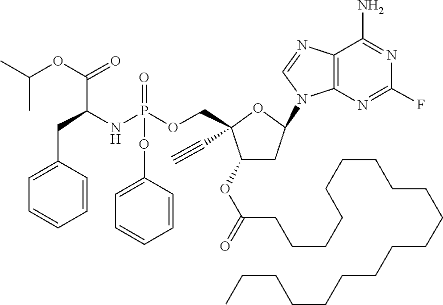 [SMILES: C#C[C@]1(COP(=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2- ((((((S)-1-isopropoxy-1-oxo-3- phenylpropan-2- yl)amino)(phenoxy)phosphoryl)oxy) methyl)tetrahydrofuran-3-yl icosanoate |
| 7 | 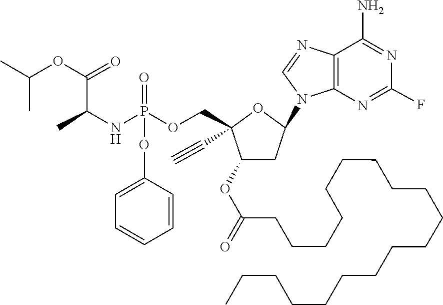 [SMILES: C#C[C@]1(COP(=O)(N[C@@H](C)C(=O)OC(C)C)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2- ((((((S)-1-isopropoxy-1-oxopropan- 2- yl)amino)(phenoxy)phosphoryl)oxy) methyl)tetrahydrofuran-3-yl icosanoate |
| 8 | 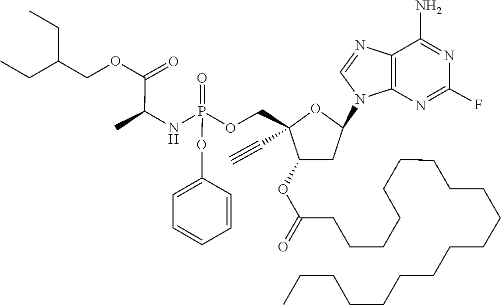 [SMILES: C#C[C@]1(COP(=O)(N[C@@H](C)C(=O)OCC(CC)CC)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-((((((S)-1-(2- ethylbutoxy)-1-oxopropan-2- yl)amino)(phenoxy)phosphoryl)oxy) methyl)-2-ethynyltetrahydrofuran- 3-yl icosanoate |
| 9 | 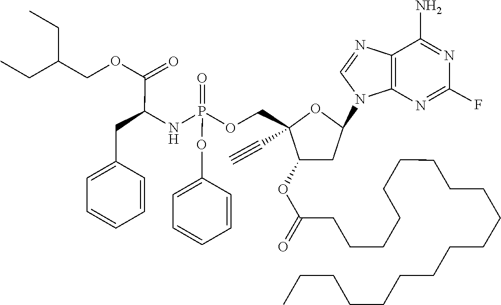 [SMILES: C#C[C@]1(COP(=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-((((((S)-1-(2- ethylbutoxy)-1-oxo-3- phenylpropan-2- yl)amino)(phenoxy)phosphoryl)oxy) methyl)-2-ethynyltetrahydrofuran- 3-yl icosanoate |
| 10 | 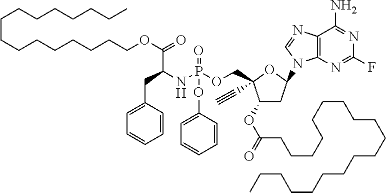 [SMILES: C#C[C@]1(COP(=O)(N[C@@H](Cc2ccccc2)C(=O)OCCCCCCCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2- ((((((S)-1-(hexadecyloxy)-1-oxo-3- phenylpropan-2- yl)amino)(phenoxy)phosphoryl)oxy) methyl)tetrahydrofuran-3-yl icosanoate |
| 11 | 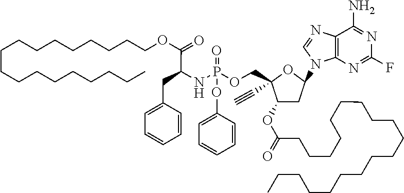 [SMILES: C#C[C@]1(COP(=O)(N[C@@H](Cc2ccccc2)C(=O)OCCCCCCCCCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2- ((((((S)-1-(octadecyloxy)-1-oxo-3- phenylpropan-2- yl)amino)(phenoxy)phosphoryl)oxy) methyl)tetrahydrofuran-3-yl icosanoate |
| 12 | 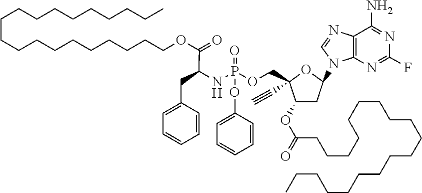 [SMILES: C#C[C@]1(COP(=O)(N[C@@H](Cc2ccccc2)C(=O)OCCCCCCCCCCCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2- ((((((S)-1-(icosyloxy)-1-oxo-3- phenylpropan-2- yl)amino)(phenoxy)phosphoryl)oxy) methyl)tetrahydrofuran-3-yl |
| 13 | 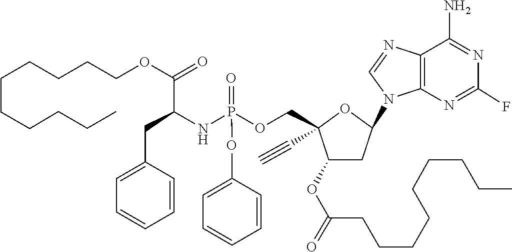 [SMILES: C#C[C@]1(COP(=O)(N[C@@H](Cc2ccccc2)C(=O)OCCCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((((S)-1- (decyloxy)-1-oxo-3-phenylpropan- 2-yl)amino)(phenoxy) phosphoryl)oxy) methyl)-2-ethynyltetrahydrofuran- 3-yl decanoate |
| 14 | 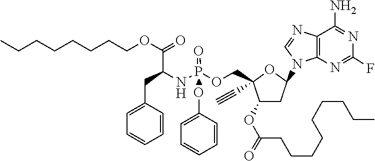 [SMILES: C#C[C@]1(CO[P@@](=O)(N[C@@H](Cc2ccccc2)C(=O)OCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((S)- (((S)-1-(octyloxy)-1-oxo-3- phenylpropan-2- yl)amino)(phenoxy)phosphoryl)oxy) methyl)tetrahydrofuran-3-yl decanoate |
| 15 | 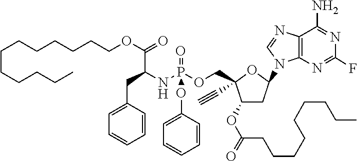 [SMILES: C#C[C@]1(CO[P@@](=O)(N[C@@H](Cc2ccccc2)C(=O)OCCCCCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((S)-(((S)-1- (dodecyloxy)-1-oxo-3- phenylpropan-2- yl)amino)(phenoxy)phosphoryl)oxy) methyl)-2-ethynyltetrahydrofuran- 3-yl decanoate |
| 16 | 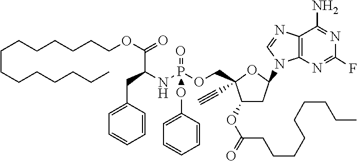 [SMILES: C#C[C@]1(CO[P@@](=O)(N[C@@H](Cc2ccccc2)C(=O)OCCCCCCCCCCCCCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((S)- (((S)-1-oxo-3-phenyl-1- (tetradecyloxy)propan-2- yl)amino)(phenoxy)phosphoryl)oxy) methyl)tetrahydrofuran-3-yl decanoate |
| 17 | 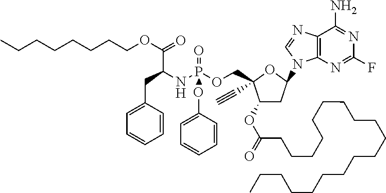 [SMILES: C#C[C@]1(CO[P@@](=O)(N[C@@H](Cc2ccccc2)C(=O)OCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-amino-2-fluoro- 9H-purin-9-yl)-2-ethynyl-2-((((S)- (((S)-1-(octyloxy)-1-oxo-3- phenylpropan-2- yl)amino)(phenoxy)phosphoryl)oxy) methyl)tetrahydrofuran-3-yl icosanoate |
| 18 | 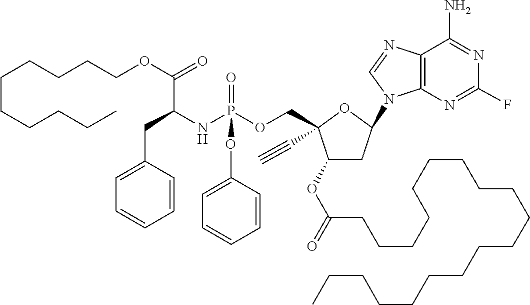 [SMILES: C#C[C@]1(CO[P@@](=O)(N[C@@H](Cc2ccccc2)C(=O)OCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-Amino-2-fluoro- 9H-purin-9-yl)-2-((((S)-(((S)-1- (decyloxy)-1-oxo-3-phenylpropan- 2-yl)amino)(phenoxy) phosphoryl)oxy) methyl)-2-ethynyltetrahydrofuran- 3-yl icosanoate |
| 19 | 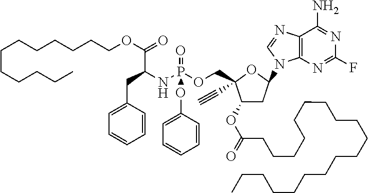 [SMILES: C#C[C@]1(CO[P@@](=O)(N[C@@H](Cc2ccccc2)C(=O)OCCCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-Amino-2-fluoro- 9H-purin-9-yl)-2-((((S)-(((S)-1- (dodecyloxy)-1-oxo-3- phenylpropan-2- yl)amino)(phenoxy)phosphoryl)oxy) methyl)-2-ethynyltetrahydrofuran- 3-yl icosanoate |
| 20 | 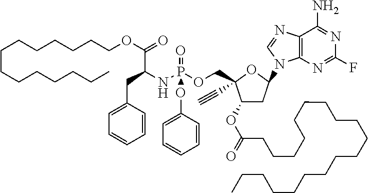 [SMILES: C#C[C@]1(CO[P@@](=O)(N[C@@H](Cc2ccccc2)C(=O)OCCCCCCCCCCCCCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-Amino-2-fluoro- 9H-purin-9-yl)-2-ethynyl-2-((((S)- (((S)-1-oxo-3-phenyl-1- (tetradecyloxy)propan-2- yl)amino)(phenoxy)phosphoryl)oxy) methyl)tetrahydrofuran-3-yl icosanoate |
| 21 | 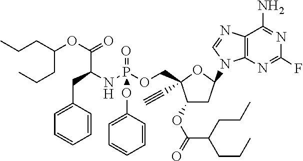 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl 2- propylpentanoate |
| 22 | 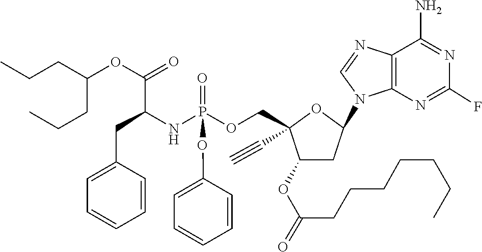 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl octanoate |
| 23 | 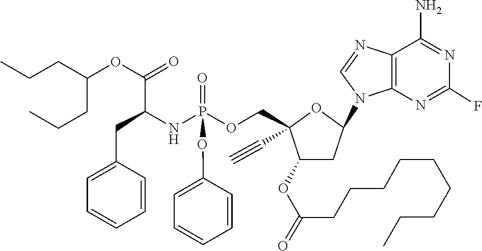 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl decanoate |
| 24 | 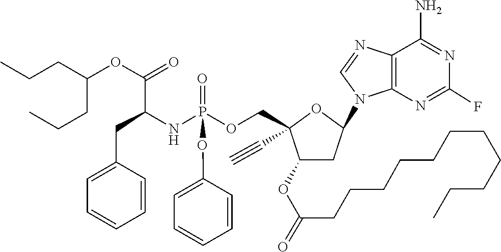 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl dodecanoate |
| 25 | 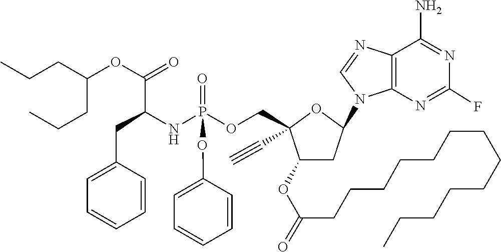 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl tetradecanoate |
| 26 | 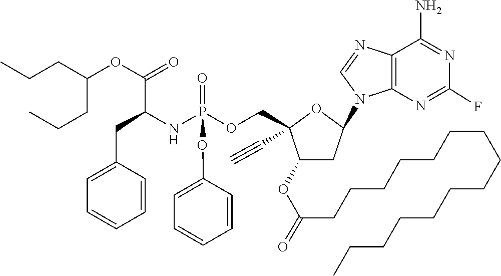 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl palmitate |
| 27 | 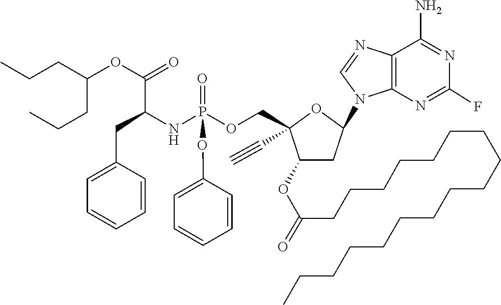 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl stearate |
| 28 | 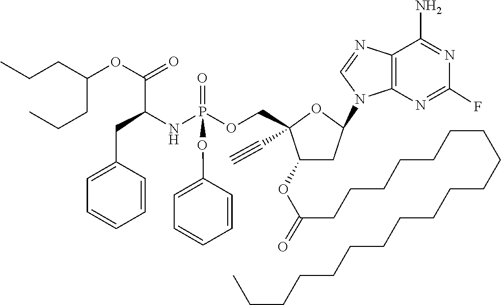 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl icosanoate |
| 29 | 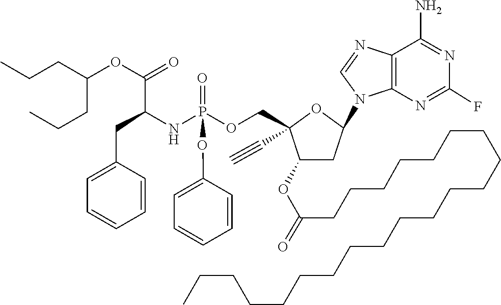 [SMILES: C#C[C@]1(CO[P@@](=O)(N[C@@H](Cc2ccccc2)C(=O)OC(CCC)CCC)Oc2ccccc2)O[C@@H](n2cnc3c(N)nc(F)nc32)C[C@@H]1OC(=O)CCCCCCCCCCCCCCCCCCCCC] | (2R,3S,5R)-5-(6-Amino-2-fluoro- 9H-purin-9-yl)-2-ethynyl-2-((((S)- (((S)-1-(heptan-4-yloxy)-1-oxo-3- phenylpropan-2- yl)amino)(phenoxy)phosphoryl)oxy) methyl)tetrahydrofuran-3-yl docosanoat |
| 30 | 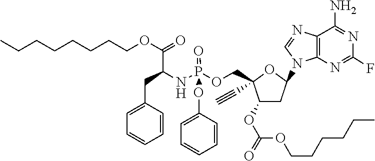 [SMILES: C#C[C@]1(CO[P@@](=O)(N[C@@H](Cc2ccccc2)C(=O)OCCCCCCCC)Oc2ccccc2)O[C@@H](n2cnc3c(N)nc(F)nc32)C[C@@H]1OC(=O)OCCCCCC] | Octyl ((S)-(((2R,3S,5R)-5-(6- amino-2-fluoro-9H-purin-9-yl)-2- ethynyl-3- (((hexyloxy)carbonyl)oxy) tetrahydrofuran-2- yl)methoxy)(phenoxy)phosphoryl)- L-phenylalaninate |
| 31 | 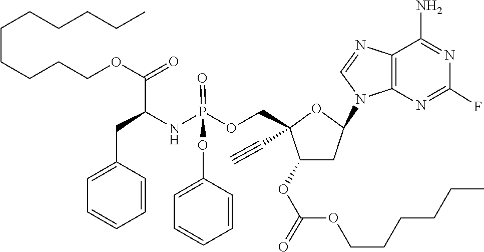 [SMILES: C#C[C@]1(CO[P@@](=O)(N[C@@H](Cc2ccccc2)C(=O)OCCCCCCCCCC)Oc2ccccc2)O[C@@H](n2cnc3c(N)nc(F)nc32)C[C@@H]1OC(=O)OCCCCCC] | Decyl ((S)-(((2R,3S,5R)-5-(6- amino-2-fluoro-9H-purin-9-yl)-2- ethynyl-3- (((hexyloxy)carbonyl)oxy) tetrahydrofuran-2- yl)methoxy)(phenoxy)phosphoryl)- L-phenylalaninate |
| 32 | 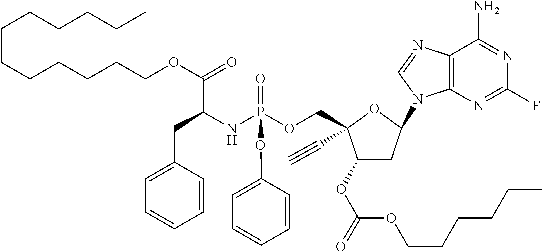 [SMILES: C#C[C@]1(CO[P@@](=O)(N[C@@H](Cc2ccccc2)C(=O)OCCCCCCCCCCCC)Oc2ccccc2)O[C@@H](n2cnc3c(N)nc(F)nc32)C[C@@H]1OC(=O)OCCCCCC] | Dodecyl ((S)-(((2R,3S,5R)-5-(6- amino-2-fluoro-9H-purin-9-yl)-2- ethynyl-3- (((hexyloxy)carbonyl)oxy) tetrahydrofuran-2- yl)methoxy)(phenoxy)phosphoryl)- L-phenylalaninate |
| 33 | 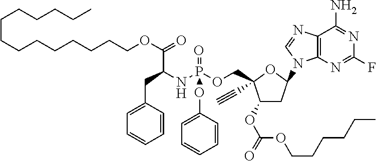 [SMILES: C#C[C@]1(CO[P@@](=O)(N[C@@H](Cc2ccccc2)C(=O)OCCCCCCCCCCCCCC)Oc2ccccc2)O[C@@H](n2cnc3c(N)nc(F)nc32)C[C@@H]1OC(=O)OCCCCCC] | Tetradecyl ((S)-(((2R,3S,5R)-5-(6- amino-2-fluoro-9H-purin-9-yl)-2- ethynyl-3- (((hexyloxy)carbonyl)oxy) tetrahydrofuran-2- yl)methoxy)(phenoxy)phosphoryl)- L-phenylalaninate |
| 34 | 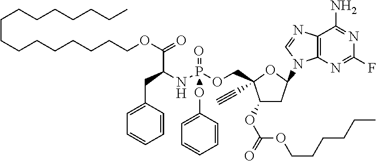 [SMILES: C#C[C@]1(CO[P@@](=O)(N[C@@H](Cc2ccccc2)C(=O)OCCCCCCCCCCCCCCCC)Oc2ccccc2)O[C@@H](n2cnc3c(N)nc(F)nc32)C[C@@H]1OC(=O)OCCCCCC] | Hexadecyl ((S)-(((2R,3S,5R)-5-(6- amino-2-fluoro-9H-purin-9-yl)-2- ethynyl-3- (((hexyloxy)carbonyl)oxy) tetrahydrofuran-2- yl)methoxy)(phenoxy)phosphoryl)- L-phenylalaninate |
| 35 | 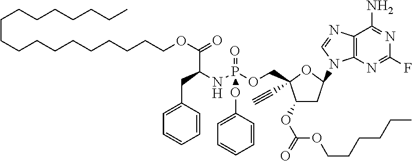 [SMILES: C#C[C@]1(CO[P@@](=O)(N[C@@H](Cc2ccccc2)C(=O)OCCCCCCCCCCCCCCCCCC)Oc2ccccc2)O[C@@H](n2cnc3c(N)nc(F)nc32)C[C@@H]1OC(=O)OCCCCCC] | Octadecyl ((S)-(((2R,3S,5R)-5-(6- amino-2-fluoro-9H-purin-9-yl)-2- ethynyl-3- (((hexyloxy)carbonyl)oxy) tetrahydrofuran-2- yl)methoxy)(phenoxy)phosphoryl)- L-phenylalaninate |
| 36 | 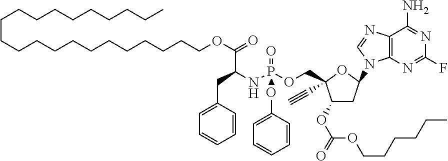 [SMILES: C#C[C@]1(CO[P@@](=O)(N[C@@H](Cc2ccccc2)C(=O)OCCCCCCCCCCCCCCCCCCCCCC)Oc2ccccc2)O[C@@H](n2cnc3c(N)nc(F)nc32)C[C@@H]1OC(=O)OCCCCCC] | Icosyl ((S)-(((2R,3S,5R)-5-(6- amino-2-fluoro-9H-purin-9-yl)-2- ethynyl-3- (((hexyloxy)carbonyl)oxy) tetrahydrofuran-2- yl)methoxy)(phenoxy)phosphoryl)- L-phenylalaninate |
| 37 | 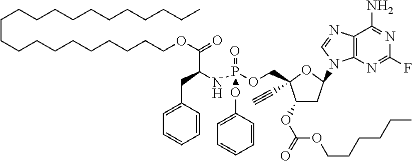 [SMILES: C#C[C@]1(CO[P@@](=O)(N[C@@H](Cc2ccccc2)C(=O)OCCCCCCCCCCCCCCCCCCCCCC)Oc2ccccc2)O[C@@H](n2cnc3c(N)nc(F)nc32)C[C@@H]1OC(=O)OCCCCCC] | Docosyl ((S)-(((2R,3S,5R)-5-(6- amino-2-fluoro-9H-purin-9-yl)-2- ethynyl-3- (((hexyloxy)carbonyl)oxy) tetrahydrofuran-2- yl)methoxy)(phenoxy)phosphoryl)- L-phenylalaninate |
| 38 | 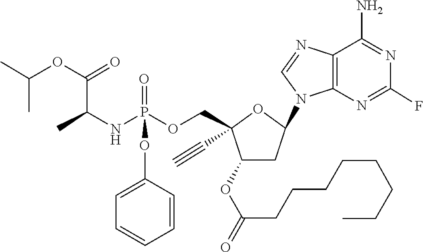 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl nonanoate |
| 39 | 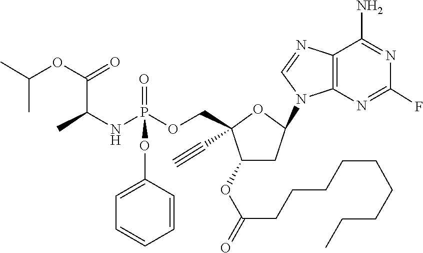 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl decanoate |
| 40 | 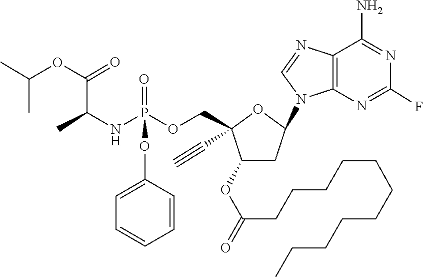 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl dodecanoate |
| 41 | 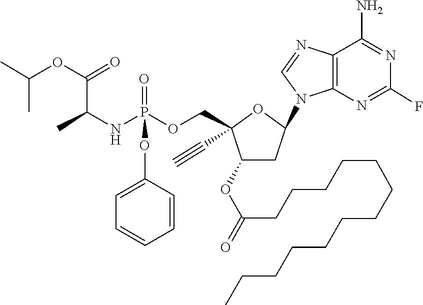 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl tetradecanoate |
| 42 | 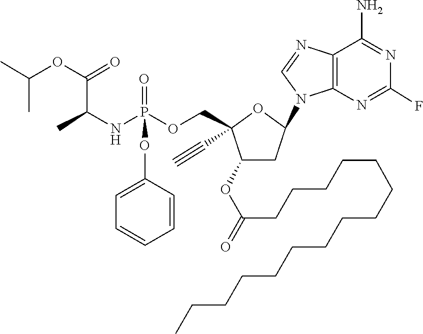 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl palmitate |
| 43 | 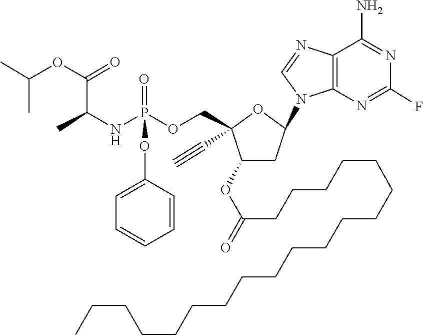 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCCCCCCCCCCCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl stearate |
| 44 | 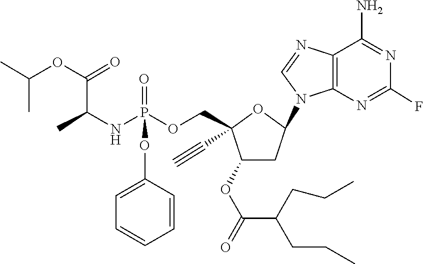 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-ethynyl-2-((((S)- (((S)-1-isopropoxy-1-oxopropan-2- yl)amino)(phenoxy)phosphoryl)oxy) methyl)tetrahydrofuran-3-yl 2- propylpentanoate |
| 45 | 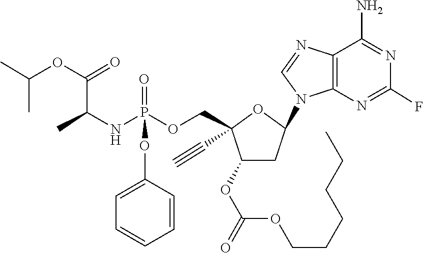 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OCCCCCC] | Isopropyl ((S)-(((2R,3S,5R)-5-(6- amino-2-fluoro-9H-purin-9-yl)-2- ethynyl-3- (((hexyloxy)carbonyl)oxy) tetrahydrofuran-2- yl)methoxy)(phenoxy)phosphoryl)- L-alaninate |
| 46 | 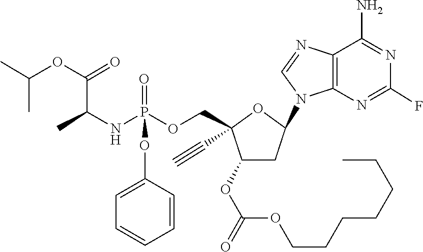 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OCCCCCCC] | Isopropyl ((S)-(((2R,3S,5R)-5-(6- amino-2-fluoro-9H-purin-9-yl)-2- ethynyl-3- (((heptyloxy)carbonyl)oxy) tetrahydrofuran-2- yl)methoxy)(phenoxy)phosphoryl)- L-alaninate |
| 47 | 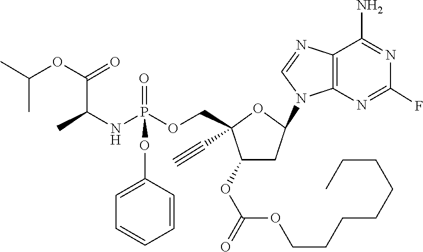 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OCCCCCCCC] | Isopropyl ((S)-(((2R,3S,5R)-5-(6- amino-2-fluoro-9H-purin-9-yl)-2- ethynyl-3- (((octyloxy)carbonyl)oxy) tetrahydrofuran-2- yl)methoxy)(phenoxy)phosphoryl)- L-alaninate |
| 48 | 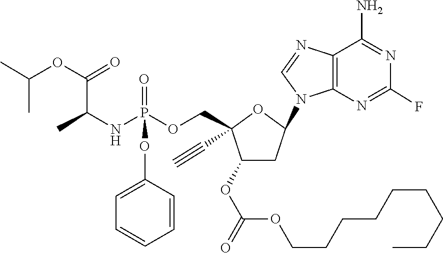 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OCCCCCCCCC] | Isopropyl ((S)-(((2R,3S,5R)-5-(6- amino-2-fluoro-9H-purin-9-yl)-2- ethynyl-3- (((nonyloxy)carbonyl)oxy) tetrahydrofuran-2- yl)methoxy)(phenoxy)phosphoryl)- L-alaninate |
| 49 | 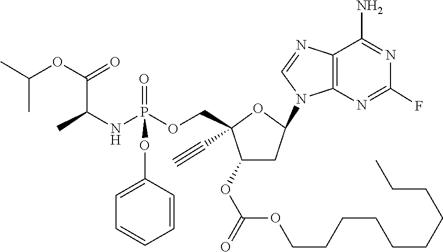 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OCCCCCCCCCC] | Isopropyl ((S)-(((2R,3S,5R)-5-(6- amino-2-fluoro-9H-purin-9-yl)-3- (((decyloxy)carbonyl)oxy)-2- ethynyltetrahydrofuran-2- yl)methoxy)(phenoxy)phosphoryl)- L-alaninate |
| 50 | 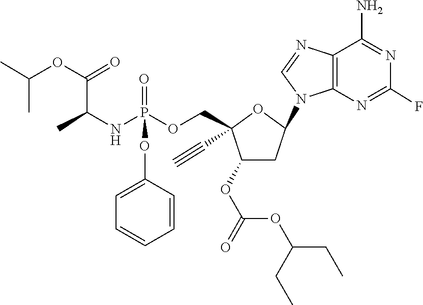 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OC(CC)CC] | Isopropyl ((S)-(((2R,3S,5R)-5-(6- amino-2-fluoro-9H-purin-9-yl)-2- ethynyl-3-(((pentan-3- yloxy)carbonyl)oxy) tetrahydrofuran-2- yl)methoxy)(phenoxy)phosphoryl)- L-alaninate |
| 51 | 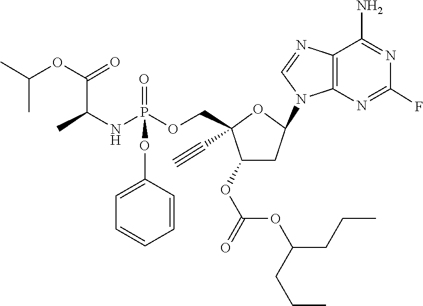 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OC(CCC)CCC] | Isopropyl ((S)-(((2R,3S,5R)-5-(6- amino-2-fluoro-9H-purin-9-yl)-2- ethynyl-3-(((heptan-4- yloxy)carbonyl)oxy) tetrahydrofuran-2- yl)methoxy)(phenoxy)phosphoryl)- L-alaninate |
| 52 | 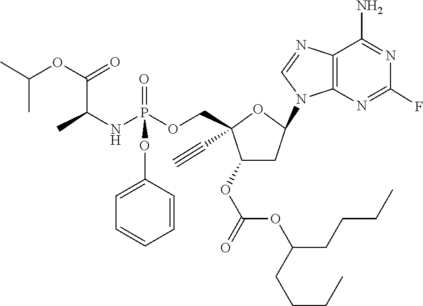 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)OC(CCCC)CCCC] | Isopropyl ((S)-(((2R,3S,5R)-5-(6- amino-2-fluoro-9H-purin-9-yl)-2- ethynyl-3-(((nonan-5- yloxy)carbonyl)oxy) tetrahydrofuran-2- yl)methoxy)(phenoxy)phosphoryl)- L-alaninate |
| 53 | 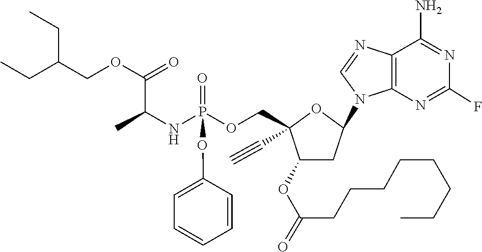 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran- 3-yl nonanoate |
| 54 | 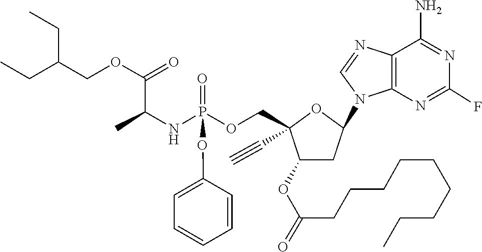 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran- 3-yl decanoate |
| 55 | 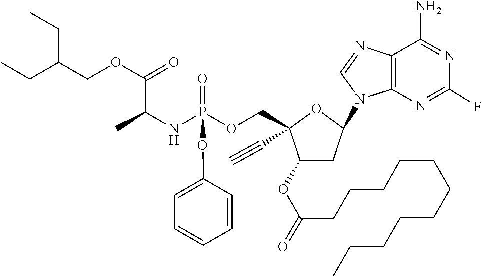 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran- 3-yl dodecanoate |
| 56 | 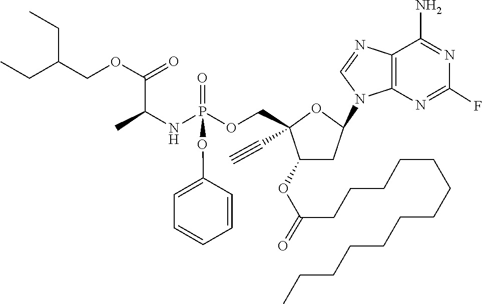 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran- 3-yl tetradecanoate |
| 57 | 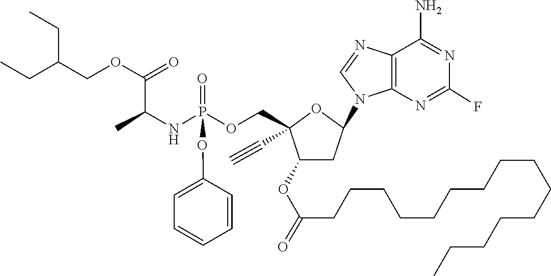 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran- 3-yl palmitate |
| 58 | 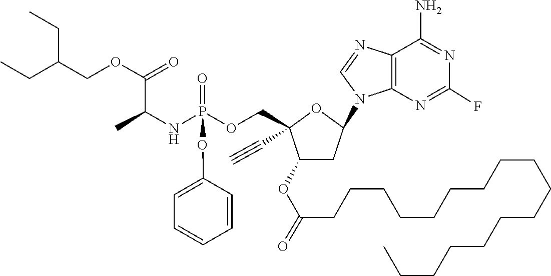 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)CCCCCCCCCCCCCCCCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl)oxy) methyl)-2-ethynyltetrahydrofuran- 3-yl stearate |
| 59 | 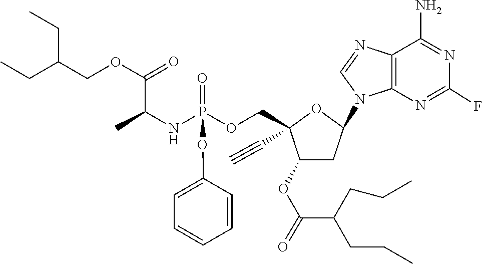 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-((((S)-(((S)-1-(2- ethylbutoxy)-1-oxopropan-2- yl)amino)(phenoxy)phosphoryl) oxy)methyl)-2- ethynyltetrahydrofuran- 3-yl 2-propylpentanoate |
| 60 | 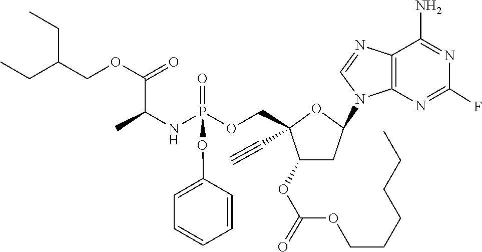 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCC] | 2-Ethylbutyl ((S)-(((2R,3S,5R)-5- (6-amino-2-fluoro-9H-purin-9-yl)- 2-ethynyl-3- (((hexyloxy)carbonyl)oxy) tetrahydrofuran-2- yl)methoxy)(phenoxy)phosphoryl)- L-alaninate |
| 61 | 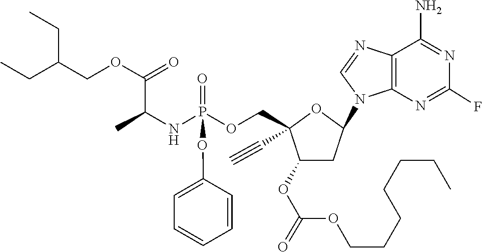 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCC] | 2-Ethylbutyl ((S)-(((2R,3S,5R)-5- (6-amino-2-fluoro-9H-purin-9-yl)- 2-ethynyl-3- (((heptyloxy)carbonyl)oxy) tetrahydrofuran-2- yl)methoxy)(phenoxy)phosphoryl)- L-alaninate |
| 62 | 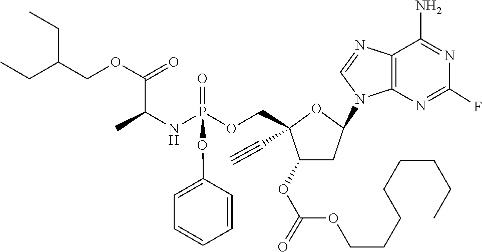 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCCC] | 2-Ethylbutyl ((S)-(((2R,3S,5R)-5- (6-amino-2-fluoro-9H-purin-9-yl)- 2-ethynyl-3- (((octyloxy)carbonyl)oxy) tetrahydrofuran-2- yl)methoxy)(phenoxy)phosphoryl)- L-alaninate |
| 63 | 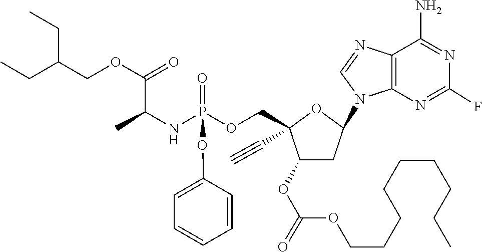 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCCCC] | 2-Ethylbutyl ((S)-(((2R,3S,5R)-5- (6-amino-2-fluoro-9H-purin-9-yl)- 2-ethynyl-3- (((nonyloxy)carbonyl)oxy) tetrahydrofuran-2- yl)methoxy)(phenoxy)phosphoryl)- L-alaninate |
| 64 | 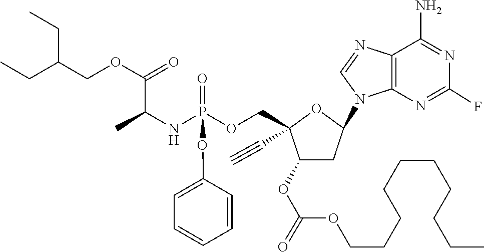 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OCCCCCCCCCC] | 2-Ethylbutyl ((S)-(((2R,3S,5R)-5- (6-amino-2-fluoro-9H-purin-9-yl)- 3-(((decyloxy)carbonyl)oxy)-2- ethynyltetrahydrofuran-2- yl)methoxy)(phenoxy)phosphoryl)- L-alaninate |
| 65 | 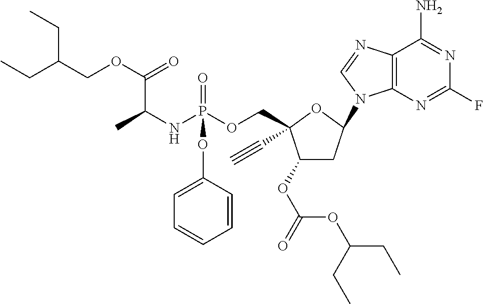 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OC(CC)CC] | 2-Ethylbutyl ((S)-(((2R,3S,5R)-5- (6-amino-2-fluoro-9H-purin-9-yl)- 2-ethynyl-3-(((pentan-3- yloxy)carbonyl)oxy) tetrahydrofuran-2- yl)methoxy)(phenoxy)phosphoryl)- L-alaninate |
| 66 | 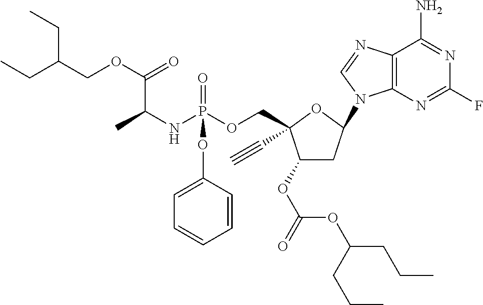 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OC(CCC)CCC] | 2-Ethylbutyl ((S)-(((2R,3S,5R)-5- (6-amino-2-fluoro-9H-purin-9-yl)- 2-ethynyl-3-(((heptan-4- yloxy)carbonyl)oxy) tetrahydrofuran-2- yl)methoxy)(phenoxy)phosphoryl)- L-alaninate |
| 67 | 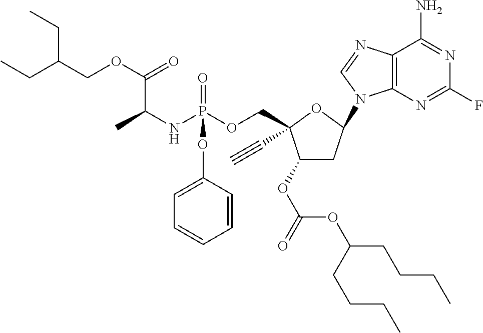 [SMILES: C#C[C@]1(CO[P@@](=O)(N[C@@H](C)C(=O)OCC(CC)CC)Oc2ccccc2)O[C@@H](n2cnc3c(N)nc(F)nc32)C[C@@H]1OC(=O)OC(CCCC)CCCC] | 2-Ethylbutyl ((S)-(((2R,3S,5R)-5- (6-amino-2-fluoro-9H-purin-9-yl)- 2-ethynyl-3-(((nonan-5- yloxy)carbonyl)oxy) tetrahydrofuran-2- yl)methoxy)(phenoxy)phosphoryl)- L-alaninate |
| 68 | 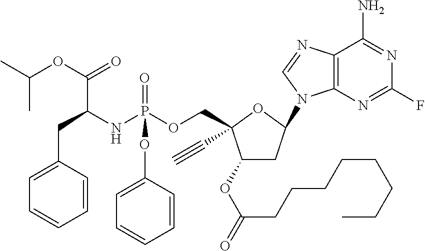 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl) oxy)methyl)tetrahydrofuran-3-yl nonanoate |
| 69 | 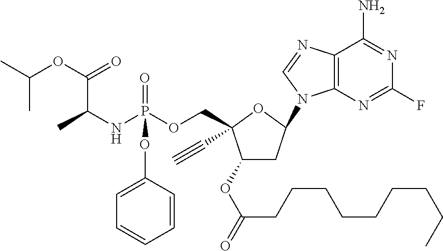 [SMILES: C#C[C@]1(CO[P@@](=O)(N[C@@H](C)C(=O)OC(C)C)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl) oxy)methyl)tetrahydrofuran-3-yl decanoate |
| 70 | 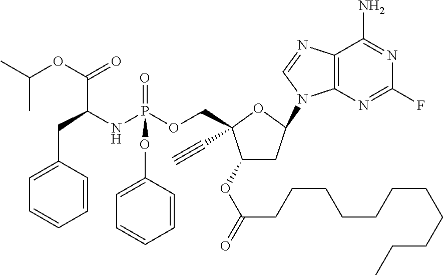 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCC] | (2R,3S,5R)-5-(6-amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl dodecanoate |
| 71 | 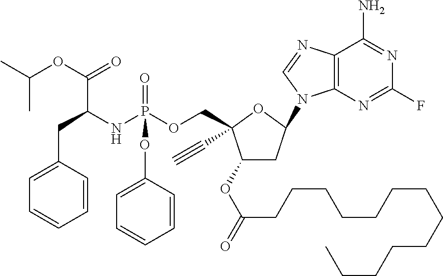 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl tetradecanoate |
| 72 | 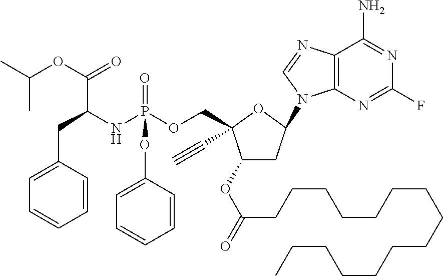 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl palmitate |
| 73 | 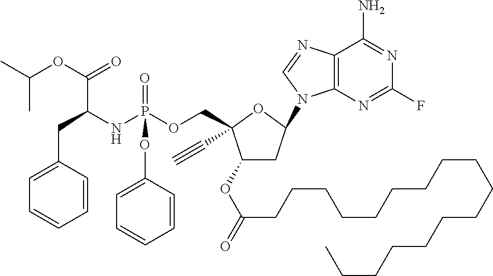 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)CCCCCCCCCCCCCCCCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl stearate |
| 74 | 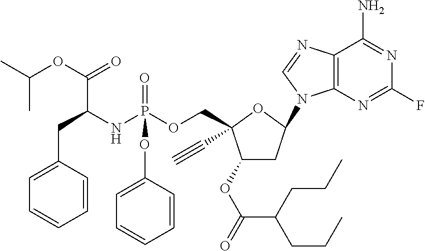 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-ethynyl-2-((((S)- (((S)-1-isopropoxy-1-oxo-3- phenylpropan-2- yl)amino)(phenoxy)phosphoryl)oxy) methyl)tetrahydrofuran-3-yl 2- propylpentanoate |
| 75 | 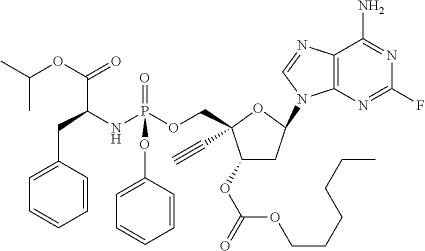 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCC] | Isopropyl ((S)-(((2R,3S,5R)-5-(6- amino-2-fluoro-9H-purin-9-yl)-2- ethynyl-3- (((hexyloxy)carbonyl)oxy) tetrahydrofuran-2- yl)methoxy)(phenoxy)phosphoryl)- L-phenylalaninate |
| 76 | 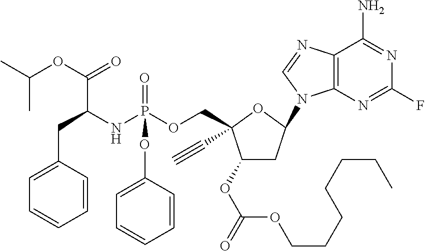 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCC] | Isopropyl ((S)-(((2R,3S,5R)-5-(6- amino-2-fluoro-9H-purin-9-yl)-2- ethynyl-3- (((heptyloxy)carbonyl)oxy) tetrahydrofuran-2- yl)methoxy)(phenoxy)phosphoryl)- L-phenylalaninate |
| 77 | 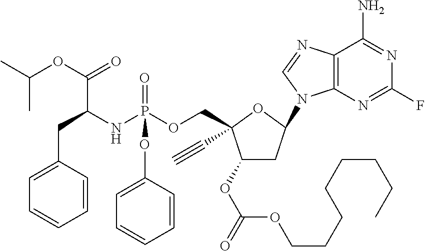 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCCC] | Isopropyl ((S)-(((2R,3S,5R)-5-(6- amino-2-fluoro-9H-purin-9-yl)-2- ethynyl-3- (((octyloxy)carbonyl)oxy) tetrahydrofuran-2- yl)methoxy)(phenoxy)phosphoryl)- L-phenylalaninate |
| 78 | 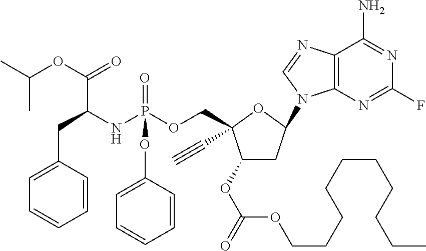 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCCCCC] | Isopropyl ((S)-(((2R,3S,5R)-5-(6- amino-2-fluoro-9H-purin-9-yl)-3- (((decyloxy)carbonyl)oxy)-2- ethynyltetrahydrofuran-2- yl)methoxy)(phenoxy)phosphoryl)- L-phenylalaninat |
| 79 | 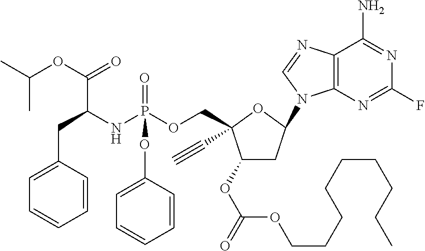 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OCCCCCCCCC] | Isopropyl ((S)-(((2R,3S,5R)-5-(6- amino-2-fluoro-9H-purin-9-yl)-2- ethynyl-3- (((nonyloxy)carbonyl)oxy) tetrahydrofuran-2- yl)methoxy)(phenoxy)phosphoryl)- L-phenylalaninate |
| 80 | 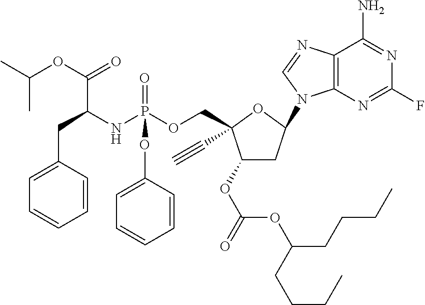 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OC(CCCC)CCCC] | Isopropyl ((S)-(((2R,3S,5R)-5-(6- amino-2-fluoro-9H-purin-9-yl)-2- ethynyl-3-(((nonan-5- yloxy)carbonyl)oxy) tetrahydrofuran-2- yl)methoxy)(phenoxy)phosphoryl)- L-phenylalaninate |
| 81 | 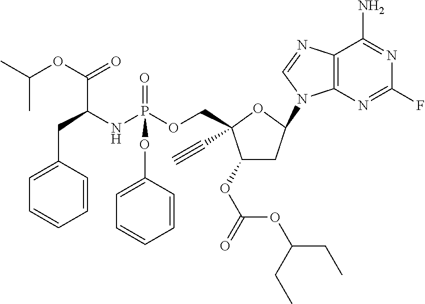 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OC(CC)CC] | Isopropyl ((S)-(((2R,3S,5R)-5-(6- amino-2-fluoro-9H-purin-9-yl)-2- ethynyl-3-(((pentan-3- yloxy)carbonyl)oxy) tetrahydrofuran-2- yl)methoxy)(phenoxy)phosphoryl)- L-phenylalaninate |
| 82 | 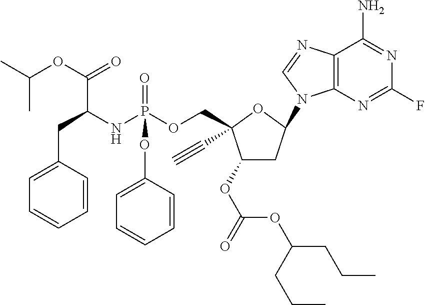 [SMILES: C#C[C@]1(CO[P@@](=O)(N[C@@H](Cc2ccccc2)C(=O)OC(C)C)Oc2ccccc2)O[C@@H](n2cnc3c(N)nc(F)nc32)C[C@@H]1OC(=O)OC(CCC)CCC] | Isopropyl ((S)-(((2R,3S,5R)-5-(6- amino-2-fluoro-9H-purin-9-yl)-2- ethynyl-3-(((heptan-4- yloxy)carbonyl) oxy)tetrahydrofuran-2- yl)methoxy)(phenoxy)phosphoryl)- L-phenylalaninate |
| 83 | 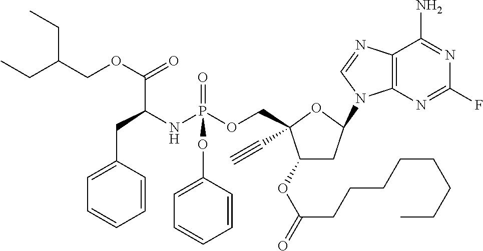 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran- 3-yl nonanoate |
| 84 | 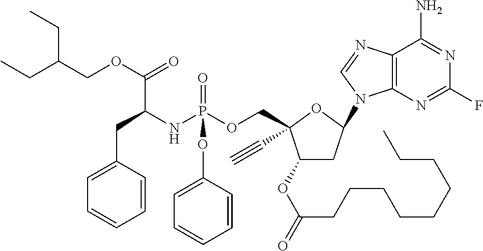 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran- 3-yl decanoate |
| 85 | 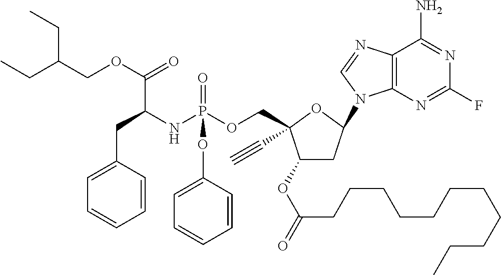 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran- 3-yl dodecanoate |
| 86 | 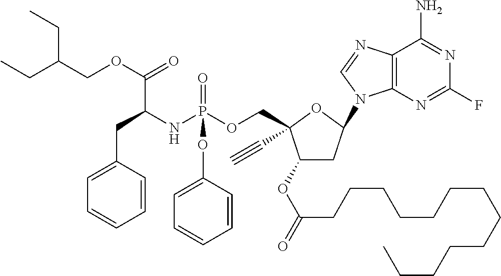 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran- 3-yl tetradecanoate |
| 87 | 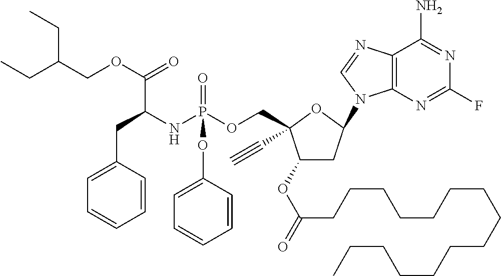 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran- 3-yl palmitate |
| 88 | 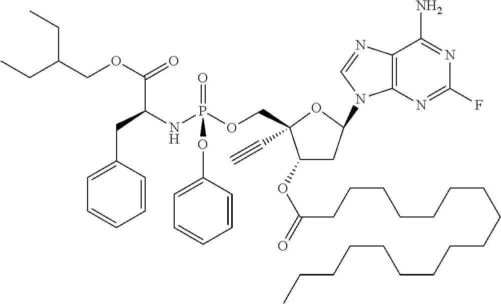 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)CCCCCCCCCCCCCCCCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl) oxy)methyl)-2- ethynyltetrahydrofuran- 3-yl stearate |
| 89 | 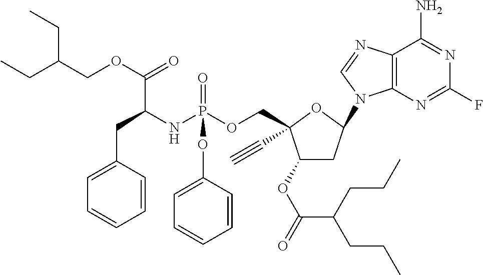 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-((((S)-(((S)-1-(2- ethylbutoxy)-1-oxo-3- phenylpropan-2- yl)amino)(phenoxy)phosphoryl)oxy) methyl)-2-ethynyltetrahydrofuran- 3-yl 2-propylpentanoate |
| 90 | 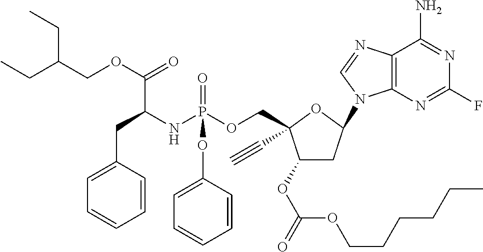 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCC] | 2-Ethylbutyl ((S)-(((2R,3S,5R)-5- (6-amino-2-fluoro-9H-purin-9-yl)- 2-ethynyl-3- (((hexyloxy)carbonyl)oxy) tetrahydrofuran-2- yl)methoxy)(phenoxy)phosphoryl)- L-phenylalaninate |
| 91 | 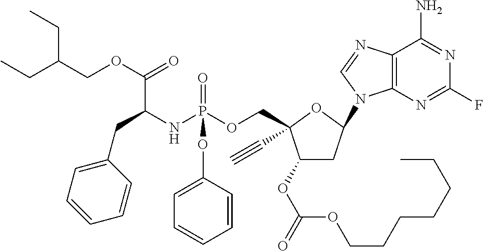 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCC] | 2-Ethylbutyl ((S)-(((2R,3S,5R)-5- (6-amino-2-fluoro-9H-purin-9-yl)- 2-ethynyl-3- (((heptyloxy)carbonyl)oxy) tetrahydrofuran-2- yl)methoxy)(phenoxy)phosphoryl)- L-phenylalaninate |
| 92 | 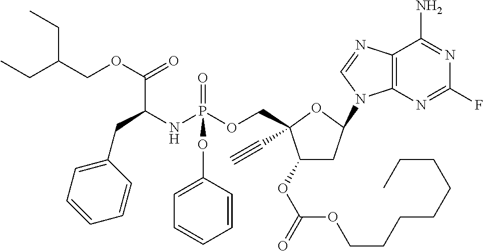 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCCC] | 2-Ethylbutyl ((S)-(((2R,3S,5R)-5- (6-amino-2-fluoro-9H-purin-9-yl)- 2-ethynyl-3- (((octyloxy)carbonyl)oxy) tetrahydrofuran-2- yl)methoxy)(phenoxy)phosphoryl)- L-phenylalaninate |
| 93 | 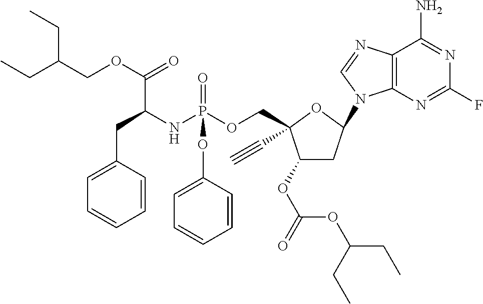 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OC(CC)CC] | 2-Ethylbutyl ((S)-(((2R,3S,5R)-5- (6-amino-2-fluoro-9H-purin-9-yl)- 2-ethynyl-3-(((pentan-3- yloxy)carbonyl)oxy) tetrahydrofuran-2- yl)methoxy)(phenoxy)phosphoryl)- L-phenylalaninate |
| 94 | 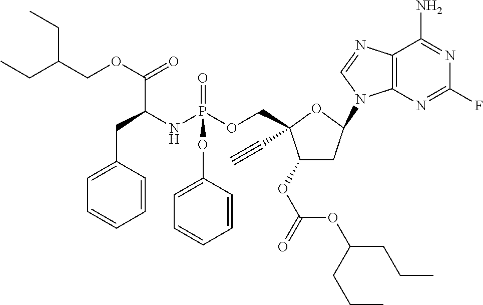 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OC(CCC)CCC] | 2-Ethylbutyl ((S)-(((2R,3S,5R)-5- (6-amino-2-fluoro-9H-purin-9-yl)- 2-ethynyl-3-(((heptan-4- yloxy)carbonyl)oxy) tetrahydrofuran-2- yl)methoxy)(phenoxy)phosphoryl)- L-phenylalaninate |
| 95 | 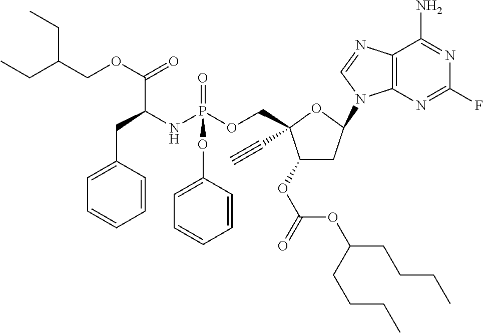 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OC(CCCC)CCCC] | 2-Ethylbutyl ((S)-(((2R,3S,5R)-5- (6-amino-2-fluoro-9H-purin-9-yl)- 2-ethynyl-3-(((nonan-5- yloxy)carbonyl)oxy) tetrahydrofuran-2- yl)methoxy)(phenoxy)phosphoryl)- L-phenylalaninate |
| 96 | 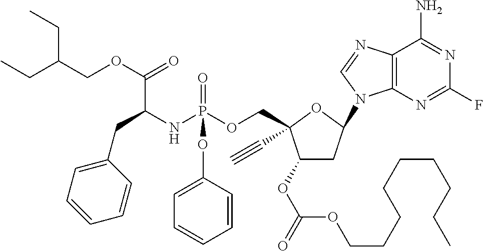 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCCCC] | 2-Ethylbutyl ((S)-(((2R,3S,5R)-5- (6-amino-2-fluoro-9H-purin-9-yl)- 2-ethynyl-3- (((nonyloxy)carbonyl)oxy) tetrahydrofuran-2- yl)methoxy)(phenoxy)phosphoryl)- L-phenylalaninate |
| 97 | 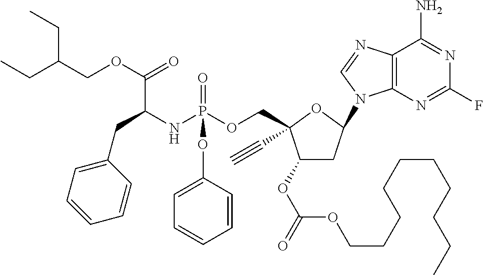 [SMILES: C#C[C@]1(CO[P@@](=O)(N[C@@H](Cc2ccccc2)C(=O)OCC(CC)CC)Oc2ccccc2)O[C@@H](n2cnc3c(N)nc(F)nc32)C[C@@H]1OC(=O)OCCCCCCCCCC] | 2-Ethylbutyl ((S)-(((2R,3S,5R)-5- (6-amino-2-fluoro-9H-purin-9-yl)- 3-(((decyloxy)carbonyl)oxy)-2- ethynyltetrahydrofuran-2- yl)methoxy)(phenoxy)phosphoryl)- L-phenylalaninate |
| 98 | 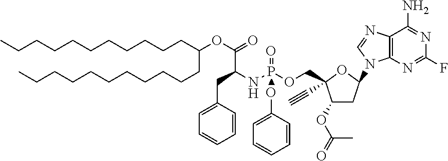 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(C)=O] | Tricosan-12-yl ((S)-(((2R,3S,5R)-3- acetoxy-5-(6-amino-2-fluoro-9H- purin-9-yl)-2- ethynyltetrahydrofuran-2- yl)methoxy)(phenoxy)phosphoryl)- L-phenylalaninate |
| 99 | 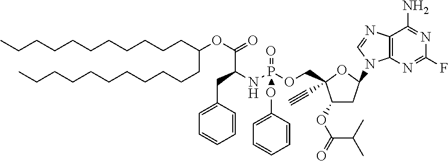 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)C(C)C] | Tricosan-12-yl ((S)-(((2R,3S,5R)-5- (6-amino-2-fluoro-9H-purin-9-yl)- 2-ethynyl-3- (isobutyryloxy)tetrahydrofuran-2- yl)methoxy)(phenoxy)phosphoryl)- L-phenylalaninate |
| 100 | 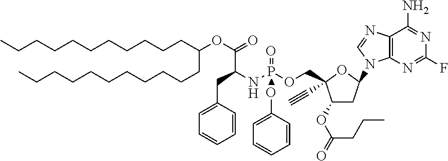 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)CCC] | (2R,3S,5R)-5-(6-Amino-2-fluoro- 9H-purin-9-yl)-2-ethynyl-2-((((S)- (((S)-1-oxo-3-phenyl-1-(tricosan- 12-yloxy)propan-2- yl)amino)(phenoxy)phosphoryl)oxy) methyl)tetrahydrofuran-3-yl butyrate |
| 101 | 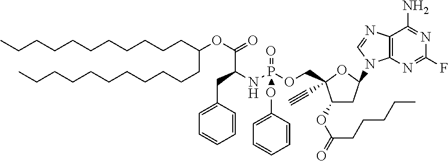 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)CCCCC] | (2R,3S,5R)-5-(6-Amino-2-fluoro- 9H-purin-9-yl)-2-ethynyl-2-((((S)- (((S)-1-oxo-3-phenyl-1-(tricosan- 12-yloxy)propan-2- yl)amino)(phenoxy)phosphoryl)oxy) methyl)tetrahydrofuran-3-yl hexanoate |
| 102 | 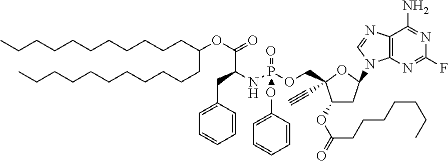 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)CCCCCCC] | (2R,3S,5R)-5-(6-Amino-2-fluoro- 9H-purin-9-yl)-2-ethynyl-2-((((S)- (((S)-1-oxo-3-phenyl-1-(tricosan- 12-yloxy)propan-2- yl)amino)(phenoxy)phosphoryl)oxy) methyl)tetrahydrofuran-3-yl octanoate |
| 103 | 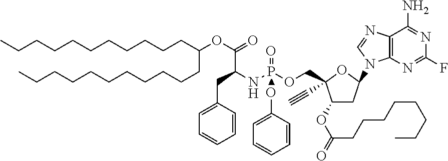 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-ethynyl-2-((((S)- (((S)-1-oxo-3-phenyl-1-(tricosan- 12-yloxy)propan-2- yl)amino)(phenoxy)phosphoryl)oxy) methyl)tetrahydrofuran-3-yl nonanoate |
| 104 | 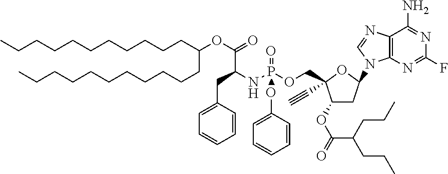 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-ethynyl-2-((((S)- (((S)-1-oxo-3-phenyl-1-(tricosan- 12-yloxy)propan-2- yl)amino)(phenoxy)phosphoryl)oxy) methyl)tetrahydrofuran-3-yl 2- propylpentanoate |
| 105 | 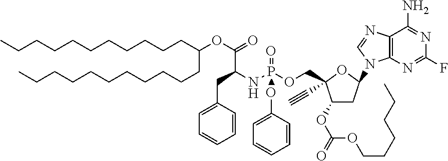 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)OCCCCCC] | Tricosan-12-yl ((S)-(((2R,3S,5R)-5- (6-amino-2-fluoro-9H-purin-9-yl)- 2-ethynyl-3- (((hexyloxy)carbonyl)oxy) tetrahydrofuran-2- yl)methoxy)(phenoxy)phosphoryl)- L-phenylalaninate |
| 106 | 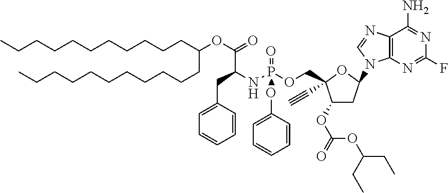 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)OC(CC)CC] | Tricosan-12-yl ((S)-(((2R,3S,5R)-5- (6-amino-2-fluoro-9H-purin-9-yl)- 2-ethynyl-3-(((pentan-3- yloxy)carbonyl)oxy) tetrahydrofuran-2- yl)methoxy)(phenoxy)phosphoryl)- L-phenylalaninate |
| 107 | 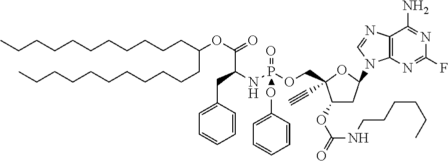 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1OC(=O)NCCCCCC] | Tricosan-12-yl ((S)-(((2R,3S,5R)-5- (6-amino-2-fluoro-9H-purin-9-yl)- 2-ethynyl-3- ((hexylcarbamoyl)oxy) tetrahydrofuran-2- yl)methoxy)(phenoxy)phosphoryl)- L-phenylalaninate |
| 108 | 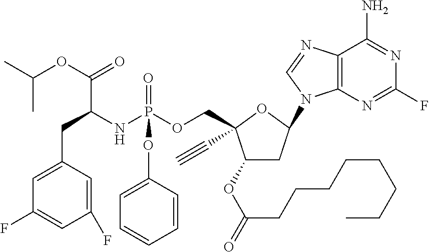 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-((((S)-(((S)-3- (3,5-difluorophenyl)-1-isopropoxy- 1-oxopropan-2- yl)amino)(phenoxy)phosphoryl)oxy) methyl)-2-ethynyltetrahydrofuran- 3-yl nonanoate |
| 109 | 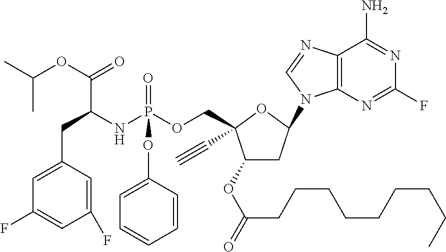 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((S)-(((S)-3- (3,5-difluorophenyl)-1-isopropoxy- 1-oxopropan-2- yl)amino)(phenoxy)phosphoryl)oxy) methyl)-2-ethynyltetrahydrofuran- 3-yl decanoate |
| 110 | 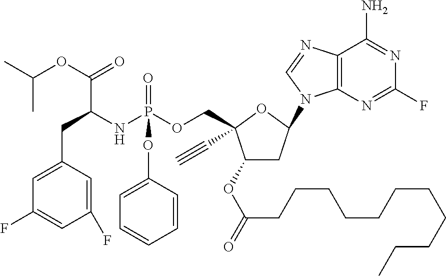 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-((((S)-(((S)-3- (3,5-difluorophenyl)-1-isopropoxy- 1-oxopropan-2- yl)amino)(phenoxy)phosphoryl)oxy )methyl)-2-ethynyltetrahydrofuran- 3-yl dodecanoate |
| 111 | 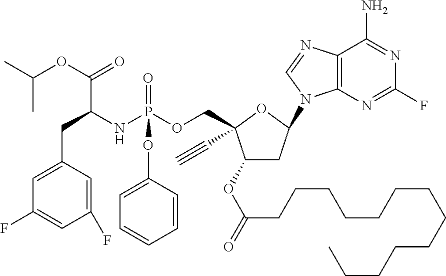 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-((((S)-(((S)-3- (3,5-difluorophenyl)-1-isopropoxy- 1-oxopropan-2- yl)amino)(phenoxy)phosphoryl)oxy) methyl)-2-ethynyltetrahydrofuran- 3-yl tetradecanoate |
| 112 | 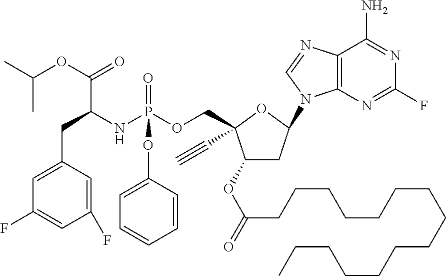 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-((((S)-(((S)-3- (3,5-difluorophenyl)-1-isopropoxy- 1-oxopropan-2- yl)amino)(phenoxy)phosphoryl)oxy) methyl)-2-ethynyltetrahydrofuran- 3-yl palmitate |
| 113 | 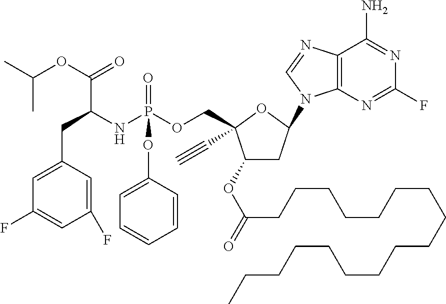 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)CCCCCCCCCCCCCCCCC] | (2R,3S,5R)-5-(6-amino-2-fluoro- 9H-purin-9-yl)-2-((((S)-(((S)-3- (3,5-difluorophenyl)-1-isopropoxy- 1-oxopropan-2- yl)amino)(phenoxy)phosphoryl)oxy) methyl)-2-ethynyltetrahydrofuran- 3-yl stearate |
| 114 | 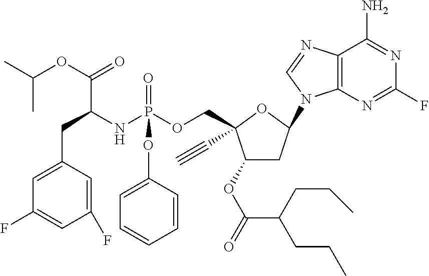 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-((((S)-(((S)-3- (3,5-difluorophenyl)-1-isopropoxy- 1-oxopropan-2- yl)amino)(phenoxy)phosphoryl)oxy) methyl)-2-ethynyltetrahydrofuran- 3-yl 2-propylpentanoate |
| 115 | 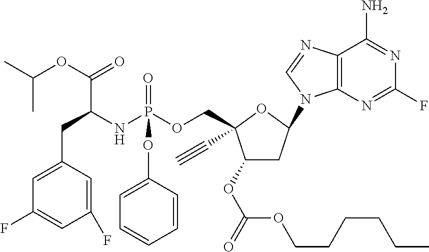 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OCCCCCC] | Isopropyl (S)-2-(((S)-(((2R,3S,5R)- 5-(6-amino-2-fluoro-9H-purin-9- yl)-2-ethynyl-3- (((hexyloxy)carbonyl)oxy) tetrahydrofuran-2- yl)methoxy)(phenoxy)phosphoryl) amino)-3-(3,5- difluorophenyl)propanoate |
| 116 | 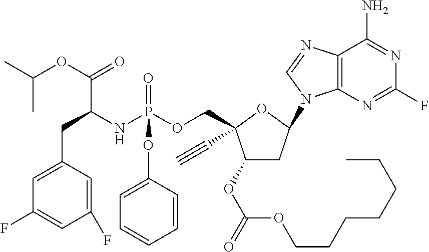 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OCCCCCCC] | Isopropyl (S)-2-(((S)-(((2R,3S,5R)- 5-(6-amino-2-fluoro-9H-purin-9- yl)-2-ethynyl-3- (((heptyloxy)carbonyl)oxy) tetrahydrofuran-2- yl)methoxy)(phenoxy)phosphoryl) amino)-3-(3,5- difluorophenyl)propanoate |
| 117 | 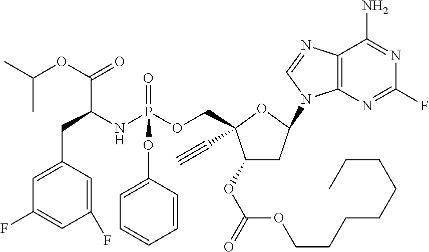 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OCCCCCCCC] | Isopropyl (S)-2-(((S)-(((2R,3S,5R)- 5-(6-amino-2-fluoro-9H-purin-9- yl)-2-ethynyl-3- (((octyloxy)carbonyl)oxy) tetrahydrofuran-2- yl)methoxy)(phenoxy)phosphoryl) amino)-3-(3,5- difluorophenyl)propanoate |
| 118 | 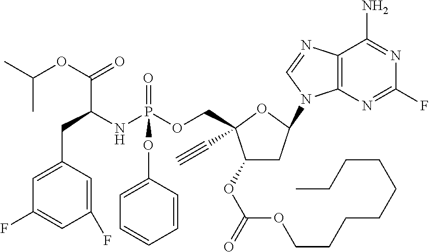 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OCCCCCCCCC] | Isopropyl (S)-2-(((S)-(((2R,3S,5R)- 5-(6-amino-2-fluoro-9H-purin-9- yl)-2-ethynyl-3- (((nonyloxy)carbonyl)oxy) tetrahydrofuran-2- yl)methoxy)(phenoxy)phosphoryl) amino)-3-(3,5- difluorophenyl)propanoate |
| 119 | 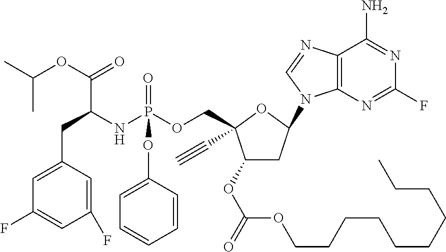 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OCCCCCCCCCC] | Isopropyl (S)-2-(((S)-(((2R,3S,5R)- 5-(6-amino-2-fluoro-9H-purin-9- yl)-3-(((decyloxy)carbonyl)oxy)-2- ethynyltetrahydrofuran-2- yl)methoxy)(phenoxy)phosphoryl) amino)-3-(3,5- difluorophenyl)propanoate |
| 120 | 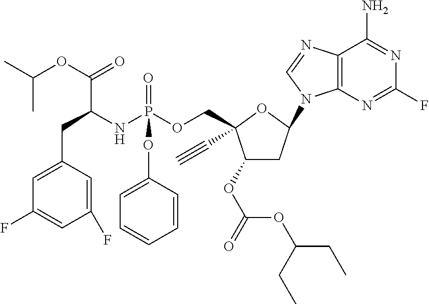 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OC(CC)CC] | Isopropyl (S)-2-(((S)-(((2R,3S,5R)- 5-(6-amino-2-fluoro-9H-purin-9- yl)-2-ethynyl-3-(((pentan-3- yloxy)carbonyl)oxy) tetrahydrofuran-2- yl)methoxy)(phenoxy)phosphoryl) amino)-3-(3,5- difluorophenyl)propanoate |
| 121 | 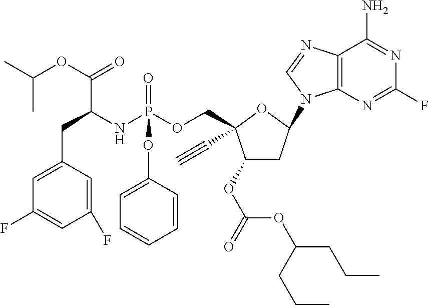 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OC(CCC)CCC] | Isopropyl (S)-2-(((S)-(((2R,3S,5R)- 5-(6-amino-2-fluoro-9H-purin-9- yl)-2-ethynyl-3-(((heptan-4- yloxy)carbonyl)oxy) tetrahydrofuran-2- yl)methoxy)(phenoxy)phosphoryl) amino)-3-(3,5- difluorophenyl)propanoate |
| 122 | 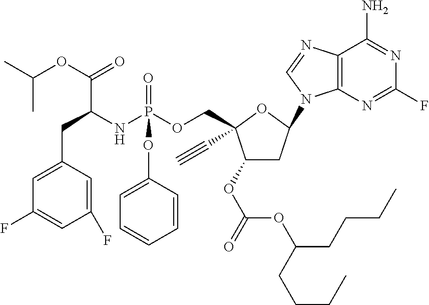 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)OC(CCCC)CCCC] | Isopropyl (S)-2-(((S)-(((2R,3S,5R)- 5-(6-amino-2-fluoro-9H-purin-9- yl)-2-ethynyl-3-(((nonan-5- yloxy)carbonyl)oxy) tetrahydrofuran-2- yl)methoxy)(phenoxy)phosphoryl) amino)-3-(3,5- difluorophenyl)propanoate |
| 123 | 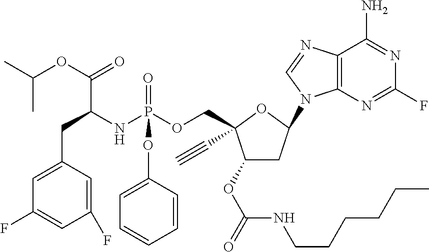 [SMILES: C#C[C@]1(CO[P@@](=O)(N[C@@H](Cc2cc(F)cc(F)c2)C(=O)OC(C)C)Oc2ccccc2)O[C@@H](n2cnc3c(N)nc(F)nc32)C[C@@H]1OC(=O)NCCCCCC] | Isopropyl (S)-2-(((S)-(((2R,3S,5R)- 5-(6-amino-2-fluoro-9H-purin-9- yl)-2-ethynyl-3- ((hexylcarbamoyl)oxy) tetrahydrofuran- 2-yl)methoxy)(phenoxy)phosphoryl) amino)-3-(3,5- difluorophenyl)propanoate |
| 124 | 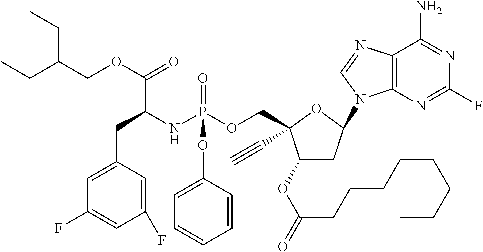 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCC] | (2R,3S,5R)-5-(6-Amino-2-fluoro- 9H-purin-9-yl)-2-((((S)-(((S)-3- (3,5-difluorophenyl)-1-(2- ethylbutoxy)-1-oxopropan-2- yl)amino)(phenoxy)phosphoryl)oxy) methyl)-2-ethynyltetrahydrofuran- 3-yl nonanoate |
| 125 | 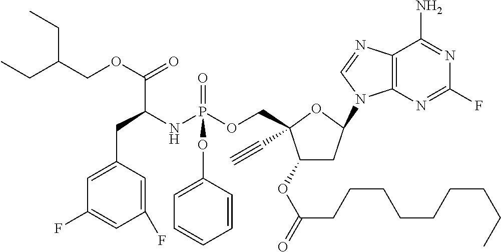 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCC] | (2R,3S,5R)-5-(6-Amino-2-fluoro- 9H-purin-9-yl)-2-((((S)-(((S)-3- (3,5-difluorophenyl)-1-(2- ethylbutoxy)-1-oxopropan-2- yl)amino)(phenoxy)phosphoryl)oxy) methyl)-2-ethynyltetrahydrofuran- 3-yl decanoate |
| 126 | 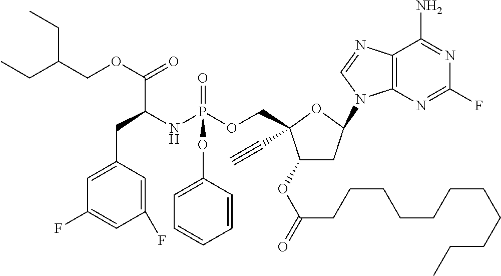 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCC] | (2R,3S,5R)-5-(6-Amino-2-fluoro- 9H-purin-9-yl)-2-((((S)-(((S)-3- (3,5-difluorophenyl)-1-(2- ethylbutoxy)-1-oxopropan-2- yl)amino)(phenoxy)phosphoryl)oxy) methyl)-2-ethynyltetrahydrofuran- 3-yl dodecanoate |
| 127 | 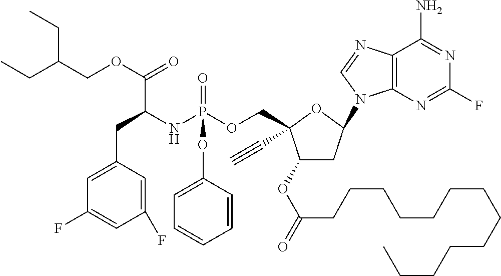 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCCCC] | (2R,3S,5R)-5-(6-Amino-2-fluoro- 9H-purin-9-yl)-2-((((S)-(((S)-3- (3,5-difluorophenyl)-1-(2- ethylbutoxy)-1-oxopropan-2- yl)amino)(phenoxy)phosphoryl)oxy) methyl)-2-ethynyltetrahydrofuran- 3-yl tetradecanoate |
| 128 | 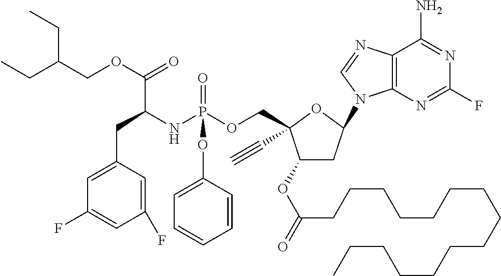 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCCCCCC] | (2R,3S,5R)-5-(6-Amino-2-fluoro- 9H-purin-9-yl)-2-((((S)-(((S)-3- (3,5-difluorophenyl)-1-(2- ethylbutoxy)-1-oxopropan-2- yl)amino)(phenoxy)phosphoryl)oxy) methyl)-2-ethynyltetrahydrofuran- 3-yl palmitate |
| 129 | 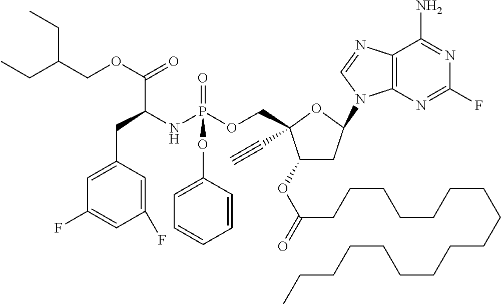 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)CCCCCCCCCCCCCCCCC] | (2R,3S,5R)-5-(6-Amino-2-fluoro- 9H-purin-9-yl)-2-((((S)-(((S)-3- (3,5-difluorophenyl)-1-(2- ethylbutoxy)-1-oxopropan-2- yl)amino)(phenoxy)phosphoryl)oxy) methyl)-2-ethynyltetrahydrofuran- 3-yl stearate |
| 130 | 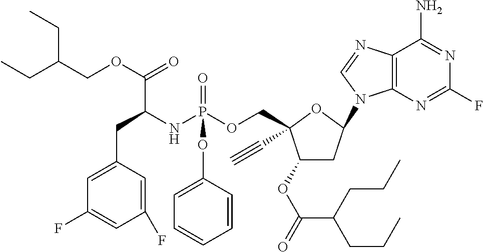 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)C(CCC)CCC] | (2R,3S,5R)-5-(6-Amino-2-fluoro- 9H-purin-9-yl)-2-((((S)-(((S)-3- (3,5-difluorophenyl)-1-(2- ethylbutoxy)-1-oxopropan-2- yl)amino)(phenoxy)phosphoryl)oxy) methyl)-2-ethynyltetrahydrofuran- 3-yl 2-propylpentanoate |
| 131 | 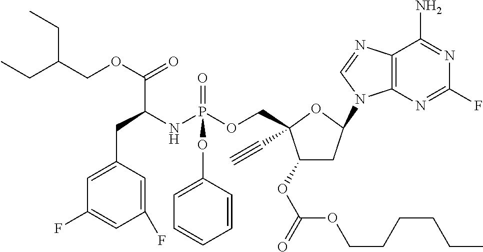 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OCCCCCC] | 2-Ethylbutyl (S)-2-(((S)- (((2R,3S,5R)-5-(6-amino-2-fluoro- 9H-purin-9-yl)-2-ethynyl-3- (((hexyloxy)carbonyl)oxy) tetrahydrofuran-2- yl)methoxy)(phenoxy)phosphoryl) amino)-3-(3,5- difluorophenyl)propanoate |
| 132 | 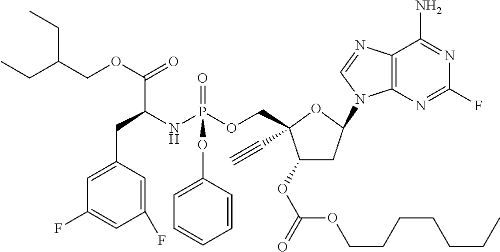 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OCCCCCCC] | 2-Ethylbutyl (S)-2-(((S)- (((2R,3S,5R)-5-(6-amino-2-fluoro- 9H-purin-9-yl)-2-ethynyl-3- (((heptyloxy)carbonyl)oxy) tetrahydrofuran-2- yl)methoxy)(phenoxy)phosphoryl) amino)-3-(3,5- difluorophenyl)propanoate |
| 133 | 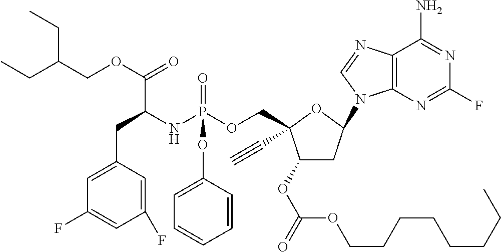 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OCCCCCCCC] | 2-Ethylbutyl (S)-2-(((S)- (((2R,3S,5R)-5-(6-amino-2-fluoro- 9H-purin-9-yl)-2-ethynyl-3- (((octyloxy)carbonyl)oxy) tetrahydrofuran-2- yl)methoxy)(phenoxy)phosphoryl) amino)-3-(3,5- difluorophenyl)propanoate |
| 134 | 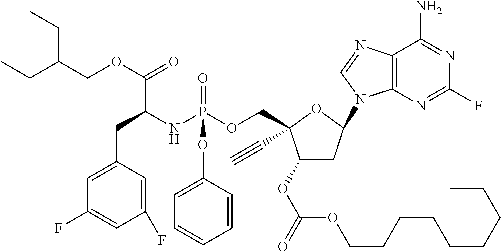 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OCCCCCCCCC] | 2-Ethylbutyl (S)-2-(((S)- (((2R,3S,5R)-5-(6-amino-2-fluoro- 9H-purin-9-yl)-2-ethynyl-3- (((nonyloxy)carbonyl)oxy) tetrahydrofuran-2- yl)methoxy)(phenoxy)phosphoryl) amino)-3-(3,5- difluorophenyl)propanoate |
| 135 | 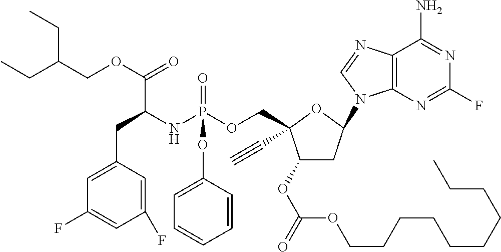 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OCCCCCCCCCC] | 2-Ethylbutyl (S)-2-(((S)- (((2R,3S,5R)-5-(6-amino-2-fluoro- 9H-purin-9-yl)-3- (((decyloxy)carbonyl)oxy)-2- ethynyltetrahydrofuran-2- yl)methoxy)(phenoxy) phosphoryl)amino)- 3-(3,5-difluorophenyl)propanoate |
| 136 | 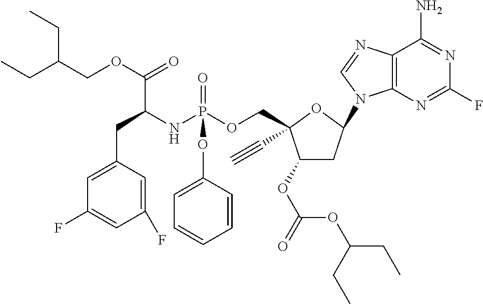 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OC(CC)CC] | 2-Ethylbutyl (S)-2-(((S)- (((2R,3S,5R)-5-(6-amino-2-fluoro- 9H-purin-9-yl)-2- ethynyl-3-(((pentan-3- yloxy)carbonyl)oxy) tetrahydrofuran-2- yl)methoxy)(phenoxy)phosphoryl) amino)-3-(3,5- difluorophenyl)propanoate |
| 137 | 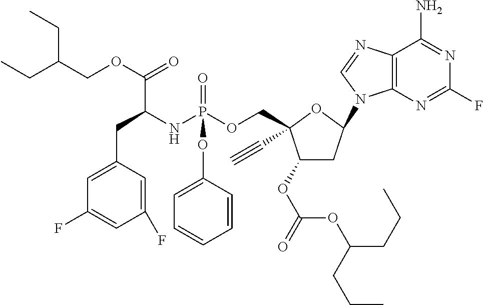 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OC(CCC)CCC] | 2-Ethylbutyl (S)-2-(((S)- (((2R,3S,5R)-5-(6-amino-2-fluoro- 9H-purin-9-yl)-2-ethynyl- 3-(((heptan-4- yloxy)carbonyl)oxy) tetrahydrofuran-2- yl)methoxy)(phenoxy)phosphoryl) amino)-3-(3,5- difluorophenyl)propanoate |
| 138 | 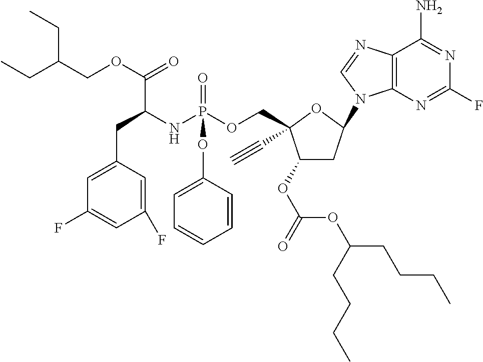 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)OC(CCCC)CCCC] | 2-Ethylbutyl (S)-2-(((S)- (((2R,3S,5R)-5-(6-amino-2-fluoro- 9H-purin-9-yl)-2- ethynyl-3-(((nonan-5- yloxy)carbonyl)oxy) tetrahydrofuran-2- yl)methoxy)(phenoxy)phosphoryl) amino)-3-(3,5- difluorophenyl)propanoate |
| 139 | 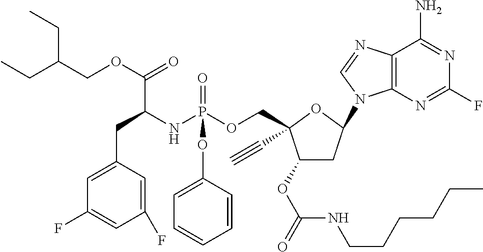 [SMILES: C#C[C@]1(CO[P@@](=O)(N[C@@H](Cc2cc(F)cc(F)c2)C(=O)OCC(CC)CC)Oc2ccccc2)O[C@@H](n2cnc3c(N)nc(F)nc32)C[C@@H]1OC(=O)NCCCCCC] | 2-Ethylbutyl (S)-2-(((S)- (((2R,3S,5R)-5-(6-amino-2-fluoro- 9H-purin-9-yl)-2-ethynyl-3- ((hexylcarbamoyl)oxy) tetrahydrofuran-2- yl)methoxy)(phenoxy)phosphoryl) amino)-3-(3,5- difluorophenyl)propanoate |

and pharmaceutically acceptable salts thereof.

27. A pharmaceutical composition comprising a compound of claim 1, or a pharmaceutically acceptable salt thereof, and a pharmaceutically acceptable excipient.

28. The composition of claim 27, wherein the composition is present in parenteral form.

29. The composition of claim 27, wherein the composition is in a tablet form.

30. The composition of claim 27, wherein the composition is formulated as a long acting parenteral injection.

31. A combination comprising a compound of claim 1 or a pharmaceutically acceptable salt thereof and one or more pharmaceutical agents active against HIV.

32. A method of treating an HIV infection in a patient comprising administering to the subject a compound of claim 1, or a pharmaceutically acceptable salt thereof.

33. A method of preventing an HIV infection in a patient at risk for developing an HIV infection, comprising administering to the subject a compound of claim 1, or a pharmaceutically acceptable salt thereof.

34. A method of treating an HIV infection in a patient comprising administering to the subject a combination of claim 31.

35. A method of preventing an HIV infection in a patient at risk for developing an HIV infection, comprising administering to the subject to the subject a combination of claim 31.

36. (canceled)

37. (canceled)

38. (canceled)

39. (canceled)

40. (canceled)

41. (canceled)
